SEQUENCE LISTING (610 sequences)

SEQ ID NO: 1 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 241 aa, Macaca fascicularis
MGWSCIILFLVATATGVHSMFTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERT

SEQ ID NO: 3 - protein, 245 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHHHHHH

SEQ ID NO: 4 - protein, 475 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTIEGREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 249 aa, Macaca fascicularis
MGWSCIILFLVATATGVHSMFTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERTDYKDDDDK

SEQ ID NO: 6 - protein, 405 aa, Homo sapiens
MGWSCIILFLVATATGVHSLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQKLENLYFQGIEGRMDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 7 - protein, 8 aa, Homo sapiens
GFTFDDYA

SEQ ID NO: 8 - protein, 8 aa, Homo sapiens
ISWKSNII

SEQ ID NO: 9 - protein, 15 aa, Homo sapiens
ARDITGSGSYGWFDP

SEQ ID NO: 10 - protein, 5 aa, Homo sapiens
DYAMH

SEQ ID NO: 11 - protein, 17 aa, Homo sapiens
GISWKSNIIGYADSVKG

SEQ ID NO: 12 - protein, 13 aa, Homo sapiens
DITGSGSYGWFDP

SEQ ID NO: 13 - protein, 122 aa, Homo sapiens
EVQLVESGGGLVQPGRSLRLSCAASGFTFDDYAMHWVRQTPGKGLEWVSGISWKSNIIGYADSVKGRFTISRDNAKNSLYLQMNSLRAEDTALYYCARDITGSGSYGWFDPWGQGTLVTVSS

SEQ ID NO: 14 - DNA, 508 bp, Homo sapiens
  source 1..508: mol_type = unassigned DNA
caagaaaaagcttgccgccaccatggagtttgggctgagctggattttccttttggctattttaaaaggtgtccagtgtgaagtacaattggtggagtccgggggaggcttggtacagcctggcaggtccctgagactctcctgtgcagcctctggattcacctttgatgattatgccatgcactgggtccgacaaactccagggaagggcctggagtgggtctcaggtataagttggaagagtaatatcataggctatgcggactctgtgaagggccgattcaccatctccagagacaacgccaagaactccctgtatctgcaaatgaacagtctgagagctgaggacacggccttgtattattgtgcaagagatataacgggttcggggagttatggctggttcgacccctggggccagggaaccctggtcaccgtctcctcagccaaaacgacacccccatctgtctatccactggcccctgaatctgctaaaactcagcctccg

SEQ ID NO: 15 - protein, 449 aa, Homo sapiens
EVQLVESGGGLVQPGRSLRLSCAASGFTFDDYAMHWVRQTPGKGLEWVSGISWKSNIIGYADSVKGRFTISRDNAKNSLYLQMNSLRAEDTALYYCARDITGSGSYGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - DNA, 1356 bp, Homo sapiens
  source 1..1356: mol_type = unassigned DNA
gaagtgcagctggtggaatctggcggcggactggtgcagcctggcagatccctgagactgtcttgtgccgcctccggcttcaccttcgacgactacgctatgcactgggtgcgacagacccctggcaagggcctggaatgggtgtccggcatctcctggaagtccaacatcatcggctacgccgactccgtgaagggccggttcaccatctcccgggacaacgccaagaactccctgtacctgcagatgaacagcctgcgggccgaggacaccgccctgtactactgcgccagagacatcaccggctccggctcctacggatggttcgatccttggggccagggcaccctcgtgaccgtgtcctctgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 17 - protein, 6 aa, Homo sapiens
QSISSY

SEQ ID NO: 18 - skipped

SEQ ID NO: 19 - protein, 9 aa, Homo sapiens
QQSYSNPIT

SEQ ID NO: 20 - protein, 11 aa, Homo sapiens
RASQSISSYLN

SEQ ID NO: 21 - protein, 7 aa, Homo sapiens
VASSLQS

SEQ ID NO: 22 - protein, 9 aa, Homo sapiens
QQSYSNPIT

SEQ ID NO: 23 - protein, 107 aa, Homo sapiens
DIQMTQSPSSLSASVGDRVTITCRASQSISSYLNWYQQKPGKAPKPLIYVASSLQSGVPSSFSGSGSGTDFTLTISSLQPEDFATYYCQQSYSNPITFGQGTRLEIK

SEQ ID NO: 24 - DNA, 321 bp, Homo sapiens
  source 1..321: mol_type = unassigned DNA
gacatccagatgacccagtctccatcctccctgtctgcatctgtaggagacagagtcaccatcacttgccgggcaagtcagagcattagcagctatttaaattggtatcagcagaaaccagggaaagcccctaagcccctgatctatgttgcatccagtttgcaaagtggggtcccatcaagtttcagtggcagtggatctgggacagatttcactctcaccatcagcagtctgcaacctgaagattttgcaacttactactgtcaacagagttacagtaatccgatcaccttcggccaagggacacgactggagatcaaa

SEQ ID NO: 25 - protein, 214 aa, Homo sapiens
DIQMTQSPSSLSASVGDRVTITCRASQSISSYLNWYQQKPGKAPKPLIYVASSLQSGVPSSFSGSGSGTDFTLTISSLQPEDFATYYCQQSYSNPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 642 bp, Homo sapiens
  source 1..642: mol_type = unassigned DNA
gacatccagatgacccagtctccatcctccctgtctgcatctgtaggagacagagtcaccatcacttgccgggcaagtcagagcattagcagctatttaaattggtatcagcagaaaccagggaaagcccctaagcccctgatctatgttgcatccagtttgcaaagtggggtcccatcaagtttcagtggcagtggatctgggacagatttcactctcaccatcagcagtctgcaacctgaagattttgcaacttactactgtcaacagagttacagtaatccgatcaccttcggccaagggacacgactggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 27 - protein, 8 aa, Homo sapiens
GFTFDDYA

SEQ ID NO: 28 - protein, 8 aa, Homo sapiens
ISWIRTGI

SEQ ID NO: 29 - protein, 16 aa, Homo sapiens
AKDMKGSGTYGGWFDT

SEQ ID NO: 30 - protein, 5 aa, Homo sapiens
DYAMH

SEQ ID NO: 31 - protein, 17 aa, Homo sapiens
GISWIRTGIGYADSVKG

SEQ ID NO: 32 - protein, 14 aa, Homo sapiens
DMKGSGTYGGWFDT

SEQ ID NO: 33 - protein, 123 aa, Homo sapiens
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSS

SEQ ID NO: 34 - DNA, 482 bp, Homo sapiens
  source 1..482: mol_type = unassigned DNA
aagcttgccgccaccatggagtttgggctgagctggattttccttttggctattttaaaaggtgtccagtgtgaagtgcagctggtggagtctgggggaggcttggtgcagcctggcaggtccctgagactctcctgtgcagcctctggattcacctttgatgattatgccatgcactgggtccggcaagttccagggaagggcctggaatgggtctcaggcattagttggattcgtactggcataggctatgcggactctgtgaagggccgattcaccattttcagagacaacgccaagaattccctgtatctgcaaatgaacagtctgagagctgaggacacggccttgtattactgtgcaaaagatatgaagggttcggggacttatggggggtggttcgacacctggggccagggaaccctggtcaccgtctcctcagccaaaacaacagccccatcggtctatccactggcccctgc

SEQ ID NO: 35 - protein, 450 aa, Homo sapiens
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - DNA, 1359 bp, Homo sapiens
  source 1..1359: mol_type = unassigned DNA
gaagtgcagctggtggaatctggcggcggactggtgcagcctggcagatccctgagactgtcttgtgccgcctccggcttcaccttcgacgactacgctatgcactgggtgcgacaggtgccaggcaagggcctggaatgggtgtccggcatctcttggatccggaccggcatcggctacgccgactctgtgaagggccggttcaccatcttccgggacaacgccaagaactccctgtacctgcagatgaacagcctgcgggccgaggacaccgccctgtactactgcgccaaggacatgaagggctccggcacctacggcggatggttcgatacttggggccagggcaccctcgtgaccgtgtcctctgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 37 - protein, 6 aa, Homo sapiens
QSISSY

SEQ ID NO: 38 - skipped

SEQ ID NO: 39 - protein, 9 aa, Homo sapiens
QQSYSTPIT

SEQ ID NO: 40 - protein, 11 aa, Homo sapiens
RASQSISSYLN

SEQ ID NO: 41 - protein, 7 aa, Homo sapiens
VASSLQS

SEQ ID NO: 42 - protein, 9 aa, Homo sapiens
QQSYSTPIT

SEQ ID NO: 43 - protein, 107 aa, Homo sapiens
DIQMTQSPSSLSASVGDRVTITCRASQSISSYLNWYQQKPGKAPKLLIYVASSLQSGVPSRFSGSGSGTDFTLTISSLQPEDFATYYCQQSYSTPITFGQGTRLEIK

SEQ ID NO: 44 - DNA, 418 bp, Homo sapiens
  source 1..418: mol_type = unassigned DNA
aaagcttgccgccaccatgaggctccctgctcagcttctggggctcctgctactctggctccgaggtgccagatgtgacatccagatgacccagtctccatcctccctgtctgcatctgtaggagacagagtcaccatcacttgccgggcaagtcagagcattagcagctatttaaattggtatcagcagaaaccagggaaagcccctaaactcctgatctatgttgcatccagtttgcaaagtggggtcccatcaaggttcagtggcagtggatctgggacagatttcactctcactatcagcagtctgcaacctgaagattttgcaacttactactgtcaacagagttacagtaccccgatcaccttcggccaagggacacgtctggagatcaaacgtacggatgctgcaccaact

SEQ ID NO: 45 - protein, 214 aa, Homo sapiens
DIQMTQSPSSLSASVGDRVTITCRASQSISSYLNWYQQKPGKAPKLLIYVASSLQSGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - DNA, 642 bp, Homo sapiens
  source 1..642: mol_type = unassigned DNA
gacatccagatgacccagtccccctccagcctgtctgcttccgtgggcgacagagtgaccatcacctgtcgggcctcccagtccatctcctcctacctgaactggtatcagcagaagcccggcaaggcccccaagctgctgatctacgtggccagctctctgcagtccggcgtgccctctagattctccggctctggctctggcaccgactttaccctgaccatcagctccctgcagcccgaggacttcgccacctactactgccagcagtcctactccacccctatcaccttcggccagggcacccggctggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 47 - protein, 450 aa, Homo sapiens
EVQLVESGGGLVQPGRSLRLSCAASGFTFDDYAMHWVRQAPGKGLEWVSGISWIRTGIGYADSVKGRFTIFRDNAKNSLYLQMNSLRAEDTALYYCAKDMKGSGTYGGWFDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 450 aa, Homo sapiens
EVQLVESGGGLVQPGRSLRLSCAASGFTFDDYAMHWVRQVPGKGLEWVSGISWIRTGIGYADSVKGRFTISRDNAKNSLYLQMNSLRAEDTALYYCAKDMKGSGTYGGWFDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 49 - protein, 448 aa, Homo sapiens
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 214 aa, Homo sapiens
DIQMTQSPSSLSASVGDRVTITCRASQSISSYLNWYQQKPGKAPKLFIYVASSLQSGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 8 aa, Homo sapiens
GFTFSSYW

SEQ ID NO: 53 - protein, 8 aa, Homo sapiens
IKEDGSEK

SEQ ID NO: 54 - protein, 12 aa, Homo sapiens
ARNRLYSDFLDN

SEQ ID NO: 55 - protein, 5 aa, Homo sapiens
SYWMS

SEQ ID NO: 56 - protein, 17 aa, Homo sapiens
NIKEDGSEKYYVDSVKG

SEQ ID NO: 57 - protein, 10 aa, Homo sapiens
NRLYSDFLDN

SEQ ID NO: 58 - protein, 119 aa, Homo sapiens
EVQLVESGGGLVQPGGSLRLSCAASGFTFSSYWMSWVRQAPGKGLEWVANIKEDGSEKYYVDSVKGRFTISRDNAKNSLYLQMNSLRAEDTSVYYCARNRLYSDFLDNWGQGTLVTVSS

SEQ ID NO: 59 - DNA, 358 bp, Homo sapiens
  source 1..358: mol_type = unassigned DNA
gaggtgcagctggtggagtctgggggaggcttggtccagcctggggggtccctgagactctcctgtgcagcctctggattcacgtttagtagctattggatgagttgggtccgccaggctccagggaaggggctggagtgggtggccaacatcaaagaagatggaagtgagaaatactatgtcgactctgtgaagggccgattcaccatctccagagacaacgccaagaactcactgtatctgcaaatgaacagcctgagagccgaggacacgtctgtgtattactgtgcgagaaatcgactctacagtgacttccttgacaactggggccagggaaccctggtcaccgtctcctcag

SEQ ID NO: 60 - protein, 449 aa, Homo sapiens
EVQLVESGGGLVQPGGSLRLSCAASGFTFSSYWMSWVRQAPGKGLEWVANIKEDGSEKYYVDSVKGRFTISRDNAKNSLYLQMNSLRAEDTSVYYCARNRLYSDFLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - DNA, 1347 bp, Homo sapiens
  source 1..1347: mol_type = unassigned DNA
gaggtgcagctggtggagtctgggggaggcttggtccagcctggggggtccctgagactctcctgtgcagcctctggattcacgtttagtagctattggatgagttgggtccgccaggctccagggaaggggctggagtgggtggccaacatcaaagaagatggaagtgagaaatactatgtcgactctgtgaagggccgattcaccatctccagagacaacgccaagaactcactgtatctgcaaatgaacagcctgagagccgaggacacgtctgtgtattactgtgcgagaaatcgactctacagtgacttccttgacaactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 62 - protein, 6 aa, Homo sapiens
QGVSSW

SEQ ID NO: 63 - skipped

SEQ ID NO: 64 - protein, 9 aa, Homo sapiens
QQANSIPFT

SEQ ID NO: 65 - protein, 11 aa, Homo sapiens
RASQGVSSWLA

SEQ ID NO: 66 - protein, 7 aa, Homo sapiens
GASSLQS

SEQ ID NO: 67 - protein, 9 aa, Homo sapiens
QQANSIPFT

SEQ ID NO: 68 - protein, 107 aa, Homo sapiens
DIQMTQSPSSVSASVGDRVTITCRASQGVSSWLAWYQQKSGKAPKLLIYGASSLQSGVPSRFSGSGSGTEFILTISSLQPEDFATYYCQQANSIPFTFGPGTKVDIK

SEQ ID NO: 69 - DNA, 322 bp, Homo sapiens
  source 1..322: mol_type = unassigned DNA
gacatccagatgacccagtctccatcttccgtgtctgcatctgtcggagacagagtcaccatcacttgtcgggcgagtcagggtgttagcagctggttagcctggtatcagcagaaatcagggaaagcccctaagctcctgatctatggtgcatccagtttgcaaagtggggtcccatcaagattcagcggcagtggatctgggacagagttcattctcaccatcagcagcctgcagcctgaagattttgcaacttactattgtcaacaggctaacagtatcccattcactttcggccctgggaccaaagtggatatcaaac

SEQ ID NO: 70 - protein, 214 aa, Homo sapiens
DIQMTQSPSSVSASVGDRVTITCRASQGVSSWLAWYQQKSGKAPKLLIYGASSLQSGVPSRFSGSGSGTEFILTISSLQPEDFATYYCQQANSIPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - DNA, 642 bp, Homo sapiens
  source 1..642: mol_type = unassigned DNA
gacatccagatgacccagtctccatcttccgtgtctgcatctgtcggagacagagtcaccatcacttgtcgggcgagtcagggtgttagcagctggttagcctggtatcagcagaaatcagggaaagcccctaagctcctgatctatggtgcatccagtttgcaaagtggggtcccatcaagattcagcggcagtggatctgggacagagttcattctcaccatcagcagcctgcagcctgaagattttgcaacttactattgtcaacaggctaacagtatcccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 72 - protein, 8 aa, Homo sapiens
GFTFSSYW

SEQ ID NO: 73 - protein, 8 aa, Homo sapiens
IKEDGSEK

SEQ ID NO: 74 - protein, 12 aa, Homo sapiens
ARVRLYSDFLDY

SEQ ID NO: 75 - protein, 5 aa, Homo sapiens
SYWMS

SEQ ID NO: 76 - protein, 17 aa, Homo sapiens
NIKEDGSEKYYVDSLKG

SEQ ID NO: 77 - protein, 10 aa, Homo sapiens
VRLYSDFLDY

SEQ ID NO: 78 - protein, 119 aa, Homo sapiens
EVQLVDSGGGLVQPGGSLRLSCAASGFTFSSYWMSWVRQAPGKGLEWVANIKEDGSEKYYVDSLKGRFTISRDNAKNSLYLQMNSLRAEDTSVYYCARVRLYSDFLDYWGQGTLVTVSS

SEQ ID NO: 79 - DNA, 358 bp, Homo sapiens
  source 1..358: mol_type = unassigned DNA
gaggtgcagctggtggactctgggggaggcttggtccagcctggggggtccctgagactctcctgtgcagcctctggattcacgtttagtagctattggatgagttgggtccgccaggctccaggaaaggggctggagtgggtggccaacataaaagaagatggaagtgagaaatactatgtagactctttgaagggccgattcaccatctccagagacaacgccaagaactcactgtatctgcaaatgaacagcctgagagccgaggacacgtctgtgtattactgtgcgagagttcgactctacagtgacttccttgactactggggccagggaaccctggtcaccgtctcctcag

SEQ ID NO: 80 - protein, 449 aa, Homo sapiens
EVQLVDSGGGLVQPGGSLRLSCAASGFTFSSYWMSWVRQAPGKGLEWVANIKEDGSEKYYVDSLKGRFTISRDNAKNSLYLQMNSLRAEDTSVYYCARVRLYSDFLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - DNA, 1347 bp, Homo sapiens
  source 1..1347: mol_type = unassigned DNA
gaggtgcagctggtggactctgggggaggcttggtccagcctggggggtccctgagactctcctgtgcagcctctggattcacgtttagtagctattggatgagttgggtccgccaggctccaggaaaggggctggagtgggtggccaacataaaagaagatggaagtgagaaatactatgtagactctttgaagggccgattcaccatctccagagacaacgccaagaactcactgtatctgcaaatgaacagcctgagagccgaggacacgtctgtgtattactgtgcgagagttcgactctacagtgacttccttgactactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 82 - protein, 6 aa, Homo sapiens
QGVSSW

SEQ ID NO: 83 - skipped

SEQ ID NO: 84 - protein, 9 aa, Homo sapiens
QQANSIPFT

SEQ ID NO: 85 - protein, 11 aa, Homo sapiens
RASQGVSSWLA

SEQ ID NO: 86 - protein, 7 aa, Homo sapiens
GASSLQS

SEQ ID NO: 87 - protein, 9 aa, Homo sapiens
QQANSIPFT

SEQ ID NO: 88 - protein, 107 aa, Homo sapiens
DIQMTQSPSSVSASVGDRVTITCRASQGVSSWLAWYQQKSGKAPKLLIYGASSLQSGVPSRFSGSGSGTEFILSISSLQPEDFATYYCQQANSIPFTFGPGTKVDIK

SEQ ID NO: 89 - DNA, 322 bp, Homo sapiens
  source 1..322: mol_type = unassigned DNA
gacatccagatgacccagtctccatcttccgtgtctgcatctgtcggagacagagtcaccatcacttgtcgggcgagtcagggtgttagcagttggttagcctggtatcagcagaaatcagggaaagcccctaagctcctgatctatggtgcctccagtttgcaaagtggggtcccatcaagattcagcggcagtggatctgggacagagttcattctcagcatcagcagcctgcagcctgaagattttgcaacttactattgtcaacaggctaacagtatcccattcactttcggccctgggaccaaagtggatatcaaac

SEQ ID NO: 90 - protein, 214 aa, Homo sapiens
DIQMTQSPSSVSASVGDRVTITCRASQGVSSWLAWYQQKSGKAPKLLIYGASSLQSGVPSRFSGSGSGTEFILSISSLQPEDFATYYCQQANSIPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - DNA, 642 bp, Homo sapiens
  source 1..642: mol_type = unassigned DNA
gacatccagatgacccagtctccatcttccgtgtctgcatctgtcggagacagagtcaccatcacttgtcgggcgagtcagggtgttagcagttggttagcctggtatcagcagaaatcagggaaagcccctaagctcctgatctatggtgcctccagtttgcaaagtggggtcccatcaagattcagcggcagtggatctgggacagagttcattctcagcatcagcagcctgcagcctgaagattttgcaacttactattgtcaacaggctaacagtatcccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 92 - protein, 9 aa, Homo sapiens
GGSIISSDW

SEQ ID NO: 93 - protein, 7 aa, Homo sapiens
IFHSGRT

SEQ ID NO: 94 - protein, 8 aa, Homo sapiens
ARDGSGSY

SEQ ID NO: 95 - protein, 6 aa, Homo sapiens
SSDWWN

SEQ ID NO: 96 - protein, 16 aa, Homo sapiens
EIFHSGRTNYNPSLKS

SEQ ID NO: 97 - protein, 6 aa, Homo sapiens
DGSGSY

SEQ ID NO: 98 - protein, 115 aa, Homo sapiens
QVQLQESGPGLVKPSGTLSLTCIVSGGSIISSDWWNWVRQPPGKGLEWIGEIFHSGRTNYNPSLKSRVTISIDKSKNQFSLRLSSVTAADTAVYYCARDGSGSYWGQGTLVTVSS

SEQ ID NO: 99 - DNA, 346 bp, Homo sapiens
  source 1..346: mol_type = unassigned DNA
caggtgcagctgcaggagtcgggcccaggactggtgaagccttcggggaccctgtccctcacctgcattgtctctggtggctccatcatcagtagtgactggtggaattgggtccgccagcccccagggaaggggctggagtggattggagaaatctttcatagtgggaggaccaactacaacccgtccctcaagagtcgagtcaccatatcaatagacaagtccaagaatcagttctccctgaggctgagctctgtgaccgccgcggacacggccgtgtattactgtgcgagagatggttcggggagttactggggccagggaaccctggtcaccgtctcctcag

SEQ ID NO: 100 - protein, 445 aa, Homo sapiens
QVQLQESGPGLVKPSGTLSLTCIVSGGSIISSDWWNWVRQPPGKGLEWIGEIFHSGRTNYNPSLKSRVTISIDKSKNQFSLRLSSVTAADTAVYYCARDGSGSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - DNA, 1335 bp, Homo sapiens
  source 1..1335: mol_type = unassigned DNA
caggtgcagctgcaggagtcgggcccaggactggtgaagccttcggggaccctgtccctcacctgcattgtctctggtggctccatcatcagtagtgactggtggaattgggtccgccagcccccagggaaggggctggagtggattggagaaatctttcatagtgggaggaccaactacaacccgtccctcaagagtcgagtcaccatatcaatagacaagtccaagaatcagttctccctgaggctgagctctgtgaccgccgcggacacggccgtgtattactgtgcgagagatggttcggggagttactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 102 - protein, 12 aa, Homo sapiens
QSVLYSSNNKNY

SEQ ID NO: 103 - skipped

SEQ ID NO: 104 - protein, 8 aa, Homo sapiens
QQYYSNRS

SEQ ID NO: 105 - protein, 17 aa, Homo sapiens
KSSQSVLYSSNNKNYLA

SEQ ID NO: 106 - protein, 7 aa, Homo sapiens
WASTRES

SEQ ID NO: 107 - protein, 8 aa, Homo sapiens
QQYYSNRS

SEQ ID NO: 108 - protein, 112 aa, Homo sapiens
DIVMTQSPDSLAVSLGERATINCKSSQSVLYSSNNKNYLAWYQQKSGQPPKLLIYWASTRESGVPDRFSGSGSGTDFTLTISSLQTEDVAVYYCQQYYSNRSFGQGTKLEIK

SEQ ID NO: 109 - DNA, 337 bp, Homo sapiens
  source 1..337: mol_type = unassigned DNA
gacatcgtgatgacccagtctccagactccctggctgtgtctctgggcgagagggccaccatcaactgcaagtccagccagagtgttttatacagctccaacaataagaattacttagcttggtaccagcagaaatcaggacagcctcctaagttgctcatttactgggcatctacccgggaatccggggtccctgaccgattcagtggcagcgggtctgggacagatttcactctcaccatcagcagcctgcagactgaagatgtggcagtttattactgtcagcaatattatagtaatcgcagttttggccaggggaccaagctggagatcaaac

SEQ ID NO: 110 - protein, 219 aa, Homo sapiens
DIVMTQSPDSLAVSLGERATINCKSSQSVLYSSNNKNYLAWYQQKSGQPPKLLIYWASTRESGVPDRFSGSGSGTDFTLTISSLQTEDVAVYYCQQYYSNRSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - DNA, 657 bp, Homo sapiens
  source 1..657: mol_type = unassigned DNA
gacatcgtgatgacccagtctccagactccctggctgtgtctctgggcgagagggccaccatcaactgcaagtccagccagagtgttttatacagctccaacaataagaattacttagcttggtaccagcagaaatcaggacagcctcctaagttgctcatttactgggcatctacccgggaatccggggtccctgaccgattcagtggcagcgggtctgggacagatttcactctcaccatcagcagcctgcagactgaagatgtggcagtttattactgtcagcaatattatagtaatcgcagttttggccaggggaccaagctggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 112 - protein, 8 aa, Homo sapiens
GFTFSSYW

SEQ ID NO: 113 - protein, 8 aa, Homo sapiens
IKEDGSEK

SEQ ID NO: 114 - protein, 12 aa, Homo sapiens
ARNRLYSDFLDN

SEQ ID NO: 115 - protein, 5 aa, Homo sapiens
SYWMS

SEQ ID NO: 116 - protein, 17 aa, Homo sapiens
NIKEDGSEKYYVDSVKG

SEQ ID NO: 117 - protein, 10 aa, Homo sapiens
NRLYSDFLDN

SEQ ID NO: 118 - protein, 119 aa, Homo sapiens; identical to SEQ ID NO: 58

SEQ ID NO: 119 - DNA, 358 bp, Homo sapiens; identical to SEQ ID NO: 59
  source 1..358: mol_type = unassigned DNA

SEQ ID NO: 120 - protein, 449 aa, Homo sapiens; identical to SEQ ID NO: 60

SEQ ID NO: 121 - DNA, 1347 bp, Homo sapiens; identical to SEQ ID NO: 61
  source 1..1347: mol_type = unassigned DNA

SEQ ID NO: 122 - protein, 6 aa, Homo sapiens
QGVSSW

SEQ ID NO: 123 - skipped

SEQ ID NO: 124 - protein, 9 aa, Homo sapiens
QQANSIPFT

SEQ ID NO: 125 - protein, 11 aa, Homo sapiens
RASQGVSSWLA

SEQ ID NO: 126 - protein, 7 aa, Homo sapiens
GASSLQS

SEQ ID NO: 127 - protein, 9 aa, Homo sapiens
QQANSIPFT

SEQ ID NO: 128 - protein, 107 aa, Homo sapiens; identical to SEQ ID NO: 68

SEQ ID NO: 129 - DNA, 322 bp, Homo sapiens; identical to SEQ ID NO: 69
  source 1..322: mol_type = unassigned DNA

SEQ ID NO: 130 - protein, 214 aa, Homo sapiens; identical to SEQ ID NO: 70

SEQ ID NO: 131 - DNA, 642 bp, Homo sapiens; identical to SEQ ID NO: 71
  source 1..642: mol_type = unassigned DNA

SEQ ID NO: 132 - protein, 8 aa, Homo sapiens
GFTFRIYG

SEQ ID NO: 133 - protein, 8 aa, Homo sapiens
IWYDGSNK

SEQ ID NO: 134 - protein, 11 aa, Homo sapiens
ARDMDYFGMDV

SEQ ID NO: 135 - protein, 5 aa, Homo sapiens
IYGMH

SEQ ID NO: 136 - protein, 17 aa, Homo sapiens
VIWYDGSNKYYADSVKG

SEQ ID NO: 137 - protein, 9 aa, Homo sapiens
DMDYFGMDV

SEQ ID NO: 138 - protein, 118 aa, Homo sapiens
QVQLVESGGGVVQPGRSLRLSCAASGFTFRIYGMHWVRQAPGKGLEWVAVIWYDGSNKYYADSVKGRFTISRDNSDNTLYLQMNSLRAEDTAVYYCARDMDYFGMDVWGQGTTVTVSS

SEQ ID NO: 139 - DNA, 355 bp, Homo sapiens
  source 1..355: mol_type = unassigned DNA
caggtgcagctggtggagtctgggggaggcgtggtccagcctgggaggtccctgagactctcctgtgcagcgtctggattcaccttccgtatttatggcatgcactgggtccgccaggctccaggcaaggggctggagtgggtggcagttatatggtatgatggaagtaataaatactatgctgactccgtgaagggccgattcaccatctccagagacaattccgacaacacgctgtatctgcaaatgaacagcctgagagccgaggacacggctgtgtattactgtgcgagagatatggactacttcggtatggacgtctggggccaagggaccacggtcaccgtctcctcag

SEQ ID NO: 140 - protein, 448 aa, Homo sapiens
QVQLVESGGGVVQPGRSLRLSCAASGFTFRIYGMHWVRQAPGKGLEWVAVIWYDGSNKYYADSVKGRFTISRDNSDNTLYLQMNSLRAEDTAVYYCARDMDYFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - DNA, 1344 bp, Homo sapiens
  source 1..1344: mol_type = unassigned DNA
caggtgcagctggtggagtctgggggaggcgtggtccagcctgggaggtccctgagactctcctgtgcagcgtctggattcaccttccgtatttatggcatgcactgggtccgccaggctccaggcaaggggctggagtgggtggcagttatatggtatgatggaagtaataaatactatgctgactccgtgaagggccgattcaccatctccagagacaattccgacaacacgctgtatctgcaaatgaacagcctgagagccgaggacacggctgtgtattactgtgcgagagatatggactacttcggtatggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 142 - protein, 6 aa, Homo sapiens
QGIRND

SEQ ID NO: 143 - skipped

SEQ ID NO: 144 - protein, 9 aa, Homo sapiens
LQHNSYPRT

SEQ ID NO: 145 - protein, 11 aa, Homo sapiens
RASQGIRNDLG

SEQ ID NO: 146 - protein, 7 aa, Homo sapiens
AASSLQS

SEQ ID NO: 147 - protein, 9 aa, Homo sapiens
LQHNSYPRT

SEQ ID NO: 148 - protein, 107 aa, Homo sapiens
DLQMTQSPSSLSASVGDRVTITCRASQGIRNDLGWYQQKPGKAPKRLIYAASSLQSGVPSRFSGSGSGTEFTLTISSLQPEDFATYYCLQHNSYPRTFGQGTKVEIK

SEQ ID NO: 149 - DNA, 322 bp, Homo sapiens
  source 1..322: mol_type = unassigned DNA
gacctccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttaccctcggacgttcggccaagggaccaaggtggaaatcaaac

SEQ ID NO: 150 - protein, 214 aa, Homo sapiens
DLQMTQSPSSLSASVGDRVTITCRASQGIRNDLGWYQQKPGKAPKRLIYAASSLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - DNA, 642 bp, Homo sapiens
  source 1..642: mol_type = unassigned DNA
gacctccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttaccctcggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 152 - protein, 9 aa, Homo sapiens
GGSISSSDW

SEQ ID NO: 153 - protein, 7 aa, Homo sapiens
IFHSGNT

SEQ ID NO: 154 - protein, 8 aa, Homo sapiens
VRDGSGSY

SEQ ID NO: 155 - protein, 6 aa, Homo sapiens
SSDWWS

SEQ ID NO: 156 - protein, 16 aa, Homo sapiens
EIFHSGNTNYNPSLKS

SEQ ID NO: 157 - protein, 6 aa, Homo sapiens
DGSGSY

SEQ ID NO: 158 - protein, 115 aa, Homo sapiens
QVQLQESGPGLVKPSGTLSLTCAVSGGSISSSDWWSWVRQPPGKGLEWIGEIFHSGNTNYNPSLKSRVTISVDKSKNQISLRLNSVTAADTAVYYCVRDGSGSYWGQGTLVTVSS

SEQ ID NO: 159 - DNA, 346 bp, Homo sapiens
  source 1..346: mol_type = unassigned DNA
caggtgcagctgcaggagtcgggcccaggactggtgaagccttcggggaccctgtccctcacctgcgctgtctctggtggctccatcagcagtagtgactggtggagttgggtccgccagcccccagggaaggggctggagtggattggggaaatctttcatagtgggaacaccaactacaacccgtccctcaagagtcgagtcaccatatcagtagacaagtccaagaaccagatctccctgaggctgaactctgtgaccgccgcggacacggccgtgtattactgtgtgagagatggttcggggagttactggggccagggaaccctggtcaccgtctcctcag

SEQ ID NO: 160 - protein, 445 aa, Homo sapiens
QVQLQESGPGLVKPSGTLSLTCAVSGGSISSSDWWSWVRQPPGKGLEWIGEIFHSGNTNYNPSLKSRVTISVDKSKNQISLRLNSVTAADTAVYYCVRDGSGSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - DNA, 1335 bp, Homo sapiens
  source 1..1335: mol_type = unassigned DNA
caggtgcagctgcaggagtcgggcccaggactggtgaagccttcggggaccctgtccctcacctgcgctgtctctggtggctccatcagcagtagtgactggtggagttgggtccgccagcccccagggaaggggctggagtggattggggaaatctttcatagtgggaacaccaactacaacccgtccctcaagagtcgagtcaccatatcagtagacaagtccaagaaccagatctccctgaggctgaactctgtgaccgccgcggacacggccgtgtattactgtgtgagagatggttcggggagttactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 162 - protein, 12 aa, Homo sapiens
QSVLYSSNNKNY

SEQ ID NO: 163 - skipped

SEQ ID NO: 164 - protein, 8 aa, Homo sapiens
QQYYSTRS

SEQ ID NO: 165 - protein, 17 aa, Homo sapiens
KSSQSVLYSSNNKNYLA

SEQ ID NO: 166 - protein, 7 aa, Homo sapiens
WASTRES

SEQ ID NO: 167 - protein, 8 aa, Homo sapiens
QQYYSTRS

SEQ ID NO: 168 - protein, 112 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTRSFGQGTKLEIK

SEQ ID NO: 169 - DNA, 337 bp, Homo sapiens
  source 1..337: mol_type = unassigned DNA
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaaactgctcatttactgggcatctacccgggaatccggggtccctgaccgattcagtggcagcgggtctgggacagatttcactctcaccatcagcagcctgcaggctgaagatgtggcagtttattactgtcagcaatattatagtactcgcagttttggccaggggaccaagctggagatcaaac

SEQ ID NO: 170 - protein, 219 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTRSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - DNA, 657 bp, Homo sapiens
  source 1..657: mol_type = unassigned DNA
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaaactgctcatttactgggcatctacccgggaatccggggtccctgaccgattcagtggcagcgggtctgggacagatttcactctcaccatcagcagcctgcaggctgaagatgtggcagtttattactgtcagcaatattatagtactcgcagttttggccaggggaccaagctggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 172 - protein, 10 aa, Homo sapiens
GGSISSSSYY

SEQ ID NO: 173 - protein, 7 aa, Homo sapiens
IYSTGYT

SEQ ID NO: 174 - protein, 13 aa, Homo sapiens
AISTAAGPEYFHR

SEQ ID NO: 175 - protein, 7 aa, Homo sapiens
SSSYYCG

SEQ ID NO: 176 - protein, 16 aa, Homo sapiens
SIYSTGYTYYNPSLKS

SEQ ID NO: 177 - protein, 11 aa, Homo sapiens
STAAGPEYFHR

SEQ ID NO: 178 - protein, 120 aa, Homo sapiens
QLQESGPGLVKPSETLSLTCTVSGGSISSSSYYCGWIRQPPGKGLDWIGSIYSTGYTYYNPSLKSRVTISIDTSKNQFSCLILTSVTAADTAVYYCAISTAAGPEYFHRWGQGTLVTVSS

SEQ ID NO: 179 - DNA, 361 bp, Homo sapiens
  source 1..361: mol_type = unassigned DNA
cagctgcaggagtcgggcccaggcctggtgaagccttcggagaccctgtccctcacctgcactgtctctggtggctccatcagcagtagtagttattactgcggctggatccgccagccccctgggaaggggctggactggattgggagtatctattctactgggtacacctactacaacccgtccctcaagagtcgagtcaccatttccatagacacgtccaagaaccagttctcatgcctgatactgacctctgtgaccgccgcagacacggctgtgtattactgtgcgataagtacagcagctggccctgaatacttccatcgctggggccagggcaccctggtcaccgtctcctcag

SEQ ID NO: 180 - protein, 450 aa, Homo sapiens
QLQESGPGLVKPSETLSLTCTVSGGSISSSSYYCGWIRQPPGKGLDWIGSIYSTGYTYYNPSLKSRVTISIDTSKNQFSCLILTSVTAADTAVYYCAISTAAGPEYFHRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - DNA, 1350 bp, Homo sapiens
  source 1..1350: mol_type = unassigned DNA
cagctgcaggagtcgggcccaggcctggtgaagccttcggagaccctgtccctcacctgcactgtctctggtggctccatcagcagtagtagttattactgcggctggatccgccagccccctgggaaggggctggactggattgggagtatctattctactgggtacacctactacaacccgtccctcaagagtcgagtcaccatttccatagacacgtccaagaaccagttctcatgcctgatactgacctctgtgaccgccgcagacacggctgtgtattactgtgcgataagtacagcagctggccctgaatacttccatcgctggggccagggcaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 182 - protein, 12 aa, Homo sapiens
QSVLYSSNSKNF

SEQ ID NO: 183 - skipped

SEQ ID NO: 184 - protein, 9 aa, Homo sapiens
QQYYSTPRT

SEQ ID NO: 185 - protein, 17 aa, Homo sapiens
KSSQSVLYSSNSKNFLA

SEQ ID NO: 186 - protein, 7 aa, Homo sapiens
WASTRGS

SEQ ID NO: 187 - protein, 9 aa, Homo sapiens
QQYYSTPRT

SEQ ID NO: 188 - protein, 113 aa, Homo sapiens
DIVMTQSPDSLAVSLGERATINCKSSQSVLYSSNSKNFLAWYQQKPGQPPKLFIYWASTRGSGVPDRISGSGSGTDFNLTISSLQAEDVAVYYCQQYYSTPRTFGQGTKVEIK

SEQ ID NO: 189 - DNA, 340 bp, Homo sapiens
  source 1..340: mol_type = unassigned DNA
gacatcgtgatgacccagtctccagactccctggctgtgtctctgggcgagagggccaccatcaactgcaagtccagccagagtgttttatacagctccaacagtaagaacttcttagcttggtaccagcagaaaccgggacagcctcctaagctgttcatttactgggcatctacccggggatccggggtccctgaccgaatcagtggcagcgggtctgggacagatttcaatctcaccatcagcagcctgcaggctgaagatgtggcagtttattactgtcaacaatattatagtactcctcggacgttcggccaagggaccaaggtggagatcaaac

SEQ ID NO: 190 - protein, 220 aa, Homo sapiens
DIVMTQSPDSLAVSLGERATINCKSSQSVLYSSNSKNFLAWYQQKPGQPPKLFIYWASTRGSGVPDRISGSGSGTDFNLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - DNA, 660 bp, Homo sapiens
  source 1..660: mol_type = unassigned DNA
gacatcgtgatgacccagtctccagactccctggctgtgtctctgggcgagagggccaccatcaactgcaagtccagccagagtgttttatacagctccaacagtaagaacttcttagcttggtaccagcagaaaccgggacagcctcctaagctgttcatttactgggcatctacccggggatccggggtccctgaccgaatcagtggcagcgggtctgggacagatttcaatctcaccatcagcagcctgcaggctgaagatgtggcagtttattactgtcaacaatattatagtactcctcggacgttcggccaagggaccaaggtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 192 - DNA, 981 bp, Homo sapiens
  source 1..981: mol_type = unassigned DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 193 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 194 - DNA, 981 bp, Homo sapiens
  source 1..981: mol_type = unassigned DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccgtgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcgtgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacgcagaagagcctctccctgtctctgggtaaa

SEQ ID NO: 195 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 196 - DNA, 981 bp, Homo sapiens
  source 1..981: mol_type = unassigned DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctcaccgtggacaagagcaggtggcaggaggggaacgtcttctcatgctccgtgatgcatgaggctctgcacaaccactacacgcagaagagcctctccctgtctctgggtaaa

SEQ ID NO: 197 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 198 - DNA, 981 bp, Homo sapiens
  source 1..981: mol_type = unassigned DNA
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctgaatttgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 199 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 200 - DNA, 981 bp, Homo sapiens
  source 1..981: mol_type = unassigned DNA
gcctccaccaagggacctagcgtgttccctctcgccccctgttccaggtccacaagcgagtccaccgctgccctcggctgtctggtgaaagactactttcccgagcccgtgaccgtctcctggaatagcggagccctgacctccggcgtgcacacatttcccgccgtgctgcagagcagcggactgtatagcctgagcagcgtggtgaccgtgcccagctccagcctcggcaccaaaacctacacctgcaacgtggaccacaagccctccaacaccaaggtggacaagcgggtggagagcaagtacggccccccttgccctccttgtcctgcccctgagttcgagggaggaccctccgtgttcctgtttccccccaaacccaaggacaccctgatgatctcccggacacccgaggtgacctgtgtggtcgtggacgtcagccaggaggaccccgaggtgcagttcaactggtatgtggacggcgtggaggtgcacaatgccaaaaccaagcccagggaggagcagttcaattccacctacagggtggtgagcgtgctgaccgtcctgcatcaggattggctgaacggcaaggagtacaagtgcaaggtgtccaacaagggactgcccagctccatcgagaagaccatcagcaaggctaagggccagccgagggagccccaggtgtataccctgcctcctagccaggaagagatgaccaagaaccaagtgtccctgacctgcctggtgaagggattctacccctccgacatcgccgtggagtgggagagcaatggccagcccgagaacaactacaaaacaacccctcccgtgctcgatagcgacggcagcttctttctctacagccggctgacagtggacaagagcaggtggcaggagggcaacgtgttctcctgttccgtgatgcacgaggccctgcacaatcactacacccagaagagcctctccctgtccctgggcaag

SEQ ID NO: 201 - DNA, 981 bp, Homo sapiens
  source 1..981: mol_type = unassigned DNA
gccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 202 - DNA, 981 bp, Homo sapiens
  source 1..981: mol_type = unassigned DNA
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctccagttgcggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 203 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 204 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtggagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 205 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - DNA, 321 bp, Homo sapiens
  source 1..321: mol_type = unassigned DNA
cgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 207 - protein, 107 aa, Homo sapiens [sequence omitted]

SEQ ID NO: 208 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggagagcaaggacagcacctacagcctcagcagcaccctgacgctgagcaaagcagactacgagaaacacaaagtctacgccggcgaagtcacccatcagggcctgagctcgcccgtcacaaagagcttcaacaggggagagtgt

SEQ ID NO: 209 - protein, 107 aa, Homo sapiens
RTVAAPSVFIFPPSDEQLKSGTASVVCLLNNFYPREAKVQWKVDNALQSGNSQESVTEQESKDSTYSLSSTLTLSKADYEKHKVYAGEVTHQGLSSPVTKSFNRGEC

SEQ ID NO: 210 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagcggaaggtggataacgccctccaatcgggtaactcccaggagagtgtcacagagcaggagagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 211 - protein, 107 aa, Homo sapiens
RTVAAPSVFIFPPSDEQLKSGTASVVCLLNNFYPREAKVQRKVDNALQSGNSQESVTEQESKDSTYSLSSTLTLSKADYEKHKVYACEVTHQGLSSPVTKSFNRGEC

SEQ ID NO: 212 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 213 - protein, 107 aa, Homo sapiens
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 214 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcaacaccctgacgctgagcaaagcagactacgagaaacacaaagtctacgcctgcgaagtcacccatcagggcctgagctcgcccgtcacaaagagcttcaacaggggagagtgc

SEQ ID NO: 215 - protein, 107 aa, Homo sapiens
RTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 216 - DNA, 312 bp, Homo sapiens
  source 1..312: mol_type = unassigned DNA
cccaaggccaaccccacggtcactctgttcccgccctcctctgaggagctccaagccaacaaggccacactagtgtgtctgatcagtgacttctacccgggagctgtgacagtggcttggaaggcagatggcagccccgtcaaggcgggagtggagacgaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 217 - protein, 104 aa, Homo sapiens
PKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 218 - DNA, 318 bp, Homo sapiens
  source 1..318: mol_type = unassigned DNA
ggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 219 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 220 - DNA, 318 bp, Homo sapiens; identical to SEQ ID NO: 218
  source 1..318: mol_type = unassigned DNA

SEQ ID NO: 221 - DNA, 318 bp, Homo sapiens
  source 1..318: mol_type = unassigned DNA
ggccagcctaaggccgctccttctgtgaccctgttccccccatcctccgaggaactgcaggctaacaaggccaccctcgtgtgcctgatcagcgacttctaccctggcgccgtgaccgtggcctggaaggctgatagctctcctgtgaaggccggcgtggaaaccaccaccccttccaagcagtccaacaacaaatacgccgcctcctcctacctgtccctgacccctgagcagtggaagtcccaccggtcctacagctgccaagtgacccacgagggctccaccgtggaaaagaccgtggctcctaccgagtgctcc

SEQ ID NO: 222 - DNA, 318 bp, Homo sapiens
  source 1..318: mol_type = unassigned DNA
ggccagcctaaagctgcccccagcgtcaccctgtttcctccctccagcgaggagctccaggccaacaaggccaccctcgtgtgcctgatctccgacttctatcccggcgctgtgaccgtggcttggaaagccgactccagccctgtcaaagccggcgtggagaccaccacaccctccaagcagtccaacaacaagtacgccgcctccagctatctctccctgacccctgagcagtggaagtcccaccggtcctactcctgtcaggtgacccacgagggctccaccgtggaaaagaccgtcgcccccaccgagtgctcc

SEQ ID NO: 223 - protein, 106 aa, Homo sapiens; identical to SEQ ID NO: 219

SEQ ID NO: 224 - DNA, 318 bp, Homo sapiens
  source 1..318: mol_type = unassigned DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 225 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 226 - DNA, 312 bp, Homo sapiens
  source 1..312: mol_type = unassigned DNA
cccaaggctgccccctcggtcactctgttcccaccctcctctgaggagcttcaagccaacaaggccacactggtgtgtctcataagtgacttctacccgggagccgtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagttgcccctacggaatgttca

SEQ ID NO: 227 - protein, 104 aa, Homo sapiens
PKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 228 - DNA, 318 bp, Homo sapiens
  source 1..318: mol_type = unassigned DNA
ggtcagcccaaggctgccccctcggtcactctgttcccaccctcctctgaggagcttcaagccaacaaggccacactggtgtgtctcataagtgacttctacccggggccagtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacggaatgttca

SEQ ID NO: 229 - protein, 106 aa, Homo sapiens
GQPKAAPSVTLFPPSSEELQANKATLVCLISDFYPGPVTVAWKADSSPVKAGVETTTPSKQSNNKYAASSYLSLTPEQWKSHKSYSCQVTHEGSTVEKTVAPTECS

SEQ ID NO: 230 - DNA, 318 bp, Homo sapiens
  source 1..318: mol_type = unassigned DNA
ggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 231 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 232 - DNA, 318 bp, Homo sapiens
  source 1..318: mol_type = unassigned DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 233 - protein, 106 aa, Homo sapiens; identical to SEQ ID NO: 225

SEQ ID NO: 234 - DNA, 318 bp, Homo sapiens
  source 1..318: mol_type = unassigned DNA
ggtcagcccaaggctgccccatcggtcactctgttcccgccctcctctgaggagcttcaagccaacaaggccacactggtgtgcctgatcagtgacttctacccgggagctgtgaaagtggcctggaaggcagatggcagccccgtcaacacgggagtggagaccaccacaccctccaaacagagcaacaacaagtacgcggccagcagctacctgagcctgacgcctgagcagtggaagtcccacagaagctacagctgccaggtcacgcatgaagggagcaccgtggagaagacagtggcccctgcagaatgttca

SEQ ID NO: 235 - protein, 106 aa, Homo sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 236 - DNA, 318 bp, Homo sapiens
  source 1..318: mol_type = unassigned DNA
ggtcagcccaaggctgccccatcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 237 - protein, 106 aa, Homo sapiens
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 238 - protein, 8 aa, Homo sapiens
GFTFSDYY

SEQ ID NO: 239 - protein, 8 aa, Homo sapiens
ISTSGSTI

SEQ ID NO: 240 - protein, 16 aa, Homo sapiens
ARGITGTNFYHYGLGV

SEQ ID NO: 241 - protein, 5 aa, Homo sapiens
DYYMS

SEQ ID NO: 242 - protein, 17 aa, Homo sapiens
YISTSGSTIYYADSVKG

SEQ ID NO: 243 - protein, 14 aa, Homo sapiens
GITGTNFYHYGLGV

SEQ ID NO: 244 - protein, 123 aa, Homo sapiens
QVQLVESGGGLVKPGGSLRLSCAASGFTFSDYYMSWIRQVPGKGLEWVSYISTSGSTIYYADSVKGRFTISRDNAKNSLYLQMNSLRAEDAAVYHCARGITGTNFYHYGLGVWGQGTTVTVSS

SEQ ID NO: 245 - DNA, 370 bp, Homo sapiens
  source 1..370: mol_type = unassigned DNA
caggtgcagctggtggagtctgggggaggcttggtcaagcctggagggtccctgagactctcctgtgcagcctctggattcaccttcagtgactactacatgagctggatccgccaggttccagggaaggggctggagtgggtttcatacattagtactagtggtagtaccatatactacgcagactctgtgaagggccgattcaccatctccagggacaacgccaagaactcactgtatctacaaatgaacagcctgagagccgaggacgcggccgtgtatcactgtgcgagaggtataactggaactaacttctaccactacggtttgggcgtctggggccaagggaccacggtcaccgtctcctcag

SEQ ID NO: 246 - protein, 453 aa, Homo sapiens
QVQLVESGGGLVKPGGSLRLSCAASGFTFSDYYMSWIRQVPGKGLEWVSYISTSGSTIYYADSVKGRFTISRDNAKNSLYLQMNSLRAEDAAVYHCARGITGTNFYHYGLGVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - DNA, 1359 bp, Homo sapiens
  source 1..1359: mol_type = unassigned DNA
caggtgcagctggtggagtctgggggaggcttggtcaagcctggagggtccctgagactctcctgtgcagcctctggattcaccttcagtgactactacatgagctggatccgccaggttccagggaaggggctggagtgggtttcatacattagtactagtggtagtaccatatactacgcagactctgtgaagggccgattcaccatctccagggacaacgccaagaactcactgtatctacaaatgaacagcctgagagccgaggacgcggccgtgtatcactgtgcgagaggtataactggaactaacttctaccactacggtttgggcgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 248 - protein, 6 aa, Homo sapiens
QGINSW

SEQ ID NO: 249 - skipped

SEQ ID NO: 250 - protein, 9 aa, Homo sapiens
QQVNSFPLT

SEQ ID NO: 251 - protein, 11 aa, Homo sapiens
RASQGINSWLA

SEQ ID NO: 252 - protein, 7 aa, Homo sapiens
AASTLQS

SEQ ID NO: 253 - protein, 9 aa, Homo sapiens
QQVNSFPLT

SEQ ID NO: 254 - protein, 107 aa, Homo sapiens
DIQMTQSPSSVSASVGDRVTITCRASQGINSWLAWYQQKPGKAPKLLIYAASTLQSGVPSRFSGSGSGADFTLTISSLQPEDFATYYCQQVNSFPLTFGGGTKVEIK

SEQ ID NO: 255 - DNA, 322 bp, Homo sapiens
  source 1..322: mol_type = unassigned DNA
gacatccagatgacccagtctccatcttccgtgtctgcatctgtaggagacagagtcaccatcacttgtcgggcgagtcagggtattaacagctggttagcctggtatcagcagaaaccagggaaagcccctaagctcctgatctatgctgcatccactttgcaaagtggggtcccatcaaggttcagcggcagtgggtctggggcagatttcactctcaccatcagcagcctgcagcctgaagattttgcaacttactattgtcaacaggttaacagtttcccgctcactttcggcggagggaccaaggtggagatcaaac

SEQ ID NO: 256 - protein, 214 aa, Homo sapiens
DIQMTQSPSSVSASVGDRVTITCRASQGINSWLAWYQQKPGKAPKLLIYAASTLQSGVPSRFSGSGSGADFTLTISSLQPEDFATYYCQQVNSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - DNA, 642 bp, Homo sapiens
  source 1..642: mol_type = unassigned DNA
gacatccagatgacccagtctccatcttccgtgtctgcatctgtaggagacagagtcaccatcacttgtcgggcgagtcagggtattaacagctggttagcctggtatcagcagaaaccagggaaagcccctaagctcctgatctatgctgcatccactttgcaaagtggggtcccatcaaggttcagcggcagtgggtctggggcagatttcactctcaccatcagcagcctgcagcctgaagattttgcaacttactattgtcaacaggttaacagtttcccgctcactttcggcggagggaccaaggtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 258 - protein, 8 aa, Homo sapiens
GFTFSYYA

SEQ ID NO: 259 - protein, 8 aa, Homo sapiens
ISGGGGNT

SEQ ID NO: 260 - protein, 16 aa, Homo sapiens
AKDRMKQLVRAYYFDY

SEQ ID NO: 261 - protein, 5 aa, Homo sapiens
YYAMS

SEQ ID NO: 262 - protein, 17 aa, Homo sapiens
TISGGGGNTHYADSVKG

SEQ ID NO: 263 - protein, 14 aa, Homo sapiens
DRMKQLVRAYYFDY

SEQ ID NO: 264 - protein, 123 aa, Homo sapiens
EVPLVESGGGLVQPGGSLRLSCAASGFTFSYYAMSWVRQAPGKGLDWVSTISGGGGNTHYADSVKGRFTISRDNSKNTLYLHMNSLRAEDTAVYYCAKDRMKQLVRAYYFDYWGQGTLVTVSS

SEQ ID NO: 265 - DNA, 370 bp, Homo sapiens
  source 1..370: mol_type = unassigned DNA
gaggtgccgctggtggagtctgggggaggcttggtacagcctggggggtccctgagactctcctgtgcagcctctggattcacgtttagctactatgccatgagctgggtccgtcaggctccagggaaggggctggactgggtctcaactattagtggtggtggtggtaacacacactacgcagactccgtgaagggccgattcactatatccagagacaattccaagaacacgctgtatctgcacatgaacagcctgagagccgaagacacggccgtctattactgtgcgaaggatcggatgaaacagctcgtccgggcctactactttgactactggggccagggaaccctggtcaccgtctcctcag

SEQ ID NO: 266 - protein, 453 aa, Homo sapiens
EVPLVESGGGLVQPGGSLRLSCAASGFTFSYYAMSWVRQAPGKGLDWVSTISGGGGNTHYADSVKGRFTISRDNSKNTLYLHMNSLRAEDTAVYYCAKDRMKQLVRAYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 267 - DNA, 1359 bp, Homo sapiens
  source 1..1359: mol_type = unassigned DNA
gaggtgccgctggtggagtctgggggaggcttggtacagcctggggggtccctgagactctcctgtgcagcctctggattcacgtttagctactatgccatgagctgggtccgtcaggctccagggaaggggctggactgggtctcaactattagtggtggtggtggtaacacacactacgcagactccgtgaagggccgattcactatatccagagacaattccaagaacacgctgtatctgcacatgaacagcctgagagccgaagacacggccgtctattactgtgcgaaggatcggatgaaacagctcgtccgggcctactactttgactactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 268 - protein, 6 aa, Homo sapiens
QDISTY

SEQ ID NO: 269 - skipped

SEQ ID NO: 270 - protein, 9 aa, Homo sapiens
QQLHTDPIT

SEQ ID NO: 271 - protein, 11 aa, Homo sapiens
WASQDISTYLG

SEQ ID NO: 272 - protein, 7 aa, Homo sapiens
GTSSLQS

SEQ ID NO: 273 - protein, 9 aa, Homo sapiens
QQLHTDPIT

SEQ ID NO: 274 - protein, 107 aa, Homo sapiens
DIQLTQSPSFLSASVGDRVTITCWASQDISTYLGWYQQKPGKAPKLLIYGTSSLQSGVPSRFSGSGSGTEFTLTISSLQPEDFATYYCQQLHTDPITFGQGTRLEIK

SEQ ID NO: 275 - DNA, 322 bp, Homo sapiens
  source 1..322: mol_type = unassigned DNA
gacatccagttgacccagtctccatccttcctgtctgcatctgtaggagacagagtcaccatcacttgctgggccagtcaggacattagcacttatttaggctggtatcagcaaaaaccagggaaagcccctaagctcctgatctatggtacatccagtttgcaaagtggggtcccatcaaggttcagcggcagtggatctgggacagaattcactctcacaatcagcagcctgcagcctgaagattttgcaacttattactgtcaacagcttcatactgacccgatcaccttcggccaagggacacgactggagatcaaac

SEQ ID NO: 276 - protein, 214 aa, Homo sapiens
DIQLTQSPSFLSASVGDRVTITCWASQDISTYLGWYQQKPGKAPKLLIYGTSSLQSGVPSRFSGSGSGTEFTLTISSLQPEDFATYYCQQLHTDPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 277 - DNA, 642 bp, Homo sapiens
  source 1..642: mol_type = unassigned DNA
gacatccagttgacccagtctccatccttcctgtctgcatctgtaggagacagagtcaccatcacttgctgggccagtcaggacattagcacttatttaggctggtatcagcaaaaaccagggaaagcccctaagctcctgatctatggtacatccagtttgcaaagtggggtcccatcaaggttcagcggcagtggatctgggacagaattcactctcacaatcagcagcctgcagcctgaagattttgcaacttattactgtcaacagcttcatactgacccgatcaccttcggccaagggacacgactggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 278 - protein, 8 aa, Homo sapiens
GFTFSSYW

SEQ ID NO: 279 - protein, 8 aa, Homo sapiens
IKQDGSEK

SEQ ID NO: 280 - protein, 12 aa, Homo sapiens
ARVRQWSDYSDY

SEQ ID NO: 281 - protein, 5 aa, Homo sapiens
SYWMN

SEQ ID NO: 282 - protein, 17 aa, Homo sapiens
NIKQDGSEKYYVDSVKG

SEQ ID NO: 283 - protein, 10 aa, Homo sapiens
VRQWSDYSDY

SEQ ID NO: 284 - protein, 119 aa, Homo sapiens
EVHLVESGGGLVQPGGSLRLSCAASGFTFSSYWMNWVRQAPGKGLEWVANIKQDGSEKYYVDSVKGRFTVSRDNAKNSLYLQMNSLRAEDTAVYYCARVRQWSDYSDYWGQGTPVTVSS

SEQ ID NO: 285 - DNA, 358 bp, Homo sapiens
  source 1..358: mol_type = unassigned DNA
gaggtgcacctggtggagtctgggggaggcttggtccagcctggggggtccctgagactctcctgtgcagcctctggattcacctttagtagctattggatgaactgggtccgccaggctccagggaaggggctggagtgggtggccaacataaagcaagatggaagtgagaaatactatgtggactctgtgaagggccgcttcaccgtctccagagacaacgccaagaactcactgtatctgcaaatgaacagcctgagagccgaggacacggctgtgtattactgtgcgagagttcgacaatggtccgactactctgactactggggccagggaaccccggtcaccgtctcctcag

SEQ ID NO: 286 - protein, 449 aa, Homo sapiens
EVHLVESGGGLVQPGGSLRLSCAASGFTFSSYWMNWVRQAPGKGLEWVANIKQDGSEKYYVDSVKGRFTVSRDNAKNSLYLQMNSLRAEDTAVYYCARVRQWSDYSDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 287 - DNA, 1347 bp, Homo sapiens
  source 1..1347: mol_type = unassigned DNA
gaggtgcacctggtggagtctgggggaggcttggtccagcctggggggtccctgagactctcctgtgcagcctctggattcacctttagtagctattggatgaactgggtccgccaggctccagggaaggggctggagtgggtggccaacataaagcaagatggaagtgagaaatactatgtggactctgtgaagggccgcttcaccgtctccagagacaacgccaagaactcactgtatctgcaaatgaacagcctgagagccgaggacacggctgtgtattactgtgcgagagttcgacaatggtccgactactctgactactggggccagggaaccccggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 288 - protein, 6 aa, Homo sapiens
QGISSW

SEQ ID NO: 289 - skipped

SEQ ID NO: 290 - protein, 9 aa, Homo sapiens
QQANSFPFT

SEQ ID NO: 291 - protein, 11 aa, Homo sapiens
RASQGISSWLA

SEQ ID NO: 292 - protein, 7 aa, Homo sapiens
AASSLQS

SEQ ID NO: 293 - protein, 9 aa, Homo sapiens
QQANSFPFT

SEQ ID NO: 294 - protein, 107 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFPFTFGPGTKVDIK

SEQ ID NO: 295 - DNA, 322 bp, Homo sapiens
  source 1..322: mol_type = unassigned DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccattcactttcggccctgggaccaaagtggatatcaaac

SEQ ID NO: 296 - protein, 214 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 297 - DNA, 642 bp, Homo sapiens
  source 1..642: mol_type = unassigned DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 298 - protein, 214 aa, Homo sapiens
DIQMTQSPSSLSASVGDRVTITCRASQSISSYLNWYQQKPGKAPKLLIYYASSLQSGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 299 - protein, 453 aa, Homo sapiens
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 300 - protein, 347 aa, Homo sapiens
DIQMTQSPSSLSASVGDRVTITCRASQSISSYLNWYQQKPGKAPKLLIYVASSLQSGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 301 - protein, 133 aa, Homo sapiens [sequence omitted]

SEQ ID NO: 302 - protein, 586 aa, Homo sapiens
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 303 - protein, 14 aa, Homo sapiens
APTSTQLQLELLLD

SEQ ID NO: 304 - protein, 11 aa, Homo sapiens
TQLQLEHLLLD

SEQ ID NO: 305 - protein, 17 aa, Homo sapiens
APTSKKTQLQLEHLLLD

SEQ ID NO: 306 - protein, 19 aa, Homo sapiens
PTSSSTKKTQLQLEHLLLD

SEQ ID NO: 307 - protein, 18 aa, Homo sapiens
TSSSTKKTQLQLEHLLLD

SEQ ID NO: 308 - protein, 17 aa, Homo sapiens
SSSTKKTQLQLEHLLLD

SEQ ID NO: 309 - protein, 16 aa, Homo sapiens
SSTKKTQLQLEHLLLD

SEQ ID NO: 310 - protein, 15 aa, Homo sapiens
STKKTQLQLEHLLLD

SEQ ID NO: 311 - protein, 14 aa, Homo sapiens
TKKTQLQLEHLLLD

SEQ ID NO: 312 - protein, 13 aa, Homo sapiens
KKTQLQLEHLLLD

SEQ ID NO: 313 - protein, 12 aa, Homo sapiens
KTQLQLEHLLLD

SEQ ID NO: 314 - protein, 19 aa, Homo sapiens
APTSSSTKTQLQLEHLLLD

SEQ ID NO: 315 - protein, 18 aa, Homo sapiens
APTSSSTTQLQLEHLLLD

SEQ ID NO: 316 - protein, 17 aa, Homo sapiens
APTSSSTQLQLEHLLLD

SEQ ID NO: 317 - protein, 16 aa, Homo sapiens
APTSSTQLQLEHLLLD

SEQ ID NO: 318 - protein, 14 aa, Homo sapiens
APTTQLQLEHLLLD

SEQ ID NO: 319 - protein, 13 aa, Homo sapiens
APTQLQLEHLLLD

SEQ ID NO: 320 - protein, 12 aa, Homo sapiens
ATQLQLEHLLLD

SEQ ID NO: 321 - protein, 15 aa, Homo sapiens
ATKKTQLQLEHLLLD

SEQ ID NO: 322 - protein, 15 aa, Homo sapiens
APKKTQLQLEHLLLD

SEQ ID NO: 323 - protein, 15 aa, Homo sapiens
APTKTQLQLEHLLLD

SEQ ID NO: 324 - protein, 113 aa, Homo sapiens
LQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 325 - protein, 290 aa, Homo sapiens
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEET

SEQ ID NO: 326 - protein, 226 aa, Homo sapiens
FTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHHHHHH

SEQ ID NO: 327 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 328 - protein, 525 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 329 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 330 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 331 - protein, 133 aa, Homo sapiens
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 332 - protein, 133 aa, Homo sapiens [sequence omitted]

SEQ ID NO: 333 - protein, 127 aa, Homo sapiens [sequence omitted]

SEQ ID NO: 334 - protein, 171 aa, Homo sapiens
CDLPQNHGLLSRNTLVLLHQMRRISPFLCLKDRRDFRFPQEMVKGSQLQKAHVMSVLHEMLQQIFSLFHTERSSAAWNMTLLDQLHTELHQQLQHLETCLLQVVGEGESAGAISSPALTLRRYFQGIRVYLKEKKYSDCAWEVVRMEIMKSLFLSTNMQERLRSKDRDLGS

SEQ ID NO: 335 - protein, 177 aa, Homo sapiens
GPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 336 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 337 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 338 - protein, 103 aa, Homo sapiens
TPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 339 - protein, 77 aa, Homo sapiens
VPLSRTVRCTCISISNQPVNPRSLEKLEIIPASQFCPRVEIIATMKKKGEKRCLNPESKAIKNLLKAVSKERSKRSP

SEQ ID NO: 340 - protein, 330 aa, Homo sapiens [sequence omitted]

SEQ ID NO: 341 - DNA, 996 bp, Homo sapiens
  source 1..996: mol_type = unassigned DNA
gccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 342 - protein, 450 aa, Homo sapiens
EVQLVESGGGLVQPGRSLRLSCAASGFTFDDYAMHWVRQAPGKGLEWVSGISWIRTGIGYADSVKGRFTISRDNAKNSLYLQMNSLRAEDTALYYCAKDMKGSGTYGGWFDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 343 - protein, 8 aa, Homo sapiens
GFTFSNYA

SEQ ID NO: 344 - protein, 8 aa, Homo sapiens
ISFSGGTT

SEQ ID NO: 345 - protein, 14 aa, Homo sapiens
AKDEAPAGATFFDS

SEQ ID NO: 346 - protein, 5 aa, Homo sapiens
NYAMS

SEQ ID NO: 347 - protein, 17 aa, Homo sapiens
AISFSGGTTYYADSVKG

SEQ ID NO: 348 - protein, 12 aa, Homo sapiens
DEAPAGATFFDS

SEQ ID NO: 349 - protein, 121 aa, Homo sapiens
EVQLAESGGGLVQPGGSLRLSCAASGFTFSNYAMSWVRQTPGKGLEWVSAISFSGGTTYYADSVKGRFTISRDNSKNTLYLHMNSLRADDTAVYYCAKDEAPAGATFFDSWGQGTLVTVSS

SEQ ID NO: 350 - DNA, 364 bp, Homo sapiens
  source 1..364: mol_type = unassigned DNA
gaagtgcaactggcggagtctgggggaggcttggtacagccgggggggtccctgagactctcctgtgcagcctctggattcacctttagcaactatgccatgagttgggtccgccagactccaggaaaggggctggagtgggtctcagctattagttttagtggtggtactacatactacgctgactccgtgaagggccggttcaccatctccagagacaattccaagaacacgctgtatttgcacatgaacagcctgagagccgatgacacggccgtatattactgtgcgaaagatgaggcaccagctggcgcaaccttctttgactcctggggccagggaacgctggtcaccgtctcctcag

SEQ ID NO: 351 - protein, 448 aa, Homo sapiens
EVQLAESGGGLVQPGGSLRLSCAASGFTFSNYAMSWVRQTPGKGLEWVSAISFSGGTTYYADSVKGRFTISRDNSKNTLYLHMNSLRADDTAVYYCAKDEAPAGATFFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 352 - DNA, 1344 bp, Homo sapiens
  source 1..1344: mol_type = unassigned DNA
gaagtgcaactggcggagtctgggggaggcttggtacagccgggggggtccctgagactctcctgtgcagcctctggattcacctttagcaactatgccatgagttgggtccgccagactccaggaaaggggctggagtgggtctcagctattagttttagtggtggtactacatactacgctgactccgtgaagggccggttcaccatctccagagacaattccaagaacacgctgtatttgcacatgaacagcctgagagccgatgacacggccgtatattactgtgcgaaagatgaggcaccagctggcgcaaccttctttgactcctggggccagggaacgctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 353 - protein, 6 aa, Homo sapiens
QGIRRW

SEQ ID NO: 354 - skipped

SEQ ID NO: 355 - protein, 9 aa, Homo sapiens
QQANSFPIT

SEQ ID NO: 356 - protein, 11 aa, Homo sapiens
RASQGIRRWLA

SEQ ID NO: 357 - protein, 7 aa, Homo sapiens
GASSLQS

SEQ ID NO: 358 - protein, 9 aa, Homo sapiens
QQANSFPIT

SEQ ID NO: 359 - protein, 107 aa, Homo sapiens
DIQMTQSPSSVSASVGDRVTITCRASQGIRRWLAWYQQKPGKAPKLLISGASSLQSGVPSRFSGSGSGTDFTLIITSLQPEDFATYYCQQANSFPITFGQGTRLEIK

SEQ ID NO: 360 - DNA, 322 bp, Homo sapiens
  source 1..322: mol_type = unassigned DNA
gacatccagatgacccagtctccatcttccgtgtctgcatctgtaggagacagagtcaccatcacttgtcgggcgagtcagggtattaggaggtggttagcctggtatcagcagaaaccagggaaagcccctaaactcctgatctctggtgcatccagtttgcaaagtggggtcccatcaaggttcagcggcagtggatctgggacagatttcactctcatcattaccagtctgcagcctgaagattttgcaacttactattgtcaacaggctaacagtttcccgatcaccttcggccaagggacacgactggagatcaaac

SEQ ID NO: 361 - protein, 214 aa, Homo sapiens
DIQMTQSPSSVSASVGDRVTITCRASQGIRRWLAWYQQKPGKAPKLLISGASSLQSGVPSRFSGSGSGTDFTLIITSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 362 - DNA, 642 bp, Homo sapiens
  source 1..642: mol_type = unassigned DNA
gacatccagatgacccagtctccatcttccgtgtctgcatctgtaggagacagagtcaccatcacttgtcgggcgagtcagggtattaggaggtggttagcctggtatcagcagaaaccagggaaagcccctaaactcctgatctctggtgcatccagtttgcaaagtggggtcccatcaaggttcagcggcagtggatctgggacagatttcactctcatcattaccagtctgcagcctgaagattttgcaacttactattgtcaacaggctaacagtttcccgatcaccttcggccaagggacacgactggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 363 - protein, 8 aa, Homo sapiens
GYTFSTFG

SEQ ID NO: 364 - protein, 8 aa, Homo sapiens
ISAYNGDT

SEQ ID NO: 365 - protein, 14 aa, Homo sapiens
ARSSGHYYYYGMDV

SEQ ID NO: 366 - protein, 121 aa, Homo sapiens
QVQVVQSGAEVKKPGASVKVSCKASGYTFSTFGITWVRQAPGQGLEWMGWISAYNGDTNYAQNLQGRVIMTTDTSTSTAYMELRSLRSDDTAVYYCARSSGHYYYYGMDVWGQGTTVTVSS

SEQ ID NO: 367 - DNA, 363 bp, Homo sapiens
  source 1..363: mol_type = unassigned DNA
caggttcaggtggtgcagtctggagctgaggtgaagaagcctggggcctcagtgaaggtctcctgcaaggcttctggttacaccttttccacctttggtatcacctgggtgcgacaggcccctggacaagggcttgaatggatgggatggatcagcgcttacaatggtgacacaaactatgcacagaatctccagggcagagtcatcatgaccacagacacatccacgagcacagcctacatggagctgaggagcctgagatctgacgacacggccgtttattactgtgcgaggagcagtggccactactactactacggtatggacgtctggggccaagggaccacggtcaccgtctcctca

SEQ ID NO: 368 - protein, 451 aa, Homo sapiens
QVQVVQSGAEVKKPGASVKVSCKASGYTFSTFGITWVRQAPGQGLEWMGWISAYNGDTNYAQNLQGRVIMTTDTSTSTAYMELRSLRSDDTAVYYCARSSGHYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 369 - DNA, 1359 bp, Homo sapiens
  source 1..1359: mol_type = unassigned DNA
caggttcaggtggtgcagtctggagctgaggtgaagaagcctggggcctcagtgaaggtctcctgcaaggcttctggttacaccttttccacctttggtatcacctgggtgcgacaggcccctggacaagggcttgaatggatgggatggatcagcgcttacaatggtgacacaaactatgcacagaatctccagggcagagtcatcatgaccacagacacatccacgagcacagcctacatggagctgaggagcctgagatctgacgacacggccgtttattactgtgcgaggagcagtggccactactactactacggtatggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 370 - protein, 11 aa, Homo sapiens
QSLLHSNEYNY

SEQ ID NO: 371 - skipped

SEQ ID NO: 372 - protein, 9 aa, Homo sapiens
MQSLQTPLT

SEQ ID NO: 373 - protein, 112 aa, Homo sapiens
DIVMTQSPLSLPVTPGEPASISCRSSQSLLHSNEYNYLDWYLQKPGQSPQLLIFLGSNRASGVPDRFSGSGSGTDFTLKITRVEAEDVGIYYCMQSLQTPLTFGGGTKVEIK

SEQ ID NO: 374 - DNA, 336 bp, Homo sapiens
  source 1..336: mol_type = unassigned DNA
gatattgtgatgactcagtctccactctccctgcccgtcacccctggagagccggcctccatctcctgcaggtctagtcagagcctcctgcatagtaatgaatacaactatttggattggtacctgcagaagccagggcagtctccacagctcctgatctttttgggttctaatcgggcctccggggtccctgacaggttcagtggcagtggatcaggcacagattttacactgaaaatcaccagagtggaggctgaggatgttggaatttattactgcatgcaatctctacaaactccgctcactttcggcggagggaccaaggtggagatcaaa

SEQ ID NO: 375 - protein, 219 aa, Homo sapiens
DIVMTQSPLSLPVTPGEPASISCRSSQSLLHSNEYNYLDWYLQKPGQSPQLLIFLGSNRASGVPDRFSGSGSGTDFTLKITRVEAEDVGIYYCMQSLQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 376 - DNA, 657 bp, Homo sapiens
  source 1..657: mol_type = unassigned DNA
gatattgtgatgactcagtctccactctccctgcccgtcacccctggagagccggcctccatctcctgcaggtctagtcagagcctcctgcatagtaatgaatacaactatttggattggtacctgcagaagccagggcagtctccacagctcctgatctttttgggttctaatcgggcctccggggtccctgacaggttcagtggcagtggatcaggcacagattttacactgaaaatcaccagagtggaggctgaggatgttggaatttattactgcatgcaatctctacaaactccgctcactttcggcggagggaccaaggtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 377 - protein, 8 aa, Homo sapiens
GYTFTSYG

SEQ ID NO: 378 - protein, 8 aa, Homo sapiens
ISAYNGNT

SEQ ID NO: 379 - protein, 17 aa, Homo sapiens
ARSTYFYGSGTLYGMDV

SEQ ID NO: 380 - protein, 124 aa, Homo sapiens
QVQLVQSGGEVKKPGASVKVSCKASGYTFTSYGFSWVRQAPGQGLEWMGWISAYNGNTNYAQKLQGRVTMTTDTSTSTAYMELRSLRSDDTAVYYCARSTYFYGSGTLYGMDVWGQGTTVTVSS

SEQ ID NO: 381 - DNA, 372 bp, Homo sapiens
  source 1..372: mol_type = unassigned DNA
caggttcaactggtgcagtctggaggtgaggtgaagaagcctggggcctcagtgaaggtctcctgcaaggcttctggttacacctttaccagctatggtttcagctgggtgcgacaggcccctggacaaggactagagtggatgggatggatcagcgcttacaatggtaacacaaactatgcacagaagctccagggcagagtcaccatgaccacagacacatccacgagcacagcctacatggagctgaggagcttgagatctgacgacacggccgtgtattactgtgcgagatctacgtatttctatggttcggggaccctctacggtatggacgtctggggccaagggaccacggtcaccgtctcctca

SEQ ID NO: 382 - protein, 454 aa, Homo sapiens
QVQLVQSGGEVKKPGASVKVSCKASGYTFTSYGFSWVRQAPGQGLEWMGWISAYNGNTNYAQKLQGRVTMTTDTSTSTAYMELRSLRSDDTAVYYCARSTYFYGSGTLYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 383 - DNA, 1368 bp, Homo sapiens
  source 1..1368: mol_type = unassigned DNA
caggttcaactggtgcagtctggaggtgaggtgaagaagcctggggcctcagtgaaggtctcctgcaaggcttctggttacacctttaccagctatggtttcagctgggtgcgacaggcccctggacaaggactagagtggatgggatggatcagcgcttacaatggtaacacaaactatgcacagaagctccagggcagagtcaccatgaccacagacacatccacgagcacagcctacatggagctgaggagcttgagatctgacgacacggccgtgtattactgtgcgagatctacgtatttctatggttcggggaccctctacggtatggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 384 - protein, 11 aa, Homo sapiens
QSLLHSDGYNY

SEQ ID NO: 385 - skipped

SEQ ID NO: 386 - protein, 9 aa, Homo sapiens
MQALQTPLS

SEQ ID NO: 387 - protein, 112 aa, Homo sapiens
DIVMTQSPLSLPVTPGEPASISCRSSQSLLHSDGYNYLDWYLQKPGQSPQLLIYLGSTRASGFPDRFSGSGSGTDFTLKISRVEAEDVGVYYCMQALQTPLSFGQGTKLEIK

SEQ ID NO: 388 - DNA, 336 bp, Homo sapiens
  source 1..336: mol_type = unassigned DNA
gatattgtgatgactcagtctccactctccctgcccgtcacccctggagagccggcctccatctcctgcaggtctagtcagagcctcctgcatagtgatggatacaactgtttggattggtacctgcagaagccagggcagtctccacagctcctgatctatttgggttctactcgggcctccgggttccctgacaggttcagtggcagtggatcaggcacagattttacactgaaaatcagcagagtggaggctgaggatgttggggtttattactgcatgcaagctctacaaactccgtgcagttttggccaggggaccaagctggagatcaaa

SEQ ID NO: 389 - protein, 219 aa, Homo sapiens
DIVMTQSPLSLPVTPGEPASISCRSSQSLLHSDGYNYLDWYLQKPGQSPQLLIYLGSTRASGFPDRFSGSGSGTDFTLKISRVEAEDVGVYYCMQALQTPLSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 390 - DNA, 657 bp, Homo sapiens
  source 1..657: mol_type = unassigned DNA
gatattgtgatgactcagtctccactctccctgcccgtcacccctggagagccggcctccatctcctgcaggtctagtcagagcctcctgcatagtgatggatacaactgtttggattggtacctgcagaagccagggcagtctccacagctcctgatctatttgggttctactcgggcctccgggttccctgacaggttcagtggcagtggatcaggcacagattttacactgaaaatcagcagagtggaggctgaggatgttggggtttattactgcatgcaagctctacaaactccgtgcagttttggccaggggaccaagctggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 391 - protein, 8 aa, Homo sapiens
GYTFTSYG

SEQ ID NO: 392 - protein, 8 aa, Homo sapiens
ISAYNGNT

SEQ ID NO: 393 - protein, 17 aa, Homo sapiens
ARSTYFYGSGTLYGMDV

SEQ ID NO: 394 - protein, 124 aa, Homo sapiens; identical to SEQ ID NO: 380

SEQ ID NO: 395 - DNA, 372 bp, Homo sapiens; identical to SEQ ID NO: 381
  source 1..372: mol_type = unassigned DNA

SEQ ID NO: 396 - protein, 454 aa, Homo sapiens; identical to SEQ ID NO: 382

SEQ ID NO: 397 - DNA, 1368 bp, Homo sapiens; identical to SEQ ID NO: 383
  source 1..1368: mol_type = unassigned DNA

SEQ ID NO: 398 - protein, 11 aa, Homo sapiens
QSLLHSDGYNC

SEQ ID NO: 399 - skipped

SEQ ID NO: 400 - protein, 9 aa, Homo sapiens
MQALQTPCS

SEQ ID NO: 401 - protein, 112 aa, Homo sapiens
DIVMTQSPLSLPVTPGEPASISCRSSQSLLHSDGYNCLDWYLQKPGQSPQLLIYLGSTRASGFPDRFSGSGSGTDFTLKISRVEAEDVGVYYCMQALQTPCSFGQGTKLEIK

SEQ ID NO: 402 - DNA, 336 bp, Homo sapiens; identical to SEQ ID NO: 388
  source 1..336: mol_type = unassigned DNA

SEQ ID NO: 403 - protein, 219 aa, Homo sapiens
DIVMTQSPLSLPVTPGEPASISCRSSQSLLHSDGYNCLDWYLQKPGQSPQLLIYLGSTRASGFPDRFSGSGSGTDFTLKISRVEAEDVGVYYCMQALQTPCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 404 - DNA, 657 bp, Homo sapiens; identical to SEQ ID NO: 390
  source 1..657: mol_type = unassigned DNA

SEQ ID NO: 405 - protein, 8 aa, Homo sapiens
GVTFDDYG

SEQ ID NO: 406 - protein, 8 aa, Homo sapiens
INWNGGDT

SEQ ID NO: 407 - protein, 17 aa, Homo sapiens
ARDFYGSGSYYHVPFDY

SEQ ID NO: 408 - protein, 124 aa, Homo sapiens
EVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSS

SEQ ID NO: 409 - DNA, 372 bp, Homo sapiens
  source 1..372: mol_type = unassigned DNA
gaggtgcagctggtggagtctgggggaggtgtggtacggcctggggggtccctgagactctcctgtgtagcctctggagtcacctttgatgattatggcatgagctgggtccgccaagctccagggaaggggctggartgggtctctggtattaattggaatggtggcgacacagattattcagactctgtgaagggccgattcaccatctccagagacaacgccaagaactccctgtatctacaaatgaatagtctgagagccgaggacacggccttgtattactgtgcgagggatttctatggttcggggagttattatcacgttccttttgactactggggccagggaatcctggtcaccgtctcctca

SEQ ID NO: 410 - protein, 454 aa, Homo sapiens
EVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 411 - DNA, 1368 bp, Homo sapiens
  source 1..1368: mol_type = unassigned DNA
gaggtgcagctggtggagtctgggggaggtgtggtacggcctggggggtccctgagactctcctgtgtagcctctggagtcacctttgatgattatggcatgagctgggtccgccaagctccagggaaggggctggartgggtctctggtattaattggaatggtggcgacacagattattcagactctgtgaagggccgattcaccatctccagagacaacgccaagaactccctgtatctacaaatgaatagtctgagagccgaggacacggccttgtattactgtgcgagggatttctatggttcggggagttattatcacgttccttttgactactggggccagggaatcctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 412 - protein, 7 aa, Homo sapiens
QSVSRSY

SEQ ID NO: 413 - skipped

SEQ ID NO: 414 - protein, 9 aa, Homo sapiens
HQYDMSPFT

SEQ ID NO: 415 - protein, 108 aa, Homo sapiens
EIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIK

SEQ ID NO: 416 - DNA, 324 bp, Homo sapiens
  source 1..324: mol_type = unassigned DNA
gaaattgtgttgacgcagtctccagggaccctgtctttgtctccaggggaaagagccaccctctcctgcagggccagtcagagtgttagcagaagctacttagcctggtaccagcagaaacgtggccaggctcccaggctcctcatctatggtgcatccagcagggccactggcatcccagacaggttcagtggcgatgggtctgggacagacttcactctctccatcagcagactggagcctgaagattttgcagtgtattactgtcaccagtatgatatgtcaccattcactttcggccctgggaccaaagtggatatcaaa

SEQ ID NO: 417 - protein, 215 aa, Homo sapiens
EIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 418 - DNA, 645 bp, Homo sapiens
  source 1..645: mol_type = unassigned DNA
gaaattgtgttgacgcagtctccagggaccctgtctttgtctccaggggaaagagccaccctctcctgcagggccagtcagagtgttagcagaagctacttagcctggtaccagcagaaacgtggccaggctcccaggctcctcatctatggtgcatccagcagggccactggcatcccagacaggttcagtggcgatgggtctgggacagacttcactctctccatcagcagactggagcctgaagattttgcagtgtattactgtcaccagtatgatatgtcaccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 419 - protein, 8 aa, Homo sapiens
GLTFDDYG

SEQ ID NO: 420 - protein, 8 aa, Homo sapiens
INWNGDNT

SEQ ID NO: 421 - protein, 17 aa, Homo sapiens
ARDYYGSGSYYNVPFDY

SEQ ID NO: 422 - protein, 124 aa, Homo sapiens
EVQLVESGGGVVRPGGSLRLSCAASGLTFDDYGMSWVRQVPGKGLEWVSGINWNGDNTDYADSVKGRFTISRDNAKNSLYLQMNSLRAEDTALYYCARDYYGSGSYYNVPFDYWGQGTLVTVSS

SEQ ID NO: 423 - DNA, 372 bp, Homo sapiens
  source 1..372: mol_type = unassigned DNA
gaggtgcagctggtggagtctgggggaggtgtggtacggcctggggggtccctgagactctcctgtgcagcctctggactcacctttgatgattatggcatgagctgggtccgccaagttccagggaaggggctggagtgggtctctggtattaattggaatggtgataacacagattatgcagactctgtgaagggccgattcaccatctccagagacaacgccaagaactccctgtatctgcaaatgaacagtctgagagccgaggacacggccttgtattactgtgcgagggattactatggttcggggagttattataacgttccttttgactactggggccagggaaccctggtcaccgtctcctca

SEQ ID NO: 424 - protein, 454 aa, Homo sapiens
EVQLVESGGGVVRPGGSLRLSCAASGLTFDDYGMSWVRQVPGKGLEWVSGINWNGDNTDYADSVKGRFTISRDNAKNSLYLQMNSLRAEDTALYYCARDYYGSGSYYNVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 425 - DNA, 1368 bp, Homo sapiens
  source 1..1368: mol_type = unassigned DNA
gaggtgcagctggtggagtctgggggaggtgtggtacggcctggggggtccctgagactctcctgtgcagcctctggactcacctttgatgattatggcatgagctgggtccgccaagttccagggaaggggctggagtgggtctctggtattaattggaatggtgataacacagattatgcagactctgtgaagggccgattcaccatctccagagacaacgccaagaactccctgtatctgcaaatgaacagtctgagagccgaggacacggccttgtattactgtgcgagggattactatggttcggggagttattataacgttccttttgactactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 426 - protein, 7 aa, Homo sapiens
QSVSSSY

SEQ ID NO: 427 - skipped

SEQ ID NO: 428 - protein, 8 aa, Homo sapiens
QQYGSSPF

SEQ ID NO: 429 - protein, 107 aa, Homo sapiens
EIVLTQSPGTLSLSPGERATLSCRASQSVSSSYLAWYQQKPGQAPRLLIYGASSRATGIPDRFSGSGSGTDFTLTIRRLEPEDFAVYYCQQYGSSPFFGPGTKVDIK

SEQ ID NO: 430 - DNA, 321 bp, Homo sapiens
  source 1..321: mol_type = unassigned DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatatatggtgcatccagcagggccactggcatcccagacaggttcagtggcagtgggtctgggacagacttcactctcaccatcagaagactggagcctgaagattttgcagtgtattactgtcagcagtatggtagttcaccattcttcggccctgggaccaaagtggatatcaaa

SEQ ID NO: 431 - DNA, 323 bp, Homo sapiens
  source 1..323: mol_type = unassigned DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatatatggtgcatccagcagggccactggcatcccagacaggttcagtggcagtgggtctgggacagacttcactctcaccatcagaagactggagcctgaagattttgcagtgtattactgtcagcagtatggtagttcaccattcacttcggccctgggaccaaagtggatatcaaa

SEQ ID NO: 432 - protein, 214 aa, Homo sapiens
EIVLTQSPGTLSLSPGERATLSCRASQSVSSSYLAWYQQKPGQAPRLLIYGASSRATGIPDRFSGSGSGTDFTLTIRRLEPEDFAVYYCQQYGSSPFFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 433 - DNA, 642 bp, Homo sapiens
  source 1..642: mol_type = unassigned DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatatatggtgcatccagcagggccactggcatcccagacaggttcagtggcagtgggtctgggacagacttcactctcaccatcagaagactggagcctgaagattttgcagtgtattactgtcagcagtatggtagttcaccattcttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 434 - DNA, 644 bp, Homo sapiens
  source 1..644: mol_type = unassigned DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatatatggtgcatccagcagggccactggcatcccagacaggttcagtggcagtgggtctgggacagacttcactctcaccatcagaagactggagcctgaagattttgcagtgtattactgtcagcagtatggtagttcaccattcacttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 435 - protein, 8 aa, Homo sapiens
GYTFNSYG

SEQ ID NO: 436 - protein, 8 aa, Homo sapiens
ISVHNGNT

SEQ ID NO: 437 - protein, 17 aa, Homo sapiens
ARAGYDILTDFSDAFDI

SEQ ID NO: 438 - protein, 124 aa, Homo sapiens
QVQLVQSGAEVKKPGASVKVSCKASGYTFNSYGIIWVRQAPGQGLEWMGWISVHNGNTNCAQKLQGRVTMTTDTSTSTAYMELRSLRTDDTAVYYCARAGYDILTDFSDAFDIWGHGTMVTVSS

SEQ ID NO: 439 - DNA, 372 bp, Homo sapiens
  source 1..372: mol_type = unassigned DNA
caggttcagttggtgcagtctggagctgaggtgaagaagcctggggcctcagtgaaggtctcctgcaaggcttctggttacacctttaatagttatggtatcatctgggtgcgacaggcccctggacaagggcttgagtggatgggatggatcagcgttcacaatggtaacacaaactgtgcacagaagctccagggtagagtcaccatgaccacagacacatccacgagcacagcctacatggagctgaggagcctgagaactgacgacacggccgtgtattactgtgcgagagcgggttacgatattttgactgatttttccgatgcttttgatatctggggccacgggacaatggtcaccgtctcttca

SEQ ID NO: 440 - protein, 454 aa, Homo sapiens
QVQLVQSGAEVKKPGASVKVSCKASGYTFNSYGIIWVRQAPGQGLEWMGWISVHNGNTNCAQKLQGRVTMTTDTSTSTAYMELRSLRTDDTAVYYCARAGYDILTDFSDAFDIWGHGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 441 - DNA, 1368 bp, Homo sapiens
  source 1..1368: mol_type = unassigned DNA
caggttcagttggtgcagtctggagctgaggtgaagaagcctggggcctcagtgaaggtctcctgcaaggcttctggttacacctttaatagttatggtatcatctgggtgcgacaggcccctggacaagggcttgagtggatgggatggatcagcgttcacaatggtaacacaaactgtgcacagaagctccagggtagagtcaccatgaccacagacacatccacgagcacagcctacatggagctgaggagcctgagaactgacgacacggccgtgtattactgtgcgagagcgggttacgatattttgactgatttttccgatgcttttgatatctggggccacgggacaatggtcaccgtctcttcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 442 - protein, 6 aa, Homo sapiens
QNINNF

SEQ ID NO: 443 - skipped

SEQ ID NO: 444 - protein, 8 aa, Homo sapiens
QQSYGIPW

SEQ ID NO: 445 - protein, 106 aa, Homo sapiens
DIQMTQSPSSLSASVGDRVTITCRASQNINNFLNWYQQKEGKGPKLLIYAASSLQRGIPSTFSGSGSGTDFTLTISSLQPEDFATYICQQSYGIPWVGQGTKVEIK

SEQ ID NO: 446 - DNA, 318 bp, Homo sapiens
  source 1..318: mol_type = unassigned DNA
gacatccagatgacccagtctccatcctccctgtctgcatctgtaggagacagagtcaccatcacttgccgggcaagtcagaacattaataactttttaaattggtatcagcagaaagaagggaaaggccctaagctcctgatctatgcagcatccagtttgcaaagagggataccatcaacgttcagtggcagtggatctgggacagacttcactctcaccatcagcagtctgcaacctgaagattttgcaacttacatctgtcaacagagctacggtatcccgtgggtcggccaagggaccaaggtggaaatcaaa

SEQ ID NO: 447 - protein, 213 aa, Homo sapiens
DIQMTQSPSSLSASVGDRVTITCRASQNINNFLNWYQQKEGKGPKLLIYAASSLQRGIPSTFSGSGSGTDFTLTISSLQPEDFATYICQQSYGIPWVGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 448 - DNA, 639 bp, Homo sapiens
  source 1..639: mol_type = unassigned DNA
gacatccagatgacccagtctccatcctccctgtctgcatctgtaggagacagagtcaccatcacttgccgggcaagtcagaacattaataactttttaaattggtatcagcagaaagaagggaaaggccctaagctcctgatctatgcagcatccagtttgcaaagagggataccatcaacgttcagtggcagtggatctgggacagacttcactctcaccatcagcagtctgcaacctgaagattttgcaacttacatctgtcaacagagctacggtatcccgtgggtcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 449 - protein, 8 aa, Homo sapiens
GFTFSDYF

SEQ ID NO: 450 - protein, 8 aa, Homo sapiens
ISSSGSTI

SEQ ID NO: 451 - protein, 21 aa, Homo sapiens
ARDHYDGSGIYPLYYYYGLDV

SEQ ID NO: 452 - protein, 128 aa, Homo sapiens
QVQLVESGGGLVKPGGSLRLSCAASGFTFSDYFMSWIRQAPGKGLEWISYISSSGSTIYYADSVRGRFTISRDNAKYSLYLQMNSLRSEDTAVYYCARDHYDGSGIYPLYYYYGLDVWGQGTTVTVSS

SEQ ID NO: 453 - DNA, 384 bp, Homo sapiens
  source 1..384: mol_type = unassigned DNA
caggtgcagctggtggagtctgggggaggcttggtcaagcctggagggtccctgagactctcctgtgcagcctctggattcaccttcagtgactacttcatgagctggatccgccaggcgccagggaaggggctggagtggatttcatacattagttctagtggtagtaccatatactacgcagactctgtgaggggccgattcaccatctccagggacaacgccaagtactcactgtatctgcaaatgaacagcctgagatccgaggacacggccgtgtattactgtgcgagagatcactacgatggttcggggatttatcccctctactactattacggtttggacgtctggggccaggggaccacggtcaccgtctcctca

SEQ ID NO: 454 - protein, 458 aa, Homo sapiens
QVQLVESGGGLVKPGGSLRLSCAASGFTFSDYFMSWIRQAPGKGLEWISYISSSGSTIYYADSVRGRFTISRDNAKYSLYLQMNSLRSEDTAVYYCARDHYDGSGIYPLYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 455 - DNA, 1380 bp, Homo sapiens
  source 1..1380: mol_type = unassigned DNA
caggtgcagctggtggagtctgggggaggcttggtcaagcctggagggtccctgagactctcctgtgcagcctctggattcaccttcagtgactacttcatgagctggatccgccaggcgccagggaaggggctggagtggatttcatacattagttctagtggtagtaccatatactacgcagactctgtgaggggccgattcaccatctccagggacaacgccaagtactcactgtatctgcaaatgaacagcctgagatccgaggacacggccgtgtattactgtgcgagagatcactacgatggttcggggatttatcccctctactactattacggtttggacgtctggggccaggggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 456 - protein, 11 aa, Homo sapiens
QSLLHSNGYNY

SEQ ID NO: 457 - skipped

SEQ ID NO: 458 - protein, 9 aa, Homo sapiens
MQALQTPRS

SEQ ID NO: 459 - protein, 111 aa, Homo sapiens
IVMTQSPLSLPVTPGEPASISCRSSQSLLHSNGYNYLDYYLQKPGQSPQLLIYLGSYRASGVPDRFSGSGSGTDFTLKISRVEAEDVGVYYCMQALQTPRSFGQGTTLEIK

SEQ ID NO: 460 - DNA, 333 bp, Homo sapiens
  source 1..333: mol_type = unassigned DNA
attgtgatgactcagtctccactctccctacccgtcacccctggagagccggcctccatctcctgcaggtctagtcagagcctcctgcatagtaatggatacaactatttggattattacctgcagaagccagggcagtctccacagctcctgatctatttgggttcttatcgggcctccggggtccctgacaggttcagtggcagtggatcaggcacagattttacactgaaaatcagcagagtggaggctgaggatgttggggtttattactgcatgcaagctctacaaactcctcgcagttttggccaggggaccacgctggagatcaaa

SEQ ID NO: 461 - protein, 218 aa, Homo sapiens
IVMTQSPLSLPVTPGEPASISCRSSQSLLHSNGYNYLDYYLQKPGQSPQLLIYLGSYRASGVPDRFSGSGSGTDFTLKISRVEAEDVGVYYCMQALQTPRSFGQGTTLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 462 - DNA, 654 bp, Homo sapiens
  source 1..654: mol_type = unassigned DNA
attgtgatgactcagtctccactctccctacccgtcacccctggagagccggcctccatctcctgcaggtctagtcagagcctcctgcatagtaatggatacaactatttggattattacctgcagaagccagggcagtctccacagctcctgatctatttgggttcttatcgggcctccggggtccctgacaggttcagtggcagtggatcaggcacagattttacactgaaaatcagcagagtggaggctgaggatgttggggtttattactgcatgcaagctctacaaactcctcgcagttttggccaggggaccacgctggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 463 - protein, 10 aa, Homo sapiens
GFSLSTTGVG

SEQ ID NO: 464 - protein, 7 aa, Homo sapiens
IYWDDDK

SEQ ID NO: 465 - protein, 16 aa, Homo sapiens
THGYGSASYYHYGMDV

SEQ ID NO: 466 - protein, 124 aa, Homo sapiens
QITLKESGPTLVKPTQTLTLTCTFSGFSLSTTGVGVGWIRQPPGKALEWLAVIYWDDDKRYSPSLKSRLTITKDTSKNQVVLTMTNMDPVDTATYFCTHGYGSASYYHYGMDVWGQGTTVTVSS

SEQ ID NO: 467 - DNA, 372 bp, Homo sapiens
  source 1..372: mol_type = unassigned DNA
cagatcaccttgaaggagtctggtcctacgctggtgaaacccacacagaccctcacgctgacctgcaccttctctgggttctcactcagcactactggagtgggtgtgggctggatccgtcagcccccaggaaaggccctggagtggcttgcagtcatttattgggatgatgataagcgctacagcccatctctgaagagcagactcaccatcaccaaggacacctccaaaaaccaggtggtccttacaatgaccaacatggaccctgtggacacagccacatatttctgtacacacggatatggttcggcgagttattaccactacggtatggacgtctggggccaagggaccacggtcaccgtctcctca

SEQ ID NO: 468 - protein, 454 aa, Homo sapiens
QITLKESGPTLVKPTQTLTLTCTFSGFSLSTTGVGVGWIRQPPGKALEWLAVIYWDDDKRYSPSLKSRLTITKDTSKNQVVLTMTNMDPVDTATYFCTHGYGSASYYHYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 469 - DNA, 1368 bp, Homo sapiens
  source 1..1368: mol_type = unassigned DNA
cagatcaccttgaaggagtctggtcctacgctggtgaaacccacacagaccctcacgctgacctgcaccttctctgggttctcactcagcactactggagtgggtgtgggctggatccgtcagcccccaggaaaggccctggagtggcttgcagtcatttattgggatgatgataagcgctacagcccatctctgaagagcagactcaccatcaccaaggacacctccaaaaaccaggtggtccttacaatgaccaacatggaccctgtggacacagccacatatttctgtacacacggatatggttcggcgagttattaccactacggtatggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 470 - protein, 6 aa, Homo sapiens
QSVTNY

SEQ ID NO: 471 - skipped

SEQ ID NO: 472 - protein, 9 aa, Homo sapiens
QHRSNWPLT

SEQ ID NO: 473 - protein, 107 aa, Homo sapiens
EIVLTQSPATLSLSPGERATLSCRASQSVTNYLAWHQQKPGQAPRLLIYDASNRATGIPARFSGSGSGTDFTLTISSLEPEDFAVYYCQHRSNWPLTFGGGTKVEIK

SEQ ID NO: 474 - DNA, 322 bp, Homo sapiens
  source 1..322: mol_type = unassigned DNA
gaaattgtattgacacagtctccagccaccctgtctttgtctccaggggaaagagccaccctctcctgcagggccagtcagagtgttaccaactacttagcctggcaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcaccgtagcaactggcctctcactttcggcggagggaccaaggtggagatcaaac

SEQ ID NO: 475 - protein, 214 aa, Homo sapiens
EIVLTQSPATLSLSPGERATLSCRASQSVTNYLAWHQQKPGQAPRLLIYDASNRATGIPARFSGSGSGTDFTLTISSLEPEDFAVYYCQHRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 476 - DNA, 643 bp, Homo sapiens
  source 1..643: mol_type = unassigned DNA
gaaattgtattgacacagtctccagccaccctgtctttgtctccaggggaaagagccaccctctcctgcagggccagtcagagtgttaccaactacttagcctggcaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcaccgtagcaactggcctctcactttcggcggagggaccaaggtggagatcaaaccgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 477 - protein, 10 aa, Homo sapiens
GFSLSTSGVG

SEQ ID NO: 478 - protein, 7 aa, Homo sapiens
IYWDDDK

SEQ ID NO: 479 - protein, 16 aa, Homo sapiens
THGYGSASYYHYGMDV

SEQ ID NO: 480 - protein, 124 aa, Homo sapiens
QITLKESGPTLVKPTQTLTLTCTFSGFSLSTSGVGVGWIRQPPGKALEWLAVIYWDDDKRYSPSLKSRLTITKDTSKNQVVLTMTNMDPVDTATYFCTHGYGSASYYHYGMDVWGQGTTVTVSS

SEQ ID NO: 481 - DNA, 372 bp, Homo sapiens
  source 1..372: mol_type = unassigned DNA
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcagtcatttattgggatgatgataagcgctacagcccatctctgaagagcaggctcaccatcaccaaggacacctccaaaaaccaggtggtccttacaatgaccaacatggaccctgtggacacagccacatatttctgtacacacggatatggttcggcgagttattaccactacggtatggacgtctggggccaagggaccacggtcaccgtctcctca

SEQ ID NO: 482 - protein, 454 aa, Homo sapiens
QITLKESGPTLVKPTQTLTLTCTFSGFSLSTSGVGVGWIRQPPGKALEWLAVIYWDDDKRYSPSLKSRLTITKDTSKNQVVLTMTNMDPVDTATYFCTHGYGSASYYHYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 483 - DNA, 1368 bp, Homo sapiens
  source 1..1368: mol_type = unassigned DNA
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcagtcatttattgggatgatgataagcgctacagcccatctctgaagagcaggctcaccatcaccaaggacacctccaaaaaccaggtggtccttacaatgaccaacatggaccctgtggacacagccacatatttctgtacacacggatatggttcggcgagttattaccactacggtatggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 484 - protein, 6 aa, Homo sapiens
QSVTNY

SEQ ID NO: 485 - skipped

SEQ ID NO: 486 - protein, 9 aa, Homo sapiens
QQRSNWPLT

SEQ ID NO: 487 - protein, 107 aa, Homo sapiens
EIVLTQSPATLSLSPGERATLSCRASQSVTNYLAWHQQKPGQAPRLLIYDASNRATGIPARFSGSGSGTDFTLTISSLEPEDFAVYYCQQRSNWPLTFGGGTKVEIK

SEQ ID NO: 488 - DNA, 321 bp, Homo sapiens
  source 1..321: mol_type = unassigned DNA
gaaattgtgttgacacagtctccagccaccctgtctttgtctccaggggaaagagccaccctctcctgcagggccagtcagagtgttaccaactacttagcctggcaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaa

SEQ ID NO: 489 - protein, 214 aa, Homo sapiens
EIVLTQSPATLSLSPGERATLSCRASQSVTNYLAWHQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 490 - DNA, 642 bp, Homo sapiens
  source 1..642: mol_type = unassigned DNA
gaaattgtgttgacacagtctccagccaccctgtctttgtctccaggggaaagagccaccctctcctgcagggccagtcagagtgttaccaactacttagcctggcaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 491 - protein, 8 aa, Homo sapiens
GFTFSDYY

SEQ ID NO: 492 - protein, 8 aa, Homo sapiens
ISSSGSTI

SEQ ID NO: 493 - protein, 20 aa, Homo sapiens
ARDFYDILTDSPYFYYGVDV

SEQ ID NO: 494 - protein, 127 aa, Homo sapiens
QVQLVESGGGLVKPGGSLRLSCAASGFTFSDYYMSWIRQAPGKGLEWVSYISSSGSTIYYADSVKGRFTISRDNAKNSLYLQINSLRAEDTAVYYCARDFYDILTDSPYFYYGVDVWGQGTTVTVSS

SEQ ID NO: 495 - DNA, 381 bp, Homo sapiens
  source 1..381: mol_type = unassigned DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaattaacagcctgagagccgaggacacggccgtgtattactgtgcgagagatttttacgatattttgactgatagtccgtacttctactacggtgtggacgtctggggccaagggaccacggtcaccgtctcctca

SEQ ID NO: 496 - protein, 457 aa, Homo sapiens
QVQLVESGGGLVKPGGSLRLSCAASGFTFSDYYMSWIRQAPGKGLEWVSYISSSGSTIYYADSVKGRFTISRDNAKNSLYLQINSLRAEDTAVYYCARDFYDILTDSPYFYYGVDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 497 - DNA, 1377 bp, Homo sapiens
  source 1..1377: mol_type = unassigned DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaattaacagcctgagagccgaggacacggccgtgtattactgtgcgagagatttttacgatattttgactgatagtccgtacttctactacggtgtggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 498 - protein, 11 aa, Homo sapiens
QSLLHSNGYNY

SEQ ID NO: 499 - skipped

SEQ ID NO: 500 - protein, 9 aa, Homo sapiens
MQALQTPRT

SEQ ID NO: 501 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGQGTKVEIK

SEQ ID NO: 502 - DNA, 336 bp, Homo sapiens
  source 1..336: mol_type = unassigned DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcggacgttcggccaagggaccaaggtggaaatcaaa

SEQ ID NO: 503 - protein, 219 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 504 - DNA, 657 bp, Homo sapiens
  source 1..657: mol_type = unassigned DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 505 - protein, 239 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTIEGRDYKDDDDKHHHHHH

SEQ ID NO: 506 - protein, 179 aa, Homo sapiens
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 507 - protein, 121 aa, Homo sapiens
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKF

SEQ ID NO: 508 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 509 - protein, 33 aa, Homo sapiens
KYSSSVHDPNGEYMFMRAVNTAKKSRLTDVTLM

SEQ ID NO: 510 - protein, 128 aa, Homo sapiens
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKIVVQVTE

SEQ ID NO: 511 - protein, 121 aa, Homo sapiens
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLK

SEQ ID NO: 512 - protein, 147 aa, Homo sapiens
MGWSCIILFLVATATGVHSEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKIVVQVTE

SEQ ID NO: 513 - protein, 199 aa, Macaca fascicularis
MKSGLWYFFLFCLHMKVLTGEINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKFWLPIGCATFVVVCIFGCILICWLTKKKYSSTVHDPNGEYMFMRAVNTAKKSRLTGTTP

SEQ ID NO: 514 - protein, 120 aa, Macaca fascicularis
EINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLK

SEQ ID NO: 515 - protein, 240 aa, Homo sapiens
DTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWS

SEQ ID NO: 516 - protein, 302 aa, Homo sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 517 - protein, 113 aa, Homo sapiens
LQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 518 - protein, 113 aa, Homo sapiens
LQMILNGINNYKNPKLTQMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 519 - DNA, 336 bp, Homo sapiens
  source 1..336: mol_type = unassigned DNA
gatattgtgatgactcagtctccactctccctgcccgtcacccctggagagccggcctccatctcctgcaggtctagtcagagcctcctgcatagtgatggatacaactatttggattggtacctgcagaagccagggcagtctccacagctcctgatctatttgggttctactcgggcctccgggttccctgacaggttcagtggcagtggatcaggcacagattttacactgaaaatcagcagagtggaggctgaggatgttggggtttattactgcatgcaagctctacaaactccgctcagttttggccaggggaccaagctggagatcaaa

SEQ ID NO: 520 - DNA, 657 bp, Homo sapiens
  source 1..657: mol_type = unassigned DNA
gatattgtgatgactcagtctccactctccctgcccgtcacccctggagagccggcctccatctcctgcaggtctagtcagagcctcctgcatagtgatggatacaactatttggattggtacctgcagaagccagggcagtctccacagctcctgatctatttgggttctactcgggcctccgggttccctgacaggttcagtggcagtggatcaggcacagattttacactgaaaatcagcagagtggaggctgaggatgttggggtttattactgcatgcaagctctacaaactccgctcagttttggccaggggaccaagctggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 521 - DNA, 372 bp, Homo sapiens
  source 1..372: mol_type = unassigned DNA
gaggtgcagctggtggagtctgggggaggtgtggtacggcctggggggtccctgagactctcctgtgtagcctctggagtcacctttgatgattatggcatgagctgggtccgccaagctccagggaaggggctggagtgggtctctggtattaattggaatggtggcgacacagattattcagactctgtgaagggccgattcaccatctccagagacaacgccaagaactccctgtatctacaaatgaatagtctgagagccgaggacacggccttgtattactgtgcgagggatttctatggttcggggagttattatcacgttccttttgactactggggccagggaatcctggtcaccgtctcctca

SEQ ID NO: 522 - DNA, 1368 bp, Homo sapiens
  source 1..1368: mol_type = unassigned DNA
gaggtgcagctggtggagtctgggggaggtgtggtacggcctggggggtccctgagactctcctgtgtagcctctggagtcacctttgatgattatggcatgagctgggtccgccaagctccagggaaggggctggagtgggtctctggtattaattggaatggtggcgacacagattattcagactctgtgaagggccgattcaccatctccagagacaacgccaagaactccctgtatctacaaatgaatagtctgagagccgaggacacggccttgtattactgtgcgagggatttctatggttcggggagttattatcacgttccttttgactactggggccagggaatcctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 523 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 524 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 525 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacatcttctcatgctccgtgatgcatgaggctctgcacaaccactacacgcagaagagcctctccctgtctccgggtaaa

SEQ ID NO: 526 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 527 - DNA, 978 bp, Homo sapiens
  source 1..978: mol_type = unassigned DNA
gcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 528 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 529 - DNA, 978 bp, Homo sapiens
  source 1..978: mol_type = unassigned DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgacctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 530 - protein, 326 aa, Homo sapiens
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 531 - DNA, 978 bp, Homo sapiens
  source 1..978: mol_type = unassigned DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcagcttgggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 532 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 533 - DNA, 978 bp, Homo sapiens
  source 1..978: mol_type = unassigned DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 534 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 535 - DNA, 318 bp, Homo sapiens
  source 1..318: mol_type = unassigned DNA
ggtcagcccaaggctgccccctcggtcactctgttcccaccctcctctgaggagcttcaagccaacaaggccacactggtgtgtctcgtaagtgacttcaacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 536 - protein, 106 aa, Homo sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 537 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 538 - protein, 106 aa, Homo sapiens; identical to SEQ ID NO: 225

SEQ ID NO: 539 - protein, 663 aa, Homo sapiens
DIQMTQSPASLSASLGETVTIQCRASEDIYSGLAWFQQKPGKSPQLLIYGASSLQDGVPSRFSGSGSGTQYSLKISSMQTEDEGVYFCQQGLKYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 540 - protein, 215 aa, Homo sapiens
EVQLVESGGGLVQPGRSLKLSCAASGFTFSDFYMAWVRQAPKKGLEWVASISYEGSSTYYGDSVMGRFTISRDNAKSTLYLQMNSLRSEDTATYYCARQREANWEDWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 541 - protein, 220 aa, Homo sapiens
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 542 - protein, 667 aa, Homo sapiens
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGRSLKLSCAASGFTFSDFYMAWVRQAPKKGLEWVASISYEGSSTYYGDSVMGRFTISRDNAKSTLYLQMNSLRSEDTATYYCARQREANWEDWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 543 - protein, 217 aa, Homo sapiens
EVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 544 - protein, 214 aa, Homo sapiens
DIQMTQSPASLSASLGETVTIQCRASEDIYSGLAWFQQKPGKSPQLLIYGASSLQDGVPSRFSGSGSGTQYSLKISSMQTEDEGVYFCQQGLKYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 545 - protein, 663 aa, Homo sapiens
DIQMTQSPASLSASLGETVTIQCRASEDIYSGLAWFQQKPGKSPQLLIYGASSLQDGVPSRFSGSGSGTQYSLKISSMQTEDEGVYFCQQGLKYPPTFGSGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECEVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 546 - protein, 214 aa, Homo sapiens
EVQLVESGGGLVQPGRSLKLSCAASGFTFSDFYMAWVRQAPKKGLEWVASISYEGSSTYYGDSVMGRFTISRDNAKSTLYLQMNSLRSEDTATYYCARQREANWEDWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKI

SEQ ID NO: 547 - protein, 220 aa, Homo sapiens
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 548 - protein, 667 aa, Homo sapiens
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECEVQLVESGGGLVQPGRSLKLSCAASGFTFSDFYMAWVRQAPKKGLEWVASISYEGSSTYYGDSVMGRFTISRDNAKSTLYLQMNSLRSEDTATYYCARQREANWEDWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 549 - protein, 216 aa, Homo sapiens
EVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKI

SEQ ID NO: 550 - protein, 214 aa, Homo sapiens
DIQMTQSPASLSASLGETVTIQCRASEDIYSGLAWFQQKPGKSPQLLIYGASSLQDGVPSRFSGSGSGTQYSLKISSMQTEDEGVYFCQQGLKYPPTFGSGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 551 - protein, 659 aa, Homo sapiens
DIQMTQSPASLSASLGETVTIQCRASEDIYSGLAWFQQKPGKSPQLLIYGASSLQDGVPSRFSGSGSGTQYSLKISSMQTEDEGVYFCQQGLKYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 552 - protein, 215 aa, Homo sapiens; identical to SEQ ID NO: 540

SEQ ID NO: 553 - protein, 220 aa, Homo sapiens; identical to SEQ ID NO: 541

SEQ ID NO: 554 - protein, 663 aa, Homo sapiens
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGRSLKLSCAASGFTFSDFYMAWVRQAPKKGLEWVASISYEGSSTYYGDSVMGRFTISRDNAKSTLYLQMNSLRSEDTATYYCARQREANWEDWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 555 - protein, 217 aa, Homo sapiens; identical to SEQ ID NO: 543

SEQ ID NO: 556 - protein, 214 aa, Homo sapiens; identical to SEQ ID NO: 544

SEQ ID NO: 557 - protein, 659 aa, Homo sapiens; identical to SEQ ID NO: 551

SEQ ID NO: 558 - protein, 215 aa, Homo sapiens; identical to SEQ ID NO: 540

SEQ ID NO: 559 - protein, 220 aa, Homo sapiens; identical to SEQ ID NO: 541

SEQ ID NO: 560 - protein, 663 aa, Homo sapiens; identical to SEQ ID NO: 554

SEQ ID NO: 561 - protein, 217 aa, Homo sapiens; identical to SEQ ID NO: 543

SEQ ID NO: 562 - protein, 214 aa, Homo sapiens; identical to SEQ ID NO: 544

SEQ ID NO: 563 - DNA, 364 bp, Homo sapiens
  source 1..364: mol_type = unassigned DNA
caggttcaactgatgcagtctggaactgaggtgaagaagcctggggcctcagtgaaggtctcctgcaagacttctggttacacctttaccacctatggtatcacttgggtgcgacaggcccctggacaagggcttgagtggatgggatggatcagcgcttacagtggtgacacagactatgcacagaagttccagggcagagtcaccgtgacaacagacacatccacgaacacagcctacatggagttgaggagcctgaaatctgacgacacggccgtgtattattgtgcgagaagtagtggctggccccaccactacggtatggacgtctggggccaagggaccacggtcaccgtctcctcag

SEQ ID NO: 564 - protein, 121 aa, Homo sapiens
QVQLMQSGTEVKKPGASVKVSCKTSGYTFTTYGITWVRQAPGQGLEWMGWISAYSGDTDYAQKFQGRVTVTTDTSTNTAYMELRSLKSDDTAVYYCARSSGWPHHYGMDVWGQGTTVTVSS

SEQ ID NO: 565 - protein, 8 aa, Homo sapiens
GYTFTTYG

SEQ ID NO: 566 - protein, 8 aa, Homo sapiens
ISAYSGDT

SEQ ID NO: 567 - protein, 14 aa, Homo sapiens
ARSSGWPHHYGMDV

SEQ ID NO: 568 - DNA, 373 bp, Homo sapiens
  source 1..373: mol_type = unassigned DNA
caggttcaactggtgcagtctggaggtgaggtgaaaaagcctggggcctcagtgaaggtctcctgcaaggcttctggttacacctttaccagctatggtttcagctgggtgcgacaggcccctggacaaggactagagtggatgggatggatcagcgcttacaatggtaacacaaactatgcacagaagctccagggcagagtctccatgaccacagacacatccacgagcacagcctacatggagctgaggagcttgagatctgacgacacggccgtgtatttctgtgcgcgatctacgtcttactatggttcggggaccctatacggtatggacgtctggggccaagggaccacggtcaccgtctcctcag

SEQ ID NO: 569 - protein, 124 aa, Homo sapiens
QVQLVQSGGEVKKPGASVKVSCKASGYTFTSYGFSWVRQAPGQGLEWMGWISAYNGNTNYAQKLQGRVSMTTDTSTSTAYMELRSLRSDDTAVYFCARSTSYYGSGTLYGMDVWGQGTTVTVSS

SEQ ID NO: 570 - protein, 17 aa, Homo sapiens
ARSTSYYGSGTLYGMDV

SEQ ID NO: 571 - DNA, 373 bp, Homo sapiens
  source 1..373: mol_type = unassigned DNA
caggttcaactggtgcagtctggaggtgaggtgaagaagcctggggcctcagtgaaggtctcctgcaaggcttctggttacacctttaccagctatggtttcagctgggtgcgacaggcccctggacaaggactagagtggatgggatggatcagcgcttacaatggtaacacaaactatgcacagaagctccagggcagagtctccatgaccacagacacatccacgagcacagcctacatggagctgaggagcttgagatctgacgacacggccgtgtattactgtgcgcgatctacgtcttactatggttcggggaccctctacggtatggacgtctggggccaagggaccacggtcaccgtctcctcag

SEQ ID NO: 572 - protein, 124 aa, Homo sapiens
QVQLVQSGGEVKKPGASVKVSCKASGYTFTSYGFSWVRQAPGQGLEWMGWISAYNGNTNYAQKLQGRVSMTTDTSTSTAYMELRSLRSDDTAVYYCARSTSYYGSGTLYGMDVWGQGTTVTVSS

SEQ ID NO: 573 - DNA, 373 bp, Homo sapiens; identical to SEQ ID NO: 568
  source 1..373: mol_type = unassigned DNA

SEQ ID NO: 574 - protein, 124 aa, Homo sapiens; identical to SEQ ID NO: 569

SEQ ID NO: 575 - DNA, 373 bp, Homo sapiens
  source 1..373: mol_type = unassigned DNA
caggttcaactggtgcagtctggaggtgaggtgaaaaagcctcgggcctcagtgaaggtctcctgcaaggcttctggttacacctttaccagctatgtgttcagctgggtgcgacatgccgctggacaaggactagagtggatgggatggatcagcggttacaatggtaacacaaactatgcacagaagctccagtgcggagtctcgatgaccgcagacacatccacgagcacagcctacatggagctgaggagcttgagatctgacgacacggccgtgtatttctgtgcgcgatctacgtcttactatggtgcggggaccctatacggtatggacgtctggggccaagggaccacggtcaccgtctcctcag

SEQ ID NO: 576 - protein, 124 aa, Homo sapiens
QVQLVQSGGEVKKPRASVKVSCKASGYTFTSYVFSWVRHAAGQGLEWMGWISGYNGNTNYAQKLQCGVSMTADTSTSTAYMELRSLRSDDTAVYFCARSTSYYGAGTLYGMDVWGQGTTVTVSS

SEQ ID NO: 577 - protein, 8 aa, Homo sapiens
GYTFTSYV

SEQ ID NO: 578 - protein, 8 aa, Homo sapiens
ISGYNGNT

SEQ ID NO: 579 - protein, 17 aa, Homo sapiens
ARSTSYYGAGTLYGMDV

SEQ ID NO: 580 - DNA, 373 bp, Homo sapiens
  source 1..373: mol_type = unassigned DNA
gaggtgcagctggtggagtctgggggaggtgtggtacggcctggggggtccctgagactctcctgtgcagcctctggattcacctttgatgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccttgtattactgtgcggccgattactatggttcggggagttattataacgtcccctttgactactggggccagggaaccctggtcaccgtctcctcag

SEQ ID NO: 581 - protein, 124 aa, Homo sapiens
EVQLVESGGGVVRPGGSLRLSCAASGFTFDDYGMSWVRQAPGKGLEWVSGINWNGGSTGYADSVKGRFTISRDNAKNSLYLQMNSLRAEDTALYYCAADYYGSGSYYNVPFDYWGQGTLVTVSS

SEQ ID NO: 582 - protein, 8 aa, Homo sapiens
GFTFDDYG

SEQ ID NO: 583 - protein, 8 aa, Homo sapiens
INWNGGST

SEQ ID NO: 584 - protein, 17 aa, Homo sapiens
AADYYGSGSYYNVPFDY

SEQ ID NO: 585 - DNA, 373 bp, Homo sapiens
  source 1..373: mol_type = unassigned DNA
gaggtgcagctggtggagtctgggggaggtgtgatacggcctggggggtccctgagactctcctgtgcagcctctggattcacctttgatgattatggcatgagctgggtccgccaagctccagggaaggggctggagtgggtctctggtattaattggattggtgataacacagattatgcagactctgtgaagggccgattcaccatctccagagacaacgccaagaactccctatatctgcaaatgaacagtctgagagccgaggacacggccttgtattactgtgcgagagattactttggttcggggagttattataacgttccctttgactactggggccagggaaccctggtcaccgtctcctcag

SEQ ID NO: 586 - protein, 124 aa, Homo sapiens
EVQLVESGGGVIRPGGSLRLSCAASGFTFDDYGMSWVRQAPGKGLEWVSGINWIGDNTDYADSVKGRFTISRDNAKNSLYLQMNSLRAEDTALYYCARDYFGSGSYYNVPFDYWGQGTLVTVSS

SEQ ID NO: 587 - protein, 8 aa, Homo sapiens
INWIGDNT

SEQ ID NO: 588 - protein, 17 aa, Homo sapiens
ARDYFGSGSYYNVPFDY

SEQ ID NO: 589 - DNA, 337 bp, Homo sapiens
  source 1..337: mol_type = unassigned DNA
gatattgtgatgactcagtctccactctccctgcccgtcacccctggagagccggcctccatctcctgcaggtctagtcagagcctcctgcatagtaatggattcaactatttcgattggtacctgcagaagccaggacagtctccacagctcctgatctttttggtttctaatcgggcctccggggtccctgacaggttcagtggcagtggatcaggcacagattttacactgaaaatcagcagagtggaggctgaggatgttgggatttattactgcatgcaagctctacaaactccgctcactttcggcggagggaccaaggtggagatcaaac

SEQ ID NO: 590 - protein, 112 aa, Homo sapiens
DIVMTQSPLSLPVTPGEPASISCRSSQSLLHSNGFNYFDWYLQKPGQSPQLLIFLVSNRASGVPDRFSGSGSGTDFTLKISRVEAEDVGIYYCMQALQTPLTFGGGTKVEIK

SEQ ID NO: 591 - protein, 11 aa, Homo sapiens
QSLLHSNGFNY

SEQ ID NO: 592 - skipped

SEQ ID NO: 593 - protein, 9 aa, Homo sapiens
MQALQTPLT

SEQ ID NO: 594 - DNA, 337 bp, Homo sapiens
  source 1..337: mol_type = unassigned DNA
gatattgtgatgactcagtctccactctccctgcccgtcacccctggagagccggcctccatctcctgcaggtctagtcagagcctcctgcatagtaatggatacaactgtttggattggtacctgcagaagccagggcagtctccacagctcctgatctatttgggttctactcgggcctccgggttccctgacaggttcagtggcagtggatcaggcacagattttacactgaaaatcagcagagtggaggctgaggatgttggggtttattactgcatgcaagctctacaaactccgtgcagttttggccaggggaccaagctggagatcaaac

SEQ ID NO: 595 - protein, 112 aa, Homo sapiens
DIVMTQSPLSLPVTPGEPASISCRSSQSLLHSNGYNCLDWYLQKPGQSPQLLIYLGSTRASGFPDRFSGSGSGTDFTLKISRVEAEDVGVYYCMQALQTPCSFGQGTKLEIK

SEQ ID NO: 596 - protein, 11 aa, Homo sapiens
QSLLHSNGYNC

SEQ ID NO: 597 - DNA, 337 bp, Homo sapiens; identical to SEQ ID NO: 594
  source 1..337: mol_type = unassigned DNA

SEQ ID NO: 598 - protein, 112 aa, Homo sapiens; identical to SEQ ID NO: 595

SEQ ID NO: 599 - DNA, 337 bp, Homo sapiens
  source 1..337: mol_type = unassigned DNA
gatattgtgatgactcagtctccactctccctgcccgtcacccctggagagccggcctccatctcctgcaggtctagtcagagcctcctgcatagtaatggatacaactgtttggattggtacctgcagaagccagggcagtctccacagctcctgatctatttgggttctactcgggcctccgggttccctgacaggttcagtggcagtggatcaggcacagattctacactgaaaatcagcagagtggaggctgaggatgttggggtttattactgcatgcaagctctacaaactccgtgcagttttggccaggggaccaagctggagatcaaac

SEQ ID NO: 600 - protein, 112 aa, Homo sapiens
DIVMTQSPLSLPVTPGEPASISCRSSQSLLHSNGYNCLDWYLQKPGQSPQLLIYLGSTRASGFPDRFSGSGSGTDSTLKISRVEAEDVGVYYCMQALQTPCSFGQGTKLEIK

SEQ ID NO: 601 - DNA, 337 bp, Homo sapiens; identical to SEQ ID NO: 594
  source 1..337: mol_type = unassigned DNA

SEQ ID NO: 602 - protein, 112 aa, Homo sapiens; identical to SEQ ID NO: 595

SEQ ID NO: 603 - DNA, 325 bp, Homo sapiens
  source 1..325: mol_type = unassigned DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctttcactttcggccctgggaccaaagtggatatcaaac

SEQ ID NO: 604 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVDIK

SEQ ID NO: 605 - protein, 9 aa, Homo sapiens
QQYGSSPFT

SEQ ID NO: 606 - DNA, 325 bp, Homo sapiens
  source 1..325: mol_type = unassigned DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtatattactgtcaccagtatggtaattcaccattcactttcggccctgggaccaaagtggatatcaaac

SEQ ID NO: 607 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCHQYGNSPFTFGPGTKVDIK

SEQ ID NO: 608 - protein, 9 aa, Homo sapiens
HQYGNSPFT

SEQ ID NO: 609 - protein, 11 aa, Homo sapiens
  VAR_SEQ 1: note = X = any amino acid
  VAR_SEQ 5: note = X = any amino acid
  VAR_SEQ 8: note = X = any amino acid
  VAR_SEQ 9: note = X = is either present or absent, and if present, may be any amino acid
XGSGXYGXXFD

SEQ ID NO: 610 - protein, 478 aa, synthetic construct
  REGION 1..478: note = ICOSL-Fc fusion protein
DTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSDIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0190964 A1
(43) Pub. Date: Jun. 13, 2024

(54) TREATMENT OF PD-L1 NEGATIVE OR LOW EXPRESSING CANCER WITH ANTI-ICOS ANTIBODIES

(71) Applicant: Kymab Limited, Cambridge (GB)

(72) Inventors: Anthea NEWTON, Cambridge (GB); Cintia Cristina PALU, Cambridge (GB); Sonia QUARATINO, Cambridge (GB); Richard Charles Alfred SAINSON, Cambridge (GB); Cecilia DEANTONIO, Cambridge (GB); Matthew Stephen WAKE, Cambridge (GB)

(21) Appl. No.: 18/510,228

(22) Filed: Nov. 15, 2023

Related U.S. Application Data
(63) Continuation of application No. PCT/GB2022/051413, filed on Jun. 6, 2022.

(30) Foreign Application Priority Data
Jun. 4, 2021 (GB) 2107994.2

(51) Int. Cl. C07K 16/28; A61P 35/00

(52) U.S. Cl. CPC C07K 16/2818; A61P 35/00; C07K 16/2827; C07K 2317/31

(57) ABSTRACT

This invention relates to compositions and methods for the treatment of cancer, in particular difficult-to-treat cancers. More specifically, the present invention relates to compositions and methods for the treatment of PD-L1 negative or PD-L1 low expressing cancers using a modulator of ICOS, such as an anti-ICOS antibody, either alone or in combination with other agents, such as an anti-PD-L1 antibody.

### RELATED APPLICATIONS

This application is a continuation of International Patent Application No. PCT/GB2022/051413, filed Jun. 6, 2022, which claims priority to Great Britain Application No. 2107994.2, filed Jun. 4, 2021, the entire disclosures of which are hereby incorporated herein by reference in their entirety.

### SEQUENCE LISTING

The instant application contains a Sequence Listing which has been submitted electronically in XML format and is hereby incorporated by reference in its entirety. Said XML file, created on Mar. 5, 2024, is named 747461_SA9-646PCCON_ST26.xml and is 652,597 bytes in size.

### BACKGROUND

ICOS (Inducible T cell Co-Stimulator) is a member of the CD28 gene family involved in regulating immune responses, in particular humoral immune responses, first identified in 1999 [1]. It is a 55 kDa transmembrane protein, existing as a disulphide linked homodimer with two differentially glycosylated subunits. ICOS is exclusively expressed on T lymphocytes, and is found on a variety of T cell subsets. It is present at low levels on naïve T lymphocytes but its expression is rapidly induced upon immune activation, being upregulated in response to pro-inflammatory stimuli such as on engagement of TCR and co-stimulation with CD28 [2, 3]. ICOS plays a role in the late phase of T cell activation, memory T cell formation and importantly in the regulation of humoral responses through T cell dependent B cell responses [4, 5]. Intracellularly, ICOS binds PI3K and activates the kinases phophoinositide-dependent kinase 1 (PDK1) and protein kinase B (PKB). Activation of ICOS prevents cell death and upregulates cellular metabolism. In the absence of ICOS (ICOS knock-out) or in the presence of anti-ICOS neutralising antibodies there would be a suppression of pro-inflammatory responses.

ICOS binds to ICOS ligand (ICOSL) expressed on B-cells and antigen presenting cells (APC) [6, 7]. As a co-stimulatory molecule it serves to regulate TCR mediated immune responses and antibody responses to antigen. The expression of ICOS on T regulatory cells may be important, as it has been suggested that this cell type plays a negative role in immunosurveillance of cancer cells—there is emerging evidence for this in ovarian cancer [8]. Importantly, ICOS expression has been reported to be higher on intratumoural regulatory T cells (TRegs) compared with CD4+ and CD8+ effector cells that are present in the tumour microenvironment. Depletion of TRegs using antibodies with Fc-mediated cellular effector function has demonstrated strong anti-tumour efficacy in a pre-clinical model [9]. Mounting evidence implicates ICOS in an anti-tumour effect in both animal models as well as patients treated with immune-checkpoint inhibitors. In mice deficient in ICOS or ICOSL the anti-tumor effect of anti-CTLA4 therapy is diminished [10] while in normal mice ICOS ligand increases the effectiveness of anti-CTLA4 treatment in melanoma and prostate cancer [11]. Furthermore, in humans a retrospective study of advanced melanoma patients showed increased levels of ICOS following ipilimumab (anti-CTLA4) treatment [12]. In addition, ICOS expression is upregulated in bladder cancer patients treated with anti-CTLA4 [13]. It has also been observed that in cancer patients treated with anti-CTLA4 therapy the bulk of tumour specific IFNγ producing CD4 T-cells are ICOS positive while sustained elevation of ICOS positive CD4 T cells correlates with survival [12, 13, 14].

WO2016/120789 described anti-ICOS antibodies and proposed their use for activating T cells and for treating cancer, infectious disease and/or sepsis. A number of murine anti-ICOS antibodies were generated, of which a sub-set were reported to be agonists of the human ICOS receptor. The antibody “422.2” was selected as the lead anti-ICOS antibody and was humanised to produce a human “IgG4PE” antibody designated “H2L5”. H2L5 was reported to have an affinity of 1.34 nM for human ICOS and 0.95 nM for cynomolgus ICOS, to induce cytokine production in T cells, and to upregulate T cell activation markers in conjunction with CD3 stimulation. However, mice bearing implanted human melanoma cells were reported to show only minimal tumour growth delay or increase in survival when treated with H2L5 hIgG4PE, compared with control treated group. The antibody also failed to produce significant further inhibition of tumour growth in combination experiments with ipilimumab (anti-CTLA-4) or pembrolizumab (anti-PD-1), compared with ipilimumab or pembrolizumab monotherapy. Finally, In mice bearing implanted colon cancer cells (CT26), low doses of a mouse cross reactive surrogate of H2L5 in combination with a mouse surrogate of ipilimumab or pembrolizumab only mildly improved overall survival compared with anti-CTL4 and anti-PD1 therapy alone. A similar lack of strong therapeutic benefit was shown in mice bearing implanted EMT6 cells.

WO2016/154177 described further examples of anti-ICOS antibodies. These antibodies were reported to be agonists of CD4+ T cells, including effector CD8+ T cells (TEff), and to deplete T regulator cells (TRegs). Selective effects of the antibodies on TEff vs TReg cells were described, whereby the antibodies could preferentially deplete TRegs while having minimal effect on TEffs that express a lower level of ICOS. The anti-ICOS antibodies were proposed for use in treating cancer, and combination therapy with anti-PD-1 or anti-PD-L1 antibodies was described.

Programmed death-1 (PD-1) is a 50-55 kDa type I transmembrane receptor that is a member of the CD28 family. PD-1 is involved in the regulation of T-cell activation and is expressed on T-cells, B cells, and myeloid cells. Two ligands for PD-1, PD ligand 1 (PD-L1) and ligand 2 (PD-L2) have been identified and have co-stimulatory features.

Programmed cell death 1 ligand 1 (PD-L1), also known as cluster of differentiation (CD274) or B7 homolog 1 (B7-H1), is a member of the B7 family that modulates activation or inhibition of the PD-1 receptor. The open reading frame of PD-L1 encodes a putative type 1 transmembrane protein of 290 amino acids, which includes two extracellular Ig domains (a N-terminal V-like domain and a Ig C-like domain), a hydrophobic transmembrane domain and a cytoplasmic tail of 30 amino acids. The 30 amino acid intracellular (cytoplasmic) domain contains no obvious signalling motifs, but does have a potential site for protein kinase C phosphorylation.

The complete amino acid sequence for PD-L1 can be found in NCBI Reference Sequence: NP_054862.1 (SEQ ID NO: 1), which refers to many journal articles, including, for example, Dong, H., et al. (1999), “PD-L1, a third member of the B7 family, co-stimulates T-cell proliferation and interleukin-10 secretion,” Nat. Med. 5 (12), 1365-1369. The PD-L1 gene is conserved in chimpanzee, Rhesus monkey, dog, cow, mouse, rat, chicken, and zebrafish. The murine form of PD-L1 bears 69% amino acid identity with the human form of PD-L1, and also shares a conserved structure.

In humans, PD-L1 is expressed on a number of immune cell types including activated and anergic/exhausted T-cells, on naive and activated B-cells, as well as on myeloid dendritic cells (DC), monocytes and mast cells. It is also expressed on non-immune cells including islets of the pancreas, Kupffer cells of the liver, vascular endothelium and selected epithelia, for example airway epithelia and renal tubule epithelia, where its expression is enhanced during inflammatory episodes. PD-L1 expression is also found at increased levels on a number of tumours including, but not limited to breast (including but not limited to triple negative breast cancer and inflammatory breast cancer), ovarian, cervical, colon, colorectal, lung, including non-small cell lung cancer, renal, including renal cell carcinoma, gastric, oesophageal, bladder, hepatocellular cancer, squamous cell carcinoma of the head and neck (SCCHN) and pancreatic cancer, melanoma and uveal melanoma.

PD-1/PD-L1 signalling is believed to serve a critical non-redundant function within the immune system by negatively regulating T-cell responses. This regulation is involved in T-cell development in the thymus, in regulation of chronic inflammatory responses and in maintenance of both peripheral tolerance and immune privilege. It appears that upregulation of PD-L1 may allow cancers to evade the host immune system and, in many cancers, the expression of PD-L1 is associated with reduced survival and an unfavourable prognosis. Therapeutic monoclonal antibodies that are able to block the PD-1/PD-L1 pathway may enhance anti-tumoural immune responses in patients with cancer. Published clinical data suggest a correlation between clinical responses with tumoural membranous expression of PD-L1 (Brahmer et al., Journal of Clinical Oncology, 2010, Topalian et al., NEJM, 2012) and a stronger correlation between lack of clinical responses and a lack of PD-L1 protein localized to the membrane (Brahmer et al., Journal of Clinical Oncology, 2010, Topalian et al., NEJM, 2012). Thus, PD-L1 expression in tumours or tumour-infiltrating leukocytes (Herbst R S, et al., “Predictive correlates of response to the anti-PD-L1 antibody MPDL3280A in cancer patients”, Nature, 2014, Nov. 27, 515(7528):563-7, doi: 10.1038/nature14011) is a candidate molecular marker for use in selecting patients for immunotherapy, for example, immunotherapy using anti-PD-L1 antibodies. Patient enrichment based on surface expression of PD-L1 may significantly enhance the clinical success of treatment with drugs targeting the PD-1/PD-L1 pathway. There is also evidence of an on-going immune response, such as the tumour infiltrating CD8⁺ T-cells, or the presence of signature of cytokine activation, such as IFNγ.

Further evidence of PD-L1 expression and correlation to disease will emerge from the numerous ongoing clinical trials. Atezolizumab is the most advanced, and recent data from Phase II trials shows therapeutic effects in metastatic urothelial carcinoma and NSCLC, particularly in patients with PD-L1 immune cells in the tumour microenvironment (see Fehrenbacher et al., 2016, The Lancet, http://doi.org/10.1016/S0140-6736(16)00587-0; Rosenberg et al., 2016, The Lancet, http://doi.org/10.1016/S0140-6736(16)00561-4). Recent results from a Phase III trial of 1225 patients with NSCLC showed improved survival in patients taking atezolizumab, compared with chemotherapy, regardless of tumour expression of PD-L1 (Rittmeyer et al., 2017, The Lancet, 389(10066), 255-265).

WO2018/029474 describes exemplary anti-ICOS antibodies. WO2017/220990 described exemplary anti-PD-L1 antibodies.

PD-L1 expression is often used as a predictive marker for whether a tumour will respond treatment, such as a PD-L1 antibody. PD-L1 acts as a “brak on the immune system, in a negative feedback loop, to modulate the immune response. Although a suppressive signal, its presence in tumours is therefore indicative of an anti-tumour immune response. PD-L1 negative tumours are immunologically “cold”, their PD-L1 negative status indicating that the cells have not been exposed to inflammation. In general, higher PD-L1 expression is associated with greater inflammation and these PD-L1 high tumours are more likely to respond to immunotherapy, since there are pre-existing immune cells which are capable of “seeing” and attacking the tumour. Existing anti-PD-L1 antibodies that have been approved for treatment are approved only for PD-L1 expression tumours. It has previously been considered unlikely that PD-L1 low expressing tumours would response to immunotherapy, such as with an anti-ICOS antibody, an anti-PD-L1 antibody, or a combination of anti-ICOS and anti-PD-L1 antibodies.

### SUMMARY OF THE INVENTION

The present inventors have discovered that immunotherapy can successfully treat PD-L1-negative or PD-L1 low expressing tumours. More specifically, the present inventors have discovered that PD-L1-negative or PD-L1 low expressing tumours can be successfully treated with an inhibitor of ICOS (for example an anti-ICOS antibody or antigen binding fragment thereof) or with a combination of an ICOS inhibitor (for example an anti-ICOS antibody or antigen binding fragment thereof) and a PD-L1 inhibitor (for example an anti-PD-L1 antibody or antigen binding fragment thereof or an anti-PD-1 antibody or antigen binding fragment thereof). These treatments are surprising, as it was not previously considered possible to treat a PD-L1 negative or low PD-L1 expressing cancer with immunotherapy, in particular immunotherapy comprising administration of a PD-L1 inhibitor, such as an anti-PD-L1 antibody and/or comprising administration of an ICOS inhibitor, such as an anti-ICOS antibody. The present invention results in a surprising new mechanism for the treatment of cancers, including difficult-to-treat cancers, such as those with low levels of PD-L1 expression on the tumour cells and tumour-infiltrating lymphocytes, or PD-L1 negative cancers.

An antibody to ICOS that acts to increase effector T cell activity represents a therapeutic approach in immunooncology and in other medical contexts where a CD8+ T cell response is beneficial, including various diseases and conditions and in vaccination regimens. In many diseases and conditions involving an immune component, a balance exists between effector T cells (TEff) which exert the CD8+ T cell immune response, and regulatory T cells (TReg) which suppress that immune response by downregulating TEffs. The present invention relates to antibodies that modulate this TEff/TReg balance in favour of effector T cell activity. Antibodies that trigger the depletion of ICOS highly positive regulatory T cells would relieve the suppression of TEffs, and thus have a net effect of promoting the effector T cell response. An additional or complementary mechanism for an anti-ICOS antibody is via agonistic activity at the ICOS receptor level, to stimulate the effector T cell response.

The relative expression of ICOS on effector T cells (TEff) compared with regulatory T cells (TReg), and the relative activities of these cell populations, will influence the overall effect of an anti-ICOS antibody in vivo. An envisaged mode of action combines agonism of effector T cells with depletion of ICOS positive regulatory T cells. Differential and even opposing effects on these two different T cell populations may be achievable due to their different levels of ICOS expression. Dual-engineering of the variable and constant regions respectively of an anti-ICOS antibody can provide a molecule that exerts a net positive effect on effector T cell response by affecting the CD8/TReg ratio. An antigen-binding domain of an agonist antibody, which activates the ICOS receptor, may be combined with an antibody constant (Fc) region that promotes downregulation and/or clearance of highly expressing cells to which the antibody is bound. An effector positive constant region may be used to recruit cellular effector functions against the target cells (TRegs), e.g., to promote antibody-dependent cell-mediated cytotoxicity (ADCC) or antibody dependent cell phagocytosis (ADCP). The antibody may thus act both to promote effector T cell activation and to downregulate immunosuppressive T Regulatory cells.

Since ICOS is more highly expressed on TRegs than on TEffs, a therapeutic balance may be achieved whereby Teff function is promoted while TRegs are depleted, resulting in a net increase in the T cell immune response (e.g, anti-tumour response or other therapeutically beneficial T cell response).

Several pre-clinical and clinical studies have shown a strong positive correlation between high effector T-cell to T-reg cell ratio in the tumour microenvironment (TME) and overall survival. In ovarian cancer patients the ratio of CD8:T-reg cells has been reported to be an indicator of good clinical outcome [15]. A similar observation was made in metastatic melanoma patients after receiving ipilumumab [16]. In pre-clinical studies, it has also been shown that high effector cell:T-reg ratio in TME is associated with anti-tumour response.

This invention uses antibodies that bind human ICOS. The antibodies target the ICOS extracellular domain and thereby bind to T cells expressing ICOS. Examples are provided of antibodies that have been designed to have an agonistic effect on ICOS, thus enhancing the function of effector T cells, as indicated by an ability to increase IFNγ expression and secretion. As noted, anti-ICOS antibodies may also be engineered to deplete cells to which they bind, which should have the effect of preferentially downregulating regulatory T cells, lifting the suppressive effect of these cells on the effector T cell response and thus promoting the effector T cell response overall. Regardless of their mechanism of action, it is demonstrated empirically that anti-ICOS antibodies according to the present invention do stimulate T cell response and have anti-tumour effects in vivo, as shown in the Examples. Through selection of appropriate antibody formats such as those including constant regions with a desired level of Fc effector function, or absence of such effector function where appropriate, the anti-ICOS antibodies may be tailored for use in a variety of medical contexts including treatment of diseases and conditions in which an effector T cell response is beneficial and/or where suppression of regulatory T cells is desired.

Exemplary anti-ICOS antibodies include STIM001, STIM002, STIM002-B, STIM003, STIM004, STIM005, STIM006, STIM007, STIM008 and STIM009, the sequences of which are set out herein.

The present invention provides a method of treating cancer in a patient, wherein the patient has a PD-L1 negative tumour or a tumour with low PD-L1 expression, comprising administering to the patient an modulator of ICOS.

The present invention also provides a method of treating cancer in a patient who has previously received treatment for the cancer, wherein the previous treatment for the cancer was administration of a PD-L1 inhibitor and the patient did not respond to the previous treatment or ceased responding to the previous treatment, comprising administering to the patient a modulator of ICOS.

The present invention also provides an ICOS modulator for use in the treatment of cancer in a patient, wherein the patient has a PD-L1 negative tumour or a tumour with low PD-L1 expression.

The present invention also provides an ICOS modulator for use in the treatment of cancer in a patient, wherein the patient has previously received treatment for the cancer and the patient did not respond to the previous treatment or ceased responding to the previous treatment, wherein the previous treatment for the cancer was a PD-L1 inhibitor.

The present invention also provides use of an ICOS modulator in the manufacture of a medicament for the treatment of cancer in a patient, wherein the patient has a PD-L1 negative tumour or a tumour with low PD-L1 expression.

The present invention also provides use of an ICOS modulator in the manufacture of a medicament for the treatment of cancer in a patient, wherein the patient has previously received treatment for the cancer and the patient did not respond to the previous treatment or ceased responding to the previous treatment, wherein the previous treatment for the cancer was a PD-L1 inhibitor.

Generally, the modulator of ICOS is an ICOS agonist. The modulator of ICOS may be an anti-ICOS antibody. In preferred embodiments, the modulator of ICOS is an agonistic anti-ICOS antibody.

In some embodiments, the methods or uses may involve a combination therapy with a PD-L1 inhibitor, for example a PD-L1 inhibitor that prevents the binding of PD-L1 to PD-1, such as an anti-PD-L1 or and anti-PD-1 antibody.

An anti-ICOS antibody used in the invention may be one that competes for binding to human ICOS with an antibody (e.g., human IgG1, or an scFv) comprising the heavy and light chain complementarity determining regions (CDRs) of STIM001, STIM002, STIM002-B, STIM003, STIM004, STIM005, STIM006, STIM007, STIM008 or STIM009, optionally an antibody comprising the VH and VL domains of STIM001, STIM002, STIM002-B, STIM003, STIM004, STIM005, STIM006, STIM007, STIM008 or STIM009.

An anti-ICOS antibody according to the present invention may comprise one or more CDRs of any of STIM001, STIM002, STIM002-B, STIM003, STIM004, STIM005, STIM006, STIM007, STIM008 and STIM009 (e.g., all 6 CDRs of any such antibody, or a set of HCDRs and/or LCDRs) or variants thereof as described herein.

The anti-ICOS antibody may comprise an antibody VH domain comprising CDRs HCDR1, HCDR2 and HCDR3 and an antibody VL domain comprising CDRs LCDR1, LCDR2 and LCDR3, wherein the HCDR3 is an HCDR3 of an antibody selected from STIM001, STIM002, STIM002-B, STIM003, STIM004, STIM005, STIM006, STIM007, STIM008 and STIM009 or comprises that HCDR3 with 1, 2, 3, 4 or 5 amino acid alterations. The HCDR2 may be the HCDR2 of the selected antibody or it may comprise that HCDR2 with 1, 2, 3, 4 or 5 amino acid alterations. The HCDR1 may be the HCDR1 of the selected antibody or it may comprise that HCDR1 with 1, 2, 3, 4 or 5 amino acid alterations.

The anti-ICOS antibody may comprise an antibody VL domain comprising CDRs HCDR1, HCDR2 and HCDR3 and an antibody VL domain comprising CDRs LCDR1, LCDR2 and LCDR3, wherein the LCDR3 is an LCDR3 of an antibody selected from STIM001, STIM002, STIM002-B, STIM003, STIM004, STIM005, STIM006, STIM007, STIM008 and STIM009 or comprises that LCDR3 with 1, 2, 3, 4 or 5 amino acid alterations. The LCDR2 may be the LCDR2 of the selected antibody or it may comprise that LCDR2 with 1, 2, 3, 4 or 5 amino acid alterations. The LCDR1 may be the LCDR1 of the selected antibody or it may comprise that LCDR1 with 1, 2, 3, 4 or 5 amino acid alterations.

An anti-ICOS antibody may comprise:
an antibody VH domain comprising complementarity determining regions HCDR1, HCDR2 and HCDR3, and
an antibody VL domain comprising complementarity determining regions LCDR1, LCDR2 and LCDR3,
wherein the heavy chain complementarity determining regions are those of STIM001, STIM002, STIM002-B, STIM003, STIM004, STIM005, STIM006, STIM007, STIM008 or STIM009 or comprise the STIM001, STIM002, STIM002-B, STIM003, STIM004 or STIM005, STIM006, STIM007, STIM008 or STIM009 heavy chain complementarity determining regions with 1, 2, 3, 4 or 5 amino acid alterations; and/or
wherein the light chain complementarity determining regions are those of antibody STIM001, STIM002, STIM002-B, STIM003, STIM004, STIM005, STIM006, STIM007, STIM008 or STIM009, or comprise the STIM001, STIM002, STIM002-B, STIM003, STIM004, STIM005, STIM006, STIM007, STIM008 or STIM009 light chain complementarity determining regions with 1, 2, 3, 4 or 5 amino acid alterations.

An anti-ICOS antibody may comprise a VH domain comprising a set of heavy chain complementarity determining regions (HCDRs) HCDR1, HCDR2 and HCDR3, wherein
HCDR1 is the HCDR1 of STIM003,
HCDR2 is the HCDR2 of STIM003,
HCDR3 is the HCDR3 of STIM003,
or comprising that set of HCDRs with 1, 2, 3, 4, 5 or 6 amino acid alterations.

An anti-ICOS antibody may comprise a VL domain comprising a set of light chain complementarity determining regions (LCDRs) LCDR1, LCDR2 and LCDR3, wherein
LCDR1 is the LCDR1 of STIM003,
LCDR2 is the LCDR2 of STIM003,
LCDR3 is the LCDR3 of STIM003,
or comprising that set of LCDRs with 1, 2, 3 or 4 amino acid alterations.

Amino acid alterations (e.g., substitutions) may be at any residue position in the CDRs. Examples of amino acid alterations are those illustrated in FIG. 10, FIG. 11 and FIG. 12, which show alignments of variant sequences of anti-ICOS antibodies. Thus, an amino acid alteration in a STIM003 CDR may be a substitution of the residue present at the corresponding position in antibody CL-74570 or antibody CL-71642 as indicated in FIG. 11.

Example amino acid alterations in STIM003 CDRs are substitutions at the following residue positions, defined according to IMGT:
In HCDR1, substitution at IMGT position 28, optionally a conservative substitution, e.g., V28F.
In HCDR2, substitution at IMGT position 59, 63 and/or 64. Optionally the substitution at position 59 is N591, the substitution at position 63 is G63D and/or the substitution at position 64 is D64N and/or D64S.
In HCDR3, substitution at IMGT position 106, 108, 109 and/or 112. Optionally the substitution at position 106 is R106A, the substitution at position 108 is F108Y, the substitution at position 109 is Y109F and/or the substitution at position 112 is H112N.
In LCDR1, substitution at position 36, e.g., R36S.
In LCDR3, substitution at position 105, 108 and/or 109. Optionally the substitution at position 105 is H105Q, the substitution at position 108 is D108G and/or the substitution at position 109 is M109N or M109S.

Anti-ICOS antibodies used in the invention may comprise VH and/or VL domain framework regions corresponding to human germline gene segment sequences. For example, it may comprise one or more framework regions of STIM001, STIM002, STIM002-B, STIM003, STIM004, STIM005, STIM006, STIM007, STIM008 or STIM009. The framework region or framework regions may be a FR1, FR2, FR3 and/or FR4.

As described in Example 2, Table E12-1 shows the human germline V, D and J gene segments that generated the VH domains of these antibodies through recombination and Table E12-2 shows the human germline V and J gene segments that generated the VL domains of these antibodies through recombination. Antibody VH and VL domains used in the present invention may be based on these V(D)J segments.

An antibody used in the invention may comprise an antibody VH domain which
(i) is derived from recombination of a human heavy chain V gene segment, a human heavy chain D gene segment and a human heavy chain J gene segment, wherein
   the V segment is IGHV1-18 (e.g., V1-18*01), IGVH3-20 (e.g. V3-20*d01), IGVH3-11 (e.g, V3-11*01) or IGVH2-5 (e.g., V2-5*10);
   the D gene segment is IGHD6-19 (e.g., IGHD6-19*01), IGHD3-10 (e.g., IGHD3-10*01) or IGHD3-9 (e.g., IGHD3-9*01); and/or
   the J gene segment is IGHJ6 (e.g., IGHJ6*02), IGHJ4 (e.g., IGHJ4*02) or IGHJ3 (e.g., IGHJ3*02), or
(ii) comprises framework regions FR1, FR2, FR3 and FR4, wherein
   FR1 aligns with human germline V gene segment IGHV1-18 (e.g., V1-18*01), IGVH3-20 (e.g. V3-20*d01), IGVH3-11 (e.g, V3-11*01) or IGVH2-5 (e.g., V2-5*10), optionally with 1, 2, 3, 4 or 5 amino acid alterations,
   FR2 aligns with human germline V gene segment IGHV1-18 (e.g., V1-18*01), IGVH3-20 (e.g. V3-20*d01), IGVH3-11 (e.g, V3-11*01) or IGVH2-5 (e.g., V2-5*10), optionally with 1, 2, 3, 4 or 5 amino acid alterations,
   FR3 aligns with human germline V gene segment IGHV1-18 (e.g., V1-18*01), IGVH3-20 (e.g. V3-20*d01), IGVH3-11 (e.g, V3-11*01) or IGVH2-5 (e.g., V2-5*10), optionally with 1, 2, 3, 4 or 5 amino acid alterations, and/or
   FR4 aligns with human germline J gene segment IGJH6 (e.g., JH6*02), IGJH4 (e.g., JH4*02) or IGJH3 (e.g., JH3*02), optionally with 1, 2, 3, 4 or 5 amino acid alterations.

FR1, FR2 and FR3 of the VH domain typically align with the same germline V gene segment. Thus, for example, the antibody may comprise a VH domain derived from recombination of human heavy chain V gene segment IGHV3-20 (e.g., VH3-20*d01), a human heavy chain D gene segment and a human heavy chain J gene segment IGJH4 (e.g., JH4*02). An antibody may comprise VH domain framework regions FR1, FR2, FR3 and FR4, wherein FR1, FR2 and FR3 each align with human germline V gene segment IGHV3-20 (e.g., IGVH3-20*d01) with up to 1, 2, 3, 4 or 5 amino acid alterations, and a FR4 that aligns with human germline J gene segment IGHJ4 (e.g., IGHJ4*02) with up to 1, 2, 3, 4 or 5 amino acid alterations. Alignment may be exact, but in some cases one or more residues can be mutated from germline, so there may be amino acid substitutions present, or in rarer cases deletions or insertions.

An antibody used in the invention may comprise an antibody VL domain which
(i) is derived from recombination of a human light chain V gene segment and a human light chain J gene segment, wherein
   the V segment is IGKV2-28 (e.g., IGKV2-28*01), IGKV3-20 (e.g., IGKV3-20*01), IGKV1D-39 (e.g., IGKV1D-39*01) or IGKV3-11 (e.g., IGKV3-11*01), and/or
   the J gene segment is IGKJ4 (e.g., IGKJ4*01), IGKJ2 (e.g., IGKJ2*04), IGLJ3 (e.g., IGKJ3*01) or IGKJ1 (e.g., IGKJ1*01); or
(ii) comprises framework regions FR1, FR2, FR3 and FR4, wherein
   FR1 aligns with human germline V gene segment IGKV2-28 (e.g., IGKV2-28*01), IGKV3-20 (e.g., IGKV3-20*01), IGKV1D-39 (e.g., IGKV1D-39*01) or IGKV3-11 (e.g., IGKV3-11*01), optionally with 1, 2, 3, 4 or 5 amino acid alterations,
   FR2 aligns with human germline V gene segment IGKV2-28 (e.g., IGKV2-28*01), IGKV3-20 (e.g., IGKV3-20*01), IGKV1D-39 (e.g., IGKV1D-39*01) or IGKV3-11 (e.g., IGKV3-11*01), optionally with 1, 2, 3, 4 or 5 amino acid alterations,
   FR3 aligns with human germline V gene segment IGKV2-28 (e.g., IGKV2-28*01), IGKV3-20 (e.g., IGKV3-20*01), IGKV1D-39 (e.g., IGKV1D-39*01) or IGKV3-11 (e.g., IGKV3-11*01), optionally with 1, 2, 3, 4 or 5 amino acid alterations, and/or
   FR4 aligns with human germline J gene segment IGKJ4 (e.g., IGKJ4*01), IGKJ2 (e.g., IGKJ2*04), IGKJ3 (e.g., IGKJ3*01) or IGKJ1 (e.g., IGKJ1*01), optionally with 1, 2, 3, 4 or 5 amino acid alterations.

FR1, FR2 and FR3 of the VL domain typically align with the same germline V gene segment. Thus, for example, the antibody may comprise a VL domain derived from recombination of human light chain V gene segment IGKV3-20 (e.g., IGKV3-20*01) and human light chain J gene segment IGKJ3 (e.g., IGKJ3*01). An antibody may comprise VL domain framework regions FR1, FR2, FR3 and FR4, wherein FR1, FR2 and FR3 each align with human germline V gene segment IGKV3-20 (e.g., IGKV3-20*01) with up to 1, 2, 3, 4 or 5 amino acid alterations, and a FR4 that aligns with human germline J gene segment IGKJ3 (e.g., IGKJ3*01) with up to 1, 2, 3, 4 or 5 amino acid alterations. Alignment may be exact, but in some cases one or more residues can be mutated from germline, so there may be amino acid substitutions present, or in rarer cases deletions or insertions.

An antibody used in the invention may comprise an antibody VH domain which is the VH domain of STIM001, STIM002, STIM002-B, STIM003, STIM004 or STIM005, STIM006, STIM007, STIM008 or STIM009, or which has an amino acid sequence at least 90% identical to the antibody VH domain sequence of STIM001, STIM002, STIM002-B, STIM003, STIM004, STIM005, STIM006, STIM007, STIM008 or STIM009. The amino acid sequence identity may be at least 95%.

The antibody may comprise an antibody VL domain which is the VL domain of STIM001, STIM002, STIM002-B, STIM003, STIM004 or STIM005, STIM006, STIM007, STIM008 or STIM009, or which has an amino acid sequence at least 90% identical to the antibody VL domain sequence of STIM001, STIM002, STIM002-B, STIM003, STIM004, STIM005, STIM006, STIM007, STIM008 or STIM009. The amino acid sequence identity may be at least 95%.

An antibody VH domain having the HCDRs of STIM001, STIM002, STIM002-B, STIM003, STIM004, STIM005, STIM006, STIM007, STIM008 or STIM009, or having a variant of those CDRs, may be paired with an antibody VL domain having the LCDRs of the same antibody, or having a variant of those CDRs. Similarly, the VH domain of any of STIM001, STIM002, STIM002-B, STIM003, STIM004, STIM005, STIM006, STIM007, STIM008 or STIM009, or a variant of that VH domain, may be paired with a VL domain of the same antibody, or a VL domain variant of the same antibody.

For instance, the antibody may comprise the antibody STIM001 VH domain and the STIM001 VL domain. In another example, the antibody may comprise the antibody STIM002 VH domain and the STIM002 VL domain. In another example, the antibody may comprise the antibody STIM003 VH domain and the STIM003 VL domain.

Antibodies may include constant regions, optionally human heavy and/or light chain constant regions. An exemplary isotype is IgG, e.g., human IgG1.

### BRIEF DESCRIPTION OF THE DRAWINGS

Certain aspects and embodiments of the invention will now be described in more detail with reference to the accompanying drawings.

FIG. 1: Dual mechanism of action of KY1044 (aka STIM003), an agonistic anti-ICOS antibody. (A) Tumour untreated prior to KY1044 treatment. (i) Suppression of Effector T cells (ii) Regulatory T cell-mediated immune evasion. (B) Tumor and ICOS agonism. (iii) ICOS^{HI} Regulatory T cells remain. (iv) ICOS^{LOW} Effector T cells stimulated, IFNγ expressed. (C) Tumor and depletion ICOS^{HI} T_{regs}. (v) ICOS^{HI} Regulatory T cells killed. This figure shows the potential for a dula mechanism of action (agonism and depletion). One aspect of the dual mechanism of action is ICOS T_{eff} agonism, which increases activation (cytokine production) of T_{eff} cells, as shown in (B). The other aspect of the dual mechanism of action is ICOS T_{reg} depletion which releases the inhibition of T_{eff} cells, as shown in (C).

FIG. 2: Schematic of a clinical trial study design. All corner patient population with preferred indications: e.g. NSCLC, HNSCC, HCC, melanoma cervical, gatric/esophageal, renal, pancreatic and TNBC. Dosing: Q3W I.V. (**=n=21 enrolled, n=20 treated) (i) KY1044 and enrichment pool. (ii) KY1044+Atezolizumab and enrichment pool. Phase 1 Dose Escalation (completed)—KY1044 single agent dose escalation, and—KY1044 in combination with atezolizumab dose escalation. Phase 1 Enrichment Cohorts (ongoing). Phase 2 Expansion (ongoing)—selected indications in which anti-tumor activity was observed in Phase 1.

FIG. 3: Staining of cells for PD-L1 expression

FIG. 4: Threshold of the CD8 low vs high based on the median. (A) PD-L1⁺ immune infiltrate in the TME and CD8⁺ in the TME. (B) PD-L1⁺ on tumour cells in TME and CD8⁺ in the TME. Each panel (A) and (B) is divided into four quadrants: Q1=CD8 low/PD-L1 high; Q2=Hot tumor and PD-L1 high; Q3=Cold tumor; Q4=CD8 high/PD-L1 low. PR=partial response, CR=complete response, SD=stable disease, PD=progressive disease.

FIG. 5: Effect of anti-ICOS treatment using KY1044 in Patient A. (A) Information table for Patient A. (B) TME analysis (as determined by IHC) at screening at C2D8 (cycle 2, day 8), (i) Minimal effect on CD8⁺ T cells. (ii) Depletion of ICOS⁺ Tregs. (iii) 73.6-fold improvement in CD8⁺/ICOS⁺ T_{reg} ratio. (C) PD-L1 expression in the TME (as determined by IHC) at screening at C2D8, (iv) 0% PD-L1⁺ tumor cells. (v) Low PD-L1⁺ immune infiltrate in TME. (D) Baseline PBMC analysis (as determined by chipcytometry), (vi) Low CD4 cells; High CD8 cells % of T cells. (vii) average T cells; above average monocytes. (viii) above average % of ICOS⁺ cells. (E) Longitudinal PBCM and ICOS RO analysis (as determined by chipcytometry) (pre dose, cycle 1 day 8, cycle 2 day 1 (pre dose) and cycle 2 day 8). (ix) No depletion of peripheral CD4 memory cells. (x) No free ICOS on peripheral CD4 MEMs.

FIG. 6: IHC analysis for Patient A at screening and at C2D8 following treatment with KY1044. (A) Depletion of ICOS⁺ T_{regs} 75.97 to 0.7 (cells/mm²) at screening and C2D8. (B) CD8⁺ cells present 227.27 and 154.11 (cells/mm²) at screening and C2D8. (C) PD-L1 tumor 0% at both timepoints, PD-L1 infiltrate 1% and 0% at screening and C2D8.

FIG. 7: Effect of anti-ICOS treatment using KY1044 in Patient B. (A) Information table for Patient B. (B) TME analysis (as determined by IHC). (i) Average density of CD8⁺ T cells at screening. (ii) Very low density of ICOS⁺ T_{regs} at screening. (iii) Very high ratio of CD8⁺/ICOS⁺ T_{regs} at screening. (C) PD-L1 expression in the TME (as determined by IHC). (iv) 0% PD-L1 tumor cells at screening. (v) Low PD-L1⁺ immune infiltrate in TME. (D) Longitudinal PBMC and ICOS RO analysis in patient B (as determined by chipcytometry). (vi) No depletion of peripheral CD4 memory cells. (vii) No free ICOS on peripheral CD4 MEMs. This patient reached a stable disease state following treatment with KY1044.

FIG. 8: IHC analysis for Patient B at screening. (A) Very low ICOS⁺ T_{reg} density at screening 0.07 (cells/mm²). (B) High CD8⁺ cell density at screening 98.65 (cells/mm²). (C) PD-L1⁺ tumor 0% at screening, PD-L1 infiltrate 0% at screening.

FIG. 9: Patient Case Study—Patient C. Results for Patient C following treatment with KY1044, showing reduction in target lesion size at C3D8 and C10D1. Patient C information: Age/sex/diagnosis=59 years/male/HPV positive metastatic squamous cell carcinoma of the head/neck. PD-L1 status at screening (SP263): (% TC/% IC)=3/2. Allocation=KY1044 8.0 mg+atezolizumab 1,200 mg Q3W. (A) Previous therapies vs times on treatment in this study. 5-FU=fluorouracil, PD=disease progression, PR=partial response, **=patient was maintaining response of PR at the data cut for this figure (16 Dec. 2020). (B) Target lesion change from baseline. (C) Baseline (June 2020). (D) Cycle 3/Day 8 (August 2020).

FIG. 10: STIM002 VH (top) and VL (bottom) domain amino acid sequences, showing residues that differ in the corresponding sequences of STIM001, STIM002B and related antibodies CL-61091, CL-64536, CL-64837, CL-64841 and CL-64912 and/or in the human germline. Sequence numbering is according to IMGT.

FIG. 11: STIM003 VH (top) and VL (bottom) domain amino acid sequences, showing residues that differ in the corresponding sequences of related antibodies CL-71642 and CL-74570 and/or in the human germline. Sequence numbering is according to IMGT. The VL domain of antibody CL-71642 obtained from sequencing is shown here without the N terminal residue. From the alignment it can be seen that the full VH domain sequence would comprise an N terminal glutamic acid.

FIG. 12: STIM007 VH (top) and VL (bottom) domain amino acid sequences, showing residues that differ in the corresponding sequences of STIM008 and/or in the human germline. Sequence numbering is according to IMGT.

### DETAILED DESCRIPTION

### ICOS

Anti-ICOS antibodies used in the present invention bind the extracellular domain of human ICOS. Thus, the antibodies bind ICOS-expressing T lymphocytes. “ICOS” or “the ICOS receptor” referred to herein may be human ICOS, unless the context dictates otherwise. Sequences of human, cynomolgus and mouse ICOS are shown in the appended sequence listing, and are available from NCBI as human NCBI ID: NP_036224.1, mouse NCBI ID: NP_059508.2 and cynomolgus GenBank ID: EHH55098.1.

### PD-L1

Many tumour cells express surface molecules that are specific to cancer that can serve as diagnostic and/or therapeutic antibody targets. Examples of cell surface proteins expressed by tumour molecules that can be useful as biomarkers include, for example, members of the B7 family of proteins, major histocompatibility complex molecules (MHC), cytokine and growth factor receptors such as the receptor for epidermal growth factor (EGFR). The B7 family is a group of proteins that are members of the immunoglobulin (Ig) superfamily of cell-surface proteins that bind to receptors on lymphocytes to regulate immune responses. The family includes transmembrane or glycosylphosphatidylinositol (GPI)-linked proteins characterized by extracellular Ig-like domains (IgV and IgC domains related to the variable and constant domains of immunoglobulins). All members have short cytoplasmic domains. There are seven known members of the B7 family: B7-1, B7-2, PD-L1 (B7-H1), PD-L2, B7-H2, B7-H3, and B7-H4.

The complete amino acid sequence for PD-L1 can be found in NCBI Reference Sequence: NP 054862.1, which refers to many journal articles, including, for example, Dong, H., et al. (1999), “PD-L1, a third member of the B7 family, co-stimulates T-cell proliferation and interleukin-10 secretion,” Nat. Med. 5 (12), 1365-1369, the disclosure of which is hereby incorporated by reference herein in its entirety. The amino acid sequence of PD-L1 includes a 30 amino acid long cytoplasmic domain that is unique to PD-L1, which shows little homology to other molecules, including other B7 family members.

### PD-1

The complete amino acid sequence for PD-1 can be found UniProt accession no. Q9UMF3.

### ICOS Modulators

The ICOS modulators used in the present invention may be any suitable ICOS modulators. Generally, the ICOS modulator may be an ICOS agonist. In some embodiments, the ICOS modulator is an anti-ICOS antibody. In preferred embodiments, the ICOS modulator is an agonistic anti-ICOS antibody.

The ICOS modulators (for example agonistic anti-ICOS antibodies) may deplete ICOS+ T Cells, in particular ICOS+ Tregs.

In some embodiments, the ICOS modulators are multispecific (such as bispecific), that is they specifically bind to multiple (for example two) different antigens. In some embodiments, the ICOS modulator is a multi-specific antibody (for example a bispecific antibody) that specifically binds ICOS and PD-L1 or PD-1. In some embodiments, the ICOS modulator is a multi-specific antibody (for example a bispecific antibody) that specifically binds ICOS and is an ICOS agonist, and specifically binds PD-L1 or PD-1 and is a PD-L1 or PD-1 antagonist.

### PD-L1 Inhibitors

The PD-L1 inhibitors used in the present invention generally inhibit the binding of PD-L1 to PD-1 (or the binding of PD-1 to PD-L1). The PD-L1 inhibitors may be an anti-PD-L1 or anti-PD-1 binding molecule. In some embodiments, the PD-L1 or PD-1 inhibitors are anti-PD-L1 or anti-PD-1 antibodies, respectively. Generally, the PD-L1 inhibitors are antagonists of PD-L1, for example antagonistic anti-PD-L1 or anti-PD-1 antibodies.

### Combinations of ICOS Modulators and PD-L1 Inhibitors

In some embodiments the invention uses a combination of an ICOS modulator and a PD-L1 inhibitor. The ICOS modulator and PD-L1 inhibitor may be for simultaneous, separate or sequential administration. In some embodiments, the ICOS modulator is an anti-ICOS antibody (for example an agonistic anti-ICOS antibody) and the PD-L1 inhibitor is an anti-PD-L1 antibody or an anti-PD-1 antibody. In some embodiments, the ICOS modulator is an IgG1 anti-ICOS antibody and the PD-L1 inhibitor is an IgG1 anti-PD-L1 antibody or an IgG1 anti-PD-1 antibody

### Cross-Reactivity

Antibodies used in the present invention are preferably cross-reactive, and may for example bind the extracellular domain of mouse ICOS as well as human ICOS. The antibodies may bind other non-human ICOS, including ICOS of primates such as cynomolgus. An anti-ICOS antibody intended for therapeutic use in humans must bind human ICOS, whereas binding to ICOS of other species would not have direct therapeutic relevance in the human clinical context. Nevertheless, the data herein indicate that antibodies that bind both human and mouse ICOS have properties that render them particularly suitable as agonist and depleting molecules. This may result from one or more particular epitopes being targeted by the cross-reactive antibodies. Regardless of the underlying theory, however, cross-reactive antibodies are of high value and are excellent candidates as therapeutic molecules for pre-clinical and clinical studies. Anti-PD-L1 and/or anti-PD-1 antibodies used in the invention may also exhibit cross-reactivity.

The STIM antibodies described here were generated using Kymouse™ technology where the mouse had been engineered to lack expression of mouse ICOS (an ICOS knock-out). ICOS knock-out transgenic animals and their use for generating cross-reactive antibodies are further aspects of the present invention.

One way to quantify the extent of species cross-reactivity of an antibody is as the fold-difference in its affinity for antigen or one species compared with antigen of another species, e.g., fold difference in affinity for human ICOS vs mouse ICOS. Affinity may be quantified as K_{D}, referring to the equilibrium dissociation constant of the antibody-antigen reaction as determined by SPR with the antibody in Fab format as described elsewhere herein. A species cross-reactive anti-ICOS antibody may have a fold-difference in affinity for binding human and mouse ICOS that is 30-fold or less, 25-fold or less, 20-fold or less, 15-fold or less, 10-fold or less or 5-fold or less. To put it another way, the K_{D} of binding the extracellular domain of human ICOS may be within 30-fold, 25-fold, 20-fold, 15-fold, 10-fold or 5-fold of the K_{D} of binding the extracellular domain of mouse ICOS. Antibodies can also be considered cross-reactive if the K_{D} for binding antigen of both species meets a threshold value, e.g., if the K_{D} of binding human ICOS and the K_{D} of binding mouse ICOS are both 10 mM or less, preferably 5 mM or less, more preferably 1 mM or less. The K_{D} may be 10 nM or less, 5 nM or less, 2 nM or less, or 1 nM or less. The K_{D} may be 0.9 nM or less, 0.8 nM or less, 0.7 nM or less, 0.6 nM or less, 0.5 nM or less, 0.4 nM or less, 0.3 nM or less, 0.2 nM or less, or 0.1 nM or less.

An alternative measure of cross-reactivity for binding human ICOS and mouse ICOS is the ability of an antibody to neutralise ICOS ligand binding to ICOS receptor, such as in an HTRF assay (see Example 8 of WO2018/029474). Examples of species cross-reactive antibodies are provided herein, including STIM001, STIM002, STIM002-B, STIM003, STIM005 and STIM006, each of which was confirmed as neutralising binding of human B7-H2 (ICOS ligand) to human ICOS and neutralising binding of mouse B7-H2 to mouse ICOS in an HTRF assay. Any of these antibodies or their variants may be selected when an antibody cross-reactive for human and mouse ICOS is desired. A species cross-reactive anti-ICOS antibody may have an IC50 for inhibiting binding of human ICOS to human ICOS receptor that is within 25-fold, 20-fold, 15-fold, 10-fold or 5-fold of the IC50 for inhibiting mouse ICOS to mouse ICOS receptor as determined in an HTRF assay. Antibodies can also be considered cross-reactive if the IC50 for inhibiting binding of human ICOS to human ICOS receptor and the IC50 for inhibiting binding of mouse ICOS to mouse ICOS receptor are both 1 mM or less, preferably 0.5 mM or less, e.g., 30 nM or less, 20 nM or less, 10 nM or less. The IC50s may be 5 nM or less, 4 nM or less, 3 nM or less or 2 nM or less. In some cases the IC50s will be at least 0.1 nM, at least 0.5 nM or at least 1 nM.

### Specificity

Antibodies used according to the present invention are preferably specific for ICOS. That is, the antibody binds its epitope on the target protein, ICOS (human ICOS, and preferably mouse and/or cynomolgus ICOS as noted above), but does not show significant binding to molecules that do not present that epitope, including other molecules in the CD28 gene family. An antibody according to the present invention preferably does not bind human CD28. The antibody preferably also does not bind mouse or cynomolgus CD28.

CD28 co-stimulates T cell responses when engaged by its ligands CD80 and CD86 on professional antigen presenting cells in the context of antigen recognition via the TCR. For various in vivo uses of the antibodies described herein, the avoidance of binding to CD28 is considered advantageous. Non-binding of the anti-ICOS antibody to CD28 should allow CD28 to interact with its native ligands and to generate appropriate co-stimulatory signal for T cell activation. Additionally, non-binding of the anti-ICOS antibody to CD28 avoids the risk of superagonism. Over-stimulation of CD28 can induce proliferation in resting T cells without the normal requirement for recognition of a cognate antigen via the TCR, potentially leading to runaway activation of T cells and consequent cytokine-release syndrome, especially in human subjects. The non-recognition of CD28 by antibodies according to the present invention therefore represents an advantage in terms of their safe clinical use in humans.

As discussed elsewhere herein, the present invention extends to multispecific antibodies (e.g., bispecifics). A multispecific (e.g., bispecific) antibody may comprise (i) an antibody antigen binding site for ICOS and (ii) a further antigen binding site (optionally an antibody antigen binding site, as described herein) which recognises another antigen (e.g., PD-L1). Specific binding of individual antigen binding sites may be determined. Thus, antibodies that specifically bind ICOS include antibodies comprising an antigen binding site that specifically binds ICOS, wherein optionally the antigen binding site for ICOS is comprised within an antigen-binding molecule that further includes one or more additional binding sites for one or more other antigens, e.g., a bispecific antibody that binds ICOS and PD-L1.

Some antibodies used in the invention specifically bind PD-L1 or PD-1. That is, the antibody binds its epitope on the target protein, PD-L1 or PD-1 (human PD-L1 or PD-1, and preferably mouse and/or cynomolgus PD-L1 or PD-1), but does not show significant binding to molecules that do not present that epitope.

### Affinity

The affinity of binding of an antibody to ICOS (or to another antigen, such as PD-L1 or PD-1) may be determined. Affinity of an antibody for its antigen may be quantified in terms of the equilibrium dissociation constant K_{D}, the ratio Ka/Kd of the association or on-rate (Ka) and the dissociation or off-rate (kd) of the antibody-antigen interaction. Kd, Ka and Kd for antibody-antigen binding can be measured using surface plasmon resonance (SPR).

An antibody used in the present invention may bind the EC domain of human ICOS with a K_{D} of 10 mM or less, preferably 5 mM or less, more preferably 1 mM or less. The K_{D} may be 50 nM or less, 10 nM or less, 5 nM or less, 2 nM or less, or 1 nM or less. The K_{D} may be 0.9 nM or less, 0.8 nM or less, 0.7 nM or less, 0.6 nM or less, 0.5 nM or less, 0.4 nM or less, 0.3 nM or less, 0.2 nM or less, or 0.1 nM or less. The K_{D} may be at least 0.001 nM, for example at least 0.01 nM or at least 0.1 nM.

Quantification of affinity may be performed using SPR with the antibody in Fab format. A suitable protocol is as follows:
1. Coupling anti-human (or other antibody constant region species-matched) IgG to a biosensor chip (e.g., GLM chip) such as by primary amine coupling;
2. Exposing the anti-human IgG (or other matched species antibody) to a test antibody, e.g., in Fab format, to capture test antibody on the chip;
3. Passing the test antigen over the chip's capture surface at a range of concentrations, e.g., at 5000 nM, 1000 nM, 200 nM, 40 nM, 8 nM and 2 nM, and at 0 nM (i.e., buffer alone); and
4. Determining the affinity of binding of test antibody to test antigen using SPR at 25° C. Buffer may be at pH 7.6, 150 mM NaCl, 0.05% detergent (e.g., P20) and 3 mM EDTA. Buffer may optionally contain 10 mM HEPES. HBS-EP can be used as running buffer. HBS-EP is available from Teknova Inc (California; catalogue number H8022).

Regeneration of the capture surface can be carried out with 10 mM glycine at pH 1.7. This removes the captured antibody and allows the surface to be used for another interaction. The binding data can be fitted to 1:1 model inherent using standard techniques, e.g., using a model inherent to the ProteOn XPR36™ analysis software.

A variety of SPR instruments are known, such as Biacore™, ProteOn XPR36™ (Bio-Rad®), and KinExA® (Sapidyne Instruments, Inc). Worked examples of SPR are found in Example 7 of WO2018/029474.

As described, affinity may be determined by SPR with the antibody in Fab format, with the antigen coupled to the chip surface and the test antibody passed over the chip in Fab format in solution, to determine affinity of the monomeric antibody-antigen interaction. Affinity can be determined at any desired pH, e.g., pH 5.5 or pH 7.6, and any desired temperature e.g., 25° C. or 37° C. As reported in Example 7 of WO2018/029474, antibodies according to the present invention bound human ICOS with an apparent affinity of less than 2 nM, as determined by SPR using the antibody in monovalent (Fab) format.

Other ways to measure binding of an antibody to ICOS include fluorescence activated cell sorting (FACS), e.g., using cells (e.g., CHO cells) with exogenous surface expression of ICOS or activated primary T cells expressing endogenous levels of ICOS. Antibody binding to ICOS-expressing cells as measured by FACS indicates that the antibody is able to bind the extracellular (EC) domain of ICOS.

### ICOS Receptor Agonism

The ICOS ligand (ICOSL, also known as B7-H2) is a cell surface expressed molecule that binds to the ICOS receptor [17]. This intercellular ligand-receptor interaction promotes multimerisation of ICOS on the T cell surface, activating the receptor and stimulating downstream signalling in the T cell. In effector T cells, this receptor activation stimulates the effector T cell response.

Anti-ICOS antibodies may act as agonists of ICOS, mimicking and even surpassing this stimulatory effect of the native ICOS ligand on the receptor. Such agonism may result from ability of the antibody to promote multimerisation of ICOS on the T cell. One mechanism for this is where the antibodies form intercellular bridges between ICOS on the T cell surface and receptors on an adjacent cell (e.g., B cell, antigen-presenting cell, or other immune cell), such as Fc receptors. Another mechanism is where antibodies having multiple (e.g., two) antigen-binding sites (e.g., two VH-VL domain pairs) bridge multiple ICOS receptor molecules and so promote multimerisation. A combination of these mechanisms may occur.

Agonism can be tested for in in vitro T cell activation assays, using antibody in soluble form (e.g., in immunoglobulin format or other antibody format comprising two spatially separated antigen-binding sites, e.g., two VH-VL pairs), either including or excluding a cross-linking agent, or using antibody bound to a solid surface to provide a tethered array of antigen-binding sites. Agonism assays may use a human ICOS positive T lymphocyte cell line such as MJ cells (ATCC CRL-8294) as the target T cell for activation in such assays. One or more measures of T cell activation can be determined for a test antibody and compared with a reference molecule or a negative control to determine whether there is a statistically significant (p<0.05) difference in T cell activation effected by the test antibody compared with the reference molecule or the control. One suitable measure of T cell activation is production of cytokines, e.g., IFNγ, TNFα or IL-2. The skilled person will include suitable controls as appropriate, standardising assay conditions between test antibody and control. A suitable negative control is an antibody in the same format (e.g., isotype control) that does not bind ICOS, e.g., an antibody specific for an antigen that is not present in the assay system. A significant difference is observed for test antibody relative to a cognate isotype control within the dynamic range of the assay is indicative that the antibody acts as an agonist of the ICOS receptor in that assay.

An agonist antibody may be defined as one which, when tested in a T cell activation assay:
has a significantly lower EC50 for induction of IFNγ production compared with control antibody;
induces significantly higher maximal IFNγ production compared with control antibody;
has a significantly lower EC50 for induction of IFNγ production compared with ICOSL-Fc;
induces significantly higher maximal IFNγ production compared with ICOSL-Fc;
has a significantly lower EC50 for induction of IFNγ production compared with reference antibody C398.4A; and/or
induces significantly higher maximal IFNγ production compared with reference antibody C398.4A.

In vitro T cell assays include the bead-bound assay of Example 13 of WO2018/029474, the plate-bound assay of Example 14 of WO2018/029474 and the soluble form assay of Example 15 of WO2018/029474.

A significantly lower or significantly higher value may for example be up to 0.5-fold different, up to 0.75-fold different, up to 2-fold different, up to 3-fold different, up to 4-fold different or up to 5-fold different, compared with the reference or control value.

Thus, in one example, an antibody according to the present invention has a significantly lower, e.g., at least 2-fold lower, EC50 for induction of IFNγ in an MJ cell activation assay using the antibody in bead-bound format, compared with control.

The bead-bound assay uses the antibody (and, for control or reference experiments, the control antibody, reference antibody or ICOSL-Fc) bound to the surface of beads. Magnetic beads may be used, and various kinds are commercially available, e.g., Tosyl-activated DYNABEADS M-450 (DYNAL Inc, 5 Delaware Drive, Lake Success, N.Y. 11042 Prod No. 140.03, 140.04). Beads may be coated as described in Example 13 of WO2018/029474, or generally by dissolving the coating material in carbonate buffer (pH 9.6, 0.2 M) or other method known in the art. Use of beads conveniently allows the quantity of protein bound to the bead surface to be determined with a good degree of accuracy. Standard Fc-protein quantification methods can be used for coupled protein quantification on beads. Any suitable method can be used, with reference to a relevant standard within the dynamic range of the assay. DELFIA is exemplified in Example 13 of WO2018/029474, but ELISA or other methods could be used.

Agonism activity of an antibody can also be measured in primary human T lymphocytes ex vivo. The ability of an antibody to induce expression of IFNγ in such T cells is indicative of ICOS agonism. Described herein are two T cell activation assays using primary cells—see Example 2 of WO2018/029474, T cell activation assay 1 and T cell activation assay 2. Preferably, an antibody will show significant (p<0.05) induction of IFNγ at 5 μg/ml compared with control antibody in T cell activation assay 1 and/or T cell activation assay 2. As noted above, an anti-ICOS antibody may stimulate T cell activation to a greater degree than ICOS-L or C398.4 in such an assay. Thus, the antibody may show significantly (p<0.05) greater induction of IFNγ at 5 μg/ml compared with the control or reference antibody in T cell activation assay 1 or 2. TNFα or IL-2 induction may be measured as an alternative assay readout.

Agonism of an anti-ICOS antibody may contribute to its ability to change the balance between populations of TReg and TEff cells in vivo, e.g., in a site of pathology such as a tumour microenvironment, in favour of TEff cells. The ability of an antibody to enhance tumour cell killing by activated ICOS-positive effector T cells may be determined, as discussed elsewhere herein.

### PD-L1 or PD-1 Receptor Antagonism

PD-L1 or PD-1 inhibitors may act as PD-L1 or PD-1 antagonist. That is, they inhibit the binding of PD-L1 to PD-1 (or the binding of PD-1 to PD-L1).

### T Cell Dependent Killing

Effector T cell function can be determined in a biologically relevant context using an in vitro co-culture assay where tumour cells are incubated with relevant immune cells to trigger immune cell-dependent killing, in which the effect of an anti-ICOS antibody on tumour cell killing by TEffs is observed.

The ability of an antibody to enhance tumour cell killing by activated ICOS-positive effector T cells may be determined. An anti-ICOS antibody may stimulate significantly greater (p<0.05) tumour cell killing compared with a control antibody. An anti-ICOS antibody may stimulate similar or greater tumour cell killing in such an assay as compared with a reference molecule such as the ICOS ligand or the C398.4 antibody. A similar degree of tumour cell killing can be represented as the assay readout for the test antibody being less than two-fold different from that for the reference molecule.

### ICOS Ligand-Receptor Neutralisation Potency

An antibody used in the present invention may be one which inhibits binding of ICOS to its ligand ICOSL.

The degree to which an antibody inhibits binding of the ICOS receptor to its ligand is referred to as its ligand-receptor neutralising potency. Potency is normally expressed as an IC50 value, in pM unless otherwise stated. In ligand-binding studies, IC50 is the concentration that reduces receptor binding by 50% of maximal specific binding level. IC50 may be calculated by plotting % specific receptor binding as a function of the log of the antibody concentration, and using a software program such as Prism (GraphPad) to fit a sigmoidal function to the data to generate IC50 values. Neutralising potency may be determined in an HTRF assay. A detailed working example of an HTRF assay for ligand-receptor neutralising potency is set out in Example 8 of WO2018/029474.

An IC50 value may represent the mean of a plurality of measurements. Thus, for example, IC50 values may be obtained from the results of triplicate experiments, and a mean IC50 value can then be calculated.

An antibody may have an IC50 of 1 mM or less in a ligand-receptor neutralisation assay, e.g., 0.5 mM or less. The IC50 may be, 30 nM or less, 20 nM or less, 10 nM or less, 5 nM or less, 4 nM or less, 3 nM or less or 2 nM or less. The IC50 may be at least 0.1 nM, at least 0.5 nM or at least 1 nM.

### Antibodies

As described in more detail in the Examples of WO2018/029474, we isolated and characterised antibodies of particular interest, designated STIM001, STIM002, STIM002-B, STIM003, STIM004, STIM005, STIM006, STIM007, STIM008 and STIM009. In various aspects of the invention, unless context dictates otherwise, antibodies may be selected from any of these antibodies, or from the sub-set of STIM001, STIM002, STIM003, STIM004 and STIM005. Sequences of each of these antibodies are provided in the appended sequence listing, wherein for each antibody the following sequences are shown: nucleotide sequence encoding VH domain; amino acid sequence of VH domain; VH CDR1 amino acid sequence, VH CDR2 amino acid sequence; VH CDR3 amino acid sequence; nucleotide sequence encoding VL domain; amino acid sequence of VL domain; VL CDR1 amino acid sequence; VL CDR2 amino acid sequence; and VL CDR3 amino acid sequence, respectively. The present invention encompasses anti-ICOS antibodies having the VH and/or VL domain sequences of all antibodies shown in the appended sequence listing and/or in the drawings, as well as antibodies comprising the HCDRs and/or LCDRs of those antibodies, and optionally having the full heavy chain and/or full light chain amino acid sequence.

STIM001 has a heavy chain variable region (V_{H}) amino acid sequence of Seq ID No:366, comprising the CDRH1 amino acid sequence of Seq ID No:363, the CDRH2 amino acid sequence of Seq ID No:364, and the CDRH3 amino acid sequence of Seq ID No:365. The heavy chain nucleic acid sequence of the V_{H} domain is Seq ID No:367. STIM001 has a light chain variable region (V_{L}) amino acid sequence of Seq ID No:373, comprising the CDRL1 amino acid sequence of Seq ID No:370, the CDRL2 amino acid sequence of Seq ID No:371, and the CDRL3 amino acid sequence of Seq ID No:372. The light chain nucleic acid sequence of the V_{L} domain is Seq ID No:374. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:368 (heavy chain nucleic acid sequence Seq ID No:369). A full length light chain amino acid sequence is Seq ID No:375 (light chain nucleic acid sequence Seq ID No:376).

STIM002 has a heavy chain variable region (V_{H}) amino acid sequence of Seq ID No:380, comprising the CDRH1 amino acid sequence of Seq ID No:377, the CDRH2 amino acid sequence of Seq ID No:378, and the CDRH3 amino acid sequence of Seq ID No:379. The heavy chain nucleic acid sequence of the V_{H} domain is Seq ID No:381. STIM002 has a light chain variable region (V_{L}) amino acid sequence of Seq ID No:387, comprising the CDRL1 amino acid sequence of Seq ID No:384, the CDRL2 amino acid sequence of Seq ID No:385, and the CDRL3 amino acid sequence of Seq ID No:386. The light chain nucleic acid sequence of the V_{L} domain is Seq ID No:388 or Seq ID No:519. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:382 (heavy chain nucleic acid sequence Seq ID No:383). A full length light chain amino acid sequence is Seq ID No:389 (light chain nucleic acid sequence Seq ID No:390 or Seq ID NO:520).

STIM002-B has a heavy chain variable region (V_{H}) amino acid sequence of Seq ID No:394, comprising the CDRH1 amino acid sequence of Seq ID No:391, the CDRH2 amino acid sequence of Seq ID No:392, and the CDRH3 amino acid sequence of Seq ID No:393. The heavy chain nucleic acid sequence of the V_{H} domain is Seq ID No:395. STIM002-B has a light chain variable region (V_{L}) amino acid sequence of Seq ID No:401, comprising the CDRL1 amino acid sequence of Seq ID No:398, the CDRL2 amino acid sequence of Seq ID No:399, and the CDRL3 amino acid sequence of Seq ID No:400. The light chain nucleic acid sequence of the V_{L} domain is Seq ID No:402. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:396 (heavy chain nucleic acid sequence Seq ID No:397). A full length light chain amino acid sequence is Seq ID No:403 (light chain nucleic acid sequence Seq ID No:404).

STIM003 has a heavy chain variable region (V_{H}) amino acid sequence of Seq ID No:408, comprising the CDRH1 amino acid sequence of Seq ID No:405, the CDRH2 amino acid sequence of Seq ID No:406, and the CDRH3 amino acid sequence of Seq ID No:407. The heavy chain nucleic acid sequence of the V_{H} domain is Seq ID No:409 or Seq ID No:521. STIM003 has a light chain variable region (V_{L}) amino acid sequence of Seq ID No:415, comprising the CDRL1 amino acid sequence of Seq ID No:412, the CDRL2 amino acid sequence of Seq ID No:413, and the CDRL3 amino acid sequence of Seq ID No:414. The light chain nucleic acid sequence of the V_{L} domain is Seq ID No:4416. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:410 (heavy chain nucleic acid sequence Seq ID No:411 or Seq ID No:522). A full length light chain amino acid sequence is Seq ID No:417 (light chain nucleic acid sequence Seq ID No:418).

STIM004 has a heavy chain variable region (V_{H}) amino acid sequence of Seq ID No:422, comprising the CDRH1 amino acid sequence of Seq ID No:419, the CDRH2 amino acid sequence of Seq ID No:420, and the CDRH3 amino acid sequence of Seq ID No:421. The heavy chain nucleic acid sequence of the V_{H} domain is Seq ID No:423. STIM004 has a light chain variable region (V_{L}) amino acid sequence of Seq ID No:429, comprising the CDRL1 amino acid sequence of Seq ID No:426, the CDRL2 amino acid sequence of Seq ID No:427, and the CDRL3 amino acid sequence of Seq ID No:428. The light chain nucleic acid sequence of the V_{L} domain is Seq ID No:430 or Seq ID No:431. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:424 (heavy chain nucleic acid sequence Seq ID No:425). A full length light chain amino acid sequence is Seq ID No:432 (light chain nucleic acid sequence Seq ID No:433 or Seq ID no: 434).

STIM005 has a heavy chain variable region (V_{H}) amino acid sequence of Seq ID No:438, comprising the CDRH1 amino acid sequence of Seq ID No:435, the CDRH2 amino acid sequence of Seq ID No:436, and the CDRH3 amino acid sequence of Seq ID No:437. The heavy chain nucleic acid sequence of the V_{H} domain is Seq ID No:439. STIM005 has a light chain variable region (V_{L}) amino acid sequence of Seq ID No:445, comprising the CDRL1 amino acid sequence of Seq ID No:442, the CDRL2 amino acid sequence of Seq ID No:443, and the CDRL3 amino acid sequence of Seq ID No:444. The light chain nucleic acid sequence of the V_{L} domain is Seq ID No:446. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:440 (heavy chain nucleic acid sequence Seq ID No:441). A full length light chain amino acid sequence is Seq ID No:447 (light chain nucleic acid sequence Seq ID No:448).

STIM006 has a heavy chain variable region (V_{H}) amino acid sequence of Seq ID No:452, comprising the CDRH1 amino acid sequence of Seq ID No:449, the CDRH2 amino acid sequence of Seq ID No:450, and the CDRH3 amino acid sequence of Seq ID No:451. The heavy chain nucleic acid sequence of the V_{H} domain is Seq ID No:453. STIM006 has a light chain variable region (V_{L}) amino acid sequence of Seq ID No:459, comprising the CDRL1 amino acid sequence of Seq ID No:456, the CDRL2 amino acid sequence of Seq ID No:457, and the CDRL3 amino acid sequence of Seq ID No:458. The light chain nucleic acid sequence of the V_{L} domain is Seq ID No:460. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:454 (heavy chain nucleic acid sequence Seq ID No:455). A full length light chain amino acid sequence is Seq ID No:461 (light chain nucleic acid sequence Seq ID No:462).

STIM007 has a heavy chain variable region (V_{H}) amino acid sequence of Seq ID No:466, comprising the CDRH1 amino acid sequence of Seq ID No:463, the CDRH2 amino acid sequence of Seq ID No:464, and the CDRH3 amino acid sequence of Seq ID No:465. The heavy chain nucleic acid sequence of the V_{H} domain is Seq ID No:467. STIM007 has a light chain variable region (V_{L}) amino acid sequence of Seq ID No:473, comprising the CDRL1 amino acid sequence of Seq ID No:470, the CDRL2 amino acid sequence of Seq ID No:471, and the CDRL3 amino acid sequence of Seq ID No:472. The light chain nucleic acid sequence of the V_{L} domain is Seq ID No:474. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:468 (heavy chain nucleic acid sequence Seq ID No:469). A full length light chain amino acid sequence is Seq ID No:475 (light chain nucleic acid sequence Seq ID No:476).

STIM008 has a heavy chain variable region (V_{H}) amino acid sequence of Seq ID No:480, comprising the CDRH1 amino acid sequence of Seq ID No:477, the CDRH2 amino acid sequence of Seq ID No:478, and the CDRH3 amino acid sequence of Seq ID No:479. The heavy chain nucleic acid sequence of the V_{H} domain is Seq ID No:481. STIM008 has a light chain variable region (V_{L}) amino acid sequence of Seq ID No:487, comprising the CDRL1 amino acid sequence of Seq ID No:484, the CDRL2 amino acid sequence of Seq ID No:485, and the CDRL3 amino acid sequence of Seq ID No:486. The light chain nucleic acid sequence of the V_{L} domain is Seq ID No:488. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:482 (heavy chain nucleic acid sequence Seq ID No:483). A full length light chain amino acid sequence is Seq ID No:489 (light chain nucleic acid sequence Seq ID No:490).

STIM009 has a heavy chain variable region (V_{H}) amino acid sequence of Seq ID No:494, comprising the CDRH1 amino acid sequence of Seq ID No:491, the CDRH2 amino acid sequence of Seq ID No:492, and the CDRH3 amino acid sequence of Seq ID No:493. The heavy chain nucleic acid sequence of the V_{H} domain is Seq ID No:495. STIM009 has a light chain variable region (V_{L}) amino acid sequence of Seq ID No:501, comprising the CDRL1 amino acid sequence of Seq ID No:498, the CDRL2 amino acid sequence of Seq ID No:499, and the CDRL3 amino acid sequence of Seq ID No:500. The light chain nucleic acid sequence of the V_{L} domain is Seq ID No:502. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:496 (heavy chain nucleic acid sequence Seq ID No:497). A full length light chain amino acid sequence is Seq ID No:503 (light chain nucleic acid sequence Seq ID No:504).

Antibodies according to the present invention are immunoglobulins or molecules comprising immunoglobulin domains, whether natural or partly or wholly synthetically produced. Antibodies may be IgG, IgM, IgA, IgD or IgE molecules or antigen-specific antibody fragments thereof (including, but not limited to, a Fab, F(ab′)2, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria. Antibodies can be humanised using routine technology. The term antibody covers any polypeptide or protein comprising an antibody antigen-binding site. An antigen-binding site (paratope) is the part of an antibody that binds to and is complementary to the epitope of its target antigen (ICOS).

The term “epitope” refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The antigen binding site is a polypeptide or domain that comprises one or more CDRs of an antibody and is capable of binding the antigen. For example, the polypeptide comprises a CDR3 (e.g., HCDR3). For example the polypeptide comprises CDRs 1 and 2 (e.g., HCDR1 and 2) or CDRs 1-3 of a variable domain of an antibody (e.g., HCDRs1-3).

An antibody antigen-binding site may be provided by one or more antibody variable domains. In an example, the antibody binding site is provided by a single variable domain, e.g., a heavy chain variable domain (VH domain) or a light chain variable domain (VL domain). In another example, the binding site comprises a VH/VL pair or two or more of such pairs. Thus, an antibody antigen-binding site may comprise a VH and a VL.

The antibody may be a whole immunoglobulin, including constant regions, or may be an antibody fragment. An antibody fragment is a portion of an intact antibody, for example comprising the antigen binding and/or variable region of the intact antibody. Examples of antibody fragments include:
(i) a Fab fragment, a monovalent fragment consisting of the VL, VH, CL and CH1 domains; (ii) a F(ab′)2 fragment, a bivalent fragment including two Fab fragments linked by a disulfide bridge at the hinge region;
(iii) an Fd fragment consisting of the VH and CH1 domains;
(iv) an Fv fragment consisting of the VL and VH domains of a single arm of an antibody,
(v) a dAb fragment (Ward et al., (1989) Nature 341:544-546; which is incorporated by reference herein in its entirety), which consists of a VH or VL domain; and
(vi) an isolated complementarity determining region (CDR) that retains specific antigen-binding functionality.

Further examples of antibodies are H2 antibodies that comprise a dimer of a heavy chain (5′-VH-(optional hinge)-CH2-CH3-3′) and are devoid of a light chain.

Single-chain antibodies (e.g., scFv) are a commonly used fragment. Multispecific antibodies may be formed from antibody fragments. An antibody of the invention may employ any such format, as appropriate.

Optionally, the antibody immunoglobulin domains may be fused or conjugated to additional polypeptide sequences and/or to labels, tags, toxins or other molecules. Antibody immunoglobulin domains may be fused or conjugated to one or more different antigen binding regions, providing a molecule that is able to bind a second antigen in addition to ICOS. An antibody of the present invention may be a multispecific antibody, e.g., a bispecific antibody, comprising (i) an antibody antigen binding site for ICOS and (ii) a further antigen binding site (optionally an antibody antigen binding site, as described herein) which recognises another antigen (e.g., PD-L1).

An antibody normally comprises an antibody VH and/or VL domain. Isolated VH and VL domains of antibodies are also part of the invention. The antibody variable domains are the portions of the light and heavy chains of antibodies that include amino acid sequences of complementarity determining regions (CDRs; ie., CDR1, CDR2, and CDR3), and framework regions (FRs). Thus, within each of the VH and VL domains are CDRs and FRs. A VH domain comprises a set of HCDRs, and a VL domain comprises a set of LCDRs. VH refers to the variable domain of the heavy chain. VL refers to the variable domain of the light chain. Each VH and VL is typically composed of three CDRs and four FRs, arranged from amino-terminus to carboxy-terminus in the following order: FR1, CDR1, FR2, CDR2, FR3, CDR3, FR4. According to the methods used in this invention, the amino acid positions assigned to CDRs and FRs may be defined according to Kabat (Sequences of Proteins of Immunological Interest (National Institutes of Health, Bethesda, Md., 1987 and 1991)) or according to IMGT nomenclature. An antibody may comprise an antibody VH domain comprising a VH CDR1, CDR2 and CDR3 and a framework. It may alternatively or also comprise an antibody VL domain comprising a VL CDR1, CDR2 and CDR3 and a framework. Examples of antibody VH and VL domains and CDRs according to the present invention are as listed in the appended sequence listing that forms part of the present disclosure. The CDRs shown in the sequence listing are defined according to the IMGT system [18]. All VH and VL sequences, CDR sequences, sets of CDRs and sets of HCDRs and sets of LCDRs disclosed herein represent aspects and embodiments of the invention. As described herein, a “set of CDRs” comprises CDR1, CDR2 and CDR3. Thus, a set of HCDRs refers to HCDR1, HCDR2 and HCDR3, and a set of LCDRs refers to LCDR1, LCDR2 and LCDR3. Unless otherwise stated, a “set of CDRs” includes HCDRs and LCDRs.

An antibody the invention may comprise one or more CDRs as described herein, e.g. a CDR3, and optionally also a CDR1 and CDR2 to form a set of CDRs. The CDR or set of CDRs may be a CDR or set of CDRs of any of STIM001, STIM002, STIM002-B, STIM003, STIM004, STIM005, STIM006, STIM007, STIM008 and STIM009, or may be a variant thereof as described herein.

The invention provides antibodies comprising an HCDR1, HCDR2 and/or HCDR3 of any of antibodies STIM001, STIM002, STIM002-B, STIM003, STIM004, STIM005, STIM006, STIM007, STIM008 and STIM009 and/or an LCDR1, LCDR2 and/or LCDR3 of any of these antibodies, e.g. a set of CDRs. The antibody may comprise a set of VH CDRs of one of these antibodies. Optionally it may also comprise a set of VL CDRs of one of these antibodies, and the VL CDRs may be from the same or a different antibody as the VH CDRs.

A VH domain comprising a disclosed set of HCDRs, and/or a VL domain comprising a disclosed set of LCDRs, are also provided by the invention.

Typically, a VH domain is paired with a VL domain to provide an antibody antigen-binding site, although as discussed further below a VH or VL domain alone may be used to bind antigen. The STIM003 VH domain may be paired with the STIM003 VL domain, so that an antibody antigen-binding site is formed comprising both the STIM003 VH and VL domains. Analogous embodiments are provided for the other VH and VL domains disclosed herein. In other embodiments, the STIM003 VH is paired with a VL domain other than the STIM003 VL. Light-chain promiscuity is well established in the art. Again, analogous embodiments are provided by the invention for the other VH and VL domains disclosed herein.

Thus, the VH of any of antibodies STIM001, STIM002, STIM003, STIM004 and STIM005 may be paired with the VL of any of antibodies STIM001, STIM002, STIM003, STIM004 and STIM005. Further, the VH of any of antibodies STIM001, STIM002, STIM002-B, STIM003, STIM004, STIM005, STIM006, STIM007, STIM008 and STIM009 may be paired with the VL of any of antibodies STIM001, STIM002, STIM002-B, STIM003, STIM004, STIM005, STIM006, STIM007, STIM008 or STIM009.

An antibody may comprise one or more CDRs, e.g. a set of CDRs, within an antibody framework. The framework regions may be of human germline gene segment sequences. Thus, the antibody may be a human antibody having a VH domain comprising a set of HCDRs in a human germline framework. Normally the antibody also has a VL domain comprising a set of LCDRs, e.g. in a human germline framework. An antibody “gene segment”, e.g., a VH gene segment, D gene segment, or JH gene segment refers to oligonucleotide having a nucleic acid sequence from which that portion of an antibody is derived, e.g., a VH gene segment is an oligonucleotide comprising a nucleic acid sequence that corresponds to a polypeptide VH domain from FR1 to part of CDR3. Human V, D and J gene segments recombine to generate the VH domain, and human V and J segments recombine to generate the VL domain. The D domain or region refers to the diversity domain or region of an antibody chain. J domain or region refers to the joining domain or region of an antibody chain. Somatic hypermutation may result in an antibody VH or VL domain having framework regions that do not exactly match or align with the corresponding gene segments, but sequence alignment can be used to identify the closest gene segments and thus identify from which particular combination of gene segments a particular VH or VL domain is derived. When aligning antibody sequences with gene segments, the antibody amino acid sequence may be aligned with the amino acid sequence encoded by the gene segment, or the antibody nucleotide sequence may be aligned directly with the nucleotide sequence of the gene segment.

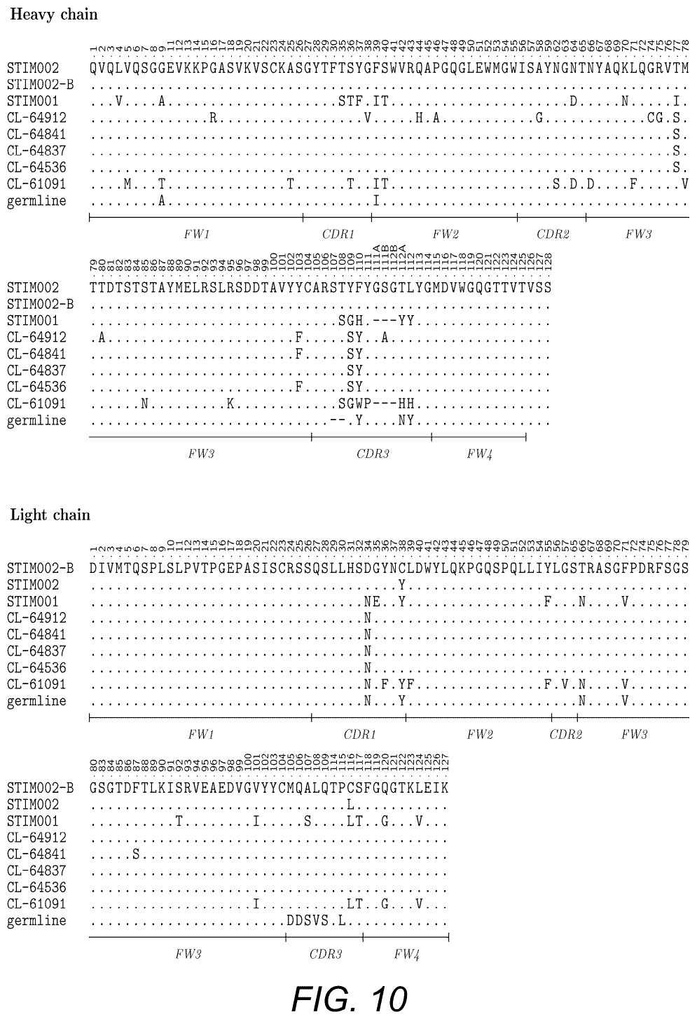

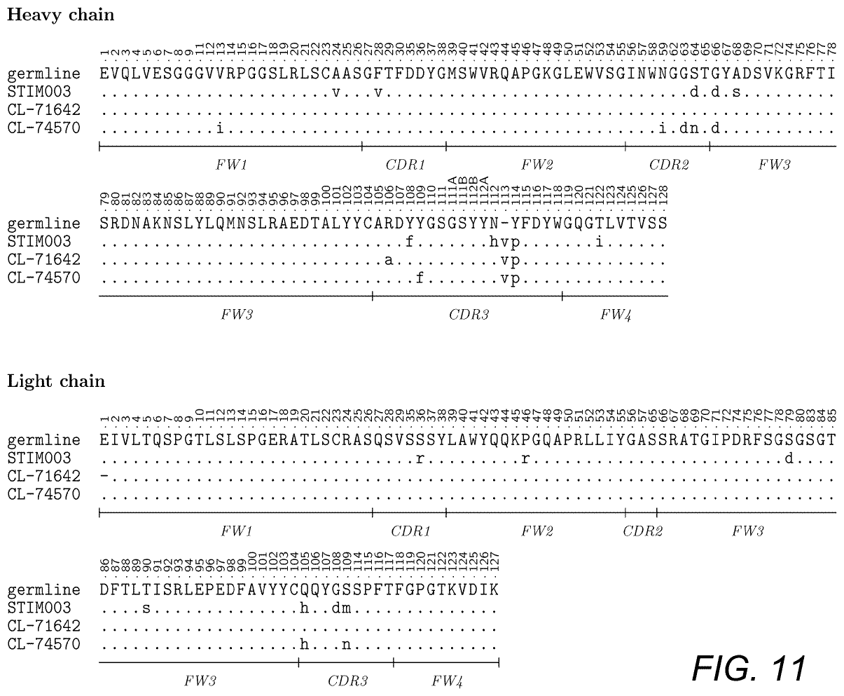

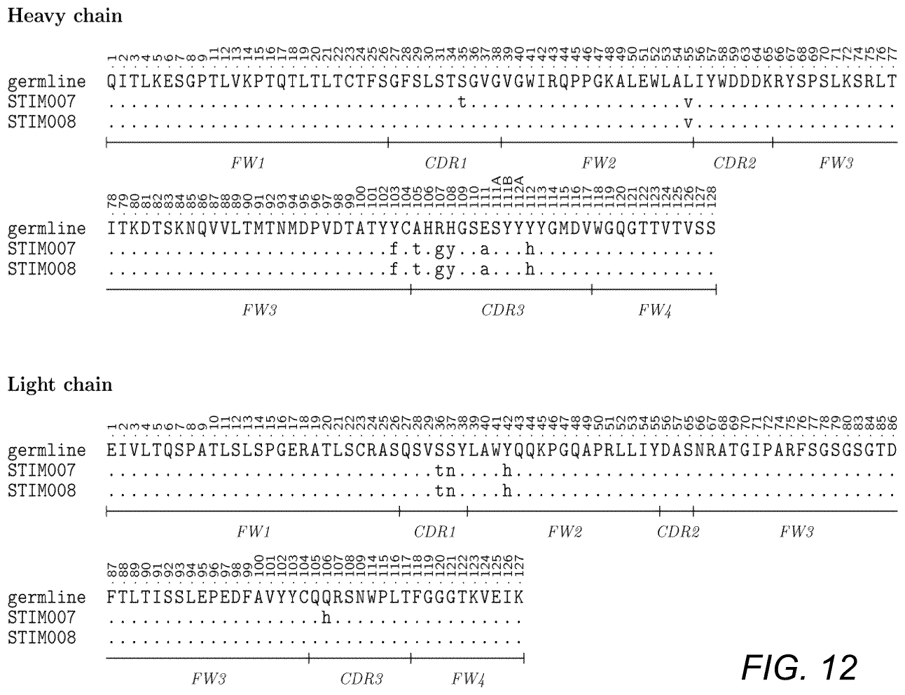

Alignments of STIM antibody VH and VL domain sequences against related antibodies and against human germline sequences are shown in FIG. 10, FIG. 11 and FIG. 12.

An antibody of the invention may be a human antibody or a chimaeric antibody comprising human variable regions and non-human (e.g., mouse) constant regions. The antibody of the invention for example has human variable regions, and optionally also has human constant regions.

Thus, antibodies optionally include constant regions or parts thereof, e.g., human antibody constant regions or parts thereof. For example, a VL domain may be attached at its C-terminal end to antibody light chain kappa or lambda constant domains. Similarly, an antibody VH domain may be attached at its C-terminal end to all or part (e.g. a CH1 domain or Fc region) of an immunoglobulin heavy chain constant region derived from any antibody isotype, e.g. IgG, IgA, IgE and IgM and any of the isotype sub-classes, such as IgG1 or IgG4.

Examples of human heavy chain constant regions are shown in Table S1.

Constant regions of antibodies of the invention may alternatively be non-human constant regions. For example, when antibodies are generated in transgenic animals (examples of which are described elsewhere herein), chimaeric antibodies may be produced comprising human variable regions and non-human (host animal) constant regions. Some transgenic animals generate fully human antibodies. Others have been engineered to generate antibodies comprising chimaeric heavy chains and fully human light chains. Where antibodies comprise one or more non-human constant regions, these may be replaced with human constant regions to provide antibodies more suitable for administration to humans as therapeutic compositions, as their immunogenicity is thereby reduced.

Digestion of antibodies with the enzyme papain, results in two identical antigen-binding fragments, known also as “Fab” fragments, and a “Fc” fragment, having no antigen-binding activity but having the ability to crystallize. “Fab” when used herein refers to a fragment of an antibody that includes one constant and one variable domain of each of the heavy and light chains. The term “Fc region” herein is used to define a C-terminal region of an immunoglobulin heavy chain, including native-sequence Fc regions and variant Fc regions. The “Fc fragment” refers to the carboxy-terminal portions of both H chains held together by disulfides. The effector functions of antibodies are determined by sequences in the Fc region, the region which is also recognised by Fc receptors (FcR) found on certain types of cells. Digestion of antibodies with the enzyme pepsin, results in the a F(ab′)2 fragment in which the two arms of the antibody molecule remain linked and comprise two-antigen binding sites. The F(ab′)2 fragment has the ability to crosslink antigen.

“Fv” when used herein refers to the minimum fragment of an antibody that retains both antigen-recognition and antigen-binding sites. This region consists of a dimer of one heavy and one light chain variable domain in tight, non-covalent or covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognise and bind antigen, although at a lower affinity than the entire binding site.

Antibodies disclosed herein may be modified to increase or decrease serum half-life. In one embodiment, one or more of the following mutations: T252L, T254S or T256F are introduced to increase biological half-life of the antibody. Biological half-life can also be increased by altering the heavy chain constant region CH, domain or CL region to contain a salvage receptor binding epitope taken from two loops of a CH₂ domain of an Fc region of an IgG, as described in U.S. Pat. Nos. 5,869,046 and 6,121,022, the modifications described therein are incorporated herein by reference. In another embodiment, the Fc hinge region of an antibody or antigen-binding fragment of the invention is mutated to decrease the biological half-life of the antibody or fragment. One or more amino acid mutations are introduced into the CH₂—CH₃ domain interface region of the Fc-hinge fragment such that the antibody or fragment has impaired Staphylococcal protein A (SpA) binding relative to native Fc-hinge domain SpA binding. Other methods of increasing serum half-life are known to those skilled in the art. Thus, in one embodiment, the antibody or fragment is PEGylated. In another embodiment, the antibody or fragment is fused to an albumin-biding domain, e.g. an albumin binding single domain antibody (dAb). In another embodiment, the antibody or fragment is PASylated (i.e. genetic fusion of polypeptide sequences composed of PAS (XL-Protein GmbH) which forms uncharged random coil structures with large hydrodynamic volume). In another embodiment, the antibody or fragment is XTENylated®/rPEGylated (i.e. genetic fusion of non-exact repeat peptide sequence (Amunix, Versartis) to the therapeutic peptide). In another embodiment, the antibody or fragment is ELPylated (i.e. genetic fusion to ELP repeat sequence (PhaseBio)). These various half-life extending fusions are described in more detail in Strohl, BioDrugs (2015) 29:215-239, which fusions, e.g. in Tables 2 and 6, are incorporated herein by reference.

The antibody may have a modified constant region which increases stability. Thus, in one embodiment, the heavy chain constant region comprises a Ser228Pro mutation. In another embodiment, the antibodies and fragments disclosed herein comprise a heavy chain hinge region that has been modified to alter the number of cysteine residues. This modification can be used to facilitate assembly of the light and heavy chains or to increase or decrease the stability of the antibody.

The details above may apply to any ICOS modulators or PD-L1 inhibitors that are antibodies.

### Fc Effector Functions, ADCC, ADCP and CDC

As discussed above, anti-ICOS antibodies can be provided in various isotypes and with different constant regions. Examples of human IgG antibody heavy chain constant region sequences are shown in Table S1. The Fc region of the antibody primarily determines its effector function in terms of Fc binding, antibody-dependent cell-mediated cytotoxicity (ADCC) activity, complement dependent cytotoxicity (CDC) activity and antibody-dependent cell phagocytosis (ADCP) activity. These “cellular effector functions”, as distinct from effector T cell function, involve recruitment of cells bearing Fc receptors to the site of the target cells, resulting in killing of the antibody-bound cell. In addition to ADCC and CDC, the ADCP mechanism [19] represents a means of depleting antibody-bound T cells, and thus targeting high ICOS expressing TRegs for deletion.

Cellular effector functions ADCC, ADCP and/or CDC may also be exhibited by antibodies lacking Fc regions. Antibodies may comprise multiple different antigen-binding sites, one directed to ICOS and another directed to a target molecule where engagement of that target molecule induces ADCC, ADCP and/or CDC, e.g., an antibody comprising two scFv regions joined by a linker, where one scFv can engage an effector cell.

An antibody according to the present invention may be one that exhibits ADCC, ADCP and/or CDC. Alternatively, an antibody according to the present invention may lack ADCC, ADCP and/or CDC activity. In either case, an antibody according to the present invention may comprise, or may optionally lack, an Fc region that binds to one or more types of Fc receptor. Use of different antibody formats, and the presence or absence of FcR binding and cellular effector functions, allow the antibody to be tailored for use in particular therapeutic purposes as discussed elsewhere herein.

A suitable antibody format for some therapeutic applications employs a wild-type human IgG1 constant region. A constant region may be an effector-enabled IgG1 constant region, optionally having ADCC and/or CDC and/or ADCP activity. A suitable wild type human IgG1 constant region sequence is SEQ ID NO: 340 (IGHG1*01). Further examples of human IgG1 constant regions are shown in Table S1.

For testing of candidate therapeutic antibodies in mouse models of human disease, an effector positive mouse constant region, such as mouse IgG2a (mIgG2a), may be included instead of an effector positive human constant region.

A constant region may be engineered for enhanced ADCC and/or CDC and/or ADCP.

The potency of Fc-mediated effects may be enhanced by engineering the Fc domain by various established techniques. Such methods increase the affinity for certain Fc-receptors, thus creating potential diverse profiles of activation enhancement. This can achieved by modification of one or several amino acid residues [20]. Human IgG1 constant regions containing specific mutations or altered glycosylation on residue Asn297 (e.g., N297Q, EU index numbering) have been shown to enhance binding to Fc receptors. Example mutations are one or more of the residues selected from 239, 332 and 330 for human IgG1 constant regions (or the equivalent positions in other IgG isotypes). An antibody may thus comprise a human IgG1 constant region having one or more mutations independently selected from N297Q, S239D, 1332E and A330L (EU index numbering). A triple mutation (M252Y/S254T/T256E) may be used to enhance binding to FcRn, and other mutations affecting FcRn binding are discussed in Table 2 of [21], any of which may be employed in the present invention.

Increased affinity for Fc receptors can also be achieved by altering the natural glycosylation profile of the Fc domain by, for example, generating under fucosylated or defucosylated variants [22]. Non-fucosylated antibodies harbour a tri-mannosyl core structure of complex-type N-glycans of Fc without fucose residue. These glycoengineered antibodies that lack core fucose residue from the Fc N-glycans may exhibit stronger ADCC than fucosylated equivalents due to enhancement of FcγRIIIa binding capacity. For example, to increase ADCC, residues in the hinge region can be altered to increase binding to Fc-gamma RIII [23]. Thus, an antibody may comprise a human IgG heavy chain constant region that is a variant of a wild-type human IgG heavy chain constant region, wherein the variant human IgG heavy chain constant region binds to human Fcγ receptors selected from the group consisting of FcγRIIB and FcγRIIA with higher affinity than the wild type human IgG heavy chain constant region binds to the human Fcγ receptors. The antibody may comprise a human IgG heavy chain constant region that is a variant of a wild type human IgG heavy chain constant region, wherein the variant human IgG heavy chain constant region binds to human FcγRIIB with higher affinity than the wild type human IgG heavy chain constant region binds to human FcγRIIB. The variant human IgG heavy chain constant region can be a variant human IgG1, a variant human IgG2, or a variant human IgG4 heavy chain constant region. In one embodiment, the variant human IgG heavy chain constant region comprises one or more amino acid mutations selected from G236D, P238D, S239D, S267E, L328F, and L328E (EU index numbering system). In another embodiment, the variant human IgG heavy chain constant region comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F (EU index numbering system). The enhancement of CDC may be achieved by amino acid changes that increase affinity for C1q, the first component of the classic complement activation cascade [24]. Another approach is to create a chimeric Fc domain created from human IgG1 and human IgG3 segments that exploit the higher affinity of IgG3 for C1q [25]. Antibodies of the present invention may comprise mutated amino acids at residues 329, 331 and/or 322 to alter the C1q binding and/or reduced or abolished CDC activity. In another embodiment, the antibodies or antibody fragments disclosed herein may contain Fc regions with modifications at residues 231 and 239, whereby the amino acids are replaced to alter the ability of the antibody to fix complement. In one embodiment, the antibody or fragment has a constant region comprising one or more mutations selected from E345K, E430G, R344D and D356R, in particular a double mutation comprising R344D and D356R (EU index numbering system).

WO2008/137915 described anti-ICOS antibodies with modified Fc regions having enhanced effector function. The antibodies were reported to mediate enhanced ADCC activity as compared to the level of ADCC activity mediated by a parent antibody comprising the VH and VK domains and a wild type Fc region. Antibodies according to the present invention may employ such variant Fc regions having effector function as described therein.

ADCC activity of an antibody may be determined in an assay as described herein. ADCC activity of an anti-ICOS antibody may be determined in vitro using an ICOS positive T cell line as described in Example 10 of WO2018/029474. ADCC activity of an anti-PD-L1 antibody may be determined in vitro in an ADCC assay using PD-L1 expressing cells.

For certain applications (such as in the context of vaccination) it may be preferred to use antibodies without Fc effector function. Antibodies may be provided without a constant region, or without an Fc region—examples of such antibody formats are described elsewhere herein. Alternatively, an antibody may have a constant region which is effector null. An antibody may have a heavy chain constant region that does not bind Fcγ receptors, for example the constant region may comprise a Leu235Glu mutation (i.e., where the wild type leucine residue is mutated to a glutamic acid residue). Another optional mutation for a heavy chain constant region is Ser228Pro, which increases stability. A heavy chain constant region may be an IgG4 comprising both the Leu235Glu mutation and the Ser228Pro mutation. This “IgG4-PE” heavy chain constant region is effector null.

An alternative effector null human constant region is a disabled IgG1. A disabled IgG1 heavy chain constant region may contain alanine at position 235 and/or 237 (EU index numbering), e.g., it may be a IgG1*01 sequence comprising the L235A and/or G237A mutations (“LAGA”).

A variant human IgG heavy chain constant region may comprise one or more amino acid mutations that reduce the affinity of the IgG for human FcγRIIIA, human FcγRIIA, or human FcγRI. In one embodiment, the FcγRIIB is expressed on a cell selected from the group consisting of macrophages, monocytes, B-cells, dendritic cells, endothelial cells, and activated T-cells. In one embodiment, the variant human IgG heavy chain constant region comprises one or more of the following amino acid mutations G236A, S239D, F243L, T256A, K290A, R292P, S298A, Y300L, V3051, A330L, 1332E, E333A, K334A, A339T, and P396L (EU index numbering system). In one embodiment, the variant human IgG heavy chain constant region comprises a set of amino acid mutations selected from the group consisting of: S239D; T256A; K290A; S298A; 1332E; E333A; K334A; A339T; S239D and 1332E; S239D, A330L, and 1332E; S298A, E333A, and K334A; G236A, S239D, and 1332E; and F243L, R292P, Y300L, V3051, and P396L (EU index numbering system). In one embodiment, the variant human IgG heavy chain constant region comprises a S239D, A330L, or 1332E amino acid mutations (EU index numbering system). In one embodiment, the variant human IgG heavy chain constant region comprises an S239D and 1332E amino acid mutations (EU index numbering system). In one embodiment, the variant human IgG heavy chain constant region is a variant human IgG1 heavy chain constant region comprising the S239D and 1332E amino acid mutations (EU index numbering system). In one embodiment, the antibody or fragment comprises an afucosylated Fc region. In another embodiment, the antibody or fragment thereof is defucosylated. In another embodiment, the antibody or fragment is under fucosylated.

An antibody may have a heavy chain constant region that binds one or more types of Fc receptor but does not induce cellular effector functions, i.e., does not mediate ADCC, CDC or ADCP activity. Such a constant region may be unable to bind the particular Fc receptor(s) responsible for triggering ADCC, CDC or ADCP activity.

### Generating and Modifying Antibodies

Methods for identifying and preparing antibodies are well known. Antibodies may be generated using transgenic mice (eg, the Kymouse™, Velocimouse®, Omnimouse®, Xenomouse®, HuMab Mouse® or MeMo Mouse®), rats (e.g., the Omnirat®), camelids, sharks, rabbits, chickens or other non-human animals immunised with ICOS or a fragment thereof or a synthetic peptide comprising an ICOS sequence motif of interest, followed optionally by humanisation of the constant regions and/or variable regions to produce human or humanised antibodies. In an example, display technologies can be used, such as yeast, phage or ribosome display, as will be apparent to the skilled person. Standard affinity maturation, e.g., using a display technology, can be performed in a further step after isolation of an antibody lead from a transgenic animal, phage display library or other library. Representative examples of suitable technologies are described in US20120093818 (Amgen, Inc), which is incorporated by reference herein in its entirety, eg, the methods set out in paragraphs [0309] to [0346].

Immunisation of an ICOS knock out non-human animal with human ICOS antigen facilitates the generation of antibodies that recognise both human and non-human ICOS. As described herein and illustrated in the Examples, an ICOS knock out mouse can be immunised with cells expressing human ICOS to stimulate production of antibodies to human and mouse ICOS in the mouse, which can be recovered and tested for binding to human ICOS and to mouse ICOS. Cross-reactive antibodies can thus be selected, which may be screened for other desirable properties as described herein. Methods of generating antibodies to an antigen (e.g., a human antigen), through immunisation of animals with the antigen where expression of the endogenous antigen (e.g, endogenous mouse antigen) has been knocked-out in the animal, may be performed in animals capable of generating antibodies comprising human variable domains. The genomes of such animals can be engineered to comprise a human or humanised immunoglobulin locus encoding human variable region gene segments, and optionally an endogenous constant region or a human constant region. Recombination of the human variable region gene segments generates human antibodies, which may have either a non-human or human constant region. Non-human constant regions may subsequently be replaced by human constant regions where the antibody is intended for in vivo use in humans. Such methods and knock-out transgenic animals are described in WO2013/061078.

Generally, a Kymouse™, VELOCIMMUNE® or other mouse or rat (optionally an ICOS knock out mouse or rat, as noted) can be challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimaeric antibodies or the variable domains of the light and heavy chains may be isolated directly from antigen-specific lymphocytes.

Initially, high affinity chimaeric antibodies are isolated having a human variable region and a mouse constant region. The antibodies are characterised and selected for desirable characteristics, including affinity, selectivity, agonism, T-cell dependent killing, neutralising potency, epitope, etc. The mouse constant regions are optionally replaced with a desired human constant region to generate the fully human antibody of the invention, for example wild-type or modified IgG1 or IgG4 (for example, SEQ ID NO: 751, 752, 753 in US2011/0065902 (which is incorporated by reference herein in its entirety). While the constant region selected may vary according to specific use, high affinity antigen-binding and target specificity characteristics reside in the variable region.

Thus, in a further aspect, the present invention provides a transgenic non-human mammal having a genome comprising a human or humanised immunoglobulin locus, wherein the mammal does not express ICOS. The mammal may for instance be a knock-out mouse or rat, or other laboratory animal species. Transgenic mice such as the Kymouse™ contain human heavy and light chain immunoglobulin loci inserted at the corresponding endogenous mouse immunoglobulin loci. A transgenic mammal according to the present invention may be one that contains such targeted insertions, or it may contain human heavy and light chain immunoglobulin loci or immunoglobulin genes that are randomly inserted in its genome, inserted at a locus other than the endogenous Ig locus, or provided on an additional chromosome or chromosomal fragment.

Further aspects of the invention are the use of such non-human mammals for producing antibodies to ICOS, and methods of producing antibodies or antibody heavy and/or light chain variable domains in such mammals.

A method of producing an antibody that binds the extracellular domain of human and non-human ICOS may comprise providing a transgenic non-human mammal having a genome comprising a human or humanised immunoglobulin locus, wherein the mammal does not express ICOS, and
(a) immunising the mammal with human ICOS antigen (e.g., with cells expressing human ICOS or with purified recombinant ICOS protein);
(b) isolating antibodies generated by the mammal;
(c) testing the antibodies for ability to bind human ICOS and non-human ICOS; and
(d) selecting one or more antibodies that binds both human and non-human ICOS.

Testing for ability to bind human ICOS and non-human ICOS may be done using surface plasmon resonance, HTRF, FACS or any other method described herein. Optionally, binding affinities for human and mouse ICOS are determined. The affinity, or fold-difference in affinity, of binding to human ICOS and mouse ICOS may be determined, and antibodies displaying species cross-reactivity may thus be selected (affinity thresholds and fold-differences that may be used as selection criteria are exemplified elsewhere herein). Neutralising potency, or fold difference in neutralising potency, of the antibody for inhibiting human and mouse ICOS ligand binding to the human and mouse ICOS receptor respectively may also or alternatively be determined as a way to screen for cross-reactive antibodies, e.g., in an HTRF assay. Again, possible thresholds and fold-differences that may be used as selection criteria are exemplified elsewhere herein.

The method may comprise testing the antibodies for ability to bind non-human ICOS from the same species or from a different species as the immunised mammal. Thus, where the transgenic mammal is a mouse (e.g., a Kymouse™), antibodies may be tested for ability to bind mouse ICOS. Where the transgenic mammal is a rat, antibodies may be tested for ability to bind rat ICOS. However, it may be equally useful to determine cross-reactivity of an isolated antibody for non-human ICOS of another species. Thus, antibodies generated in goats may be tested for binding to rat or mouse ICOS. Optionally, binding to goat ICOS may be determined instead or additionally.

In other embodiments, the transgenic non-human mammal may be immunised with non-human ICOS, optionally ICOS of the same mammalian species (e.g., an ICOS knock-out mouse may be immunised with mouse ICOS) instead of human ICOS. Affinity of isolated antibodies for binding to human ICOS and non-human ICOS is then determined in the same way, and antibodies that bind both human and non-human ICOS are selected.

Nucleic acid encoding an antibody heavy chain variable domain and/or an antibody light chain variable domain of a selected antibody may be isolated. Such nucleic acid may encode the full antibody heavy chain and/or light chain, or the variable domain(s) without associated constant region(s). As noted, encoding nucleotide sequences may be obtained directly from antibody-producing cells of a mouse, or B cells may be immortalised or fused to generate hybridomas expressing the antibody, and encoding nucleic acid obtained from such cells. Optionally, nucleic acid encoding the variable domain(s) is then conjugated to a nucleotide sequence encoding a human heavy chain constant region and/or human light chain constant region, to provide nucleic acid encoding a human antibody heavy chain and/or human antibody light chain, e.g., encoding an antibody comprising both the heavy and light chain. As described elsewhere herein, this step is particularly useful where the immunised mammal produces chimaeric antibodies with non-human constant regions, which are preferably replaced with human constant regions to generate an antibody that will be less immunogenic when administered to humans as a medicament. Provision of particular human isotype constant regions is also significant for determining the effector function of the antibody, and a number of suitable heavy chain constant regions are discussed herein.

Other alterations to nucleic acid encoding the antibody heavy and/or light chain variable domain may be performed, such as mutation of residues and generation of variants, as described herein.

The isolated (optionally mutated) nucleic acid may be introduced into host cells, e.g., CHO cells as discussed. Host cells are then cultured under conditions for expression of the antibody, or of the antibody heavy and/or light chain variable domain, in any desired antibody format. Some possible antibody formats are described herein, e.g., whole immunoglobulins, antigen-binding fragments, and other designs.

Variable domain amino acid sequence variants of any of the VH and VL domains or CDRs whose sequences are specifically disclosed herein may be employed in accordance with the present invention, as discussed.

There are many reasons why it may be desirable to create variants, which include optimising the antibody sequence for large-scale manufacturing, facilitating purification, enhancing stability or improving suitability for inclusion in a desired pharmaceutical formulation. Protein engineering work can be performed at one or more target residues in the antibody sequence, e.g., to substituting one amino acid with an alternative amino acid (optionally, generating variants containing all naturally occurring amino acids at this position, with the possible exception of Cys and Met), and monitoring the impact on function and expression to determine the best substitution. It is in some instances undesirable to substitute a residue with Cys or Met, or to introduce these residues into a sequence, as to do so may generate difficulties in manufacturing—for instance through the formation of new intramolecular or intermolecular cysteine-cysteine bonds. Where a lead candidate has been selected and is being optimised for manufacturing and clinical development, it will generally be desirable to change its antigen-binding properties as little as possible, or at least to retain the affinity and potency of the parent molecule. However, variants may also be generated in order to modulate key antibody characteristics such as affinity, cross-reactivity or neutralising potency.

An antibody may comprise a set of H and/or L CDRs of any of the disclosed antibodies with one or more amino acid mutations within the disclosed set of H and/or L CDRs. The mutation may be an amino acid substitution, deletion or insertion. Thus for example there may be one or more amino acid substitutions within the disclosed set of H and/or L CDRs. For example, there may be up to 12, 11, 10, 9, 8, 7, 6, 5, 4, 3 or 2 mutations e.g. substitutions, within the set of H and/or L CDRs. For example, there may be up to 6, 5, 4, 3 or 2 mutations, e.g. substitutions, in HCDR3 and/or there may be up to 6, 5, 4, 3, or 2 mutations, e.g. substitutions, in LCDR3. An antibody may comprise the set of HCDRs, LCDRs or a set of 6 (H and L) CDRs shown for any STIM antibody herein or may comprise that set of CDRs with one or two conservative substitutions.

One or more amino acid mutations may optionally be made in framework regions of an antibody VH or VL domain disclosed herein. For example, one or more residues that differ from the corresponding human germline segment sequence may be reverted to germline. Human germline gene segment sequences corresponding to VH and VL domains of example anti-ICOS antibodies are indicated in Table E12-1, Table E12-2 and Table E12-3, and alignments of antibody VH and VL domains to corresponding germline sequences are shown in the drawings.

An antibody may comprise a VH domain that has at least 60, 70, 80, 85, 90, 95, 98 or 99% amino acid sequence identity with a VH domain of any of the antibodies shown in the appended sequence listing, and/or comprising a VL domain that has at least 60, 70, 80, 85, 90, 95, 98 or 99% amino acid sequence identity with a VL domain of any of those antibodies. Algorithms that can be used to calculate % identity of two amino acid sequences include e.g. BLAST, FASTA, or the Smith-Waterman algorithm, e.g. employing default parameters. Particular variants may include one or more amino acid sequence alterations (addition, deletion, substitution and/or insertion of an amino acid residue).

Alterations may be made in one or more framework regions and/or one or more CDRs. Variants are optionally provided by CDR mutagenesis. The alterations normally do not result in loss of function, so an antibody comprising a thus-altered amino acid sequence may retain an ability to bind ICOS. It may retain the same quantitative binding ability as an antibody in which the alteration is not made, e.g. as measured in an assay described herein. The antibody comprising a thus-altered amino acid sequence may have an improved ability to bind ICOS.

Alteration may comprise replacing one or more amino acid residue with a non-naturally occurring or non-standard amino acid, modifying one or more amino acid residue into a non-naturally occurring or non-standard form, or inserting one or more non-naturally occurring or non-standard amino acid into the sequence. Examples of numbers and locations of alterations in sequences of the invention are described elsewhere herein. Naturally occurring amino acids include the 20 “standard” L-amino acids identified as G, A, V, L, I, M, P, F, W, S, T, N, Q, Y, C, K, R, H, D, E by their standard single-letter codes. Non-standard amino acids include any other residue that may be incorporated into a polypeptide backbone or result from modification of an existing amino acid residue. Non-standard amino acids may be naturally occurring or non-naturally occurring.

The term “variant” as used herein refers to a peptide or nucleic acid that differs from a parent polypeptide or nucleic acid by one or more amino acid or nucleic acid deletions, substitutions or additions, yet retains one or more specific functions or biological activities of the parent molecule. Amino acid substitutions include alterations in which an amino acid is replaced with a different naturally-occurring amino acid residue. Such substitutions may be classified as “conservative”, in which case an amino acid residue contained in a polypeptide is replaced with another naturally occurring amino acid of similar character either in relation to polarity, side chain functionality or size. Such conservative substitutions are well known in the art. Substitutions encompassed by the present invention may also be “non-conservative”, in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments amino acid substitutions are conservative. Also encompassed within the term variant when used with reference to a polynucleotide or polypeptide, refers to a polynucleotide or polypeptide that can vary in primary, secondary, or tertiary structure, as compared to a reference polynucleotide or polypeptide, respectively (e.g., as compared to a wild-type polynucleotide or polypeptide).

In some aspects, one can use “synthetic variants”, “recombinant variants”, or “chemically modified” polynucleotide variants or polypeptide variants isolated or generated using methods well known in the art. “Modified variants” can include conservative or non-conservative amino acid changes, as described below. Polynucleotide changes can result in amino acid substitutions, additions, deletions, fusions and truncations in the polypeptide encoded by the reference sequence. Some aspects use include insertion variants, deletion variants or substituted variants with substitutions of amino acids, including insertions and substitutions of amino acids and other molecules) that do not normally occur in the peptide sequence that is the basis of the variant, for example but not limited to insertion of ornithine which do not normally occur in human proteins. The term “conservative substitution,” when describing a polypeptide, refers to a change in the amino acid composition of the polypeptide that does not substantially alter the polypeptide's activity. For example, a conservative substitution refers to substituting an amino acid residue for a different amino acid residue that has similar chemical properties (e.g., acidic, basic, positively or negatively charged, polar or nonpolar, etc.). Conservative amino acid substitutions include replacement of a leucine with an isoleucine or valine, an aspartate with a glutamate, or a threonine with a serine. Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984), incorporated by reference in its entirety.) In some embodiments, individual substitutions, deletions or additions that alter, add or delete a single amino acid or a small percentage of amino acids can also be considered “conservative substitutions” if the change does not reduce the activity of the peptide. Insertions or deletions are typically in the range of about 1 to 5 amino acids. The choice of conservative amino acids may be selected based on the location of the amino acid to be substituted in the peptide, for example if the amino acid is on the exterior of the peptide and expose to solvents, or on the interior and not exposed to solvents.

One can select the amino acid that will substitute an existing amino acid based on the location of the existing amino acid, including its exposure to solvents (i.e., if the amino acid is exposed to solvents or is present on the outer surface of the peptide or polypeptide as compared to internally localized amino acids not exposed to solvents). Selection of such conservative amino acid substitutions are well known in the art, for example as disclosed in Dordo et al, J. Mol Biol, 1999, 217, 721-739 and Taylor et al, J. Theor. Biol. 119(1986); 205-218 and S. French and B. Robson, J. Mol. Evol. 19(1983)171. Accordingly, one can select conservative amino acid substitutions suitable for amino acids on the exterior of a protein or peptide (i.e. amino acids exposed to a solvent), for example, but not limited to, the following substitutions can be used: substitution of Y with F, T with S or K, P with A, E with D or Q, N with D or G, R with K, G with N or A, T with S or K, D with N or E, I with L or V, F with Y, S with T or A, R with K, G with N or A, K with R, A with S, K or P.

In alternative embodiments, one can also select conservative amino acid substitutions encompassed suitable for amino acids on the interior of a protein or peptide, for example one can use suitable conservative substitutions for amino acids is on the interior of a protein or peptide (i.e. the amino acids are not exposed to a solvent), for example but not limited to, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, non-conservative amino acid substitutions are also encompassed within the term of variants.

The invention includes methods of producing antibodies containing VH and/or VL domain variants of the antibody VH and/or VL domains shown in the appended sequence listing. Such antibodies may be produced by a method comprising
(i) providing, by way of addition, deletion, substitution or insertion of one or more amino acids in the amino acid sequence of a parent antibody VH domain, an antibody VH domain that is an amino acid sequence variant of the parent antibody VH domain,
   wherein the parent antibody VH domain is the VH domain of any of antibodies STIM001, STIM002, STIM002-B, STIM003, STIM004, STIM005, STIM006, STIM007, STIM008 and STIM009 or a VH domain comprising the heavy chain complementarity determining regions of any of those antibodies,
(ii) optionally combining the VH domain thus provided with a VL domain, to provide a VH/VL combination, and
(iii) testing the VH domain or VH/VL domain combination thus provided to identify an antibody with one or more desired characteristics.

Desired characteristics include binding to human ICOS, binding to mouse ICOS, and binding to other non-human ICOS such as cynomolgus ICOS. Antibodies with comparable or higher affinity for human and/or mouse ICOS may be identified. Other desired characteristics include increasing effector T cell function indirectly, via depletion of immunosuppressive TRegs, or directly, via ICOS signalling activation on T effector cells. Identifying an antibody with a desired characteristic may comprise identifying an antibody with a functional attribute described herein, such as its affinity, cross-reactivity, specificity, ICOS receptor agonism, neutralising potency and/or promotion of T cell dependent killing, any of which may be determined in assays as described herein.

When VL domains are included in the method, the VL domain may be a VL domain of any of STIM001, STIM002, STIM002-B, STIM003, STIM004, STIM005, STIM006, STIM007, STIM008 or STIM009, or may be a variant provided by way of addition, deletion, substitution or insertion of one or more amino acids in the amino acid sequence of a parent VL domain, wherein the parent VL domain is the VL domain of any of STIM001, STIM002, STIM002-B, STIM003, STIM004, STIM005, STIM006, STIM007, STIM008 and STIM009 or a VL domain comprising the light chain complementarity determining regions of any of those antibodies.

Methods of generating variant antibodies may optionally comprise producing copies of the antibody or VH/VL domain combination. Methods may further comprise expressing the resultant antibody. It is possible to produce nucleotide sequences corresponding to a desired antibody VH and/or VL domain, optionally in one or more expression vectors. Suitable methods of expression, including recombinant expression in host cells, are set out in detail herein.

### Encoding Nucleic Acids and Methods of Expression

Isolated nucleic acid may be provided, encoding antibodies according to the present invention. Nucleic acid may be DNA and/or RNA. Genomic DNA, cDNA, mRNA or other RNA, of synthetic origin, or any combination thereof can encode an antibody.

The present invention provides constructs in the form of plasmids, vectors, transcription or expression cassettes which comprise at least one polynucleotide as above. Exemplary nucleotide sequences are included in the sequence listing. Reference to a nucleotide sequence as set out herein encompasses a DNA molecule with the specified sequence, and encompasses a RNA molecule with the specified sequence in which U is substituted for T, unless context requires otherwise.

The present invention also provides a recombinant host cell that comprises one or more nucleic acids encoding the antibody. Methods of producing the encoded antibody may comprise expression from the nucleic acid, e.g., by culturing recombinant host cells containing the nucleic acid. The antibody may thus be obtained, and may be isolated and/or purified using any suitable technique, then used as appropriate. A method of production may comprise formulating the product into a composition including at least one additional component, such as a pharmaceutically acceptable excipient.

Systems for cloning and expression of a polypeptide in a variety of different host cells are well known. Suitable host cells include bacteria, mammalian cells, plant cells, filamentous fungi, yeast and baculovirus systems and transgenic plants and animals.

The expression of antibodies and antibody fragments in prokaryotic cells is well established in the art. A common bacterial host is E. coli. Expression in eukaryotic cells in culture is also available to those skilled in the art as an option for production. Mammalian cell lines available in the art for expression of a heterologous polypeptide include Chinese hamster ovary (CHO) cells, HeLa cells, baby hamster kidney cells, NSO mouse melanoma cells, YB2/0 rat myeloma cells, human embryonic kidney cells, human embryonic retina cells and many others.

Vectors may contain appropriate regulatory sequences, including promoter sequences, terminator sequences, polyadenylation sequences, enhancer sequences, marker genes and other sequences as appropriate. Nucleic acid encoding an antibody can be introduced into a host cell. Nucleic acid can be introduced to eukaryotic cells by various methods, including calcium phosphate transfection, DEAE-Dextran, electroporation, liposome-mediated transfection and transduction using retrovirus or other virus, e.g. vaccinia or, for insect cells, baculovirus. Introducing nucleic acid in the host cell, in particular a eukaryotic cell may use a viral or a plasmid based system. The plasmid system may be maintained episomally or may be incorporated into the host cell or into an artificial chromosome. Incorporation may be either by random or targeted integration of one or more copies at single or multiple loci. For bacterial cells, suitable techniques include calcium chloride transformation, electroporation and transfection using bacteriophage. The introduction may be followed by expressing the nucleic acid, e.g., by culturing host cells under conditions for expression of the gene, then optionally isolating or purifying the antibody.

Nucleic acid of the invention may be integrated into the genome (e.g. chromosome) of the host cell. Integration may be promoted by inclusion of sequences that promote recombination with the genome, in accordance with standard techniques.

The present invention also provides a method that comprises using nucleic acid described herein in an expression system in order to express an antibody.

### Therapeutic Use

An antibody described herein may be used in a method of treatment of the human or animal body by therapy, in particular in the treatment of cancer in a patient, wherein the patient has a PD-L1 negative tumour or a tumour with low PD-L1 expression. The antibodies may find use in increasing effector T cell response, which is of benefit for a range of diseases or conditions, including treating cancers or solid tumours and in the context of vaccination. Increased Teff response may be achieved using an antibody that modulates the balance or ratio between Teffs and Tregs in favour of Teff activity.

Anti-ICOS antibodies may be used for depleting regulatory T cells and/or increasing effector T cell response in a patient, and may be administered to a patient to treat a disease or condition amenable to therapy by depleting regulatory T cells and/or increasing effector T cell response.

Generally speaking, the present invention relates to treatment of cancer that are PD-L1 negative or exhibit a low PD-L1 expression. In particular, the present invention relates to the treatment of cancer in patients having a tumour that is PD-L1 negative or exhibits a low PD-L1 expression. The methods comprise administration of a modulator of ICOS to the patient. The tumour cells and/or the tumour-associated immune cells may be PD-L1 negative or may exhibit low PD-L1 expression.

In some embodiments, the patient has or has had a tumour sample tested for PD-L1 expression. This testing may occur at screening, that is the patient is screened fro PD-L1 expression prior to treatment with the ICOS-modulator. In some embodiments, the invention relates to a method of selecting a patient for treatment for cancer, wherein the patient is selected for treatment with an anti-ICOS antibody and optionally an anti-PD-L1 antibody, irrespective of the PD-L1 expression status in a tumour sample from the patient, optionally wherein a sample of a tumour from the patient is determined to be PD-L1 negative or low expressing, or wherein PD-L1 expression status of a tumour sample from the patient is not determined prior to selecting the patient for treatment. Given the present invention provides methods of treating even no or low-expressing PD-L1 tumours, screening for PD-L1 expression might not be necessary. The method may optionally further comprising administering to the patient an ICOS modulator (for example an anti-ICOS antibody such as an agonistic anti-ICOS antibody) and optionally a PD-L1 inhibitor (such as an anti-PD-L1 or anti-PD-1 antibody).

In some embodiments, the cancer may be associated with infectious agents. The cancer may be a virally-induced cancer. In some embodiments, the virus associated with the virally-induced cancer may be selected from HBV, HCV, HPV (such as cervical cancer, oropharyngeal cancer), and EBV (such as Burkitts lymphomas, gastric cancer, Hodgkin's lymphoma, other EBV positive B cell lymphomas, nasopharyngeal carcinoma and post-transplant lymphoproliferative disease). In some embodiments, the cancer may be selected from the group consisting of head and neck squamous cell carcinoma, cervical cancer, anogenital cancer and oropharyngeal cancer.

In some embodiments, the patient has, or has had, a tumour sample tested for HPV. In some embodiments the tumour is HPV positive. In some embodiments, the tumour is HPV negative. This testing may occur at screening, that is the patient is screened for HPV prior to treatment with the ICOS-modulator, or the HPV status of the tumour may be determined from historical patient data. In some embodiments the methods further comprise a step of determining the HPV status of the tumour. A “HPV positive” tumor is deemed to be associated with or derived from HPV infection. A “HPV negative” tumor is deemed not to be associated with or derived from HPV infection. In some embodiments the tumour cells are PD-L1 negative or exhibit low PD-L1 expression and the tumour is HPV (Human papillomavirus) positive.

In some embodiments, the patient has undergone a test for an infection, for example HPV, HBV, HCV, or EBV infection. In some embodiments the patient has undergone a test for HPV infection. In some embodiments the patient has an HPV infection or has had an HPV infection. Determining if a patient has, or has had, an HPV infection may be using tests known in the art, for example, testing of cells taken from a sample from a patient or DNA analysis of a sample taken from the patient. In some embodiments, the methods further comprise a step of determining the HPV status of the patient.

In some embodiments, the present invention relates to treatment of cancer in a patient who has previously received treatment for the cancer, wherein the previous treatment for the cancer was administration of a PD-L1 inhibitor and the patient did not respond to the previous treatment or ceased responding to the previous treatment, comprising administering to the patient an modulator inhibitor of ICOS. In other words, the cancer may be or may be characterised as refractory to PD-L1 inhibitor treatment. In some embodiments, the cancer may be or may be characterised as refractor to PD-L1 immunotherapy (for example anti-PD-L1 antibody or anti-PD-1 antibody treatment). In some embodiments, the patient may have previously received PD-L1 inhibitor treatment as the sole immunotherapy. Generally the cancer will be or will have been characterised as a PD-L1 negative cancer or a cancer that exhibits low PD-L1 expression. The present invention therefore includes the use of ICOS modulators as second- or further-line treatment.

In some embodiments, the invention relates to treatment of patients having cancer who have previously been administered a kinase inhibitor (in addition or instead of a PD-L1 inhibitor). In some embodiments the patients may have received surgical treatment for the cancer (for example complete or partial tumour resection) and/or radiotherapy and/or chemotherapy. The chemotherapy may be docetaxel, fluorouracil, cisplatin, paclitaxel and/or nab-paclitaxel. The cancer may be or may have been characterised as refractory to one or all of the previous treatments, or may have stopped responding to the previous treatment(s). In some embodiments, the cancer is or has been characterised as refractory to PD-L1 inhibitor monotherapy treatment. In some embodiments, the cancer is or has been characterised as refractory to treatment with a PD-L1 inhibitor as the sole immunotherapy agent. In some embodiments, the cancer is or has been characterised as refractory to treatment with nivolumab.

In some embodiments, the methods comprise a step of determining the level of PD-L1 expression. This may be done on a tumour sample from the patient. In some embodiments, the methods comprise obtaining a tumour sample from the patient. In some embodiments, the methods may be conducted on a tumour sample previously obtained from the patient. Tumour samples may be any suitable samples for example a tumour tissue sample such as a tumour biopsy. The tumour sample may be a sample of tumour cells.

If a determination is made that the cancer is PD-L1 negative or exhibits low PD-L1 expression, the ICOS modulator (such as an agonistic anti-ICOS antibody) may be administered, or the patient may be recommended for such a treatment, or a report may be generated recommending the patient for such treatment (the present invention therefore extending to the provision of such reports).

A determination of PD-L1 expression status (i.e. whether the cancer or tumour is PD-L1 negative or exhibits low PD-L1 expression) may be determined by any suitable means. In some embodiments, the determination of PD-L1 expression status may be conducted on a tumour sample (for example a tumour biopsy). In some embodiments, the PD-L1 expression status may be determined by immunohistochemistry (IHC). The sample may be prepared for analysis using IHC, for example slicing and fixing.

The sample may be analysed to determined the number of cells in the tumour sample (for example the number of tumour cells and/or tumour-associated immune cells) that express PD-L1. In some embodiments, the method determines the ratio (e.g. percentage) of tumour cells in the tumour sample that express PD-L1 to tumours cells in the tumour sample that do not express PD-L1. In some embodiments, the method the method determines the ratio (e.g. percentage) of tumour cells in the tumour sample and tumour-associated immune cells in the tumour sample that express PD-L1 to total number of tumour and tumour-associated immune cells in the sample.

Generally speaking, the number of tumour and/or tumour-associated immune cells In the tumour sample that express PD-L1 will be considered representative of the tumour as a whole.

Generally, for a PD-L1 negative tumour (i.e. a tumour that does not express PD-L1), 0% of the cells (i.e. tumour cells and/or tumour-associated immune cells) will express PD-L1.

Different cut-off points may be employed to determine whether a cancer or tumour is a “low” PD-L1 expressing tumour. In some embodiments, the cancer or tumour may be considered a low PD-L1 expressing tumour when about 25% or less of the tumour cells (in the tumour tissue sample or sampled tumour cells) express PD-L1. In some embodiments, a cut-off of less than about 20%, less than about 15%, less than about 10%, less than about 5%, less that about 4%, less than about 3%, less than about 2% or less than about 1% of the tumour cells (in the tumour tissue sample or sampled tumour cells) express PD-L1. In some embodiments, the cancer or tumour may be considered a low PD-L1 expressing tumour when about 25% or less of the tumour-associated immune cells (in the tumour tissue sample or sampled tumour cells) express PD-L1. In some embodiments, a cut-off of less than about 20%, less than about 15%, less than about 10%, less than about 5%, less that about 4%, less than about 3%, less than about 2% or less than about 1% of the tumour-associated immune cells (in the tumour tissue sample or sampled tumour cells) express PD-L1. In some embodiments, the cancer or tumour may be considered a low PD-L1 expressing tumour when about 25% or less of the tumour cells and tumour-associated immune cells (in the tumour tissue sample or sampled tumour cells) express PD-L1. In some embodiments, a cut-off of less than about 20%, less than about 15%, less than about 10%, less than about 5%, less that about 4%, less than about 3%, less than about 2% or less than about 1% of the tumour cells and tumour-associated immune cells (in the tumour tissue sample or sampled tumour cells) express PD-L1. Generally, any non-tumour associated immune cells that may be present in the tumour sample or sample of tumour cells may be excluded (for example neutrophils).

The PD-L1 expression may be calculated or expressed as a percentage. In some embodiments, the percentage of PD-L1 expression (i.e. the percentage of analysed cells that express PD-L1) may be determined according to the following formula: (number of PD-L1 positive tumour cells in the tumour tissue sample or sample of tumour cells/total number of tumour cells in the tumour tissue sample or sample of tumour cells)×100. In some embodiments, the percentage of PD-L1 expression (i.e. the percentage of analysed cells that express PD-L1) may be determined according to the following formula: (number of PD-L1 positive tumour cells and number of PD-L1 positive tumour-associated immune cells in the tumour tissue sample or sample of tumour cells/total number of tumour cells and tumour-associated immune in the tumour tissue sample or sample of tumour cells)×100. Non-tumour associated immune cells (e.g. neutrophils) are generally excluded from the calculation.

The cancer or tumour may be a CD8+ cancer or tumour. In some embodiments, at least 50% of the T-cells in the tumour may be CD8+. CD8 expression status of a cancer or tumour (that is the CD8 expression status of the T cells in the tumour) may be determined by any suitable means, for example by IHC, such as on a tumour sample or sample or tumour cells. In some embodiments, in particular although not limited to embodiments in which expression status is determined using IHC performed on a slice of tumour, the tumour sample or sample of tumour cells may comprise at least 190 cells CD8+ T-cells per mm².

The cancer or tumour may be an ICOS+ cancer or tumour, i.e. a cancer or tumour comprising ICOS+ immune cells, e.g. T-cells (more specifically, ICOS+ Treg cells in the tumour microenvironment). In some embodiments, at least 50% of the T-cells (i.e. Tregs) in the tumour may be ICOS+. ICOS expression status of a cancer or tumour (that is the ICOS expression status of the T cells in the tumour) may be determined by any suitable means, for example by IHC, such as on a tumour sample or sample or tumour cells. In some embodiments, the patient may have increased levels of ICOS+ immune cells (such as ICOS+ regulatory T cells in the TME) following treatment with another therapeutic agent. In some embodiments, the methods comprise administering a therapeutic agent to the patient, determining that the patient has an increased level of ICOS-positive+ immune cells (such as ICOS+ regulatory T cells) following the treatment with said agent, and administering an modulator of ICOS (for example an anti-ICOS antibody such as an agonistic anti-ICOS antibody) to the patient to reduce the level of ICOS+ regulatory T cells. In some embodiments, wherein the therapeutic agent is IL-2 or an immunomodulatory antibody (e.g., anti-PDL-1, anti-PD-1 or anti-CTLA-4).

Tumour associated immune cells may also be referred to herein as tumour-infiltrating lymphocytes (TILs) or simply immune cells in the tumour or in the tumour microenvironment (TME).

An antibody disclosed herein, or a composition comprising such an antibody molecule or its encoding nucleic acid, may be used or provided for use in any such method. Use of the antibody, or of a composition comprising it or its encoding nucleic acid, for the manufacture of a medicament for use in any such method is also envisaged. The method typically comprises administering the antibody or composition to a mammal. Suitable formulations and methods of administration are described elsewhere herein.

The cancer may be a solid tumour, e.g., renal cell cancer (optionally renal cell carcinoma, e.g., clear cell renal cell carcinoma), head and neck cancer, melanoma (optionally malignant melanoma), non-small cell lung cancer (e.g., adenocarcinoma), bladder cancer, ovarian cancer, cervical cancer, gastric cancer, liver cancer, pancreatic cancer, breast cancer, testicular germ cell carcinoma, or the metastases of a solid tumour such as those listed, or it may be a liquid haematological tumour e.g., lymphoma (such as Hodgkin's lymphoma or Non-Hodgkin's lymphoma, e.g., diffuse large B-cell lymphoma, DLBCL) or leukaemia (e.g., acute myeloid leukaemia). An anti-ICOS antibody may enhance tumour clearance in melanoma, head and neck cancer and non-small cell lung cancer and other cancers with a moderate to high mutational load [26]. By enhancing patients' immune response to their neoplastic lesions, immunotherapy using an anti-ICOS antibody offers the prospect of durable cures or long-term remissions, potentially even in the context of late stage disease.

Cancers are a diverse group of diseases, but anti-ICOS antibodies offer the possibility of treating a range of different cancers by exploiting the patient's own immune system, which has the potential to kill any cancer cell through recognition of mutant or overexpressed epitopes that distinguish cancer cells from normal tissue. By modulating the Teff/Treg balance, anti-ICOS antibodies can enable and/or promote immune recognition and killing of cancer cells. While anti-ICOS antibodies are therefore useful therapeutic agents for a wide variety of cancers, there are particular categories of cancers for which anti-ICOS therapy is especially suited and/or where anti-ICOS therapy can be effective when other therapeutic agents are not.

One such group is cancer that is positive for expression of ICOS ligand. Cancer cells may acquire expression of ICOS ligand, as has been described for melanoma [27]. Expression of ICOS ligand may provide the cells with a selective advantage as the surface-expressed ligand binds ICOS on Tregs, promoting the expansion and activation of the Tregs and thereby suppressing the immune response against the cancer. Cancer cells expressing ICOS ligand may depend for their survival on this suppression of the immune system by Tregs, and would thus be vulnerable to treatment with anti-ICOS antibodies that target the Tregs. This applies also to cancers derived from cells that naturally express ICOS ligand. Continued expression of ICOS ligand by these cells again provides a survival advantage through immune suppression. A cancer expressing ICOS ligand may be derived from antigen-presenting cells such as B cells, dendritic cells and monocytes and may be a liquid haematological tumour such as those mentioned herein. Interestingly it has been shown that these types of cancer are also high in ICOS and FOXP3 expression (TCGA data)—see Example 25 of WO2018/029474. Example 20 of WO2018/029474 demonstrates efficacy of exemplary anti-ICOS antibodies in treating tumours derived from cancerous B cells (A20 syngeneic cells) that express ICOS ligand.

Accordingly, anti-ICOS antibodies can be used in methods of treating cancers that are positive for expression of ICOS ligand. Further, a cancer to be treated with anti-ICOS antibody according to the present invention may be one that is positive for expression of ICOS and/or FOXP3, and optionally also expresses ICOS ligand.

Patients may undergo testing to determine whether their cancer is positive for expression of the protein of interest (e.g., ICOS ligand, ICOS, FOXP3 and/or CD8) or is positive, negative, or low expression for PD-L1, for example by taking a test sample (e.g., tumour biopsy) from the patient and determining expression of the protein of interest. Patients whose cancer has been characterised as negative for PD-L1, or has been characterised as having a low PD-L1 expression, are selected for treatment. Optionally, patients whose cancer has been also been characterised as positive for expression of one, two or all such proteins of interest (.g., ICOS ligand, ICOS, FOXP3 and/or CD8) are selected for treatment with anti-ICOS antibody. As discussed elsewhere herein, anti-ICOS antibody may be used as a monotherapy or in combination with one or more other therapeutic agents.

Anti-ICOS antibodies also offer hope to patients whose cancers are refractory to treatment with antibodies or other drugs directed to immune checkpoint molecules such as CTLA-4, PD-1, PD-L1, CD137, GITR or CD73, but in particular to cancers that are refractory to PD-L1 inhibitors. These immunotherapies are effective against some cancers but in some cases a cancer may not respond, or it may become unresponsive to continued treatment with the antibody. In common with antibodies to immune checkpoint inhibitors, anti-ICOS antibodies modulate the patient's immune system—nevertheless an anti-ICOS antibody may succeed where such other antibodies fail. It is shown herein that animals carrying A20 B cell lymphomas could be treated with anti-ICOS antibodies to reduce growth of the tumour, shrink the tumour and indeed clear the tumour from the body, whereas treatment with an anti-PD-L1 antibody was no better than control. The A20 cell line has also been reported to be resistant to anti-CTLA-4 [28].

Accordingly, anti-ICOS antibodies can be used in methods of treating cancers that are refractory to treatment with one or more immunotherapies, such as (any or all of) an anti-CTLA-4 antibody, anti-PD1 antibody, anti-PD-L1 antibody, anti-CD137 antibody, anti-GITR antibody, or anti-CD73 antibody, although in particular refractory to PD-L1 inhibitors, such as anti-PD1 or anti-PD-L1 antibodies. A cancer may be characterised as being refractory to treatment with an antibody or other drug if treatment with that antibody or drug does not significantly reduce growth of the cancer, e.g., if a tumour continues to grow or does not reduce in size or if after a response period the tumour re-initiates its growth. Non-response to a therapeutic agent may be determined ex vivo by testing a sample (e.g., tumour biopsy sample) for cancer cell killing or growth inhibition, and/or in the clinical setting by observing (e.g., using an imaging technology, including MRI) that a patient treated with the therapy is not responding to treatment. Patients whose cancer has been characterised as refractory to treatment with such an immunotherapy are selected for treatment with anti-ICOS antibody.

Samples obtained from patients may thus be tested to determine surface expression of a protein of interest, for example ICOS ligand, ICOS, FOXP3 and/or a target receptor to which another therapeutic agent (e.g., anti-receptor antibody) is directed. Surface expression of ICOS ligand, ICOS, FOXP3 and/or lack or loss of surface expression of the target receptor is an indication that the cancer is susceptible to anti-ICOS antibody therapy. Anti-ICOS antibodies can be provided for administration to a patient whose cancer is characterised by surface expression of ICOS ligand, ICOS, FOXP3 and/or lack or loss of surface expression of a target receptor, optionally where the patient has been previously treated with anti-PD1, anti-PD-L1 or with an antibody to the target receptor and has not responded or has stopped responding to treatment with that antibody, as measured for example by continued or renewed cancer cell growth, e.g., increase in tumour size.

Any suitable method may be employed to determine whether cancer cells test positive for surface expression of a protein such as ICOS ligand, PD-L1 or other target receptors mentioned herein. A typical method is immunohistochemistry, where a sample of the cells (e.g., a tumour biopsy sample) is contacted with an antibody for the protein of interest, and binding of antibody is detected using a labelled reagent—typically a second antibody that recognises the Fc region of the first antibody and carries a detectable label such as a fluorescent marker. A sample may be declared to test positive for ICOS or PD-L1 where at least a certain percentage of cells are labelled, as visualised by cell staining or other detection of the label. The antibody will generally be used in excess. Reagent antibodies to the molecules of interest are available or may be generated by straightforward methods. To test for ICOS ligand, the antibody MAB1651 is currently available from R&D systems as a mouse IgG that recognises human ICOS ligand. To test for PD-L1, the antibody SP263, currently available from Roche as a rabbit monoclonal primary antibody that recognises human PD-L1, may be used. Detection of mRNA levels of the ICOS ligand or PD-L1 or target receptor of interest is an alternative technique [27].

A further indication that a tumour will respond to treatment with anti-ICOS antibody is the presence of Tregs in the tumour microenvironment. Activated Tregs are characterised by ICOS-high and Foxp3-high surface expression. The presence of Tregs in a tumour, especially in elevated numbers, provides a further basis on which a patient may be selected for treatment with anti-ICOS antibody. Tregs may be detected in a tumour biopsy sample ex vivo, for example by immunohistochemistry (assaying for co-expression of both Foxp3 and ICOS, using antibodies to the target protein followed by detection of labels, as described above) or by single cell dispersion of the sample for use in FACS with labelled antibodies to ICOS and Foxp3. FACS methods are exemplified in Example 17 and Example 18 of WO2018/029474. In some embodiments, treatment with the ICOS modulator (and optionally the PD-L1 inhibitor) may cause a reduction in the size of tumour (compared to the size of the tumour at the onset of treatment). In some embodiments, treatment with the ICOS modulator (and optionally the PD-L1 inhibitor) may inhibit tumour growth. In some embodiments, treatment with the ICOS modulator (and optionally the PD-L1 inhibitor) may result in stable disease. Stable disease may be regarded as the tumour does not grow in size by more than 20% since the onset of treatment and does not shrink in size by more than 30% since the onset of treatment. In some embodiments, treatment with the ICOS modulator (and optionally the PD-L1 inhibitor) may extend the survival of the patient and/or delay disease progression.

The ICOS modulators such as anti-ICOS antibodies may be used for treating cancers associated with infectious agents, such as virally-induced cancers e.g. cancers that are caused by infection with a virus. In this category are head and neck squamous cell carcinoma, cervical cancer, Merkel cell carcinoma and many others. Viruses associated with cancer include HBV, HCV, HPV (cervical cancer, oropharyngeal cancer), and EBV (Burkitts lymphomas, gastric cancer, Hodgkin's lymphoma, other EBV positive B cell lymphomas, nasopharyngeal carcinoma and post transplant lymphoproliferative disease). The International Agency for Research on Cancer (Monograph 100B) identified the following major cancer sites associated with infectious agents:
Stomach/Gastric: Helicobacter pylori
Liver: Hepatitis B virus, hepatitis C virus (HCV), Opisthorchis viverrini, Clonorchis sinensis
Cervix uteri: Human papillomavirus (HPV) with or without HIV
Anogenital (penile, vulva, vagina, anus): HPV with or without HIV
Nasopharynx: Epstein-Barr virus (EBV)
Oropharynx: HPV with or without tobacco or alcohol consumption
Kaposi's sarcoma: Human herpes virus type 8 with or without HIV
Non-Hodgkin lymphoma: H. pylori, EBV with or without HIV, HCV, human T-cell lymphotropic virus type 1
Hodgkin's lymphoma: EBV with or without HIV
Bladder: Schistosoma haematobium.

Antibodies according to the present invention may be used for treating cancer associated with or induced by any of these infectious agents, such as the cancers specified above.

In some embodiments, the cancer is liver cancer, renal cell cancer, head and neck cancer, melanoma, non small cell lung cancer, diffuse large B-cell lymphoma, breast cancer, penile cancer, pancreatic cancer or oesophageal cancer. In some embodiments, the liver cancer is hepatocellular carcinoma. In some embodiments the head and neck cancer is metastatic squamous cell carcinoma. In some embodiments, the breast cancer is triple negative breast cancer. The present invention may be particular relevant to solid cancers.

Stimulation of effector T cell response can also contribute to immunity against infectious disease and/or to recovery from infectious disease in a patient. Thus, an anti-ICOS antibody may be used for treating infectious disease by administering the antibody to a patient.

Infectious diseases include those caused by pathogens, e.g., bacterial, fungal, viral or protozoal pathogens, and treatment may be to promote immune response in a patient against the pathogen infection. An example of a bacterial pathogen is tuberculosis. Examples of viral pathogens are hepatitis B and HIV. Examples of protozoal pathogens are Plasmodium species, which cause malaria, such as P. falciparum.

The antibody may be used for treating infections, e.g., infection by any pathogen mentioned herein. Infection may be persistent or chronic infection. Infection may be localised or systemic. Extended contact between a pathogen and the immune system may lead to exhaustion of the immune system or development of tolerance (manifested for example through increased levels of Tregs, and tipping of the Treg:Teff balance in favour of Tregs) and/or to immune evasion by the pathogen, through evolution and modification of displayed pathogen antigens. These features reflect similar processes that are believed to occur in cancer. Anti-ICOS antibodies present a therapeutic approach to treating infection by a pathogen, e.g., chronic infection, through modulation of the Treg:Teff ratio in favour of Teff and/or other effects described herein.

Treatment may be of patients who have been diagnosed as having an infectious disease or an infection. Alternatively, treatment may be preventative, and administered to a patient to guard against contracting a disease, e.g., as a vaccine, as described elsewhere herein.

The present invention also provides an ICOS modulator for use in the treatment of cancer in a patient, wherein the patient has a PD-L1 negative tumour or a tumour with low PD-L1 expression. The present invention also provides an ICOS modulator for use in the treatment of cancer in a patient, wherein the patient has previously received treatment for the cancer and the patient did not respond to the previous treatment or ceased responding to the previous treatment, wherein the previous treatment for the cancer was a PD-L1 inhibitor. The present invention also provides use of an ICOS inhibitor modulator in the manufacture of a medicament for the treatment of cancer in a patient, wherein the patient has a PD-L1 negative tumour or a tumour with low PD-L1 expression. The present invention also provides use of an ICOS inhibitor in the manufacture of a medicament for the treatment of cancer in a patient, wherein the cancer is refractory to PD-L1 inhibitor treatment or has been characterised as being refractory to PD-L1 inhibitor treatment. The present invention also provides use of an ICOS inhibitor modulator in the manufacture of a medicament for the treatment of cancer in a patient, wherein the patient has previously received treatment for the cancer and the patient did not respond to the previous treatment or ceased responding to the previous treatment, wherein the previous treatment for the cancer was a PD-L1 inhibitor. In some embodiments, the ICOS modulator is for use in combination with a PD-L1 inhibitor. In some embodiments, the ICOS modulator is an agonistic anti-ICOS antibody. In some embodiments, the ICOS modulator is a bispecific antibody that is an anti-ICOS agonist and an anti-PD-L1 antagonist or a bispecific antibody that is an anti-ICOS agonist and an anti-PD-1 antagonist. Generally, the cancer (such as a solid cancer) will be a PD-L1 negative cancer or a cancer with low-PD-L1 expression.

### Combination Therapy

It may be advantageous to combine an anti-ICOS antibody with such an immunomodulator to enhance its therapeutic effects. In particular, the present invention relates in some embodiments to the combination of an ICOS modulator (such as an anti-ICOS antibody, for example an agonistic anti-ICOS antibody) and a PD-L1 inhibitor, i.e a PD-1 or PD-L1 binder that inhibits the binding of PD-L1 to PD-1 (for example an anti-PD-L1 or anti-PD-1 antibody). In combination therapies, the modulator of ICOS and inhibitor of PD-L1 may be administered simultaneously, separately or sequentially

A patient who has been treated with an immunomodulatory antibody (e.g., anti-PDL-1, anti-PD-1, anti-CTLA-4) may particularly benefit from treatment with an anti-ICOS antibody. One reason for this is that an immunomodulatory antibody may increase the number of ICOS-positive Tregs (e.g., intratumoural Tregs) in the patient. This effect is also observed with certain other therapeutic agents, such as recombinant IL-2. Anti-ICOS antibody may reduce and/or reverse a surge or rise in ICOS+ Tregs (e.g., intratumoural Tregs) resulting from treatment of the patient with another therapeutic agent. A patient selected for treatment with an anti-ICOS antibody may thus be one who has already received treatment with a first therapeutic agent, the first therapeutic agent being an antibody (e.g., immunomodulator antibody) or other agent (e.g., IL-2) that increases the number of ICOS+ Tregs in the patient.

Immunomodulators with which an anti-ICOS antibody may be combined include antibodies to any of: PDL1 (e.g., avelumab), PD-1 (e.g., pembrolizumab or nivolumab) or CTLA-4 (e.g., ipilimumab or tremelimumab). An anti-ICOS antibody may be combined with pidilizumab. In other embodiments, an anti-ICOS antibody is not administered in combination with anti-CTLA-4 antibody, and/or optionally is administered in combination with a therapeutic antibody that is not an anti-CTLA-4 antibody.

For example, an anti-ICOS antibody may be used in combination therapy with an anti-PDL1 antibody. Preferably, the anti-ICOS antibody is one that mediates ADCC, ADCP and/or CDC. Preferably, the anti-PDL1 antibody is one that mediates ADCC, ADCP and/or CDC. An example of such combination therapy is administration of an anti-ICOS antibody with an anti-PDL1 antibody wherein both antibodies have effector positive constant regions. Thus, the anti-ICOS antibody and the anti-PDL1 antibody may both be able to mediate ADCC, CDC and/or ADCP. Fc effector function and selection of constant regions is described in detail elsewhere herein, but as one example an anti-ICOS human IgG1 may be combined with an anti-PD-L1 human IgG1. The anti-ICOS antibody and/or the anti-PD-L1 antibody may comprise a wild type human IgG1 constant region. Alternatively, the effector positive constant region of an antibody may be one that is engineered for enhanced effector function, e.g., enhanced CDC, ADCC and/or ADCP. Example antibody constant regions, including wild type human IgG1 sequences and mutations that alter effector function, are discussed in detail elsewhere herein.

Anti-PDL1 antibodies with which an anti-ICOS antibody may be combined include:
Anti-PDL1 antibody that inhibits binding of PD-1 to PDL1 and/or inhibits PDL1, optionally as effector positive human IgG1;
Anti-PD-1 antibody that inhibits binding of PD-1 to PDL1 and/or PDL2;
Avelumab, a human IgG1 antibody which inhibits PD-1 binding to PDL-1. See WO2013/079174;
Durvalumab (or “MEDI4736”), a variant human IgG1 antibody having mutations L234A, L235A and 331. See WO2011/066389;
Atezolizumab, a variant human IgG1 antibody having mutations N297A, D356E and L358M. See US2010/0203056;
BMS-936559, a human IgG4 antibody comprising mutation S228P. See WO2007/005874.

Numerous further examples of anti-PD-L1 antibodies are disclosed herein and others are known in the art. Characterisation data for many of the anti-PD-L1 antibodies mentioned here has been published in U.S. Pat. Nos. 9,567,399 and 9,617,338, both incorporated by reference herein. Example anti-PD-L1 antibodies have VH and/or VL domains comprising the HCDRs and/or LCDRs of any of 1 D05, 84G09, 1 D05 HC mutant 1, 1 D05 HC mutant 2, 1D05 HC mutant 3, 1 D05 HC mutant 4, 1 D05 LC mutant 1, 1 D05 LC mutant 2, 1 D05 LC mutant 3, 411 B08, 411C04, 411D07, 385F01, 386H03, 389A03, 413D08, 413G05, 413F09, 414B06 or 416E01 as set out in U.S. Pat. No. 9,567,399 or U.S. Pat. No. 9,617,338. The antibody may comprise the VH and VL domain of any of these antibodies, and may optionally comprise a heavy and/or light chain having the heavy and/or light chain amino acid sequence of any of these antibodies. VH and VL domains of these anti-PD-L1 antibodies are further described elsewhere herein.

Further example anti-PD-L1 antibodies have VH and/or VL domains comprising the HCDRs and/or LCDRs of KN-035, CA-170, FAZ-053, M7824, ABBV-368, LY-3300054, GNS-1480, YW243.55.S70, REGN3504, or of an anti-PD-L1 antibody disclosed in any of WO2017/034916, WO2017/020291, WO2017/020858, WO2017/020801, WO2016/111645, WO2016/197367, WO2016/061142, WO2016/149201, WO2016/000619, WO2016/160792, WO2016/022630, WO2016/007235, WO2015/179654, WO2015/173267, WO2015/181342, WO2015/109124, WO2015/112805, WO2015/061668, WO2014/159562, WO2014/165082, WO2014/100079, WO2014/055897, WO2013/181634, WO2013/173223, WO2013/079174, WO2012/145493, WO2011/066389, WO2010/077634, WO2010/036959, WO2010/089411 and WO2007/005874. The antibody may comprise the VH and VL domain of any of these antibodies, and may optionally comprise a heavy and/or light chain having the heavy and/or light chain amino acid sequence of any of these antibodies. The anti-ICOS antibody which is used in combination therapy with anti-PD-L1 may be an antibody of the present invention as disclosed herein.

Alternatively, the anti-ICOS antibody may comprise the CDRs of, or a VH and/or VL domain of, an anti-ICOS antibody disclosed in any of the following publications:
WO2016154177, US2016304610—for example any of antibodies 7F12, 37A10, 35A9, 36E10, 16G10, 37A10S713, 37A10S714, 37A10S715, 37A10S716, 37A10S717, 37A10S718, 16G10S71, 16G10S72, 16G10S73, 16G10S83, 35A9S79, 35A9S710, or 35A9S89;
WO16120789, US2016215059—for example the antibody known as 422.2 and/or H2L5;
WO14033327, EP2892928, US2015239978—for example the antibody known as 314-8 and/or produced from hybridoma CNCM I-4180;
WO12131004, EP2691419, U.S. Pat. No. 9,376,493, US20160264666—for example the antibody Icos145-1 and/or antibody produced by hybridoma CNCM I-4179;
WO10056804—for example the antibody JMAb 136 or “136”;
WO9915553, EP1017723B1, U.S. Pat. Nos. 7,259,247, 7,132,099, 7,125,551, 7,306,800, 7,722,872,
WO05103086, EP1740617, U.S. Pat. Nos. 8,318,905, 8,916,155—for example the antibody MIC-944 or 9F3;
WO983821, U.S. Pat. No. 7,932,358B2, US2002156242, EP0984023, EP1502920, U.S. Pat. Nos. 7,030,225, 7,045,615, 7,279,560, 7,226,909, 7,196,175, 7,932,358, 8,389,690, WO02070010, EP1286668, EP1374901, U.S. Pat. Nos. 7,438,905, 7,438,905, WO0187981, EP1158004, U.S. Pat. Nos. 6,803,039, 7,166,283, 7,988,965, WO0115732, EP1125585, U.S. Pat. Nos. 7,465,445, 7,998,478—for example any JMAb antibody, e.g., any of JMAb-124, JMAb-126, JMAb-127, JMAb-128, JMAb-135, JMAb-136, JMAb-137, JMAb-138, JMAb-139, JMAb-140, JMAb-141, e.g., JMAb136; WO2014/089113—for example antibody 17G9;
WO12174338;
US2016145344;
WO11020024, EP2464661, US2016002336, US2016024211, U.S. Pat. No. 8,840,889;
U.S. Pat. No. 8,497,244.

The anti-ICOS antibody optionally comprises the CDRs of 37A10S713 as disclosed in WO2016154177. It may comprise the VH and VL domains of 37A10S713, and may optionally have the antibody heavy and light chains of 37A10S713.

Combination of an anti-ICOS antibody with an immunomodulator may provide an increased therapeutic effect compared with monotherapy, and may allow therapeutic benefit to be achieved with a lower dose of the immunomodulator(s). Thus, for example, an antibody (e.g., anti-PD-L1 antibody, optionally ipilimumab) that is used in combination with anti-ICOS antibody may be dosed at 3 mg/kg rather than a more usual dose of 10 mg/kg. The administration regimen of the anti-PD-L1 or other antibody may involve intravenous administration over a 90 minute period every 3 weeks for a total of 4 doses.

An anti-ICOS antibody may be used to increase the sensitivity of a tumour to treatment with an anti-PD-L1 antibody, which may be recognised as a reduction in the dose at which the anti-PD-L1 antibody exerts a therapeutic benefit. Thus, anti-ICOS antibody may be administered to a patient to reduce the dose of anti-PD-L1 antibody effective to treat cancer or a tumour in the patient. Administration of anti-ICOS antibody may reduce the recommended or required dosage of anti-PD-L1 antibody administration to that patient to, for example, 75%, 50%, 25%, 20%, 10% or less, compared with the dosage when anti-PD-L1 antibody is administered without anti-ICOS. The patient may be treated by administration of anti-ICOS antibody and anti-PD-L1 antibody in a combination therapy as described herein.

The benefit of combining anti-PD-L1 with anti-ICOS may extend to a reduction in dosage of each agent when compared with its use as a monotherapy. Anti-PD-L1 antibody may be used to reduce the dose at which anti-ICOS antibody exerts a therapeutic benefit, and thus may be administered to a patient to reduce the dose of anti-ICOS antibody effective to treat cancer or a tumour in the patient. Thus, an anti-PD-L1 antibody may reduce the recommended or required dosage of anti-ICOS antibody administration to that patient to, for example, 75%, 50%, 25%, 20%, 10% or less, compared with the dosage when anti-ICOS antibody is administered without anti-PD-L1. The patient may be treated by administration of anti-ICOS antibody and anti-PD-L1 antibody in a combination therapy as described herein.

As discussed in Example 22 of WO2018/029474, treatment with anti-PD-L1 antibody, especially antibody with effector positive Fc, appears not to increase the expression of ICOS on Teff cells. This is advantageous when administering such antibodies in combination with effector positive anti-ICOS antibodies, where an increase in ICOS expression on Teffs would undesirably render these cells more sensitive to depletion by the anti-ICOS antibody. In a combination with anti-PD-L1, anti-ICOS therapy may thus exploit a differential expression of ICOS on Teffs compared with Tregs, preferentially targeting the ICOS-high Tregs for depletion. This in turn relieves the suppression of TEffs and has a net effect of promoting the effector T cell response in a patient. The effect of targeting immune checkpoint molecules on expression of ICOS on T cells has also been studied previously—see FIG. S6C in ref. [29](supplementary materials), where treatment with CTLA-4 antibody and/or anti-PD-1 antibody was reported to increase the percentage of CD4+ Tregs expressing ICOS. The effect of a therapeutic agent on ICOS expression in Tregs and Teffs may be a factor in selection of appropriate agents for use in combination with anti-ICOS antibodies, noting that effect of the anti-ICOS antibody may be enhanced under conditions where there is high differential expression of ICOS on Tregs versus Teffs.

As described herein, a single dose of anti-ICOS antibody may be sufficient to provide therapeutic effect, especially in combination with other therapeutic agents such as anti-PD-L1 antibody. In tumour therapy, the underlying rationale for this single dose benefit may be that the anti-ICOS antibody mediates its effect, at least in part, by resetting or altering the microenvironment of the tumour sufficiently to render the tumour more sensitive to immune attack and/or to the effects of other immunomodulators such as those mentioned. Tumour microenvironment resetting is triggered through for example depletion of ICOS positive tumour infiltrating T-regs. So, for example, a patient may be treated with a single dose of an anti-ICOS antibody followed by one or multiple doses of anti-PD-L1 antibody. Over a period of treatment, for example six months or a year, the anti-ICOS antibody may be administered in a single dose while other agents, e.g., anti-PD-L1 antibody, are optionally administered multiple times over that treatment period, preferably with at least one such dose being administered subsequent to treatment with the anti-ICOS antibody.

Further examples of combination therapy include combination of anti-ICOS antibody with:
an antagonist of an adenosine A2A receptor (“A2AR inhibitor”);
a CD137 agonist (e.g., agonist antibody);
an antagonist of the enzyme indoleamine-2,3 dioxygenase, which catalyses the breakdown of tryptophan (“IDO inhibitor”). IDO is an immune checkpoint, activated in dendritic cells and macrophages, which contributes to immune suppression/tolerance.

Anti-ICOS antibodies may be used in combination therapy with IL-2 (e.g., recombinant IL-2 such as aldesleukin). The IL-2 may be administered at high dose (HD). Typical HD IL-2 therapy involves bolus infusion of over 500,000 IU/kg, e.g., bolus infusions of 600,000 or 720,000 IU/kg, per cycle of therapy, where 10-15 such bolus infusions are given at intervals of between 5-10 hours, e.g., up to 15 bolus infusions every 8 hours, and repeating the therapy cycle approximately every 14 to 21 days for up to 6 to 8 cycles. HD IL-2 therapy has been successful in treating tumours, especially melanoma (e.g., metastatic melanoma) and renal cell carcinoma, but its use is limited to the high toxicity of IL-2 which can cause severe adverse effects.

Treatment with high dose IL-2 has been shown to increase the population of ICOS-positive Tregs in cancer patients [30]. This increase in ICOS+ TRegs following the first cycle of HD IL-2 therapy was reported to correlate with worse clinical outcome—the higher the number of ICOS+ Tregs, the worse the prognosis. An IL-2 variant F42K has been proposed as an alternative therapy to avoid this undesirable increase in ICOS+ Treg cells [31]. However, another approach would be to exploit the increase in ICOS+T regs by using an antibody in accordance with the present invention as a second-line therapeutic agent.

It may be beneficial to combine IL-2 therapy with anti-ICOS antibodies, capitalising on the ability of anti-ICOS antibodies to target TRegs that highly express ICOS, inhibiting these cells and improving the prognosis for patients undergoing IL-2 therapy. Concomitant administration of IL-2 and anti-ICOS antibody may increase the response rate while avoiding or reducing adverse events in the treated patient population. The combination may permit IL-2 to be used at lower dose compared with IL-2 monotherapy, reducing the risk or level of adverse events arising from the IL-2 therapy, while retaining or enhancing clinical benefit (e.g., reduction of tumour growth, clearance of solid tumour and/or reduction of metastasis). In this way, addition of anti-ICOS can improve treatment of patients who are receiving IL-2, whether high-dose (HD) or low-dose (LD) IL-2.

Accordingly, one aspect of the invention provides a method of treating a patient by administering an anti-ICOS antibody to the patient, wherein the patient is also treated with IL-2, e.g., HD IL-2. Another aspect of the invention is an anti-ICOS antibody for use in treating a patient, wherein the patient is also treated with IL-2, e.g., HD IL-2. The anti-ICOS antibody may be used as a second-line therapy. Thus, the patient may be one who has been treated with IL-2, e.g., having received at least one cycle of HD IL-2 therapy, and who has an increased level of ICOS+ Tregs. Assays may be performed on samples of cancer cells, e.g., tumour biopsy samples, using immunohistochemistry or FACS as described elsewhere herein to detect cells positive for ICOS, Foxp3, ICOSL and optionally one or more further markers of interest. Methods may comprise determining that the patient has an increased level of ICOS+ Tregs (e.g., in peripheral blood, or in a tumour biopsy) following IL-2 treatment, where an increased level is indicative that the patient would benefit from treatment with the anti-ICOS antibody. The increase in Tregs may be relative to control (untreated) individuals or to the patient prior to IL-2 therapy. Such patients with elevated Tregs represent a group who may not benefit from continued IL-2 treatment alone, but for whom a combination of anti-ICOS antibody and IL-2 therapy, or treatment with anti-ICOS antibody alone, offers therapeutic benefit. Thus, following a positive determination that the patient has an increased level of ICOS+ Tregs, anti-ICOS antibody and/or further IL-2 therapy may be administered. Treatment with the anti-ICOS antibody may selectively target and deplete the ICOS+ Tregs relative to other T cell populations in such patients. This provides a therapeutic effect by relieving the immunosuppression mediated by these cells and thereby enhancing activity of Teffs against the target cells, e.g., tumour cells or infected cells.

Combination therapy with anti-ICOS antibodies and IL-2 may be used for any therapeutic indication described herein, and particularly for treating a tumour, e.g., melanoma such as metastatic melanoma, or renal cell carcinoma. Thus, in one example, the patient treated with an anti-ICOS antibody is one who presents with metastatic melanoma and has been treated with IL-2, e.g., HD IL-2 therapy or LD IL-2 therapy.

In general, where an anti-ICOS antibody is administered to a patient who has received treatment with a first therapeutic agent (e.g., immunomodulator antibody) or other agent (e.g., IL-2), the anti-ICOS antibody may be administered after a minimum period of, for example, 24 hours, 48 hours, 72 hours, 1 week or 2 weeks following administration of the first therapeutic agent. The anti-ICOS antibody may be administered within 2, 3, 4 or 5 weeks after administration of the first therapeutic agent. This does not exclude additional administrations of either agent at any time, although it may be desirable to minimise the number of treatments administered, for ease of compliance for patients and to reduce costs. Rather, the relative timing of the administrations will be selected to optimise their combined effect, the first therapeutic agent creating an immunological environment (e.g., elevated ICOS+ Tregs, or antigen release as discussed below) in which the effect of the anti-ICOS antibody is especially advantageous. Thus, sequential administration of the first therapeutic agent and then the anti-ICOS antibody may allow time for the first agent to act, creating in vivo conditions in which the anti-ICOS antibody can exhibit its enhanced effect. Various administration regimens, including simultaneous or sequential combination treatments, are described herein and can be utilised as appropriate. Where the first therapeutic agent is one that increases the number of ICOS+ Tregs in the patient, the treatment regimen for the patient may comprise determining that the patient has an increased number of ICOS+ Tregs, and then administering the anti-ICOS antibody.

As noted, use of anti-ICOS antibodies in combination therapy may provide advantages of reducing the effective dose of the therapeutic agents and/or countering adverse effects of therapeutic agents that increase ICOS+ Tregs in patients. Yet further therapeutic benefits may be achieved through selecting a first therapeutic agent that causes release of antigens from target cells through “immunological cell death”, and administering the first therapeutic agent in combination with an anti-ICOS antibody. As noted, administration of the anti-ICOS antibody may sequentially follow administration of the first therapeutic agent, administration of the two agents being separated by a certain time window as discussed above.

Immunological cell death is a recognised mode of cell death, contrasting with apoptosis. It is characterised by release of ATP and HMGB1 from the cell and exposure of calreticulin on the plasma membrane [32, 33].

Immunological cell death in a target tissue or in target cells promotes engulfment of the cell by an antigen-presenting cell, resulting in display of antigens from the target cell, which in turn induces antigen-specific Teff cells. Anti-ICOS antibody may increase the magnitude and/or duration of the Teff response by acting as an agonist of ICOS on the Teff cells. In addition, where the anti-ICOS antibody is Fc effector function enabled (e.g., a human IgG1 antibody), the anti-ICOS antibody may cause depletion of antigen-specific Tregs. Thus, through a combination of either or both of these effects, the balance between Teff and Treg cells is modulated in favour of enhancing Teff activity. Combination of an anti-ICOS antibody with a treatment that induces immunological cell death in a target tissue or cell type, such as in a tumour or in cancer cells, thereby promotes an immune response in the patient against the target tissue or cells, representing a form of vaccination in which the vaccine antigen is generated in vivo.

Accordingly, one aspect of the invention is a method of treating cancer in a patient by in vivo vaccination of the patient against their cancer cells. Another aspect of the invention is an anti-ICOS antibody for use in such a method. Anti-ICOS antibodies may be used in a method comprising:
treating the patient with a therapy that causes immunological cell death of the cancer cells, resulting in presentation of antigen to antigen-specific effector T cells, and
administering an anti-ICOS antibody to the patient, wherein the anti-ICOS antibody enhances the antigen-specific effector T cell response against the cancer cells.

Treatments that induce immunological cell death include radiation (e.g., ionising irradiation of cells using UVC light or γ rays), chemotherapeutic agents (e.g., oxaliplatin, anthracyclines such as doxorubicin, idarubicin or mitoxantrone, BK channel agonists such as phloretin or pimaric acid, bortezomib, cardiac glycosides, cyclophosphamide, GADD34/PP1 inhibitors with mitomycin, PDT with hypericin, polyinosinic-polycytidylic acid, 5-fluorouracil, gemcitabine, gefitnib, erlotinib, or thapsigargin with cisplatin) and antibodies to tumour-associated antigens. The tumour-associated antigen can be any antigen that is over-expressed by tumour cells relative to non-tumour cells of the same tissue, e.g., HER2, CD20, EGFR. Suitable antibodies include herceptin (anti-HER2), rituximab (anti-CD20), or cetuximab (anti-EGFR).

Thus, it is advantageous to combine an anti-ICOS antibody with one or more such treatments. Optionally, the anti-ICOS antibody is adminstered to a patient who has already received such treatment. The anti-ICOS antibody may be administered after a period of, for example, 24 hours, 48 hours, 72 hours, 1 week or 2 weeks following the treatment that induces immunological cell death, e.g., between 24 to 72 hours after the treatment. The anti-ICOS antibody may be administered within 2, 3, 4 or 5 weeks after the treatment. Other regimens for combination therapy are discussed elsewhere herein.

While “in vivo vaccination” has been described above, it is also possible to treat tumour cells to induce immunological cell death ex vivo, after which the cells may be reintroduced to the patient. Rather than administering the agent or treatment that induces immunological cell death directly to the patient, the treated tumour cells are administered to the patient. Treatment of the patient may be in accordance with administration regimens described above.

As already noted, a single dose of an anti-ICOS antibody may be sufficient to provide therapeutic benefit. Thus, in the methods of treatment described herein, the anti-ICOS antibody is optionally administered as a single dose. A single dose of anti-ICOS antibody may deplete Tregs in a patient, with consequent beneficial effects in diseases such as cancer. It has previously been reported that transient ablation of Tregs has anti-tumour effects, including reducing tumour progression, treating established tumours and metastases and extending survival, and that it can enhance the therapeutic effect of tumour irradiation [34]. Administration of a single dose of anti-ICOS may provide such Treg depletion, and may be used to enhance the effects of other therapeutic approaches used in combination, such as radiotherapy.

The present invention also provides a combination of an ICOS modulator and a PD-L1 inhibitor for use in the treatment of cancer in a patient, wherein the patient has a PD-L1 negative tumour or a tumour with low PD-L1 expression. The present invention also provides a combination of an ICOS modulator and a PD-L1 inhibitor for use in the treatment of cancer in a patient, wherein the patient has previously received treatment for the cancer and the patient did not respond to the previous treatment or ceased responding to the previous treatment, wherein the previous treatment for the cancer was a PD-L1 inhibitor. The present invention also provides a modulator of ICOS and an inhibitor of PD-L1 for use in the treatment of cancer in a patient, wherein the patient has a PD-L1 negative cancer or a cancer with low PD-L1 expression. The present invention also provides a modulator of ICOS and an inhibitor of PD-L1 for use in the treatment of cancer in a patient, wherein the patient has previously received treatment for the cancer and the patient did not respond to the previous treatment or ceased responding to the previous treatment, wherein the previous treatment for the cancer was a PD-L1 inhibitor. The present invention also provides use of a combination of an ICOS modulator and a PD-L1 inhibitor in the manufacture of a medicament for the treatment of cancer in a patient, wherein the patient has a PD-L1 negative tumour or a tumour with low PD-L1 expression. The present invention also provides use of a combination of an ICOS modulator and a PD-L1 inhibitor in the manufacture of a medicament for the treatment of cancer in a patient and the patient did not respond to the previous treatment or ceased responding to the previous treatment, wherein the patient has previously received treatment for the cancer, wherein the previous treatment for the cancer was a PD-L1 inhibitor. The present invention also provides use of a modulator of ICOS and an inhibitor of PD-L1 in the manufacture of a medicament for the treatment of cancer in a patient, wherein the patient has a PD-L1 negative cancer or a cancer with low PD-L1 expression. The present invention also provides use of a modulator of ICOS and an inhibitor of PD-L1 in the manufacture of a medicament for the treatment of cancer in a patient, wherein the patient has previously received treatment for the cancer, wherein the previous treatment for the cancer was a PD-L1 inhibitor. In some embodiments, the ICOS modulator is for use in combination with a PD-L1 inhibitor. In some embodiments, the ICOS modulator is an agonistic anti-ICOS antibody. In some embodiments, the ICOS modulator is a bispecific antibody that is an anti-ICOS agonist and an anti-PD-L1 antagonist or a bispecific antibody that is an anti-ICOS agonist and an anti-PD-1 antagonist. Generally, the cancer (such as a solid cancer) will be a PD-L1 negative cancer or a cancer with low-PD-L1 expression.

### Antibodies to PD-L1

An antibody to PD-L1 for use in combination with an anti-ICOS antibody, whether as a separate therapeutic agent or in a multispecific antibody as described herein, may comprise the antigen-binding site of any anti-PD-L1 antibody. Numerous examples of anti-PD-L1 antibodies are disclosed herein and others are known in the art. Characterisation data for many of the anti-PD-L1 antibodies mentioned here has been published in U.S. Pat. Nos. 9,567,399 and 9,617,338, both incorporated by reference herein.

1D05 has a heavy chain variable region (V_{H}) amino acid sequence of Seq ID No:33, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). The heavy chain nucleic acid sequence of the V_{H} domain is Seq ID No:34. 1D05 has a light chain variable region (V_{L}) amino acid sequence of Seq ID No:43, comprising the CDRL1 amino acid sequence of Seq ID No:37 (IMGT) or Seq ID No:40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The light chain nucleic acid sequence of the V_{L} domain is Seq ID No:44. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No: 526, Seq ID No:528, Seq ID No: 530, Seq ID No: 532 or Seq ID No: 534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:35 (heavy chain nucleic acid sequence Seq ID No:36). A full length light chain amino acid sequence is Seq ID No:45 (light chain nucleic acid sequence Seq ID No:46).

84G09 has a heavy chain variable (V_{H}) region amino acid sequence of Seq ID No:13, comprising the CDRH1 amino acid sequence of Seq ID No:7 (IMGT) or Seq ID No:10 (Kabat), the CDRH2 amino acid sequence of Seq ID No:8 (IMGT) or Seq ID No:11 (Kabat), and the CDRH3 amino acid sequence of Seq ID No:9 (IMGT) or Seq ID No:12 (Kabat). The heavy chain nucleic acid sequence of the V_{H} domain is Seq ID No:14. 84G09 has a light chain variable region (V_{L}) amino acid sequence of Seq ID No:23, comprising the CDRL1 amino acid sequence of Seq ID No:17 (IMGT) or Seq ID No:20 (Kabat), the CDRL2 amino acid sequence of Seq ID No:18 (IMGT) or Seq ID No:21 (Kabat), and the CDRL3 amino acid sequence of Seq ID No:19 (IMGT) or Seq ID No:22 (Kabat). The light chain nucleic acid sequence of the V_{L} domain is Seq ID No:24. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:15 (heavy chain nucleic acid sequence Seq ID No:16). A full length light chain amino acid sequence is Seq ID No:25 (light chain nucleic acid sequence Seq ID No:26).

1D05 HC mutant 1 has a heavy chain variable (V_{H}) region amino acid sequence of Seq ID No:47, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). 1D05 HC mutant 1 has a light chain variable region (V_{L}) amino acid sequence of Seq ID No:43, comprising the CDRL1 amino acid sequence of Seq ID No:37 (IMGT) or Seq ID No:40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The light chain nucleic acid sequence of the V_{L} domain is Seq ID No:44. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length light chain amino acid sequence is Seq ID No:45 (light chain nucleic acid sequence Seq ID No:46).

1D05 HC mutant 2 has a heavy chain variable (V_{H}) region amino acid sequence of Seq ID No:48, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). 1D05 HC mutant 2 has a light chain variable region (V_{L}) amino acid sequence of Seq ID No:43, comprising the CDRL1 amino acid sequence of Seq ID No:37 (IMGT) or Seq ID No:40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The light chain nucleic acid sequence of the V_{L} domain is Seq ID No:44. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length light chain amino acid sequence is Seq ID No:45 (light chain nucleic acid sequence Seq ID No:46).

1D05 HC mutant 3 has a heavy chain variable (V_{H}) region amino acid sequence of Seq ID No:49, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). 1D05 HC mutant 3 has a light chain variable region (V_{L}) amino acid sequence of Seq ID No:43, comprising the CDRL1 amino acid sequence of Seq ID No:37 (IMGT) or Seq ID No:40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The light chain nucleic acid sequence of the V_{L} domain is Seq ID No:44. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length light chain amino acid sequence is Seq ID No:45 (light chain nucleic acid sequence Seq ID No:46).

1D05 HC mutant 4 has a heavy chain variable (V_{H}) region amino acid sequence of Seq ID No:342, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). 1D05 HC mutant 4 has a light chain variable region (V_{L}) amino acid sequence of Seq ID No:43, comprising the CDRL1 amino acid sequence of Seq ID No:37 (IMGT) or Seq ID No:40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The light chain nucleic acid sequence of the V_{L} domain is Seq ID No:44. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length light chain amino acid sequence is Seq ID No:45 (light chain nucleic acid sequence Seq ID No:46).

1D05 LC mutant 1 has a heavy chain variable (V_{H}) region amino acid sequence of Seq ID No:33, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). The heavy chain nucleic acid sequence of the V_{H} domain is Seq ID No:34. 1D05 LC mutant 1 has a light chain variable region (V_{L}) amino acid sequence of Seq ID No:50, comprising the CDRL1 amino acid sequence of Seq ID No:37 (IMGT) or Seq ID No:40 (Kabat), and the CDRL3 amino acid sequence of Seq ID No:39 (IMGT) or Seq ID No:42 (Kabat). The CDRL2 sequence of 1D05 LC Mutant 1 is as defined by the Kabat or IMGT systems from the V_{L} sequence of Seq ID No:50. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205 or Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:35 (heavy chain nucleic acid sequence Seq ID No:36).

1D05 LC mutant 2 has a heavy chain variable (V_{H}) region amino acid sequence of Seq ID No:33, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). The heavy chain nucleic acid sequence of the V_{H} domain is Seq ID No:34. 1D05 LC mutant 2 has a light chain variable region (V_{L}) amino acid sequence of Seq ID No:51, comprising the CDRL1 amino acid sequence of Seq ID No:37 (IMGT) or Seq ID No:40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:35 (heavy chain nucleic acid sequence Seq ID No:36).

1D05 LC mutant 3 has a heavy chain variable (V_{H}) region amino acid sequence of Seq ID No:33, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). The heavy chain nucleic acid sequence of the V_{H} domain is Seq ID No:34. 1D05 LC mutant 3 has a light chain variable region (V_{L}) amino acid sequence of Seq ID No:298, comprising the CDRL1 amino acid sequence of Seq ID No:37 (IMGT) or Seq ID No:40 (Kabat), and the CDRL3 amino acid sequence of Seq ID No:39 (IMGT) or Seq ID No:42 (Kabat). The CDRL2 sequence of 1D05 LC Mutant 3 is as defined by the Kabat or IMGT systems from the V_{L} sequence of Seq ID No:298. The light chain nucleic acid sequence of the V_{L} domain is Seq ID No:44. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205 or Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:35 (heavy chain nucleic acid sequence Seq ID No:36). A full length light chain amino acid sequence is Seq ID No:45 (light chain nucleic acid sequence Seq ID No:46).

411B08 has a heavy chain variable (V_{H}) region amino acid sequence of Seq ID No:58, comprising the CDRH1 amino acid sequence of Seq ID No:52 (IMGT) or Seq ID No:55 (Kabat), the CDRH2 amino acid sequence of Seq ID No:53 (IMGT) or Seq ID No:56 (Kabat), and the CDRH3 amino acid sequence of Seq ID No:54 (IMGT) or Seq ID No:57 (Kabat). The heavy chain nucleic acid sequence of the V_{H} domain is Seq ID No:59. 411 B08 has a light chain variable region (V_{L}) amino acid sequence of Seq ID No:68, comprising the CDRL1 amino acid sequence of Seq ID No:62 (IMGT) or Seq ID No:65 (Kabat), the CDRL2 amino acid sequence of Seq ID No:63 (IMGT) or Seq ID No:66 (Kabat), and the CDRL3 amino acid sequence of Seq ID No:64 (IMGT) or Seq ID No:67 (Kabat). The light chain nucleic acid sequence of the V_{L} domain is Seq ID No:69. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:60 (heavy chain nucleic acid sequence Seq ID No:61). A full length light chain amino acid sequence is Seq ID No:70 (light chain nucleic acid sequence Seq ID No:71).

411C04 has a heavy chain variable (V_{H}) region amino acid sequence of Seq ID No:78, comprising the CDRH1 amino acid sequence of Seq ID No:72 (IMGT) or Seq ID No:75 (Kabat), the CDRH2 amino acid sequence of Seq ID No:73 (IMGT) or Seq ID No:76 (Kabat), and the CDRH3 amino acid sequence of Seq ID No:74 (IMGT) or Seq ID No:77 (Kabat). The heavy chain nucleic acid sequence of the V_{H} domain is Seq ID No:79. 411C04 has a light chain variable region (V_{L}) amino acid sequence of Seq ID No:88, comprising the CDRL1 amino acid sequence of Seq ID No:82 (IMGT) or Seq ID No:85 (Kabat), the CDRL2 amino acid sequence of Seq ID No:83 (IMGT) or Seq ID No:86 (Kabat), and the CDRL3 amino acid sequence of Seq ID No:84 (IMGT) or Seq ID No:87 (Kabat). The light chain nucleic acid sequence of the V_{L} domain is Seq ID No:89. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:80 (heavy chain nucleic acid sequence Seq ID No:81). A full length light chain amino acid sequence is Seq ID No:90 (light chain nucleic acid sequence Seq ID No:91).

411D07 has a heavy chain variable (V_{H}) region amino acid sequence of Seq ID No:98, comprising the CDRH1 amino acid sequence of Seq ID No:92 (IMGT) or Seq ID No:95 (Kabat), the CDRH2 amino acid sequence of Seq ID No:93 (IMGT) or Seq ID No:96 (Kabat), and the CDRH3 amino acid sequence of Seq ID No:94 (IMGT) or Seq ID No:97 (Kabat). The heavy chain nucleic acid sequence of the V_{H} domain is Seq ID No:99. 411D07 has a light chain variable region (V_{L}) amino acid sequence of Seq ID No:108, comprising the CDRL1 amino acid sequence of Seq ID No:102 (IMGT) or Seq ID No:105 (Kabat), the CDRL2 amino acid sequence of Seq ID No:103 (IMGT) or Seq ID No:106 (Kabat), and the CDRL3 amino acid sequence of Seq ID No:104 (IMGT) or Seq ID No:107 (Kabat). The light chain nucleic acid sequence of the V_{L} domain is Seq ID No:109. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:100 (heavy chain nucleic acid sequence Seq ID No:101). A full length light chain amino acid sequence is Seq ID No: 110 (light chain nucleic acid sequence Seq ID No:111).

385F01 has a heavy chain variable (V_{H}) region amino acid sequence of Seq ID No: 118, comprising the CDRH1 amino acid sequence of Seq ID No:112 (IMGT) or Seq ID No:115 (Kabat), the CDRH2 amino acid sequence of Seq ID No: 113 (IMGT) or Seq ID No: 116 (Kabat), and the CDRH3 amino acid sequence of Seq ID No: 114 (IMGT) or Seq ID No: 117 (Kabat). The heavy chain nucleic acid sequence of the V_{H} domain is Seq ID No:119. 385F01 has a light chain variable region (V_{L}) amino acid sequence of Seq ID No:128, comprising the CDRL1 amino acid sequence of Seq ID No:122 (IMGT) or Seq ID No:125 (Kabat), the CDRL2 amino acid sequence of Seq ID No:123 (IMGT) or Seq ID No:126 (Kabat), and the CDRL3 amino acid sequence of Seq ID No:124 (IMGT) or Seq ID No:127 (Kabat). The light chain nucleic acid sequence of the V_{L} domain is Seq ID No:129. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:120 (heavy chain nucleic acid sequence Seq ID No:121). A full length light chain amino acid sequence is Seq ID No:130 (light chain nucleic acid sequence Seq ID No:131).

386H03 has a heavy chain variable (V_{H}) region amino acid sequence of Seq ID No:158, comprising the CDRH1 amino acid sequence of Seq ID No:152 (IMGT) or Seq ID No:155 (Kabat), the CDRH2 amino acid sequence of Seq ID No:153 (IMGT) or Seq ID No:156 (Kabat), and the CDRH3 amino acid sequence of Seq ID No:154 (IMGT) or Seq ID No:157 (Kabat). The heavy chain nucleic acid sequence of the V_{H} domain is Seq ID No:159. 386H03 has a light chain variable region (V_{L}) amino acid sequence of Seq ID No:168, comprising the CDRL1 amino acid sequence of Seq ID No:162 (IMGT) or Seq ID No:165 (Kabat), the CDRL2 amino acid sequence of Seq ID No:163 (IMGT) or Seq ID No:166 (Kabat), and the CDRL3 amino acid sequence of Seq ID No:164 (IMGT) or Seq ID No:167 (Kabat). The light chain nucleic acid sequence of the V_{L} domain is Seq ID No:169. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:160 (heavy chain nucleic acid sequence Seq ID No:161). A full length light chain amino acid sequence is Seq ID No:170 (light chain nucleic acid sequence Seq ID No:171).

389A03 has a heavy chain variable (V_{H}) region amino acid sequence of Seq ID No:178, comprising the CDRH1 amino acid sequence of Seq ID No:172 (IMGT) or Seq ID No:175 (Kabat), the CDRH2 amino acid sequence of Seq ID No:173 (IMGT) or Seq ID No:176 (Kabat), and the CDRH3 amino acid sequence of Seq ID No:174 (IMGT) or Seq ID No:177 (Kabat). The heavy chain nucleic acid sequence of the V_{H} domain is Seq ID No:179. 389A03 has a light chain variable region (V_{L}) amino acid sequence of Seq ID No:188, comprising the CDRL1 amino acid sequence of Seq ID No:182 (IMGT) or Seq ID No:185 (Kabat), the CDRL2 amino acid sequence of Seq ID No:183 (IMGT) or Seq ID No:186 (Kabat), and the CDRL3 amino acid sequence of Seq ID No:184 (IMGT) or Seq ID No:187 (Kabat). The light chain nucleic acid sequence of the V_{L} domain is Seq ID No:189. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:180 (heavy chain nucleic acid sequence Seq ID No:181). A full length light chain amino acid sequence is Seq ID No:190 (light chain nucleic acid sequence Seq ID No:191).

413D08 has a heavy chain variable (V_{H}) region amino acid sequence of Seq ID No:138, comprising the CDRH1 amino acid sequence of Seq ID No:132 (IMGT) or Seq ID No:135 (Kabat), the CDRH2 amino acid sequence of Seq ID No:133 (IMGT) or Seq ID No:136 (Kabat), and the CDRH3 amino acid sequence of Seq ID No:134 (IMGT) or Seq ID No:137 (Kabat). The heavy chain nucleic acid sequence of the V_{H} domain is Seq ID No:139. 413D08 has a light chain variable region (V_{L}) amino acid sequence of Seq ID No:148, comprising the CDRL1 amino acid sequence of Seq ID No:142 (IMGT) or Seq ID No:145 (Kabat), the CDRL2 amino acid sequence of Seq ID No:143 (IMGT) or Seq ID No:146 (Kabat), and the CDRL3 amino acid sequence of Seq ID No:144 (IMGT) or Seq ID No:147 (Kabat). The light chain nucleic acid sequence of the V_{L} domain is Seq ID No:149. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No: 140 (heavy chain nucleic acid sequence Seq ID No:141). A full length light chain amino acid sequence is Seq ID No:150 (light chain nucleic acid sequence Seq ID No:151).

413G05 has a heavy chain variable (V_{H}) region amino acid sequence of Seq ID No:244, comprising the CDRH1 amino acid sequence of Seq ID No:238 (IMGT) or Seq ID No:241 (Kabat), the CDRH2 amino acid sequence of Seq ID No:239 (IMGT) or Seq ID No:242 (Kabat), and the CDRH3 amino acid sequence of Seq ID No:240 (IMGT) or Seq ID No:243 (Kabat). The heavy chain nucleic acid sequence of the V_{H} domain is Seq ID No:245. 413G05 has a light chain variable region (V_{L}) amino acid sequence of Seq ID No:254, comprising the CDRL1 amino acid sequence of Seq ID No:248 (IMGT) or Seq ID No:251 (Kabat), the CDRL2 amino acid sequence of Seq ID No:249 (IMGT) or Seq ID No:252 (Kabat), and the CDRL3 amino acid sequence of Seq ID No:250 (IMGT) or Seq ID No:253 (Kabat). The light chain nucleic acid sequence of the V_{L} domain is Seq ID No:255. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:246 (heavy chain nucleic acid sequence Seq ID No:247). A full length light chain amino acid sequence is Seq ID No:256 (light chain nucleic acid sequence Seq ID No:257).

413F09 has a heavy chain variable (V_{H}) region amino acid sequence of Seq ID No:264, comprising the CDRH1 amino acid sequence of Seq ID No:258 (IMGT) or Seq ID No:261 (Kabat), the CDRH2 amino acid sequence of Seq ID No:259 (IMGT) or Seq ID No:262 (Kabat), and the CDRH3 amino acid sequence of Seq ID No:260 (IMGT) or Seq ID No:263 (Kabat). The heavy chain nucleic acid sequence of the V_{H} domain is Seq ID No:265. 413F09 has a light chain variable region (V_{L}) amino acid sequence of Seq ID No:274, comprising the CDRL1 amino acid sequence of Seq ID No:268 (IMGT) or Seq ID No:271 (Kabat), the CDRL2 amino acid sequence of Seq ID No:269 (IMGT) or Seq ID No:272 (Kabat), and the CDRL3 amino acid sequence of Seq ID No:270 (IMGT) or Seq ID No:273 (Kabat). The light chain nucleic acid sequence of the V_{L} domain is Seq ID No:275. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:266 (heavy chain nucleic acid sequence Seq ID No:267). A full length light chain amino acid sequence is Seq ID No:276 (light chain nucleic acid sequence Seq ID No:277).

414B06 has a heavy chain variable (V_{H}) region amino acid sequence of Seq ID No:284, comprising the CDRH1 amino acid sequence of Seq ID No:278 (IMGT) or Seq ID No:281 (Kabat), the CDRH2 amino acid sequence of Seq ID No:279 (IMGT) or Seq ID No:282 (Kabat), and the CDRH3 amino acid sequence of Seq ID No:280 (IMGT) or Seq ID No:283 (Kabat). The heavy chain nucleic acid sequence of the V_{H} domain is Seq ID No:285. 414B06 has a light chain variable region (V_{L}) amino acid sequence of Seq ID No:294, comprising the CDRL1 amino acid sequence of Seq ID No:288 (IMGT) or Seq ID No:291(Kabat), the CDRL2 amino acid sequence of Seq ID No:289 (IMGT) or Seq ID No:292 (Kabat), and the CDRL3 amino acid sequence of Seq ID No:290 (IMGT) or Seq ID No:293 (Kabat). The light chain nucleic acid sequence of the V_{L} domain is Seq ID No:295. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:286 (heavy chain nucleic acid sequence Seq ID No:287). A full length light chain amino acid sequence is Seq ID No:296 (light chain nucleic acid sequence Seq ID No:297).

416E01 has a heavy chain variable region (V_{H}) amino acid sequence of Seq ID No:349, comprising the CDRH1 amino acid sequence of Seq ID No:343 (IMGT) or Seq ID No:346 (Kabat), the CDRH2 amino acid sequence of Seq ID No:344 (IMGT) or Seq ID No:347 (Kabat), and the CDRH3 amino acid sequence of Seq ID No:345 (IMGT) or Seq ID No:348 (Kabat). The heavy chain nucleic acid sequence of the V_{H} domain is Seq ID No:350. 416E01 has a light chain variable region (V_{L}) amino acid sequence of Seq ID No:359, comprising the CDRL1 amino acid sequence of Seq ID No:353 (IMGT) or Seq ID No:356 (Kabat), the CDRL2 amino acid sequence of Seq ID No:354 (IMGT) or Seq ID No:357 (Kabat), and the CDRL3 amino acid sequence of Seq ID No:355 (IMGT) or Seq ID No:358 (Kabat). The light chain nucleic acid sequence of the V_{L} domain is Seq ID No:360. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:351 (heavy chain nucleic acid sequence Seq ID No:352). A full length light chain amino acid sequence is Seq ID No:361 (light chain nucleic acid sequence Seq ID No:362).

In some embodiments, the anti-PD-L1 antibody is an anti-PD-L1 antibody selected from the group consisting of atezoliumab (Roche), avelumab (Merck), durvalumab/Medi4736 (Medimmune), KN035, CK-301, AUNP12, CA-170, BMS-936559/MDX-1105 (BMS), FAZ-053 M7824, ABBV-368, LY-3300054, GNS-1480, YW243.55.S70, REGN3504 and any of the PD-L1 antibodies disclosed in WO2017/220990, WO2017/034916, WO2017/020291, WO2017/020858, WO2017/020801, WO2016/111645, WO2016/197367, WO2016/061142, WO2016/149201, WO2016/000619, WO2016/160792, WO2016/022630, WO2016/007235, WO2015/179654, WO2015/173267, WO2015/181342, WO2015/109124, WO2015/112805, WO2015/061668, WO2014/159562, WO2014/165082, WO2014/100079, WO2014/055897, WO2013/181634, WO2013/173223, WO2013/079174, WO2012/145493, WO2011/066389, WO2010/077634, WO2010/036959, WO2010/089411 or WO2007/005874.

### Antibodies to PD-1

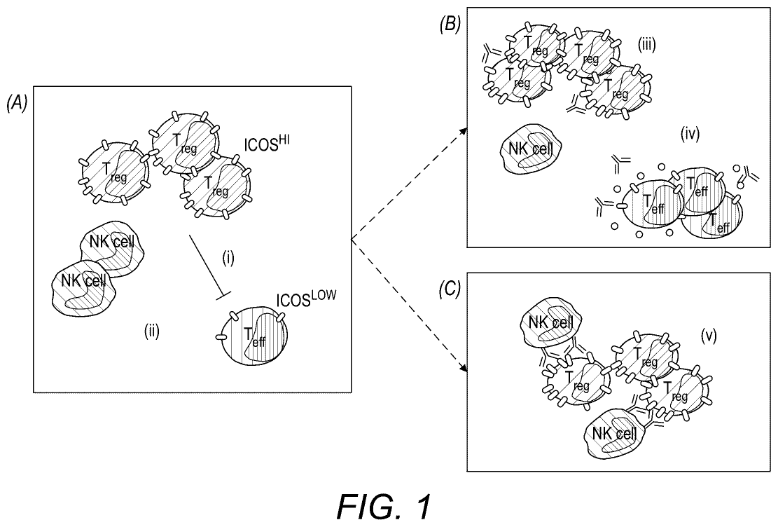

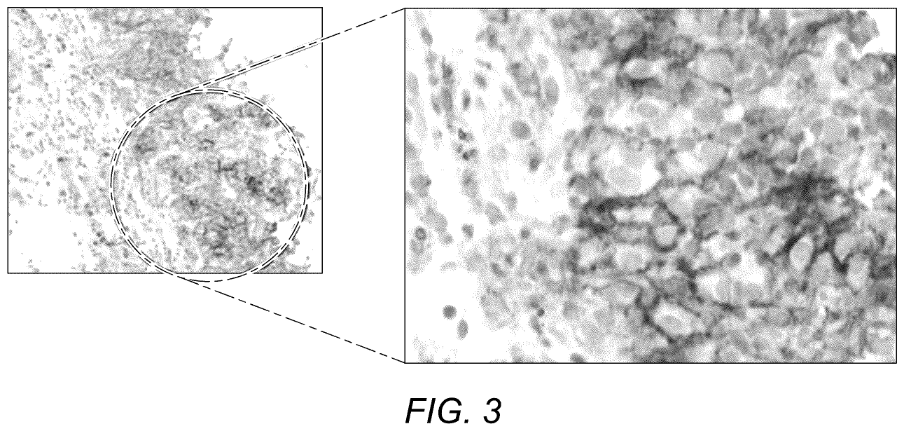

In some embodiments, the invention uses anti-PD-1 antibodies, for example an anti-PD-1 antibody selected from the group consisting of pembrolizumab, nivolumab, cemiplimab, JTX-401, spartalizumab (PDR001), camrelizumab (SHR1210), sintilimab (1B1308), tislelizumab (BGB-A317), toripalimab (JS 001), dostarlimab (TSR-042, WBP-285), INCMGA00012 (MGA012), AMP-224 and AMP-514, MEDI-0680/AMP514, PDR001, Lambrolizumab, BMS-936558, REGN2810, BGB-A317, BGB-108, PDR-001, SHR-1210, JS-001, JNJ-63723283, AGEN-2034, PF-06801591, genolimzumab, MGA-012 (INCMGA00012), IBI-308, BCD-100, TSR-042 ANA011, AUNP-12, KD033, MCLA-134, mDX400, muDX400, STI-A1110, AB011, 244C8, 388D4, XCE853, or pidilizumab/CT-011, or from any one of the anti-PD-1 antibodies described in WO2015/112800 & US2015/0203579 (including the antibodies in Tables 1 to 3), U.S. Pat. Nos. 9,394,365, 5,897,862 and 7,488,802, WO2017/087599 (including antibody SSI-361 and SHB-617), WO2017/079112, WO2017/071625 (including deposit C2015132, hybridoma LT004, and antibodies 6F5/6 F5 (Re), 6F5H1 L1 and 6F5 H2L2), WO2017/058859 (including PD1AB-1 to PD1AB-6), WO2017/058115 (including 67D9, c67D9, and hu67D9), WO2017/055547 (including 12819.15384, 12748.15381, 12748.16124, 12865.15377, 12892.15378, 12796.15376, 12777.15382, 12760.15375 and 13112.15380), WO2017/040790 (including AGEN2033w, AGEN2034w, AGEN2046w, AGEN2047w, AGEN2001w and AGEN2002w), WO2017/025051 & WO2017/024515 (including 1.7.3 hAb, 1.49.9 hAb, 1.103.11 hAb, 1.103.11-v2 hAb, 1.139.15 hAb and 1.153.7 hAb), WO2017/025016 & WO2017/024465 (including antibody A to antibody I), WO2017/020858 & WO2017/020291 (including 1.4.1, 1.14.4, 1.20.15 and 1.46.11), WO2017/019896 & WO2015/112900 & US2015/0210769 (including BAP049-hum01 to BAP049-hum16 and BAP049-Clone-A to BAP049-Clone-E), WO2017/019846 (including PD-1 mAb 1 to PD-1 mAb 15), WO2017/016497 (including MHC723, MHC724, MHC725, MHC728, MHC729, m136-M13, m136-M19, m245-M3, m245-M5 and m136-M14), WO2016/201051 (including antibody EH12.2H7, antibody hPD-1 mAb2, antibody hPD-1 mAb7, antibody hPD-1 mAb9, antibody hPD-1 mAb15, or an anti-PD-1 antibody selected from Table 1), WO2016/197497 (including DFPD1-1 to DFPD1-13), WO2016/197367 (including 2.74.15 and 2.74.15.hAb4 to 2.74.15.hAb8), WO2016/196173 (including the antibodies in Table 5, and FIGS. 1-5), WO2016/127179 (including R3A1, R3A2, R4B3, and R3D6), WO2016/077397 (including the antibodies described in Table 1 of Example 9), WO2016/106159 (including the murine antibodies in Table 3 of Example 2 and the humanised antibodies in Tables 7, 8 and 9 of Example 3), WO2016/092419 (including C1, C2, C3, EH12.1, mAb7-G4, mAb15-G4, mAb-AAA, mAb15-AAA), WO2016/068801 (including clone A3 and its variants and the other antibodies described in FIGS. 1 to 4), WO2016/014688 (including 10D1, 4C10, 7D3, 13F1, 15H5, 14A6, 22A5, 6E1, 5A8, 7A4, and 7A4D and the humanised antibodies of Examples 9/10), WO2016/015685 (including 10F8, BA08-1, BA-08-2 and 15H6), WO2015/091911 & WO2015/091910 (including the anti-canine PD-1 antibodies in Examples 2, 3 and 4), WO2015/091914 (including the anti-canine PD-1 antibodies in Table 3), WO2015/085847 (including mAb005, H005-1 to H005-4), WO2015/058573 (including cAB7), WO2015/036394 (including LOPD180), WO2015/035606 (including the antibodies in Table 1 of Example 2, in Tables 14, 15 and 16 of Example 7 and in tables 20, 21 and 22 of Example 11), WO2014/194302 (including GA2, RG1B3, RG1H10, RG2A7, RG2H10, SH-A4, RG4A6, GA1, GB1, GB6, GH1, A2, C7, H7, SH-A4, SH-A9, RG1H11, and RG6B), WO2014/179664 (including 9A2, 10B11, 6E9, APE1922, APE1923, APE1924, APE1950, APE1963 and APE2058), WO2014/206107 (including clone 1, 10, 11, 55, 64, 38, 39, 41 and 48), WO2012/135408 (including h409A11, h409A16, and h409A17), WO2012/145493 (including antibodies 1E3, 1E8, 1H3 and h1H3 Var 1 to h1H3 Var 14), WO2011/110621 (including antibody 949 and the modified versions disclosed in FIGS. 1 to 11), WO2011/110604 (including antibody 948 and the modified versions disclosed in FIGS. 3 to 11), WO2010/089411 (including CNCM deposit number 1-4122, 1-4080 or 1-4081), WO2010/036959 (including the antibodies in Table 1 of Example 1), WO2010/029435 & WO2010/029434 (including clones 2, 10 and 19), WO2008/156712 (including hPD-1.08A, hPD-1.09A, h409A11, h409A16 and h409A17 and the antibodies described in Example 2, Table H, Example 4 and table IV), WO2006/121168 (including clones 17D8, 4H1, 5C4, 4A11, 7D3, 5F4, and 2D3), WO2004/004771 and WO2004/056875 (including PD1-17, PD1-28, PD1-33, PD1-35, PD1-F2 and the Abs described in Table 1).

### Antibody-Drug Conjugates

Anti-ICOS antibodies can be used as carriers of cytotoxic agents, to target Tregs. As reported in Example 18 of WO2018/029474, Tregs located in the tumour microenvironment (TME) strongly express ICOS. ICOS is more strongly expressed on intratumoural Tregs than on intratumoural Teffs or peripheral Tregs. Thus, anti-ICOS antibodies labelled with a toxic drug or pro-drug may preferentially target Tregs in the TME to deliver the toxic payload, selectively inhibiting those cells. Such targeting of cytotoxic agents provides an additional route to removing the immune suppressive effect of Tregs, thereby altering the Treg:Teff balance in favour of Teff activity and may be used as an alternative to, or in combination with, any one or more of the other therapeutic approaches discussed herein (e.g., Fc effector-mediated inhibition of Tregs, agonism of effector T cells).

Accordingly, the invention provides an anti-ICOS antibody that is conjugated to a cytotoxic drug or pro-drug. In the case of a pro-drug, the pro-drug is activatable in the TME or other target site of therapeutic activity to generate the cytotoxic agent. Activation may be in response to a trigger such as photoactivation, e.g., using near-infrared light to activate a photoabsorber conjugate [35]. Spatially-selective activation of a pro-drug further enhances the cytotoxic effect of the antibody-drug conjugate, combining with the high ICOS expression on intratumoural Tregs to provide a cytotoxic effect that is highly selective for these cells.

For use in an antibody-drug conjugate, the cytotoxic drug or pro-drug is preferably non-immunogenic and non-toxic (dormant or inactive) during circulation of the antibody-drug conjugate in the blood. Preferably the cytotoxic drug (or the pro-drug, when activated) is potent—e.g., two to four molecules of the drug may be sufficient to kill the target cell. A photoactivatable pro-drug is silicapthalocyanine dye (IRDye 700 DX), which induces lethal damage to the cell membrane after near-infrared light exposure. Cytotoxic drugs include anti-mitotic agents such as monomethyl auristatin E and microtubule inhibitors such as maytansine derivatives, e.g., mertansine, DM1, emtansine.

Conjugation of the drug (or pro-drug) to the antibody will usually be via a linker. The linker may be a cleavable linker, e.g., disulphide, hydrazone or peptide link. Cathepsin-cleavable linkers may be used, so that the drug is released by cathepsin in tumour cells. Alternatively, non-cleavable linkers can be used, e.g., thioether linkage. Additional attachment groups and/or spacers may also be included.

The antibody in the antibody-drug conjugate may be an antibody fragment, such as Fab′2 or other antigen-binding fragment as described herein, as the small size of such fragments may assist penetration to the tissue site (e.g., solid tumour).

An anti-ICOS antibody according to the present invention may be provided as an immunocytokine. Anti-ICOS antibodies may also be administered with immunocytokines in combination therapy. A number of examples of antibodies are described herein for use in combination therapy with anti-ICOS, and any of these (e.g., an anti-PD-L1 antibody) may be provided as immunocytokines for use in the present invention. An immunocytokine comprises an antibody molecule conjugated to a cytokine, such as IL-2. Anti-ICOS:IL-2 conjugates and anti-PD-L1:IL-2 conjugates are thus further aspects of the present invention.

An IL-2 cytokine may have activity at the high (αβγ) affinity IL-2 receptor and/or the intermediate affinity (αβ) IL-2 receptor. IL-2 as used in an immunocytokine may be human wild type IL-2 or a variant IL-2 cytokine having one or more amino acid deletions, substitutions or additions, e.g., IL-2 having a 1 to 10 amino acid deletion at the N-terminus. Other IL-2 variants include mutations R38A or R38Q.

An example anti-PD-L1 immunocytokine comprises an immunoglobulin heavy chain and an immunoglobulin light chain, wherein the heavy chain comprises in N- to C-terminal direction:
a) A V_{H} domain comprising CDRH1, CDRH2 and CDRH3; and
b) A heavy chain constant region;
and wherein the light chain comprises in N- to C-terminal direction:
c) A V_{L} domain comprising CDRL1, CDRL2 and CDRL3;
d) A light chain constant region, (C_{L});
e) Optionally, a linker, (L); and
f) An IL-2 cytokine;
wherein the V_{H} domain and V_{L} domain are comprised by an antigen-binding site that specifically binds to human PD-L1; and
wherein the immunocytokine comprises a V_{H} domain which comprises a CDRH3 comprising the motif X₁GSGX₂YGX₃X₄FD (SEQ ID NO: 609), wherein X₁, X₂ and X₃ are independently any amino acid, and X₄ is either present or absent, and if present, may be any amino acid.

The VH and VL domain may be the VH and VL domain of any anti-PD-L1 antibody mentioned herein, e.g., the 1D05 VH and VL domains.

The IL-2 may be human wild type or variant IL-2.

### Vaccination

Anti-ICOS antibodies may be provided in vaccine compositions or co-administered with vaccines preparations. ICOS is involved in T follicular helper cell formation and the germinal centre reaction [36]. Agonist ICOS antibodies thus have potential clinical utility as molecular adjuvants to enhance vaccine efficacy. The antibodies may be used to increase protective efficacy of numerous vaccines, such as those against hepatitis B, malaria, HIV.

In the context of vaccination, the anti-ICOS antibody will generally be one that lacks Fc effector function, and thus does not mediate ADCC, CDC or ADCP. The antibody may be provided in a format lacking an Fc region, or having an effector null constant region. Optionally, an anti-ICOS antibody may have a heavy chain constant region that binds one or more types of Fc receptor but does not induce ADCC, CDC or ADCP activity, or that exhibits lower ADCC, CDC and ADCP activity compared with wild type human IgG1. Such a constant region may be unable to bind, or may bind with lower affinity, the particular Fc receptor(s) responsible for triggering ADCC, CDC or ADCP activity. Alternatively, where cellular effector functions are acceptable or desirable in the context of the vaccination, the anti-ICOS antibody may comprise a heavy chain constant region that is Fc effector function positive. Any of IgG1, IgG4 and IgG4.PE formats may for instance be used for anti-ICOS antibodies in vaccination regimens, and other examples of suitable isotypes and antibody constant regions are set out in more detail elsewhere herein.

### Formulations and Administration

Antibodies may be monoclonal or polyclonal, but are preferably provided as monoclonal antibodies for therapeutic use. They may be provided as part of a mixture of other antibodies, optionally including antibodies of different binding specificity.

Antibodies according to the invention, and encoding nucleic acid, will usually be provided in isolated form. Thus, the antibodies, VH and/or VL domains, and nucleic acids may be provided purified from their natural environment or their production environment. Isolated antibodies and isolated nucleic acid will be free or substantially free of material with which they are naturally associated, such as other polypeptides or nucleic acids with which they are found in vivo, or the environment in which they are prepared (e.g., cell culture) when such preparation is by recombinant DNA technology in vitro. Optionally an isolated antibody or nucleic acid (1) is free of at least some other proteins with which it would normally be found, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, or (6) does not occur in nature.

Antibodies or nucleic acids may be formulated with diluents or adjuvants and still for practical purposes be isolated—for example they may be mixed with carriers if used to coat microtitre plates for use in immunoassays, and may be mixed with pharmaceutically acceptable carriers or diluents when used in therapy. As described elsewhere herein, other active ingredients may also be included in therapeutic preparations. Antibodies may be glycosylated, either naturally in vivo or by systems of heterologous eukaryotic cells such as CHO cells, or they may be (for example if produced by expression in a prokaryotic cell) unglycosylated. The invention encompasses antibodies having a modified glycosylation pattern. In some applications, modification to remove undesirable glycosylation sites may be useful, or e.g., removal of a fucose moiety to increase ADCC function [37]. In other applications, modification of galactosylation can be made in order to modify CDC.

Typically, an isolated product constitutes at least about 5%, at least about 10%, at least about 25%, or at least about 50% of a given sample. An antibody may be substantially free from proteins or polypeptides or other contaminants that are found in its natural or production environment that would interfere with its therapeutic, diagnostic, prophylactic, research or other use.

An antibody may have been identified, separated and/or recovered from a component of its production environment (eg, naturally or recombinantly). The isolated antibody may be free of association with all other components from its production environment, eg, so that the antibody has been isolated to an FDA-approvable or approved standard. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In some embodiments, the antibody will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or silver stain. Isolated antibody includes the antibody in situ within recombinant cells since at least one component of the antibody's natural environment will not be present. Ordinarily, however, an isolated antibody or its encoding nucleic acid will be prepared by at least one purification step.

The invention provides therapeutic compositions comprising the antibodies described herein. Therapeutic compositions comprising nucleic acid encoding such antibodies are also provided. Encoding nucleic acids are described in more detail elsewhere herein and include DNA and RNA, e.g., mRNA. In therapeutic methods described herein, use of nucleic acid encoding the antibody, and/or of cells containing such nucleic acid, may be used as alternatives (or in addition) to compositions comprising the antibody itself. Cells containing nucleic acid encoding the antibody, optionally wherein the nucleic acid is stably integrated into the genome, thus represent medicaments for therapeutic use in a patient. Nucleic acid encoding the anti-ICOS antibody may be introduced into human B lymphocytes, optionally B lymphocytes derived from the intended patient and modified ex vivo. Optionally, memory B cells are used. Administration of cells containing the encoding nucleic acid to the patient provides a reservoir of cells capable of expressing the anti-ICOS antibody, which may provide therapeutic benefit over a longer term compared with administration of isolated nucleic acid or isolated antibody.

Compositions may contain suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTINT™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311. Compositions may comprise the antibody or nucleic acid in combination with medical injection buffer and/or with adjuvant.

Antibodies, or their encoding nucleic acids, may be formulated for the desired route of administration to a patient, e.g., in liquid (optionally aqueous solution) for injection. Various delivery systems are known and can be used to administer the pharmaceutical composition of the invention. Methods of introduction include, but are not limited to, intradermal, intramuscular, intraperitoneal, intravenous, subcutaneous, intranasal, epidural, and oral routes. Formulating antibodies for subcutaneous administration typically requires concentrating them into a smaller volume compared with intravenous preparations. The high potency of antibodies according to the present invention may lend them to use at sufficiently low doses to make subcutaneous formulation practical, representing an advantage compared with less potent anti-ICOS antibodies.

The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition can be also delivered in a vesicle, in particular a liposome (see Langer (1990) Science 249:1527-1533; Treat et al. (1989) in Liposomes in the Therapy of Infectious Disease and Cancer, Lopez Berestein and Fidler (eds.), Liss, New York, pp. 353-365; Lopez-Berestein, ibid., pp. 317-327; see generally ibid.).

In certain situations, the pharmaceutical composition can be delivered in a controlled release system. In one embodiment, a pump may be used (see Langer, supra; Sefton (1987) CRC Crit. Ref. Biomed. Eng. 14:201). In another embodiment, polymeric materials can be used; see, Medical Applications of Controlled Release, Langer and Wise (eds.), CRC Press., Boca Raton, Fla. (1974). In yet another embodiment, a controlled release system can be placed in proximity of the composition's target, thus requiring only a fraction of the systemic dose (see, e.g., Goodson, in Medical Applications of Controlled Release, supra, vol. 2, pp. 115-138, 1984).

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule. A pharmaceutical composition of the present invention can be delivered subcutaneously or intravenously with a standard needle and syringe. It is envisaged that treatment will not be restricted to use in the clinic. Therefore, subcutaneous injection using a needle-free device is also advantageous. With respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded. Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, Ind.), NOVOPEN™I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPENT™, OPTIPEN PRO™, OPTIPEN STARLET™, and OPTICLIKT™ (Sanofi-Aventis, Frankfurt, Germany), to name only a few. Examples of disposable pen delivery devices having applications in subcutaneous delivery of a pharmaceutical composition of the present invention include, but certainly are not limited to the SOLOSTAR™ pen (Sanofi-Aventis), the FLEXPEN™ (Novo Nordisk), and the KWIKPEN™ (Eli Lilly).

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, the aforesaid antibody may be contained in about 5 to about 100 mg and in about 10 to about 250 mg for the other dosage forms.

The antibody, nucleic acid, or composition comprising it, may be contained in a medical container such as a phial, syringe, IV container or an injection device. In an example, the antibody, nucleic acid or composition is in vitro, and may be in a sterile container. In an example, a kit is provided comprising the antibody, packaging and instructions for use in a therapeutic method as described herein.

One aspect of the invention is a composition comprising an antibody or nucleic acid of the invention and one or more pharmaceutically acceptable excipients, examples of which are listed above. “Pharmaceutically acceptable” refers to approved or approvable by a regulatory agency of the USA Federal or a state government or listed in the U.S. Pharmacopeia or other generally recognized pharmacopeia for use in animals, including humans. A pharmaceutically acceptable carrier, excipient, or adjuvant can be administered to a patient, together with an agent, e.g., any antibody or antibody chain described herein, and does not destroy the pharmacological activity thereof and is nontoxic when administered in doses sufficient to deliver a therapeutic amount of the agent.

In some embodiments, an anti-ICOS antibody will be the sole active ingredient in a composition according to the present invention. Thus, a composition may consist of the antibody or it may consist of the antibody with one or more pharmaceutically acceptable excipients. However, compositions according to the present invention optionally include one or more additional active ingredients. Detailed description of agents with which the anti-ICOS antibodies may be combined is provided elsewhere herein. Optionally, compositions contain multiple antibodies (or encoding nucleic acids) in a combined preparation, e.g., a single formulation comprising the anti-ICOS antibody and one or more other antibodies. Other therapeutic agents that it may be desirable to administer with antibodies or nucleic acids according to the present invention include analgaesic agents. Any such agent or combination of agents may be administered in combination with, or provided in compositions with antibodies or nucleic acids according to the present invention, whether as a combined or separate preparation. The antibody or nucleic acid according to the present invention may be administered separately and sequentially, or concurrently and optionally as a combined preparation, with another therapeutic agent or agents such as those mentioned.

Anti-ICOS antibodies for use in a particular therapeutic indication may be combined with the accepted standard of care. Thus, for anti-cancer treatment, the antibody therapy may be employed in a treatment regimen that also includes chemotherapy, surgery and/or radiation therapy for example. Radiotherapy may be single dose or in fractionated doses, either delivered to affected tissues directly or to the whole body.

Multiple compositions can be administered separately or simultaneously. Separate administration refers to the two compositions being administered at different times, e.g. at least 10, 20, 30, or 10-60 minutes apart, or 1, 2, 3, 4, 5, 6, 7, 8, 9, 10, 12 hours apart. One can also administer compositions at 24 hours apart, or even longer apart. Alternatively, two or more compositions can be administered simultaneously, e.g. less than 10 or less than 5 minutes apart. Compositions administered simultaneously can, in some aspects, be administered as a mixture, with or without similar or different time release mechanism for each of the components.

Antibodies, and their encoding nucleic acids, can be used as therapeutic agents. Patients herein are generally mammals, typically humans. An antibody or nucleic acid may be administered to a mammal, e.g., by any route of administration mentioned herein.

Administration is normally in a “therapeutically effective amount”, this being an amount that produces the desired effect for which it is administered, sufficient to show benefit to a patient. The exact amount will depend on the purpose of the treatment, and will be ascertainable by one skilled in the art using known techniques (see, for example, Lloyd (1999) The Art, Science and Technology of Pharmaceutical Compounding). Prescription of treatment, e.g. decisions on dosage etc, is within the responsibility of general practitioners and other medical doctors and may depend on the severity of the symptoms and/or progression of a disease being treated. A therapeutically effective amount or suitable dose of antibody or nucleic acid can be determined by comparing its in vitro activity and in vivo activity in an animal model. Methods for extrapolation of effective dosages in mice and other test animals to humans are known.

As indicated by the in vivo studies described in the Examples of WO2018/029474, anti-ICOS antibody may be effective at a range of doses. Pharmacodynamic studies are reported in Example 24 of WO2018/029474.

Anti-ICOS antibodies may be administered in an amount in one of the following ranges per dose:
about 10 μg/kg body weight to about 100 mg/kg body weight,
about 50 μg/kg body weight to about 5 mg/kg body weight,
about 100 μg/kg body weight to about 10 mg/kg body weight,
about 100 μg/kg body weight to about 20 mg/kg body weight,
about 0.5 mg/kg body weight to about 20 mg/kg body weight, or
about 5 mg/kg body weight or lower, for example less than 4, less than 3, less than 2, or less than 1 mg/kg of the antibody.

An optimal therapeutic dose may be between 0.1 and 0.5 mg/kg in a human, for example about 0.1 mg/kg, 0.15 mg/kg, 0.2 mg/kg, 0.25 mg/kg, 0.3 mg/kg, 0.35 mg/kg, 0.4 mg/kg, 0.45 mg/kg or 0.5 mg/kg. For fixed dosing in adult humans, a suitable dose may be between 8 and 50 mg, or between 8 and 25 mg, e.g., 15 mg or 20 mg.

In methods of treatment described herein, one or more doses may be administered. In some cases, a single dose may be effective to achieve a long-term benefit. Thus, the method may comprise administering a single dose of the antibody, its encoding nucleic acid, or the composition. Alternatively, multiple doses may be administered, usually sequentially and separated by a period of days, weeks or months. Anti-ICOS antibody may be repeatedly administered to a patient at intervals of 4 to 6 weeks, e.g., every 4 weeks, every 5 weeks, or every 6 weeks. Optionally, the anti-ICOS antibody may be administered to a patient once a month, or less frequently, e.g., every two months or every three months. Accordingly, a method of treating a patient may comprise administering a single dose of the anti-ICOS antibody to the patient, and not repeating the administration for at least one month, at least two months, at least three months, and optionally not repeating the administration for at least 12 months.

As discussed in Example 11c of WO2018/029474, comparable therapeutic effects may be obtained using either one or multiple doses of anti-ICOS antibody, which may be a result of a single dose of antibody being effective to reset the tumour microenvironment. Physicians can tailor the administration regimen of the anti-ICOS antibody to the disease and the patient undergoing therapy, taking into account the disease status and any other therapeutic agents or therapeutic measures (e.g., surgery, radiotherapy etc) with which the anti-ICOS antibody is being combined. In some embodiments, an effective dose of an anti-ICOS antibody is administered more frequently than once a month, such as, for example, once every three weeks, once every two weeks, or once every week. Treatment with anti-ICOS antibody may include multiple doses administered over a period of at least a month, at least six months, or at least a year.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilised (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment). For treatment to be effective a complete cure is not contemplated. The method can in certain aspects include cure as well. In the context of the invention, treatment may be preventative treatment.

### T Cell Therapy

WO2011/097477 described use of anti-ICOS antibodies for generating and expanding T cells, by contacting a population of T cells with a first agent that provides a primary activation signal (e.g., an anti-CD3 antibody) and a second agent that activates ICOS (e.g., an anti-ICOS antibody), optionally in the presence of a Th17 polarising agent such as IL-1β, IL-6, neutralising anti-IFNγ and/or anti-IL-4. Anti-ICOS antibodies described herein may be used in such methods to provide T cell populations. Populations of cultured expanded T cells having therapeutic activity (e.g., anti-tumour activity) may be generated. As described in WO2011/097477, such T cells may be used therapeutically in methods of treating patients by immunotherapy.

### Morphological Assay for Anti-ICOS Antibodies as Therapeutic Candidates

It was observed that when candidate therapeutic anti-ICOS antibodies were coupled to a solid surface and brought into contact with ICOS-expressing T cells, they were able to induce morphological change in the cells. On addition of ICOS+ T cells to wells that were internally coated with anti-ICOS antibodies, cells were seen to change from their initial rounded shape, adopting a spindle-shape, spreading and adhering to the antibody-coated surface. This morphological change was not observed with control antibody. Moreover, the effect was found to be dose-dependent, with faster and/or more pronounced shape change occurring as the concentration of antibody on the surface increased. The shape change provides a surrogate indicator of T cell binding to ICOS, and/or of agonism by anti-ICOS antibody. The assay may be used to identify an antibody that promotes multimerisation of ICOS on the T cell surface. Such antibodies represent therapeutic candidate agonist antibodies. Conveniently, the visual indicator provided by this assay is a simple method of screening antibodies or cells, particularly in large numbers. The assay may be automated to run in a high-throughput system.

Accordingly, one aspect of the invention is an assay for selecting an antibody that binds ICOS, optionally for selecting an ICOS agonist antibody, the assay comprising:
providing an array of antibodies immobilised (attached or adhered) to a substrate in a test well;
adding ICOS-expressing cells (e.g., activated primary T cells, or MJ cells) to the test well;
observing morphology of the cells;
detecting shape change in the cells from rounded to flattened against the substrate within the well; wherein the shape change indicates that the antibody is an antibody that binds ICOS, optionally an ICOS agonist antibody, and
selecting the antibody from the test well.

The assay may be run with multiple test wells, each containing a different antibody for testing, optionally in parallel, e.g., in a 96 well plate format. The substrate is preferably an inner surface of the well. Thus, a two-dimensional surface is provided against which flattening of the cells may be observed. For example, the bottom and/or wall of a well may be coated with antibody. Tethering of antibody to the substrate may be via a constant region of the antibody.

A negative control may be included, such an antibody known not to bind ICOS, preferably an antibody that does not bind an antigen on the surface of the ICOS-expressing cells to be used. The assay may comprise quantifying the degree of morphological change and, where multiple antibodies are tested, selecting an antibody that induces greater morphological change than one or more other test antibodies.

Selection of antibody may comprise expressing nucleic acid encoding the antibody present in the test well of interest, or expressing an antibody comprising the CDRs or antigen binding domain of that antibody. The antibody may optionally be reformatted, for example to provide an antibody comprising the antigen binding domain of the selected antibody, e.g., an antibody fragment, or an antibody comprising a different constant region. A selected antibody is preferably provided with a human IgG1 constant region or other constant region as described herein. A selected antibody may further be formulated in a composition comprising one or more additional ingredients—suitable pharmaceutical formations are discussed elsewhere herein.

Various further aspects and embodiments of the present invention will be apparent to those skilled in the art in view of the present disclosure. All documents mentioned in this specification, including published US counterparts of any patents or patent applications referred to, are incorporated herein by reference in their entirety.

### EXPERIMENTAL EXAMPLES

### Example 1—Study Background and Design

KY1044 (aka STIM003), is a fully human IgG1 anti ICOS (inducible T-cell co-stimulator) antibody designed to stimulate Teffs and to deplete ICOS high Tregs in the tumor microenvironment. ICOS is an important co-stimulatory receptor on effector T cells (Teffs) that also promotes tumor growth due to its high expression on regulatory T cells (Tregs). KY1044 is a fully human IgG1 that targets ICOS, acting via a dual mode of action (MoA) by depleting ICOS_{high} Tregs and stimulating ICOS_{Low} Teffs (Sainson R C A, Thotakura A K, Kosmac M, et al. An Antibody Targeting ICOS Increases Intratumoral Cytotoxic to Regulatory T-cell Ratio and Induces Tumor Regression. Cancer Immunology Research. 2020; 8(12):1568-1582)—see FIG. 1. KY1044-CT01 (ClinicalTrials.gov Identifier: NCT03829501) is a first-in-human study evaluating the safety, pharmacokinetics (PK), pharmacodynamics (PD) and preliminary antitumor activity of KY1044 as single agent and in combination with atezolizumab in patients with advanced/metastatic malignancies. Using longitudinal blood samples and tumor biopsies, we aim to correlate KY1044 target engagement levels with pharmacodynamic (PD) properties (e.g., dual MoA) in the tumor microenvironment (TME) and the circulation. The study consists of a Phase 1 dose escalation and enrichment cohorts and a Phase 2 part.

### Study Objectives

### Primary:

To characterize the safety and tolerability of KY1044 as single agent and in combination with atezolizumab and to identify recommended doses for future studies.

### Secondary:

To evaluate the preliminary anti tumor activity of KY1044 as single agent and in combination with atezolizumab.
To characterize the PK profile of KY1044 as single agent and in combination with atezolizumab.

### Exploratory:

To assess the pharmacodynamic effect of KY1044 as single agent and in combination with atezolizumab in tumor tissue and peripheral blood.

### Methods

### Key Inclusion Criteria:

Histologically documented advanced/metastatic malignancies who have measurable disease by RECIST 1.1 (non-measurable disease is allowed only in Phase 1)
Prior therapy with anti PD-(L)1 inhibitors is allowed provided any toxicity attributed to prior anti PD (L)1 directed therapy did not lead to discontinuation of therapy.
Eastern Cooperative Oncology Group performance status 0 or 1
Must have a site of disease amendable to biopsy, and be candidate for tumor biopsy, according to the treating institution's guidelines.

### Key Exclusion Criteria:

Symptomatic CNS metastases, or CNS metastases that require local CNS directed therapy.
Severe hypersensitivity reactions to other monoclonal antibodies or excipients.
Out-of-range laboratory values as defined in protocol for renal and hepatic function or hematology parameters.
Clinically significant heart disease and/or QT prolongation.
Active autoimmune disease or a documented history of autoimmune disease.
Systemic steroid therapy or any immunosuppressive therapy (≥10 mg/day prednisone or equivalent).
Presence of CTCAE v5≥Grade 2 toxicity due to prior anti-cancer therapy

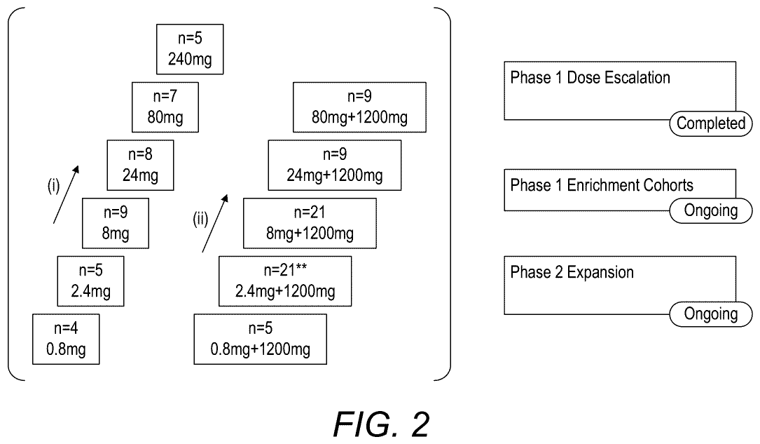

FIG. 2 outlines the study design.

### Results

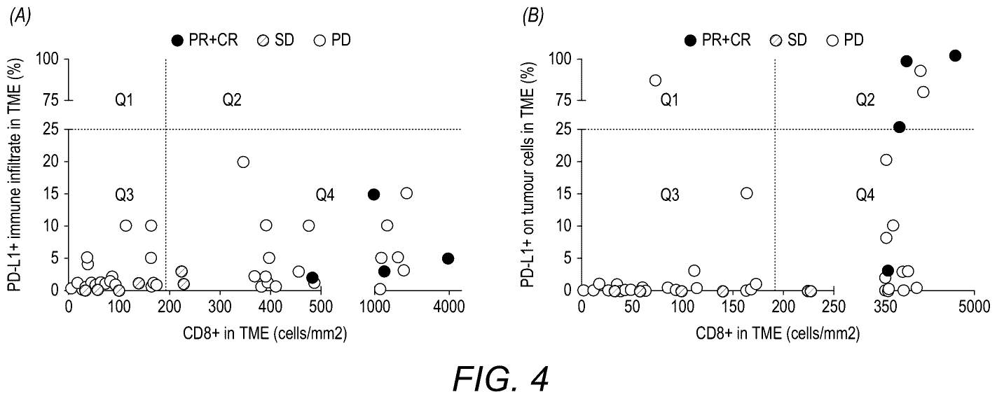

PD-L1 expression in the tumour microenvironment (TMW) was assessed on tumour and immune cells using the anti-PD-L1 antibody SP263 (see FIG. 3). FIG. 4 shows PD-L1 immune cell expression and CD8+ T cells in the TME (baseline).

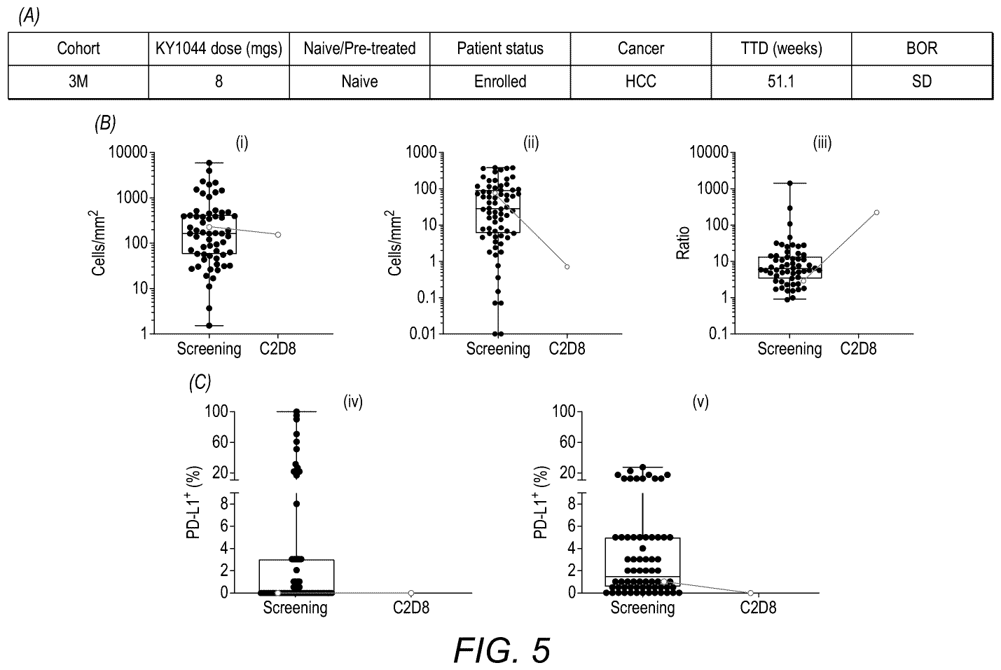

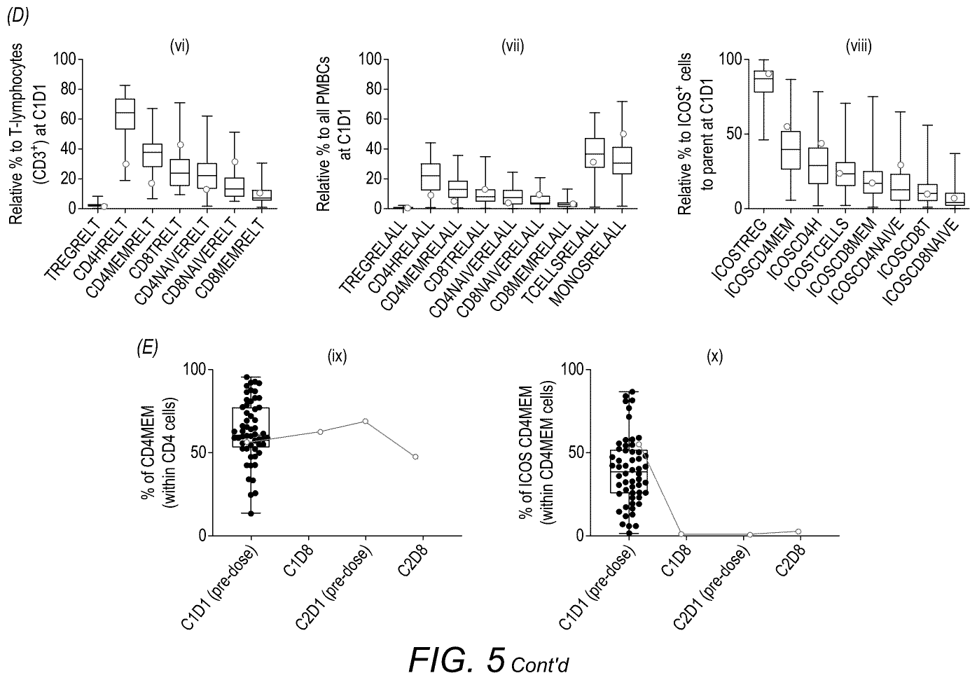

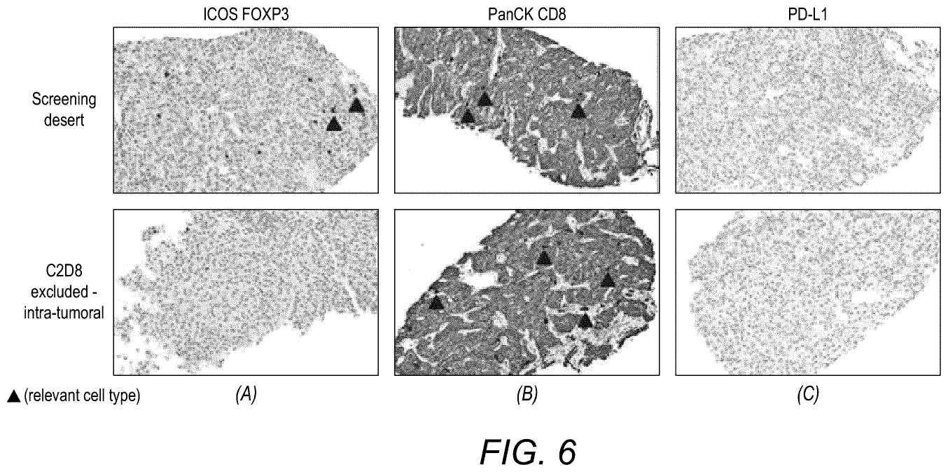

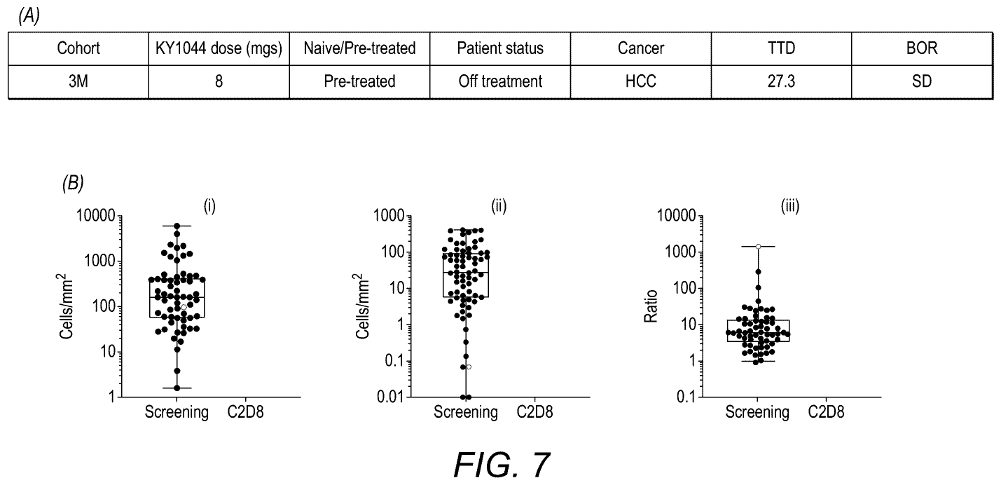

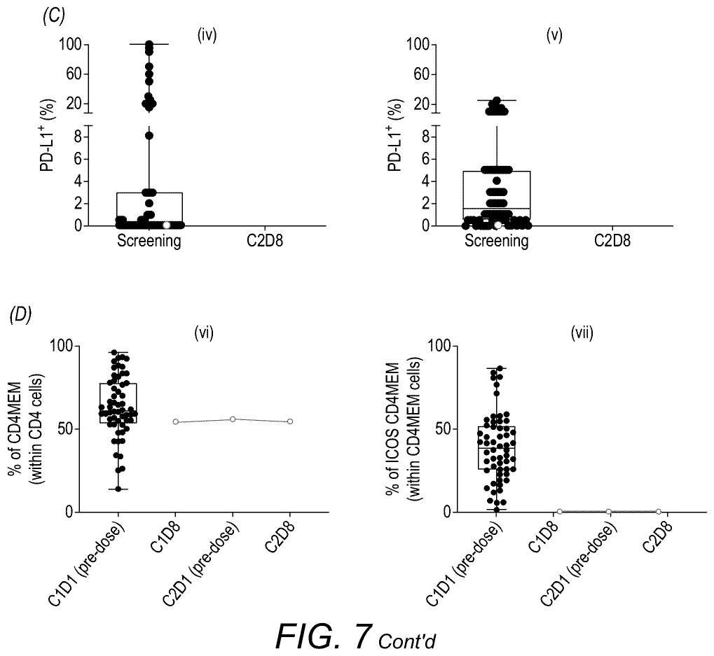

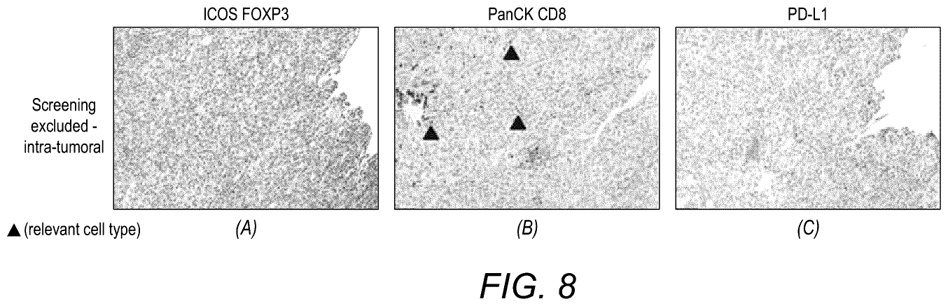

The effect of treatment on 3 patients (patients A, B and C) having PD-L1 negative tumours or PD-L1 low expression tumours was assessed. The results for Patient A are shown in FIGS. 5 and 6. The results for Patient B are shown in FIGS. 7 and 8. Despite having low PD-L1 expression, both patients arrived at a stable disease state (tumours neither growing nor shrinking) post treatment:

| | | PATIENT | CANCER | NAÏVE/ PRE- | | | | |
|---|---|---|---|---|---|---|---|---|
| Patient | COHORT | STATUS | TYPE | TREATED | EOT | TTD | STATUS | BOR |
| B | 3M | Off | HCC | Pre-treated | Discontinued | 27.3 | Ended | SD |
| | | treatment | | | | | | |
| A | 3M | Enrolled | HCC | Naive | | 55.1 | Ongoing | SD |

(SD = stable disease);
(BOR = best overall response);
(TTD = time to treatment discontinuation);
(EOT = end of test)

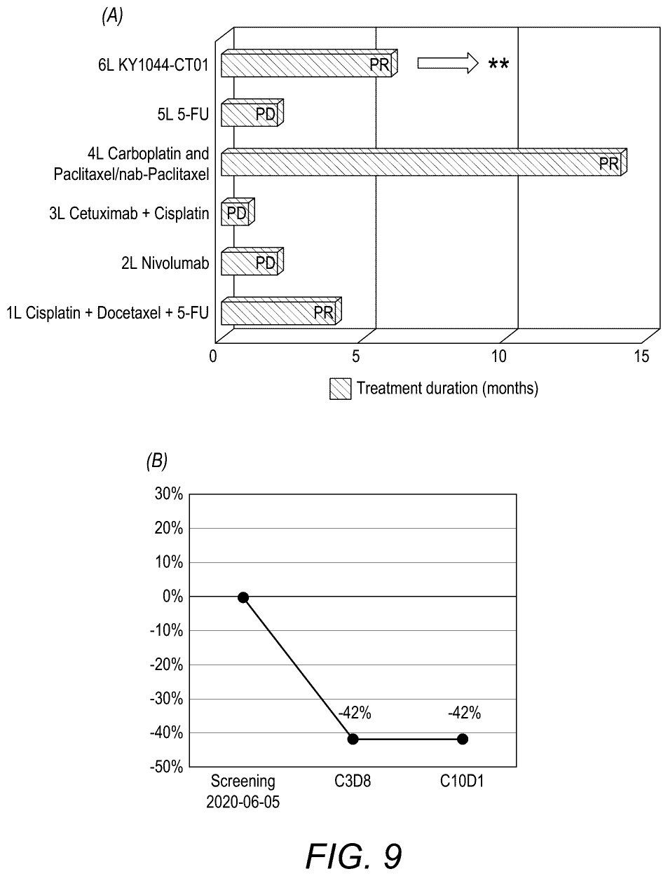

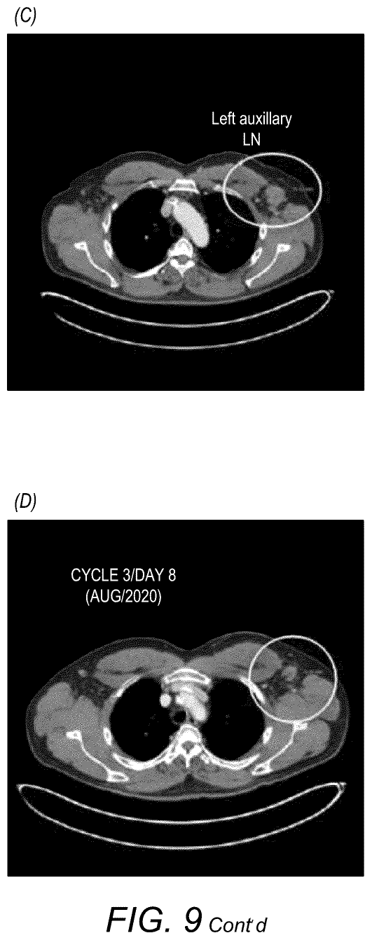

The results for Patient C are shown in FIG. 9. This patient responded well with a significant decrease in lesion size from baseline at C3D8 (3^{rd} cycle, day 8) and C10D1 (10^{th} cycle, day 1).

### HPV Status

Where the HPV status of the tumor was available, as part of the medical/tumor history of the patients enrolled, it was recorded.

A “HPV positive” tumor is deemed to be associated with or derived from HPV infection. A “HPV negative” tumor is deemed not to be associated with or derived from HPV infection.

Tests for the HPV status of a tumor are known in the art. Tests may include viral DNA detection, by polymerase chain reaction or in situ hybridization, or HPV RNA detection by reverse-transcription polymerase chain reaction or in situ hybridization. Tests for HPV status can be conducted on tissue biopsies, Fine-Needle Aspiration biopsy specimens, blood samples, or saliva samples, depending on the patient and the type of tumor.

The effect of treatment on 5 patients (patients D-H) was assessed. The results are shown below. Despite having low PD-L1 expression on tumor cells as well as on immune infiltrate, the patients reached partial response (PR). Moreover, the three patients (Patients E, G, and H) have been documented having a HPV positive tumor.

Without being bound by theory, the favourable outcome might be attributed to properties of HPV-positive tumor cells, such as, a decreased proliferation rate, or the patients having an improved immune response due to existing immune response directed towards the virus.

| | | | PD-L1+ on | PD-L1+ on |
|---|---|---|---|---|
| | BOR | | tumor cell | immune infiltrate |
| | (best | | in TME | cell in TME |
| | overall | HPV | (%) (as deter- | (%)(as deter- |
| Patient | response) | Status | mined by IHC) | mined by IHC) |
| D | PR | POSITIVE | 100 | 5 |
| E | PR | POSITIVE | 3 | 2 |
| F | PR | NEGATIVE | 0 | 3 |
| G* | PR | POSITIVE | 1 | 5 |
| H | PR | POSITIVE | 2 | 2 |

*Unconfirmed
(PR = partial response)

### CONCLUSIONS

The study concluded as follows:
Partial and transient receptor occupancy observed up to Dose Level 2 (2.4 mg) and complete and prolonged receptor occupancy at Dose Level 3 (8 mg) and above.
No significant depletion of ICOS+ T cells in the periphery.
KY1044 reduces ICOS+ Tregs and improves the ratio of CD8 to ICOS+ Tregs in the tumor microenvironment (Dose dependent, plateaued from dose level 3 [8 mg])
Sign of anti-tumor activity (PR/CR) observed in both PD-L1 low and PD-L1 high tumors.

### Example 2: Antibody Sequence Analysis

Framework regions of antibodies STIM001, STIM002, STIM002-B, STIM003, STIM004, STIM005, STIM006, STIM007, STIM008 and STIM009 were compared with human germline gene segments to identify the closest match. See Table E12-1 and Table E12-2.

TABLE E12-1
Heavy chain germline gene segments of anti-ICOS Abs

| Heavy chain | V | D | J |
|---|---|---|---|
| STIM001 | IGHV1-18*01 | IGHD6-19*01 | IGHJ6*02 |
| STIM002 | IGHV1-18*01 | IGHD3-10*01 | IGHJ6*02 |
| STIM002-B | IGHV1-18*01 | IGHD3-10*01 | IGHJ6*02 |
| STIM003 | IGHV3-20*d01 | IGHD3-10*01 | IGHJ4*02 |
| STIM004 | IGHV3-20*d01 | IGHD3-10*01 | IGHJ4*02 |
| STIM005 | IGHV1-18*01 | IGHD3-9*01 | IGHJ3*02 |
| STIM006 | IGHV3-11*01 | IGHD3-10*01 | IGHJ6*02 |
| STIM007 | IGHV2-5*10 | IGHD3-10*01 | IGHJ6*02 |
| STIM008 | IGHV2-5*10 | IGHD3-10*01 | IGHJ6*02 |
| STIM009 | IGHV3-11*01 | IGHD3-9*01 | IGHJ6*02 |

TABLE E12-2
Kappa light chain germline gene segments of anti-ICOS Abs

| Light chain | V | J |
|---|---|---|
| STIM001 | IGKV2-28*01 | IGKJ4*01 |
| STIM002 | IGKV2-28*01 | IGKJ2*04 |
| STIM002-B | IGKV2-28*01 | IGKJ2*04 |
| STIM003 | IGKV3-20*01 | IGKJ3*01 |
| STIM004 | IGKV3-20*01 | IGKJ3*01 |
| STIM005 | IGKV1D-39*01 | IGKJ1*01 |
| STIM006 | IGKV2-28*01 | IGKJ2*04 |
| STIM007 | IGKV3-11*01 | IGKJ4*01 |
| STIM008 | IGKV3-11*01 | IGKJ4*01 |
| STIM009 | IGKV2-28*01 | IGKJ1*01 |

Additional antibody sequences were obtained by next generation sequencing of PCR-amplified antibody DNA from further ICOS-specific cells that were sorted from the immunised mice as described in Example 3 of WO2018/029474. This identified a number of antibodies that could be grouped into clusters with STIM001, STIM002 or STIM003 based their heavy and light chain v and j gene segments and CDR3 length. CL-61091 clustered with STIM001; CL-64536, CL-64837, CL-64841 and CL-64912 clustered with STIM002; and CL-71642 and CL-74570 clustered with STIM003. Sequence alignments of the antibody VH and VL domains are shown in FIGS. 10 to 12.

TABLE E12-3
Antibodies clustered by sequence.

| ANTIBODIES | VH_V_GENE | VH_J_GENE | VH_CDR3_NT_LENGTH | VL_V_GENE | VL_J_GENE | VL_CDR3_NT_LENGTH |
|---|---|---|---|---|---|---|
| STIM001, CL-61091 | 1-18 | 6 | 42 | 2-28 | 4 | 27 |
| STIM002, CL-64536, | 1-18 | 6 | 51 | 2-28 | 2 | 27 |
| CL-64837, CL-64841, | | | | | | |
| CL-64912 | | | | | | |
| STIM003, CL-71642, | 3-20 | 4 | 51 | 3-20 | 3 | 27 |
| CL-74570 | | | | | | |
| STIM004 | 3-20 | 4 | 51 | 3-20 | 3 | 24 |
| STIM005 | 1-18 | 3 | 51 | 1D-39 | 1 | 24 |
| STIM006 | 3-11 | 6 | 63 | 2-28 | 2 | 30 |
| STIM007, STIM008 | 2-5 | 6 | 48 | 3-11 | 4 | 27 |
| STIM009 | 3-11 | 6 | 60 | 2-28 | 1 | 27 |

### REFERENCES

1 Hutloff A, et al. ICOS is an inducible T-cell co-stimulator structurally and functionally related to CD28. Nature. 1999 Jan. 21; 397(6716):263-6.
2 Beier K C, et al. Induction, binding specificity and function of human ICOS. Eur J Immunol. 2000 December; 30(12):3707-17.
3 Coyle A J, et al. The CD28-related molecule ICOS is required for effective T cell-dependent immune responses. Immunity. 2000 July; 13(1):95-105.
4 Dong C, et al. ICOS co-stimulatory receptor is essential for T-cell activation and function. Nature. 2001 Jan. 4; 409(6816):97-101.
5 Mak T W, et al. Costimulation through the inducible costimulator ligand is essential for both T helper and B cell functions in T cell-dependent B cell responses. Nat Immunol. 2003 August; 4(8):765-72.
6 Swallow M M, Wallin J J, Sha W C. B7h, a novel costimulatory homolog of B7.1 and B7.2, is induced by TNFalpha. Immunity. 1999 October; 11(4):423-32.
7 Wang S, et al. Costimulation of T cells by B7-H2, a B7-like molecule that binds ICOS. Blood. 2000 Oct. 15; 96(8):2808-13.
8 Conrad C, Gilliet M. Plasmacytoid dendritic cells and regulatory T cells in the tumor microenvironment: A dangerous liaison. Oncoimmunology. 2013 May 1; 2(5):e2388.
9 Simpson et al., Fc-dependent depletion of tumor-infiltrating regulatory T cells co-defines the efficacy of anti-CTLA-4 therapy against melanoma. J. Exp. Med. 210(9):1695-1710 2013
10 Fu T, He Q, Sharma P. The ICOS/ICOSL pathway is required for optimal antitumor responses mediated by anti-CTLA-4 therapy. Cancer Res. 2011 Aug. 15; 71(16):5445-54.
11 Fan X, Quezada S A, Sepulveda M A, Sharma P, Allison J P. Engagement of the ICOS pathway markedly enhances efficacy of CTLA-4 blockade in cancer immunotherapy. J Exp Med. 2014 Apr. 7; 211(4):715-25.
12 Carthon, B. C., et al. Preoperative CTLA-4 blockade: Tolerability and immune monitoring in the setting of a presurgical clinical trial. Clin. Cancer Res. 16:2861-2871.
13 Liakou Cl, et al. CTLA-4 blockade increases IFNgamma-producing CD4+ICOShi cells to shift the ratio of effector to regulatory T cells in cancer patients. Proc Natl Acad Sci USA. 2008 Sep. 30; 105(39):14987-92.
14 Vonderheide, R. H., et al. 2010. Tremelimumab in combination with exemestane in patients with advanced breast cancer and treatment-associated modulation of inducible costimulator expression on patient T cells. Clin. Cancer Res. 16:3485-3494.
15 Preston C C, et al., The ratios of CD8+ T cells to CD4+CD25+ FOXP3+ and FOXP3− T cells correlate with poor clinical outcome in human serous ovarian cancer. PLoS One November 14; 8(11):e80063.
16 Hodi F S, et al., Immunologic and clinical effects of antibody blockade of cytotoxic T lymphocyte-associated antigen 4 in previously vaccinated cancer patients. PNAS 2008 Feb. 26; 105(8):3005-10
17 Chattopadhyay et al., Structural Basis of Inducible Costimulatory Ligand Function: Determination of the Cell Surface Oligomeric State and Functional Mapping of the Receptor Binding Site of the Protein, J. Immunol. 177(6):3920-3929 2006
18 Lefranc M P, IMGT unique numbering for immunoglobulin and T cell receptor variable domains and Ig superfamily V-like domains, Dev Comp Immunol. 27(1):55-77 2003
19 Gül et al., “Antibody-Dependent Phagocytosis of Tumor Cells by Macrophages: A Potent Effector Mechanism of Monoclonal Antibody Therapy of Cancer”, Cancer Res., 75(23), Dec. 1, 2015
20 Lazar et al., 2006, Proc. Natl. Acad. Sci. U.S.A., March 14; 103(11):4005-10
21 Dall et al., Immunol 2002; 169:5171-5180
22 Natsume et al., 2009, Drug Des. Devel. Ther., 3:7-16 or by Zhou Q., Biotechnol. Bioeng., 2008, Feb. 15, 99(3):652-65)
23 Shields et al., 2001, J. Biol. Chem., March 2; 276(9):6591-604)
24 Idusogie et al., J. Immunol., 2001, 166:2571-2575 Natsume et al., 2008, Cancer Res., 68: 3863-3872
26 Alexandrov L B, et al. Signatures of mutational processes in human cancer. Nature. 2013 Aug. 22; 500(7463):415-21
27 Martin-Orozco et al., Melanoma Cells Express ICOS Ligand to Promote the Activation and Expansion of T-Regulatory Cells, Cancer Research 70(23):9581-9590 2010
28 Houot et al., Therapeutic effect of CD137 immunomodulation in lymphoma and its enhancement by Treg depletion, Blood 114:3431-3438 2009
29 Curran et al., PD01 and CTLA-4 combination blockade expands infiltrating T cells and reduces regulatory T and myeloid cells within B16 melanoma tumours, PNAS 107(9):4275-4280 2010
30 Sim et al., IL-2 therapy promotes suppressive ICOS+ Treg expansion in melanoma patients, J Clin Invest 2014
31 Sim et al., IL-2 variant circumvents ICOS+ regulatory T cell expansion and promotes NK cell activation, Cancer Immunol Res 2016
32 Kroemer et al. Immunologic Cell Death in Cancer Therapy, Ann Rev Immunol. 31:51-72 2013
33 Galluzzi, Zitvogel & Kroemer Canc. Imm. Res. 4:895-902 2016
34 Bos et al., Transient regulatory T cell ablation deters oncogene-driven breast cancer and enhances radiotherapy, J Exp Med 210(11):2434-2446 2013
35 Sato et al., Spatially selective depletion of tumor-associated regulatory T cells with near-infrared photoimmunotherapy, Science Translational Medicine 8(352) 2016
37 Crotty S. T follicular helper cell differentiation, function, and roles in disease. Immunity. 2014 Oct. 16; 41(4):529-42.
37 Shields et al. (2002) JBC 277:26733

Sequences
Antibody STIM001
VH domain nucleotide sequence: SEQ ID NO: 367
CAGGTTCAGGTGGTGCAGTCTGGAGCTGAGGTGAAGAAGCCTGGGGCCTCAGTGAAGGTCTCCTGCAAGGCTTCTGG
TTACACCTTTTCCACCTTTGGTATCACCTGGGTGCGACAGGCCCCTGGACAAGGGCTTGAATGGATGGGATGGATCA
GCGCTTACAATGGTGACACAAACTATGCACAGAATCTCCAGGGCAGAGTCATCATGACCACAGACACATCCACGAGC
ACAGCCTACATGGAGCTGAGGAGCCTGAGATCTGACGACACGGCCGTTTATTACTGTGCGAGGAGCAGTGGCCACTA
CTACTACTACGGTATGGACGTCTGGGGCCAAGGGACCACGGTCACCGTCTCCTCA
VH domain amino acid sequence: SEQ ID NO: 366
QVQVVQSGAEVKKPGASVKVSCKASGYTFSTFGITWVRQAPGQGLEWMGWISAYNGDTNYAQNLQGRVIMTTDTSTS
TAYMELRSLRSDDTAVYYCARSSGHYYYYGMDVWGQGTTVTVSS
VH CDR1 amino acid sequence: GYTFSTFG SEQ ID NO: 363
VH CDR2 amino acid sequence: ISAYNGDT SEQ ID NO: 364
VH CDR3 amino acid sequence: ARSSGHYYYYGMDV SEQ ID NO: 365
VL domain nucleotide sequence: SEQ ID NO: 374
GATATTGTGATGACTCAGTCTCCACTCTCCCTGCCCGTCACCCCTGGAGAGCCGGCCTCCATCTCCTGCAGGTCTAG
TCAGAGCCTCCTGCATAGTAATGAATACAACTATTTGGATTGGTACCTGCAGAAGCCAGGGCAGTCTCCACAGCTCC
TGATCTTTTTGGGTTCTAATCGGGCCTCCGGGGTCCCTGACAGGTTCAGTGGCAGTGGATCAGGCACAGATTTTACA
CTGAAAATCACCAGAGTGGAGGCTGAGGATGTTGGAATTTATTACTGCATGCAATCTCTACAAACTCCGCTCACTTT
CGGCGGAGGGACCAAGGTGGAGATCAAA
VL domain amino acid sequence: SEQ ID NO: 373
DIVMTQSPLSLPVTPGEPASISCRSSQSLLHSNEYNYLDWYLQKPGQSPQLLIFLGSNRASGVPDRFSGSGSGTDFT
LKITRVEAEDVGIYYCMQSLQTPLTFGGGTKVEIK
VL CDR1 amino acid sequence: QSLLHSNEYNY SEQ ID NO: 370
VL CDR2 amino acid sequence: LGS SEQ ID NO: 371
VL CDR3 amino acid sequence: MQSLQTPLT SEQ ID NO: 372
Antibody STIM002
VH domain nucleotide sequence: SEQ ID NO: 381
CAGGTTCAACTGGTGCAGTCTGGAGGTGAGGTGAAGAAGCCTGGGGCCTCAGTGAAGGTCTCCTGCAAGGCTTCTGG
TTACACCTTTACCAGCTATGGTTTCAGCTGGGTGCGACAGGCCCCTGGACAAGGACTAGAGTGGATGGGATGGATCA
GCGCTTACAATGGTAACACAAACTATGCACAGAAGCTCCAGGGCAGAGTCACCATGACCACAGACACATCCACGAGC
ACAGCCTACATGGAGCTGAGGAGCTTGAGATCTGACGACACGGCCGTGTATTACTGTGCGAGATCTACGTATTTCTA
TGGTTCGGGGACCCTCTACGGTATGGACGTCTGGGGCCAAGGGACCACGGTCACCGTCTCCTCA
VH domain amino acid sequence: SEQ ID NO: 380
QVQLVQSGGEVKKPGASVKVSCKASGYTFTSYGFSWVRQAPGQGLEWMGWISAYNGNTNYAQKLQGRVTMTTDTSTS
TAYMELRSLRSDDTAVYYCARSTYFYGSGTLYGMDVWGQGTTVTVSS
VH CDR1 amino acid sequence: GYTFTSYG SEQ ID NO: 377
VH CDR2 amino acid sequence: ISAYNGNT SEQ ID NO: 378
VH CDR3 amino acid sequence: ARSTYFYGSGTLYGMDV SEQ ID NO: 379
VL domain nucleotide sequence: 388
GATATTGTGATGACTCAGTCTCCACTCTCCCTGCCCGTCACCCCTGGAGAGCCGGCCTCCATCTCCTGCAGGTCTAG
TCAGAGCCTCCTGCATAGTGATGGATACAACTGTTTGGATTGGTACCTGCAGAAGCCAGGGCAGTCTCCACAGCTCC
TGATCTATTTGGGTTCTACTCGGGCCTCCGGGTTCCCTGACAGGTTCAGTGGCAGTGGATCAGGCACAGATTTTACA
CTGAAAATCAGCAGAGTGGAGGCTGAGGATGTTGGGGTTTATTACTGCATGCAAGCTCTACAAACTCCGTGCAGTTT
TGGCCAGGGGACCAAGCTGGAGATCAAA
Corrected STIM002 VL domain nucleotide sequence: SEQ ID NO: 519
GATATTGTGATGACTCAGTCTCCACTCTCCCTGCCCGTCACCCCTGGAGAGCCGGCCTCCATCTCCTGCAGGTCTAG
TCAGAGCCTCCTGCATAGTGATGGATACAACTATTTGGATTGGTACCTGCAGAAGCCAGGGCAGTCTCCACAGCTCC
TGATCTATTTGGGTTCTACTCGGGCCTCCGGGTTCCCTGACAGGTTCAGTGGCAGTGGATCAGGCACAGATTTTACA
CTGAAAATCAGCAGAGTGGAGGCTGAGGATGTTGGGGTTTATTACTGCATGCAAGCTCTACAAACTCCGCTCAGTTT
TGGCCAGGGGACCAAGCTGGAGATCAAA
VL domain amino acid sequence: SEQ ID NO: 387
DIVMTQSPLSLPVTPGEPASISCRSSQSLLHSDGYNYLDWYLQKPGQSPQLLIYLGSTRASGFPDRFSGSGSGTDFT
LKISRVEAEDVGVYYCMQALQTPLSFGQGTKLEIK
VL CDR1 amino acid sequence: QSLLHSDGYNY SEQ ID NO: 384
VL CDR2 amino acid sequence: LGS SEQ ID NO: 385
VL CDR3 amino acid sequence: MQALQTPLS SEQ ID NO: 386
Antibody STIM002-B
VH domain nucleotide sequence: SEQ ID NO: 395
CAGGTTCAACTGGTGCAGTCTGGAGGTGAGGTGAAGAAGCCTGGGGCCTCAGTGAAGGTCTCCTGCAAGGCTTCTGG
TTACACCTTTACCAGCTATGGTTTCAGCTGGGTGCGACAGGCCCCTGGACAAGGACTAGAGTGGATGGGATGGATCA
GCGCTTACAATGGTAACACAAACTATGCACAGAAGCTCCAGGGCAGAGTCACCATGACCACAGACACATCCACGAGC
ACAGCCTACATGGAGCTGAGGAGCTTGAGATCTGACGACACGGCCGTGTATTACTGTGCGAGATCTACGTATTTCTA
TGGTTCGGGGACCCTCTACGGTATGGACGTCTGGGGCCAAGGGACCACGGTCACCGTCTCCTCA
VH domain amino acid sequence: SEQ ID NO: 394
QVQLVQSGGEVKKPGASVKVSCKASGYTFTSYGFSWVRQAPGQGLEWMGWISAYNGNTNYAQKLQGRVTMTTDTSTS
TAYMELRSLRSDDTAVYYCARSTYFYGSGTLYGMDVWGQGTTVTVSS
VH CDR1 amino acid sequence: GYTFTSYG SEQ ID NO: 391
VH CDR2 amino acid sequence: ISAYNGNT SEQ ID NO: 392
VH CDR3 amino acid sequence: ARSTYFYGSGTLYGMDV SEQ ID NO: 393
VL domain nucleotide sequence: SEQ ID NO: 402
GATATTGTGATGACTCAGTCTCCACTCTCCCTGCCCGTCACCCCTGGAGAGCCGGCCTCCATCTCCTGCAGGTCTAG
TCAGAGCCTCCTGCATAGTGATGGATACAACTGTTTGGATTGGTACCTGCAGAAGCCAGGGCAGTCTCCACAGCTCC
TGATCTATTTGGGTTCTACTCGGGCCTCCGGGTTCCCTGACAGGTTCAGTGGCAGTGGATCAGGCACAGATTTTACA
CTGAAAATCAGCAGAGTGGAGGCTGAGGATGTTGGGGTTTATTACTGCATGCAAGCTCTACAAACTCCGTGCAGTTT
TGGCCAGGGGACCAAGCTGGAGATCAAA
VL domain amino acid sequence: SEQ ID NO: 401
DIVMTQSPLSLPVTPGEPASISCRSSQSLLHSDGYNCLDWYLQKPGQSPQLLIYLGSTRASGFPDRFSGSGSGTDET
LKISRVEAEDVGVYYCMQALQTPCSFGQGTKLEIK
VL CDR1 amino acid sequence: QSLLHSDGYNC SEQ ID NO: 398
VL CDR2 amino acid sequence: LGS SEQ ID NO: 399
VL CDR3 amino acid sequence: MQALQTPCS SEQ ID NO: 400
Antibody STIM003
VH domain nucleotide sequence: SEQ ID NO: 409
GAGGTGCAGCTGGTGGAGTCTGGGGGAGGTGTGGTACGGCCTGGGGGGTCCCTGAGACTCTCCTGTGTAGCCTCTGG
AGTCACCTTTGATGATTATGGCATGAGCTGGGTCCGCCAAGCTCCAGGGAAGGGGCTGGARTGGGTCTCTGGTATTA
ATTGGAATGGTGGCGACACAGATTATTCAGACTCTGTGAAGGGCCGATTCACCATCTCCAGAGACAACGCCAAGAAC
TCCCTGTATCTACAAATGAATAGTCTGAGAGCCGAGGACACGGCCTTGTATTACTGTGCGAGGGATTTCTATGGTTC
GGGGAGTTATTATCACGTTCCTTTTGACTACTGGGGCCAGGGAATCCTGGTCACCGTCTCCTCA
Corrected STIM003 VH domain nucleotide sequence: SEQ ID NO: 521
GAGGTGCAGCTGGTGGAGTCTGGGGGAGGTGTGGTACGGCCTGGGGGGTCCCTGAGACTCTCCTGTGTAGCCTCTGG
AGTCACCTTTGATGATTATGGCATGAGCTGGGTCCGCCAAGCTCCAGGGAAGGGGCTGGAGTGGGTCTCTGGTATTA
ATTGGAATGGTGGCGACACAGATTATTCAGACTCTGTGAAGGGCCGATTCACCATCTCCAGAGACAACGCCAAGAAC
TCCCTGTATCTACAAATGAATAGTCTGAGAGCCGAGGACACGGCCTTGTATTACTGTGCGAGGGATTTCTATGGTTC
GGGGAGTTATTATCACGTTCCTTTTGACTACTGGGGCCAGGGAATCCTGGTCACCGTCTCCTCA
VH domain amino acid sequence: SEQ ID NO: 408
EVQLVESGGGVVRPGGSLRLSCVASGVTFDDYGMSWVRQAPGKGLEWVSGINWNGGDTDYSDSVKGRFTISRDNAKN
SLYLQMNSLRAEDTALYYCARDFYGSGSYYHVPFDYWGQGILVTVSS
VH CDR1 amino acid sequence: GVTFDDYG SEQ ID NO: 405
VH CDR2 amino acid sequence: INWNGGDT SEQ ID NO: 406
VH CDR3 amino acid sequence: ARDFYGSGSYYHVPFDY SEQ ID NO: 407
VL domain nucleotide sequence: SEQ ID NO: 416
GAAATTGTGTTGACGCAGTCTCCAGGGACCCTGTCTTTGTCTCCAGGGGAAAGAGCCACCCTCTCCTGCAGGGCCAG
TCAGAGTGTTAGCAGAAGCTACTTAGCCTGGTACCAGCAGAAACGTGGCCAGGCTCCCAGGCTCCTCATCTATGGTG
CATCCAGCAGGGCCACTGGCATCCCAGACAGGTTCAGTGGCGATGGGTCTGGGACAGACTTCACTCTCTCCATCAGC
AGACTGGAGCCTGAAGATTTTGCAGTGTATTACTGTCACCAGTATGATATGTCACCATTCACTTTCGGCCCTGGGAC
CAAAGTGGATATCAAA
VL domain amino acid sequence: SEQ ID NO: 415
EIVLTQSPGTLSLSPGERATLSCRASQSVSRSYLAWYQQKRGQAPRLLIYGASSRATGIPDRFSGDGSGTDFTLSIS
RLEPEDFAVYYCHQYDMSPFTFGPGTKVDIK
VL CDR1 amino acid sequence: QSVSRSY SEQ ID NO: 412
VL CDR2 amino acid sequence: GAS SEQ ID NO: 413
VL CDR3 amino acid sequence: HQYDMSPFT SEQ ID NO: 414
Antibody STIM004
VH domain nucleotide sequence:
GAGGTGCAGCTGGTGGAGTCTGGGGGAGGTGTGGTACGGCCTGGGGGGTCCCTGAGACTCTCCTGTGCAGCCTCTGG
ACTCACCTTTGATGATTATGGCATGAGCTGGGTCCGCCAAGTTCCAGGGAAGGGGCTGGAGTGGGTCTCTGGTATTA
ATTGGAATGGTGATAACACAGATTATGCAGACTCTGTGAAGGGCCGATTCACCATCTCCAGAGACAACGCCAAGAAC
TCCCTGTATCTGCAAATGAACAGTCTGAGAGCCGAGGACACGGCCTTGTATTACTGTGCGAGGGATTACTATGGTTC
GGGGAGTTATTATAACGTTCCTTTTGACTACTGGGGCCAGGGAACCCTGGTCACCGTCTCCTCA SEQ ID NO: 423
VH domain amino acid sequence:
EVQLVESGGGVVRPGGSLRLSCAASGLTFDDYGMSWVRQVPGKGLEWVSGINWNGDNTDYADSVKGRFTISRDNAKN
SLYLQMNSLRAEDTALYYCARDYYGSGSYYNVPFDYWGQGTLVTVSS SEQ ID NO: 422
VH CDR1 amino acid sequence: GLTFDDYG SEQ ID NO: 419
VH CDR2 amino acid sequence: INWNGDNT SEQ ID NO: 420
VH CDR3 amino acid sequence: ARDYYGSGSYYNVPFDY SEQ ID NO: 421
VL domain nucleotide sequence: SEQ ID NO: 431
GAAATTGTGTTGACGCAGTCTCCAGGCACCCTGTCTTTGTCTCCAGGGGAAAGAGCCACCCTCTCCTGCAGGGCCAG
TCAGAGTGTTAGCAGCAGCTACTTAGCCTGGTACCAGCAGAAACCTGGCCAGGCTCCCAGGCTCCTCATATATGGTG
CATCCAGCAGGGCCACTGGCATCCCAGACAGGTTCAGTGGCAGTGGGTCTGGGACAGACTTCACTCTCACCATCAGA
AGACTGGAGCCTGAAGATTTTGCAGTGTATTACTGTCAGCAGTATGGTAGTTCACCATTCACTTCGGCCCTGGGACC
AAAGTGGATATCAAA
VL domain amino acid sequence as encoded by the above VL domain nucleotide
sequence.
Corrected VL domain nucleotide sequence: SEQ ID NO: 430
GAAATTGTGTTGACGCAGTCTCCAGGCACCCTGTCTTTGTCTCCAGGGGAAAGAGCCACCCTCTCCTGCAGGGCCAG
TCAGAGTGTTAGCAGCAGCTACTTAGCCTGGTACCAGCAGAAACCTGGCCAGGCTCCCAGGCTCCTCATATATGGTG
CATCCAGCAGGGCCACTGGCATCCCAGACAGGTTCAGTGGCAGTGGGTCTGGGACAGACTTCACTCTCACCATCAGA
AGACTGGAGCCTGAAGATTTTGCAGTGTATTACTGTCAGCAGTATGGTAGTTCACCATTCTTCGGCCCTGGGACCAA
AGTGGATATCAAA
Corrected VL domain amino acid sequence: SEQ ID NO: 432
EIVLTQSPGTLSLSPGERATLSCRASQSVSSSYLAWYQQKPGQAPRLLIYGASSRATGIPDRFSGSGSGTDFTLTIR
RLEPEDFAVYYCQQYGSSPFFGPGTKVDIK
VL CDR1 amino acid sequence: QSVSSSY SEQ ID NO: 426
VL CDR2 amino acid sequence: GAS SEQ ID NO: 427
VL CDR3 amino acid sequence: QQYGSSPF SEQ ID NO: 428
Antibody STIM005
VH domain nucleotide sequence: SEQ ID NO: 439
CAGGTTCAGTTGGTGCAGTCTGGAGCTGAGGTGAAGAAGCCTGGGGCCTCAGTGAAGGTCTCCTGCAAGGCTTCTGG
TTACACCTTTAATAGTTATGGTATCATCTGGGTGCGACAGGCCCCTGGACAAGGGCTTGAGTGGATGGGATGGATCA
GCGTTCACAATGGTAACACAAACTGTGCACAGAAGCTCCAGGGTAGAGTCACCATGACCACAGACACATCCACGAGC
ACAGCCTACATGGAGCTGAGGAGCCTGAGAACTGACGACACGGCCGTGTATTACTGTGCGAGAGCGGGTTACGATAT
TTTGACTGATTTTTCCGATGCTTTTGATATCTGGGGCCACGGGACAATGGTCACCGTCTCTTCA
VH domain amino acid sequence: SEQ ID NO: 438
QVQLVQSGAEVKKPGASVKVSCKASGYTFNSYGIIWVRQAPGQGLEWMGWISVHNGNTNCAQKLQGRVTMTTDTSTS
TAYMELRSLRTDDTAVYYCARAGYDILTDFSDAFDIWGHGTMVTVSS
VH CDR1 amino acid sequence: GYTFNSYG SEQ ID NO: 435
VH CDR2 amino acid sequence: ISVHNGNT SEQ ID NO: 436
VH CDR3 amino acid sequence: ARAGYDILTDESDAFDI SEQ ID NO: 437
VL domain nucleotide sequence: SEQ ID NO: 446
GACATCCAGATGACCCAGTCTCCATCCTCCCTGTCTGCATCTGTAGGAGACAGAGTCACCATCACTTGCCGGGCAAG
TCAGAACATTAATAACTTTTTAAATTGGTATCAGCAGAAAGAAGGGAAAGGCCCTAAGCTCCTGATCTATGCAGCAT
CCAGTTTGCAAAGAGGGATACCATCAACGTTCAGTGGCAGTGGATCTGGGACAGACTTCACTCTCACCATCAGCAGT
CTGCAACCTGAAGATTTTGCAACTTACATCTGTCAACAGAGCTACGGTATCCCGTGGGTCGGCCAAGGGACCAAGGT
GGAAATCAAA
VL domain amino acid sequence: SEQ ID NO: 445
DIQMTQSPSSLSASVGDRVTITCRASQNINNFLNWYQQKEGKGPKLLIYAASSLQRGIPSTFSGSGSGTDFTLTISS
LQPEDFATYICQQSYGIPWVGQGTKVEIK
VL CDR1 amino acid sequence: QNINNF SEQ ID NO: 442
VL CDR2 amino acid sequence: AAS SEQ ID NO: 443
VL CDR3 amino acid sequence: QQSYGIPW SEQ ID NO: 444
Antibody STIM006
VH domain nucleotide sequence: SEQ ID NO: 453
CAGGTGCAGCTGGTGGAGTCTGGGGGAGGCTTGGTCAAGCCTGGAGGGTCCCTGAGACTCTCCTGTGCAGCCTCTGG
ATTCACCTTCAGTGACTACTTCATGAGCTGGATCCGCCAGGCGCCAGGGAAGGGGCTGGAGTGGATTTCATACATTA
GTTCTAGTGGTAGTACCATATACTACGCAGACTCTGTGAGGGGCCGATTCACCATCTCCAGGGACAACGCCAAGTAC
TCACTGTATCTGCAAATGAACAGCCTGAGATCCGAGGACACGGCCGTGTATTACTGTGCGAGAGATCACTACGATGG
TTCGGGGATTTATCCCCTCTACTACTATTACGGTTTGGACGTCTGGGGCCAGGGGACCACGGTCACCGTCTCCTCA
VH domain amino acid sequence: SEQ ID NO: 454
QVQLVESGGGLVKPGGSLRLSCAASGFTFSDYFMSWIRQAPGKGLEWISYISSSGSTIYYADSVRGRFTISRDNAKY
SLYLQMNSLRSEDTAVYYCARDHYDGSGIYPLYYYYGLDVWGQGTTVTVSS
VH CDR1 amino acid sequence: GFTFSDYF SEQ ID NO: 449
VH CDR2 amino acid sequence: ISSSGSTI SEQ ID NO: 450
VH CDR3 amino acid sequence: ARDHYDGSGIYPLYYYYGLDV SEQ ID NO: 451
VL domain nucleotide sequence: SEQ ID NO: 460
ATTGTGATGACTCAGTCTCCACTCTCCCTACCCGTCACCCCTGGAGAGCCGGCCTCCATCTCCTGCAGGTCTAGTCA
GAGCCTCCTGCATAGTAATGGATACAACTATTTGGATTATTACCTGCAGAAGCCAGGGCAGTCTCCACAGCTCCTGA
TCTATTTGGGTTCTTATCGGGCCTCCGGGGTCCCTGACAGGTTCAGTGGCAGTGGATCAGGCACAGATTTTACACTG
AAAATCAGCAGAGTGGAGGCTGAGGATGTTGGGGTTTATTACTGCATGCAAGCTCTACAAACTCCTCGCAGTTTTGG
CCAGGGGACCACGCTGGAGATCAAA
VL domain amino acid sequence: SEQ ID NO: 459
IVMTQSPLSLPVTPGEPASISCRSSQSLLHSNGYNYLDYYLQKPGQSPQLLIYLGSYRASGVPDRFSGSGSGTDFTL
KISRVEAEDVGVYYCMQALQTPRSFGQGTTLEIK
VL CDR1 amino acid sequence: QSLLHSNGYNY SEQ ID NO: 456
VL CDR2 amino acid sequence: LGS SEQ ID NO: 457
VL CDR3 amino acid sequence: MQALQTPRS SEQ ID NO: 458
Antibody STIM007
VH domain nucleotide sequence: SEQ ID NO: 467
CAGATCACCTTGAAGGAGTCTGGTCCTACGCTGGTGAAACCCACACAGACCCTCACGCTGACCTGCACCTTCTCTGG
GTTCTCACTCAGCACTACTGGAGTGGGTGTGGGCTGGATCCGTCAGCCCCCAGGAAAGGCCCTGGAGTGGCTTGCAG
TCATTTATTGGGATGATGATAAGCGCTACAGCCCATCTCTGAAGAGCAGACTCACCATCACCAAGGACACCTCCAAA
AACCAGGTGGTCCTTACAATGACCAACATGGACCCTGTGGACACAGCCACATATTTCTGTACACACGGATATGGTTC
GGCGAGTTATTACCACTACGGTATGGACGTCTGGGGCCAAGGGACCACGGTCACCGTCTCCTCA
VH domain amino acid sequence: SEQ ID NO: 466
QITLKESGPTLVKPTQTLTLTCTFSGFSLSTTGVGVGWIRQPPGKALEWLAVIYWDDDKRYSPSLKSRLTITKDTSK
NQVVLTMTNMDPVDTATYFCTHGYGSASYYHYGMDVWGQGTTVTVSS
VH CDR1 amino acid sequence: GFSLSTTGVG SEQ ID NO: 463
VH CDR2 amino acid sequence: IYWDDDK SEQ ID NO: 464
VH CDR3 amino acid sequence: THGYGSASYYHYGMDV SEQ ID NO: 465
VL domain nucleotide sequence: SEQ ID NO: 474
GAAATTGTATTGACACAGTCTCCAGCCACCCTGTCTTTGTCTCCAGGGGAAAGAGCCACCCTCTCCTGCAGGGCCAG
TCAGAGTGTTACCAACTACTTAGCCTGGCACCAACAGAAACCTGGCCAGGCTCCCAGGCTCCTCATCTATGATGCAT
CCAACAGGGCCACTGGCATCCCAGCCAGGTTCAGTGGCAGTGGGTCTGGGACAGACTTCACTCTCACCATCAGCAGC
CTAGAGCCTGAAGATTTTGCAGTTTATTACTGTCAGCACCGTAGCAACTGGCCTCTCACTTTCGGCGGAGGGACCAA
GGTGGAGATCAAAC
VL domain amino acid sequence: SEQ ID NO: 473
EIVLTQSPATLSLSPGERATLSCRASQSVTNYLAWHQQKPGQAPRLLIYDASNRATGIPARFSGSGSGTDFTLTISS
LEPEDFAVYYCQHRSNWPLTFGGGTKVEIK
VL CDR1 amino acid sequence: QSVTNY SEQ ID NO: 470
VL CDR2 amino acid sequence: DAS SEQ ID NO: 471
VL CDR3 amino acid sequence: QHRSNWPLT SEQ ID NO: 472
Antibody STIM008
VH domain nucleotide sequence: SEQ ID NO: 481
CAGATCACCTTGAAGGAGTCTGGTCCTACGCTGGTGAAACCCACACAGACCCTCACGCTGACCTGCACCTTCTCTGG
GTTCTCACTCAGCACTAGTGGAGTGGGTGTGGGCTGGATCCGTCAGCCCCCAGGAAAGGCCCTGGAGTGGCTTGCAG
TCATTTATTGGGATGATGATAAGCGCTACAGCCCATCTCTGAAGAGCAGGCTCACCATCACCAAGGACACCTCCAAA
AACCAGGTGGTCCTTACAATGACCAACATGGACCCTGTGGACACAGCCACATATTTCTGTACACACGGATATGGTTC
GGCGAGTTATTACCACTACGGTATGGACGTCTGGGGCCAAGGGACCACGGTCACCGTCTCCTCA
VH domain amino acid sequence: SEQ ID NO: 480
QITLKESGPTLVKPTQTLTLTCTFSGFSLSTSGVGVGWIRQPPGKALEWLAVIYWDDDKRYSPSLKSRLTITKDTSK
NQVVLTMTNMDPVDTATYFCTHGYGSASYYHYGMDVWGQGTTVTVSS
VH CDR1 amino acid sequence: GFSLSTSGVG SEQ ID NO: 477
VH CDR2 amino acid sequence: IYWDDDK SEQ ID NO: 478
VH CDR3 amino acid sequence: THGYGSASYYHYGMDV SEQ ID NO: 479
VL domain nucleotide sequence: SEQ ID NO: 488
GAAATTGTGTTGACACAGTCTCCAGCCACCCTGTCTTTGTCTCCAGGGGAAAGAGCCACCCTCTCCTGCAGGGCCAG
TCAGAGTGTTACCAACTACTTAGCCTGGCACCAACAGAAACCTGGCCAGGCTCCCAGGCTCCTCATCTATGATGCAT
CCAACAGGGCCACTGGCATCCCAGCCAGGTTCAGTGGCAGTGGGTCTGGGACAGACTTCACTCTCACCATCAGCAGC
CTAGAGCCTGAAGATTTTGCAGTTTATTACTGTCAGCAGCGTAGCAACTGGCCTCTCACTTTCGGCGGAGGGACCAA
GGTGGAGATCAAA
VL domain amino acid sequence: SEQ ID NO: 489
EIVLTQSPATLSLSPGERATLSCRASQSVTNYLAWHQQKPGQAPRLLIYDASNRATGIPARFSGSGSGTDFTLTISS
LEPEDFAVYYCQQRSNWPLTFGGGTKVEIK
VL CDR1 amino acid sequence: QSVTNY SEQ ID NO: 484
VL CDR2 amino acid sequence: DAS SEQ ID NO: 485
VL CDR3 amino acid sequence: QQRSNWPLT SEQ ID NO: 486
Antibody STIM009
VH domain nucleotide sequence: SEQ ID NO: 495
CAGGTGCAGCTGGTGGAGTCTGGGGGAGGCTTGGTCAAGCCTGGAGGGTCCCTGAGACTCTCCTGTGCAGCCTCTGG
ATTCACCTTCAGTGACTACTACATGAGCTGGATCCGCCAGGCTCCAGGGAAGGGGCTGGAGTGGGTTTCATACATTA
GTAGTAGTGGTAGTACCATATACTACGCAGACTCTGTGAAGGGCCGATTCACCATCTCCAGGGACAACGCCAAGAAC
TCACTGTATCTGCAAATTAACAGCCTGAGAGCCGAGGACACGGCCGTGTATTACTGTGCGAGAGATTTTTACGATAT
TTTGACTGATAGTCCGTACTTCTACTACGGTGTGGACGTCTGGGGCCAAGGGACCACGGTCACCGTCTCCTCA
VH domain amino acid sequence: SEQ ID NO: 494
QVQLVESGGGLVKPGGSLRLSCAASGFTFSDYYMSWIRQAPGKGLEWVSYISSSGSTIYYADSVKGRFTISRDNAKN
SLYLQINSLRAEDTAVYYCARDFYDILTDSPYFYYGVDVWGQGTTVTVSS
VH CDR1 amino acid sequence: GFTFSDYY SEQ ID NO: 491
VH CDR2 amino acid sequence: ISSSGSTI SEQ ID NO: 492
VH CDR3 amino acid sequence: ARDFYDILTDSPYFYYGVDV SEQ ID NO: 493
VL domain nucleotide sequence: SEQ ID NO: 502
GATATTGTGATGACTCAGTCTCCACTCTCCCTGCCCGTCACCCCTGGAGAGCCGGCCTCCATCTCCTGCAGGTCTAG
TCAGAGCCTCCTGCATAGTAATGGATACAACTATTTGGATTGGTACCTGCAGAAGCCAGGGCAGTCTCCACAGCTCC
TGATCTATTTGGGTTCTAATCGGGCCTCCGGGGTCCCTGACAGGTTCAGTGGCAGTGGATCAGGCACAGATTTTACA
CTGAAAATCAGCAGAGTGGAGGCTGAGGATGTTGGGGTTTATTACTGCATGCAAGCTCTACAAACTCCTCGGACGTT
CGGCCAAGGGACCAAGGTGGAAATCAAA
VL domain amino acid sequence: SEQ ID NO: 501
DIVMTQSPLSLPVTPGEPASISCRSSQSLLHSNGYNYLDWYLQKPGQSPQLLIYLGSNRASGVPDRFSGSGSGTDFT
LKISRVEAEDVGVYYCMQALQTPRTFGQGTKVEIK
VL CDR1 amino acid sequence: QSLLHSNGYNY SEQ ID NO: 498
VL CDR2 amino acid sequence: LGS SEQ ID NO: 499
VL CDR3 amino acid sequence: MQALQTPRT SEQ ID NO: 500

TABLE S1
SEQ ID NOS: 1-342

| SEQ | | | | |
|---|---|---|---|---|
| ID | | | | |
| NO: | Name | Description | | Sequence |
| 1 | Human | NCBI number: | | MRIFAVFIFMTYWHLLNAFTVTVPKDLYVVEYGSNMTIECKFPVEKQL |
| | PD-L1 | NP_054862.1 | | DLAALIVYWEMEDKNIIQFVHGEEDLKVQHSSYRQRARLLKDQLSLGN |
| | | (ECD highlighted in | | AALQITDVKLQDAGVYRCMISYGGADYKRITVKVNAPYNKINQRILVV |
| | | BOLD, cytoplasmic | | DPVTSEHELTCQAEGYPKAEVIWTSSDHQVLSGKTTTTNSKREEKLEN |
| | | domain underlined) | | VTSTLRINTTTNEIFYCTFRRLDPEENHTAELVIPELPLAHPPNERTH |
| | | | | LVILGAILLCLGVALTFIFRLRKGRMMDVKKCGIQDTNSKKQSDTHLE |
| | | | | ET |
| 2 | Cyno PD- | NCBI number: | | MGWSCIILFLVATATGVHSMFTVTVPKDLYVVEYGSNMTIECKFPVEK |
| | L1 | XP_014973154.1 | | QLDLTSLIVYWEMEDKNIIQFVHGEEDLKVQHSNYRQRAQLLKDQLSL |
| | | (ECD highlighted in | | GNAALRITDVKLQDAGVYRCMISYGGADYKRITVKVNAPYNKINQRIL |
| | | BOLD) | | VVDPVTSEHELTCQAEGYPKAEVIWTSSDHQVLSGKTTTTNSKREEKL |
| | | | | LNVTSTLRINTTANEIFYCIFRRLDPEENHTAELVIPELPLALPPNER |
| | | | | T |
| 3 | Human | Human PD-L1 ECD | | MRIFAVFIFMTYWHLLNAFTVTVPKDLYVVEYGSNMTIECKFPVEKQL |
| | PD-L1 | with C-terminal His | | DLAALIVYWEMEDKNIIQFVHGEEDLKVQHSSYRQRARLLKDQLSLGN |
| | His | tag | | AALQITDVKLQDAGVYRCMISYGGADYKRITVKVNAPYNKINQRILVV |
| | | | | DPVTSEHELTCQAEGYPKAEVIWTSSDHQVLSGKTTTTNSKREEKLEN |
| | | | | VTSTLRINTTTNEIFYCTFRRLDPEENHTAELVIPELPLAHPPNERTH |
| | | | | HHHHH |
| 4 | Human | Human PD-L1 ECD | | MRIFAVFIFMTYWHLLNAFTVTVPKDLYVVEYGSNMTIECKFPVEKQL |
| | PD-L1 Fc | with C-term Fc | | DLAALIVYWEMEDKNIIQFVHGEEDLKVQHSSYRQRARLLKDQLSLGN |
| | | fusion (in bold) | | AALQITDVKLQDAGVYRCMISYGGADYKRITVKVNAPYNKINQRILVV |
| | | | | DPVTSEHELTCQAEGYPKAEVIWTSSDHQVLSGKTTTTNSKREEKLEN |
| | | | | VTSTLRINTTTNEIFYCTFRRLDPEENHTAELVIPELPLAHPPNERTI |
| | | | | EGREPKSCDKTHTCPPCPAPELLGGPSVFLFPPKPKDTLMISRTPEVT |
| | | | | CVVVDVSHEDPEVKFNWYVDGVEVHNAKTKPREEQYNSTYRVVSVLTV |
| | | | | LHQDWLNGKEYKCKVSNKALPAPIEKTISKAKGQPREPQVYTLPPSRD |
| | | | | ELTKNQVSLTCLVKGFYPSDIAVEWESNGQPENNYKTTPPVLDSDGSF |
| | | | | FLYSKLTVDKSRWQQGNVFSCSVMHEALHNHYTQKSLSLSPGK |
| 5 | Cyno PD- | Cynomolgus PD-L1 | | MGWSCIILFLVATATGVHSMFTVTVPKDLYVVEYGSNMTIECKFPVEK |
| | L1 FLAG | ECD with N-term | | QLDLTSLIVYWEMEDKNIIQFVHGEEDLKVQHSNYRQRAQLLKDQLSL |
| | | FLAG tag | | GNAALRITDVKLQDAGVYRCMISYGGADYKRITVKVNAPYNKINQRIL |
| | | | | VVDPVTSEHELTCQAEGYPKAEVIWTSSDHQVLSGKTTTTNSKREEKL |
| | | | | LNVTSTLRINTTANEIFYCIFRRLDPEENHTAELVIPELPLALPPNER |
| | | | | TDYKDDDDK |
| 6 | Human | Human PD-1 full | | MGWSCIILFLVATATGVHSLDSPDRPWNPPTFSPALLVVTEGDNATFT |
| | PD-1 Fc | length sequence | | CSFSNTSESFVLNWYRMSPSNQTDKLAAFPEDRSQPGQDCRFRVTQLP |
| | | derived from cDNA | | NGRDFHMSVVRARRNDSGTYLCGAISLAPKAQIKESLRAELRVTERRA |
| | | as human Fc fusion | | EVPTAHPSPSPRPAGQKLENLYFQGIEGRMDEPKSCDKTHTCPPCPAP |
| | | | | ELLGGPSVFLFPPKPKDTLMISRTPEVTCVVVDVSHEDPEVKFNWYVD |
| | | | | GVEVHNAKTKPREEQYNSTYRVVSVLTVLHQDWLNGKEYKCKVSNKAL |
| | | | | PAPIEKTISKAKGQPREPQVYTLPPSRDELTKNQVSLTCLVKGFYPSD |
| | | | | IAVEWESNGQPENNYKTTPPVLDSDGSFFLYSKLTVDKSRWQQGNVFS |
| | | | | CSVMHEALHNHYTQKSLSLSP |
| 7 | 84G09- | Amino acid sequence | | GFTFDDYA |
| | CDRH1 | of CDRH1 of 84G09 | | |
| | (IMGT) | using IMGT | | |
| 8 | 84G09- | Amino acid sequence | | ISWKSNII |
| | CDRH2 | of CDRH2 of 84G09 | | |
| | (IMGT) | using IMGT | | |
| 9 | 84G09- | Amino acid sequence | | ARDITGSGSYGWFDP |
| | CDRH3 | of CDRH3 of 84G09 | | |
| | (IMGT) | using IMGT | | |
| 10 | 84G09- | Amino acid sequence | | DYAMH |
| | CDRH1 | of CDRH1 of 84G09 | | |
| | (Kabat) | using Kabat | | |
| 11 | 84G09- | Amino acid sequence | | GISWKSNIIGYADSVKG |
| | CDRH2 | of CDRH2 of 84G09 | | |
| | (Kabat) | using Kabat | | |
| 12 | 84G09- | Amino acid sequence | | DITGSGSYGWFDP |
| | CDRH3 | of CDRH3 of 84G09 | | |
| | (Kabat) | using Kabat | | |
| 13 | 84G09- | Amino acid sequence | | EVQLVESGGGLVQPGRSLRLSCAASGFTFDDYAMHWVRQTPGKGLEWV |
| | Heavy | of V_{H} of | | SGISWKSNIIGYADSVKGRFTISRDNAKNSLYLQMNSLRAEDTALYYC |
| | chain | 84G09 (mutations | | ARDITGSGSYGWEDPWGQGTLVTVSS |
| | variable | from germline are | | |
| | region | shown in bold | | |
| | | letters) | | |
| 14 | 84G09- | Nucleic acid | | CAaGAAAAAGCTTGCCGCCACCATGGAGTTTGGGCTGAGCTGGATTTT |
| | Heavy | sequence of V_{H} of | | CCTTTTGGCTATTTTAAAAGGTGTCCAGTGTGAAGTACAATTGGTGGA |
| | chain | 84G09 | | GTCCGGGGGAGGCTTGGTACAGCCTGGCAGGTCCCTGAGACTCTCCTG |
| | variable | | | TGCAGCCTCTGGATTCACCTTTGATGATTATGCCATGCACTGGGTCCG |
| | region | | | ACAAACTCCAGGGAAGGGCCTGGAGTGGGTCTCAGGTATAAGTTGGAA |
| | | | | GAGTAATATCATAGGCTATGCGGACTCTGTGAAGGGCCGATTCACCAT |
| | | | | CTCCAGAGACAACGCCAAGAACTCCCTGTATCTGCAAATGAACAGTCT |
| | | | | GAGAGCTGAGGACACGGCCTTGTATTATTGTGCAAGAGATATAACGGG |
| | | | | TTCGGGGAGTTATGGCTGGTTCGACCCCTGGGGCCAGGGAACCCTGGT |
| | | | | CACCGTCTCCTCAGCCAAAACGACACCCCCATCTGTCTATCCACTGGC |
| | | | | CCCTGAATCTGCTAAAACTCAGCCTCCG |
| 15 | 84G09- | Amino acid sequence | | EVQLVESGGGLVQPGRSLRLSCAASGFTFDDYAMHWVRQTPGKGLEWV |
| | full | of 84G09 heavy | | SGISWKSNIIGYADSVKGRFTISRDNAKNSLYLQMNSLRAEDTALYYC |
| | heavy | chain (mutations | | ARDITGSGSYGWEDPWGQGTLVTVSSASTKGPSVFPLAPCSRSTSEST |
| | chain | from germline are | | AALGCLVKDYFPEPVTVSWNSGALTSGVHTFPAVLQSSGLYSLSSVVT |
| | sequence | shown in bold | | VPSSSLGTKTYTCNVDHKPSNTKVDKRVESKYGPPCPPCPAPEFEGGP |
| | | letters) | | SVFLFPPKPKDTLMISRTPEVTCVVVDVSQEDPEVQFNWYVDGVEVHN |
| | | | | AKTKPREEQFNSTYRVVSVLTVLHQDWLNGKEYKCKVSNKGLPSSIEK |
| | | | | TISKAKGQPREPQVYTLPPSQEEMTKNQVSLTCLVKGFYPSDIAVEWE |
| | | | | SNGQPENNYKTTPPVLDSDGSFFLYSRLTVDKSRWQEGNVFSCSVMHE |
| | | | | ALHNHYTQKSLSLSLGK |
| 16 | 84G09- | Nucleic acid | | GAAGTGCAGCTGGTGGAATCTGGCGGCGGACTGGTGCAGCCTGGCAGA |
| | full | sequence of 84G09 | | TCCCTGAGACTGTCTTGTGCCGCCTCCGGCTTCACCTTCGACGACTAC |
| | heavy | heavy chain | | GCTATGCACTGGGTGCGACAGACCCCTGGCAAGGGCCTGGAATGGGTG |
| | chain | | | TCCGGCATCTCCTGGAAGTCCAACATCATCGGCTACGCCGACTCCGTG |
| | sequence | | | AAGGGCCGGTTCACCATCTCCCGGGACAACGCCAAGAACTCCCTGTAC |
| | | | | CTGCAGATGAACAGCCTGCGGGCCGAGGACACCGCCCTGTACTACTGC |
| | | | | GCCAGAGACATCACCGGCTCCGGCTCCTACGGATGGTTCGATCCTTGG |
| | | | | GGCCAGGGCACCCTCGTGACCGTGTCCTCTGCCAGCACCAAGGGCCCC |
| | | | | TCTGTGTTCCCTCTGGCCCCTTCCAGCAAGTCCACCTCTGGCGGAACA |
| | | | | GCCGCTCTGGGCTGCCTCGTGAAGGACTACTTCCCCGAGCCTGTGACC |
| | | | | GTGTCCTGGAACTCTGGCGCTCTGACCAGCGGAGTGCACACCTTCCCT |
| | | | | GCTGTGCTGCAGTCCTCCGGCCTGTACTCCCTGTCCTCCGTCGTGACC |
| | | | | GTGCCTTCCAGCTCTCTGGGCACCCAGACCTACATCTGCAACGTGAAC |
| | | | | CACAAGCCCTCCAACACCAAGGTGGACAAGAAGGTGGAACCCAAGTCC |
| | | | | TGCGACAAGACCCACACCTGTCCCCCTTGTCCTGCCCCTGAACTGCTG |
| | | | | GGCGGACCTTCCGTGTTCCTGTTCCCCCCAAAGCCCAAGGACACCCTG |
| | | | | ATGATCTCCCGGACCCCCGAAGTGACCTGCGTGGTGGTGGATGTGTCC |
| | | | | CACGAGGACCCTGAAGTGAAGTTCAATTGGTACGTGGACGGCGTGGAA |
| | | | | GTGCACAACGCCAAGACCAAGCCTAGAGAGGAACAGTACAACTCCACC |
| | | | | TACCGGGTGGTGTCCGTGCTGACCGTGCTGCACCAGGATTGGCTGAAC |
| | | | | GGCAAAGAGTACAAGTGCAAGGTGTCCAACAAGGCCCTGCCTGCCCCC |
| | | | | ATCGAAAAGACCATCTCCAAGGCCAAGGGCCAGCCCCGGGAACCCCAG |
| | | | | GTGTACACACTGCCCCCTAGCAGGGACGAGCTGACCAAGAACCAGGTG |
| | | | | TCCCTGACCTGTCTCGTGAAAGGCTTCTACCCCTCCGATATCGCCGTG |
| | | | | GAATGGGAGTCCAACGGCCAGCCTGAGAACAACTACAAGACCACCCCC |
| | | | | CCTGTGCTGGACTCCGACGGCTCATTCTTCCTGTACAGCAAGCTGACA |
| | | | | GTGGACAAGTCCCGGTGGCAGCAGGGCAACGTGTTCTCCTGCTCCGTG |
| | | | | ATGCACGAGGCCCTGCACAACCACTACACCCAGAAGTCCCTGTCCCTG |
| | | | | AGCCCCGGCAAG |
| 17 | 84G09- | Amino acid sequence | | QSISSY |
| | CDRL1 | of CDRL1 of 84G09 | | |
| | (IMGT) | using IMGT | | |
| 18 | 84G09- | Amino acid sequence | | VAS |
| | CDRL2 | of CDRL2 of 84G09 | | |
| | (IMGT) | using IMGT | | |
| 19 | 84G09- | Amino acid sequence | | QQSYSNPIT |
| | CDRL3 | of CDRL3 of 84G09 | | |
| | (IMGT) | using IMGT | | |
| 20 | 84G09- | Amino acid sequence | | RASQSISSYLN |
| | CDRL1 | of CDRL1 of 84G09 | | |
| | (Kabat) | using Kabat | | |
| 21 | 84G09- | Amino acid sequence | | VASSLQS |
| | CDRL2 | of CDRL2 of 84G09 | | |
| | (Kabat) | using Kabat | | |
| 22 | 84G09- | Amino acid sequence | | QQSYSNPIT |
| | CDRL3 | of CDRL3 of 84G09 | | |
| | (Kabat) | using Kabat | | |
| 23 | 84G09- | Amino acid sequence | | DIQMTQSPSSLSASVGDRVTITCRASQSISSYLNWYQQKPGKAPKPLI |
| | Light | of V_{L} of 84G09 | | YVASSLQSGVPSSFSGSGSGTDFTLTISSLQPEDFATYYCQQSYSNPI |
| | chain | | | TFGQGTRLEIK |
| | variable | | | |
| | region | | | |
| 24 | 84G09- | Nucleic acid | | GACATCCAGATGACCCAGTCTCCATCCTCCCTGTCTGCATCTGTAGGA |
| | Light | sequence of V_{L} of | | GACAGAGTCACCATCACTTGCCGGGCAAGTCAGAGCATTAGCAGCTAT |
| | chain | 84G09 | | TTAAATTGGTATCAGCAGAAACCAGGGAAAGCCCCTAAGCCCCTGATC |
| | variable | | | TATGTTGCATCCAGTTTGCAAAGTGGGGTCCCATCAAGTTTCAGTGGC |
| | region | | | AGTGGATCTGGGACAGATTTCACTCTCACCATCAGCAGTCTGCAACCT |
| | | | | GAAGATTTTGCAACTTACTACTGTCAACAGAGTTACAGTAATCCGATC |
| | | | | ACCTTCGGCCAAGGGACACGACTGGAGATCAAA |
| 25 | 84G09- | Amino acid sequence | | DIQMTQSPSSLSASVGDRVTITCRASQSISSYLNWYQQKPGKAPKPLI |
| | full | of 84G09 light | | YVASSLQSGVPSSFSGSGSGTDFTLTISSLQPEDFATYYCQQSYSNPI |
| | light | chain | | TFGQGTRLEIKRTVAAPSVFIFPPSDEQLKSGTASVVCLLNNFYPREA |
| | chain | | | KVQWKVDNALQSGNSQESVTEQDSKDSTYSLSSTLTLSKADYEKHKVY |
| | sequence | | | ACEVTHQGLSSPVTKSFNRGEC |
| 26 | 84G09- | Nucleic acid | | GACATCCAGATGACCCAGTCTCCATCCTCCCTGTCTGCATCTGTAGGA |
| | full | sequence of 84G09 | | GACAGAGTCACCATCACTTGCCGGGCAAGTCAGAGCATTAGCAGCTAT |
| | light | light chain | | TTAAATTGGTATCAGCAGAAACCAGGGAAAGCCCCTAAGCCCCTGATC |
| | chain | | | TATGTTGCATCCAGTTTGCAAAGTGGGGTCCCATCAAGTTTCAGTGGC |
| | sequence | | | AGTGGATCTGGGACAGATTTCACTCTCACCATCAGCAGTCTGCAACCT |
| | | | | GAAGATTTTGCAACTTACTACTGTCAACAGAGTTACAGTAATCCGATC |
| | | | | ACCTTCGGCCAAGGGACACGACTGGAGATCAAACGTACGGTGGCCGCT |
| | | | | CCCTCCGTGTTCATCTTCCCACCTTCCGACGAGCAGCTGAAGTCCGGC |
| | | | | ACCGCTTCTGTCGTGTGCCTGCTGAACAACTTCTACCCCCGCGAGGCC |
| | | | | AAGGTGCAGTGGAAGGTGGACAACGCCCTGCAGTCCGGCAACTCCCAG |
| | | | | GAATCCGTGACCGAGCAGGACTCCAAGGACAGCACCTACTCCCTGTCC |
| | | | | TCCACCCTGACCCTGTCCAAGGCCGACTACGAGAAGCACAAGGTGTAC |
| | | | | GCCTGCGAAGTGACCCACCAGGGCCTGTCTAGCCCCGTGACCAAGTCT |
| | | | | TTCAACCGGGGCGAGTGT |
| 27 | 1D05- | Amino acid sequence | | GFTFDDYA |
| | CDRH1 | of CDRH1 of 1D05 | | |
| | (IMGT) | using IMGT | | |
| 28 | 1D05- | Amino acid sequence | | ISWIRTGI |
| | CDRH2 | of CDRH2 of 1D05 | | |
| | (IMGT) | using IMGT | | |
| 29 | 1D05- | Amino acid sequence | | AKDMKGSGTYGGWFDT |
| | CDRH3 | of CDRH3 of 1D05 | | |
| | (IMGT) | using IMGT | | |
| 30 | 1D05- | Amino acid sequence | | DYAMH |
| | CDRH1 | of CDRH1 of 1D05 | | |
| | (Kabat) | using Kabat | | |
| 31 | 1D05- | Amino acid sequence | | GISWIRTGIGYADSVKG |
| | CDRH2 | of CDRH2 of 1D05 | | |
| | (Kabat) | using Kabat | | |
| 32 | 1D05- | Amino acid sequence | | DMKGSGTYGGWFDT |
| | CDRH3 | of CDRH3 of 1D05 | | |
| | (Kabat) | using Kabat | | |
| 33 | 1D05- | Amino acid sequence | | EVQLVESGGGLVQPGRSLRLSCAASGFTFDDYAMHWVRQVPGKGLEWV |
| | Heavy | of V_{H} of 1D05 | | SGISWIRTGIGYADSVKGRFTIFRDNAKNSLYLQMNSLRAEDTALYYC |
| | chain | (mutations from | | AKDMKGSGTYGGWFDTWGQGTLVTVSS |
| | variable | germline are shown | | |
| | region | in bold letters) | | |
| 34 | 1D05- | Nucleic acid | | AAGCTTGCCGCCACCATGGAGTTTGGGCTGAGCTGGATTTTCCTTTTG |
| | Heavy | sequence of V_{H} of | | GCTATTTTAAAAGGTGTCCAGTGTGAAGTGCAGCTGGTGGAGTCTGGG |
| | chain | 1D05 | | GGAGGCTTGGTGCAGCCTGGCAGGTCCCTGAGACTCTCCTGTGCAGCC |
| | variable | | | TCTGGATTCACCTTTGATGATTATGCCATGCACTGGGTCCGGCAAGTT |
| | region | | | CCAGGGAAGGGCCTGGAATGGGTCTCAGGCATTAGTTGGATTCGTACT |
| | | | | GGCATAGGCTATGCGGACTCTGTGAAGGGCCGATTCACCATTTTCAGA |
| | | | | GACAACGCCAAGAATTCCCTGTATCTGCAAATGAACAGTCTGAGAGCT |
| | | | | GAGGACACGGCCTTGTATTACTGTGCAAAAGATATGAAGGGTTCGGGG |
| | | | | ACTTATGGGGGGTGGTTCGACACCTGGGGCCAGGGAACCCTGGTCACC |
| | | | | GTCTCCTCAGCCAAAACAACAGCCCCATCGGTCTATCCACTGGCCCCT |
| | | | | GC |
| 35 | 1D05- | Amino acid sequence | | EVQLVESGGGLVQPGRSLRLSCAASGFTFDDYAMHWVRQVPGKGLEWV |
| | full | of 1D05 heavy chain | | SGISWIRTGIGYADSVKGRFTIFRDNAKNSLYLQMNSLRAEDTALYYC |
| | heavy | | | AKDMKGSGTYGGWFDTWGQGTLVTVSSASTKGPSVFPLAPCSRSTSES |
| | chain | | | TAALGCLVKDYFPEPVTVSWNSGALTSGVHTFPAVLQSSGLYSLSSVV |
| | sequence | | | TVPSSSLGTKTYTCNVDHKPSNTKVDKRVESKYGPPCPPCPAPEFEGG |
| | | | | PSVFLFPPKPKDTLMISRTPEVTCVVVDVSQEDPEVQFNWYVDGVEVH |
| | | | | NAKTKPREEQFNSTYRVVSVLTVLHQDWLNGKEYKCKVSNKGLPSSIE |
| | | | | KTISKAKGQPREPQVYTLPPSQEEMTKNQVSLTCLVKGFYPSDIAVEW |
| | | | | ESNGQPENNYKTTPPVLDSDGSFFLYSRLTVDKSRWQEGNVFSCSVMH |
| | | | | EALHNHYTQKSLSLSLGK |
| 36 | 1D05- | Nucleic acid | | GAAGTGCAGCTGGTGGAATCTGGCGGCGGACTGGTGCAGCCTGGCAGA |
| | full | sequence of 1D05 | | TCCCTGAGACTGTCTTGTGCCGCCTCCGGCTTCACCTTCGACGACTAC |
| | heavy | heavy chain | | GCTATGCACTGGGTGCGACAGGTGCCAGGCAAGGGCCTGGAATGGGTG |
| | chain | | | TCCGGCATCTCTTGGATCCGGACCGGCATCGGCTACGCCGACTCTGTG |
| | sequence | | | AAGGGCCGGTTCACCATCTTCCGGGACAACGCCAAGAACTCCCTGTAC |
| | | | | CTGCAGATGAACAGCCTGCGGGCCGAGGACACCGCCCTGTACTACTGC |
| | | | | GCCAAGGACATGAAGGGCTCCGGCACCTACGGCGGATGGTTCGATACT |
| | | | | TGGGGCCAGGGCACCCTCGTGACCGTGTCCTCTGCCAGCACCAAGGGC |
| | | | | CCCTCTGTGTTCCCTCTGGCCCCTTCCAGCAAGTCCACCTCTGGCGGA |
| | | | | ACAGCCGCTCTGGGCTGCCTCGTGAAGGACTACTTCCCCGAGCCTGTG |
| | | | | ACCGTGTCCTGGAACTCTGGCGCTCTGACCAGCGGAGTGCACACCTTC |
| | | | | CCTGCTGTGCTGCAGTCCTCCGGCCTGTACTCCCTGTCCTCCGTCGTG |
| | | | | ACCGTGCCTTCCAGCTCTCTGGGCACCCAGACCTACATCTGCAACGTG |
| | | | | AACCACAAGCCCTCCAACACCAAGGTGGACAAGAAGGTGGAACCCAAG |
| | | | | TCCTGCGACAAGACCCACACCTGTCCCCCTTGTCCTGCCCCTGAACTG |
| | | | | CTGGGCGGACCTTCCGTGTTCCTGTTCCCCCCAAAGCCCAAGGACACC |
| | | | | CTGATGATCTCCCGGACCCCCGAAGTGACCTGCGTGGTGGTGGATGTG |
| | | | | TCCCACGAGGACCCTGAAGTGAAGTTCAATTGGTACGTGGACGGCGTG |
| | | | | GAAGTGCACAACGCCAAGACCAAGCCTAGAGAGGAACAGTACAACTCC |
| | | | | ACCTACCGGGTGGTGTCCGTGCTGACCGTGCTGCACCAGGATTGGCTG |
| | | | | AACGGCAAAGAGTACAAGTGCAAGGTGTCCAACAAGGCCCTGCCTGCC |
| | | | | CCCATCGAAAAGACCATCTCCAAGGCCAAGGGCCAGCCCCGGGAACCC |
| | | | | CAGGTGTACACACTGCCCCCTAGCAGGGACGAGCTGACCAAGAACCAG |
| | | | | GTGTCCCTGACCTGTCTCGTGAAAGGCTTCTACCCCTCCGATATCGCC |
| | | | | GTGGAATGGGAGTCCAACGGCCAGCCTGAGAACAACTACAAGACCACC |
| | | | | CCCCCTGTGCTGGACTCCGACGGCTCATTCTTCCTGTACAGCAAGCTG |
| | | | | ACAGTGGACAAGTCCCGGTGGCAGCAGGGCAACGTGTTCTCCTGCTCC |
| | | | | GTGATGCACGAGGCCCTGCACAACCACTACACCCAGAAGTCCCTGTCC |
| | | | | CTGAGCCCCGGCAAG |
| 37 | 1D05- | Amino acid sequence | | QSISSY |
| | CDRL1 | of CDRL1 of 1D05 | | |
| | (IMGT) | using IMGT | | |
| 38 | 1D05- | Amino acid sequence | | VAS |
| | CDRL2 | of CDRL2 of 1D05 | | |
| | (IMGT) | using IMGT | | |
| 39 | 1D05- | Amino acid sequence | | QQSYSTPIT |
| | CDRL3 | of CDRL3 of 1D05 | | |
| | (IMGT) | using IMGT | | |
| 40 | 1D05- | Amino acid sequence | | RASQSISSYLN |
| | CDRL1 | of CDRL1 of 1D05 | | |
| | (Kabat) | using Kabat | | |
| 41 | 1D05- | Amino acid sequence | | VASSLQS |
| | CDRL2 | of CDRL2 of 1D05 | | |
| | (Kabat) | using Kabat | | |
| 42 | 1D05- | Amino acid sequence | | QQSYSTPIT |
| | CDRL3 | of CDRL3 of 1D05 | | |
| | (Kabat) | using Kabat | | |
| 43 | 1D05- | Amino acid sequence | | DIQMTQSPSSLSASVGDRVTITCRASQSISSYLNWYQQKPGKAPKLLI |
| | Light | of V_{L} of | | YVASSLQSGVPSRFSGSGSGTDFTLTISSLQPEDFATYYCQQSYSTPI |
| | chain | 1D05 (mutations from | | TFGQGTRLEIK |
| | variable | germline are shown | | |
| | region | in bold letters) | | |
| 44 | 1D05- | Nucleic acid | | AAAGCTTGCCGCCACCATGAGGCTCCCTGCTCAGCTTCTGGGGCTCCT |
| | Light | sequence of V_{L} of | | GCTACTCTGGCTCCGAGGTGCCAGATGTGACATCCAGATGACCCAGTC |
| | chain | 1D05 | | TCCATCCTCCCTGTCTGCATCTGTAGGAGACAGAGTCACCATCACTTG |
| | variable | | | CCGGGCAAGTCAGAGCATTAGCAGCTATTTAAATTGGTATCAGCAGAA |
| | region | | | ACCAGGGAAAGCCCCTAAACTCCTGATCTATGTTGCATCCAGTTTGCA |
| | | | | AAGTGGGGTCCCATCAAGGTTCAGTGGCAGTGGATCTGGGACAGATTT |
| | | | | CACTCTCACTATCAGCAGTCTGCAACCTGAAGATTTTGCAACTTACTA |
| | | | | CTGTCAACAGAGTTACAGTACCCCGATCACCTTCGGCCAAGGGACACG |
| | | | | TCTGGAGATCAAACGTACGGATGCTGCACCAACT |
| 45 | 1D05- | Amino acid sequence | | DIQMTQSPSSLSASVGDRVTITCRASQSISSYLNWYQQKPGKAPKLLI |
| | full | of 1D05 light chain | | YVASSLQSGVPSRFSGSGSGTDFTLTISSLQPEDFATYYCQQSYSTPI |
| | light | | | TFGQGTRLEIKRTVAAPSVFIFPPSDEQLKSGTASVVCLLNNFYPREA |
| | chain | | | KVQWKVDNALQSGNSQESVTEQDSKDSTYSLSSTLTLSKADYEKHKVY |
| | sequence | | | ACEVTHQGLSSPVTKSFNRGEC |
| 46 | 1D05- | Nucleic acid | | GACATCCAGATGACCCAGTCCCCCTCCAGCCTGTCTGCTTCCGTGGGC |
| | full | sequence of 1D05 | | GACAGAGTGACCATCACCTGTCGGGCCTCCCAGTCCATCTCCTCCTAC |
| | light | light chain | | CTGAACTGGTATCAGCAGAAGCCCGGCAAGGCCCCCAAGCTGCTGATC |
| | chain | | | TACGTGGCCAGCTCTCTGCAGTCCGGCGTGCCCTCTAGATTCTCCGGC |
| | sequence | | | TCTGGCTCTGGCACCGACTTTACCCTGACCATCAGCTCCCTGCAGCCC |
| | | | | GAGGACTTCGCCACCTACTACTGCCAGCAGTCCTACTCCACCCCTATC |
| | | | | ACCTTCGGCCAGGGCACCCGGCTGGAAATCAAACGTACGGTGGCCGCT |
| | | | | CCCTCCGTGTTCATCTTCCCACCTTCCGACGAGCAGCTGAAGTCCGGC |
| | | | | ACCGCTTCTGTCGTGTGCCTGCTGAACAACTTCTACCCCCGCGAGGCC |
| | | | | AAGGTGCAGTGGAAGGTGGACAACGCCCTGCAGTCCGGCAACTCCCAG |
| | | | | GAATCCGTGACCGAGCAGGACTCCAAGGACAGCACCTACTCCCTGTCC |
| | | | | TCCACCCTGACCCTGTCCAAGGCCGACTACGAGAAGCACAAGGTGTAC |
| | | | | GCCTGCGAAGTGACCCACCAGGGCCTGTCTAGCCCCGTGACCAAGTCT |
| | | | | TTCAACCGGGGCGAGTGT |
| 47 | Mutated | Amino acid sequence | | EVQLVESGGGLVQPGRSLRLSCAASGFTFDDYAMHWVRQAPGKGLEWV |
| | 1D05- | of 1D05 heavy chain | | SGISWIRTGIGYADSVKGRFTIFRDNAKNSLYLQMNSLRAEDTALYYC |
| | HC | with V to A back- | | AKDMKGSGTYGGWFDTWGQGTLVTVSSASTKGPSVFPLAPCSRSTSES |
| | mutant 1 | mutation in | | TAALGCLVKDYFPEPVTVSWNSGALTSGVHTFPAVLQSSGLYSLSSVV |
| | | framework region to | | TVPSSSLGTKTYTCNVDHKPSNTKVDKRVESKYGPPCPPCPAPELAGA |
| | | germline | | PSVFLFPPKPKDTLMISRTPEVTCVVVDVSQEDPEVQFNWYVDGVEVH |
| | | highlighted with | | NAKTKPREEQFNSTYRVVSVLTVLHQDWLNGKEYKCKVSNKGLPSSIE |
| | | IgG1 disabled | | KTISKAKGQPREPQVYTLPPSQEEMTKNQVSLTCLVKGFYPSDIAVEW |
| | | (LAGA) constant | | ESNGQPENNYKTTPPVLDSDGSFFLYSRLTVDKSRWQEGNVFSCSVMH |
| | | region | | EALHNHYTQKSLSLSLGK |
| 48 | Mutated | Amino acid sequence | | EVQLVESGGGLVQPGRSLRLSCAASGFTFDDYAMHWVRQVPGKGLEWV |
| | 1D05- | of 1D05 heavy chain | | SGISWIRTGIGYADSVKGRFTISRDNAKNSLYLQMNSLRAEDTALYYC |
| | HC | with F to S back- | | AKDMKGSGTYGGWFDTWGQGTLVTVSSASTKGPSVFPLAPCSRSTSES |
| | mutant 2 | mutation in | | TAALGCLVKDYFPEPVTVSWNSGALTSGVHTFPAVLQSSGLYSLSSVV |
| | | framework region to | | TVPSSSLGTKTYTCNVDHKPSNTKVDKRVESKYGPPCPPCPAPELAGA |
| | | germline | | PSVFLFPPKPKDTLMISRTPEVTCVVVDVSQEDPEVQFNWYVDGVEVH |
| | | highlighted with | | NAKTKPREEQFNSTYRVVSVLTVLHQDWLNGKEYKCKVSNKGLPSSIE |
| | | IgG1 disabled | | KTISKAKGQPREPQVYTLPPSQEEMTKNQVSLTCLVKGFYPSDIAVEW |
| | | (LAGA) constant | | ESNGQPENNYKTTPPVLDSDGSFFLYSRLTVDKSRWQEGNVFSCSVMH |
| | | region | | EALHNHYTQKSLSLSLGK |
| 49 | Mutated | Amino acid sequence | | EVQLVESGGGLVQPGRSLRLSCAASGFTFDDYAMHWVRQVPGKGLEWV |
| | 1D05- | of 1D05 heavy chain | | SGISWIRTGIGYADSVKGRFTIFRDNAKNSLYLQMNSLRAEDTALYYC |
| | HC | with ELLG to -PVA | | AKDMKGSGTYGGWFDTWGQGTLVTVSSASTKGPSVFPLAPCSRSTSES |
| | mutant 3 | back-mutation in | | TAALGCLVKDYFPEPVTVSWNSGALTSGVHTFPAVLQSSGLYSLSSVV |
| | | constant region to | | TVPSSSLGTKTYTCNVDHKPSNTKVDKRVESKYGPPCPPCPAP- |
| | | germline | | PVAGPSVFLFPPKPKDTLMISRTPEVTCVVVDVSQEDPEVQFNWYVDG |
| | | highlighted | | VEVHNAKTKPREEQFNSTYRVVSVLTVLHQDWLNGKEYKCKVSNKGLP |
| | | | | SSIEKTISKAKGQPREPQVYTLPPSQEEMTKNQVSLTCLVKGFYPSDI |
| | | | | AVEWESNGQPENNYKTTPPVLDSDGSFFLYSRLTVDKSRWQEGNVFSC |
| | | | | SVMHEALHNHYTQKSLSLSLGK |
| 50 | Mutated | Amino acid sequence | | DIQMTQSPSSLSASVGDRVTITCRASQSISSYLNWYQQKPGKAPKLLI |
| | 1D05- | of 1D05 appa light | | YAASSLQSGVPSRFSGSGSGTDFTLTISSLQPEDFATYYCQQSYSTPI |
| | LC | chain with V to A | | TFGQGTRLEIKRTVAAPSVFIFPPSDEQLKSGTASVVCLLNNFYPREA |
| | mutant 1 | back-mutation in | | KVQWKVDNALQSGNSQESVTEQDSKDSTYSLSSTLTLSKADYEKHKVY |
| | | CDRL2 to germline | | ACEVTHQGLSSPVTKSFNRGEC |
| | | highlighted | | |
| 51 | Mutated | Amino acid sequence | | DIQMTQSPSSLSASVGDRVTITCRASQSISSYLNWYQQKPGKAPKL FI |
| | 1D05- | of 1D05 kappa light | | YVASSLQSGVPSRFSGSGSGTDFTLTISSLQPEDFATYYCQQSYSTPI |
| | LC | chain with L to F | | TFGQGTRLEIKRTVAAPSVFIFPPSDEQLKSGTASVVCLLNNFYPREA |
| | mutant 2 | back-mutation in | | KVQWKVDNALQSGNSQESVTEQDSKDSTYSLSSTLTLSKADYEKHKVY |
| | | framework to | | ACEVTHQGLSSPVTKSFNRGEC |
| | | germline | | |
| | | highlighted | | |
| 52 | 411B08- | Amino acid sequence | | GFTFSSYW |
| | CDRH1 | of CDRH1 of 411B08 | | |
| | (IMGT) | using IMGT | | |
| 53 | 411B08- | Amino acid sequence | | IKEDGSEK |
| | CDRH2 | of CDRH2 of 411B08 | | |
| | (IMGT) | using IMGT | | |
| 54 | 411B08- | Amino acid sequence | | ARNRLYSDFLDN |
| | CDRH3 | of CDRH3 of 411B08 | | |
| | (IMGT) | using IMGT | | |
| 55 | 411B08- | Amino acid sequence | | SYWMS |
| | CDRH1 | of CDRH1 of 411B08 | | |
| | (Kabat) | using Kabat | | |
| 56 | 411B08- | Amino acid sequence | | NIKEDGSEKYYVDSVKG |
| | CDRH2 | of CDRH2 of 411B08 | | |
| | (Kabat) | using Kabat | | |
| 57 | 411B08- | Amino acid sequence | | NRLYSDFLDN |
| | CDRH3 | of CDRH3 of 411B08 | | |
| | (Kabat) | using Kabat | | |
| 58 | 411B08- | Amino acid sequence | | EVQLVESGGGLVQPGGSLRLSCAASGFTFSSYWMSWVRQAPGKGLEWV |
| | Heavy | of V_{H} of 411B08 | | ANIKEDGSEKYYVDSVKGRFTISRDNAKNSLYLQMNSLRAEDTSVYYC |
| | chain | | | ARNRLYSDFLDNWGQGTLVTVSS |
| | variable | | | |
| | region | | | |
| 59 | 411B08- | Nucleic acid | | GAGGTGCAGCTGGTGGAGTCTGGGGGAGGCTTGGTCCAGCCTGGGGGG |
| | Heavy | sequence of V_{H} of | | TCCCTGAGACTCTCCTGTGCAGCCTCTGGATTCACGTTTAGTAGCTAT |
| | chain | 411B08 | | TGGATGAGTTGGGTCCGCCAGGCTCCAGGGAAGGGGCTGGAGTGGGTG |
| | variable | | | GCCAACATCAAAGAAGATGGAAGTGAGAAATACTATGTCGACTCTGTG |
| | region | | | AAGGGCCGATTCACCATCTCCAGAGACAACGCCAAGAACTCACTGTAT |
| | | | | CTGCAAATGAACAGCCTGAGAGCCGAGGACACGTCTGTGTATTACTGT |
| | | | | GCGAGAAATCGACTCTACAGTGACTTCCTTGACAACTGGGGCCAGGGA |
| | | | | ACCCTGGTCACCGTCTCCTCAG |
| 60 | 411B08- | Amino acid sequence | | EVQLVESGGGLVQPGGSLRLSCAASGFTFSSYWMSWVRQAPGKGLEWV |
| | full | of 411B08 heavy | | ANIKEDGSEKYYVDSVKGRFTISRDNAKNSLYLQMNSLRAEDTSVYYC |
| | heavy | chain | | ARNRLYSDFLDNWGQGTLVTVSSASTKGPSVFPLAPSSKSTSGGTAAL |
| | chain | | | GCLVKDYFPEPVTVSWNSGALTSGVHTFPAVLQSSGLYSLSSVVTVPS |
| | sequence | | | SSLGTQTYICNVNHKPSNTKVDKKVEPKSCDKTHTCPPCPAPELLGGP |
| | | | | SVFLFPPKPKDTLMISRTPEVTCVVVDVSHEDPEVKFNWYVDGVEVHN |
| | | | | AKTKPREEQYNSTYRVVSVLTVLHQDWLNGKEYKCKVSNKALPAPIEK |
| | | | | TISKAKGQPREPQVYTLPPSRDELTKNQVSLTCLVKGFYPSDIAVEWE |
| | | | | SNGQPENNYKTTPPVLDSDGSFFLYSKLTVDKSRWQQGNVFSCSVMHE |
| | | | | ALHNHYTQKSLSLSPGK |
| 61 | 411B08- | Nucleic acid | | GAGGTGCAGCTGGTGGAGTCTGGGGGAGGCTTGGTCCAGCCTGGGGGG |
| | full | sequence of 411B08 | | TCCCTGAGACTCTCCTGTGCAGCCTCTGGATTCACGTTTAGTAGCTAT |
| | heavy | heavy chain | | TGGATGAGTTGGGTCCGCCAGGCTCCAGGGAAGGGGCTGGAGTGGGTG |
| | chain | | | GCCAACATCAAAGAAGATGGAAGTGAGAAATACTATGTCGACTCTGTG |
| | sequence | | | AAGGGCCGATTCACCATCTCCAGAGACAACGCCAAGAACTCACTGTAT |
| | | | | CTGCAAATGAACAGCCTGAGAGCCGAGGACACGTCTGTGTATTACTGT |
| | | | | GCGAGAAATCGACTCTACAGTGACTTCCTTGACAACTGGGGCCAGGGA |
| | | | | ACCCTGGTCACCGTCTCCTCAGCCAGCACCAAGGGCCCCTCTGTGTTC |
| | | | | CCTCTGGCCCCTTCCAGCAAGTCCACCTCTGGCGGAACAGCCGCTCTG |
| | | | | GGCTGCCTCGTGAAGGACTACTTCCCCGAGCCTGTGACCGTGTCCTGG |
| | | | | AACTCTGGCGCTCTGACCAGCGGAGTGCACACCTTCCCTGCTGTGCTG |
| | | | | CAGTCCTCCGGCCTGTACTCCCTGTCCTCCGTCGTGACCGTGCCTTCC |
| | | | | AGCTCTCTGGGCACCCAGACCTACATCTGCAACGTGAACCACAAGCCC |
| | | | | TCCAACACCAAGGTGGACAAGAAGGTGGAACCCAAGTCCTGCGACAAG |
| | | | | ACCCACACCTGTCCCCCTTGTCCTGCCCCTGAACTGCTGGGCGGACCT |
| | | | | TCCGTGTTCCTGTTCCCCCCAAAGCCCAAGGACACCCTGATGATCTCC |
| | | | | CGGACCCCCGAAGTGACCTGCGTGGTGGTGGATGTGTCCCACGAGGAC |
| | | | | CCTGAAGTGAAGTTCAATTGGTACGTGGACGGCGTGGAAGTGCACAAC |
| | | | | GCCAAGACCAAGCCTAGAGAGGAACAGTACAACTCCACCTACCGGGTG |
| | | | | GTGTCCGTGCTGACCGTGCTGCACCAGGATTGGCTGAACGGCAAAGAG |
| | | | | TACAAGTGCAAGGTGTCCAACAAGGCCCTGCCTGCCCCCATCGAAAAG |
| | | | | ACCATCTCCAAGGCCAAGGGCCAGCCCCGGGAACCCCAGGTGTACACA |
| | | | | CTGCCCCCTAGCAGGGACGAGCTGACCAAGAACCAGGTGTCCCTGACC |
| | | | | TGTCTCGTGAAAGGCTTCTACCCCTCCGATATCGCCGTGGAATGGGAG |
| | | | | TCCAACGGCCAGCCTGAGAACAACTACAAGACCACCCCCCCTGTGCTG |
| | | | | GACTCCGACGGCTCATTCTTCCTGTACAGCAAGCTGACAGTGGACAAG |
| | | | | TCCCGGTGGCAGCAGGGCAACGTGTTCTCCTGCTCCGTGATGCACGAG |
| | | | | GCCCTGCACAACCACTACACCCAGAAGTCCCTGTCCCTGAGCCCCGGC |
| | | | | AAG |
| 62 | 411B08- | Amino acid sequence | | QGVSSW |
| | CDRL1 | of CDRL1 of 411B08 | | |
| | (IMGT) | using IMGT | | |
| 63 | 411B08- | Amino acid sequence | | GAS |
| | CDRL2 | of CDRL2 of 411B08 | | |
| | (IMGT) | using IMGT | | |
| 64 | 411B08- | Amino acid sequence | | QQANSIPFT |
| | CDRL3 | of CDRL3 of 411B08 | | |
| | (IMGT) | using IMGT | | |
| 65 | 411B08- | Amino acid sequence | | RASQGVSSWLA |
| | CDRL1 | of CDRL1 of 411B08 | | |
| | (Kabat) | using Kabat | | |
| 66 | 411B08- | Amino acid sequence | | GASSLQS |
| | CDRL2 | of CDRL2 of 411B08 | | |
| | (Kabat) | using Kabat | | |
| 67 | 411B08- | Amino acid sequence | | QQANSIPFT |
| | CDRL3 | of CDRL3 of 411B08 | | |
| | (Kabat) | using Kabat | | |
| 68 | 411B08- | Amino acid sequence | | DIQMTQSPSSVSASVGDRVTITCRASQGVSSWLAWYQQKSGKAPKLLI |
| | Light | of V_{L} of 411B08 | | YGASSLQSGVPSRFSGSGSGTEFILTISSLQPEDFATYYCQQANSIPF |
| | chain | | | TFGPGTKVDIK |
| | variable | | | |
| | region | | | |
| 69 | 411B08- | Nucleic acid | | GACATCCAGATGACCCAGTCTCCATCTTCCGTGTCTGCATCTGTCGGA |
| | Light | sequence of V_{L} of | | GACAGAGTCACCATCACTTGTCGGGCGAGTCAGGGTGTTAGCAGCTGG |
| | chain | 411B08 | | TTAGCCTGGTATCAGCAGAAATCAGGGAAAGCCCCTAAGCTCCTGATC |
| | variable | | | TATGGTGCATCCAGTTTGCAAAGTGGGGTCCCATCAAGATTCAGCGGC |
| | region | | | AGTGGATCTGGGACAGAGTTCATTCTCACCATCAGCAGCCTGCAGCCT |
| | | | | GAAGATTTTGCAACTTACTATTGTCAACAGGCTAACAGTATCCCATTC |
| | | | | ACTTTCGGCCCTGGGACCAAAGTGGATATCAAAC |
| 70 | 411B08- | Amino acid sequence | | DIQMTQSPSSVSASVGDRVTITCRASQGVSSWLAWYQQKSGKAPKLLI |
| | full | of 411B08 light | | YGASSLQSGVPSRFSGSGSGTEFILTISSLQPEDFATYYCQQANSIPF |
| | light | chain | | TFGPGTKVDIKRTVAAPSVFIFPPSDEQLKSGTASVVCLLNNFYPREA |
| | chain | | | KVQWKVDNALQSGNSQESVTEQDSKDSTYSLSSTLTLSKADYEKHKVY |
| | sequence | | | ACEVTHQGLSSPVTKSFNRGEC |
| 71 | 411B08- | Nucleic acid | | GACATCCAGATGACCCAGTCTCCATCTTCCGTGTCTGCATCTGTCGGA |
| | full | sequence of 411B08 | | GACAGAGTCACCATCACTTGTCGGGCGAGTCAGGGTGTTAGCAGCTGG |
| | light | light chain | | TTAGCCTGGTATCAGCAGAAATCAGGGAAAGCCCCTAAGCTCCTGATC |
| | chain | | | TATGGTGCATCCAGTTTGCAAAGTGGGGTCCCATCAAGATTCAGCGGC |
| | sequence | | | AGTGGATCTGGGACAGAGTTCATTCTCACCATCAGCAGCCTGCAGCCT |
| | | | | GAAGATTTTGCAACTTACTATTGTCAACAGGCTAACAGTATCCCATTC |
| | | | | ACTTTCGGCCCTGGGACCAAAGTGGATATCAAACGTACGGTGGCCGCT |
| | | | | CCCTCCGTGTTCATCTTCCCACCTTCCGACGAGCAGCTGAAGTCCGGC |
| | | | | ACCGCTTCTGTCGTGTGCCTGCTGAACAACTTCTACCCCCGCGAGGCC |
| | | | | AAGGTGCAGTGGAAGGTGGACAACGCCCTGCAGTCCGGCAACTCCCAG |
| | | | | GAATCCGTGACCGAGCAGGACTCCAAGGACAGCACCTACTCCCTGTCC |
| | | | | TCCACCCTGACCCTGTCCAAGGCCGACTACGAGAAGCACAAGGTGTAC |
| | | | | GCCTGCGAAGTGACCCACCAGGGCCTGTCTAGCCCCGTGACCAAGTCT |
| | | | | TTCAACCGGGGCGAGTGT |
| 72 | 411C04- | Amino acid sequence | | GFTFSSYW |
| | CDRH1 | of CDRH1 of 411C04 | | |
| | (IMGT) | using IMGT | | |
| 73 | 411C04- | Amino acid sequence | | IKEDGSEK |
| | CDRH2 | of CDRH2 of 411C04 | | |
| | IMGT) | using IMGT | | |
| 74 | 411C04- | Amino acid sequence | | ARVRLYSDFLDY |
| | CDRH3 | of CDRH3 of 411C04 | | |
| | (IMGT) | using IMGT | | |
| 75 | 411C04- | Amino acid sequence | | SYWMS |
| | CDRH1 | of CDRH1 of 411C04 | | |
| | (Kabat) | using Kabat | | |
| 76 | 411C04- | Amino acid sequence | | NIKEDGSEKYYVDSLKG |
| | CDRH2 | of CDRH2 of 411C04 | | |
| | (Kabat) | using Kabat | | |
| 77 | 411C04- | Amino acid sequence | | VRLYSDELDY |
| | CDRH3 | of CDRH3 of 411C04 | | |
| | (Kabat) | using Kabat | | |
| 78 | 411C04- | Amino acid sequence | | EVQLVDSGGGLVQPGGSLRLSCAASGFTFSSYWMSWVRQAPGKGLEWV |
| | Heavy | of V_{H} of 411C04 | | ANIKEDGSEKYYVDSLKGRFTISRDNAKNSLYLQMNSLRAEDTSVYYC |
| | chain | | | ARVRLYSDFLDYWGQGTLVTVSS |
| | variable | | | |
| | region | | | |
| 79 | 411C04- | Nucleic acid | | GAGGTGCAGCTGGTGGACTCTGGGGGAGGCTTGGTCCAGCCTGGGGGG |
| | Heavy | sequence of V_{H} of | | TCCCTGAGACTCTCCTGTGCAGCCTCTGGATTCACGTTTAGTAGCTAT |
| | chain | 411C04 | | TGGATGAGTTGGGTCCGCCAGGCTCCAGGAAAGGGGCTGGAGTGGGTG |
| | variable | | | GCCAACATAAAAGAAGATGGAAGTGAGAAATACTATGTAGACTCTTTG |
| | region | | | AAGGGCCGATTCACCATCTCCAGAGACAACGCCAAGAACTCACTGTAT |
| | | | | CTGCAAATGAACAGCCTGAGAGCCGAGGACACGTCTGTGTATTACTGT |
| | | | | GCGAGAGTTCGACTCTACAGTGACTTCCTTGACTACTGGGGCCAGGGA |
| | | | | ACCCTGGTCACCGTCTCCTCAG |
| 80 | 411C04- | Amino acid sequence | | EVQLVDSGGGLVQPGGSLRLSCAASGFTFSSYWMSWVRQAPGKGLEWV |
| | full | of 411C04 heavy | | ANIKEDGSEKYYVDSLKGRFTISRDNAKNSLYLQMNSLRAEDTSVYYC |
| | heavy | chain | | ARVRLYSDFLDYWGQGTLVTVSSASTKGPSVFPLAPSSKSTSGGTAAL |
| | chain | | | GCLVKDYFPEPVTVSWNSGALTSGVHTFPAVLQSSGLYSLSSVVTVPS |
| | sequence | | | SSLGTQTYICNVNHKPSNTKVDKKVEPKSCDKTHTCPPCPAPELLGGP |
| | | | | SVFLFPPKPKDTLMISRTPEVTCVVVDVSHEDPEVKFNWYVDGVEVHN |
| | | | | AKTKPREEQYNSTYRVVSVLTVLHQDWLNGKEYKCKVSNKALPAPIEK |
| | | | | TISKAKGQPREPQVYTLPPSRDELTKNQVSLTCLVKGFYPSDIAVEWE |
| | | | | SNGQPENNYKTTPPVLDSDGSFFLYSKLTVDKSRWQQGNVFSCSVMHE |
| | | | | ALHNHYTQKSLSLSPGK |
| 81 | 411C04- | Nucleic acid | | GAGGTGCAGCTGGTGGACTCTGGGGGAGGCTTGGTCCAGCCTGGGGGG |
| | full | sequence of 411C04 | | TCCCTGAGACTCTCCTGTGCAGCCTCTGGATTCACGTTTAGTAGCTAT |
| | heavy | heavy chain | | TGGATGAGTTGGGTCCGCCAGGCTCCAGGAAAGGGGCTGGAGTGGGTG |
| | chain | | | GCCAACATAAAAGAAGATGGAAGTGAGAAATACTATGTAGACTCTTTG |
| | sequence | | | AAGGGCCGATTCACCATCTCCAGAGACAACGCCAAGAACTCACTGTAT |
| | | | | CTGCAAATGAACAGCCTGAGAGCCGAGGACACGTCTGTGTATTACTGT |
| | | | | GCGAGAGTTCGACTCTACAGTGACTTCCTTGACTACTGGGGCCAGGGA |
| | | | | ACCCTGGTCACCGTCTCCTCAGCCAGCACCAAGGGCCCCTCTGTGTTC |
| | | | | CCTCTGGCCCCTTCCAGCAAGTCCACCTCTGGCGGAACAGCCGCTCTG |
| | | | | GGCTGCCTCGTGAAGGACTACTTCCCCGAGCCTGTGACCGTGTCCTGG |
| | | | | AACTCTGGCGCTCTGACCAGCGGAGTGCACACCTTCCCTGCTGTGCTG |
| | | | | CAGTCCTCCGGCCTGTACTCCCTGTCCTCCGTCGTGACCGTGCCTTCC |
| | | | | AGCTCTCTGGGCACCCAGACCTACATCTGCAACGTGAACCACAAGCCC |
| | | | | TCCAACACCAAGGTGGACAAGAAGGTGGAACCCAAGTCCTGCGACAAG |
| | | | | ACCCACACCTGTCCCCCTTGTCCTGCCCCTGAACTGCTGGGCGGACCT |
| | | | | TCCGTGTTCCTGTTCCCCCCAAAGCCCAAGGACACCCTGATGATCTCC |
| | | | | CGGACCCCCGAAGTGACCTGCGTGGTGGTGGATGTGTCCCACGAGGAC |
| | | | | CCTGAAGTGAAGTTCAATTGGTACGTGGACGGCGTGGAAGTGCACAAC |
| | | | | GCCAAGACCAAGCCTAGAGAGGAACAGTACAACTCCACCTACCGGGTG |
| | | | | GTGTCCGTGCTGACCGTGCTGCACCAGGATTGGCTGAACGGCAAAGAG |
| | | | | TACAAGTGCAAGGTGTCCAACAAGGCCCTGCCTGCCCCCATCGAAAAG |
| | | | | ACCATCTCCAAGGCCAAGGGCCAGCCCCGGGAACCCCAGGTGTACACA |
| | | | | CTGCCCCCTAGCAGGGACGAGCTGACCAAGAACCAGGTGTCCCTGACC |
| | | | | TGTCTCGTGAAAGGCTTCTACCCCTCCGATATCGCCGTGGAATGGGAG |
| | | | | TCCAACGGCCAGCCTGAGAACAACTACAAGACCACCCCCCCTGTGCTG |
| | | | | GACTCCGACGGCTCATTCTTCCTGTACAGCAAGCTGACAGTGGACAAG |
| | | | | TCCCGGTGGCAGCAGGGCAACGTGTTCTCCTGCTCCGTGATGCACGAG |
| | | | | GCCCTGCACAACCACTACACCCAGAAGTCCCTGTCCCTGAGCCCCGGC |
| | | | | AAG |
| 82 | 411C04- | Amino acid sequence | | QGVSSW |
| | CDRL1 | of CDRL1 of 411C04 | | |
| | (IMGT) | using IMGT | | |
| 83 | 411C04- | Amino acid sequence | | GAS |
| | CDRL2 | of CDRL2 of 411C04 | | |
| | (IMGT) | using IMGT | | |
| 84 | 411C04- | Amino acid sequence | | QQANSIPFT |
| | CDRL3 | of CDRL3 of 411C04 | | |
| | (IMGT) | using IMGT | | |
| 85 | 411C04- | Amino acid sequence | | RASQGVSSWLA |
| | CDRL1 | of CDRL1 of 411C04 | | |
| | (Kabat) | using Kabat | | |
| 86 | 411C04- | Amino acid sequence | | GASSLQS |
| | CDRL2 | of CDRL2 of 411C04 | | |
| | (Kabat) | using Kabat | | |
| 87 | 411C04- | Amino acid sequence | | QQANSIPFT |
| | CDRL3 | of CDRL3 of 411C04 | | |
| | (Kabat) | using Kabat | | |
| 88 | 411C04- | Amino acid sequence | | DIQMTQSPSSVSASVGDRVTITCRASQGVSSWLAWYQQKSGKAPKLLI |
| | Light | of V_{L} of 411C04 | | YGASSLQSGVPSRFSGSGSGTEFILSISSLQPEDFATYYCQQANSIPF |
| | chain | | | TFGPGTKVDIK |
| | variable | | | |
| | region | | | |
| 89 | 411C04- | Nucleic acid | | GACATCCAGATGACCCAGTCTCCATCTTCCGTGTCTGCATCTGTCGGA |
| | Light | sequence of V_{L} of | | GACAGAGTCACCATCACTTGTCGGGCGAGTCAGGGTGTTAGCAGTTGG |
| | chain | 411C04 | | TTAGCCTGGTATCAGCAGAAATCAGGGAAAGCCCCTAAGCTCCTGATC |
| | variable | | | TATGGTGCCTCCAGTTTGCAAAGTGGGGTCCCATCAAGATTCAGCGGC |
| | region | | | AGTGGATCTGGGACAGAGTTCATTCTCAGCATCAGCAGCCTGCAGCCT |
| | | | | GAAGATTTTGCAACTTACTATTGTCAACAGGCTAACAGTATCCCATTC |
| | | | | ACTTTCGGCCCTGGGACCAAAGTGGATATCAAAC |
| 90 | 411C04- | Amino acid sequence | | DIQMTQSPSSVSASVGDRVTITCRASQGVSSWLAWYQQKSGKAPKLLI |
| | full | of 411C04 light | | YGASSLQSGVPSRFSGSGSGTEFILSISSLQPEDFATYYCQQANSIPF |
| | light | chain | | TFGPGTKVDIKRTVAAPSVFIFPPSDEQLKSGTASVVCLLNNFYPREA |
| | chain | | | KVQWKVDNALQSGNSQESVTEQDSKDSTYSLSSTLTLSKADYEKHKVY |
| | sequence | | | ACEVTHQGLSSPVTKSFNRGEC |
| 91 | 411C04- | Nucleic acid | | GACATCCAGATGACCCAGTCTCCATCTTCCGTGTCTGCATCTGTCGGA |
| | full | sequence of 411C04 | | GACAGAGTCACCATCACTTGTCGGGCGAGTCAGGGTGTTAGCAGTTGG |
| | light | light chain | | TTAGCCTGGTATCAGCAGAAATCAGGGAAAGCCCCTAAGCTCCTGATC |
| | chain | | | TATGGTGCCTCCAGTTTGCAAAGTGGGGTCCCATCAAGATTCAGCGGC |
| | sequence | | | AGTGGATCTGGGACAGAGTTCATTCTCAGCATCAGCAGCCTGCAGCCT |
| | | | | GAAGATTTTGCAACTTACTATTGTCAACAGGCTAACAGTATCCCATTC |
| | | | | ACTTTCGGCCCTGGGACCAAAGTGGATATCAAACGTACGGTGGCCGCT |
| | | | | CCCTCCGTGTTCATCTTCCCACCTTCCGACGAGCAGCTGAAGTCCGGC |
| | | | | ACCGCTTCTGTCGTGTGCCTGCTGAACAACTTCTACCCCCGCGAGGCC |
| | | | | AAGGTGCAGTGGAAGGTGGACAACGCCCTGCAGTCCGGCAACTCCCAG |
| | | | | GAATCCGTGACCGAGCAGGACTCCAAGGACAGCACCTACTCCCTGTCC |
| | | | | TCCACCCTGACCCTGTCCAAGGCCGACTACGAGAAGCACAAGGTGTAC |
| | | | | GCCTGCGAAGTGACCCACCAGGGCCTGTCTAGCCCCGTGACCAAGTCT |
| | | | | TTCAACCGGGGCGAGTGT |
| 92 | 411D07- | Amino acid sequence | | GGSIISSDW |
| | CDRH1 | of CDRH1 of 411D07 | | |
| | (IMGT) | using IMGT | | |
| 93 | 411D07- | Amino acid sequence | | IFHSGRT |
| | CDRH2 | of CDRH2 of 411D07 | | |
| | (IMGT) | using IMGT | | |
| 94 | 411D07- | Amino acid sequence | | ARDGSGSY |
| | CDRH3 | of CDRH3 of 411D07 | | |
| | (IMGT) | using IMGT | | |
| 95 | 411D07- | Amino acid sequence | | SSDWWN |
| | CDRH1 | of CDRH1 of 411D07 | | |
| | (Kabat) | using Kabat | | |
| 96 | 411D07- | Amino acid sequence | | EIFHSGRTNYNPSLKS |
| | CDRH2 | of CDRH2 of 411D07 | | |
| | (Kabat) | using Kabat | | |
| 97 | 411D07- | Amino acid sequence | | DGSGSY |
| | CDRH3 | of CDRH3 of 411D07 | | |
| | (Kabat) | using Kabat | | |
| 98 | 411D07- | Amino acid sequence | | QVQLQESGPGLVKPSGTLSLTCIVSGGSIISSDWWNWVRQPPGKGLEW |
| | Heavy | of V_{H} of 411D07 | | IGEIFHSGRTNYNPSLKSRVTISIDKSKNQFSLRLSSVTAADTAVYYC |
| | chain | | | ARDGSGSYWGQGTLVTVSS |
| | variable | | | |
| | region | | | |
| 99 | 411D07- | Nucleic acid | | CAGGTGCAGCTGCAGGAGTCGGGCCCAGGACTGGTGAAGCCTTCGGGG |
| | Heavy | sequence of V_{H} of | | ACCCTGTCCCTCACCTGCATTGTCTCTGGTGGCTCCATCATCAGTAGT |
| | chain | 411D07 | | GACTGGTGGAATTGGGTCCGCCAGCCCCCAGGGAAGGGGCTGGAGTGG |
| | variable | | | ATTGGAGAAATCTTTCATAGTGGGAGGACCAACTACAACCCGTCCCTC |
| | region | | | AAGAGTCGAGTCACCATATCAATAGACAAGTCCAAGAATCAGTTCTCC |
| | | | | CTGAGGCTGAGCTCTGTGACCGCCGCGGACACGGCCGTGTATTACTGT |
| | | | | GCGAGAGATGGTTCGGGGAGTTACTGGGGCCAGGGAACCCTGGTCACC |
| | | | | GTCTCCTCAG |
| 100 | 411D07- | Amino acid sequence | | QVQLQESGPGLVKPSGTLSLTCIVSGGSIISSDWWNWVRQPPGKGLEW |
| | full | of 411D07 heavy | | IGEIFHSGRTNYNPSLKSRVTISIDKSKNQFSLRLSSVTAADTAVYYC |
| | heavy | chain | | ARDGSGSYWGQGTLVTVSSASTKGPSVFPLAPSSKSTSGGTAALGCLV |
| | chain | | | KDYFPEPVTVSWNSGALTSGVHTFPAVLQSSGLYSLSSVVTVPSSSLG |
| | sequence | | | TQTYICNVNHKPSNTKVDKKVEPKSCDKTHTCPPCPAPELLGGPSVEL |
| | | | | FPPKPKDTLMISRTPEVTCVVVDVSHEDPEVKFNWYVDGVEVHNAKTK |
| | | | | PREEQYNSTYRVVSVLTVLHQDWLNGKEYKCKVSNKALPAPIEKTISK |
| | | | | AKGQPREPQVYTLPPSRDELTKNQVSLTCLVKGFYPSDIAVEWESNGQ |
| | | | | PENNYKTTPPVLDSDGSFFLYSKLTVDKSRWQQGNVFSCSVMHEALHN |
| | | | | HYTQKSLSLSPGK |
| 101 | 411D07- | Nucleic acid | | CAGGTGCAGCTGCAGGAGTCGGGCCCAGGACTGGTGAAGCCTTCGGGG |
| | full | sequence of 411D07 | | ACCCTGTCCCTCACCTGCATTGTCTCTGGTGGCTCCATCATCAGTAGT |
| | heavy | heavy chain | | GACTGGTGGAATTGGGTCCGCCAGCCCCCAGGGAAGGGGCTGGAGTGG |
| | chain | | | ATTGGAGAAATCTTTCATAGTGGGAGGACCAACTACAACCCGTCCCTC |
| | sequence | | | AAGAGTCGAGTCACCATATCAATAGACAAGTCCAAGAATCAGTTCTCC |
| | | | | CTGAGGCTGAGCTCTGTGACCGCCGCGGACACGGCCGTGTATTACTGT |
| | | | | GCGAGAGATGGTTCGGGGAGTTACTGGGGCCAGGGAACCCTGGTCACC |
| | | | | GTCTCCTCAGCCAGCACCAAGGGCCCCTCTGTGTTCCCTCTGGCCCCT |
| | | | | TCCAGCAAGTCCACCTCTGGCGGAACAGCCGCTCTGGGCTGCCTCGTG |
| | | | | AAGGACTACTTCCCCGAGCCTGTGACCGTGTCCTGGAACTCTGGCGCT |
| | | | | CTGACCAGCGGAGTGCACACCTTCCCTGCTGTGCTGCAGTCCTCCGGC |
| | | | | CTGTACTCCCTGTCCTCCGTCGTGACCGTGCCTTCCAGCTCTCTGGGC |
| | | | | ACCCAGACCTACATCTGCAACGTGAACCACAAGCCCTCCAACACCAAG |
| | | | | GTGGACAAGAAGGTGGAACCCAAGTCCTGCGACAAGACCCACACCTGT |
| | | | | CCCCCTTGTCCTGCCCCTGAACTGCTGGGCGGACCTTCCGTGTTCCTG |
| | | | | TTCCCCCCAAAGCCCAAGGACACCCTGATGATCTCCCGGACCCCCGAA |
| | | | | GTGACCTGCGTGGTGGTGGATGTGTCCCACGAGGACCCTGAAGTGAAG |
| | | | | TTCAATTGGTACGTGGACGGCGTGGAAGTGCACAACGCCAAGACCAAG |
| | | | | CCTAGAGAGGAACAGTACAACTCCACCTACCGGGTGGTGTCCGTGCTG |
| | | | | ACCGTGCTGCACCAGGATTGGCTGAACGGCAAAGAGTACAAGTGCAAG |
| | | | | GTGTCCAACAAGGCCCTGCCTGCCCCCATCGAAAAGACCATCTCCAAG |
| | | | | GCCAAGGGCCAGCCCCGGGAACCCCAGGTGTACACACTGCCCCCTAGC |
| | | | | AGGGACGAGCTGACCAAGAACCAGGTGTCCCTGACCTGTCTCGTGAAA |
| | | | | GGCTTCTACCCCTCCGATATCGCCGTGGAATGGGAGTCCAACGGCCAG |
| | | | | CCTGAGAACAACTACAAGACCACCCCCCCTGTGCTGGACTCCGACGGC |
| | | | | TCATTCTTCCTGTACAGCAAGCTGACAGTGGACAAGTCCCGGTGGCAG |
| | | | | CAGGGCAACGTGTTCTCCTGCTCCGTGATGCACGAGGCCCTGCACAAC |
| | | | | CACTACACCCAGAAGTCCCTGTCCCTGAGCCCCGGCAAG |
| 102 | 411D07- | Amino acid sequence | | QSVLYSSNNKNY |
| | CDRL1 | of CDRL1 of 411D07 | | |
| | (IMGT) | using IMGT | | |
| 103 | 411D07- | Amino acid sequence | | WAS |
| | CDRL2 | of CDRL2 of 411D07 | | |
| | (IMGT) | using IMGT | | |
| 104 | 411D07- | Amino acid sequence | | QQYYSNRS |
| | CDRL3 | of CDRL3 of 411D07 | | |
| | (IMGT) | using IMGT | | |
| 105 | 411D07- | Amino acid sequence | | KSSQSVLYSSNNKNYLA |
| | CDRL1 | of CDRL1 of 411D07 | | |
| | (Kabat) | using Kabat | | |
| 106 | 411D07- | Amino acid sequence | | WASTRES |
| | CDRL2 | of CDRL2 of 411D07 | | |
| | (Kabat) | using Kabat | | |
| 107 | 411D07- | Amino acid sequence | | QQYYSNRS |
| | CDRL3 | of CDRL3 of 411D07 | | |
| | (Kabat) | using Kabat | | |
| 108 | 411D07- | Amino acid sequence | | DIVMTQSPDSLAVSLGERATINCKSSQSVLYSSNNKNYLAWYQQKSGQ |
| | Light | of V_{L} of 411D07 | | PPKLLIYWASTRESGVPDRFSGSGSGTDFTLTISSLQTEDVAVYYCQQ |
| | chain | | | YYSNRSFGQGTKLEIK |
| | variable | | | |
| | region | | | |
| 109 | 411D07- | Nucleic acid | | GACATCGTGATGACCCAGTCTCCAGACTCCCTGGCTGTGTCTCTGGGC |
| | Light | sequence of V_{L} of | | GAGAGGGCCACCATCAACTGCAAGTCCAGCCAGAGTGTTTTATACAGC |
| | chain | 411D07 | | TCCAACAATAAGAATTACTTAGCTTGGTACCAGCAGAAATCAGGACAG |
| | variable | | | CCTCCTAAGTTGCTCATTTACTGGGCATCTACCCGGGAATCCGGGGTC |
| | region | | | CCTGACCGATTCAGTGGCAGCGGGTCTGGGACAGATTTCACTCTCACC |
| | | | | ATCAGCAGCCTGCAGACTGAAGATGTGGCAGTTTATTACTGTCAGCAA |
| | | | | TATTATAGTAATCGCAGTTTTGGCCAGGGGACCAAGCTGGAGATCAAA |
| | | | | C |
| 110 | 411D07- | Amino acid sequence | | DIVMTQSPDSLAVSLGERATINCKSSQSVLYSSNNKNYLAWYQQKSGQ |
| | full | of 411D07 light | | PPKLLIYWASTRESGVPDRFSGSGSGTDFTLTISSLQTEDVAVYYCQQ |
| | light | chain | | YYSNRSFGQGTKLEIKRTVAAPSVFIFPPSDEQLKSGTASVVCLLNNE |
| | chain | | | YPREAKVQWKVDNALQSGNSQESVTEQDSKDSTYSLSSTLTLSKADYE |
| | sequence | | | KHKVYACEVTHQGLSSPVTKSFNRGEC |
| 111 | 411D07- | Nucleic acid | | GACATCGTGATGACCCAGTCTCCAGACTCCCTGGCTGTGTCTCTGGGC |
| | full | sequence of 411D07 | | GAGAGGGCCACCATCAACTGCAAGTCCAGCCAGAGTGTTTTATACAGC |
| | light | light chain | | TCCAACAATAAGAATTACTTAGCTTGGTACCAGCAGAAATCAGGACAG |
| | chain | | | CCTCCTAAGTTGCTCATTTACTGGGCATCTACCCGGGAATCCGGGGTC |
| | sequence | | | CCTGACCGATTCAGTGGCAGCGGGTCTGGGACAGATTTCACTCTCACC |
| | | | | ATCAGCAGCCTGCAGACTGAAGATGTGGCAGTTTATTACTGTCAGCAA |
| | | | | TATTATAGTAATCGCAGTTTTGGCCAGGGGACCAAGCTGGAGATCAAA |
| | | | | CGTACGGTGGCCGCTCCCTCCGTGTTCATCTTCCCACCTTCCGACGAG |
| | | | | CAGCTGAAGTCCGGCACCGCTTCTGTCGTGTGCCTGCTGAACAACTTC |
| | | | | TACCCCCGCGAGGCCAAGGTGCAGTGGAAGGTGGACAACGCCCTGCAG |
| | | | | TCCGGCAACTCCCAGGAATCCGTGACCGAGCAGGACTCCAAGGACAGC |
| | | | | ACCTACTCCCTGTCCTCCACCCTGACCCTGTCCAAGGCCGACTACGAG |
| | | | | AAGCACAAGGTGTACGCCTGCGAAGTGACCCACCAGGGCCTGTCTAGC |
| | | | | CCCGTGACCAAGTCTTTCAACCGGGGCGAGTGT |
| 112 | 385F01- | Amino acid sequence | | GFTFSSYW |
| | CDRH1 | of CDRH1 of 385F01 | | |
| | (IMGT) | using IMGT | | |
| 113 | 385F01- | Amino acid sequence | | IKEDGSEK |
| | CDRH2 | of CDRH2 of 385F01 | | |
| | (IMGT) | using IMGT | | |
| 114 | 385F01- | Amino acid sequence | | ARNRLYSDFLDN |
| | CDRH3 | of CDRH3 of 385F01 | | |
| | (IMGT) | using IMGT | | |
| 115 | 385F01- | Amino acid sequence | | SYWMS |
| | CDRH1 | of CDRH1 of 385F01 | | |
| | (Kabat) | using Kabat | | |
| 116 | 385F01- | Amino acid sequence | | NIKEDGSEKYYVDSVKG |
| | CDRH2 | of CDRH2 of 385F01 | | |
| | (Kabat) | using Kabat | | |
| 117 | 385F01- | Amino acid sequence | | NRLYSDELDN |
| | CDRH3 | of CDRH3 of 385F01 | | |
| | (Kabat) | using Kabat | | |
| 118 | 385F01- | Amino acid sequence | | EVQLVESGGGLVQPGGSLRLSCAASGFTFSSYWMSWVRQAPGKGLEWV |
| | Heavy | of V_{H} of 385F01 | | ANIKEDGSEKYYVDSVKGRFTISRDNAKNSLYLQMNSLRAEDTSVYYC |
| | chain | | | ARNRLYSDFLDNWGQGTLVTVSS |
| | variable | | | |
| | region | | | |
| 119 | 385F01- | Nucleic acid | | GAGGTGCAGCTGGTGGAGTCTGGGGGAGGCTTGGTCCAGCCTGGGGGG |
| | Heavy | sequence of V_{H} of | | TCCCTGAGACTCTCCTGTGCAGCCTCTGGATTCACGTTTAGTAGCTAT |
| | chain | 385F01 | | TGGATGAGTTGGGTCCGCCAGGCTCCAGGGAAGGGGCTGGAGTGGGTG |
| | variable | | | GCCAACATCAAAGAAGATGGAAGTGAGAAATACTATGTCGACTCTGTG |
| | region | | | AAGGGCCGATTCACCATCTCCAGAGACAACGCCAAGAACTCACTGTAT |
| | | | | CTGCAAATGAACAGCCTGAGAGCCGAGGACACGTCTGTGTATTACTGT |
| | | | | GCGAGAAATCGACTCTACAGTGACTTCCTTGACAACTGGGGCCAGGGA |
| | | | | ACCCTGGTCACCGTCTCCTCAG |
| 120 | 385F01- | Amino acid sequence | | EVQLVESGGGLVQPGGSLRLSCAASGFTFSSYWMSWVRQAPGKGLEWV |
| | full | of 385F01 heavy | | ANIKEDGSEKYYVDSVKGRFTISRDNAKNSLYLQMNSLRAEDTSVYYC |
| | heavy | chain | | ARNRLYSDFLDNWGQGTLVTVSSASTKGPSVFPLAPSSKSTSGGTAAL |
| | chain | | | GCLVKDYFPEPVTVSWNSGALTSGVHTFPAVLQSSGLYSLSSVVTVPS |
| | sequence | | | SSLGTQTYICNVNHKPSNTKVDKKVEPKSCDKTHTCPPCPAPELLGGP |
| | | | | SVFLFPPKPKDTLMISRTPEVTCVVVDVSHEDPEVKFNWYVDGVEVHN |
| | | | | AKTKPREEQYNSTYRVVSVLTVLHQDWLNGKEYKCKVSNKALPAPIEK |
| | | | | TISKAKGQPREPQVYTLPPSRDELTKNQVSLTCLVKGFYPSDIAVEWE |
| | | | | SNGQPENNYKTTPPVLDSDGSFFLYSKLTVDKSRWQQGNVFSCSVMHE |
| | | | | ALHNHYTQKSLSLSPGK |
| 121 | 385F01- | Nucleic acid | | GAGGTGCAGCTGGTGGAGTCTGGGGGAGGCTTGGTCCAGCCTGGGGGG |
| | full | sequence of 385F01 | | TCCCTGAGACTCTCCTGTGCAGCCTCTGGATTCACGTTTAGTAGCTAT |
| | heavy | heavy chain | | TGGATGAGTTGGGTCCGCCAGGCTCCAGGGAAGGGGCTGGAGTGGGTG |
| | chain | | | GCCAACATCAAAGAAGATGGAAGTGAGAAATACTATGTCGACTCTGTG |
| | sequence | | | AAGGGCCGATTCACCATCTCCAGAGACAACGCCAAGAACTCACTGTAT |
| | | | | CTGCAAATGAACAGCCTGAGAGCCGAGGACACGTCTGTGTATTACTGT |
| | | | | GCGAGAAATCGACTCTACAGTGACTTCCTTGACAACTGGGGCCAGGGA |
| | | | | ACCCTGGTCACCGTCTCCTCAGCCAGCACCAAGGGCCCCTCTGTGTTC |
| | | | | CCTCTGGCCCCTTCCAGCAAGTCCACCTCTGGCGGAACAGCCGCTCTG |
| | | | | GGCTGCCTCGTGAAGGACTACTTCCCCGAGCCTGTGACCGTGTCCTGG |
| | | | | AACTCTGGCGCTCTGACCAGCGGAGTGCACACCTTCCCTGCTGTGCTG |
| | | | | CAGTCCTCCGGCCTGTACTCCCTGTCCTCCGTCGTGACCGTGCCTTCC |
| | | | | AGCTCTCTGGGCACCCAGACCTACATCTGCAACGTGAACCACAAGCCC |
| | | | | TCCAACACCAAGGTGGACAAGAAGGTGGAACCCAAGTCCTGCGACAAG |
| | | | | ACCCACACCTGTCCCCCTTGTCCTGCCCCTGAACTGCTGGGCGGACCT |
| | | | | TCCGTGTTCCTGTTCCCCCCAAAGCCCAAGGACACCCTGATGATCTCC |
| | | | | CGGACCCCCGAAGTGACCTGCGTGGTGGTGGATGTGTCCCACGAGGAC |
| | | | | CCTGAAGTGAAGTTCAATTGGTACGTGGACGGCGTGGAAGTGCACAAC |
| | | | | GCCAAGACCAAGCCTAGAGAGGAACAGTACAACTCCACCTACCGGGTG |
| | | | | GTGTCCGTGCTGACCGTGCTGCACCAGGATTGGCTGAACGGCAAAGAG |
| | | | | TACAAGTGCAAGGTGTCCAACAAGGCCCTGCCTGCCCCCATCGAAAAG |
| | | | | ACCATCTCCAAGGCCAAGGGCCAGCCCCGGGAACCCCAGGTGTACACA |
| | | | | CTGCCCCCTAGCAGGGACGAGCTGACCAAGAACCAGGTGTCCCTGACC |
| | | | | TGTCTCGTGAAAGGCTTCTACCCCTCCGATATCGCCGTGGAATGGGAG |
| | | | | TCCAACGGCCAGCCTGAGAACAACTACAAGACCACCCCCCCTGTGCTG |
| | | | | GACTCCGACGGCTCATTCTTCCTGTACAGCAAGCTGACAGTGGACAAG |
| | | | | TCCCGGTGGCAGCAGGGCAACGTGTTCTCCTGCTCCGTGATGCACGAG |
| | | | | GCCCTGCACAACCACTACACCCAGAAGTCCCTGTCCCTGAGCCCCGGC |
| | | | | AAG |
| 122 | 385F01- | Amino acid sequence | | QGVSSW |
| | CDRL1 | of CDRL1 of 385F01 | | |
| | (IMGT) | using IMGT | | |
| 123 | 385F01- | Amino acid sequence | | GAS |
| | CDRL2 | of CDRL2 of 385F01 | | |
| | (IMGT) | using IMGT | | |
| 124 | 385F01- | Amino acid sequence | | QQANSIPFT |
| | CDRL3 | of CDRL3 of 385F01 | | |
| | (IMGT) | using IMGT | | |
| 125 | 385F01- | Amino acid sequence | | RASQGVSSWLA |
| | CDRL1 | of CDRL1 of 385F01 | | |
| | (Kabat) | using Kabat | | |
| 126 | 385F01- | Amino acid sequence | | GASSLQS |
| | CDRL2 | of CDRL2 of 385F01 | | |
| | (Kabat) | using Kabat | | |
| 127 | 385F01- | Amino acid sequence | | QQANSIPFT |
| | CDRL3 | of CDRL3 of 385F01 | | |
| | (Kabat) | using Kabat | | |
| 128 | 385F01- | Amino acid sequence | | DIQMTQSPSSVSASVGDRVTITCRASQGVSSWLAWYQQKSGKAPKLLI |
| | Light | of V_{L} of 385F01 | | YGASSLQSGVPSRFSGSGSGTEFILTISSLQPEDFATYYCQQANSIPF |
| | chain | | | TFGPGTKVDIK |
| | variable | | | |
| | region | | | |
| 129 | 385F01- | Nucleic acid | | GACATCCAGATGACCCAGTCTCCATCTTCCGTGTCTGCATCTGTCGGA |
| | Light | sequence of V_{L} of | | GACAGAGTCACCATCACTTGTCGGGCGAGTCAGGGTGTTAGCAGCTGG |
| | chain | 385F01 | | TTAGCCTGGTATCAGCAGAAATCAGGGAAAGCCCCTAAGCTCCTGATC |
| | variable | | | TATGGTGCATCCAGTTTGCAAAGTGGGGTCCCATCAAGATTCAGCGGC |
| | region | | | AGTGGATCTGGGACAGAGTTCATTCTCACCATCAGCAGCCTGCAGCCT |
| | | | | GAAGATTTTGCAACTTACTATTGTCAACAGGCTAACAGTATCCCATTC |
| | | | | ACTTTCGGCCCTGGGACCAAAGTGGATATCAAAC |
| 130 | 385F01- | Amino acid sequence | | DIQMTQSPSSVSASVGDRVTITCRASQGVSSWLAWYQQKSGKAPKLLI |
| | full | of 385F01 light | | YGASSLQSGVPSRFSGSGSGTEFILTISSLQPEDFATYYCQQANSIPF |
| | light | chain | | TFGPGTKVDIKRTVAAPSVFIFPPSDEQLKSGTASVVCLLNNFYPREA |
| | chain | | | KVQWKVDNALQSGNSQESVTEQDSKDSTYSLSSTLTLSKADYEKHKVY |
| | sequence | | | ACEVTHQGLSSPVTKSFNRGEC |
| 131 | 385F01- | Nucleic acid | | GACATCCAGATGACCCAGTCTCCATCTTCCGTGTCTGCATCTGTCGGA |
| | full | sequence of 385F01 | | GACAGAGTCACCATCACTTGTCGGGCGAGTCAGGGTGTTAGCAGCTGG |
| | light | light chain | | TTAGCCTGGTATCAGCAGAAATCAGGGAAAGCCCCTAAGCTCCTGATC |
| | chain | | | TATGGTGCATCCAGTTTGCAAAGTGGGGTCCCATCAAGATTCAGCGGC |
| | sequence | | | AGTGGATCTGGGACAGAGTTCATTCTCACCATCAGCAGCCTGCAGCCT |
| | | | | GAAGATTTTGCAACTTACTATTGTCAACAGGCTAACAGTATCCCATTC |
| | | | | ACTTTCGGCCCTGGGACCAAAGTGGATATCAAACGTACGGTGGCCGCT |
| | | | | CCCTCCGTGTTCATCTTCCCACCTTCCGACGAGCAGCTGAAGTCCGGC |
| | | | | ACCGCTTCTGTCGTGTGCCTGCTGAACAACTTCTACCCCCGCGAGGCC |
| | | | | AAGGTGCAGTGGAAGGTGGACAACGCCCTGCAGTCCGGCAACTCCCAG |
| | | | | GAATCCGTGACCGAGCAGGACTCCAAGGACAGCACCTACTCCCTGTCC |
| | | | | TCCACCCTGACCCTGTCCAAGGCCGACTACGAGAAGCACAAGGTGTAC |
| | | | | GCCTGCGAAGTGACCCACCAGGGCCTGTCTAGCCCCGTGACCAAGTCT |
| | | | | TTCAACCGGGGCGAGTGT |
| 132 | 413D08- | Amino acid sequence | | GFTFRIYG |
| | CDRH1 | of CDRH1 of 413D08 | | |
| | (IMGT) | using IMGT | | |
| 133 | 413D08- | Amino acid sequence | | IWYDGSNK |
| | CDRH2 | of CDRH2 of 413D08 | | |
| | (IMGT) | using IMGT | | |
| 134 | 413D08- | Amino acid sequence | | ARDMDYFGMDV |
| | CDRH3 | of CDRH3 of 413D08 | | |
| | (IMGT) | using IMGT | | |
| 135 | 413D08- | Amino acid sequence | | IYGMH |
| | CDRH1 | of CDRH1 of 413D08 | | |
| | (Kabat) | using Kabat | | |
| 136 | 413D08- | Amino acid sequence | | VIWYDGSNKYYADSVKG |
| | CDRH2 | of CDRH2 of 413D08 | | |
| | (Kabat) | using Kabat | | |
| 137 | 413D08- | Amino acid sequence | | DMDYFGMDV |
| | CDRH3 | of CDRH3 of 413D08 | | |
| | (Kabat) | using Kabat | | |
| 138 | 413D08- | Amino acid sequence | | QVQLVESGGGVVQPGRSLRLSCAASGFTFRIYGMHWVRQAPGKGLEWV |
| | Heavy | of V_{H} of 413D08 | | AVIWYDGSNKYYADSVKGRFTISRDNSDNTLYLQMNSLRAEDTAVYYC |
| | chain | | | ARDMDYFGMDVWGQGTTVTVSS |
| | variable | | | |
| | region | | | |
| 139 | 413D08- | Nucleic acid | | CAGGTGCAGCTGGTGGAGTCTGGGGGAGGCGTGGTCCAGCCTGGGAGG |
| | Heavy | sequence of V_{H} of | | TCCCTGAGACTCTCCTGTGCAGCGTCTGGATTCACCTTCCGTATTTAT |
| | chain | 413D08 | | GGCATGCACTGGGTCCGCCAGGCTCCAGGCAAGGGGCTGGAGTGGGTG |
| | variable | | | GCAGTTATATGGTATGATGGAAGTAATAAATACTATGCTGACTCCGTG |
| | region | | | AAGGGCCGATTCACCATCTCCAGAGACAATTCCGACAACACGCTGTAT |
| | | | | CTGCAAATGAACAGCCTGAGAGCCGAGGACACGGCTGTGTATTACTGT |
| | | | | GCGAGAGATATGGACTACTTCGGTATGGACGTCTGGGGCCAAGGGACC |
| | | | | ACGGTCACCGTCTCCTCAG |
| 140 | 413D08- | Amino acid sequence | | QVQLVESGGGVVQPGRSLRLSCAASGFTFRIYGMHWVRQAPGKGLEWV |
| | full | of 413D08 heavy | | AVIWYDGSNKYYADSVKGRFTISRDNSDNTLYLQMNSLRAEDTAVYYC |
| | heavy | chain | | ARDMDYFGMDVWGQGTTVTVSSASTKGPSVFPLAPSSKSTSGGTAALG |
| | chain | | | CLVKDYFPEPVTVSWNSGALTSGVHTFPAVLQSSGLYSLSSVVTVPSS |
| | sequence | | | SLGTQTYICNVNHKPSNTKVDKKVEPKSCDKTHTCPPCPAPELLGGPS |
| | | | | VFLFPPKPKDTLMISRTPEVTCVVVDVSHEDPEVKFNWYVDGVEVHNA |
| | | | | KTKPREEQYNSTYRVVSVLTVLHQDWLNGKEYKCKVSNKALPAPIEKT |
| | | | | ISKAKGQPREPQVYTLPPSRDELTKNQVSLTCLVKGFYPSDIAVEWES |
| | | | | NGQPENNYKTTPPVLDSDGSFFLYSKLTVDKSRWQQGNVFSCSVMHEA |
| | | | | LHNHYTQKSLSLSPGK |
| 141 | 413D08- | Nucleic acid | | CAGGTGCAGCTGGTGGAGTCTGGGGGAGGCGTGGTCCAGCCTGGGAGG |
| | full | sequence of 413D08 | | TCCCTGAGACTCTCCTGTGCAGCGTCTGGATTCACCTTCCGTATTTAT |
| | heavy | heavy chain | | GGCATGCACTGGGTCCGCCAGGCTCCAGGCAAGGGGCTGGAGTGGGTG |
| | chain | | | GCAGTTATATGGTATGATGGAAGTAATAAATACTATGCTGACTCCGTG |
| | sequence | | | AAGGGCCGATTCACCATCTCCAGAGACAATTCCGACAACACGCTGTAT |
| | | | | CTGCAAATGAACAGCCTGAGAGCCGAGGACACGGCTGTGTATTACTGT |
| | | | | GCGAGAGATATGGACTACTTCGGTATGGACGTCTGGGGCCAAGGGACC |
| | | | | ACGGTCACCGTCTCCTCAGCCAGCACCAAGGGCCCCTCTGTGTTCCCT |
| | | | | CTGGCCCCTTCCAGCAAGTCCACCTCTGGCGGAACAGCCGCTCTGGGC |
| | | | | TGCCTCGTGAAGGACTACTTCCCCGAGCCTGTGACCGTGTCCTGGAAC |
| | | | | TCTGGCGCTCTGACCAGCGGAGTGCACACCTTCCCTGCTGTGCTGCAG |
| | | | | TCCTCCGGCCTGTACTCCCTGTCCTCCGTCGTGACCGTGCCTTCCAGC |
| | | | | TCTCTGGGCACCCAGACCTACATCTGCAACGTGAACCACAAGCCCTCC |
| | | | | AACACCAAGGTGGACAAGAAGGTGGAACCCAAGTCCTGCGACAAGACC |
| | | | | CACACCTGTCCCCCTTGTCCTGCCCCTGAACTGCTGGGCGGACCTTCC |
| | | | | GTGTTCCTGTTCCCCCCAAAGCCCAAGGACACCCTGATGATCTCCCGG |
| | | | | ACCCCCGAAGTGACCTGCGTGGTGGTGGATGTGTCCCACGAGGACCCT |
| | | | | GAAGTGAAGTTCAATTGGTACGTGGACGGCGTGGAAGTGCACAACGCC |
| | | | | AAGACCAAGCCTAGAGAGGAACAGTACAACTCCACCTACCGGGTGGTG |
| | | | | TCCGTGCTGACCGTGCTGCACCAGGATTGGCTGAACGGCAAAGAGTAC |
| | | | | AAGTGCAAGGTGTCCAACAAGGCCCTGCCTGCCCCCATCGAAAAGACC |
| | | | | ATCTCCAAGGCCAAGGGCCAGCCCCGGGAACCCCAGGTGTACACACTG |
| | | | | CCCCCTAGCAGGGACGAGCTGACCAAGAACCAGGTGTCCCTGACCTGT |
| | | | | CTCGTGAAAGGCTTCTACCCCTCCGATATCGCCGTGGAATGGGAGTCC |
| | | | | AACGGCCAGCCTGAGAACAACTACAAGACCACCCCCCCTGTGCTGGAC |
| | | | | TCCGACGGCTCATTCTTCCTGTACAGCAAGCTGACAGTGGACAAGTCC |
| | | | | CGGTGGCAGCAGGGCAACGTGTTCTCCTGCTCCGTGATGCACGAGGCC |
| | | | | CTGCACAACCACTACACCCAGAAGTCCCTGTCCCTGAGCCCCGGCAAG |
| 142 | 413D08- | Amino acid sequence | | QGIRND |
| | CDRL1 | of CDRL1 of 413D08 | | |
| | (IMGT) | using IMGT | | |
| 143 | 413D08- | Amino acid sequence | | AAS |
| | CDRL2 | of CDRL2 of 413D08 | | |
| | (IMGT) | using IMGT | | |
| 144 | 413D08- | Amino acid sequence | | LQHNSYPRT |
| | CDRL3 | of CDRL3 of 413D08 | | |
| | (IMGT) | using IMGT | | |
| 145 | 413D08- | Amino acid sequence | | RASQGIRNDLG |
| | CDRL1 | of CDRL1 of 413D08 | | |
| | (Kabat) | using Kabat | | |
| 146 | 413D08- | Amino acid sequence | | AASSLQS |
| | CDRL2 | of CDRL2 of 413D08 | | |
| | (Kabat) | using Kabat | | |
| 147 | 413D08- | Amino acid sequence | | LQHNSYPRT |
| | CDRL3 | of CDRL3 of 413D08 | | |
| | (Kabat) | using Kabat | | |
| 148 | 413D08- | Amino acid sequence | | DLQMTQSPSSLSASVGDRVTITCRASQGIRNDLGWYQQKPGKAPKRLI |
| | Light | of V_{L} of 413D08 | | YAASSLQSGVPSRFSGSGSGTEFTLTISSLQPEDFATYYCLQHNSYPR |
| | chain | | | TFGQGTKVEIK |
| | variable | | | |
| | region | | | |
| 149 | 413D08- | Nucleic acid | | GACCTCCAGATGACCCAGTCTCCATCCTCCCTGTCTGCATCTGTAGGA |
| | Light | sequence of V_{L} of | | GACAGAGTCACCATCACTTGCCGGGCAAGTCAGGGCATTAGAAATGAT |
| | chain | 413D08 | | TTAGGCTGGTATCAGCAGAAACCAGGGAAAGCCCCTAAGCGCCTGATC |
| | variable | | | TATGCTGCATCCAGTTTGCAAAGTGGGGTCCCATCAAGGTTCAGCGGC |
| | region | | | AGTGGATCTGGGACAGAATTCACTCTCACAATCAGCAGCCTGCAGCCT |
| | | | | GAAGATTTTGCAACTTATTACTGTCTACAGCATAATAGTTACCCTCGG |
| | | | | ACGTTCGGCCAAGGGACCAAGGTGGAAATCAAAC |
| 150 | 413D08- | Amino acid sequence | | DLQMTQSPSSLSASVGDRVTITCRASQGIRNDLGWYQQKPGKAPKRLI |
| | full | of 413D08 light | | YAASSLQSGVPSRFSGSGSGTEFTLTISSLQPEDFATYYCLQHNSYPR |
| | light | chain | | TFGQGTKVEIKRTVAAPSVFIFPPSDEQLKSGTASVVCLLNNFYPREA |
| | chain | | | KVQWKVDNALQSGNSQESVTEQDSKDSTYSLSSTLTLSKADYEKHKVY |
| | sequence | | | ACEVTHQGLSSPVTKSFNRGEC |
| 151 | 413D08- | Nucleic acid | | GACCTCCAGATGACCCAGTCTCCATCCTCCCTGTCTGCATCTGTAGGA |
| | full | sequence of 413D08 | | GACAGAGTCACCATCACTTGCCGGGCAAGTCAGGGCATTAGAAATGAT |
| | light | light chain | | TTAGGCTGGTATCAGCAGAAACCAGGGAAAGCCCCTAAGCGCCTGATC |
| | chain | | | TATGCTGCATCCAGTTTGCAAAGTGGGGTCCCATCAAGGTTCAGCGGC |
| | sequence | | | AGTGGATCTGGGACAGAATTCACTCTCACAATCAGCAGCCTGCAGCCT |
| | | | | GAAGATTTTGCAACTTATTACTGTCTACAGCATAATAGTTACCCTCGG |
| | | | | ACGTTCGGCCAAGGGACCAAGGTGGAAATCAAACGTACGGTGGCCGCT |
| | | | | CCCTCCGTGTTCATCTTCCCACCTTCCGACGAGCAGCTGAAGTCCGGC |
| | | | | ACCGCTTCTGTCGTGTGCCTGCTGAACAACTTCTACCCCCGCGAGGCC |
| | | | | AAGGTGCAGTGGAAGGTGGACAACGCCCTGCAGTCCGGCAACTCCCAG |
| | | | | GAATCCGTGACCGAGCAGGACTCCAAGGACAGCACCTACTCCCTGTCC |
| | | | | TCCACCCTGACCCTGTCCAAGGCCGACTACGAGAAGCACAAGGTGTAC |
| | | | | GCCTGCGAAGTGACCCACCAGGGCCTGTCTAGCCCCGTGACCAAGTCT |
| | | | | TTCAACCGGGGCGAGTGT |
| 152 | 386H03- | Amino acid sequence | | GGSISSSDW |
| | CDRH1 | of CDRH1 of 386H03 | | |
| | (IMGT) | using IMGT | | |
| 153 | 386H03- | Amino acid sequence | | IFHSGNT |
| | CDRH2 | of CDRH2 of 386H03 | | |
| | (IMGT) | using IMGT | | |
| 154 | 386H03- | Amino acid sequence | | VRDGSGSY |
| | CDRH3 | of CDRH3 of 386H03 | | |
| | (IMGT) | using IMGT | | |
| 155 | 386H03- | Amino acid sequence | | SSDWWS |
| | CDRH1 | of CDRH1 of 386H03 | | |
| | (Kabat) | using Kabat | | |
| 156 | 386H03- | Amino acid sequence | | EIFHSGNTNYNPSLKS |
| | CDRH2 | of CDRH2 of 386H03 | | |
| | (Kabat) | using Kabat | | |
| 157 | 386H03- | Amino acid sequence | | DGSGSY |
| | CDRH3 | of CDRH3 of 386H03 | | |
| | (Kabat) | using Kabat | | |
| 158 | 386H03- | Amino acid sequence | | QVQLQESGPGLVKPSGTLSLTCAVSGGSISSSDWWSWVRQPPGKGLEW |
| | Heavy | of V_{H} of 386H03 | | IGEIFHSGNTNYNPSLKSRVTISVDKSKNQISLRLNSVTAADTAVYYC |
| | chain | | | VRDGSGSYWGQGTLVTVSS |
| | variable | | | |
| | region | | | |
| 159 | 386H03- | Nucleic acid | | CAGGTGCAGCTGCAGGAGTCGGGCCCAGGACTGGTGAAGCCTTCGGGG |
| | Heavy | sequence of V_{H} of | | ACCCTGTCCCTCACCTGCGCTGTCTCTGGTGGCTCCATCAGCAGTAGT |
| | chain | 386H03 | | GACTGGTGGAGTTGGGTCCGCCAGCCCCCAGGGAAGGGGCTGGAGTGG |
| | variable | | | ATTGGGGAAATCTTTCATAGTGGGAACACCAACTACAACCCGTCCCTC |
| | region | | | AAGAGTCGAGTCACCATATCAGTAGACAAGTCCAAGAACCAGATCTCC |
| | | | | CTGAGGCTGAACTCTGTGACCGCCGCGGACACGGCCGTGTATTACTGT |
| | | | | GTGAGAGATGGTTCGGGGAGTTACTGGGGCCAGGGAACCCTGGTCACC |
| | | | | GTCTCCTCAG |
| 160 | 386H03- | Amino acid sequence | | QVQLQESGPGLVKPSGTLSLTCAVSGGSISSSDWWSWVRQPPGKGLEW |
| | full | of 386H03 heavy | | IGEIFHSGNTNYNPSLKSRVTISVDKSKNQISLRLNSVTAADTAVYYC |
| | heavy | chain | | VRDGSGSYWGQGTLVTVSSASTKGPSVFPLAPSSKSTSGGTAALGCLV |
| | chain | | | KDYFPEPVTVSWNSGALTSGVHTFPAVLQSSGLYSLSSVVTVPSSSLG |
| | sequence | | | TQTYICNVNHKPSNTKVDKKVEPKSCDKTHTCPPCPAPELLGGPSVEL |
| | | | | FPPKPKDTLMISRTPEVTCVVVDVSHEDPEVKFNWYVDGVEVHNAKTK |
| | | | | PREEQYNSTYRVVSVLTVLHQDWLNGKEYKCKVSNKALPAPIEKTISK |
| | | | | AKGQPREPQVYTLPPSRDELTKNQVSLTCLVKGFYPSDIAVEWESNGQ |
| | | | | PENNYKTTPPVLDSDGSFFLYSKLTVDKSRWQQGNVFSCSVMHEALHN |
| | | | | HYTQKSLSLSPGK |
| 161 | 386H03- | Nucleic acid | | CAGGTGCAGCTGCAGGAGTCGGGCCCAGGACTGGTGAAGCCTTCGGGG |
| | full | sequence of 386H03 | | ACCCTGTCCCTCACCTGCGCTGTCTCTGGTGGCTCCATCAGCAGTAGT |
| | heavy | heavy chain | | GACTGGTGGAGTTGGGTCCGCCAGCCCCCAGGGAAGGGGCTGGAGTGG |
| | chain | | | ATTGGGGAAATCTTTCATAGTGGGAACACCAACTACAACCCGTCCCTC |
| | sequence | | | AAGAGTCGAGTCACCATATCAGTAGACAAGTCCAAGAACCAGATCTCC |
| | | | | CTGAGGCTGAACTCTGTGACCGCCGCGGACACGGCCGTGTATTACTGT |
| | | | | GTGAGAGATGGTTCGGGGAGTTACTGGGGCCAGGGAACCCTGGTCACC |
| | | | | GTCTCCTCAGCCAGCACCAAGGGCCCCTCTGTGTTCCCTCTGGCCCCT |
| | | | | TCCAGCAAGTCCACCTCTGGCGGAACAGCCGCTCTGGGCTGCCTCGTG |
| | | | | AAGGACTACTTCCCCGAGCCTGTGACCGTGTCCTGGAACTCTGGCGCT |
| | | | | CTGACCAGCGGAGTGCACACCTTCCCTGCTGTGCTGCAGTCCTCCGGC |
| | | | | CTGTACTCCCTGTCCTCCGTCGTGACCGTGCCTTCCAGCTCTCTGGGC |
| | | | | ACCCAGACCTACATCTGCAACGTGAACCACAAGCCCTCCAACACCAAG |
| | | | | GTGGACAAGAAGGTGGAACCCAAGTCCTGCGACAAGACCCACACCTGT |
| | | | | CCCCCTTGTCCTGCCCCTGAACTGCTGGGCGGACCTTCCGTGTTCCTG |
| | | | | TTCCCCCCAAAGCCCAAGGACACCCTGATGATCTCCCGGACCCCCGAA |
| | | | | GTGACCTGCGTGGTGGTGGATGTGTCCCACGAGGACCCTGAAGTGAAG |
| | | | | TTCAATTGGTACGTGGACGGCGTGGAAGTGCACAACGCCAAGACCAAG |
| | | | | CCTAGAGAGGAACAGTACAACTCCACCTACCGGGTGGTGTCCGTGCTG |
| | | | | ACCGTGCTGCACCAGGATTGGCTGAACGGCAAAGAGTACAAGTGCAAG |
| | | | | GTGTCCAACAAGGCCCTGCCTGCCCCCATCGAAAAGACCATCTCCAAG |
| | | | | GCCAAGGGCCAGCCCCGGGAACCCCAGGTGTACACACTGCCCCCTAGC |
| | | | | AGGGACGAGCTGACCAAGAACCAGGTGTCCCTGACCTGTCTCGTGAAA |
| | | | | GGCTTCTACCCCTCCGATATCGCCGTGGAATGGGAGTCCAACGGCCAG |
| | | | | CCTGAGAACAACTACAAGACCACCCCCCCTGTGCTGGACTCCGACGGC |
| | | | | TCATTCTTCCTGTACAGCAAGCTGACAGTGGACAAGTCCCGGTGGCAG |
| | | | | CAGGGCAACGTGTTCTCCTGCTCCGTGATGCACGAGGCCCTGCACAAC |
| | | | | CACTACACCCAGAAGTCCCTGTCCCTGAGCCCCGGCAAG |
| 162 | 386H03- | Amino acid sequence | | QSVLYSSNNKNY |
| | CDRL1 | of CDRL1 of 386H03 | | |
| | (IMGT) | using IMGT | | |
| 163 | 386H03- | Amino acid sequence | | WAS |
| | CDRL2 | of CDRL2 of 386H03 | | |
| | (IMGT) | using IMGT | | |
| 164 | 386H03- | Amino acid sequence | | QQYYSTRS |
| | CDRL3 | of CDRL3 of 386H03 | | |
| | (IMGT) | using IMGT | | |
| 165 | 386H03- | Amino acid sequence | | KSSQSVLYSSNNKNYLA |
| | CDRL1 | of CDRL1 of 386H03 | | |
| | (Kabat) | using Kabat | | |
| 166 | 386H03- | Amino acid sequence | | WASTRES |
| | CDRL2 | of CDRL2 of 386H03 | | |
| | (Kabat) | using Kabat | | |
| 167 | 386H03- | Amino acid sequence | | QQYYSTRS |
| | CDRL3 | of CDRL3 of 386H03 | | |
| | (Kabat) | using Kabat | | |
| 168 | 386H03- | Amino acid sequence | | DIVMTQSPDSLAVSLGERATINCKSSQSVLYSSNNKNYLAWYQQKPGQ |
| | Light | of V_{L} of 386H03 | | PPKLLIYWASTRESGVPDRFSGSGSGTDFTLTISSLQAEDVAVYYCQQ |
| | chain | | | YYSTRSFGQGTKLEIK |
| | variable | | | |
| | region | | | |
| 169 | 386H03- | Nucleic acid | | GACATCGTGATGACCCAGTCTCCAGACTCCCTGGCTGTGTCTCTGGGC |
| | Light | sequence of V_{L} of | | GAGAGGGCCACCATCAACTGCAAGTCCAGCCAGAGTGTTTTATACAGC |
| | chain | 386H03 | | TCCAACAATAAGAACTACTTAGCTTGGTACCAGCAGAAACCAGGACAG |
| | variable | | | CCTCCTAAACTGCTCATTTACTGGGCATCTACCCGGGAATCCGGGGTC |
| | region | | | CCTGACCGATTCAGTGGCAGCGGGTCTGGGACAGATTTCACTCTCACC |
| | | | | ATCAGCAGCCTGCAGGCTGAAGATGTGGCAGTTTATTACTGTCAGCAA |
| | | | | TATTATAGTACTCGCAGTTTTGGCCAGGGGACCAAGCTGGAGATCAAA |
| | | | | C |
| 170 | 386H03- | Amino acid sequence | | DIVMTQSPDSLAVSLGERATINCKSSQSVLYSSNNKNYLAWYQQKPGQ |
| | full | of 386H03 light | | PPKLLIYWASTRESGVPDRFSGSGSGTDFTLTISSLQAEDVAVYYCQQ |
| | light | chain | | YYSTRSFGQGTKLEIKRTVAAPSVFIFPPSDEQLKSGTASVVCLLNNF |
| | chain | | | YPREAKVQWKVDNALQSGNSQESVTEQDSKDSTYSLSSTLTLSKADYE |
| | sequence | | | KHKVYACEVTHQGLSSPVTKSFNRGEC |
| 171 | 386H03- | Nucleic acid | | GACATCGTGATGACCCAGTCTCCAGACTCCCTGGCTGTGTCTCTGGGC |
| | full | sequence of 386H03 | | GAGAGGGCCACCATCAACTGCAAGTCCAGCCAGAGTGTTTTATACAGC |
| | light | light chain | | TCCAACAATAAGAACTACTTAGCTTGGTACCAGCAGAAACCAGGACAG |
| | chain | | | CCTCCTAAACTGCTCATTTACTGGGCATCTACCCGGGAATCCGGGGTC |
| | sequence | | | CCTGACCGATTCAGTGGCAGCGGGTCTGGGACAGATTTCACTCTCACC |
| | | | | ATCAGCAGCCTGCAGGCTGAAGATGTGGCAGTTTATTACTGTCAGCAA |
| | | | | TATTATAGTACTCGCAGTTTTGGCCAGGGGACCAAGCTGGAGATCAAA |
| | | | | CGTACGGTGGCCGCTCCCTCCGTGTTCATCTTCCCACCTTCCGACGAG |
| | | | | CAGCTGAAGTCCGGCACCGCTTCTGTCGTGTGCCTGCTGAACAACTTC |
| | | | | TACCCCCGCGAGGCCAAGGTGCAGTGGAAGGTGGACAACGCCCTGCAG |
| | | | | TCCGGCAACTCCCAGGAATCCGTGACCGAGCAGGACTCCAAGGACAGC |
| | | | | ACCTACTCCCTGTCCTCCACCCTGACCCTGTCCAAGGCCGACTACGAG |
| | | | | AAGCACAAGGTGTACGCCTGCGAAGTGACCCACCAGGGCCTGTCTAGC |
| | | | | CCCGTGACCAAGTCTTTCAACCGGGGCGAGTGT |
| 172 | 389A03- | Amino acid sequence | | GGSISSSSYY |
| | CDRH1 | of CDRH1 of 389A03 | | |
| | (IMGT) | using IMGT | | |
| 173 | 389A03- | Amino acid sequence | | IYSTGYT |
| | CDRH2 | of CDRH2 of 389A03 | | |
| | (IMGT) | using IMGT | | |
| 174 | 389A03- | Amino acid sequence | | AISTAAGPEYFHR |
| | CDRH3 | of CDRH3 of 389A03 | | |
| | (IMGT) | using IMGT | | |
| 175 | 389A03- | Amino acid sequence | | SSSYYCG |
| | CDRH1 | of CDRH1 of 389A03 | | |
| | (Kabat) | using Kabat | | |
| 176 | 389A03- | Amino acid sequence | | SIYSTGYTYYNPSLKS |
| | CDRH2 | of CDRH2 of 389A03 | | |
| | (Kabat) | using Kabat | | |
| 177 | 389A03- | Amino acid sequence | | STAAGPEYFHR |
| | CDRH3 | of CDRH3 of 389A03 | | |
| | (Kabat) | using Kabat | | |
| 178 | 389A03- | Amino acid sequence | | QLQESGPGLVKPSETLSLTCTVSGGSISSSSYYCGWIRQPPGKGLDWI |
| | Heavy | of V_{H} of 389A03 | | GSIYSTGYTYYNPSLKSRVTISIDTSKNQFSCLILTSVTAADTAVYYC |
| | chain | | | AISTAAGPEYFHRWGQGTLVTVSS |
| | variable | | | |
| | region | | | |
| 179 | 389A03- | Nucleic acid | | CAGCTGCAGGAGTCGGGCCCAGGCCTGGTGAAGCCTTCGGAGACCCTG |
| | Heavy | sequence of V_{H} of | | TCCCTCACCTGCACTGTCTCTGGTGGCTCCATCAGCAGTAGTAGTTAT |
| | chain | 389A03 | | TACTGCGGCTGGATCCGCCAGCCCCCTGGGAAGGGGCTGGACTGGATT |
| | variable | | | GGGAGTATCTATTCTACTGGGTACACCTACTACAACCCGTCCCTCAAG |
| | region | | | AGTCGAGTCACCATTTCCATAGACACGTCCAAGAACCAGTTCTCATGC |
| | | | | CTGATACTGACCTCTGTGACCGCCGCAGACACGGCTGTGTATTACTGT |
| | | | | GCGATAAGTACAGCAGCTGGCCCTGAATACTTCCATCGCTGGGGCCAG |
| | | | | GGCACCCTGGTCACCGTCTCCTCAG |
| 180 | 389A03- | Amino acid sequence | | QLQESGPGLVKPSETLSLTCTVSGGSISSSSYYCGWIRQPPGKGLDWI |
| | full | of 389A03 heavy | | GSIYSTGYTYYNPSLKSRVTISIDTSKNQFSCLILTSVTAADTAVYYC |
| | heavy | chain | | AISTAAGPEYFHRWGQGTLVTVSSASTKGPSVFPLAPSSKSTSGGTAA |
| | chain | | | LGCLVKDYFPEPVTVSWNSGALTSGVHTFPAVLQSSGLYSLSSVVTVP |
| | sequence | | | SSSLGTQTYICNVNHKPSNTKVDKKVEPKSCDKTHTCPPCPAPELLGG |
| | | | | PSVFLFPPKPKDTLMISRTPEVTCVVVDVSHEDPEVKFNWYVDGVEVH |
| | | | | NAKTKPREEQYNSTYRVVSVLTVLHQDWLNGKEYKCKVSNKALPAPIE |
| | | | | KTISKAKGQPREPQVYTLPPSRDELTKNQVSLTCLVKGFYPSDIAVEW |
| | | | | ESNGQPENNYKTTPPVLDSDGSFFLYSKLTVDKSRWQQGNVFSCSVMH |
| | | | | EALHNHYTQKSLSLSPGK |
| 181 | 389A03- | Nucleic acid | | CAGCTGCAGGAGTCGGGCCCAGGCCTGGTGAAGCCTTCGGAGACCCTG |
| | full | sequence of 389A03 | | TCCCTCACCTGCACTGTCTCTGGTGGCTCCATCAGCAGTAGTAGTTAT |
| | heavy | heavy chain | | TACTGCGGCTGGATCCGCCAGCCCCCTGGGAAGGGGCTGGACTGGATT |
| | chain | | | GGGAGTATCTATTCTACTGGGTACACCTACTACAACCCGTCCCTCAAG |
| | sequence | | | AGTCGAGTCACCATTTCCATAGACACGTCCAAGAACCAGTTCTCATGC |
| | | | | CTGATACTGACCTCTGTGACCGCCGCAGACACGGCTGTGTATTACTGT |
| | | | | GCGATAAGTACAGCAGCTGGCCCTGAATACTTCCATCGCTGGGGCCAG |
| | | | | GGCACCCTGGTCACCGTCTCCTCAGCCAGCACCAAGGGCCCCTCTGTG |
| | | | | TTCCCTCTGGCCCCTTCCAGCAAGTCCACCTCTGGCGGAACAGCCGCT |
| | | | | CTGGGCTGCCTCGTGAAGGACTACTTCCCCGAGCCTGTGACCGTGTCC |
| | | | | TGGAACTCTGGCGCTCTGACCAGCGGAGTGCACACCTTCCCTGCTGTG |
| | | | | CTGCAGTCCTCCGGCCTGTACTCCCTGTCCTCCGTCGTGACCGTGCCT |
| | | | | TCCAGCTCTCTGGGCACCCAGACCTACATCTGCAACGTGAACCACAAG |
| | | | | CCCTCCAACACCAAGGTGGACAAGAAGGTGGAACCCAAGTCCTGCGAC |
| | | | | AAGACCCACACCTGTCCCCCTTGTCCTGCCCCTGAACTGCTGGGCGGA |
| | | | | CCTTCCGTGTTCCTGTTCCCCCCAAAGCCCAAGGACACCCTGATGATC |
| | | | | TCCCGGACCCCCGAAGTGACCTGCGTGGTGGTGGATGTGTCCCACGAG |
| | | | | GACCCTGAAGTGAAGTTCAATTGGTACGTGGACGGCGTGGAAGTGCAC |
| | | | | AACGCCAAGACCAAGCCTAGAGAGGAACAGTACAACTCCACCTACCGG |
| | | | | GTGGTGTCCGTGCTGACCGTGCTGCACCAGGATTGGCTGAACGGCAAA |
| | | | | GAGTACAAGTGCAAGGTGTCCAACAAGGCCCTGCCTGCCCCCATCGAA |
| | | | | AAGACCATCTCCAAGGCCAAGGGCCAGCCCCGGGAACCCCAGGTGTAC |
| | | | | ACACTGCCCCCTAGCAGGGACGAGCTGACCAAGAACCAGGTGTCCCTG |
| | | | | ACCTGTCTCGTGAAAGGCTTCTACCCCTCCGATATCGCCGTGGAATGG |
| | | | | GAGTCCAACGGCCAGCCTGAGAACAACTACAAGACCACCCCCCCTGTG |
| | | | | CTGGACTCCGACGGCTCATTCTTCCTGTACAGCAAGCTGACAGTGGAC |
| | | | | AAGTCCCGGTGGCAGCAGGGCAACGTGTTCTCCTGCTCCGTGATGCAC |
| | | | | GAGGCCCTGCACAACCACTACACCCAGAAGTCCCTGTCCCTGAGCCCC |
| | | | | GGCAAG |
| 182 | 389A03- | Amino acid sequence | | QSVLYSSNSKNF |
| | CDRL1 | of CDRL1 of 389A03 | | |
| | (IMGT) | using IMGT | | |
| 183 | 389A03- | Amino acid sequence | | WAS |
| | CDRL2 | of CDRL2 of 389A03 | | |
| | (IMGT) | using IMGT | | |
| 184 | 389A03- | Amino acid sequence | | QQYYSTPRT |
| | CDRL3 | of CDRL3 of 389A03 | | |
| | (IMGT) | using IMGT | | |
| 185 | 389A03- | Amino acid sequence | | KSSQSVLYSSNSKNFLA |
| | CDRL1 | of CDRL1 of 389A03 | | |
| | (Kabat) | using Kabat | | |
| 186 | 389A03- | Amino acid sequence | | WASTRGS |
| | CDRL2 | of CDRL2 of 389A03 | | |
| | (Kabat) | using Kabat | | |
| 187 | 389A03- | Amino acid sequence | | QQYYSTPRT |
| | CDRL3 | of CDRL3 of 389A03 | | |
| | (Kabat) | using Kabat | | |
| 188 | 389A03- | Amino acid sequence | | DIVMTQSPDSLAVSLGERATINCKSSQSVLYSSNSKNFLAWYQQKPGQ |
| | Light | of V_{L} of 389A03 | | PPKLFIYWASTRGSGVPDRISGSGSGTDENLTISSLQAEDVAVYYCQQ |
| | chain | | | YYSTPRTFGQGTKVEIK |
| | variable | | | |
| | region | | | |
| 189 | 389A03- | Nucleic acid | | GACATCGTGATGACCCAGTCTCCAGACTCCCTGGCTGTGTCTCTGGGC |
| | Light | sequence of V_{L} of | | GAGAGGGCCACCATCAACTGCAAGTCCAGCCAGAGTGTTTTATACAGC |
| | chain | 389A03 | | TCCAACAGTAAGAACTTCTTAGCTTGGTACCAGCAGAAACCGGGACAG |
| | variable | | | CCTCCTAAGCTGTTCATTTACTGGGCATCTACCCGGGGATCCGGGGTC |
| | region | | | CCTGACCGAATCAGTGGCAGCGGGTCTGGGACAGATTTCAATCTCACC |
| | | | | ATCAGCAGCCTGCAGGCTGAAGATGTGGCAGTTTATTACTGTCAACAA |
| | | | | TATTATAGTACTCCTCGGACGTTCGGCCAAGGGACCAAGGTGGAGATC |
| | | | | AAAC |
| 190 | 389A03- | Amino acid sequence | | DIVMTQSPDSLAVSLGERATINCKSSQSVLYSSNSKNFLAWYQQKPGQ |
| | full | of 389A03 light | | PPKLFIYWASTRGSGVPDRISGSGSGTDENLTISSLQAEDVAVYYCQQ |
| | light | chain | | YYSTPRTFGQGTKVEIKRTVAAPSVFIFPPSDEQLKSGTASVVCLLNN |
| | chain | | | FYPREAKVQWKVDNALQSGNSQESVTEQDSKDSTYSLSSTLTLSKADY |
| | sequence | | | EKHKVYACEVTHQGLSSPVTKSFNRGEC |
| 191 | 389A03- | Nucleic acid | | GACATCGTGATGACCCAGTCTCCAGACTCCCTGGCTGTGTCTCTGGGC |
| | full | sequence of 389A03 | | GAGAGGGCCACCATCAACTGCAAGTCCAGCCAGAGTGTTTTATACAGC |
| | light | light chain | | TCCAACAGTAAGAACTTCTTAGCTTGGTACCAGCAGAAACCGGGACAG |
| | chain | | | CCTCCTAAGCTGTTCATTTACTGGGCATCTACCCGGGGATCCGGGGTC |
| | sequence | | | CCTGACCGAATCAGTGGCAGCGGGTCTGGGACAGATTTCAATCTCACC |
| | | | | ATCAGCAGCCTGCAGGCTGAAGATGTGGCAGTTTATTACTGTCAACAA |
| | | | | TATTATAGTACTCCTCGGACGTTCGGCCAAGGGACCAAGGTGGAGATC |
| | | | | AAACGTACGGTGGCCGCTCCCTCCGTGTTCATCTTCCCACCTTCCGAC |
| | | | | GAGCAGCTGAAGTCCGGCACCGCTTCTGTCGTGTGCCTGCTGAACAAC |
| | | | | TTCTACCCCCGCGAGGCCAAGGTGCAGTGGAAGGTGGACAACGCCCTG |
| | | | | CAGTCCGGCAACTCCCAGGAATCCGTGACCGAGCAGGACTCCAAGGAC |
| | | | | AGCACCTACTCCCTGTCCTCCACCCTGACCCTGTCCAAGGCCGACTAC |
| | | | | GAGAAGCACAAGGTGTACGCCTGCGAAGTGACCCACCAGGGCCTGTCT |
| | | | | AGCCCCGTGACCAAGTCTTTCAACCGGGGCGAGTGT |
| 192 | Human | IGHG*01 | Heavy Chain | gcttccaccaagggcccatccgtcttccccctggcgccctgctccagg |
| | IgG4 | & | Constant | agcacctccgagagcacagccgccctgggctgcctggtcaaggactac |
| | heavy | IGHG4*04 | Region | ttccccgaaccggtgacggtgtcgtggaactcaggcgccctgaccagc |
| | chain | | Nucleotide | ggcgtgcacaccttcccggctgtcctacagtcctcaggactctactcc |
| | constant | | Sequence | ctcagcagcgtggtgaccgtgccctccagcagcttgggcacgaagacc |
| | region | | | tacacctgcaacgtagatcacaagcccagcaacaccaaggtggacaag |
| | #1 | | | agagttgagtccaaatatggtcccccatgcccatcatgcccagcacct |
| | | | | gagttcctggggggaccatcagtcttcctgttccccccaaaacccaag |
| | | | | gacactctcatgatctcccggacccctgaggtcacgtgcgtggtggtg |
| | | | | gacgtgagccaggaagaccccgaggtccagttcaactggtacgtggat |
| | | | | ggcgtggaggtgcataatgccaagacaaagccgcgggaggagcagttc |
| | | | | aacagcacgtaccgtgtggtcagcgtcctcaccgtcctgcaccaggac |
| | | | | tggctgaacggcaaggagtacaagtgcaaggtctccaacaaaggcctc |
| | | | | ccgtcctccatcgagaaaaccatctccaaagccaaagggcagccccga |
| | | | | gagccacaggtgtacaccctgcccccatcccaggaggagatgaccaag |
| | | | | aaccaggtcagcctgacctgcctggtcaaaggcttctaccccagcgac |
| | | | | atcgccgtggagtgggagagcaatgggcagccggagaacaactacaag |
| | | | | accacgcctcccgtgctggactccgacggctccttcttcctctacagc |
| | | | | aggctaaccgtggacaagagcaggtggcaggaggggaatgtcttctca |
| | | | | tgctccgtgatgcatgaggctctgcacaaccactacacacagaagagc |
| | | | | ctctccctgtctctgggtaaa |
| 193 | | | Heavy Chain | ASTKGPSVFPLAPCSRSTSESTAALGCLVKDYFPEPVTVSWNSGALTS |
| | | | Constant | GVHTFPAVLQSSGLYSLSSVVTVPSSSLGTKTYTCNVDHKPSNTKVDK |
| | | | Region | RVESKYGPPCPSCPAPEFLGGPSVFLFPPKPKDTLMISRTPEVTCVVV |
| | | | Amino Acid | DVSQEDPEVQFNWYVDGVEVHNAKTKPREEQFNSTYRVVSVLTVLHQD |
| | | | Sequence | WLNGKEYKCKVSNKGLPSSIEKTISKAKGQPREPQVYTLPPSQEEMTK |
| | | | | NQVSLTCLVKGFYPSDIAVEWESNGQPENNYKTTPPVLDSDGSFFLYS |
| | | | | RLTVDKSRWQEGNVFSCSVMHEALHNHYTQKSLSLSLGK |
| 194 | Human | IGHG*02 | Heavy Chain | gcttccaccaagggcccatccgtcttccccctggcgccctgctccagg |
| | IgG4 | | Constant | agcacctccgagagcacagccgccctgggctgcctggtcaaggactac |
| | heavy | | Region | ttccccgaaccggtgacggtgtcgtggaactcaggcgccctgaccagc |
| | chain | | Nucleotide | ggcgtgcacaccttcccggctgtcctacagtcctcaggactctactcc |
| | constant | | Sequence | ctcagcagcgtggtgaccgtgccctccagcagcttgggcacgaagacc |
| | region | | | tacacctgcaacgtagatcacaagcccagcaacaccaaggtggacaag |
| | #2 | | | agagttgagtccaaatatggtcccccgtgcccatcatgcccagcacct |
| | | | | gagttcctggggggaccatcagtcttcctgttccccccaaaacccaag |
| | | | | gacactctcatgatctcccggacccctgaggtcacgtgcgtggtggtg |
| | | | | gacgtgagccaggaagaccccgaggtccagttcaactggtacgtggat |
| | | | | ggcgtggaggtgcataatgccaagacaaagccgcgggaggagcagttc |
| | | | | aacagcacgtaccgtgtggtcagcgtcctcaccgtcgtgcaccaggac |
| | | | | tggctgaacggcaaggagtacaagtgcaaggtctccaacaaaggcctc |
| | | | | ccgtcctccatcgagaaaaccatctccaaagccaaagggcagccccga |
| | | | | gagccacaggtgtacaccctgcccccatcccaggaggagatgaccaag |
| | | | | aaccaggtcagcctgacctgcctggtcaaaggcttctaccccagcgac |
| | | | | atcgccgtggagtgggagagcaatgggcagccggagaacaactacaag |
| | | | | accacgcctcccgtgctggactccgacggctccttcttcctctacagc |
| | | | | aggctaaccgtggacaagagcaggtggcaggaggggaatgtcttctca |
| | | | | tgctccgtgatgcatgaggctctgcacaaccactacacgcagaagagc |
| | | | | ctctccctgtctctgggtaaa |
| 195 | | | Heavy Chain | ASTKGPSVFPLAPCSRSTSESTAALGCLVKDYFPEPVTVSWNSGALTS |
| | | | Constant | GVHTFPAVLQSSGLYSLSSVVTVPSSSLGTKTYTCNVDHKPSNTKVDK |
| | | | Region | RVESKYGPPCPSCPAPEFLGGPSVFLFPPKPKDTLMISRTPEVTCVVV |
| | | | Amino Acid | DVSQEDPEVQFNWYVDGVEVHNAKTKPREEQFNSTYRVVSVLTVVHQD |
| | | | Sequence | WLNGKEYKCKVSNKGLPSSIEKTISKAKGQPREPQVYTLPPSQEEMTK |
| | | | | NQVSLTCLVKGFYPSDIAVEWESNGQPENNYKTTPPVLDSDGSFFLYS |
| | | | | RLTVDKSRWQEGNVFSCSVMHEALHNHYTQKSLSLSLGK |
| 196 | Human | IGHG*03 | Heavy Chain | gcttccaccaagggcccatccgtcttccccctggcgccctgctccagg |
| | IgG4 | | Constant | agcacctccgagagcacagccgccctgggctgcctggtcaaggactac |
| | heavy | | Region | ttccccgaaccggtgacggtgtcgtggaactcaggcgccctgaccagc |
| | chain | | Nucleotide | ggcgtgcacaccttcccggctgtcctacagtcctcaggactctactcc |
| | constant | | Sequence | ctcagcagcgtggtgaccgtgccctccagcagcttgggcacgaagacc |
| | region | | | tacacctgcaacgtagatcacaagcccagcaacaccaaggtggacaag |
| | #3 | | | agagttgagtccaaatatggtcccccatgcccatcatgcccagcacct |
| | | | | gagttcctggggggaccatcagtcttcctgttccccccaaaacccaag |
| | | | | gacactctcatgatctcccggacccctgaggtcacgtgcgtggtggtg |
| | | | | gacgtgagccaggaagaccccgaggtccagttcaactggtacgtggat |
| | | | | ggcgtggaggtgcataatgccaagacaaagccgcgggaggagcagttc |
| | | | | aacagcacgtaccgtgtggtcagcgtcctcaccgtcctgcaccaggac |
| | | | | tggctgaacggcaaggagtacaagtgcaaggtctccaacaaaggcctc |
| | | | | ccgtcctccatcgagaaaaccatctccaaagccaaagggcagccccga |
| | | | | gagccacaggtgtacaccctgcccccatcccaggaggagatgaccaag |
| | | | | aaccaggtcagcctgacctgcctggtcaaaggcttctaccccagcgac |
| | | | | atcgccgtggagtgggagagcaatgggcagccggagaacaactacaag |
| | | | | accacgcctcccgtgctggactccgacggctccttcttcctctacagc |
| | | | | aagctcaccgtggacaagagcaggtggcaggaggggaacgtcttctca |
| | | | | tgctccgtgatgcatgaggctctgcacaaccactacacgcagaagagc |
| | | | | ctctccctgtctctgggtaaa |
| 197 | | | Heavy Chain | ASTKGPSVFPLAPCSRSTSESTAALGCLVKDYFPEPVTVSWNSGALTS |
| | | | Constant | GVHTFPAVLQSSGLYSLSSVVTVPSSSLGTKTYTCNVDHKPSNTKVDK |
| | | | Region | RVESKYGPPCPSCPAPEFLGGPSVFLFPPKPKDTLMISRTPEVTCVVV |
| | | | Amino Acid | DVSQEDPEVQFNWYVDGVEVHNAKTKPREEQFNSTYRVVSVLTVLHQD |
| | | | Sequence | WLNGKEYKCKVSNKGLPSSIEKTISKAKGQPREPQVYTLPPSQEEMTK |
| | | | | NQVSLTCLVKGFYPSDIAVEWESNGQPENNYKTTPPVLDSDGSFFLYS |
| | | | | KLTVDKSRWQEGNVFSCSVMHEALHNHYTQKSLSLSLGK |
| 198 | IgG4 | -IgG4- | Heavy Chain | gcctccaccaagggcccatccgtcttccccctggcgccctgctccagg |
| | heavy | PE | Constant | agcacctccgagagcacggccgccctgggctgcctggtcaaggactac |
| | chain | | Region | ttccccgaaccagtgacggtgtcgtggaactcaggcgccctgaccagc |
| | constant | | Nucleotide | ggcgtgcacaccttcccggctgtcctacagtcctcaggactctactcc |
| | region- | | Sequence- | ctcagcagcgtggtgaccgtgccctccagcagcttgggcacgaagacc |
| | IgG4-PE | | Synthetic | tacacctgcaacgtagatcacaagcccagcaacaccaaggtggacaag |
| | | | Version A | agagttgagtccaaatatggtcccccatgcccaccatgcccagcgcct |
| | | | | gaatttgaggggggaccatcagtcttcctgttccccccaaaacccaag |
| | | | | gacactctcatgatctcccggacccctgaggtcacgtgcgtggtggtg |
| | | | | gacgtgagccaggaagaccccgaggtccagttcaactggtacgtggat |
| | | | | ggcgtggaggtgcataatgccaagacaaagccgcgggaggagcagttc |
| | | | | aacagcacgtaccgtgtggtcagcgtcctcaccgtcctgcaccaggac |
| | | | | tggctgaacggcaaggagtacaagtgcaaggtctccaacaaaggcctc |
| | | | | ccgtcatcgatcgagaaaaccatctccaaagccaaagggcagccccga |
| | | | | gagccacaggtgtacaccctgcccccatcccaggaggagatgaccaag |
| | | | | aaccaggtcagcctgacctgcctggtcaaaggcttctaccccagcgac |
| | | | | atcgccgtggagtgggagagcaatgggcagccggagaacaactacaag |
| | | | | accacgcctcccgtgctggactccgacggatccttcttcctctacagc |
| | | | | aggctaaccgtggacaagagcaggtggcaggaggggaatgtcttctca |
| | | | | tgctccgtgatgcatgaggctctgcacaaccactacacacagaagagc |
| | | | | ctctccctgtctctgggtaaa |
| 199 | IgG4 | | Heavy Chain | ASTKGPSVFPLAPCSRSTSESTAALGCLVKDYFPEPVTVSWNSGALTS |
| | heavy | | Constant | GVHTFPAVLQSSGLYSLSSVVTVPSSSLGTKTYTCNVDHKPSNTKVDK |
| | chain | | Region | RVESKYGPPCPPCPAPEFEGGPSVFLFPPKPKDTLMISRTPEVTCVVV |
| | constant | | Amino Acid | DVSQEDPEVQFNWYVDGVEVHNAKTKPREEQFNSTYRVVSVLTVLHQD |
| | region- | | Sequence | WLNGKEYKCKVSNKGLPSSIEKTISKAKGQPREPQVYTLPPSQEEMTK |
| | IgG4-PE | | Encoded by | NQVSLTCLVKGFYPSDIAVEWESNGQPENNYKTTPPVLDSDGSFFLYS |
| | | | Synthetic | RLTVDKSRWQEGNVFSCSVMHEALHNHYTQKSLSLSLGK |
| | | | Version A, | |
| | | | B & C (Two | |
| | | | residues | |
| | | | that differ | |
| | | | from the | |
| | | | wild-type | |
| | | | sequence | |
| | | | are | |
| | | | identified | |
| | | | in bold) | |
| 200 | IgG4 | | Heavy Chain | Gcctccaccaagggacctagcgtgttccctctcgccccctgttccagg |
| | heavy | | Constant | tccacaagcgagtccaccgctgccctcggctgtctggtgaaagactac |
| | chain | | Region | tttcccgagcccgtgaccgtctcctggaatagcggagccctgacctcc |
| | constant | | Nucleotide | ggcgtgcacacatttcccgccgtgctgcagagcagcggactgtatagc |
| | region- | | Sequence- | ctgagcagcgtggtgaccgtgcccagctccagcctcggcaccaaaacc |
| | IgG4-PE | | Synthetic | tacacctgcaacgtggaccacaagccctccaacaccaaggtggacaag |
| | | | Version B | cgggtggagagcaagtacggccccccttgccctccttgtcctgcccct |
| | | | | gagttcgagggaggaccctccgtgttcctgtttccccccaaacccaag |
| | | | | gacaccctgatgatctcccggacacccgaggtgacctgtgtggtcgtg |
| | | | | gacgtcagccaggaggaccccgaggtgcagttcaactggtatgtggac |
| | | | | ggcgtggaggtgcacaatgccaaaaccaagcccagggaggagcagttc |
| | | | | aattccacctacagggtggtgagcgtgctgaccgtcctgcatcaggat |
| | | | | tggctgaacggcaaggagtacaagtgcaaggtgtccaacaagggactg |
| | | | | cccagctccatcgagaagaccatcagcaaggctaagggccagccgagg |
| | | | | gagccccaggtgtataccctgcctcctagccaggaagagatgaccaag |
| | | | | aaccaagtgtccctgacctgcctggtgaagggattctacccctccgac |
| | | | | atcgccgtggagtgggagagcaatggccagcccgagaacaactacaaa |
| | | | | acaacccctcccgtgctcgatagcgacggcagcttctttctctacagc |
| | | | | cggctgacagtggacaagagcaggtggcaggagggcaacgtgttctcc |
| | | | | tgttccgtgatgcacgaggccctgcacaatcactacacccagaagagc |
| | | | | ctctccctgtccctgggcaag |
| 201 | IgG4 | | Heavy Chain | gccagcaccaagggcccttccgtgttccccctggccccttgcagcagg |
| | heavy | | Constant | agcacctccgaatccacagctgccctgggctgtctggtgaaggactac |
| | chain | | Region | tttcccgagcccgtgaccgtgagctggaacagcggcgctctgacatcc |
| | constant | | Nucleotide | ggcgtccacacctttcctgccgtcctgcagtcctccggcctctactcc |
| | region- | | Sequence- | ctgtcctccgtggtgaccgtgcctagctcctccctcggcaccaagacc |
| | IgG4-PE | | Synthetic | tacacctgtaacgtggaccacaaaccctccaacaccaaggtggacaaa |
| | | | Version C | cgggtcgagagcaagtacggccctccctgccctccttgtcctgccccc |
| | | | | gagttcgaaggcggacccagcgtgttcctgttccctcctaagcccaag |
| | | | | gacaccctcatgatcagccggacacccgaggtgacctgcgtggtggtg |
| | | | | gatgtgagccaggaggaccctgaggtccagttcaactggtatgtggat |
| | | | | ggcgtggaggtgcacaacgccaagacaaagccccgggaagagcagttc |
| | | | | aactccacctacagggtggtcagcgtgctgaccgtgctgcatcaggac |
| | | | | tggctgaacggcaaggagtacaagtgcaaggtcagcaataagggactg |
| | | | | cccagcagcatcgagaagaccatctccaaggctaaaggccagccccgg |
| | | | | gaacctcaggtgtacaccctgcctcccagccaggaggagatgaccaag |
| | | | | aaccaggtgagcctgacctgcctggtgaagggattctacccttccgac |
| | | | | atcgccgtggagtgggagtccaacggccagcccgagaacaattataag |
| | | | | accacccctcccgtcctcgacagcgacggatccttctttctgtactcc |
| | | | | aggctgaccgtggataagtccaggtggcaggaaggcaacgtgttcagc |
| | | | | tgctccgtgatgcacgaggccctgcacaatcactacacccagaagtcc |
| | | | | ctgagcctgtccctgggaaag |
| 202 | IgG4 | | Heavy Chain | gcctccaccaagggcccatccgtcttccccctggcgccctgctccagg |
| | heavy | | Constant | agcacctccgagagcacggccgccctgggctgcctggtcaaggactac |
| | chain | | Region | ttccccgaaccagtgacggtgtcgtggaactcaggcgccctgaccagc |
| | constant | | Nucleotide | ggcgtgcacaccttcccggctgtcctacagtcctcaggactctactcc |
| | region | | Sequence- | ctcagcagcgtggtgaccgtgccctccagcagcttgggcacgaagacc |
| | | | Synthetic | tacacctgcaacgtagatcacaagcccagcaacaccaaggggacaag |
| | | | Version D | agagttgagtccaaatatggtcccccatgcccaccatgcccagcgcct |
| | | | | ccagttgcggggggaccatcagtcttcctgttccccccaaaacccaag |
| | | | | gacactctcatgatctcccggacccctgaggtcacgtgcgtggtggtg |
| | | | | gacgtgagccaggaagaccccgaggtccagttcaactggtacgtggat |
| | | | | ggcgtggaggtgcataatgccaagacaaagccgcgggaggagcagttc |
| | | | | aacagcacgtaccgtgtggtcagcgtcctcaccgtcctgcaccaggac |
| | | | | tggctgaacggcaaggagtacaagtgcaaggtctccaacaaaggcctc |
| | | | | ccgtcatcgatcgagaaaaccatctccaaagccaaagggcagccccga |
| | | | | gagccacaggtgtacaccctgcccccatcccaggaggagatgaccaag |
| | | | | aaccaggtcagcctgacctgcctggtcaaaggcttctaccccagcgac |
| | | | | atcgccgtggagtgggagagcaatgggcagccggagaacaactacaag |
| | | | | accacgcctcccgtgctggactccgacggatccttcttcctctacagc |
| | | | | aggctaaccgtggacaagagcaggtggcaggaggggaatgtcttctca |
| | | | | tgctccgtgatgcatgaggctctgcacaaccactacacacagaagagc |
| | | | | ctctccctgtctctgggtaaa |
| 203 | | | Heavy Chain | ASTKGPSVFPLAPCSRSTSESTAALGCLVKDYFPEPVTVSWNSGALTS |
| | | | Constant | GVHTFPAVLQSSGLYSLSSVVTVPSSSLGTKTYTCNVDHKPSNTKVDK |
| | | | Region | RVESKYGPPCPPCPAPPVAGGPSVFLFPPKPKDTLMISRTPEVTCVVV |
| | | | Amino Acid | DVSQEDPEVQFNWYVDGVEVHNAKTKPREEQFNSTYRVVSVLTVLHQD |
| | | | Sequence- | WLNGKEYKCKVSNKGLPSSIEKTISKAKGQPREPQVYTLPPSQEEMTK |
| | | | encoded by | NQVSLTCLVKGFYPSDIAVEWESNGQPENNYKTTPPVLDSDGSFFLYS |
| | | | Synthetic | RLTVDKSRWQEGNVFSCSVMHEALHNHYTQKSLSLSLGK |
| | | | Version D | |
| 204 | Disabled | Disabled | Heavy Chain | gcctccaccaagggcccatcggtcttccccctggcaccctcctccaag |
| | Human | IGHG1 | Constant | agcacctctgggggcacagcggccctgggctgcctggtcaaggactac |
| | IgG1 | | Region | ttccccgaaccggtgacggtgtcgtggaactcaggcgccctgaccagc |
| | heavy | | Nucleotide | ggcgtgcacaccttcccggctgtcctacagtcctcaggactctactcc |
| | chain | | Sequence | ctcagcagcgtggtgaccgtgccctccagcagcttgggcacccagacc |
| | constant | | | tacatctgcaacgtgaatcacaagcccagcaacaccaaggtggacaag |
| | region | | | aaagtggagcccaaatcttgtgacaaaactcacacatgcccaccgtgc |
| | | | | ccagcacctgaactcgcgggggcaccgtcagtcttcctcttcccccca |
| | | | | aaacccaaggacaccctcatgatctcccggacccctgaggtcacatgc |
| | | | | gtggtggtggacgtgagccacgaagaccctgaggtcaagttcaactgg |
| | | | | tacgtggacggcgtggaggtgcataatgccaagacaaagccgcgggag |
| | | | | gagcagtacaacagcacgtaccgtgtggtcagcgtcctcaccgtcctg |
| | | | | caccaggactggctgaatggcaaggagtacaagtgcaaggtctccaac |
| | | | | aaagccctcccagcccccatcgagaaaaccatctccaaagccaaaggg |
| | | | | cagccccgagaaccacaggtgtacaccctgcccccatcccgggatgag |
| | | | | ctgaccaagaaccaggtcagcctgacctgcctggtcaaaggcttctat |
| | | | | cccagcgacatcgccgtggagtgggagagcaatgggcagccggagaac |
| | | | | aactacaagaccacgcctcccgtgctggactccgacggctccttcttc |
| | | | | ctctacagcaagctcaccgtggacaagagcaggtggcagcaggggaac |
| | | | | gtcttctcatgctccgtgatgcatgaggctctgcacaaccactacacg |
| | | | | cagaagagcctctccctgtctccgggtaaa |
| 205 | | | Heavy Chain | ASTKGPSVFPLAPSSKSTSGGTAALGCLVKDYFPEPVTVSWNSGALTS |
| | | | Constant | GVHTFPAVLQSSGLYSLSSVVTVPSSSLGTQTYICNVNHKPSNTKVDK |
| | | | Region | KVEPKSCDKTHTCPPCPAPELAGAPSVFLFPPKPKDTLMISRTPEVTC |
| | | | Amino Acid | VVVDVSHEDPEVKFNWYVDGVEVHNAKTKPREEQYNSTYRVVSVLTVL |
| | | | Sequence | HQDWLNGKEYKCKVSNKALPAPIEKTISKAKGQPREPQVYTLPPSRDE |
| | | | (Two | LTKNQVSLTCLVKGFYPSDIAVEWESNGQPENNYKTTPPVLDSDGSFF |
| | | | residues | LYSKLTVDKSRWQQGNVFSCSVMHEALHNHYTQKSLSLSPGK |
| | | | that differ | |
| | | | from the | |
| | | | wild-type | |
| | | | sequence | |
| | | | are | |
| | | | identified | |
| | | | in bold) | |
| 206 | Human Cκ | IGKC*01 | Cκ Light | cgtacggtggccgctccctccgtgttcatcttcccaccttccgacgag |
| | constant | | Chain | cagctgaagtccggcaccgcttctgtcgtgtgcctgctgaacaacttc |
| | region | | Constant | tacccccgcgaggccaaggtgcagtggaaggtggacaacgccctgcag |
| | | | Region | tccggcaactcccaggaatccgtgaccgagcaggactccaaggacagc |
| | | | Nucleotide | acctactccctgtcctccaccctgaccctgtccaaggccgactacgag |
| | | | Sequence | aagcacaaggtgtacgcctgcgaagtgacccaccagggcctgtctagc |
| | | | | cccgtgaccaagtctttcaaccggggcgagtgt |
| 207 | | | Cκ Light | RTVAAPSVFIFPPSDEQLKSGTASVVCLLNNFYPREAKVQWKVDNALQ |
| | | | Chain | SGNSQESVTEQDSKDSTYSLSSTLTLSKADYEKHKVYACEVTHQGLSS |
| | | | Constant | PVTKSFNRGEC |
| | | | Region | |
| | | | Amino Acid | |
| | | | Sequence | |
| 208 | Human Cκ | IGKC*02 | Cκ Light | cgaactgtggctgcaccatctgtcttcatcttcccgccatctgatgag |
| | constant | | Chain | cagttgaaatctggaactgcctctgttgtgtgcctgctgaataacttc |
| | region | | Constant | tatcccagagaggccaaagtacagtggaaggtggataacgccctccaa |
| | | | Region | tcgggtaactcccaggagagtgtcacagagcaggagagcaaggacagc |
| | | | Nucleotide | acctacagcctcagcagcaccctgacgctgagcaaagcagactacgag |
| | | | Sequence | aaacacaaagtctacgccggcgaagtcacccatcagggcctgagctcg |
| | | | | cccgtcacaaagagcttcaacaggggagagtgt |
| 209 | | | Cκ Light | RTVAAPSVFIFPPSDEQLKSGTASVVCLLNNFYPREAKVQWKVDNALQ |
| | | | Chain | SGNSQESVTEQESKDSTYSLSSTLTLSKADYEKHKVYAGEVTHQGLSS |
| | | | Constant | PVTKSFNRGEC |
| | | | Region | |
| | | | Amino Acid | |
| | | | Sequence | |
| 210 | Human Cκ | IGKC*03 | Cκ Light | cgaactgtggctgcaccatctgtcttcatcttcccgccatctgatgag |
| | constant | | Chain | cagttgaaatctggaactgcctctgttgtgtgcctgctgaataacttc |
| | region | | Constant | tatcccagagaggccaaagtacagcggaaggtggataacgccctccaa |
| | | | Region | tcgggtaactcccaggagagtgtcacagagcaggagagcaaggacagc |
| | | | Nucleotide | acctacagcctcagcagcaccctgacgctgagcaaagcagactacgag |
| | | | Sequence | aaacacaaagtctacgcctgcgaagtcacccatcagggcctgagctcg |
| | | | | cccgtcacaaagagcttcaacaggggagagtgt |
| 211 | | | Cκ Light | RTVAAPSVFIFPPSDEQLKSGTASVVCLLNNFYPREAKVQRKVDNALQ |
| | | | Chain | SGNSQESVTEQESKDSTYSLSSTLTLSKADYEKHKVYACEVTHQGLSS |
| | | | Constant | PVTKSFNRGEC |
| | | | Region | |
| | | | Amino Acid | |
| | | | Sequence | |
| 212 | Human Cκ | IGKC*04 | Cκ Light | cgaactgtggctgcaccatctgtcttcatcttcccgccatctgatgag |
| | constant | | Chain | cagttgaaatctggaactgcctctgttgtgtgcctgctgaataacttc |
| | region | | Constant | tatcccagagaggccaaagtacagtggaaggtggataacgccctccaa |
| | | | Region | tcgggtaactcccaggagagtgtcacagagcaggacagcaaggacagc |
| | | | Nucleotide | acctacagcctcagcagcaccctgacgctgagcaaagcagactacgag |
| | | | Sequence | aaacacaaactctacgcctgcgaagtcacccatcagggcctgagctcg |
| | | | | cccgtcacaaagagcttcaacaggggagagtgt |
| 213 | | | Cκ Light | RTVAAPSVFIFPPSDEQLKSGTASVVCLLNNFYPREAKVQWKVDNALQ |
| | | | Chain | SGNSQESVTEQDSKDSTYSLSSTLTLSKADYEKHKLYACEVTHQGLSS |
| | | | Constant | PVTKSFNRGEC |
| | | | Region | |
| | | | Amino Acid | |
| | | | Sequence | |
| 214 | Human Cκ | IGKC*05 | Cκ Light | cgaactgtggctgcaccatctgtcttcatcttcccgccatctgatgag |
| | constant | | Chain | cagttgaaatctggaactgcctctgttgtgtgcctgctgaataacttc |
| | region | | Constant | tatcccagagaggccaaagtacagtggaaggtggataacgccctccaa |
| | | | Region | tcgggtaactcccaggagagtgtcacagagcaggacagcaaggacagc |
| | | | Nucleotide | acctacagcctcagcaacaccctgacgctgagcaaagcagactacgag |
| | | | Sequence | aaacacaaagtctacgcctgcgaagtcacccatcagggcctgagctcg |
| | | | | cccgtcacaaagagcttcaacaggggagagtgc |
| 215 | | | CK Light | RTVAAPSVFIFPPSDEQLKSGTASVVCLLNNFYPREAKVQWKVDNALQ |
| | | | Chain | SGNSQESVTEQDSKDSTYSLSNTLTLSKADYEKHKVYACEVTHQGLSS |
| | | | Constant | PVTKSFNRGEC |
| | | | Region | |
| | | | Amino Acid | |
| | | | Sequence | |
| 216 | Human Cλ | IGCA1*01 | Cλ Light | cccaaggccaaccccacggtcactctgttcccgccctcctctgaggag |
| | constant | | Chain | ctccaagccaacaaggccacactagtgtgtctgatcagtgacttctac |
| | region | | Constant | ccgggagctgtgacagtggcttggaaggcagatggcagccccgtcaag |
| | | | Region | gcgggagtggagacgaccaaaccctccaaacagagcaacaacaagtac |
| | | | Nucleotide | gcggccagcagctacctgagcctgacgcccgagcagtggaagtcccac |
| | | | Sequence | agaagctacagctgccaggtcacgcatgaagggagcaccgtggagaag |
| | | | | acagtggcccctacagaatgttca |
| 217 | | | Cλ Light | PKANPTVTLFPPSSEELQANKATLVCLISDFYPGAVTVAWKADGSPVK |
| | | | Chain | AGVETTKPSKQSNNKYAASSYLSLTPEQWKSHRSYSCQVTHEGSTVEK |
| | | | Constant | TVAPTECS |
| | | | Region | |
| | | | Amino Acid | |
| | | | Sequence | |
| 218 | Human Cλ | IGCA1*02 | Cλ Light | ggtcagcccaaggccaaccccactgtcactctgttcccgccctcctct |
| | constant | | Chain | gaggagctccaagccaacaaggccacactagtgtgtctgatcagtgac |
| | region | | Constant | ttctacccgggagctgtgacagtggcctggaaggcagatggcagcccc |
| | | | Region | gtcaaggcgggagtggagaccaccaaaccctccaaacagagcaacaac |
| | | | Nucleotide | aagtacgcggccagcagctacctgagcctgacgcccgagcagtggaag |
| | | | Sequence | tcccacagaagctacagctgccaggtcacgcatgaagggagcaccgtg |
| | | | | gagaagacagtggcccctacagaatgttca |
| 219 | | | Cλ Light | GQPKANPTVTLFPPSSEELQANKATLVCLISDFYPGAVTVAWKADGSP |
| | | | Chain | VKAGVETTKPSKQSNNKYAASSYLSLTPEQWKSHRSYSCQVTHEGSTV |
| | | | Constant | EKTVAPTECS |
| | | | Region | |
| | | | Amino Acid | |
| | | | Sequence | |
| 220 | Human Cλ | IGCA2*01 | Cλ Light | ggtcagcccaaggccaaccccactgtcactctgttcccgccctcctct |
| | constant | | Chain | gaggagctccaagccaacaaggccacactagtgtgtctgatcagtgac |
| | region | | Constant | ttctacccgggagctgtgacagtggcctggaaggcagatggcagcccc |
| | | | Region | gtcaaggcgggagtggagaccaccaaaccctccaaacagagcaacaac |
| | | | Nucleotide | aagtacgcggccagcagctacctgagcctgacgcccgagcagtggaag |
| | | | Sequence- | tcccacagaagctacagctgccaggtcacgcatgaagggagcaccgtg |
| | | | Version A | gagaagacagtggcccctacagaatgttca |
| 221 | | | Cλ Light | ggccagcctaaggccgctccttctgtgaccctgttccccccatcctcc |
| | | | Chain | gaggaactgcaggctaacaaggccaccctcgtgtgcctgatcagcgac |
| | | | Constant | ttctaccctggcgccgtgaccgtggcctggaaggctgatagctctcct |
| | | | Region | gtgaaggccggcgtggaaaccaccaccccttccaagcagtccaacaac |
| | | | Nucleotide | aaatacgccgcctcctcctacctgtccctgacccctgagcagtggaag |
| | | | Sequence- | tcccaccggtcctacagctgccaagtgacccacgagggctccaccgtg |
| | | | Version B | gaaaagaccgtggctcctaccgagtgctcc |
| 222 | | | Cλ Light | ggccagcctaaagctgcccccagcgtcaccctgtttcctccctccagc |
| | | | Chain | gaggagctccaggccaacaaggccaccctcgtgtgcctgatctccgac |
| | | | Constant | ttctatcccggcgctgtgaccgtggcttggaaagccgactccagccct |
| | | | Region | gtcaaagccggcgtggagaccaccacaccctccaagcagtccaacaac |
| | | | Nucleotide | aagtacgccgcctccagctatctctccctgacccctgagcagtggaag |
| | | | Sequence- | tcccaccggtcctactcctgtcaggtgacccacgagggctccaccgtg |
| | | | Version C | gaaaagaccgtcgcccccaccgagtgctcc |
| 223 | | | Cλ Light | GQPKANPTVTLFPPSSEELQANKATLVCLISDFYPGAVTVAWKADGSP |
| | | | Chain | VKAGVETTKPSKQSNNKYAASSYLSLTPEQWKSHRSYSCQVTHEGSTV |
| | | | Constant | EKTVAPTECS |
| | | | Region | |
| | | | Amino Acid | |
| | | | Sequence- | |
| | | | Encoded by | |
| | | | Version A, | |
| | | | B & C | |
| 224 | Human Cλ | IGCA2*02 | Cλ Light | ggtcagcccaaggctgccccctcggtcactctgttcccgccctcctct |
| | constant | & | Chain | gaggagcttcaagccaacaaggccacactggtgtgtctcataagtgac |
| | region | IGLC2*03 | Constant | ttctacccgggagccgtgacagtggcctggaaggcagatagcagcccc |
| | | | Region | gtcaagggggagtggagaccaccacaccctccaaacaaagcaacaac |
| | | | Nucleotide | aagtacgcggccagcagctatctgagcctgacgcctgagcagtggaag |
| | | | Sequence | tcccacagaagctacagctgccaggtcacgcatgaagggagcaccgtg |
| | | | | gagaagacagtggcccctacagaatgttca |
| 225 | | | Cλ Light | GQPKAAPSVTLFPPSSEELQANKATLVCLISDFYPGAVTVAWKADSSP |
| | | | Chain | VKAGVETTTPSKQSNNKYAASSYLSLTPEQWKSHRSYSCQVTHEGSTV |
| | | | Constant | EKTVAPTECS |
| | | | Region | |
| | | | Amino Acid | |
| | | | Sequence | |
| 226 | Human Cλ | IGCA3*01 | Cλ Light | cccaaggctgccccctcggtcactctgttcccaccctcctctgaggag |
| | constant | | Chain | cttcaagccaacaaggccacactggtgtgtctcataagtgacttctac |
| | region | | Constant | ccgggagccgtgacagttgcctggaaggcagatagcagccccgtcaag |
| | | | Region | gcgggggtggagaccaccacaccctocaaacaaagcaacaacaagtac |
| | | | Nucleotide | gcggccagcagctacctgagcctgacgcctgagcagtggaagtcccac |
| | | | Sequence | aaaagctacagctgccaggtcacgcatgaagggagcaccgtggagaag |
| | | | | acagttgcccctacggaatgttca |
| 227 | | | Cλ Light | PKAAPSVTLFPPSSEELQANKATLVCLISDFYPGAVTVAWKADSSPVK |
| | | | Chain | AGVETTTPSKQSNNKYAASSYLSLTPEQWKSHKSYSCQVTHEGSTVEK |
| | | | Constant | TVAPTECS |
| | | | Region | |
| | | | Amino Acid | |
| | | | Sequence | |
| 228 | Human Cλ | IGCA3*02 | Cλ Light | ggtcagcccaaggctgccccctcggtcactctgttcccaccctcctct |
| | constant | | Chain | gaggagcttcaagccaacaaggccacactggtgtgtctcataagtgac |
| | region | | Constant | ttctacccggggccagtgacagttgcctggaaggcagatagcagcccc |
| | | | Region | gtcaaggcgggggtggagaccaccacaccctccaaacaaagcaacaac |
| | | | Nucleotide | aagtacgcggccagcagctacctgagcctgacgcctgagcagtggaag |
| | | | Sequence | tcccacaaaagctacagctgccaggtcacgcatgaagggagcaccgtg |
| | | | | gagaagacagtggcccctacggaatgttca |
| 229 | | | Cλ Light | GQPKAAPSVTLFPPSSEELQANKATLVCLISDFYPGPVTVAWKADSSP |
| | | | Chain | VKAGVETTTPSKQSNNKYAASSYLSLTPEQWKSHKSYSCQVTHEGSTV |
| | | | Constant | EKTVAPTECS |
| | | | Region | |
| | | | Amino Acid | |
| | | | Sequence | |
| 230 | Human Cλ | IGCA3*03 | Cλ Light | ggtcagcccaaggctgccccctcggtcactctgttcccaccctcctct |
| | constant | | Chain | gaggagcttcaagccaacaaggccacactggtgtgtctcataagtgac |
| | region | | Constant | ttctacccgggagccgtgacagtggcctggaaggcagatagcagcccc |
| | | | Region | gtcaaggcgggagtggagaccaccacaccctccaaacaaagcaacaac |
| | | | Nucleotide | aagtacgcggccagcagctacctgagcctgacgcctgagcagtggaag |
| | | | Sequence | tcccacaaaagctacagctgccaggtcacgcatgaagggagcaccgtg |
| | | | | gagaagacagtggcccctacagaatgttca |
| 231 | | | Cλ Light | GQPKAAPSVTLFPPSSEELQANKATLVCLISDFYPGAVTVAWKADSSP |
| | | | Chain | VKAGVETTTPSKQSNNKYAASSYLSLTPEQWKSHKSYSCQVTHEGSTV |
| | | | Constant | EKTVAPTECS |
| | | | Region | |
| | | | Amino Acid | |
| | | | Sequence | |
| 232 | Human Cλ | IGCA3*04 | Cλ Light | ggtcagcccaaggctgccccctcggtcactctgttcccgccctcctct |
| | constant | | Chain | gaggagcttcaagccaacaaggccacactggtgtgtctcataagtgac |
| | region | | Constant | ttctacccgggagccgtgacagtggcctggaaggcagatagcagcccc |
| | | | Region | gtcaaggcgggagtggagaccaccacaccctccaaacaaagcaacaac |
| | | | Nucleotide | aagtacgcggccagcagctacctgagcctgacgcctgagcagtggaag |
| | | | Sequence | tcccacagaagctacagctgccaggtcacgcatgaagggagcaccgtg |
| | | | | gagaagacagtggcccctacagaatgttca |
| 233 | | | Cλ Light | GQPKAAPSVTLFPPSSEELQANKATLVCLISDFYPGAVTVAWKADSSP |
| | | | Chain | VKAGVETTTPSKQSNNKYAASSYLSLTPEQWKSHRSYSCQVTHEGSTV |
| | | | Constant | EKTVAPTECS |
| | | | Region | |
| | | | Amino Acid | |
| | | | Sequence | |
| 234 | Human Cλ | IGCA6*01 | Cλ Light | ggtcagcccaaggctgccccatcggtcactctgttcccgccctcctct |
| | constant | | Chain | gaggagcttcaagccaacaaggccacactggtgtgcctgatcagtgac |
| | region | | Constant | ttctacccgggagctgtgaaagtggcctggaaggcagatggcagcccc |
| | | | Region | gtcaacacgggagtggagaccaccacaccctccaaacagagcaacaac |
| | | | Nucleotide | aagtacgcggccagcagctacctgagcctgacgcctgagcagtggaag |
| | | | Sequence | tcccacagaagctacagctgccaggtcacgcatgaagggagcaccgtg |
| | | | | gagaagacagtggcccctgcagaatgttca |
| 235 | | | Cλ Light | GQPKAAPSVTLFPPSSEELQANKATLVCLISDFYPGAVKVAWKADGSP |
| | | | Chain | VNTGVETTTPSKQSNNKYAASSYLSLTPEQWKSHRSYSCQVTHEGSTV |
| | | | Constant | EKTVAPAECS |
| | | | Region | |
| | | | Amino Acid | |
| | | | Sequence | |
| 236 | Human Cλ | IGLC7*01 | Cλ Light | ggtcagcccaaggctgccccatcggtcactctgttcccaccctcctct |
| | constant | & | Chain | gaggagcttcaagccaacaaggccacactggtgtgtctcgtaagtgac |
| | region | IGCA7*02 | Constant | ttctacccgggagccgtgacagtggcctggaaggcagatggcagcccc |
| | | | Region | gtcaaggtgggagtggagaccaccaaaccctccaaacaaagcaacaac |
| | | | Nucleotide | aagtatgcggccagcagctacctgagcctgacgcccgagcagtggaag |
| | | | Sequence | tcccacagaagctacagctgccgggtcacgcatgaagggagcaccgtg |
| | | | | gagaagacagtggcccctgcagaatgctct |
| 237 | | | Cλ Light | GQPKAAPSVTLFPPSSEELQANKATLVCLVSDFYPGAVTVAWKADGSP |
| | | | Chain | VKVGVETTKPSKQSNNKYAASSYLSLTPEQWKSHRSYSCRVTHEGSTV |
| | | | Constant | EKTVAPAECS |
| | | | Region | |
| | | | Amino Acid | |
| | | | Sequence | |

| 238 | 413G05- | Amino acid sequence | | GFTFSDYY |
|---|---|---|---|---|
| | CDRH1 | of CDRH1 of 413G05 | | |
| | (IMGT) | using IMGT | | |
| 239 | 413G05- | Amino acid sequence | | ISTSGSTI |
| | CDRH2 | of CDRH2 of 413G05 | | |
| | (IMGT) | using IMGT | | |
| 240 | 413G05- | Amino acid sequence | | ARGITGTNFYHYGLGV |
| | CDRH3 | of CDRH3 of 413G05 | | |
| | (IMGT) | using IMGT | | |
| 241 | 413G05- | Amino acid sequence | | DYYMS |
| | CDRH1 | of CDRH1 of 413G05 | | |
| | (Kabat) | using Kabat | | |
| 242 | 413G05- | Amino acid sequence | | YISTSGSTIYYADSVKG |
| | CDRH2 | of CDRH2 of 413G05 | | |
| | (Kabat) | using Kabat | | |
| 243 | 413G05- | Amino acid sequence | | GITGTNFYHYGLGV |
| | CDRH3 | of CDRH3 of 413G05 | | |
| | (Kabat) | using Kabat | | |
| 244 | 413G05- | Amino acid sequence | | QVQLVESGGGLVKPGGSLRLSCAASGFTFSDYYMSWIRQVPGKGLEWV |
| | Heavy | of V_{H} of 413G05 | | SYISTSGSTIYYADSVKGRFTISRDNAKNSLYLQMNSLRAEDAAVYHC |
| | chain | | | ARGITGTNFYHYGLGVWGQGTTVTVSS |
| | variable | | | |
| | region | | | |
| 245 | 413G05- | Nucleic acid | | CAGGTGCAGCTGGTGGAGTCTGGGGGAGGCTTGGTCAAGCCTGGAGGG |
| | Heavy | sequence of V_{H} of | | TCCCTGAGACTCTCCTGTGCAGCCTCTGGATTCACCTTCAGTGACTAC |
| | chain | 413G05 | | TACATGAGCTGGATCCGCCAGGTTCCAGGGAAGGGGCTGGAGTGGGTT |
| | variable | | | TCATACATTAGTACTAGTGGTAGTACCATATACTACGCAGACTCTGTG |
| | region | | | AAGGGCCGATTCACCATCTCCAGGGACAACGCCAAGAACTCACTGTAT |
| | | | | CTACAAATGAACAGCCTGAGAGCCGAGGACGCGGCCGTGTATCACTGT |
| | | | | GCGAGAGGTATAACTGGAACTAACTTCTACCACTACGGTTTGGGCGTC |
| | | | | TGGGGCCAAGGGACCACGGTCACCGTCTCCTCAG |
| 246 | 413G05- | Amino acid sequence | | QVQLVESGGGLVKPGGSLRLSCAASGFTFSDYYMSWIRQVPGKGLEWV |
| | full | of 413G05 heavy | | SYISTSGSTIYYADSVKGRFTISRDNAKNSLYLQMNSLRAEDAAVYHC |
| | heavy | chain | | ARGITGTNFYHYGLGVWGQGTTVTVSSASTKGPSVFPLAPSSKSTSGG |
| | chain | | | TAALGCLVKDYFPEPVTVSWNSGALTSGVHTFPAVLQSSGLYSLSSVV |
| | sequence | | | TVPSSSLGTQTYICNVNHKPSNTKVDKKVEPKSCDKTHTCPPCPAPEL |
| | | | | LGGPSVFLFPPKPKDTLMISRTPEVTCVVVDVSHEDPEVKFNWYVDGV |
| | | | | EVHNAKTKPREEQYNSTYRVVSVLTVLHQDWLNGKEYKCKVSNKALPA |
| | | | | PIEKTISKAKGQPREPQVYTLPPSRDELTKNQVSLTCLVKGFYPSDIA |
| | | | | VEWESNGQPENNYKTTPPVLDSDGSFFLYSKLTVDKSRWQQGNVFSCS |
| | | | | VMHEALHNHYTQKSLSLSPGK |
| 247 | 413G05- | Nucleic acid | | CAGGTGCAGCTGGTGGAGTCTGGGGGAGGCTTGGTCAAGCCTGGAGGG |
| | full | sequence of 413G05 | | TCCCTGAGACTCTCCTGTGCAGCCTCTGGATTCACCTTCAGTGACTAC |
| | heavy | heavy chain | | TACATGAGCTGGATCCGCCAGGTTCCAGGGAAGGGGCTGGAGTGGGTT |
| | chain | | | TCATACATTAGTACTAGTGGTAGTACCATATACTACGCAGACTCTGTG |
| | sequence | | | AAGGGCCGATTCACCATCTCCAGGGACAACGCCAAGAACTCACTGTAT |
| | | | | CTACAAATGAACAGCCTGAGAGCCGAGGACGCGGCCGTGTATCACTGT |
| | | | | GCGAGAGGTATAACTGGAACTAACTTCTACCACTACGGTTTGGGCGTC |
| | | | | TGGGGCCAAGGGACCACGGTCACCGTCTCCTCAGCCAGCACCAAGGGC |
| | | | | CCCTCTGTGTTCCCTCTGGCCCCTTCCAGCAAGTCCACCTCTGGCGGA |
| | | | | ACAGCCGCTCTGGGCTGCCTCGTGAAGGACTACTTCCCCGAGCCTGTG |
| | | | | ACCGTGTCCTGGAACTCTGGCGCTCTGACCAGCGGAGTGCACACCTTC |
| | | | | CCTGCTGTGCTGCAGTCCTCCGGCCTGTACTCCCTGTCCTCCGTCGTG |
| | | | | ACCGTGCCTTCCAGCTCTCTGGGCACCCAGACCTACATCTGCAACGTG |
| | | | | AACCACAAGCCCTCCAACACCAAGGTGGACAAGAAGGTGGAACCCAAG |
| | | | | TCCTGCGACAAGACCCACACCTGTCCCCCTTGTCCTGCCCCTGAACTG |
| | | | | CTGGGCGGACCTTCCGTGTTCCTGTTCCCCCCAAAGCCCAAGGACACC |
| | | | | CTGATGATCTCCCGGACCCCCGAAGTGACCTGCGTGGTGGTGGATGTG |
| | | | | TCCCACGAGGACCCTGAAGTGAAGTTCAATTGGTACGTGGACGGCGTG |
| | | | | GAAGTGCACAACGCCAAGACCAAGCCTAGAGAGGAACAGTACAACTCC |
| | | | | ACCTACCGGGTGGTGTCCGTGCTGACCGTGCTGCACCAGGATTGGCTG |
| | | | | AACGGCAAAGAGTACAAGTGCAAGGTGTCCAACAAGGCCCTGCCTGCC |
| | | | | CCCATCGAAAAGACCATCTCCAAGGCCAAGGGCCAGCCCCGGGAACCC |
| | | | | CAGGTGTACACACTGCCCCCTAGCAGGGACGAGCTGACCAAGAACCAG |
| | | | | GTGTCCCTGACCTGTCTCGTGAAAGGCTTCTACCCCTCCGATATCGCC |
| | | | | GTGGAATGGGAGTCCAACGGCCAGCCTGAGAACAACTACAAGACCACC |
| | | | | CCCCCTGTGCTGGACTCCGACGGCTCATTCTTCCTGTACAGCAAGCTG |
| | | | | ACAGTGGACAAGTCCCGGTGGCAGCAGGGCAACGTGTTCTCCTGCTCC |
| | | | | GTGATGCACGAGGCCCTGCACAACCACTACACCCAGAAGTCCCTGTCC |
| | | | | CTGAGCCCCGGCAAG |
| 248 | 413G05- | Amino acid sequence | | QGINSW |
| | CDRL1 | of CDRL1 of 413G05 | | |
| | (IMGT) | using IMGT | | |
| 249 | 413G05- | Amino acid sequence | | AAS |
| | CDRL2 | of CDRL2 of 413G05 | | |
| | (IMGT) | using IMGT | | |
| 250 | 413G05- | Amino acid sequence | | QQVNSFPLT |
| | CDRL3 | of CDRL3 of 413G05 | | |
| | (IMGT) | using IMGT | | |
| 251 | 413G05- | Amino acid sequence | | RASQGINSWLA |
| | CDRL1 | of CDRL1 of 413G05 | | |
| | (Kabat) | using Kabat | | |
| 252 | 413G05- | Amino acid sequence | | AASTLOS |
| | CDRL2 | of CDRL2 of 413G05 | | |
| | (Kabat) | using Kabat | | |
| 253 | 413G05- | Amino acid sequence | | QQVNSFPLT |
| | CDRL3 | of CDRL3 of 413G05 | | |
| | (Kabat) | using Kabat | | |
| 254 | 413G05- | Amino acid sequence | | DIQMTQSPSSVSASVGDRVTITCRASQGINSWLAWYQQKPGKAPKLLI |
| | Light | of V_{L} of 413G05 | | YAASTLQSGVPSRFSGSGSGADFTLTISSLOPEDFATYYCQQVNSFPL |
| | chain | | | TFGGGTKVEIK |
| | variable | | | |
| | region | | | |
| 255 | 413G05- | Nucleic acid | | GACATCCAGATGACCCAGTCTCCATCTTCCGTGTCTGCATCTGTAGGA |
| | Light | sequence of V_{L} of | | GACAGAGTCACCATCACTTGTCGGGCGAGTCAGGGTATTAACAGCTGG |
| | chain | 413G05 | | TTAGCCTGGTATCAGCAGAAACCAGGGAAAGCCCCTAAGCTCCTGATC |
| | variable | | | TATGCTGCATCCACTTTGCAAAGTGGGGTCCCATCAAGGTTCAGCGGC |
| | region | | | AGTGGGTCTGGGGCAGATTTCACTCTCACCATCAGCAGCCTGCAGCCT |
| | | | | GAAGATTTTGCAACTTACTATTGTCAACAGGTTAACAGTTTCCCGCTC |
| | | | | ACTTTCGGCGGAGGGACCAAGGTGGAGATCAAAC |
| 256 | 413G05- | Amino acid sequence | | DIQMTQSPSSVSASVGDRVTITCRASQGINSWLAWYQQKPGKAPKLLI |
| | full | of 413G05 light | | YAASTLQSGVPSRFSGSGSGADFTLTISSLQPEDFATYYCQQVNSFPL |
| | light | chain | | TFGGGTKVEIKRTVAAPSVFIFPPSDEQLKSGTASVVCLLNNFYPREA |
| | chain | | | KVQWKVDNALQSGNSQESVTEQDSKDSTYSLSSTLTLSKADYEKHKVY |
| | sequence | | | ACEVTHQGLSSPVTKSFNRGEC |
| 257 | 413G05- | Nucleic acid | | GACATCCAGATGACCCAGTCTCCATCTTCCGTGTCTGCATCTGTAGGA |
| | full | sequence of 413G05 | | GACAGAGTCACCATCACTTGTCGGGCGAGTCAGGGTATTAACAGCTGG |
| | light | light chain | | TTAGCCTGGTATCAGCAGAAACCAGGGAAAGCCCCTAAGCTCCTGATC |
| | chain | | | TATGCTGCATCCACTTTGCAAAGTGGGGTCCCATCAAGGTTCAGCGGC |
| | sequence | | | AGTGGGTCTGGGGCAGATTTCACTCTCACCATCAGCAGCCTGCAGCCT |
| | | | | GAAGATTTTGCAACTTACTATTGTCAACAGGTTAACAGTTTCCCGCTC |
| | | | | ACTTTCGGCGGAGGGACCAAGGTGGAGATCAAACGTACGGTGGCCGCT |
| | | | | CCCTCCGTGTTCATCTTCCCACCTTCCGACGAGCAGCTGAAGTCCGGC |
| | | | | ACCGCTTCTGTCGTGTGCCTGCTGAACAACTTCTACCCCCGCGAGGCC |
| | | | | AAGGTGCAGTGGAAGGTGGACAACGCCCTGCAGTCCGGCAACTCCCAG |
| | | | | GAATCCGTGACCGAGCAGGACTCCAAGGACAGCACCTACTCCCTGTCC |
| | | | | TCCACCCTGACCCTGTCCAAGGCCGACTACGAGAAGCACAAGGTGTAC |
| | | | | GCCTGCGAAGTGACCCACCAGGGCCTGTCTAGCCCCGTGACCAAGTCT |
| | | | | TTCAACCGGGGCGAGTGT |
| 258 | 413F09- | Amino acid sequence | | GFTFSYYA |
| | CDRH1 | of CDRH1 of 413F09 | | |
| | (IMGT) | using IMGT | | |
| 259 | 413F09- | Amino acid sequence | | ISGGGGNT |
| | CDRH2 | of CDRH2 of 413F09 | | |
| | (IMGT) | using IMGT | | |
| 260 | 413F09- | Amino acid sequence | | AKDRMKQLVRAYYFDY |
| | CDRH3 | of CDRH3 of 413F09 | | |
| | (IMGT) | using IMGT | | |
| 261 | 413F09- | Amino acid sequence | | YYAMS |
| | CDRH1 | of CDRH1 of 413F09 | | |
| | (Kabat) | using Kabat | | |
| 262 | 413F09- | Amino acid sequence | | TISGGGGNTHYADSVKG |
| | CDRH2 | of CDRH2 of 413F09 | | |
| | (Kabat) | using Kabat | | |
| 263 | 413F09- | Amino acid sequence | | DRMKQLVRAYYFDY |
| | CDRH3 | of CDRH3 of 413F09 | | |
| | (Kabat) | using Kabat | | |
| 264 | 413F09- | Amino acid sequence | | EVPLVESGGGLVQPGGSLRLSCAASGFTFSYYAMSWVRQAPGKGLDWV |
| | Heavy | of V_{H} of 413F09 | | STISGGGGNTHYADSVKGRFTISRDNSKNTLYLHMNSLRAEDTAVYYC |
| | chain | | | AKDRMKQLVRAYYFDYWGQGTLVTVSS |
| | variable | | | |
| | region | | | |
| 265 | 413F09- | Nucleic acid | | GAGGTGCCGCTGGTGGAGTCTGGGGGAGGCTTGGTACAGCCTGGGGGG |
| | Heavy | sequence of V_{H} of | | TCCCTGAGACTCTCCTGTGCAGCCTCTGGATTCACGTTTAGCTACTAT |
| | chain | 413F09 | | GCCATGAGCTGGGTCCGTCAGGCTCCAGGGAAGGGGCTGGACTGGGTC |
| | variable | | | TCAACTATTAGTGGTGGTGGTGGTAACACACACTACGCAGACTCCGTG |
| | region | | | AAGGGCCGATTCACTATATCCAGAGACAATTCCAAGAACACGCTGTAT |
| | | | | CTGCACATGAACAGCCTGAGAGCCGAAGACACGGCCGTCTATTACTGT |
| | | | | GCGAAGGATCGGATGAAACAGCTCGTCCGGGCCTACTACTTTGACTAC |
| | | | | TGGGGCCAGGGAACCCTGGTCACCGTCTCCTCAG |
| 266 | 413F09- | Amino acid sequence | | EVPLVESGGGLVQPGGSLRLSCAASGFTFSYYAMSWVRQAPGKGLDWV |
| | full | of 413F09 heavy | | STISGGGGNTHYADSVKGRFTISRDNSKNTLYLHMNSLRAEDTAVYYC |
| | heavy | chain | | AKDRMKQLVRAYYFDYWGQGTLVTVSSASTKGPSVFPLAPSSKSTSGG |
| | chain | | | TAALGCLVKDYFPEPVTVSWNSGALTSGVHTFPAVLQSSGLYSLSSVV |
| | sequence | | | TVPSSSLGTQTYICNVNHKPSNTKVDKKVEPKSCDKTHTCPPCPAPEL |
| | | | | LGGPSVFLFPPKPKDTLMISRTPEVTCVVVDVSHEDPEVKFNWYVDGV |
| | | | | EVHNAKTKPREEQYNSTYRVVSVLTVLHQDWLNGKEYKCKVSNKALPA |
| | | | | PIEKTISKAKGQPREPQVYTLPPSRDELTKNQVSLTCLVKGFYPSDIA |
| | | | | VEWESNGQPENNYKTTPPVLDSDGSFFLYSKLTVDKSRWQQGNVFSCS |
| | | | | VMHEALHNHYTQKSLSLSPGK |
| 267 | 413F09- | Nucleic acid | | GAGGTGCCGCTGGTGGAGTCTGGGGGAGGCTTGGTACAGCCTGGGGGG |
| | full | sequence of 413F09 | | TCCCTGAGACTCTCCTGTGCAGCCTCTGGATTCACGTTTAGCTACTAT |
| | heavy | heavy chain | | GCCATGAGCTGGGTCCGTCAGGCTCCAGGGAAGGGGCTGGACTGGGTC |
| | chain | | | TCAACTATTAGTGGTGGTGGTGGTAACACACACTACGCAGACTCCGTG |
| | sequence | | | AAGGGCCGATTCACTATATCCAGAGACAATTCCAAGAACACGCTGTAT |
| | | | | CTGCACATGAACAGCCTGAGAGCCGAAGACACGGCCGTCTATTACTGT |
| | | | | GCGAAGGATCGGATGAAACAGCTCGTCCGGGCCTACTACTTTGACTAC |
| | | | | TGGGGCCAGGGAACCCTGGTCACCGTCTCCTCAGCCAGCACCAAGGGC |
| | | | | CCCTCTGTGTTCCCTCTGGCCCCTTCCAGCAAGTCCACCTCTGGCGGA |
| | | | | ACAGCCGCTCTGGGCTGCCTCGTGAAGGACTACTTCCCCGAGCCTGTG |
| | | | | ACCGTGTCCTGGAACTCTGGCGCTCTGACCAGCGGAGTGCACACCTTC |
| | | | | CCTGCTGTGCTGCAGTCCTCCGGCCTGTACTCCCTGTCCTCCGTCGTG |
| | | | | ACCGTGCCTTCCAGCTCTCTGGGCACCCAGACCTACATCTGCAACGTG |
| | | | | AACCACAAGCCCTCCAACACCAAGGTGGACAAGAAGGTGGAACCCAAG |
| | | | | TCCTGCGACAAGACCCACACCTGTCCCCCTTGTCCTGCCCCTGAACTG |
| | | | | CTGGGCGGACCTTCCGTGTTCCTGTTCCCCCCAAAGCCCAAGGACACC |
| | | | | CTGATGATCTCCCGGACCCCCGAAGTGACCTGCGTGGTGGTGGATGTG |
| | | | | TCCCACGAGGACCCTGAAGTGAAGTTCAATTGGTACGTGGACGGCGTG |
| | | | | GAAGTGCACAACGCCAAGACCAAGCCTAGAGAGGAACAGTACAACTCC |
| | | | | ACCTACCGGGTGGTGTCCGTGCTGACCGTGCTGCACCAGGATTGGCTG |
| | | | | AACGGCAAAGAGTACAAGTGCAAGGTGTCCAACAAGGCCCTGCCTGCC |
| | | | | CCCATCGAAAAGACCATCTCCAAGGCCAAGGGCCAGCCCCGGGAACCC |
| | | | | CAGGTGTACACACTGCCCCCTAGCAGGGACGAGCTGACCAAGAACCAG |
| | | | | GTGTCCCTGACCTGTCTCGTGAAAGGCTTCTACCCCTCCGATATCGCC |
| | | | | GTGGAATGGGAGTCCAACGGCCAGCCTGAGAACAACTACAAGACCACC |
| | | | | CCCCCTGTGCTGGACTCCGACGGCTCATTCTTCCTGTACAGCAAGCTG |
| | | | | ACAGTGGACAAGTCCCGGTGGCAGCAGGGCAACGTGTTCTCCTGCTCC |
| | | | | GTGATGCACGAGGCCCTGCACAACCACTACACCCAGAAGTCCCTGTCC |
| | | | | CTGAGCCCCGGCAAG |
| 268 | 413F09- | Amino acid sequence | | QDISTY |
| | CDRL1 | of CDRL1 of 413F09 | | |
| | (IMGT) | using IMGT | | |
| 269 | 413F09- | Amino acid sequence | | GTS |
| | CDRL2 | of CDRL2 of 413F09 | | |
| | (IMGT) | using IMGT | | |
| 270 | 413F09- | Amino acid sequence | | QQLHTDPIT |
| | CDRL3 | of CDRL3 of 413F09 | | |
| | (IMGT) | using IMGT | | |
| 271 | 413F09- | Amino acid sequence | | WASQDISTYLG |
| | CDRL1 | of CDRL1 of 413F09 | | |
| | (Kabat) | using Kabat | | |
| 272 | 413F09- | Amino acid sequence | | GTSSLQS |
| | CDRL2 | of CDRL2 of 413F09 | | |
| | (Kabat) | using Kabat | | |
| 273 | 413F09- | Amino acid sequence | | QQLHTDPIT |
| | CDRL3 | of CDRL3 of 413F09 | | |
| | (Kabat) | using Kabat | | |
| 274 | 413F09- | Amino acid sequence | | DIQLTQSPSFLSASVGDRVTITCWASQDISTYLGWYQQKPGKAPKLLI |
| | Light | of V_{L} of 413F09 | | YGTSSLQSGVPSRFSGSGSGTEFTLTISSLQPEDFATYYCQQLHTDPI |
| | chain | | | TFGQGTRLEIK |
| | variable | | | |
| | region | | | |
| 275 | 413F09- | Nucleic acid | | GACATCCAGTTGACCCAGTCTCCATCCTTCCTGTCTGCATCTGTAGGA |
| | Light | sequence of V_{L} of | | GACAGAGTCACCATCACTTGCTGGGCCAGTCAGGACATTAGCACTTAT |
| | chain | 413F09 | | TTAGGCTGGTATCAGCAAAAACCAGGGAAAGCCCCTAAGCTCCTGATC |
| | variable | | | TATGGTACATCCAGTTTGCAAAGTGGGGTCCCATCAAGGTTCAGCGGC |
| | region | | | AGTGGATCTGGGACAGAATTCACTCTCACAATCAGCAGCCTGCAGCCT |
| | | | | GAAGATTTTGCAACTTATTACTGTCAACAGCTTCATACTGACCCGATC |
| | | | | ACCTTCGGCCAAGGGACACGACTGGAGATCAAAC |
| 276 | 413F09- | Amino acid sequence | | DIQLTQSPSFLSASVGDRVTITCWASQDISTYLGWYQQKPGKAPKLLI |
| | full | of 413F09 light | | YGTSSLQSGVPSRFSGSGSGTEFTLTISSLOPEDFATYYCQQLHTDPI |
| | light | chain | | TFGQGTRLEIKRTVAAPSVFIFPPSDEQLKSGTASVVCLLNNFYPREA |
| | chain | | | KVQWKVDNALQSGNSQESVTEQDSKDSTYSLSSTLTLSKADYEKHKVY |
| | sequence | | | ACEVTHQGLSSPVTKSFNRGEC |
| 277 | 413F09- | Nucleic acid | | GACATCCAGTTGACCCAGTCTCCATCCTTCCTGTCTGCATCTGTAGGA |
| | full | sequence of 413F09 | | GACAGAGTCACCATCACTTGCTGGGCCAGTCAGGACATTAGCACTTAT |
| | light | light chain | | TTAGGCTGGTATCAGCAAAAACCAGGGAAAGCCCCTAAGCTCCTGATC |
| | chain | | | TATGGTACATCCAGTTTGCAAAGTGGGGTCCCATCAAGGTTCAGCGGC |
| | sequence | | | AGTGGATCTGGGACAGAATTCACTCTCACAATCAGCAGCCTGCAGCCT |
| | | | | GAAGATTTTGCAACTTATTACTGTCAACAGCTTCATACTGACCCGATC |
| | | | | ACCTTCGGCCAAGGGACACGACTGGAGATCAAACGTACGGTGGCCGCT |
| | | | | CCCTCCGTGTTCATCTTCCCACCTTCCGACGAGCAGCTGAAGTCCGGC |
| | | | | ACCGCTTCTGTCGTGTGCCTGCTGAACAACTTCTACCCCCGCGAGGCC |
| | | | | AAGGTGCAGTGGAAGGTGGACAACGCCCTGCAGTCCGGCAACTCCCAG |
| | | | | GAATCCGTGACCGAGCAGGACTCCAAGGACAGCACCTACTCCCTGTCC |
| | | | | TCCACCCTGACCCTGTCCAAGGCCGACTACGAGAAGCACAAGGTGTAC |
| | | | | GCCTGCGAAGTGACCCACCAGGGCCTGTCTAGCCCCGTGACCAAGTCT |
| | | | | TTCAACCGGGGCGAGTGT |
| 278 | 414B06- | Amino acid sequence | | GFTFSSYW |
| | CDRH1 | of CDRH1 of 414B06 | | |
| | (IMGT) | using IMGT | | |
| 279 | 414B06- | Amino acid sequence | | IKQDGSEK |
| | CDRH2 | of CDRH2 of 414B06 | | |
| | (IMGT) | using IMGT | | |
| 280 | 414B06- | Amino acid sequence | | ARVRQWSDYSDY |
| | CDRH3 | of CDRH3 of 414B06 | | |
| | (IMGT) | using IMGT | | |
| 281 | 414B06- | Amino acid sequence | | SYWMN |
| | CDRH1 | of CDRH1 of 414B06 | | |
| | (Kabat) | using Kabat | | |
| 282 | 414B06- | Amino acid sequence | | NIKQDGSEKYYVDSVKG |
| | CDRH2 | of CDRH2 of 414B06 | | |
| | (Kabat) | using Kabat | | |
| 283 | 414B06- | Amino acid sequence | | VRQWSDYSDY |
| | CDRH3 | of CDRH3 of 414B06 | | |
| | (Kabat) | using Kabat | | |
| 284 | 414B06- | Amino acid sequence | | EVHLVESGGGLVQPGGSLRLSCAASGFTFSSYWMNWVRQAPGKGLEWV |
| | Heavy | of V_{H} of 414B06 | | ANIKQDGSEKYYVDSVKGRFTVSRDNAKNSLYLQMNSLRAEDTAVYYC |
| | chain | | | ARVRQWSDYSDYWGQGTPVTVSS |
| | variable | | | |
| | region | | | |
| 285 | 414B06- | Nucleic acid | | GAGGTGCACCTGGTGGAGTCTGGGGGAGGCTTGGTCCAGCCTGGGGGG |
| | Heavy | sequence of V_{H} of | | TCCCTGAGACTCTCCTGTGCAGCCTCTGGATTCACCTTTAGTAGCTAT |
| | chain | 414B06 | | TGGATGAACTGGGTCCGCCAGGCTCCAGGGAAGGGGCTGGAGTGGGTG |
| | variable | | | GCCAACATAAAGCAAGATGGAAGTGAGAAATACTATGTGGACTCTGTG |
| | region | | | AAGGGCCGCTTCACCGTCTCCAGAGACAACGCCAAGAACTCACTGTAT |
| | | | | CTGCAAATGAACAGCCTGAGAGCCGAGGACACGGCTGTGTATTACTGT |
| | | | | GCGAGAGTTCGACAATGGTCCGACTACTCTGACTACTGGGGCCAGGGA |
| | | | | ACCCCGGTCACCGTCTCCTCAG |
| 286 | 414B06- | Amino acid sequence | | EVHLVESGGGLVQPGGSLRLSCAASGFTFSSYWMNWVRQAPGKGLEWV |
| | full | of 414B06 heavy | | ANIKQDGSEKYYVDSVKGRFTVSRDNAKNSLYLQMNSLRAEDTAVYYC |
| | heavy | chain | | ARVROWSDYSDYWGQGTPVTVSSASTKGPSVFPLAPSSKSTSGGTAAL |
| | chain | | | GCLVKDYFPEPVTVSWNSGALTSGVHTFPAVLQSSGLYSLSSVVTVPS |
| | sequence | | | SSLGTQTYICNVNHKPSNTKVDKKVEPKSCDKTHTCPPCPAPELLGGP |
| | | | | SVFLFPPKPKDTLMISRTPEVTCVVVDVSHEDPEVKFNWYVDGVEVHN |
| | | | | AKTKPREEQYNSTYRVVSVLTVLHQDWLNGKEYKCKVSNKALPAPIEK |
| | | | | TISKAKGQPREPQVYTLPPSRDELTKNQVSLTCLVKGFYPSDIAVEWE |
| | | | | SNGQPENNYKTTPPVLDSDGSFFLYSKLTVDKSRWQQGNVFSCSVMHE |
| | | | | ALHNHYTQKSLSLSPGK |
| 287 | 414B06- | Nucleic acid | | GAGGTGCACCTGGTGGAGTCTGGGGGAGGCTTGGTCCAGCCTGGGGGG |
| | full | sequence of 414B06 | | TCCCTGAGACTCTCCTGTGCAGCCTCTGGATTCACCTTTAGTAGCTAT |
| | heavy | heavy chain | | TGGATGAACTGGGTCCGCCAGGCTCCAGGGAAGGGGCTGGAGTGGGTG |
| | chain | | | GCCAACATAAAGCAAGATGGAAGTGAGAAATACTATGTGGACTCTGTG |
| | sequence | | | AAGGGCCGCTTCACCGTCTCCAGAGACAACGCCAAGAACTCACTGTAT |
| | | | | CTGCAAATGAACAGCCTGAGAGCCGAGGACACGGCTGTGTATTACTGT |
| | | | | GCGAGAGTTCGACAATGGTCCGACTACTCTGACTACTGGGGCCAGGGA |
| | | | | ACCCCGGTCACCGTCTCCTCAGCCAGCACCAAGGGCCCCTCTGTGTTC |
| | | | | CCTCTGGCCCCTTCCAGCAAGTCCACCTCTGGCGGAACAGCCGCTCTG |
| | | | | GGCTGCCTCGTGAAGGACTACTTCCCCGAGCCTGTGACCGTGTCCTGG |
| | | | | AACTCTGGCGCTCTGACCAGCGGAGTGCACACCTTCCCTGCTGTGCTG |
| | | | | CAGTCCTCCGGCCTGTACTCCCTGTCCTCCGTCGTGACCGTGCCTTCC |
| | | | | AGCTCTCTGGGCACCCAGACCTACATCTGCAACGTGAACCACAAGCCC |
| | | | | TCCAACACCAAGGTGGACAAGAAGGTGGAACCCAAGTCCTGCGACAAG |
| | | | | ACCCACACCTGTCCCCCTTGTCCTGCCCCTGAACTGCTGGGCGGACCT |
| | | | | TCCGTGTTCCTGTTCCCCCCAAAGCCCAAGGACACCCTGATGATCTCC |
| | | | | CGGACCCCCGAAGTGACCTGCGTGGTGGTGGATGTGTCCCACGAGGAC |
| | | | | CCTGAAGTGAAGTTCAATTGGTACGTGGACGGCGTGGAAGTGCACAAC |
| | | | | GCCAAGACCAAGCCTAGAGAGGAACAGTACAACTCCACCTACCGGGTG |
| | | | | GTGTCCGTGCTGACCGTGCTGCACCAGGATTGGCTGAACGGCAAAGAG |
| | | | | TACAAGTGCAAGGTGTCCAACAAGGCCCTGCCTGCCCCCATCGAAAAG |
| | | | | ACCATCTCCAAGGCCAAGGGCCAGCCCCGGGAACCCCAGGTGTACACA |
| | | | | CTGCCCCCTAGCAGGGACGAGCTGACCAAGAACCAGGTGTCCCTGACC |
| | | | | TGTCTCGTGAAAGGCTTCTACCCCTCCGATATCGCCGTGGAATGGGAG |
| | | | | TCCAACGGCCAGCCTGAGAACAACTACAAGACCACCCCCCCTGTGCTG |
| | | | | GACTCCGACGGCTCATTCTTCCTGTACAGCAAGCTGACAGTGGACAAG |
| | | | | TCCCGGTGGCAGCAGGGCAACGTGTTCTCCTGCTCCGTGATGCACGAG |
| | | | | GCCCTGCACAACCACTACACCCAGAAGTCCCTGTCCCTGAGCCCCGGC |
| | | | | AAG |
| 288 | 414B06- | Amino acid sequence | | QGISSW |
| | CDRL1 | of CDRL1 of 414B06 | | |
| | (IMGT) | using IMGT | | |
| 289 | 414B06- | Amino acid sequence | | AAS |
| | CDRL2 | of CDRL2 of 414B06 | | |
| | (IMGT) | using IMGT | | |
| 290 | 414B06- | Amino acid sequence | | QQANSFPFT |
| | CDRL3 | of CDRL3 of 414B06 | | |
| | (IMGT) | using IMGT | | |
| 291 | 414B06- | Amino acid sequence | | RASQGISSWLA |
| | CDRL1 | of CDRL1 of 414B06 | | |
| | (Kabat) | using Kabat | | |
| 292 | 414B06- | Amino acid sequence | | AASSLOS |
| | CDRL2 | of CDRL2 of 414B06 | | |
| | (Kabat) | using Kabat | | |
| 293 | 414B06- | Amino acid sequence | | QQANSFPFT |
| | CDRL3 | of CDRL3 of 414B06 | | |
| | (Kabat) | using Kabat | | |
| 294 | 414B06- | Amino acid sequence | | DIQMTQSPSSVSASVGDRVTITCRASQGISSWLAWYQQKPGKAPKLLI |
| | Light | of V_{L} of 414B06 | | YAASSLQSGVPSRFSGSGSGTDFTLTISSLQPEDFATYYCQQANSFPF |
| | chain | | | TFGPGTKVDIK |
| | variable | | | |
| | region | | | |
| 295 | 414B06- | Nucleic acid | | GACATCCAGATGACCCAGTCTCCATCTTCCGTGTCTGCATCTGTAGGA |
| | Light | sequence of V_{L} of | | GACAGAGTCACCATCACTTGTCGGGCGAGTCAGGGTATTAGCAGCTGG |
| | chain | 414B06 | | TTAGCCTGGTATCAGCAGAAACCAGGGAAAGCCCCTAAGCTCCTGATC |
| | variable | | | TATGCTGCATCCAGTTTGCAAAGTGGGGTCCCATCAAGGTTCAGCGGC |
| | region | | | AGTGGATCTGGGACAGATTTCACTCTCACCATCAGCAGCCTGCAGCCT |
| | | | | GAAGATTTTGCAACTTACTATTGTCAACAGGCTAACAGTTTCCCATTC |
| | | | | ACTTTCGGCCCTGGGACCAAAGTGGATATCAAAC |
| 296 | 414B06- | Amino acid sequence | | DIQMTQSPSSVSASVGDRVTITCRASQGISSWLAWYQQKPGKAPKLLI |
| | full | of 414B06 light | | YAASSLQSGVPSRFSGSGSGTDFTLTISSLQPEDFATYYCQQANSFPF |
| | light | chain | | TFGPGTKVDIKRTVAAPSVFIFPPSDEQLKSGTASVVCLLNNFYPREA |
| | chain | | | KVQWKVDNALQSGNSQESVTEQDSKDSTYSLSSTLTLSKADYEKHKVY |
| | sequence | | | ACEVTHQGLSSPVTKSFNRGEC |
| 297 | 414B06- | Nucleic acid | | GACATCCAGATGACCCAGTCTCCATCTTCCGTGTCTGCATCTGTAGGA |
| | full | sequence of 414B06 | | GACAGAGTCACCATCACTTGTCGGGCGAGTCAGGGTATTAGCAGCTGG |
| | light | light chain | | TTAGCCTGGTATCAGCAGAAACCAGGGAAAGCCCCTAAGCTCCTGATC |
| | chain | | | TATGCTGCATCCAGTTTGCAAAGTGGGGTCCCATCAAGGTTCAGCGGC |
| | sequence | | | AGTGGATCTGGGACAGATTTCACTCTCACCATCAGCAGCCTGCAGCCT |
| | | | | GAAGATTTTGCAACTTACTATTGTCAACAGGCTAACAGTTTCCCATTC |
| | | | | ACTTTCGGCCCTGGGACCAAAGTGGATATCAAACGTACGGTGGCCGCT |
| | | | | CCCTCCGTGTTCATCTTCCCACCTTCCGACGAGCAGCTGAAGTCCGGC |
| | | | | ACCGCTTCTGTCGTGTGCCTGCTGAACAACTTCTACCCCCGCGAGGCC |
| | | | | AAGGTGCAGTGGAAGGTGGACAACGCCCTGCAGTCCGGCAACTCCCAG |
| | | | | GAATCCGTGACCGAGCAGGACTCCAAGGACAGCACCTACTCCCTGTCC |
| | | | | TCCACCCTGACCCTGTCCAAGGCCGACTACGAGAAGCACAAGGTGTAC |
| | | | | GCCTGCGAAGTGACCCACCAGGGCCTGTCTAGCCCCGTGACCAAGTCT |
| | | | | TTCAACCGGGGCGAGTGT |
| 298 | Mutated | Amino acid sequence | | DIQMTQSPSSLSASVGDRVTITCRASQSISSYLNWYQQKPGKAPKLLI |
| | 1D05- | of 1D05 kappa light | | YYASSLQSGVPSRFSGSGSGTDFTLTISSLQPEDFATYYCQQSYSTPI |
| | LC | chain with V to Y | | TFGQGTRLEIKRTVAAPSVFIFPPSDEQLKSGTASVVCLLNNFYPREA |
| | mutant 3 | mutation in CDRL2 | | KVQWKVDNALQSGNSQESVTEQDSKDSTYSLSSTLTLSKADYEKHKVY |
| | | highlighted | | ACEVTHQGLSSPVTKSFNRGEC |
| 299 | 1D05- | Amino acid sequence | | EVQLVESGGGLVQPGRSLRLSCAASGFTFDDYAMHWVRQVPGKGLEWV |
| | heavy | of IgG1 disabled | | SGISWIRTGIGYADSVKGRFTIFRDNAKNSLYLQMNSLRAEDTALYYC |
| | chain | variant of 1D05 | | AKDMKGSGTYGGWFDTWGQGTLVTVSSASTKGPSVFPLAPSSKSTSGG |
| | disabled | | | TAALGCLVKDYFPEPVTVSWNSGALTSGVHTFPAVLQSSGLYSLSSVV |
| | IgG1 Fc | | | TVPSSSLGTQTYICNVNHKPSNTKVDKKVEPKSCDKTHTCPPCPAPEL |
| | | | | AGAPSVFLFPPKPKDTLMISRTPEVTCVVVDVSHEDPEVKFNWYVDGV |
| | | | | EVHNAKTKPREEQYNSTYRVVSVLTVLHQDWLNGKEYKCKVSNKALPA |
| | | | | PIEKTISKAKGQPREPQVYTLPPSRDELTKNQVSLTCLVKGFYPSDIA |
| | | | | VEWESNGQPENNYKTTPPVLDSDGSFFLYSKLTVDKSRWQQGNVFSCS |
| | | | | VMHEALHNHYTQKSLSLSPGK |
| 300 | 1D05- | 1D05 Light chain | | DIQMTQSPSSLSASVGDRVTITCRASQSISSYLNWYQQKPGKAPKLLI |
| | light | sequence fused to | | YVASSLQSGVPSRFSGSGSGTDFTLTISSLQPEDFATYYCQQSYSTPI |
| | chain | wild-type human IL- | | TFGQGTRLEIKRTVAAPSVFIFPPSDEQLKSGTASVVCLLNNFYPREA |
| | IL-2 | 2 sequence (IL-2 | | KVQWKVDNALQSGNSQESVTEQDSKDSTYSLSSTLTLSKADYEKHKVY |
| | fusion | amino acid sequence | | ACEVTHQGLSSPVTKSFNRGECAPTSSSTKKTQLQLEHLLLDLQMILN |
| | | is underlined and | | GINNYKNPKLTRMLTFKFYMPKKATELKHLQCLEEELKPLEEVLNLAQ |
| | | region to be varied | | SKNFHLRPRDLISNINVIVLELKGSETTEMCEYADETATIVEFLNRWI |
| | | is shown in bold) | | TFCQSIISTLT |
| 301 | Human | Uniprot number: | | APTSSSTKKTQLQLEHLLLDLQMILNGINNYKNPKLTRMLTFKFYMPK |
| | IL-2 | P60568 | | KATELKHLQCLEEELKPLEEVLNLAQSKNFHLRPRDLISNINVIVLEL |
| | | Full length amino | | KGSETTFMCEYADETATIVEFLNRWITFCQSIISTLT |
| | | acid sequence of | | |
| | | human IL-2 (minus | | |
| | | signal sequence) | | |
| 302 | Control | Heavy chain 1D05 | | EVQLVESGGGLVQPGRSLRLSCAASGFTFDDYAMHWVRQVPGKGLEWV |
| | 1D05 | IgG1 variant fused | | SGISWIRTGIGYADSVKGRFTIFRDNAKNSLYLQMNSLRAEDTALYYC |
| | immuno- | at the N-terminus | | AKDMKGSGTYGGWFDTWGQGTLVTVSSASTKGPSVFPLAPSSKSTSGG |
| | cytokine | to wild-type human | | TAALGCLVKDYFPEPVTVSWNSGALTSGVHTFPAVLQSSGLYSLSSVV |
| | HC C- | IL2 sequence | | TVPSSSLGTQTYICNVNHKPSNTKVDKKVEPKSCDKTHTCPPCPAPEL |
| | terminal | (control) | | AGAPSVFLFPPKPKDTLMISRTPEVTCVVVDVSHEDPEVKFNWYVDGV |
| | fusion | | | EVHNAKTKPREEQYNSTYRVVSVLTVLHQDWLNGKEYKCKVSNKALPA |
| | | | | PIEKTISKAKGQPREPQVYTLPPSRDELTKNQVSLTCLVKGFYPSDIA |
| | | | | VEWESNGQPENNYKTTPPVLDSDGSFFLYSKLTVDKSRWQQGNVFSCS |
| | | | | VMHEALHNHYTQKSLSLSPGKAPTSSSTKKTQLQLEHLLLDLQMILNG |
| | | | | INNYKNPKLTRMLTFKFYMPKKATELKHLQCLEEELKPLEEVLNLAQS |
| | | | | KNFHLRPRDLISNINVIVLELKGSETTEMCEYADETATIVEFLNRWIT |
| | | | | FCQSIISTLT |
| 303 | IL-2 D5- | IL-2 IC45 (Del 5-9) | | APTSTQLQLELLLD |
| | 9 | N terminal IL-2 | | |
| | | sequence | | |
| 304 | IL-2 D1- | IL-2 IC46 (Del 1- | | TQLQLEHLLLD |
| | 9 | 9) N terminal IL-2 | | |
| | | sequence | | |
| 305 | IL-2 D5- | IL-2 IC64 (Del 5-7) | | APTSKKTQLQLEHLLLD |
| | 7 | N terminal IL-2 | | |
| | | sequence | | |
| 306 | IL-2 D1 | IL-2 D1 N terminal | | PTSSSTKKTQLQLEHLLLD |
| | | IL-2 sequence | | |
| 307 | IL-2 D1- | IL-2 D1-2 N | | TSSSTKKTQLQLEHLLLD |
| | 2 | terminal IL-2 | | |
| | | sequence | | |
| 308 | IL-2 D1- | IL-2 D1-3 N | | SSSTKKTQLQLEHLLLD |
| | 3 | terminal IL-2 | | |
| | | sequence | | |
| 309 | IL-2 D1- | IL-2 D1-4 N | | SSTKKTQLQLEHLLLD |
| | 4 | terminal IL-2 | | |
| | | sequence | | |
| 310 | IL-2 D1- | IL-2 D1-5 N | | STKKTQLQLEHLLLD |
| | 5 | terminal IL-2 | | |
| | | sequence | | |
| 311 | IL-2 D1- | IL-2 D1-6 N | | TKKTQLQLEHLLLD |
| | 6 | terminal IL-2 | | |
| | | sequence | | |
| 312 | IL-2 D1- | IL-2 D1-7 N | | KKTQLQLEHLLLD |
| | 7 | terminal IL-2 | | |
| | | sequence | | |
| 313 | IL-2 D1- | IL-2 D1-8 N | | KTQLQLEHLLLD |
| | 8 | terminal IL-2 | | |
| | | sequence | | |
| 314 | IL-2 D9 | IL-2 D9 N terminal | | APTSSSTKTQLQLEHLLLD |
| | | IL-2 sequence | | |
| 315 | IL-2 D9- | IL-2 D9-8 N | | APTSSSTTQLQLEHLLLD |
| | 8 | terminal IL-2 | | |
| | | sequence | | |
| 316 | IL-2 D9- | IL-2 D9-7 N | | APTSSSTQLQLEHLLLD |
| | 7 | terminal IL-2 | | |
| | | sequence | | |
| 317 | IL-2 D9- | IL-2 D9-6 N | | APTSSTQLQLEHLLLD |
| | 6 | terminal IL-2 | | |
| | | sequence | | |
| 318 | IL-2 D9- | IL-2 D9-4 N | | APTTQLQLEHLLLD |
| | 4 | terminal IL-2 | | |
| | | sequence | | |
| 319 | IL-2 D9- | IL-2 D9-3 N | | APTQLQLEHLLLD |
| | 3 | terminal IL-2 | | |
| | | sequence | | |
| 320 | IL-2 D9- | IL-2 D9-2 N | | ATQLQLEHLLLD |
| | 2 | terminal IL-2 | | |
| | | sequence | | |
| 321 | IL-2 D2- | IL-2 D2-6 N | | ATKKTQLQLEHLLLD |
| | 6 | terminal IL-2 | | |
| | | sequence | | |
| 322 | IL-2 D3- | IL-2 D3-7 N | | APKKTQLQLEHLLLD |
| | 7 | terminal IL-2 | | |
| | | sequence | | |
| 323 | IL-2 D4- | IL-2 D4-8 N | | APTKTQLQLEHLLLD |
| | 8 | terminal IL-2 | | |
| | | sequence | | |
| 324 | C- | Amino acids 21 to | | LQMILNGINNYKNPKLTRMLTFKFYMPKKATELKHLQCLEEELKPLEE |
| | terminal | 133 of hIL-2 | | VLNLAQSKNFHLRPRDLISNINVIVLELKGSETTFMCEYADETATIVE |
| | amino | | | FLNRWITFCQSIISTLT |
| | acid | | | |
| | sequence | | | |
| | of hIL-2 | | | |
| 325 | Mouse | Uniprot number: | | MRIFAGIIFTACCHLLRAFTITAPKDLYVVEYGSNVTMECRFPVEREL |
| | PD-L1 | Q9EP73 | | DLLALVVYWEKEDEQVIQFVAGEEDLKPQHSNFRGRASLPKDQLLKGN |
| | | (ECD highlighted in | | AALQITDVKLQDAGVYCCIISYGGADYKRITLKVNAPYRKINQRISVD |
| | | BOLD, and | | PATSEHELICQAEGYPEAEVIWTNSDHQPVSGKRSVTTSRTEGMLLNV |
| | | cytoplasmic domain | | TSSLRVNATANDVFYCTFWRSQPGQNHTAELIIPELPATHPPQNRTHW |
| | | underlined) | | VLLGSILLFLIVVSTVLLFLRKQVRMLDVEKCGVEDTSSKNRNDTQFE |
| | | | | ET |
| 326 | Mouse | Mouse PD-L1 | | FTITAPKDLYVVEYGSNVTMECRFPVERELDLLALVVYWEKEDEQVIQ |
| | PD-L1 | extracellular | | FVAGEEDLKPQHSNFRGRASLPKDQLLKGNAALQITDVKLQDAGVYCC |
| | ECD His | domain with his tag | | IISYGGADYKRITLKVNAPYRKINQRISVDPATSEHELICQAEGYPEA |
| | | | | EVIWTNSDHQPVSGKRSVTTSRTEGMLLNVTSSLRVNATANDVFYCTF |
| | | | | WRSQPGQNHTAELIIPELPATHPPQNRTHHHHHH |
| 327 | Human | Human IL-2 receptor | | ELCDDDPPEIPHATFKAMAYKEGTMLNCECKRGFRRIKSGSLYMLCTG |
| | IL-2Rα | alpha chain | | NSSHSSWDNQCQCTSSATRNTTKQVTPQPEEQKERKTTEMQSPMQPVD |
| | chain | | | QASLPGHCREPPPWENEATERIYHFVVGQMVYYQCVQGYRALHRGPAE |
| | | | | SVCKMTHGKTRWTQPQLICTGEMETSQFPGEEKPQASPEGRPESETSC |
| | | | | LVTTTDFQIQTEMAATMETSIFTTEYQVAVAGCVELLISVLLLSGLTW |
| | | | | QRRQRKSRRTI |
| 328 | Human | Human IL-2 receptor | | AVNGTSQFTCFYNSRANISCVWSQDGALQDTSCQVHAWPDRRRWNQTC |
| | IL-2Rβ | beta chain | | ELLPVSQASWACNLILGAPDSQKLTTVDIVTLRVLCREGVRWRVMAIQ |
| | chain | | | DFKPFENLRLMAPISLQVVHVETHRCNISWEISQASHYFERHLEFEAR |
| | | | | TLSPGHTWEEAPLLTLKQKQEWICLETLTPDTQYEFQVRVKPLQGEFT |
| | | | | TWSPWSQPLAFRTKPAALGKDTIPWLGHLLVGLSGAFGFIILVYLLIN |
| | | | | CRNTGPWLKKVLKCNTPDPSKFFSQLSSEHGGDVQKWLSSPFPSSSES |
| | | | | PGGLAPEISPLEVLERDKVTQLLLQQDKVPEPASLSSNHSLTSCFTNQ |
| | | | | GYFFFHLPDALEIEACQVYFTYDPYSEEDPDEGVAGAPTGSSPQPLQP |
| | | | | LSGEDDAYCTFPSRDDLLLFSPSLLGGPSPPSTAPGGSGAGEERMPPS |
| | | | | LQERVPRDWDPQPLGPPTPGVPDLVDFQPPPELVLREAGEEVPDAGPR |
| | | | | EGVSFPWSRPPGQGEFRALNARLPLNTDAYLSLQELQGQDPTHLV |
| 329 | Human | Human IL-2 receptor | | LNTTILTPNGNEDTTADFFLTTMPTDSLSVSTLPLPEVQCFVENVEYM |
| | IL-2Rγ | common gamma chain | | NCTWNSSSEPQPTNLTLHYWYKNSDNDKVQKCSHYLFSEEITSGCQLQ |
| | chain | | | KKEIHLYQTFVVQLQDPREPRRQATQMLKLQNLVIPWAPENLTLHKLS |
| | | | | ESQLELNWNNRFLNHCLEHLVQYRTDWDHSWTEQSVDYRHKFSLPSVD |
| | | | | GQKRYTFRVRSRFNPLCGSAQHWSEWSHPIHWGSNTSKENPFLFALEA |
| | | | | VVISVGSMGLIISLLCVYFWLERTMPRIPTLKNLEDLVTEYHGNESAW |
| | | | | SGVSKGLAESLQPDYSERLCLVSEIPPKGGALGEGPGASPCNQHSPYW |
| | | | | APPCYTLKPET |
| 330 | IL-7 | Human IL-7 amino | | DCDIEGKDGKQYESVLMVSIDQLLDSMKEIGSNCLNNEFNFFKRHICD |
| | | acid sequence | | ANKEGMFLFRAARKLRQFLKMNSTGDFDLHLLKVSEGTTILLNCTGQV |
| | | | | KGRKPAALGEAQPTKSLEENKSLKEQKKLNDLCFLKRLLQEIKTCWNK |
| | | | | ILMGTKEH |
| 331 | IL-15 | Human IL-15 amino | | GIHVFILGCFSAGLPKTEANWVNVISDLKKIEDLIQSMHIDATLYTES |
| | | acid sequence | | DVHPSCKVTAMKCFLLELQVISLESGDASIHDTVENLIILANNSLSSN |
| | | | | GNVTESGCKECEELEEKNIKEFLQSFVHIVQMFINTS |
| 332 | IL-21 | Human IL-21 amino | | QGQDRHMIRMRQLIDIVDQLKNYVNDLVPEFLPAPEDVETNCEWSAFS |
| | | acid sequence | | CFQKAQLKSANTGNNERIINVSIKKLKRKPPSTNAGRRQKHRLTCPSC |
| | | | | DSYEKKPPKEFLERFKSLLQKMIHQHLSSRTHGSEDS |
| 333 | GM-CSF | Human GM-CSF amino | | APARSPSPSTQPWEHVNAIQEARRLLNLSRDTAAEMNETVEVISEMED |
| | | acid sequence | | LQEPTCLQTRLELYKQGLRGSLTKLKGPLTMMASHYKQHCPPTPETSC |
| | | | | ATQIITFESFKENLKDELLVIPFDCWEPVQE |
| 334 | IFNα | Human IFN-α amino | | CDLPQNHGLLSRNTLVLLHQMRRISPFLCLKDRRDFRFPQEMVKGSQL |
| | | acid sequence | | QKAHVMSVLHEMLQQIFSLFHTERSSAAWNMTLLDQLHTELHQQLQHL |
| | | | | ETCLLQVVGEGESAGAISSPALTLRRYFQGIRVYLKEKKYSDCAWEVV |
| | | | | RMEIMKSLFLSTNMQERLRSKDRDLGS |
| 335 | TNFα | Extracellular | | GPQREEFPRDLSLISPLAQAVRSSSRTPSDKPVAHVVANPQAEGQLQW |
| | | portion of human | | LNRRANALLANGVELRDNQLVVPSEGLYLIYSQVLFKGQGCPSTHVLL |
| | | TNF-α amino acid | | THTISRIAVSYQTKVNLLSAIKSPCQRETPEGAEAKPWYEPIYLGGVF |
| | | sequence | | QLEKGDRLSAEINRPDYLDFAESGQVYFGIIAL |
| 336 | IL-12α | Alpha chain of | | RNLPVATPDPGMFPCLHHSQNLLRAVSNMLQKARQTLEFYPCTSEEID |
| | | human IL-12 amino | | HEDITKDKTSTVEACLPLELTKNESCLNSRETSFITNGSCLASRKTSF |
| | | acid sequence | | MMALCLSSIYEDLKMYQVEFKTMNAKLLMDPKRQIFLDQNMLAVIDEL |
| | | | | MQALNFNSETVPQKSSLEEPDFYKTKIKLCILLHAFRIRAVTIDRVMS |
| | | | | YLNAS |
| 337 | IL-12ß | Beta chain of human | | IWELKKDVYVVELDWYPDAPGEMVVLTCDTPEEDGITWTLDQSSEVLG |
| | | IL-12 amino acid | | SGKTLTIQVKEFGDAGQYTCHKGGEVLSHSLLLLHKKEDGIWSTDILK |
| | | sequence | | DQKEPKNKTFLRCEAKNYSGRFTCWWLTTISTDLTFSVKSSRGSSDPQ |
| | | | | GVTCGAATLSAERVRGDNKEYEYSVECQEDSACPAAEESLPIEVMVDA |
| | | | | VHKLKYENYTSSFFIRDIIKPDPPKNLQLKPLKNSRQVEVSWEYPDTW |
| | | | | STPHSYFSLTFCVQVQGKSKREKKDRVFTDKTSATVICRKNASISVRA |
| | | | | QDRYYSSSWSEWASVPCS |
| 338 | CXCL9 | Human CXCL-9 amino | | TPVVRKGRCSCISTNQGTIHLQSLKDLKQFAPSPSCEKIEIIATLKNG |
| | | acid sequence | | VQTCLNPDSADVKELIKKWEKQVSQKKKQKNGKKHQKKKVLKVRKSQR |
| | | | | SRQKKTT |
| 339 | CXCL10 | Human CXCL-10 amino | | VPLSRTVRCTCISISNQPVNPRSLEKLEIIPASQFCPRVEIIATMKKK |
| | | acid sequence | | GEKRCLNPESKAIKNLLKAVSKERSKRSP |
| 340 | Human WT | IGHG1*01 | WT human | ASTKGPSVFPLAPSSKSTSGGTAALGCLVKDYFPEPVTVSWNSGALTS |
| | IgG1 | & | IgG1 amino | GVHTFPAVLQSSGLYSLSSVVTVPSSSLGTQTYICNVNHKPSNTKVDK |
| | constant | IGHG1*02 | acid | KVEPKSCDKTHTCPPCPAPELLGGPSVFLFPPKPKDTLMISRTPEVTC |
| | region | & | sequence | VVVDVSHEDPEVKFNWYVDGVEVHNAKTKPREEQYNSTYRVVSVLTVL |
| | | IGHG1*05 | | HQDWLNGKEYKCKVSNKALPAPIEKTISKAKGQPREPQVYTLPPSRDE |
| | | (IgG1) | | LTKNQVSLTCLVKGFYPSDIAVEWESNGQPENNYKTTPPVLDSDGSFF |
| | | | | LYSKLTVDKSRWQQGNVFSCSVMHEALHNHYTQKSLSLSPGK |
| 341 | | | WT human | GCCAGCACCAAGGGCCCCTCTGTGTTCCCTCTGGCCCCTTCCAGCAAG |
| | | | IgG1 | TCCACCTCTGGCGGAACAGCCGCTCTGGGCTGCCTCGTGAAGGACTAC |
| | | | nucleic | TTCCCCGAGCCTGTGACCGTGTCCTGGAACTCTGGCGCTCTGACCAGC |
| | | | acid | GGAGTGCACACCTTCCCTGCTGTGCTGCAGTCCTCCGGCCTGTACTCC |
| | | | sequence | CTGTCCTCCGTCGTGACCGTGCCTTCCAGCTCTCTGGGCACCCAGACC |
| | | | | TACATCTGCAACGTGAACCACAAGCCCTCCAACACCAAGGTGGACAAG |
| | | | | AAGGTGGAACCCAAGTCCTGCGACAAGACCCACACCTGTCCCCCTTGT |
| | | | | CCTGCCCCTGAACTGCTGGGCGGACCTTCCGTGTTCCTGTTCCCCCCA |
| | | | | AAGCCCAAGGACACCCTGATGATCTCCCGGACCCCCGAAGTGACCTGC |
| | | | | GTGGTGGTGGATGTGTCCCACGAGGACCCTGAAGTGAAGTTCAATTGG |
| | | | | TACGTGGACGGCGTGGAAGTGCACAACGCCAAGACCAAGCCTAGAGAG |
| | | | | GAACAGTACAACTCCACCTACCGGGTGGTGTCCGTGCTGACCGTGCTG |
| | | | | CACCAGGATTGGCTGAACGGCAAAGAGTACAAGTGCAAGGTGTCCAAC |
| | | | | AAGGCCCTGCCTGCCCCCATCGAAAAGACCATCTCCAAGGCCAAGGGC |
| | | | | CAGCCCCGGGAACCCCAGGTGTACACACTGCCCCCTAGCAGGGACGAG |
| | | | | CTGACCAAGAACCAGGTGTCCCTGACCTGTCTCGTGAAAGGCTTCTAC |
| | | | | CCCTCCGATATCGCCGTGGAATGGGAGTCCAACGGCCAGCCTGAGAAC |
| | | | | AACTACAAGACCACCCCCCCTGTGCTGGACTCCGACGGCTCATTCTTC |
| | | | | CTGTACAGCAAGCTGACAGTGGACAAGTCCCGGTGGCAGCAGGGCAAC |
| | | | | GTGTTCTCCTGCTCCGTGATGCACGAGGCCCTGCACAACCACTACACC |
| | | | | CAGAAGTCCCTGTCCCTGAGCCCCGGCAAGTGATGA |

| | | | |
|---|---|---|---|
| 342 | Mutated | Amino acid sequence | EVQLVESGGGLVQPGRSLRLSCAASGFTFDDYAMHWVRQAPGKGLEWV |
| | 1D05 | of 1D05 heavy chain | SGISWIRTGIGYADSVKGRFTISRDNAKNSLYLQMNSLRAEDTALYYC |
| | HC | with V to A and F | AKDMKGSGTYGGWFDTWGQGTLVTVSSASTKGPSVFPLAPCSRSTSES |
| | mutant 2 | to S back-mutation | TAALGCLVKDYFPEPVTVSWNSGALTSGVHTFPAVLQSSGLYSLSSVV |
| | | in framework region | TVPSSSLGTKTYTCNVDHKPSNTKVDKRVESKYGPPCPPCPAPELAGA |
| | | to germline | PSVFLFPPKPKDTLMISRTPEVTCVVVDVSQEDPEVQFNWYVDGVEVH |
| | | highlighted with | NAKTKPREEQFNSTYRVVSVLTVLHQDWLNGKEYKCKVSNKGLPSSIE |
| | | IgG1 disabled | KTISKAKGQPREPQVYTLPPSQEEMTKNQVSLTCLVKGFYPSDIAVEW |
| | | (LAGA) constant | ESNGQPENNYKTTPPVLDSDGSFFLYSRLTVDKSRWQEGNVFSCSVMH |
| | | region | EALHNHYTQKSLSLSLGK |

TABLE S2
SEQ ID NOS: 343-538

| SEQ | | | |
|---|---|---|---|
| NO: | Name | Description | Sequence |
| 343 | 416E01- | Amino acid sequence | GFTFSNYA |
| | CDRH1 | of CDRH1 of 416E01 | |
| | (IMGT) | using IMGT | |
| 344 | 416E01 | Amino acid sequence | ISFSGGTT |
| | CDRH2 | of CDRH2 of 416E01 | |
| | (IMGT) | using IMGT | |
| 345 | 416E01 | Amino acid sequence | AKDEAPAGATFFDS |
| | CDRH3 | of CDRH3 of 416E01 | |
| | (IMGT) | using IMGT | |
| 346 | 416E01- | Amino acid sequence | NYAMS |
| | CDRH1 | of CDRH1 of 416E01 | |
| | (Kabat) | using Kabat | |
| 347 | 416E01 | Amino acid sequence | AISFSGGTTYYADSVKG |
| | CDRH2 | of CDRH2 of 416E01 | |
| | (Kabat) | using Kabat | |
| 348 | 416E01- | Amino acid sequence | DEAPAGATFFDS |
| | CDRH3 | of CDRH3 of 416E01 | |
| | (Kabat) | using Kabat | |
| 349 | 416E01 | Amino acid sequence | EVQLAESGGGLVQPGGSLRLSCAASGFTFSNYAMSWVRQTPGKGLE |
| | Heavy | of V_{H} of 416E01 | WVSAISFSGGTTYYADSVKGRFTISRDNSKNTLYLHMNSLRADDTA |
| | chain | (mutations from | VYYCAKDEAPAGATFFDSWGQGTLVTVSS |
| | variable | germline are shown | |
| | region | in bold letters) | |
| 350 | 416E01 | Nucleic acid | GAAGTGCAACTGGCGGAGTCTGGGGGAGGCTTGGTACAGCCGGGGG |
| | Heavy | sequence of V_{H} of | GGTCCCTGAGACTCTCCTGTGCAGCCTCTGGATTCACCTTTAGCAA |
| | chain | 416E01 | CTATGCCATGAGTTGGGTCCGCCAGACTCCAGGAAAGGGGCTGGAG |
| | variable | | TGGGTCTCAGCTATTAGTTTTAGTGGTGGTACTACATACTACGCTG |
| | region | | ACTCCGTGAAGGGCCGGTTCACCATCTCCAGAGACAATTCCAAGAA |
| | | | CACGCTGTATTTGCACATGAACAGCCTGAGAGCCGATGACACGGCC |
| | | | GTATATTACTGTGCGAAAGATGAGGCACCAGCTGGCGCAACCTTCT |
| | | | TTGACTCCTGGGGCCAGGGAACGCTGGTCACCGTCTCCTCAG |
| 351 | 416E01- | Amino acid sequence | EVQLAESGGGLVQPGGSLRLSCAASGFTFSNYAMSWVRQTPGKGLE |
| | full | of 416E01 heavy | WVSAISFSGGTTYYADSVKGRFTISRDNSKNTLYLHMNSLRADDTA |
| | heavy | chain | VYYCAKDEAPAGATFFDSWGQGTLVTVSSASTKGPSVFPLAPCSRS |
| | chain | | TSESTAALGCLVKDYFPEPVTVSWNSGALTSGVHTFPAVLQSSGLY |
| | sequence | | SLSSVVTVPSSSLGTKTYTCNVDHKPSNTKVDKRVESKYGPPCPPC |
| | | | PAPEFEGGPSVFLFPPKPKDTLMISRTPEVTCVVVDVSQEDPEVQF |
| | | | NWYVDGVEVHNAKTKPREEQFNSTYRVVSVLTVLHQDWLNGKEYKC |
| | | | KVSNKGLPSSIEKTISKAKGQPREPQVYTLPPSQEEMTKNQVSLTC |
| | | | LVKGFYPSDIAVEWESNGQPENNYKTTPPVLDSDGSFFLYSRLTVD |
| | | | KSRWQEGNVFSCSVMHEALHNHYTQKSLSLSLGK |
| 352 | 416E01- | Nucleic acid | GAAGTGCAACTGGCGGAGTCTGGGGGAGGCTTGGTACAGCCGGGGG |
| | full | sequence of 416E01 | GGTCCCTGAGACTCTCCTGTGCAGCCTCTGGATTCACCTTTAGCAA |
| | heavy | heavy chain | CTATGCCATGAGTTGGGTCCGCCAGACTCCAGGAAAGGGGCTGGAG |
| | chain | | TGGGTCTCAGCTATTAGTTTTAGTGGTGGTACTACATACTACGCTG |
| | sequence | | ACTCCGTGAAGGGCCGGTTCACCATCTCCAGAGACAATTCCAAGAA |
| | | | CACGCTGTATTTGCACATGAACAGCCTGAGAGCCGATGACACGGCC |
| | | | GTATATTACTGTGCGAAAGATGAGGCACCAGCTGGCGCAACCTTCT |
| | | | TTGACTCCTGGGGCCAGGGAACGCTGGTCACCGTCTCCTCAGCCAG |
| | | | CACCAAGGGCCCTTCCGTGTTCCCCCTGGCCCCTTGCAGCAGGAGC |
| | | | ACCTCCGAATCCACAGCTGCCCTGGGCTGTCTGGTGAAGGACTACT |
| | | | TTCCCGAGCCCGTGACCGTGAGCTGGAACAGCGGCGCTCTGACATC |
| | | | CGGCGTCCACACCTTTCCTGCCGTCCTGCAGTCCTCCGGCCTCTAC |
| | | | TCCCTGTCCTCCGTGGTGACCGTGCCTAGCTCCTCCCTCGGCACCA |
| | | | AGACCTACACCTGTAACGTGGACCACAAACCCTCCAACACCAAGGT |
| | | | GGACAAACGGGTCGAGAGCAAGTACGGCCCTCCCTGCCCTCCTTGT |
| | | | CCTGCCCCCGAGTTCGAAGGCGGACCCAGCGTGTTCCTGTTCCCTC |
| | | | CTAAGCCCAAGGACACCCTCATGATCAGCCGGACACCCGAGGTGAC |
| | | | CTGCGTGGTGGTGGATGTGAGCCAGGAGGACCCTGAGGTCCAGTTC |
| | | | AACTGGTATGTGGATGGCGTGGAGGTGCACAACGCCAAGACAAAGC |
| | | | CCCGGGAAGAGCAGTTCAACTCCACCTACAGGGTGGTCAGCGTGCT |
| | | | GACCGTGCTGCATCAGGACTGGCTGAACGGCAAGGAGTACAAGTGC |
| | | | AAGGTCAGCAATAAGGGACTGCCCAGCAGCATCGAGAAGACCATCT |
| | | | CCAAGGCTAAAGGCCAGCCCCGGGAACCTCAGGTGTACACCCTGCC |
| | | | TCCCAGCCAGGAGGAGATGACCAAGAACCAGGTGAGCCTGACCTGC |
| | | | CTGGTGAAGGGATTCTACCCTTCCGACATCGCCGTGGAGTGGGAGT |
| | | | CCAACGGCCAGCCCGAGAACAATTATAAGACCACCCCTCCCGTCCT |
| | | | CGACAGCGACGGATCCTTCTTTCTGTACTCCAGGCTGACCGTGGAT |
| | | | AAGTCCAGGTGGCAGGAAGGCAACGTGTTCAGCTGCTCCGTGATGC |
| | | | ACGAGGCCCTGCACAATCACTACACCCAGAAGTCCCTGAGCCTGTC |
| | | | CCTGGGAAAG |
| 353 | 416E01- | Amino acid sequence | QGIRRW |
| | CDRL1 | of CDRL1 of 416E01 | |
| | (IMGT) | using IMGT | |
| 354 | 416E01- | Amino acid sequence | GAS |
| | CDRL2 | of CDRL2 of 416E01 | |
| | (IMGT) | using IMGT | |
| 355 | 416E01- | Amino acid sequence | QQANSFPIT |
| | CDRL3 | of CDRL3 of 416E01 | |
| | (IMGT) | using IMGT | |
| 356 | 416E01- | Amino acid sequence | RASQGIRRWLA |
| | CDRL1 | of CDRL1 of 416E01 | |
| | (Kabat) | using Kabat | |
| 357 | 416E01- | Amino acid sequence | GASSLQS |
| | CDRL2 | of CDRL2 of 416E01 | |
| | (Kabat) | using Kabat | |
| 358 | 416E01- | Amino acid sequence | QQANSFPIT |
| | CDRL3 | of CDRL3 of 416E01 | |
| | (Kabat) | using Kabat | |
| 359 | 416E01 | Amino acid sequence | DIQMTQSPSSVSASVGDRVTITCRASQGIRRWLAWYQQKPGKAPKL |
| | Light | of V_{L} of | LISGASSLQSGVPSRFSGSGSGTDFTLIITSLQPEDFATYYCQQAN |
| | chain | 416E01 (mutations | SFPITFGQGTRLEIK |
| | variable | from germline are | |
| | region | shown in bold | |
| | | letters) | |
| 360 | 416E01- | Nucleic acid | GACATCCAGATGACCCAGTCTCCATCTTCCGTGTCTGCATCTGTAG |
| | Light | sequence of V_{L} of | GAGACAGAGTCACCATCACTTGTCGGGCGAGTCAGGGTATTAGGAG |
| | chain | 416E01 | GTGGTTAGCCTGGTATCAGCAGAAACCAGGGAAAGCCCCTAAACTC |
| | variable | | CTGATCTCTGGTGCATCCAGTTTGCAAAGTGGGGTCCCATCAAGGT |
| | region | | TCAGCGGCAGTGGATCTGGGACAGATTTCACTCTCATCATTACCAG |
| | | | TCTGCAGCCTGAAGATTTTGCAACTTACTATTGTCAACAGGCTAAC |
| | | | AGTTTCCCGATCACCTTCGGCCAAGGGACACGACTGGAGATCAAAC |
| 361 | 416E01- | Amino acid sequence | DIQMTQSPSSVSASVGDRVTITCRASQGIRRWLAWYQQKPGKAPKL |
| | full | of 416E01 light | LISGASSLQSGVPSRFSGSGSGTDFTLIITSLQPEDFATYYCQQAN |
| | light | chain | SFPITFGQGTRLEIKRTVAAPSVFIFPPSDEQLKSGTASVVCLLNN |
| | chain | | FYPREAKVQWKVDNALQSGNSQESVTEQDSKDSTYSLSSTLTLSKA |
| | sequence | | DYEKHKVYACEVTHQGLSSPVTKSENRGEC |
| 362 | 416E01- | Nucleic acid | GACATCCAGATGACCCAGTCTCCATCTTCCGTGTCTGCATCTGTAG |
| | full | sequence of 416E01 | GAGACAGAGTCACCATCACTTGTCGGGCGAGTCAGGGTATTAGGAG |
| | light | light chain | GTGGTTAGCCTGGTATCAGCAGAAACCAGGGAAAGCCCCTAAACTC |
| | chain | | CTGATCTCTGGTGCATCCAGTTTGCAAAGTGGGGTCCCATCAAGGT |
| | sequence | | TCAGCGGCAGTGGATCTGGGACAGATTTCACTCTCATCATTACCAG |
| | | | TCTGCAGCCTGAAGATTTTGCAACTTACTATTGTCAACAGGCTAAC |
| | | | AGTTTCCCGATCACCTTCGGCCAAGGGACACGACTGGAGATCAAAC |
| | | | GTACGGTGGCCGCTCCCTCCGTGTTCATCTTCCCACCTTCCGACGA |
| | | | GCAGCTGAAGTCCGGCACCGCTTCTGTCGTGTGCCTGCTGAACAAC |
| | | | TTCTACCCCCGCGAGGCCAAGGTGCAGTGGAAGGTGGACAACGCCC |
| | | | TGCAGTCCGGCAACTCCCAGGAATCCGTGACCGAGCAGGACTCCAA |
| | | | GGACAGCACCTACTCCCTGTCCTCCACCCTGACCCTGTCCAAGGCC |
| | | | GACTACGAGAAGCACAAGGTGTACGCCTGCGAAGTGACCCACCAGG |
| | | | GCCTGTCTAGCCCCGTGACCAAGTCTTTCAACCGGGGCGAGTGT |
| 363 | STIM001 | Amino acid sequence | GYTFSTFG |
| | -CDRH1 | of CDRH1 of STIM001 | |
| | | using IMGT | |
| 364 | STIM001 | Amino acid sequence | ISAYNGDT |
| | -CDRH2 | of CDRH2 of STIM001 | |
| | | using IMGT | |
| 365 | STIM001 | Amino acid sequence | ARSSGHYYYYGMDV |
| | -CDRH3 | of CDRH3 of STIM001 | |
| | | using IMGT | |
| 366 | STIM001 | Amino acid sequence | QVQVVQSGAEVKKPGASVKVSCKASGYTFSTFGITWVRQAPGQGLE |
| | -Heavy | of V_{H} of STIM001 | WMGWISAYNGDTNYAQNLQGRVIMTTDTSTSTAYMELRSLRSDDTA |
| | chain | | VYYCARSSGHYYYYGMDVWGQGTTVTVSS |
| | variable | | |
| | region | | |
| 367 | STIM001 | Nucleic acid | CAGGTTCAGGTGGTGCAGTCTGGAGCTGAGGTGAAGAAGCCTGGGG |
| | -Heavy | sequence of V_{H} of | CCTCAGTGAAGGTCTCCTGCAAGGCTTCTGGTTACACCTTTTCCAC |
| | chain | STIM001 | CTTTGGTATCACCTGGGTGCGACAGGCCCCTGGACAAGGGCTTGAA |
| | variable | | TGGATGGGATGGATCAGCGCTTACAATGGTGACACAAACTATGCAC |
| | region | | AGAATCTCCAGGGCAGAGTCATCATGACCACAGACACATCCACGAG |
| | | | CACAGCCTACATGGAGCTGAGGAGCCTGAGATCTGACGACACGGCC |
| | | | GTTTATTACTGTGCGAGGAGCAGTGGCCACTACTACTACTACGGTA |
| | | | TGGACGTCTGGGGCCAAGGGACCACGGTCACCGTCTCCTCA |
| 368 | STIM001 | Amino acid sequence | QVQVVQSGAEVKKPGASVKVSCKASGYTFSTFGITWVRQAPGQGLE |
| | -full | of STIM001 heavy | WMGWISAYNGDTNYAQNLQGRVIMTTDTSTSTAYMELRSLRSDDTA |
| | heavy | chain | VYYCARSSGHYYYYGMDVWGQGTTVTVSSASTKGPSVFPLAPSSKS |
| | chain | | TSGGTAALGCLVKDYFPEPVTVSWNSGALTSGVHTFPAVLQSSGLY |
| | sequence | | SLSSVVTVPSSSLGTQTYICNVNHKPSNTKVDKKVEPKSCDKTHTC |
| | | | PPCPAPELLGGPSVFLFPPKPKDTLMISRTPEVTCVVVDVSHEDPE |
| | | | VKFNWYVDGVEVHNAKTKPREEQYNSTYRVVSVLTVLHQDWLNGKE |
| | | | YKCKVSNKALPAPIEKTISKAKGQPREPQVYTLPPSRDELTKNQVS |
| | | | LTCLVKGFYPSDIAVEWESNGQPENNYKTTPPVLDSDGSFFLYSKL |
| | | | TVDKSRWQQGNVFSCSVMHEALHNHYTQKSLSLSPGK |
| 369 | STIM001 | Nucleic acid | CAGGTTCAGGTGGTGCAGTCTGGAGCTGAGGTGAAGAAGCCTGGGG |
| | -full | sequence of STIM001 | CCTCAGTGAAGGTCTCCTGCAAGGCTTCTGGTTACACCTTTTCCAC |
| | heavy | heavy chain | CTTTGGTATCACCTGGGTGCGACAGGCCCCTGGACAAGGGCTTGAA |
| | chain | | TGGATGGGATGGATCAGCGCTTACAATGGTGACACAAACTATGCAC |
| | sequence | | AGAATCTCCAGGGCAGAGTCATCATGACCACAGACACATCCACGAG |
| | | | CACAGCCTACATGGAGCTGAGGAGCCTGAGATCTGACGACACGGCC |
| | | | GTTTATTACTGTGCGAGGAGCAGTGGCCACTACTACTACTACGGTA |
| | | | TGGACGTCTGGGGCCAAGGGACCACGGTCACCGTCTCCTCAGCCAG |
| | | | CACCAAGGGCCCCTCTGTGTTCCCTCTGGCCCCTTCCAGCAAGTCC |
| | | | ACCTCTGGCGGAACAGCCGCTCTGGGCTGCCTCGTGAAGGACTACT |
| | | | TCCCCGAGCCTGTGACCGTGTCCTGGAACTCTGGCGCTCTGACCAG |
| | | | CGGAGTGCACACCTTCCCTGCTGTGCTGCAGTCCTCCGGCCTGTAC |
| | | | TCCCTGTCCTCCGTCGTGACCGTGCCTTCCAGCTCTCTGGGCACCC |
| | | | AGACCTACATCTGCAACGTGAACCACAAGCCCTCCAACACCAAGGT |
| | | | GGACAAGAAGGTGGAACCCAAGTCCTGCGACAAGACCCACACCTGT |
| | | | CCCCCTTGTCCTGCCCCTGAACTGCTGGGCGGACCTTCCGTGTTCC |
| | | | TGTTCCCCCCAAAGCCCAAGGACACCCTGATGATCTCCCGGACCCC |
| | | | CGAAGTGACCTGCGTGGTGGTGGATGTGTCCCACGAGGACCCTGAA |
| | | | GTGAAGTTCAATTGGTACGTGGACGGCGTGGAAGTGCACAACGCCA |
| | | | AGACCAAGCCTAGAGAGGAACAGTACAACTCCACCTACCGGGTGGT |
| | | | GTCCGTGCTGACCGTGCTGCACCAGGATTGGCTGAACGGCAAAGAG |
| | | | TACAAGTGCAAGGTGTCCAACAAGGCCCTGCCTGCCCCCATCGAAA |
| | | | AGACCATCTCCAAGGCCAAGGGCCAGCCCCGGGAACCCCAGGTGTA |
| | | | CACACTGCCCCCTAGCAGGGACGAGCTGACCAAGAACCAGGTGTCC |
| | | | CTGACCTGTCTCGTGAAAGGCTTCTACCCCTCCGATATCGCCGTGG |
| | | | AATGGGAGTCCAACGGCCAGCCTGAGAACAACTACAAGACCACCCC |
| | | | CCCTGTGCTGGACTCCGACGGCTCATTCTTCCTGTACAGCAAGCTG |
| | | | ACAGTGGACAAGTCCCGGTGGCAGCAGGGCAACGTGTTCTCCTGCT |
| | | | CCGTGATGCACGAGGCCCTGCACAACCACTACACCCAGAAGTCCCT |
| | | | GTCCCTGAGCCCCGGCAAGTGATGA |
| 370 | STIM001 | Amino acid sequence | QSLLHSNEYNY |
| | -CDRL1 | of CDRL1 of STIM001 | |
| | | using IMGT | |
| 371 | STIM001 | Amino acid sequence | LGS |
| | -CDRL2 | of CDRL2 of STIM001 | |
| | | using IMGT | |
| 372 | STIM001 | Amino acid sequence | MQSLQTPLT |
| | -CDRL3 | of CDRL3 of STIM001 | |
| | | using IMGT | |
| 373 | STIM001 | Amino acid sequence | DIVMTQSPLSLPVTPGEPASISCRSSQSLLHSNEYNYLDWYLQKPG |
| | -Light | of V_{L} of STIM001 | QSPQLLIFLGSNRASGVPDRFSGSGSGTDFTLKITRVEAEDVGIYY |
| | chain | | CMQSLQTPLTFGGGTKVEIK |
| | variable | | |
| | region | | |
| 374 | STIM001 | Nucleic acid | GATATTGTGATGACTCAGTCTCCACTCTCCCTGCCCGTCACCCCTG |
| | -Light | sequence of V_{L} of | GAGAGCCGGCCTCCATCTCCTGCAGGTCTAGTCAGAGCCTCCTGCA |
| | chain | STIM001 | TAGTAATGAATACAACTATTTGGATTGGTACCTGCAGAAGCCAGGG |
| | variable | | CAGTCTCCACAGCTCCTGATCTTTTTGGGTTCTAATCGGGCCTCCG |
| | region | | GGGTCCCTGACAGGTTCAGTGGCAGTGGATCAGGCACAGATTTTAC |
| | | | ACTGAAAATCACCAGAGTGGAGGCTGAGGATGTTGGAATTTATTAC |
| | | | TGCATGCAATCTCTACAAACTCCGCTCACTTTCGGCGGAGGGACCA |
| | | | AGGTGGAGATCAAA |
| 375 | STIM001 | Amino acid sequence | DIVMTQSPLSLPVTPGEPASISCRSSQSLLHSNEYNYLDWYLQKPG |
| | -full | of STIM001 light | QSPQLLIFLGSNRASGVPDRFSGSGSGTDFTLKITRVEAEDVGIYY |
| | light | chain | CMQSLQTPLTFGGGTKVEIK |
| | chain | | RTVAAPSVFIFPPSDEQLKSGTASVVCLLNNFYPREAKVQWKVDNA |
| | sequence | | LQSGNSQESVTEQDSKDSTYSLSSTLTLSKADYEKHKVYACEVTHQ |
| | | | GLSSPVTKSENRGEC |
| 376 | STIM001 | Nucleic acid | GATATTGTGATGACTCAGTCTCCACTCTCCCTGCCCGTCACCCCTG |
| | -full | sequence of STIM001 | GAGAGCCGGCCTCCATCTCCTGCAGGTCTAGTCAGAGCCTCCTGCA |
| | light | light chain | TAGTAATGAATACAACTATTTGGATTGGTACCTGCAGAAGCCAGGG |
| | chain | | CAGTCTCCACAGCTCCTGATCTTTTTGGGTTCTAATCGGGCCTCCG |
| | sequence | | GGGTCCCTGACAGGTTCAGTGGCAGTGGATCAGGCACAGATTTTAC |
| | | | ACTGAAAATCACCAGAGTGGAGGCTGAGGATGTTGGAATTTATTAC |
| | | | TGCATGCAATCTCTACAAACTCCGCTCACTTTCGGCGGAGGGACCA |
| | | | AGGTGGAGATCAAAcgtacggtggccgctccctccgtgttcatctt |
| | | | cccaccttccgacgagcagctgaagtccggcaccgcttctgtcgtg |
| | | | tgcctgctgaacaacttctacccccgcgaggccaaggtgcagtgga |
| | | | aggtggacaacgccctgcagtccggcaactcccaggaatccgtgac |
| | | | cgagcaggactccaaggacagcacctactccctgtcctccaccctg |
| | | | accctgtccaaggccgactacgagaagcacaaggtgtacgcctgcg |
| | | | aagtgacccaccagggcctgtctagccccgtgaccaagtctttcaa |
| | | | ccggggcgagtgt |
| 377 | STIM002 | Amino acid sequence | GYTFTSYG |
| | -CDRH1 | of CDRH1 of STIM002 | |
| | | using IMGT | |
| 378 | STIM002 | Amino acid sequence | ISAYNGNT |
| | -CDRH2 | of CDRH2 of STIM002 | |
| | | using IMGT | |
| 379 | STIM002 | Amino acid sequence | ARSTYFYGSGTLYGMDV |
| | -CDRH3 | of CDRH3 of STIM002 | |
| | | using IMGT | |
| 380 | STIM002 | Amino acid sequence | QVQLVQSGGEVKKPGASVKVSCKASGYTFTSYGFSWVRQAPGQGLE |
| | -Heavy | of V_{H} of STIM002 | WMGWISAYNGNTNYAQKLQGRVTMTTDTSTSTAYMELRSLRSDDTA |
| | chain | | VYYCARSTYFYGSGTLYGMDVWGQGTTVTVSS |
| | variable | | |
| | region | | |
| 381 | STIM002 | Nucleic acid | CAGGTTCAACTGGTGCAGTCTGGAGGTGAGGTGAAGAAGCCTGGGG |
| | -Heavy | sequence of V_{H} of | CCTCAGTGAAGGTCTCCTGCAAGGCTTCTGGTTACACCTTTACCAG |
| | chain | STIM002 | CTATGGTTTCAGCTGGGTGCGACAGGCCCCTGGACAAGGACTAGAG |
| | variable | | TGGATGGGATGGATCAGCGCTTACAATGGTAACACAAACTATGCAC |
| | region | | AGAAGCTCCAGGGCAGAGTCACCATGACCACAGACACATCCACGAG |
| | | | CACAGCCTACATGGAGCTGAGGAGCTTGAGATCTGACGACACGGCC |
| | | | GTGTATTACTGTGCGAGATCTACGTATTTCTATGGTTCGGGGACCC |
| | | | TCTACGGTATGGACGTCTGGGGCCAAGGGACCACGGTCACCGTCTC |
| | | | CTCA |
| 382 | STIM002 | Amino acid sequence | QVQLVQSGGEVKKPGASVKVSCKASGYTFTSYGFSWVRQAPGQGLE |
| | -full | of STIM002 heavy | WMGWISAYNGNTNYAQKLQGRVTMTTDTSTSTAYMELRSLRSDDTA |
| | heavy | chain | VYYCARSTYFYGSGTLYGMDVWGQGTTVTVSSASTKGPSVFPLAPS |
| | chain | | SKSTSGGTAALGCLVKDYFPEPVTVSWNSGALTSGVHTFPAVLQSS |
| | sequence | | GLYSLSSVVTVPSSSLGTQTYICNVNHKPSNTKVDKKVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYNSTYRVVSVLTVLHQDWLN |
| | | | GKEYKCKVSNKALPAPIEKTISKAKGQPREPQVYTLPPSRDELTKN |
| | | | QVSLTCLVKGFYPSDIAVEWESNGQPENNYKTTPPVLDSDGSFFLY |
| | | | SKLTVDKSRWQQGNVFSCSVMHEALHNHYTQKSLSLSPGK |
| | STIM002 | | CAGGTTCAACTGGTGCAGTCTGGAGGTGAGGTGAAGAAGCCTGGGG |
| 383 | -full | Nucleic acid | CCTCAGTGAAGGTCTCCTGCAAGGCTTCTGGTTACACCTTTACCAG |
| | heavy | sequence of STIM002 | CTATGGTTTCAGCTGGGTGCGACAGGCCCCTGGACAAGGACTAGAG |
| | chain | heavy chain | TGGATGGGATGGATCAGCGCTTACAATGGTAACACAAACTATGCAC |
| | sequence | | AGAAGCTCCAGGGCAGAGTCACCATGACCACAGACACATCCACGAG |
| | | | CACAGCCTACATGGAGCTGAGGAGCTTGAGATCTGACGACACGGCC |
| | | | GTGTATTACTGTGCGAGATCTACGTATTTCTATGGTTCGGGGACCC |
| | | | TCTACGGTATGGACGTCTGGGGCCAAGGGACCACGGTCACCGTCTC |
| | | | CTCA |
| | | | GCCAGCACCAAGGGCCCCTCTGTGTTCCCTCTGGCCCCTTCCAGCA |
| | | | AGTCCACCTCTGGCGGAACAGCCGCTCTGGGCTGCCTCGTGAAGGA |
| | | | CTACTTCCCCGAGCCTGTGACCGTGTCCTGGAACTCTGGCGCTCTG |
| | | | ACCAGCGGAGTGCACACCTTCCCTGCTGTGCTGCAGTCCTCCGGCC |
| | | | TGTACTCCCTGTCCTCCGTCGTGACCGTGCCTTCCAGCTCTCTGGG |
| | | | CACCCAGACCTACATCTGCAACGTGAACCACAAGCCCTCCAACACC |
| | | | AAGGTGGACAAGAAGGTGGAACCCAAGTCCTGCGACAAGACCCACA |
| | | | CCTGTCCCCCTTGTCCTGCCCCTGAACTGCTGGGCGGACCTTCCGT |
| | | | GTTCCTGTTCCCCCCAAAGCCCAAGGACACCCTGATGATCTCCCGG |
| | | | ACCCCCGAAGTGACCTGCGTGGTGGTGGATGTGTCCCACGAGGACC |
| | | | CTGAAGTGAAGTTCAATTGGTACGTGGACGGCGTGGAAGTGCACAA |
| | | | CGCCAAGACCAAGCCTAGAGAGGAACAGTACAACTCCACCTACCGG |
| | | | GTGGTGTCCGTGCTGACCGTGCTGCACCAGGATTGGCTGAACGGCA |
| | | | AAGAGTACAAGTGCAAGGTGTCCAACAAGGCCCTGCCTGCCCCCAT |
| | | | CGAAAAGACCATCTCCAAGGCCAAGGGCCAGCCCCGGGAACCCCAG |
| | | | GTGTACACACTGCCCCCTAGCAGGGACGAGCTGACCAAGAACCAGG |
| | | | TGTCCCTGACCTGTCTCGTGAAAGGCTTCTACCCCTCCGATATCGC |
| | | | CGTGGAATGGGAGTCCAACGGCCAGCCTGAGAACAACTACAAGACC |
| | | | ACCCCCCCTGTGCTGGACTCCGACGGCTCATTCTTCCTGTACAGCA |
| | | | AGCTGACAGTGGACAAGTCCCGGTGGCAGCAGGGCAACGTGTTCTC |
| | | | CTGCTCCGTGATGCACGAGGCCCTGCACAACCACTACACCCAGAAG |
| | | | TCCCTGTCCCTGAGCCCCGGCAAGTGATGA |
| 384 | STIM002 | Amino acid sequence | QSLLHSDGYNY |
| | -CDRL1 | of CDRL1 of STIM002 | |
| | | using IMGT | |
| 385 | STIM002 | Amino acid sequence | LGS |
| | -CDRL2 | of CDRL2 of STIM002 | |
| | | using IMGT | |
| 386 | STIM002 | Amino acid sequence | MQALQTPLS |
| | -CDRL3 | of CDRL3 of STIM002 | |
| | | using IMGT | |
| 387 | STIM002 | Amino acid sequence | DIVMTQSPLSLPVTPGEPASISCRSSQSLLHSDGYNYLDWYLQKPG |
| | -Light | of V_{L} of STIM002 | QSPQLLIYLGSTRASGFPDRESGSGSGTDFTLKISRVEAEDVGVYY |
| | chain | | CMQALQTPLSFGQGTKLEIK |
| | variable | | |
| | region | | |
| 388 | STIM002 | Nucleic acid | GATATTGTGATGACTCAGTCTCCACTCTCCCTGCCCGTCACCCCTG |
| | Light | sequence of V_{L} of | GAGAGCCGGCCTCCATCTCCTGCAGGTCTAGTCAGAGCCTCCTGCA |
| | chain | STIM002 | TAGTGATGGATACAACTGTTTGGATTGGTACCTGCAGAAGCCAGGG |
| | variable | | CAGTCTCCACAGCTCCTGATCTATTTGGGTTCTACTCGGGCCTCCG |
| | region | | GGTTCCCTGACAGGTTCAGTGGCAGTGGATCAGGCACAGATTTTAC |
| | | | ACTGAAAATCAGCAGAGTGGAGGCTGAGGATGTTGGGGTTTATTAC |
| | | | TGCATGCAAGCTCTACAAACTCCGTGCAGTTTTGGCCAGGGGACCA |
| | | | AGCTGGAGATCAAA |
| 389 | STIM002 | Amino acid sequence | DIVMTQSPLSLPVTPGEPASISCRSSQSLLHSDGYNYLDWYLQKPG |
| | -full | of STIM002 light | QSPQLLIYLGSTRASGFPDRFSGSGSGTDFTLKISRVEAEDVGVYY |
| | light | chain | CMQALQTPLSFGQGTKLEIK |
| | chain | | RTVAAPSVFIFPPSDEQLKSGTASVVCLLNNFYPREAKVQWKVDNA |
| | sequence | | LQSGNSQESVTEQDSKDSTYSLSSTLTLSKADYEKHKVYACEVTHQ |
| | | | GLSSPVTKSENRGEC |
| 390 | STIM002 | Nucleic acid | GATATTGTGATGACTCAGTCTCCACTCTCCCTGCCCGTCACCCCTG |
| | -full | sequence of STIM002 | GAGAGCCGGCCTCCATCTCCTGCAGGTCTAGTCAGAGCCTCCTGCA |
| | light | light chain | TAGTGATGGATACAACTGTTTGGATTGGTACCTGCAGAAGCCAGGG |
| | chain | | CAGTCTCCACAGCTCCTGATCTATTTGGGTTCTACTCGGGCCTCCG |
| | sequence | | GGTTCCCTGACAGGTTCAGTGGCAGTGGATCAGGCACAGATTTTAC |
| | | | ACTGAAAATCAGCAGAGTGGAGGCTGAGGATGTTGGGGTTTATTAC |
| | | | TGCATGCAAGCTCTACAAACTCCGTGCAGTTTTGGCCAGGGGACCA |
| | | | AGCTGGAGATCAAAcgtacggtggccgctccctccgtgttcatctt |
| | | | cccaccttccgacgagcagctgaagtccggcaccgcttctgtcgtg |
| | | | tgcctgctgaacaacttctacccccgcgaggccaaggtgcagtgga |
| | | | aggtggacaacgccctgcagtccggcaactcccaggaatccgtgac |
| | | | cgagcaggactccaaggacagcacctactccctgtcctccaccctg |
| | | | accctgtccaaggccgactacgagaagcacaaggtgtacgcctgcg |
| | | | aagtgacccaccagggcctgtctagccccgtgaccaagtctttcaa |
| | | | ccggggcgagtgt |
| 391 | STIM002- | Amino acid sequence | GYTFTSYG |
| | B- | of CDRH1 of | |
| | CDRH1 | STIM002-B using | |
| | | IMGT | |
| 392 | STIM002- | Amino acid sequence | ISAYNGNT |
| | B- | of CDRH2 of | |
| | CDRH2 | STIM002-B using | |
| | | IMGT | |
| 393 | STIM002- | Amino acid sequence | ARSTYFYGSGTLYGMDV |
| | B- | of CDRH3 of | |
| | CDRH3 | STIM002-B using | |
| | | IMGT | |
| 394 | STIM002- | Amino acid sequence | QVQLVQSGGEVKKPGASVKVSCKASGYTFTSYGFSWVRQAPGQGLE |
| | B- | of V_{H} of STIM002-B | WMGWISAYNGNTNYAQKLQGRVTMTTDTSTSTAYMELRSLRSDDTA |
| | Heavy | | VYYCARSTYFYGSGTLYGMDVWGQGTTVTVSS |
| | chain | | |
| | variable | | |
| | region | | |
| 395 | STIM002- | Nucleic acid | CAGGTTCAACTGGTGCAGTCTGGAGGTGAGGTGAAGAAGCCTGGGG |
| | B- | sequence of V_{H} of | CCTCAGTGAAGGTCTCCTGCAAGGCTTCTGGTTACACCTTTACCAG |
| | Heavy | STIM002-B | CTATGGTTTCAGCTGGGTGCGACAGGCCCCTGGACAAGGACTAGAG |
| | chain | | TGGATGGGATGGATCAGCGCTTACAATGGTAACACAAACTATGCAC |
| | variable | | AGAAGCTCCAGGGCAGAGTCACCATGACCACAGACACATCCACGAG |
| | region | | CACAGCCTACATGGAGCTGAGGAGCTTGAGATCTGACGACACGGCC |
| | | | GTGTATTACTGTGCGAGATCTACGTATTTCTATGGTTCGGGGACCC |
| | | | TCTACGGTATGGACGTCTGGGGCCAAGGGACCACGGTCACCGTCTC |
| | | | CTCA |
| 396 | STIM002- | Amino acid sequence | QVQLVQSGGEVKKPGASVKVSCKASGYTFTSYGFSWVRQAPGQGLE |
| | B-full | of STIM002-B heavy | WMGWISAYNGNTNYAQKLQGRVTMTTDTSTSTAYMELRSLRSDDTA |
| | heavy | chain | VYYCARSTYFYGSGTLYGMDVWGQGTTVTVSSASTKGPSVFPLAPS |
| | chain | | SKSTSGGTAALGCLVKDYFPEPVTVSWNSGALTSGVHTFPAVLQSS |
| | sequence | | GLYSLSSVVTVPSSSLGTQTYICNVNHKPSNTKVDKKVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYNSTYRVVSVLTVLHQDWLN |
| | | | GKEYKCKVSNKALPAPIEKTISKAKGQPREPQVYTLPPSRDELTKN |
| | | | QVSLTCLVKGFYPSDIAVEWESNGQPENNYKTTPPVLDSDGSFFLY |
| | | | SKLTVDKSRWQQGNVFSCSVMHEALHNHYTQKSLSLSPGK |
| 397 | STIM002- | Nucleic acid | CAGGTTCAACTGGTGCAGTCTGGAGGTGAGGTGAAGAAGCCTGGGG |
| | B-full | sequence of | CCTCAGTGAAGGTCTCCTGCAAGGCTTCTGGTTACACCTTTACCAG |
| | heavy | STIM002-B heavy | CTATGGTTTCAGCTGGGTGCGACAGGCCCCTGGACAAGGACTAGAG |
| | chain | chain | TGGATGGGATGGATCAGCGCTTACAATGGTAACACAAACTATGCAC |
| | sequence | | AGAAGCTCCAGGGCAGAGTCACCATGACCACAGACACATCCACGAG |
| | | | CACAGCCTACATGGAGCTGAGGAGCTTGAGATCTGACGACACGGCC |
| | | | GTGTATTACTGTGCGAGATCTACGTATTTCTATGGTTCGGGGACCC |
| | | | TCTACGGTATGGACGTCTGGGGCCAAGGGACCACGGTCACCGTCTC |
| | | | CTCAGCCAGCACCAAGGGCCCCTCTGTGTTCCCTCTGGCCCCTTCC |
| | | | AGCAAGTCCACCTCTGGCGGAACAGCCGCTCTGGGCTGCCTCGTGA |
| | | | AGGACTACTTCCCCGAGCCTGTGACCGTGTCCTGGAACTCTGGCGC |
| | | | TCTGACCAGCGGAGTGCACACCTTCCCTGCTGTGCTGCAGTCCTCC |
| | | | GGCCTGTACTCCCTGTCCTCCGTCGTGACCGTGCCTTCCAGCTCTC |
| | | | TGGGCACCCAGACCTACATCTGCAACGTGAACCACAAGCCCTCCAA |
| | | | CACCAAGGTGGACAAGAAGGTGGAACCCAAGTCCTGCGACAAGACC |
| | | | CACACCTGTCCCCCTTGTCCTGCCCCTGAACTGCTGGGCGGACCTT |
| | | | CCGTGTTCCTGTTCCCCCCAAAGCCCAAGGACACCCTGATGATCTC |
| | | | CCGGACCCCCGAAGTGACCTGCGTGGTGGTGGATGTGTCCCACGAG |
| | | | GACCCTGAAGTGAAGTTCAATTGGTACGTGGACGGCGTGGAAGTGC |
| | | | ACAACGCCAAGACCAAGCCTAGAGAGGAACAGTACAACTCCACCTA |
| | | | CCGGGTGGTGTCCGTGCTGACCGTGCTGCACCAGGATTGGCTGAAC |
| | | | GGCAAAGAGTACAAGTGCAAGGTGTCCAACAAGGCCCTGCCTGCCC |
| | | | CCATCGAAAAGACCATCTCCAAGGCCAAGGGCCAGCCCCGGGAACC |
| | | | CCAGGTGTACACACTGCCCCCTAGCAGGGACGAGCTGACCAAGAAC |
| | | | CAGGTGTCCCTGACCTGTCTCGTGAAAGGCTTCTACCCCTCCGATA |
| | | | TCGCCGTGGAATGGGAGTCCAACGGCCAGCCTGAGAACAACTACAA |
| | | | GACCACCCCCCCTGTGCTGGACTCCGACGGCTCATTCTTCCTGTAC |
| | | | AGCAAGCTGACAGTGGACAAGTCCCGGTGGCAGCAGGGCAACGTGT |
| | | | TCTCCTGCTCCGTGATGCACGAGGCCCTGCACAACCACTACACCCA |
| | | | GAAGTCCCTGTCCCTGAGCCCCGGCAAGTGATGA |
| 398 | STIM002- | Amino acid sequence | QSLLHSDGYNC |
| | B- | of CDRL1 of | |
| | CDRL1 | STIM002-B using | |
| | | IMGT | |
| 399 | STIM002- | Amino acid sequence | LGS |
| | B- | of CDRL2 of | |
| | CDRL2 | STIM002-B using | |
| | | IMGT | |
| 400 | STIM002- | Amino acid sequence | MQALQTPCS |
| | B- | of CDRL3 of | |
| | CDRL3 | STIM002-B using | |
| | | IMGT | |
| 401 | STIM002- | Amino acid sequence | DIVMTQSPLSLPVTPGEPASISCRSSQSLLHSDGYNCLDWYLQKPG |
| | B- | of V_{L} of STIM002-B | QSPQLLIYLGSTRASGFPDRESGSGSGTDFTLKISRVEAEDVGVYY |
| | Light | | CMQALQTPCSFGQGTKLEIK |
| | chain | | |
| | variable | | |
| | region | | |
| 402 | STIM002- | Nucleic acid | GATATTGTGATGACTCAGTCTCCACTCTCCCTGCCCGTCACCCCTG |
| | B- | sequence of V_{L} of | GAGAGCCGGCCTCCATCTCCTGCAGGTCTAGTCAGAGCCTCCTGCA |
| | Light | STIM002-B | TAGTGATGGATACAACTGTTTGGATTGGTACCTGCAGAAGCCAGGG |
| | chain | | CAGTCTCCACAGCTCCTGATCTATTTGGGTTCTACTCGGGCCTCCG |
| | variable | | GGTTCCCTGACAGGTTCAGTGGCAGTGGATCAGGCACAGATTTTAC |
| | region | | ACTGAAAATCAGCAGAGTGGAGGCTGAGGATGTTGGGGTTTATTAC |
| | | | TGCATGCAAGCTCTACAAACTCCGTGCAGTTTTGGCCAGGGGACCA |
| | | | AGCTGGAGATCAAA |
| 403 | STIM002- | Amino acid sequence | DIVMTQSPLSLPVTPGEPASISCRSSQSLLHSDGYNCLDWYLQKPG |
| | B-full | of STIM002-B light | QSPQLLIYLGSTRASGFPDRESGSGSGTDFTLKISRVEAEDVGVYY |
| | light | chain | CMQALQTPCSFGQGTKLEIK |
| | chain | | RTVAAPSVFIFPPSDEQLKSGTASVVCLLNNFYPREAKVQWKVDNA |
| | sequence | | LQSGNSQESVTEQDSKDSTYSLSSTLTLSKADYEKHKVYACEVTHQ |
| | | | GLSSPVTKSENRGEC |
| 404 | STIM002- | Nucleic acid | GATATTGTGATGACTCAGTCTCCACTCTCCCTGCCCGTCACCCCTG |
| | B-full | sequence of | GAGAGCCGGCCTCCATCTCCTGCAGGTCTAGTCAGAGCCTCCTGCA |
| | light | STIM002-B light | TAGTGATGGATACAACTGTTTGGATTGGTACCTGCAGAAGCCAGGG |
| | chain | chain | CAGTCTCCACAGCTCCTGATCTATTTGGGTTCTACTCGGGCCTCCG |
| | sequence | | GGTTCCCTGACAGGTTCAGTGGCAGTGGATCAGGCACAGATTTTAC |
| | | | ACTGAAAATCAGCAGAGTGGAGGCTGAGGATGTTGGGGTTTATTAC |
| | | | TGCATGCAAGCTCTACAAACTCCGTGCAGTTTTGGCCAGGGGACCA |
| | | | AGCTGGAGATCAAAcgtacggtggccgctccctccgtgttcatctt |
| | | | cccaccttccgacgagcagctgaagtccggcaccgcttctgtcgtg |
| | | | tgcctgctgaacaacttctacccccgcgaggccaaggtgcagtgga |
| | | | aggtggacaacgccctgcagtccggcaactcccaggaatccgtgac |
| | | | cgagcaggactccaaggacagcacctactccctgtcctccaccctg |
| | | | accctgtccaaggccgactacgagaagcacaaggtgtacgcctgcg |
| | | | aagtgacccaccagggcctgtctagccccgtgaccaagtotttcaa |
| | | | ccggggcgagtgt |
| 405 | STIM003 | Amino acid sequence | GVTEDDYG |
| | -CDRH1 | of CDRH1 of STIM003 | |
| | | using IMGT | |
| 406 | STIM003 | Amino acid sequence | INWNGGDT |
| | -CDRH2 | of CDRH2 of STIM003 | |
| | | using IMGT | |
| 407 | STIM003 | Amino acid sequence | ARDFYGSGSYYHVPFDY |
| | -CDRH3 | of CDRH3 of STIM003 | |
| | | using IMGT | |
| 408 | STIM003 | Amino acid sequence | EVQLVESGGGVVRPGGSLRLSCVASGVTFDDYGMSWVRQAPGKGLE |
| | -Heavy | of V_{H} of STIM003 | WVSGINWNGGDTDYSDSVKGRFTISRDNAKNSLYLQMNSLRAEDTA |
| | chain | | LYYCARDFYGSGSYYHVPFDYWGQGILVTVSS |
| | variable | | |
| | region | | |
| 409 | STIM003 | Nucleic acid | GAGGTGCAGCTGGTGGAGTCTGGGGGAGGTGTGGTACGGCCTGGGG |
| | -Heavy | sequence of V_{H} of | GGTCCCTGAGACTCTCCTGTGTAGCCTCTGGAGTCACCTTTGATGA |
| | chain | STIM003 | TTATGGCATGAGCTGGGTCCGCCAAGCTCCAGGGAAGGGGCTGGAR |
| | variable | | TGGGTCTCTGGTATTAATTGGAATGGTGGCGACACAGATTATTCAG |
| | region | | ACTCTGTGAAGGGCCGATTCACCATCTCCAGAGACAACGCCAAGAA |
| | | | CTCCCTGTATCTACAAATGAATAGTCTGAGAGCCGAGGACACGGCC |
| | | | TTGTATTACTGTGCGAGGGATTTCTATGGTTCGGGGAGTTATTATC |
| | | | ACGTTCCTTTTGACTACTGGGGCCAGGGAATCCTGGTCACCGTCTC |
| | | | CTCA |
| 410 | STIM003 | Amino acid sequence | EVQLVESGGGVVRPGGSLRLSCVASGVTEDDYGMSWVRQAPGKGLE |
| | -full | of STIM003 heavy | WVSGINWNGGDTDYSDSVKGRFTISRDNAKNSLYLQMNSLRAEDTA |
| | heavy | chain | LYYCARDFYGSGSYYHVPFDYWGQGILVTVSSASTKGPSVFPLAPS |
| | chain | | SKSTSGGTAALGCLVKDYFPEPVTVSWNSGALTSGVHTFPAVLQSS |
| | sequence | | GLYSLSSVVTVPSSSLGTQTYICNVNHKPSNTKVDKKVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYNSTYRVVSVLTVLHQDWLN |
| | | | GKEYKCKVSNKALPAPIEKTISKAKGQPREPQVYTLPPSRDELTKN |
| | | | QVSLTCLVKGFYPSDIAVEWESNGQPENNYKTTPPVLDSDGSFFLY |
| | | | SKLTVDKSRWQQGNVFSCSVMHEALHNHYTQKSLSLSPGK |
| 411 | STIM003 | Nucleic acid | GAGGTGCAGCTGGTGGAGTCTGGGGGAGGTGTGGTACGGCCTGGGG |
| | -full | sequence of STIM003 | GGTCCCTGAGACTCTCCTGTGTAGCCTCTGGAGTCACCTTTGATGA |
| | heavy | heavy chain | TTATGGCATGAGCTGGGTCCGCCAAGCTCCAGGGAAGGGGCTGGAR |
| | chain | | TGGGTCTCTGGTATTAATTGGAATGGTGGCGACACAGATTATTCAG |
| | sequence | | ACTCTGTGAAGGGCCGATTCACCATCTCCAGAGACAACGCCAAGAA |
| | | | CTCCCTGTATCTACAAATGAATAGTCTGAGAGCCGAGGACACGGCC |
| | | | TTGTATTACTGTGCGAGGGATTTCTATGGTTCGGGGAGTTATTATC |
| | | | ACGTTCCTTTTGACTACTGGGGCCAGGGAATCCTGGTCACCGTCTC |
| | | | CTCAGCCAGCACCAAGGGCCCCTCTGTGTTCCCTCTGGCCCCTTCC |
| | | | AGCAAGTCCACCTCTGGCGGAACAGCCGCTCTGGGCTGCCTCGTGA |
| | | | AGGACTACTTCCCCGAGCCTGTGACCGTGTCCTGGAACTCTGGCGC |
| | | | TCTGACCAGCGGAGTGCACACCTTCCCTGCTGTGCTGCAGTCCTCC |
| | | | GGCCTGTACTCCCTGTCCTCCGTCGTGACCGTGCCTTCCAGCTCTC |
| | | | TGGGCACCCAGACCTACATCTGCAACGTGAACCACAAGCCCTCCAA |
| | | | CACCAAGGTGGACAAGAAGGTGGAACCCAAGTCCTGCGACAAGACC |
| | | | CACACCTGTCCCCCTTGTCCTGCCCCTGAACTGCTGGGCGGACCTT |
| | | | CCGTGTTCCTGTTCCCCCCAAAGCCCAAGGACACCCTGATGATCTC |
| | | | CCGGACCCCCGAAGTGACCTGCGTGGTGGTGGATGTGTCCCACGAG |
| | | | GACCCTGAAGTGAAGTTCAATTGGTACGTGGACGGCGTGGAAGTGC |
| | | | ACAACGCCAAGACCAAGCCTAGAGAGGAACAGTACAACTCCACCTA |
| | | | CCGGGTGGTGTCCGTGCTGACCGTGCTGCACCAGGATTGGCTGAAC |
| | | | GGCAAAGAGTACAAGTGCAAGGTGTCCAACAAGGCCCTGCCTGCCC |
| | | | CCATCGAAAAGACCATCTCCAAGGCCAAGGGCCAGCCCCGGGAACC |
| | | | CCAGGTGTACACACTGCCCCCTAGCAGGGACGAGCTGACCAAGAAC |
| | | | CAGGTGTCCCTGACCTGTCTCGTGAAAGGCTTCTACCCCTCCGATA |
| | | | TCGCCGTGGAATGGGAGTCCAACGGCCAGCCTGAGAACAACTACAA |
| | | | GACCACCCCCCCTGTGCTGGACTCCGACGGCTCATTCTTCCTGTAC |
| | | | AGCAAGCTGACAGTGGACAAGTCCCGGTGGCAGCAGGGCAACGTGT |
| | | | TCTCCTGCTCCGTGATGCACGAGGCCCTGCACAACCACTACACCCA |
| | | | GAAGTCCCTGTCCCTGAGCCCCGGCAAGTGATGA |
| 412 | STIM003 | Amino acid sequence | QSVSRSY |
| | -CDRL1 | of CDRL1 of STIM003 | |
| | | using IMGT | |
| 413 | STIM003 | Amino acid sequence | GAS |
| | -CDRL2 | of CDRL2 of STIM003 | |
| | | using IMGT | |
| 414 | STIM003 | Amino acid sequence | HQYDMSPFT |
| | -CDRL3 | of CDRL3 of STIM003 | |
| | | using IMGT | |
| 415 | STIM003 | Amino acid sequence | EIVLTQSPGTLSLSPGERATLSCRASQSVSRSYLAWYQQKRGQAPR |
| | -Light | of V_{L} of STIM003 | LLIYGASSRATGIPDRFSGDGSGTDFTLSISRLEPEDFAVYYCHQY |
| | chain | | DMSPFTFGPGTKVDIK |
| | variable | | |
| | region | | |
| 416 | STIM003 | Nucleic acid | GAAATTGTGTTGACGCAGTCTCCAGGGACCCTGTCTTTGTCTCCAG |
| | -Light | sequence of V_{L} of | GGGAAAGAGCCACCCTCTCCTGCAGGGCCAGTCAGAGTGTTAGCAG |
| | chain | STIM003 | AAGCTACTTAGCCTGGTACCAGCAGAAACGTGGCCAGGCTCCCAGG |
| | variable | | CTCCTCATCTATGGTGCATCCAGCAGGGCCACTGGCATCCCAGACA |
| | region | | GGTTCAGTGGCGATGGGTCTGGGACAGACTTCACTCTCTCCATCAG |
| | | | CAGACTGGAGCCTGAAGATTTTGCAGTGTATTACTGTCACCAGTAT |
| | | | GATATGTCACCATTCACTTTCGGCCCTGGGACCAAAGTGGATATCA |
| | | | AA |
| 417 | STIM003 | Amino acid sequence | EIVLTQSPGTLSLSPGERATLSCRASQSVSRSYLAWYQQKRGQAPR |
| | -full | of STIM003 light | LLIYGASSRATGIPDRFSGDGSGTDFTLSISRLEPEDFAVYYCHQY |
| | light | chain | DMSPFTFGPGTKVDIKRTVAAPSVFIFPPSDEQLKSGTASVVCLLN |
| | chain | | NFYPREAKVQWKVDNALQSGNSQESVTEQDSKDSTYSLSSTLTLSK |
| | sequence | | ADYEKHKVYACEVTHQGLSSPVTKSFNRGEC |
| 418 | STIM003 | Nucleic acid | GAAATTGTGTTGACGCAGTCTCCAGGGACCCTGTCTTTGTCTCCAG |
| | -full | sequence of STIM003 | GGGAAAGAGCCACCCTCTCCTGCAGGGCCAGTCAGAGTGTTAGCAG |
| | light | light chain | AAGCTACTTAGCCTGGTACCAGCAGAAACGTGGCCAGGCTCCCAGG |
| | chain | | CTCCTCATCTATGGTGCATCCAGCAGGGCCACTGGCATCCCAGACA |
| | sequence | | GGTTCAGTGGCGATGGGTCTGGGACAGACTTCACTCTCTCCATCAG |
| | | | CAGACTGGAGCCTGAAGATTTTGCAGTGTATTACTGTCACCAGTAT |
| | | | GATATGTCACCATTCACTTTCGGCCCTGGGACCAAAGTGGATATCA |
| | | | AAcgtacggtggccgctccctccgtgttcatcttcccaccttccga |
| | | | cgagcagctgaagtccggcaccgcttctgtcgtgtgcctgctgaac |
| | | | aacttctacccccgcgaggccaaggtgcagtggaaggtggacaacg |
| | | | ccctgcagtccggcaactcccaggaatccgtgaccgagcaggactc |
| | | | caaggacagcacctactccctgtcctccaccctgaccctgtccaag |
| | | | gccgactacgagaagcacaaggtgtacgcctgcgaagtgacccacc |
| | | | agggcctgtctagccccgtgaccaagtctttcaaccggggcgagtg |
| | | | t |
| 419 | STIM004 | Amino acid sequence | GLTEDDYG |
| | -CDRH1 | of CDRH1 of STIM004 | |
| | | using IMGT | |
| 420 | STIM004 | Amino acid sequence | INWNGDNT |
| | -CDRH2 | of CDRH2 of STIM004 | |
| | | using IMGT | |
| 421 | STIM004 | Amino acid sequence | ARDYYGSGSYYNVPFDY |
| | -CDRH3 | of CDRH3 of STIM004 | |
| | | using IMGT | |
| 422 | STIM004 | Amino acid sequence | EVQLVESGGGVVRPGGSLRLSCAASGLTEDDYGMSWVRQVPGKGLE |
| | -Heavy | of V_{H} of STIM004 | WVSGINWNGDNTDYADSVKGRFTISRDNAKNSLYLQMNSLRAEDTA |
| | chain | | LYYCARDYYGSGSYYNVPFDYWGQGTLVTVSS |
| | variable | | |
| | region | | |
| 423 | STIM004 | Nucleic acid | GAGGTGCAGCTGGTGGAGTCTGGGGGAGGTGTGGTACGGCCTGGGG |
| | -Heavy | sequence of V_{H} of | GGTCCCTGAGACTCTCCTGTGCAGCCTCTGGACTCACCTTTGATGA |
| | chain | STIM004 | TTATGGCATGAGCTGGGTCCGCCAAGTTCCAGGGAAGGGGCTGGAG |
| | variable | | TGGGTCTCTGGTATTAATTGGAATGGTGATAACACAGATTATGCAG |
| | region | | ACTCTGTGAAGGGCCGATTCACCATCTCCAGAGACAACGCCAAGAA |
| | | | CTCCCTGTATCTGCAAATGAACAGTCTGAGAGCCGAGGACACGGCC |
| | | | TTGTATTACTGTGCGAGGGATTACTATGGTTCGGGGAGTTATTATA |
| | | | ACGTTCCTTTTGACTACTGGGGCCAGGGAACCCTGGTCACCGTCTC |
| | | | CTCA |
| 424 | STIM004 | Amino acid sequence | EVQLVESGGGVVRPGGSLRLSCAASGLTEDDYGMSWVRQVPGKGLE |
| | -full | of STIM004 heavy | WVSGINWNGDNTDYADSVKGRFTISRDNAKNSLYLQMNSLRAEDTA |
| | heavy | chain | LYYCARDYYGSGSYYNVPFDYWGQGTLVTVSSASTKGPSVFPLAPS |
| | chain | | SKSTSGGTAALGCLVKDYFPEPVTVSWNSGALTSGVHTFPAVLQSS |
| | sequence | | GLYSLSSVVTVPSSSLGTQTYICNVNHKPSNTKVDKKVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYNSTYRVVSVLTVLHQDWLN |
| | | | GKEYKCKVSNKALPAPIEKTISKAKGQPREPQVYTLPPSRDELTKN |
| | | | QVSLTCLVKGFYPSDIAVEWESNGQPENNYKTTPPVLDSDGSFFLY |
| | | | SKLTVDKSRWQQGNVFSCSVMHEALHNHYTQKSLSLSPGK |
| 425 | STIM004 | Nucleic acid | GAGGTGCAGCTGGTGGAGTCTGGGGGAGGTGTGGTACGGCCTGGGG |
| | -full | sequence of STIM004 | GGTCCCTGAGACTCTCCTGTGCAGCCTCTGGACTCACCTTTGATGA |
| | heavy | heavy chain | TTATGGCATGAGCTGGGTCCGCCAAGTTCCAGGGAAGGGGCTGGAG |
| | chain | | TGGGTCTCTGGTATTAATTGGAATGGTGATAACACAGATTATGCAG |
| | sequence | | ACTCTGTGAAGGGCCGATTCACCATCTCCAGAGACAACGCCAAGAA |
| | | | CTCCCTGTATCTGCAAATGAACAGTCTGAGAGCCGAGGACACGGCC |
| | | | TTGTATTACTGTGCGAGGGATTACTATGGTTCGGGGAGTTATTATA |
| | | | ACGTTCCTTTTGACTACTGGGGCCAGGGAACCCTGGTCACCGTCTC |
| | | | CTCAGCCAGCACCAAGGGCCCCTCTGTGTTCCCTCTGGCCCCTTCC |
| | | | AGCAAGTCCACCTCTGGCGGAACAGCCGCTCTGGGCTGCCTCGTGA |
| | | | AGGACTACTTCCCCGAGCCTGTGACCGTGTCCTGGAACTCTGGCGC |
| | | | TCTGACCAGCGGAGTGCACACCTTCCCTGCTGTGCTGCAGTCCTCC |
| | | | GGCCTGTACTCCCTGTCCTCCGTCGTGACCGTGCCTTCCAGCTCTC |
| | | | TGGGCACCCAGACCTACATCTGCAACGTGAACCACAAGCCCTCCAA |
| | | | CACCAAGGTGGACAAGAAGGTGGAACCCAAGTCCTGCGACAAGACC |
| | | | CACACCTGTCCCCCTTGTCCTGCCCCTGAACTGCTGGGCGGACCTT |
| | | | CCGTGTTCCTGTTCCCCCCAAAGCCCAAGGACACCCTGATGATCTC |
| | | | CCGGACCCCCGAAGTGACCTGCGTGGTGGTGGATGTGTCCCACGAG |
| | | | GACCCTGAAGTGAAGTTCAATTGGTACGTGGACGGCGTGGAAGTGC |
| | | | ACAACGCCAAGACCAAGCCTAGAGAGGAACAGTACAACTCCACCTA |
| | | | CCGGGTGGTGTCCGTGCTGACCGTGCTGCACCAGGATTGGCTGAAC |
| | | | GGCAAAGAGTACAAGTGCAAGGTGTCCAACAAGGCCCTGCCTGCCC |
| | | | CCATCGAAAAGACCATCTCCAAGGCCAAGGGCCAGCCCCGGGAACC |
| | | | CCAGGTGTACACACTGCCCCCTAGCAGGGACGAGCTGACCAAGAAC |
| | | | CAGGTGTCCCTGACCTGTCTCGTGAAAGGCTTCTACCCCTCCGATA |
| | | | TCGCCGTGGAATGGGAGTCCAACGGCCAGCCTGAGAACAACTACAA |
| | | | GACCACCCCCCCTGTGCTGGACTCCGACGGCTCATTCTTCCTGTAC |
| | | | AGCAAGCTGACAGTGGACAAGTCCCGGTGGCAGCAGGGCAACGTGT |
| | | | TCTCCTGCTCCGTGATGCACGAGGCCCTGCACAACCACTACACCCA |
| | | | GAAGTCCCTGTCCCTGAGCCCCGGCAAGTGATGA |
| 426 | STIM004 | Amino acid sequence | QSVSSSY |
| | -CDRL1 | of CDRL1 of STIM004 | |
| | | using IMGT | |
| 427 | STIM004 | Amino acid sequence | GAS |
| | -CDRL2 | of CDRL2 of STIM004 | |
| | | using IMGT | |
| 428 | STIM004 | Amino acid sequence | QQYGSSPF |
| | -CDRL3 | of CDRL3 of STIM004 | |
| | | using IMGT | |
| 429 | STIM004- | Amino acid sequence | EIVLTQSPGTLSLSPGERATLSCRASQSVSSSYLAWYQQKPGQAPR |
| | Corrected | of corrected V_{L} of | LLIYGASSRATGIPDRFSGSGSGTDFTLTIRRLEPEDFAVYYCQQY |
| | light | STIM004 | GSSPFFGPGTKVDIK |
| | chain | | |
| | variable | | |
| | region | | |
| 430 | STIM004- | Nucleic acid | GAAATTGTGTTGACGCAGTCTCCAGGCACCCTGTCTTTGTCTCCAG |
| | Corrected | sequence of | GGGAAAGAGCCACCCTCTCCTGCAGGGCCAGTCAGAGTGTTAGCAG |
| | light | corrected V_{L} of | CAGCTACTTAGCCTGGTACCAGCAGAAACCTGGCCAGGCTCCCAGG |
| | chain | STIM004 | CTCCTCATATATGGTGCATCCAGCAGGGCCACTGGCATCCCAGACA |
| | variable | | GGTTCAGTGGCAGTGGGTCTGGGACAGACTTCACTCTCACCATCAG |
| | region | | AAGACTGGAGCCTGAAGATTTTGCAGTGTATTACTGTCAGCAGTAT |
| | | | GGTAGTTCACCATTCTTCGGCCCTGGGACCAAAGTGGATATCAAA |
| 431 | STIM004 | Nucleic acid | GAAATTGTGTTGACGCAGTCTCCAGGCACCCTGTCTTTGTCTCCAG |
| | -Light | sequence of V_{L} of | GGGAAAGAGCCACCCTCTCCTGCAGGGCCAGTCAGAGTGTTAGCAG |
| | chain | STIM004 | CAGCTACTTAGCCTGGTACCAGCAGAAACCTGGCCAGGCTCCCAGG |
| | variable | | CTCCTCATATATGGTGCATCCAGCAGGGCCACTGGCATCCCAGACA |
| | region | | GGTTCAGTGGCAGTGGGTCTGGGACAGACTTCACTCTCACCATCAG |
| | | | AAGACTGGAGCCTGAAGATTTTGCAGTGTATTACTGTCAGCAGTAT |
| | | | GGTAGTTCACCATTCACTTCGGCCCTGGGACCAAAGTGGATATCAA |
| | | | A |
| 432 | STIM004 | Amino acid sequence | EIVLTQSPGTLSLSPGERATLSCRASQSVSSSYLAWYQQKPGQAPR |
| | -full | of STIM004 light | LLIYGASSRATGIPDRFSGSGSGTDFTLTIRRLEPEDFAVYYCQQY |
| | corrected | chain | GSSPFFGPGTKVDIKRTVAAPSVFIFPPSDEQLKSGTASVVCLLNN |
| | light | | FYPREAKVQWKVDNALQSGNSQESVTEQDSKDSTYSLSSTLTLSKA |
| | chain | | DYEKHKVYACEVTHQGLSSPVTKSENRGEC |
| | sequence | | |
| 433 | STIM004 | Nucleic acid | GAAATTGTGTTGACGCAGTCTCCAGGCACCCTGTCTTTGTCTCCAG |
| | -full | sequence of | GGGAAAGAGCCACCCTCTCCTGCAGGGCCAGTCAGAGTGTTAGCAG |
| | corrected | corrected STIM004 | CAGCTACTTAGCCTGGTACCAGCAGAAACCTGGCCAGGCTCCCAGG |
| | light | light chain | CTCCTCATATATGGTGCATCCAGCAGGGCCACTGGCATCCCAGACA |
| | chain | | GGTTCAGTGGCAGTGGGTCTGGGACAGACTTCACTCTCACCATCAG |
| | sequence | | AAGACTGGAGCCTGAAGATTTTGCAGTGTATTACTGTCAGCAGTAT |
| | | | GGTAGTTCACCATTCTTCGGCCCTGGGACCAAAGTGGATATCAAAc |
| | | | gtacggtggccgctccctccgtgttcatcttcccaccttccgacga |
| | | | gcagctgaagtccggcaccgcttctgtcgtgtgcctgctgaacaac |
| | | | ttctacccccgcgaggccaaggtgcagtggaaggtggacaacgccc |
| | | | tgcagtccggcaactcccaggaatccgtgaccgagcaggactccaa |
| | | | ggacagcacctactccctgtcctccaccctgaccctgtccaaggcc |
| | | | gactacgagaagcacaaggtgtacgcctgcgaagtgacccaccagg |
| | | | gcctgtctagccccgtgaccaagtctttcaaccggggcgagtgt |
| 434 | STIM004 | Nucleic acid | GAAATTGTGTTGACGCAGTCTCCAGGCACCCTGTCTTTGTCTCCAG |
| | -full | sequence of STIM004 | GGGAAAGAGCCACCCTCTCCTGCAGGGCCAGTCAGAGTGTTAGCAG |
| | light | light chain | CAGCTACTTAGCCTGGTACCAGCAGAAACCTGGCCAGGCTCCCAGG |
| | chain | | CTCCTCATATATGGTGCATCCAGCAGGGCCACTGGCATCCCAGACA |
| | sequence | | GGTTCAGTGGCAGTGGGTCTGGGACAGACTTCACTCTCACCATCAG |
| | | | AAGACTGGAGCCTGAAGATTTTGCAGTGTATTACTGTCAGCAGTAT |
| | | | GGTAGTTCACCATTCACTTCGGCCCTGGGACCAAAGTGGATATCAA |
| | | | Acgtacggtggccgctccctccgtgttcatcttcccaccttccgac |
| | | | gagcagctgaagtccggcaccgcttctgtcgtgtgcctgctgaaca |
| | | | acttctacccccgcgaggccaaggtgcagtggaaggtggacaacgc |
| | | | cctgcagtccggcaactcccaggaatccgtgaccgagcaggactcc |
| | | | aaggacagcacctactccctgtcctccaccctgaccctgtccaagg |
| | | | ccgactacgagaagcacaaggtgtacgcctgcgaagtgacccacca |
| | | | gggcctgtctagccccgtgaccaagtctttcaaccggggcgagtgt |
| 435 | STIM005 | Amino acid sequence | GYTENSYG |
| | -CDRH1 | of CDRH1 of STIM005 | |
| | | using IMGT | |
| 436 | STIM005 | Amino acid sequence | ISVHNGNT |
| | -CDRH2 | of CDRH2 of STIM005 | |
| | | using IMGT | |
| 437 | STIM005 | Amino acid sequence | ARAGYDILTDESDAFDI |
| | -CDRH3 | of CDRH3 of STIM005 | |
| | | using IMGT | |
| 438 | STIM005 | Amino acid sequence | QVQLVQSGAEVKKPGASVKVSCKASGYTFNSYGIIWVRQAPGQGLE |
| | -Heavy | of V_{H} of STIM005 | WMGWISVHNGNTNCAQKLQGRVTMTTDTSTSTAYMELRSLRTDDTA |
| | chain | | VYYCARAGYDILTDFSDAFDIWGHGTMVTVSS |
| | variable | | |
| | region | | |
| 439 | STIM005 | Nucleic acid | CAGGTTCAGTTGGTGCAGTCTGGAGCTGAGGTGAAGAAGCCTGGGG |
| | -Heavy | sequence of V_{H} of | CCTCAGTGAAGGTCTCCTGCAAGGCTTCTGGTTACACCTTTAATAG |
| | chain | STIM005 | TTATGGTATCATCTGGGTGCGACAGGCCCCTGGACAAGGGCTTGAG |
| | variable | | TGGATGGGATGGATCAGCGTTCACAATGGTAACACAAACTGTGCAC |
| | region | | AGAAGCTCCAGGGTAGAGTCACCATGACCACAGACACATCCACGAG |
| | | | CACAGCCTACATGGAGCTGAGGAGCCTGAGAACTGACGACACGGCC |
| | | | GTGTATTACTGTGCGAGAGCGGGTTACGATATTTTGACTGATTTTT |
| | | | CCGATGCTTTTGATATCTGGGGCCACGGGACAATGGTCACCGTCTC |
| | | | TTCA |
| 440 | STIM005 | Amino acid sequence | QVQLVQSGAEVKKPGASVKVSCKASGYTFNSYGIIWVRQAPGQGLE |
| | -full | of STIM005 heavy | WMGWISVHNGNTNCAQKLQGRVTMTTDTSTSTAYMELRSLRTDDTA |
| | heavy | chain | VYYCARAGYDILTDFSDAFDIWGHGTMVTVSSASTKGPSVFPLAPS |
| | chain | | SKSTSGGTAALGCLVKDYFPEPVTVSWNSGALTSGVHTFPAVLQSS |
| | sequence | | GLYSLSSVVTVPSSSLGTQTYICNVNHKPSNTKVDKKVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYNSTYRVVSVLTVLHQDWLN |
| | | | GKEYKCKVSNKALPAPIEKTISKAKGQPREPQVYTLPPSRDELTKN |
| | | | QVSLTCLVKGFYPSDIAVEWESNGQPENNYKTTPPVLDSDGSFFLY |
| | | | SKLTVDKSRWQQGNVFSCSVMHEALHNHYTQKSLSLSPGK |
| 441 | STIM005 | Nucleic acid | CAGGTTCAGTTGGTGCAGTCTGGAGCTGAGGTGAAGAAGCCTGGGG |
| | -full | sequence of STIM005 | CCTCAGTGAAGGTCTCCTGCAAGGCTTCTGGTTACACCTTTAATAG |
| | heavy | heavy chain | TTATGGTATCATCTGGGTGCGACAGGCCCCTGGACAAGGGCTTGAG |
| | chain | | TGGATGGGATGGATCAGCGTTCACAATGGTAACACAAACTGTGCAC |
| | sequence | | AGAAGCTCCAGGGTAGAGTCACCATGACCACAGACACATCCACGAG |
| | | | CACAGCCTACATGGAGCTGAGGAGCCTGAGAACTGACGACACGGCC |
| | | | GTGTATTACTGTGCGAGAGCGGGTTACGATATTTTGACTGATTTTT |
| | | | CCGATGCTTTTGATATCTGGGGCCACGGGACAATGGTCACCGTCTC |
| | | | TTCA |
| | | | GCCAGCACCAAGGGCCCCTCTGTGTTCCCTCTGGCCCCTTCCAGCA |
| | | | AGTCCACCTCTGGCGGAACAGCCGCTCTGGGCTGCCTCGTGAAGGA |
| | | | CTACTTCCCCGAGCCTGTGACCGTGTCCTGGAACTCTGGCGCTCTG |
| | | | ACCAGCGGAGTGCACACCTTCCCTGCTGTGCTGCAGTCCTCCGGCC |
| | | | TGTACTCCCTGTCCTCCGTCGTGACCGTGCCTTCCAGCTCTCTGGG |
| | | | CACCCAGACCTACATCTGCAACGTGAACCACAAGCCCTCCAACACC |
| | | | AAGGTGGACAAGAAGGTGGAACCCAAGTCCTGCGACAAGACCCACA |
| | | | CCTGTCCCCCTTGTCCTGCCCCTGAACTGCTGGGCGGACCTTCCGT |
| | | | GTTCCTGTTCCCCCCAAAGCCCAAGGACACCCTGATGATCTCCCGG |
| | | | ACCCCCGAAGTGACCTGCGTGGTGGTGGATGTGTCCCACGAGGACC |
| | | | CTGAAGTGAAGTTCAATTGGTACGTGGACGGCGTGGAAGTGCACAA |
| | | | CGCCAAGACCAAGCCTAGAGAGGAACAGTACAACTCCACCTACCGG |
| | | | GTGGTGTCCGTGCTGACCGTGCTGCACCAGGATTGGCTGAACGGCA |
| | | | AAGAGTACAAGTGCAAGGTGTCCAACAAGGCCCTGCCTGCCCCCAT |
| | | | CGAAAAGACCATCTCCAAGGCCAAGGGCCAGCCCCGGGAACCCCAG |
| | | | GTGTACACACTGCCCCCTAGCAGGGACGAGCTGACCAAGAACCAGG |
| | | | TGTCCCTGACCTGTCTCGTGAAAGGCTTCTACCCCTCCGATATCGC |
| | | | CGTGGAATGGGAGTCCAACGGCCAGCCTGAGAACAACTACAAGACC |
| | | | ACCCCCCCTGTGCTGGACTCCGACGGCTCATTCTTCCTGTACAGCA |
| | | | AGCTGACAGTGGACAAGTCCCGGTGGCAGCAGGGCAACGTGTTCTC |
| | | | CTGCTCCGTGATGCACGAGGCCCTGCACAACCACTACACCCAGAAG |
| | | | TCCCTGTCCCTGAGCCCCGGCAAGTGATGA |
| 442 | STIM005 | Amino acid sequence | QNINNF |
| | -CDRL1 | of CDRL1 of STIM005 | |
| | | using IMGT | |
| 443 | STIM005 | Amino acid sequence | AAS |
| | -CDRL2 | of CDRL2 of STIM005 | |
| | | using IMGT | |
| 444 | STIM005 | Amino acid sequence | QQSYGIPW |
| | -CDRL3 | of CDRL3 of STIM005 | |
| | | using IMGT | |
| 445 | STIM005 | Amino acid sequence | DIQMTQSPSSLSASVGDRVTITCRASQNINNFLNWYQQKEGKGPKL |
| | -Light | of V_{L} of STIM005 | LIYAASSLQRGIPSTFSGSGSGTDFTLTISSLQPEDFATYICQQSY |
| | chain | | GIPWVGQGTKVEIK |
| | variable | | |
| | region | | |
| 446 | STIM005 | Nucleic acid | GACATCCAGATGACCCAGTCTCCATCCTCCCTGTCTGCATCTGTAG |
| | -Light | sequence of V_{L} of | GAGACAGAGTCACCATCACTTGCCGGGCAAGTCAGAACATTAATAA |
| | chain | STIM005 | CTTTTTAAATTGGTATCAGCAGAAAGAAGGGAAAGGCCCTAAGCTC |
| | variable | | CTGATCTATGCAGCATCCAGTTTGCAAAGAGGGATACCATCAACGT |
| | region | | TCAGTGGCAGTGGATCTGGGACAGACTTCACTCTCACCATCAGCAG |
| | | | TCTGCAACCTGAAGATTTTGCAACTTACATCTGTCAACAGAGCTAC |
| | | | GGTATCCCGTGGGTCGGCCAAGGGACCAAGGTGGAAATCAAA |
| 447 | STIM005 | Amino acid sequence | DIQMTQSPSSLSASVGDRVTITCRASQNINNFLNWYQQKEGKGPKL |
| | -full | of STIM005 light | LIYAASSLQRGIPSTFSGSGSGTDFTLTISSLQPEDFATYICQQSY |
| | light | chain | GIPWVGQGTKVEIK |
| | chain | | RTVAAPSVFIFPPSDEQLKSGTASVVCLLNNFYPREAKVQWKVDNA |
| | sequence | | LQSGNSQESVTEQDSKDSTYSLSSTLTLSKADYEKHKVYACEVTHQ |
| | | | GLSSPVTKSENRGEC |
| 448 | STIM005 | Nucleic acid | GACATCCAGATGACCCAGTCTCCATCCTCCCTGTCTGCATCTGTAG |
| | -full | sequence of STIM005 | GAGACAGAGTCACCATCACTTGCCGGGCAAGTCAGAACATTAATAA |
| | light | light chain | CTTTTTAAATTGGTATCAGCAGAAAGAAGGGAAAGGCCCTAAGCTC |
| | chain | | CTGATCTATGCAGCATCCAGTTTGCAAAGAGGGATACCATCAACGT |
| | sequence | | TCAGTGGCAGTGGATCTGGGACAGACTTCACTCTCACCATCAGCAG |
| | | | TCTGCAACCTGAAGATTTTGCAACTTACATCTGTCAACAGAGCTAC |
| | | | GGTATCCCGTGGGTCGGCCAAGGGACCAAGGTGGAAATCAAAcgta |
| | | | cggtggccgctccctccgtgttcatcttcccaccttccgacgagca |
| | | | gctgaagtccggcaccgcttctgtcgtgtgcctgctgaacaacttc |
| | | | tacccccgcgaggccaaggtgcagtggaaggtggacaacgccctgc |
| | | | agtccggcaactcccaggaatccgtgaccgagcaggactccaagga |
| | | | cagcacctactccctgtcctccaccctgaccctgtccaaggccgac |
| | | | tacgagaagcacaaggtgtacgcctgcgaagtgacccaccagggcc |
| | | | tgtctagccccgtgaccaagtctttcaaccggggcgagtgt |
| 449 | STIM006 | Amino acid sequence | GFTFSDYE |
| | -CDRH1 | of CDRH1 of STIM006 | |
| | | using IMGT | |
| 450 | STIM006 | Amino acid sequence | ISSSGSTI |
| | -CDRH2 | of CDRH2 of STIM006 | |
| | | using IMGT | |
| 451 | STIM006 | Amino acid sequence | ARDHYDGSGIYPLYYYYGLDV |
| | -CDRH3 | of CDRH3 of STIM006 | |
| | | using IMGT | |
| 452 | STIM006 | Amino acid sequence | QVQLVESGGGLVKPGGSLRLSCAASGFTFSDYFMSWIRQAPGKGLE |
| | -Heavy | of V_{H} of STIM006 | WISYISSSGSTIYYADSVRGRFTISRDNAKYSLYLQMNSLRSEDTA |
| | chain | | VYYCARDHYDGSGIYPLYYYYGLDVWGQGTTVTVSS |
| | variable | | |
| | region | | |
| 453 | STIM006 | Nucleic acid | CAGGTGCAGCTGGTGGAGTCTGGGGGAGGCTTGGTCAAGCCTGGAG |
| | -Heavy | sequence of V_{H} of | GGTCCCTGAGACTCTCCTGTGCAGCCTCTGGATTCACCTTCAGTGA |
| | chain | STIM006 | CTACTTCATGAGCTGGATCCGCCAGGCGCCAGGGAAGGGGCTGGAG |
| | variable | | TGGATTTCATACATTAGTTCTAGTGGTAGTACCATATACTACGCAG |
| | region | | ACTCTGTGAGGGGCCGATTCACCATCTCCAGGGACAACGCCAAGTA |
| | | | CTCACTGTATCTGCAAATGAACAGCCTGAGATCCGAGGACACGGCC |
| | | | GTGTATTACTGTGCGAGAGATCACTACGATGGTTCGGGGATTTATC |
| | | | CCCTCTACTACTATTACGGTTTGGACGTCTGGGGCCAGGGGACCAC |
| | | | GGTCACCGTCTCCTCA |
| 454 | STIM006 | Amino acid sequence | QVQLVESGGGLVKPGGSLRLSCAASGFTFSDYFMSWIRQAPGKGLE |
| | -full | of STIM006 heavy | WISYISSSGSTIYYADSVRGRFTISRDNAKYSLYLQMNSLRSEDTA |
| | heavy | chain | VYYCARDHYDGSGIYPLYYYYGLDVWGQGTTVTVSSASTKGPSVFP |
| | chain | | LAPSSKSTSGGTAALGCLVKDYFPEPVTVSWNSGALTSGVHTFPAV |
| | sequence | | LQSSGLYSLSSVVTVPSSSLGTQTYICNVNHKPSNTKVDKKVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYNSTYRVVSVLTVLHQ |
| | | | DWLNGKEYKCKVSNKALPAPIEKTISKAKGQPREPQVYTLPPSRDE |
| | | | LTKNQVSLTCLVKGFYPSDIAVEWESNGQPENNYKTTPPVLDSDGS |
| | | | FFLYSKLTVDKSRWQQGNVFSCSVMHEALHNHYTQKSLSLSPGK |
| 455 | STIM006 | Nucleic acid | CAGGTGCAGCTGGTGGAGTCTGGGGGAGGCTTGGTCAAGCCTGGAG |
| | -full | sequence of STIM006 | GGTCCCTGAGACTCTCCTGTGCAGCCTCTGGATTCACCTTCAGTGA |
| | heavy | heavy chain | CTACTTCATGAGCTGGATCCGCCAGGCGCCAGGGAAGGGGCTGGAG |
| | chain | | TGGATTTCATACATTAGTTCTAGTGGTAGTACCATATACTACGCAG |
| | sequence | | ACTCTGTGAGGGGCCGATTCACCATCTCCAGGGACAACGCCAAGTA |
| | | | CTCACTGTATCTGCAAATGAACAGCCTGAGATCCGAGGACACGGCC |
| | | | GTGTATTACTGTGCGAGAGATCACTACGATGGTTCGGGGATTTATC |
| | | | CCCTCTACTACTATTACGGTTTGGACGTCTGGGGCCAGGGGACCAC |
| | | | GGTCACCGTCTCCTCAGCCAGCACCAAGGGCCCCTCTGTGTTCCCT |
| | | | CTGGCCCCTTCCAGCAAGTCCACCTCTGGCGGAACAGCCGCTCTGG |
| | | | GCTGCCTCGTGAAGGACTACTTCCCCGAGCCTGTGACCGTGTCCTG |
| | | | GAACTCTGGCGCTCTGACCAGCGGAGTGCACACCTTCCCTGCTGTG |
| | | | CTGCAGTCCTCCGGCCTGTACTCCCTGTCCTCCGTCGTGACCGTGC |
| | | | CTTCCAGCTCTCTGGGCACCCAGACCTACATCTGCAACGTGAACCA |
| | | | CAAGCCCTCCAACACCAAGGTGGACAAGAAGGTGGAACCCAAGTCC |
| | | | TGCGACAAGACCCACACCTGTCCCCCTTGTCCTGCCCCTGAACTGC |
| | | | TGGGCGGACCTTCCGTGTTCCTGTTCCCCCCAAAGCCCAAGGACAC |
| | | | CCTGATGATCTCCCGGACCCCCGAAGTGACCTGCGTGGTGGTGGAT |
| | | | GTGTCCCACGAGGACCCTGAAGTGAAGTTCAATTGGTACGTGGACG |
| | | | GCGTGGAAGTGCACAACGCCAAGACCAAGCCTAGAGAGGAACAGTA |
| | | | CAACTCCACCTACCGGGTGGTGTCCGTGCTGACCGTGCTGCACCAG |
| | | | GATTGGCTGAACGGCAAAGAGTACAAGTGCAAGGTGTCCAACAAGG |
| | | | CCCTGCCTGCCCCCATCGAAAAGACCATCTCCAAGGCCAAGGGCCA |
| | | | GCCCCGGGAACCCCAGGTGTACACACTGCCCCCTAGCAGGGACGAG |
| | | | CTGACCAAGAACCAGGTGTCCCTGACCTGTCTCGTGAAAGGCTTCT |
| | | | ACCCCTCCGATATCGCCGTGGAATGGGAGTCCAACGGCCAGCCTGA |
| | | | GAACAACTACAAGACCACCCCCCCTGTGCTGGACTCCGACGGCTCA |
| | | | TTCTTCCTGTACAGCAAGCTGACAGTGGACAAGTCCCGGTGGCAGC |
| | | | AGGGCAACGTGTTCTCCTGCTCCGTGATGCACGAGGCCCTGCACAA |
| | | | CCACTACACCCAGAAGTCCCTGTCCCTGAGCCCCGGCAAGTGATGA |
| 456 | STIM006 | Amino acid sequence | QSLLHSNGYNY |
| | -CDRL1 | of CDRL1 of STIM006 | |
| | | using IMGT | |
| 457 | STIM006 | Amino acid sequence | LGS |
| | -CDRL2 | of CDRL2 of STIM006 | |
| | | using IMGT | |
| 458 | STIM006 | Amino acid sequence | MQALQTPRS |
| | -CDRL3 | of CDRL3 of STIM006 | |
| | | using IMGT | |
| 459 | STIM006 | Amino acid sequence | IVMTQSPLSLPVTPGEPASISCRSSQSLLHSNGYNYLDYYLQKPGQ |
| | -Light | of V_{L} of STIM006 | SPQLLIYLGSYRASGVPDRFSGSGSGTDFTLKISRVEAEDVGVYYC |
| | chain | | MQALQTPRSFGQGTTLEIK |
| | variable | | |
| | region | | |
| 460 | STIM006 | Nucleic acid | ATTGTGATGACTCAGTCTCCACTCTCCCTACCCGTCACCCCTGGAG |
| | -Light | sequence of V_{L} of | AGCCGGCCTCCATCTCCTGCAGGTCTAGTCAGAGCCTCCTGCATAG |
| | chain | STIM006 | TAATGGATACAACTATTTGGATTATTACCTGCAGAAGCCAGGGCAG |
| | variable | | TCTCCACAGCTCCTGATCTATTTGGGTTCTTATCGGGCCTCCGGGG |
| | region | | TCCCTGACAGGTTCAGTGGCAGTGGATCAGGCACAGATTTTACACT |
| | | | GAAAATCAGCAGAGTGGAGGCTGAGGATGTTGGGGTTTATTACTGC |
| | | | ATGCAAGCTCTACAAACTCCTCGCAGTTTTGGCCAGGGGACCACGC |
| | | | TGGAGATCAAA |
| 461 | STIM006 | Amino acid sequence | IVMTQSPLSLPVTPGEPASISCRSSQSLLHSNGYNYLDYYLQKPGQ |
| | -full | of STIM006 light | SPQLLIYLGSYRASGVPDRFSGSGSGTDFTLKISRVEAEDVGVYYC |
| | light | chain | MQALQTPRSFGQGTTLEIKRTVAAPSVFIFPPSDEQLKSGTASVVC |
| | chain | | LLNNFYPREAKVQWKVDNALQSGNSQESVTEQDSKDSTYSLSSTLT |
| | sequence | | LSKADYEKHKVYACEVTHQGLSSPVTKSENRGEC |
| 462 | STIM006 | Nucleic acid | ATTGTGATGACTCAGTCTCCACTCTCCCTACCCGTCACCCCTGGAG |
| | -full | sequence of STIM006 | AGCCGGCCTCCATCTCCTGCAGGTCTAGTCAGAGCCTCCTGCATAG |
| | light | light chain | TAATGGATACAACTATTTGGATTATTACCTGCAGAAGCCAGGGCAG |
| | chain | | TCTCCACAGCTCCTGATCTATTTGGGTTCTTATCGGGCCTCCGGGG |
| | sequence | | TCCCTGACAGGTTCAGTGGCAGTGGATCAGGCACAGATTTTACACT |
| | | | GAAAATCAGCAGAGTGGAGGCTGAGGATGTTGGGGTTTATTACTGC |
| | | | ATGCAAGCTCTACAAACTCCTCGCAGTTTTGGCCAGGGGACCACGC |
| | | | TGGAGATCAAAcgtacggtggccgctccctccgtgttcatcttccc |
| | | | accttccgacgagcagctgaagtccggcaccgcttctgtcgtgtgc |
| | | | ctgctgaacaacttctacccccgcgaggccaaggtgcagtggaagg |
| | | | tggacaacgccctgcagtccggcaactcccaggaatccgtgaccga |
| | | | gcaggactccaaggacagcacctactccctgtcctccaccctgacc |
| | | | ctgtccaaggccgactacgagaagcacaaggtgtacgcctgcgaag |
| | | | tgacccaccagggcctgtctagccccgtgaccaagtctttcaaccg |
| | | | gggcgagtgt |
| 463 | STIM007 | Amino acid sequence | GFSLSTTGVG |
| | -CDRH1 | of CDRH1 of STIM007 | |
| | | using IMGT | |
| 464 | STIM007 | Amino acid sequence | IYWDDDK |
| | -CDRH2 | of CDRH2 of STIM007 | |
| | | using IMGT | |
| 465 | STIM007 | Amino acid sequence | THGYGSASYYHYGMDV |
| | -CDRH3 | of CDRH3 of STIM007 | |
| | | using IMGT | |
| 466 | STIM007 | Amino acid sequence | QITLKESGPTLVKPTQTLTLTCTFSGFSLSTTGVGVGWIRQPPGKA |
| | -Heavy | of V_{H} of STIM007 | LEWLAVIYWDDDKRYSPSLKSRLTITKDTSKNQVVLTMTNMDPVDT |
| | chain | | ATYFCTHGYGSASYYHYGMDVWGQGTTVTVSS |
| | variable | | |
| | region | | |
| 467 | STIM007 | Nucleic acid | CAGATCACCTTGAAGGAGTCTGGTCCTACGCTGGTGAAACCCACAC |
| | -Heavy | sequence of V_{H} of | AGACCCTCACGCTGACCTGCACCTTCTCTGGGTTCTCACTCAGCAC |
| | chain | STIM007 | TACTGGAGTGGGTGTGGGCTGGATCCGTCAGCCCCCAGGAAAGGCC |
| | variable | | CTGGAGTGGCTTGCAGTCATTTATTGGGATGATGATAAGCGCTACA |
| | region | | GCCCATCTCTGAAGAGCAGACTCACCATCACCAAGGACACCTCCAA |
| | | | AAACCAGGTGGTCCTTACAATGACCAACATGGACCCTGTGGACACA |
| | | | GCCACATATTTCTGTACACACGGATATGGTTCGGCGAGTTATTACC |
| | | | ACTACGGTATGGACGTCTGGGGCCAAGGGACCACGGTCACCGTCTC |
| | | | CTCA |
| 468 | STIM007 | Amino acid sequence | QITLKESGPTLVKPTQTLTLTCTFSGFSLSTTGVGVGWIRQPPGKA |
| | -full | of STIM007 heavy | LEWLAVIYWDDDKRYSPSLKSRLTITKDTSKNQVVLTMTNMDPVDT |
| | heavy | chain | ATYFCTHGYGSASYYHYGMDVWGQGTTVTVSSASTKGPSVFPLAPS |
| | chain | | SKSTSGGTAALGCLVKDYFPEPVTVSWNSGALTSGVHTFPAVLQSS |
| | sequence | | GLYSLSSVVTVPSSSLGTQTYICNVNHKPSNTKVDKKVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYNSTYRVVSVLTVLHQDWLN |
| | | | GKEYKCKVSNKALPAPIEKTISKAKGQPREPQVYTLPPSRDELTKN |
| | | | QVSLTCLVKGFYPSDIAVEWESNGQPENNYKTTPPVLDSDGSFFLY |
| | | | SKLTVDKSRWQQGNVFSCSVMHEALHNHYTQKSLSLSPGK |
| 469 | STIM007 | Nucleic acid | CAGATCACCTTGAAGGAGTCTGGTCCTACGCTGGTGAAACCCACAC |
| | -full | sequence of STIM007 | AGACCCTCACGCTGACCTGCACCTTCTCTGGGTTCTCACTCAGCAC |
| | heavy | heavy chain | TACTGGAGTGGGTGTGGGCTGGATCCGTCAGCCCCCAGGAAAGGCC |
| | chain | | CTGGAGTGGCTTGCAGTCATTTATTGGGATGATGATAAGCGCTACA |
| | sequence | | GCCCATCTCTGAAGAGCAGACTCACCATCACCAAGGACACCTCCAA |
| | | | AAACCAGGTGGTCCTTACAATGACCAACATGGACCCTGTGGACACA |
| | | | GCCACATATTTCTGTACACACGGATATGGTTCGGCGAGTTATTACC |
| | | | ACTACGGTATGGACGTCTGGGGCCAAGGGACCACGGTCACCGTCTC |
| | | | CTCA |
| | | | GCCAGCACCAAGGGCCCCTCTGTGTTCCCTCTGGCCCCTTCCAGCA |
| | | | AGTCCACCTCTGGCGGAACAGCCGCTCTGGGCTGCCTCGTGAAGGA |
| | | | CTACTTCCCCGAGCCTGTGACCGTGTCCTGGAACTCTGGCGCTCTG |
| | | | ACCAGCGGAGTGCACACCTTCCCTGCTGTGCTGCAGTCCTCCGGCC |
| | | | TGTACTCCCTGTCCTCCGTCGTGACCGTGCCTTCCAGCTCTCTGGG |
| | | | CACCCAGACCTACATCTGCAACGTGAACCACAAGCCCTCCAACACC |
| | | | AAGGTGGACAAGAAGGTGGAACCCAAGTCCTGCGACAAGACCCACA |
| | | | CCTGTCCCCCTTGTCCTGCCCCTGAACTGCTGGGCGGACCTTCCGT |
| | | | GTTCCTGTTCCCCCCAAAGCCCAAGGACACCCTGATGATCTCCCGG |
| | | | ACCCCCGAAGTGACCTGCGTGGTGGTGGATGTGTCCCACGAGGACC |
| | | | CTGAAGTGAAGTTCAATTGGTACGTGGACGGCGTGGAAGTGCACAA |
| | | | CGCCAAGACCAAGCCTAGAGAGGAACAGTACAACTCCACCTACCGG |
| | | | GTGGTGTCCGTGCTGACCGTGCTGCACCAGGATTGGCTGAACGGCA |
| | | | AAGAGTACAAGTGCAAGGTGTCCAACAAGGCCCTGCCTGCCCCCAT |
| | | | CGAAAAGACCATCTCCAAGGCCAAGGGCCAGCCCCGGGAACCCCAG |
| | | | GTGTACACACTGCCCCCTAGCAGGGACGAGCTGACCAAGAACCAGG |
| | | | TGTCCCTGACCTGTCTCGTGAAAGGCTTCTACCCCTCCGATATCGC |
| | | | CGTGGAATGGGAGTCCAACGGCCAGCCTGAGAACAACTACAAGACC |
| | | | ACCCCCCCTGTGCTGGACTCCGACGGCTCATTCTTCCTGTACAGCA |
| | | | AGCTGACAGTGGACAAGTCCCGGTGGCAGCAGGGCAACGTGTTCTC |
| | | | CTGCTCCGTGATGCACGAGGCCCTGCACAACCACTACACCCAGAAG |
| | | | TCCCTGTCCCTGAGCCCCGGCAAGTGATGA |
| 470 | STIM007- | Amino acid sequence | QSVTNY |
| | CDRL1 | of CDRL1 of STIM007 | |
| | | using IMGT | |
| 471 | STIM007- | Amino acid sequence | DAS |
| | CDRL2 | of CDRL2 of STIM007 | |
| | | using IMGT | |
| 472 | STIM007- | Amino acid sequence | QHRSNWPLT |
| | CDRL3 | of CDRL3 of STIM007 | |
| | | using IMGT | |
| 473 | STIM007 | Amino acid sequence | EIVLTQSPATLSLSPGERATLSCRASQSVTNYLAWHQQKPGQAPRL |
| | -Light | of V_{L} of STIM007 | LIYDASNRATGIPARFSGSGSGTDFTLTISSLEPEDFAVYYCQHRS |
| | chain | | NWPLTFGGGTKVEIK |
| | variable | | |
| | region | | |
| 474 | STIM007 | Nucleic acid | GAAATTGTATTGACACAGTCTCCAGCCACCCTGTCTTTGTCTCCAG |
| | -Light | sequence of V_{L} of | GGGAAAGAGCCACCCTCTCCTGCAGGGCCAGTCAGAGTGTTACCAA |
| | chain | STIM007 | CTACTTAGCCTGGCACCAACAGAAACCTGGCCAGGCTCCCAGGCTC |
| | variable | | CTCATCTATGATGCATCCAACAGGGCCACTGGCATCCCAGCCAGGT |
| | region | | TCAGTGGCAGTGGGTCTGGGACAGACTTCACTCTCACCATCAGCAG |
| | | | CCTAGAGCCTGAAGATTTTGCAGTTTATTACTGTCAGCACCGTAGC |
| | | | AACTGGCCTCTCACTTTCGGCGGAGGGACCAAGGTGGAGATCAAAC |
| 475 | STIM007 | Amino acid sequence | EIVLTQSPATLSLSPGERATLSCRASQSVTNYLAWHQQKPGQAPRL |
| | -full | of STIM007 light | LIYDASNRATGIPARFSGSGSGTDFTLTISSLEPEDFAVYYCQHRS |
| | light | chain | NWPLTFGGGTKVEIKRTVAAPSVFIFPPSDEQLKSGTASVVCLLNN |
| | chain | | FYPREAKVQWKVDNALQSGNSQESVTEQDSKDSTYSLSSTLTLSKA |
| | sequence | | DYEKHKVYACEVTHQGLSSPVTKSENRGEC |
| 476 | STIM007 | Nucleic acid | GAAATTGTATTGACACAGTCTCCAGCCACCCTGTCTTTGTCTCCAG |
| | -full | sequence of STIM007 | GGGAAAGAGCCACCCTCTCCTGCAGGGCCAGTCAGAGTGTTACCAA |
| | light | light chain | CTACTTAGCCTGGCACCAACAGAAACCTGGCCAGGCTCCCAGGCTC |
| | chain | | CTCATCTATGATGCATCCAACAGGGCCACTGGCATCCCAGCCAGGT |
| | sequence | | TCAGTGGCAGTGGGTCTGGGACAGACTTCACTCTCACCATCAGCAG |
| | | | CCTAGAGCCTGAAGATTTTGCAGTTTATTACTGTCAGCACCGTAGC |
| | | | AACTGGCCTCTCACTTTCGGCGGAGGGACCAAGGTGGAGATCAAAC |
| | | | cgtacggtggccgctccctccgtgttcatcttcccaccttccgacg |
| | | | agcagctgaagtccggcaccgcttctgtcgtgtgcctgctgaacaa |
| | | | cttctacccccgcgaggccaaggtgcagtggaaggtggacaacgcc |
| | | | ctgcagtccggcaactcccaggaatccgtgaccgagcaggactcca |
| | | | aggacagcacctactccctgtcctccaccctgaccctgtccaaggc |
| | | | cgactacgagaagcacaaggtgtacgcctgcgaagtgacccaccag |
| | | | ggcctgtctagccccgtgaccaagtotttcaaccggggcgagtgt |
| 477 | STIM008- | Amino acid sequence | GFSLSTSGVG |
| | CDRH1 | of CDRH1 of STIM008 | |
| | | using IMGT | |
| 478 | STIM008- | Amino acid sequence | IYWDDDK |
| | CDRH2 | of CDRH2 of STIM008 | |
| | | using IMGT | |
| 479 | STIM008- | Amino acid sequence | THGYGSASYYHYGMDV |
| | CDRH3 | of CDRH3 of STIM008 | |
| | | using IMGT | |
| 480 | STIM008 | Amino acid sequence | QITLKESGPTLVKPTQTLTLTCTFSGFSLSTSGVGVGWIRQPPGKA |
| | -Heavy | of V_{H} of STIM008 | LEWLAVIYWDDDKRYSPSLKSRLTITKDTSKNQVVLTMTNMDPVDT |
| | chain | | ATYFCTHGYGSASYYHYGMDVWGQGTTVTVSS |
| | variable | | |
| | region | | |
| 481 | STIM008 | Nucleic acid | CAGATCACCTTGAAGGAGTCTGGTCCTACGCTGGTGAAACCCACAC |
| | -Heavy | sequence of V_{H} of | AGACCCTCACGCTGACCTGCACCTTCTCTGGGTTCTCACTCAGCAC |
| | chain | STIM008 | TAGTGGAGTGGGTGTGGGCTGGATCCGTCAGCCCCCAGGAAAGGCC |
| | variable | | CTGGAGTGGCTTGCAGTCATTTATTGGGATGATGATAAGCGCTACA |
| | region | | GCCCATCTCTGAAGAGCAGGCTCACCATCACCAAGGACACCTCCAA |
| | | | AAACCAGGTGGTCCTTACAATGACCAACATGGACCCTGTGGACACA |
| | | | GCCACATATTTCTGTACACACGGATATGGTTCGGCGAGTTATTACC |
| | | | ACTACGGTATGGACGTCTGGGGCCAAGGGACCACGGTCACCGTCTC |
| | | | CTCA |
| 482 | STIM008 | Amino acid sequence | QITLKESGPTLVKPTQTLTLTCTFSGFSLSTSGVGVGWIRQPPGKA |
| | -full | of STIM008 heavy | LEWLAVIYWDDDKRYSPSLKSRLTITKDTSKNQVVLTMTNMDPVDT |
| | heavy | chain | ATYFCTHGYGSASYYHYGMDVWGQGTTVTVSSASTKGPSVFPLAPS |
| | chain | | SKSTSGGTAALGCLVKDYFPEPVTVSWNSGALTSGVHTFPAVLQSS |
| | sequence | | GLYSLSSVVTVPSSSLGTQTYICNVNHKPSNTKVDKKVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYNSTYRVVSVLTVLHQDWLN |
| | | | GKEYKCKVSNKALPAPIEKTISKAKGQPREPQVYTLPPSRDELTKN |
| | | | QVSLTCLVKGFYPSDIAVEWESNGQPENNYKTTPPVLDSDGSFFLY |
| | | | SKLTVDKSRWQQGNVFSCSVMHEALHNHYTQKSLSLSPGK |
| 483 | STIM008 | Nucleic acid | CAGATCACCTTGAAGGAGTCTGGTCCTACGCTGGTGAAACCCACAC |
| | -full | sequence of STIM008 | AGACCCTCACGCTGACCTGCACCTTCTCTGGGTTCTCACTCAGCAC |
| | heavy | heavy chain | TAGTGGAGTGGGTGTGGGCTGGATCCGTCAGCCCCCAGGAAAGGCC |
| | chain | | CTGGAGTGGCTTGCAGTCATTTATTGGGATGATGATAAGCGCTACA |
| | sequence | | GCCCATCTCTGAAGAGCAGGCTCACCATCACCAAGGACACCTCCAA |
| | | | AAACCAGGTGGTCCTTACAATGACCAACATGGACCCTGTGGACACA |
| | | | GCCACATATTTCTGTACACACGGATATGGTTCGGCGAGTTATTACC |
| | | | ACTACGGTATGGACGTCTGGGGCCAAGGGACCACGGTCACCGTCTC |
| | | | CTCAGCCAGCACCAAGGGCCCCTCTGTGTTCCCTCTGGCCCCTTCC |
| | | | AGCAAGTCCACCTCTGGCGGAACAGCCGCTCTGGGCTGCCTCGTGA |
| | | | AGGACTACTTCCCCGAGCCTGTGACCGTGTCCTGGAACTCTGGCGC |
| | | | TCTGACCAGCGGAGTGCACACCTTCCCTGCTGTGCTGCAGTCCTCC |
| | | | GGCCTGTACTCCCTGTCCTCCGTCGTGACCGTGCCTTCCAGCTCTC |
| | | | TGGGCACCCAGACCTACATCTGCAACGTGAACCACAAGCCCTCCAA |
| | | | CACCAAGGTGGACAAGAAGGTGGAACCCAAGTCCTGCGACAAGACC |
| | | | CACACCTGTCCCCCTTGTCCTGCCCCTGAACTGCTGGGCGGACCTT |
| | | | CCGTGTTCCTGTTCCCCCCAAAGCCCAAGGACACCCTGATGATCTC |
| | | | CCGGACCCCCGAAGTGACCTGCGTGGTGGTGGATGTGTCCCACGAG |
| | | | GACCCTGAAGTGAAGTTCAATTGGTACGTGGACGGCGTGGAAGTGC |
| | | | ACAACGCCAAGACCAAGCCTAGAGAGGAACAGTACAACTCCACCTA |
| | | | CCGGGTGGTGTCCGTGCTGACCGTGCTGCACCAGGATTGGCTGAAC |
| | | | GGCAAAGAGTACAAGTGCAAGGTGTCCAACAAGGCCCTGCCTGCCC |
| | | | CCATCGAAAAGACCATCTCCAAGGCCAAGGGCCAGCCCCGGGAACC |
| | | | CCAGGTGTACACACTGCCCCCTAGCAGGGACGAGCTGACCAAGAAC |
| | | | CAGGTGTCCCTGACCTGTCTCGTGAAAGGCTTCTACCCCTCCGATA |
| | | | TCGCCGTGGAATGGGAGTCCAACGGCCAGCCTGAGAACAACTACAA |
| | | | GACCACCCCCCCTGTGCTGGACTCCGACGGCTCATTCTTCCTGTAC |
| | | | AGCAAGCTGACAGTGGACAAGTCCCGGTGGCAGCAGGGCAACGTGT |
| | | | TCTCCTGCTCCGTGATGCACGAGGCCCTGCACAACCACTACACCCA |
| | | | GAAGTCCCTGTCCCTGAGCCCCGGCAAGTGATGA |
| 484 | STIM008- | Amino acid sequence | QSVTNY |
| | CDRL1 | of CDRL1 of STIM008 | |
| | | using IMGT | |
| 485 | STIM008- | Amino acid sequence | DAS |
| | CDRL2 | of CDRL2 of STIM008 | |
| | | using IMGT | |
| 486 | STIM008- | Amino acid sequence | QQRSNWPLT |
| | CDRL3 | of CDRL3 of STIM008 | |
| | | using IMGT | |
| 487 | STIM008 | Amino acid sequence | EIVLTQSPATLSLSPGERATLSCRASQSVTNYLAWHQQKPGQAPRL |
| | -Light | of V_{L} of STIM008 | LIYDASNRATGIPARFSGSGSGTDFTLTISSLEPEDFAVYYCQQRS |
| | chain | | NWPLTFGGGTKVEIK |
| | variable | | |
| | region | | |
| 488 | STIM008 | Nucleic acid | GAAATTGTGTTGACACAGTCTCCAGCCACCCTGTCTTTGTCTCCAG |
| | -Light | sequence of V_{L} of | GGGAAAGAGCCACCCTCTCCTGCAGGGCCAGTCAGAGTGTTACCAA |
| | chain | STIM008 | CTACTTAGCCTGGCACCAACAGAAACCTGGCCAGGCTCCCAGGCTC |
| | variable | | CTCATCTATGATGCATCCAACAGGGCCACTGGCATCCCAGCCAGGT |
| | region | | TCAGTGGCAGTGGGTCTGGGACAGACTTCACTCTCACCATCAGCAG |
| | | | CCTAGAGCCTGAAGATTTTGCAGTTTATTACTGTCAGCAGCGTAGC |
| | | | AACTGGCCTCTCACTTTCGGCGGAGGGACCAAGGTGGAGATCAAA |
| 489 | STIM008 | Amino acid sequence | EIVLTQSPATLSLSPGERATLSCRASQSVTNYLAWHQQKPGQAPRL |
| | -full | of STIM008 light | LIYDASNRATGIPARFSGSGSGTDFTLTISSLEPEDFAVYYCQQRS |
| | light | chain | NWPLTFGGGTKVEIKRTVAAPSVFIFPPSDEQLKSGTASVVCLLNN |
| | chain | | FYPREAKVQWKVDNALQSGNSQESVTEQDSKDSTYSLSSTLTLSKA |
| | sequence | | DYEKHKVYACEVTHQGLSSPVTKSENRGEC |
| 490 | STIM008 | Nucleic acid | GAAATTGTGTTGACACAGTCTCCAGCCACCCTGTCTTTGTCTCCAG |
| | -full | sequence of STIM008 | GGGAAAGAGCCACCCTCTCCTGCAGGGCCAGTCAGAGTGTTACCAA |
| | light | light chain | CTACTTAGCCTGGCACCAACAGAAACCTGGCCAGGCTCCCAGGCTC |
| | chain | | CTCATCTATGATGCATCCAACAGGGCCACTGGCATCCCAGCCAGGT |
| | sequence | | TCAGTGGCAGTGGGTCTGGGACAGACTTCACTCTCACCATCAGCAG |
| | | | CCTAGAGCCTGAAGATTTTGCAGTTTATTACTGTCAGCAGCGTAGC |
| | | | AACTGGCCTCTCACTTTCGGCGGAGGGACCAAGGTGGAGATCAAAc |
| | | | gtacggtggccgctccctccgtgttcatcttcccaccttccgacga |
| | | | gcagctgaagtccggcaccgcttctgtcgtgtgcctgctgaacaac |
| | | | ttctacccccgcgaggccaaggtgcagtggaaggtggacaacgccc |
| | | | tgcagtccggcaactcccaggaatccgtgaccgagcaggactccaa |
| | | | ggacagcacctactccctgtcctccaccctgaccctgtccaaggcc |
| | | | gactacgagaagcacaaggtgtacgcctgcgaagtgacccaccagg |
| | | | gcctgtctagccccgtgaccaagtotttcaaccggggcgagtgt |
| 491 | STIM009- | Amino acid sequence | GFTFSDYY |
| | CDRH1 | of CDRH1 of STIM009 | |
| | | using IMGT | |
| 492 | STIM009- | Amino acid sequence | ISSSGSTI |
| | CDRH2 | of CDRH2 of STIM009 | |
| | | using IMGT | |
| 493 | STIM009- | Amino acid sequence | ARDFYDILTDSPYFYYGVDV |
| | CDRH3 | of CDRH3 of STIM009 | |
| | | using IMGT | |
| 494 | STIM009 | Amino acid sequence | QVQLVESGGGLVKPGGSLRLSCAASGFTFSDYYMSWIRQAPGKGLE |
| | -Heavy | of V_{H} of STIM009 | WVSYISSSGSTIYYADSVKGRFTISRDNAKNSLYLQINSLRAEDTA |
| | chain | | VYYCARDFYDILTDSPYFYYGVDVWGQGTTVTVSS |
| | variable | | |
| | region | | |
| 495 | STIM009 | Nucleic acid | CAGGTGCAGCTGGTGGAGTCTGGGGGAGGCTTGGTCAAGCCTGGAG |
| | -Heavy | sequence of V_{H} of | GGTCCCTGAGACTCTCCTGTGCAGCCTCTGGATTCACCTTCAGTGA |
| | chain | STIM009 | CTACTACATGAGCTGGATCCGCCAGGCTCCAGGGAAGGGGCTGGAG |
| | variable | | TGGGTTTCATACATTAGTAGTAGTGGTAGTACCATATACTACGCAG |
| | region | | ACTCTGTGAAGGGCCGATTCACCATCTCCAGGGACAACGCCAAGAA |
| | | | CTCACTGTATCTGCAAATTAACAGCCTGAGAGCCGAGGACACGGCC |
| | | | GTGTATTACTGTGCGAGAGATTTTTACGATATTTTGACTGATAGTC |
| | | | CGTACTTCTACTACGGTGTGGACGTCTGGGGCCAAGGGACCACGGT |
| | | | CACCGTCTCCTCA |
| 496 | STIM009 | Amino acid sequence | QVQLVESGGGLVKPGGSLRLSCAASGFTFSDYYMSWIRQAPGKGLE |
| | -full | of STIM009 heavy | WVSYISSSGSTIYYADSVKGRFTISRDNAKNSLYLQINSLRAEDTA |
| | heavy | chain | VYYCARDFYDILTDSPYFYYGVDVWGQGTTVTVSSASTKGPSVFPL |
| | chain | | APSSKSTSGGTAALGCLVKDYFPEPVTVSWNSGALTSGVHTFPAVL |
| | sequence | | QSSGLYSLSSVVTVPSSSLGTQTYICNVNHKPSNTKVDKKVEPKSC |
| | | | DKTHTCPPCPAPELLGGPSVFLFPPKPKDTLMISRTPEVTCVVVDV |
| | | | SHEDPEVKFNWYVDGVEVHNAKTKPREEQYNSTYRVVSVLTVLHQD |
| | | | WLNGKEYKCKVSNKALPAPIEKTISKAKGQPREPQVYTLPPSRDEL |
| | | | TKNQVSLTCLVKGFYPSDIAVEWESNGQPENNYKTTPPVLDSDGSF |
| | | | FLYSKLTVDKSRWQQGNVFSCSVMHEALHNHYTQKSLSLSPGK |
| 497 | STIM009 | Nucleic acid | CAGGTGCAGCTGGTGGAGTCTGGGGGAGGCTTGGTCAAGCCTGGAG |
| | -full | sequence of STIM009 | GGTCCCTGAGACTCTCCTGTGCAGCCTCTGGATTCACCTTCAGTGA |
| | heavy | heavy chain | CTACTACATGAGCTGGATCCGCCAGGCTCCAGGGAAGGGGCTGGAG |
| | chain | | TGGGTTTCATACATTAGTAGTAGTGGTAGTACCATATACTACGCAG |
| | sequence | | ACTCTGTGAAGGGCCGATTCACCATCTCCAGGGACAACGCCAAGAA |
| | | | CTCACTGTATCTGCAAATTAACAGCCTGAGAGCCGAGGACACGGCC |
| | | | GTGTATTACTGTGCGAGAGATTTTTACGATATTTTGACTGATAGTC |
| | | | CGTACTTCTACTACGGTGTGGACGTCTGGGGCCAAGGGACCACGGT |
| | | | CACCGTCTCCTCAGCCAGCACCAAGGGCCCCTCTGTGTTCCCTCTG |
| | | | GCCCCTTCCAGCAAGTCCACCTCTGGCGGAACAGCCGCTCTGGGCT |
| | | | GCCTCGTGAAGGACTACTTCCCCGAGCCTGTGACCGTGTCCTGGAA |
| | | | CTCTGGCGCTCTGACCAGCGGAGTGCACACCTTCCCTGCTGTGCTG |
| | | | CAGTCCTCCGGCCTGTACTCCCTGTCCTCCGTCGTGACCGTGCCTT |
| | | | CCAGCTCTCTGGGCACCCAGACCTACATCTGCAACGTGAACCACAA |
| | | | GCCCTCCAACACCAAGGTGGACAAGAAGGTGGAACCCAAGTCCTGC |
| | | | GACAAGACCCACACCTGTCCCCCTTGTCCTGCCCCTGAACTGCTGG |
| | | | GCGGACCTTCCGTGTTCCTGTTCCCCCCAAAGCCCAAGGACACCCT |
| | | | GATGATCTCCCGGACCCCCGAAGTGACCTGCGTGGTGGTGGATGTG |
| | | | TCCCACGAGGACCCTGAAGTGAAGTTCAATTGGTACGTGGACGGCG |
| | | | TGGAAGTGCACAACGCCAAGACCAAGCCTAGAGAGGAACAGTACAA |
| | | | CTCCACCTACCGGGTGGTGTCCGTGCTGACCGTGCTGCACCAGGAT |
| | | | TGGCTGAACGGCAAAGAGTACAAGTGCAAGGTGTCCAACAAGGCCC |
| | | | TGCCTGCCCCCATCGAAAAGACCATCTCCAAGGCCAAGGGCCAGCC |
| | | | CCGGGAACCCCAGGTGTACACACTGCCCCCTAGCAGGGACGAGCTG |
| | | | ACCAAGAACCAGGTGTCCCTGACCTGTCTCGTGAAAGGCTTCTACC |
| | | | CCTCCGATATCGCCGTGGAATGGGAGTCCAACGGCCAGCCTGAGAA |
| | | | CAACTACAAGACCACCCCCCCTGTGCTGGACTCCGACGGCTCATTC |
| | | | TTCCTGTACAGCAAGCTGACAGTGGACAAGTCCCGGTGGCAGCAGG |
| | | | GCAACGTGTTCTCCTGCTCCGTGATGCACGAGGCCCTGCACAACCA |
| | | | CTACACCCAGAAGTCCCTGTCCCTGAGCCCCGGCAAGTGATGA |
| 498 | STIM009- | Amino acid sequence | QSLLHSNGYNY |
| | CDRL1 | of CDRL1 of STIM009 | |
| | | using IMGT | |
| 499 | STIM009- | Amino acid sequence | LGS |
| | CDRL2 | of CDRL2 of STIM009 | |
| | | using IMGT | |
| 500 | STIM009- | Amino acid sequence | MQALQTPRT |
| | CDRL3 | of CDRL3 of STIM009 | |
| | | using IMGT | |
| 501 | STIM009 | Amino acid sequence | DIVMTQSPLSLPVTPGEPASISCRSSQSLLHSNGYNYLDWYLQKPG |
| | -Light | of V_{L} of STIM009 | QSPQLLIYLGSNRASGVPDRESGSGSGTDFTLKISRVEAEDVGVYY |
| | chain | | CMQALQTPRTFGQGTKVEIK |
| | variable | | |
| | region | | |
| 502 | STIM009 | Nucleic acid | GATATTGTGATGACTCAGTCTCCACTCTCCCTGCCCGTCACCCCTG |
| | -Light | sequence of V_{L} of | GAGAGCCGGCCTCCATCTCCTGCAGGTCTAGTCAGAGCCTCCTGCA |
| | chain | STIM009 | TAGTAATGGATACAACTATTTGGATTGGTACCTGCAGAAGCCAGGG |
| | variable | | CAGTCTCCACAGCTCCTGATCTATTTGGGTTCTAATCGGGCCTCCG |
| | region | | GGGTCCCTGACAGGTTCAGTGGCAGTGGATCAGGCACAGATTTTAC |
| | | | ACTGAAAATCAGCAGAGTGGAGGCTGAGGATGTTGGGGTTTATTAC |
| | | | TGCATGCAAGCTCTACAAACTCCTCGGACGTTCGGCCAAGGGACCA |
| | | | AGGTGGAAATCAAA |
| 503 | STIM009 | Amino acid sequence | DIVMTQSPLSLPVTPGEPASISCRSSQSLLHSNGYNYLDWYLQKPG |
| | -full | of STIM009 light | QSPQLLIYLGSNRASGVPDRFSGSGSGTDFTLKISRVEAEDVGVYY |
| | light | chain | CMQALQTPRTFGQGTKVEIKRTVAAPSVFIFPPSDEQLKSGTASVV |
| | chain | | CLLNNFYPREAKVQWKVDNALQSGNSQESVTEQDSKDSTYSLSSTL |
| | sequence | | TLSKADYEKHKVYACEVTHQGLSSPVTKSENRGEC |
| 504 | STIM009 | Nucleic acid | GATATTGTGATGACTCAGTCTCCACTCTCCCTGCCCGTCACCCCTG |
| | -full | sequence of STIM009 | GAGAGCCGGCCTCCATCTCCTGCAGGTCTAGTCAGAGCCTCCTGCA |
| | light | light chain | TAGTAATGGATACAACTATTTGGATTGGTACCTGCAGAAGCCAGGG |
| | chain | | CAGTCTCCACAGCTCCTGATCTATTTGGGTTCTAATCGGGCCTCCG |
| | sequence | | GGGTCCCTGACAGGTTCAGTGGCAGTGGATCAGGCACAGATTTTAC |
| | | | ACTGAAAATCAGCAGAGTGGAGGCTGAGGATGTTGGGGTTTATTAC |
| | | | TGCATGCAAGCTCTACAAACTCCTCGGACGTTCGGCCAAGGGACCA |
| | | | AGGTGGAAATCAAAcgtacggtggccgctccctccgtgttcatctt |
| | | | cccaccttccgacgagcagctgaagtccggcaccgcttctgtcgtg |
| | | | tgcctgctgaacaacttctacccccgcgaggccaaggtgcagtgga |
| | | | aggtggacaacgccctgcagtccggcaactcccaggaatccgtgac |
| | | | cgagcaggactccaaggacagcacctactccctgtcctccaccctg |
| | | | accctgtccaaggccgactacgagaagcacaaggtgtacgcctgcg |
| | | | aagtgacccaccagggcctgtctagccccgtgaccaagtotttcaa |
| | | | ccggggcgagtgt |
| 505 | Human | Amino acid sequence | FTVTVPKDLYVVEYGSNMTIECKFPVEKQLDLAALIVYWEMEDKNI |
| | PD-L1 | of KYPROT286 with | IQFVHGEEDLKVQHSSYRQRARLLKDQLSLGNAALQITDVKLQDAG |
| | Flag His | FLAG tag in bold | VYRCMISYGGADYKRITVKVNAPYNKINQRILVVDPVTSEHELTCQ |
| | (KYPROT2 | and underlined and | AEGYPKAEVIWTSSDHQVLSGKTTTTNSKREEKLENVTSTLRINTT |
| | 86) | histidine tag in | TNEIFYCTFRRLDPEENHTAELVIPELPLAHPPNERTIEGRDYKDD |
| | | bold | DDKHHHHHH |
| 506 | Mature | Mature amino acid | EINGSANYEMFIFHNGGVQILCKYPDIVQQFKMQLLKGGQILCDLT |
| | human | sequence of human | KTKGSGNTVSIKSLKFCHSQLSNNSVSFFLYNLDHSHANYYFCNLS |
| | ICOS | ICOS | IFDPPPFKVTLTGGYLHIYESQLCCQLKFWLPIGCAAFVVVCILGC |
| | | | ILICWLTKKKYSSSVHDPNGEYMFMRAVNTAKKSRLTDVTL |
| 507 | Human | Amino acid sequence | EINGSANYEMFIFHNGGVQILCKYPDIVQQFKMQLLKGGQILCDLT |
| | ICOS | of human ICOS | KTKGSGNTVSIKSLKFCHSQLSNNSVSFFLYNLDHSHANYYFCNLS |
| | extra- | extracellular | IFDPPPFKVTLTGGYLHIYESQLCCQLKF |
| | cellular | domain | |
| | domain | | |
| 508 | Human | Amino acid sequence | MKSGLWYFFLFCLRIKVLTGEINGSANYEMFIFHNGGVQILCKYPD |
| | ICOS | of human ICOS | IVQQFKMQLLKGGQILCDLTKTKGSGNTVSIKSLKFCHSQLSNNSV |
| | with | (signal peptide is | SFFLYNLDHSHANYYFCNLSIFDPPPFKVTLTGGYLHIYESQLCCQ |
| | signal | underlined) | LKFWLPIGCAAFVVVCILGCILICWLTKKKYSSSVHDPNGEYMEMR |
| | peptide | | AVNTAKKSRLTDVTL |
| 509 | Isoform | Amino acid sequence | The sequence of this isoform differs from the |
| | of human | of a human ICOS | canonical sequence in its cytoplasmic domain |
| | ICOS | isoform | as follows: 168-199: |
| | (Q9Y6W8- | | KYSSSVHDPNGEYMEMRAVNTAKKSRLTDVTLM |
| | 2) | | |
| 510 | Mature | Mature amino acid | EINGSADHRMFSFHNGGVQISCKYPETVQQLKMRLFREREVLCELT |
| | mouse | sequence of mouse | KTKGSGNAVSIKNPMLCLYHLSNNSVSFFLNNPDSSQGSYYFCSLS |
| | ICOS | ICOS | IFDPPPFQERNLSGGYLHIYESQLCCQLKIVVQVTE |
| 511 | Mouse | Amino acid sequence | EINGSADHRMFSFHNGGVQISCKYPETVQQLKMRLFREREVLCELT |
| | ICOS | of the | KTKGSGNAVSIKNPMLCLYHLSNNSVSFFLNNPDSSQGSYYFCSLS |
| | extra- | extracellular | IFDPPPFQERNLSGGYLHIYESQLCCQLK |
| | cellular | domain of mouse | |
| | domain | ICOS | |
| 512 | Mouse | Amino acid sequence | MGWSCIILFLVATATGVHSEINGSADHRMFSFHNGGVQISCKYPET |
| | ICOS | of mouse ICOS | VQQLKMRLFREREVLCELTKTKGSGNAVSIKNPMLCLYHLSNNSVS |
| | with | (signal peptide is | FFLNNPDSSQGSYYFCSLSIFDPPPFQERNLSGGYLHIYESQLCCQ |
| | signal | underlined) | LKIVVQVTE |
| | peptide | | |
| 513 | Cynomolgus | Amino acid sequence | MKSGLWYFFL FCLHMKVLTG EINGSANYEM FIFHNGGVQI |
| | ICOS | of cynomolgus ICOS | LCKYPDIVQQ |
| | with | | FKMQLLKGGQILCDLTKTKGSGNKVSIKSLKFCHSQLSNNSVSFFL |
| | signal | (signal peptide is | YNLDRSHANYYFCNLSIFDPPPFKVTLTGGYLHIYESQLCCQLKEW |
| | peptide | underlined) | LPIGCATEVVVCIFGCILICWLTKKKYSSTVHDPNGEYMEMRAVNT |
| | | | AKKSRLTGTTP |
| 514 | Cynomolgus | Amino acid sequence | EINGSANYEMFIFHNGGVQILCKYPDIVQQFKMQLLKGGQILCDLT |
| | ICOS | of cynomolgus ICOS | KTKGSGNKVSIKSLKFCHSQLSNNSVSFFLYNLDRSHANYYFCNLS |
| | extra- | extracellular | IFDPPPFKVTLTGGYLHIYESQLCCQLK |
| | cellular | domain | |
| | domain | | |
| 515 | Human | Amino acid sequence | DTQEKEVRAMVGSDVELSCACPEGSRFDLNDVYVYWQTSESKTVVT |
| | ICOS | of human ICOS | YHIPQNSSLENVDSRYRNRALMSPAGMLRGDFSLRLENVTPQDEQK |
| | ligand | ligand comprising | FHCLVLSQSLGFQEVLSVEVTLHVAANFSVPVVSAPHSPSQDELTE |
| | | extracellular | TCTSINGYPRPNVYWINKTDNSLLDQALQNDTVFLNMRGLYDVVSV |
| | | domain | LRIARTPSVNIGCCIENVLLQQNLTVGSQTGNDIGERDKITENPVS |
| | | | TGEKNAATWS |
| 516 | Human | Amino acid sequence | MRLGSPGLLFLLFSSLRADTQEKEVRAMVGSDVELSCACPEGSRED |
| | ICOS | of human ICOS | LNDVYVYWQTSESKTVVTYHIPQNSSLENVDSRYRNRALMSPAGML |
| | ligand | ligand including | RGDFSLRLFNVTPQDEQKFHCLVLSQSLGFQEVLSVEVTLHVAANF |
| | | signal peptide | SVPVVSAPHSPSQDELTFTCTSINGYPRPNVYWINKTDNSLLDQAL |
| | | | QNDTVFLNMRGLYDVVSVLRIARTPSVNIGCCIENVLLQQNLTVGS |
| | | | QTGNDIGERDKITENPVSTGEKNAATWSILAVLCLLVVVAVAIGWV |
| | | | CRDRCLQHSYAGAWAVSPETELTGHV |

SEQ ID NO: 610 ICOSL-Fc
DTQEKEVRAMVGSDVELSCACPEGSRFDLNDVYVYWQTSESKTVVTYHIPQNSSLENVDSRYRNRALMSPAGMLRGDFSLRLENVT
PQDEQKFHCLVLSQSLGFQEVLSVEVTLHVAANFSVPVVSAPHSPSQDELTFTCTSINGYPRPNVYWINKTDNSLLDQALQNDTVE
LNMRGLYDVVSVLRIARTPSVNIGCCIENVLLQQNLTVGSQTGNDIGERDKITENPVSTGEKNAATWSDIEGRMDPKSCDKTHTCP
PCPAPELLGGPSVFLFPPKPKDTLMISRTPEVTCVVVDVSHEDPEVKENWYVDGVEVHNAKTKPREEQYNSTYRVVSVLTVLHQDW
LNGKEYKCKVSNKALPAPIEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
Linker is underlined and in bold. Sequence preceding linker is human ICOSL (B7-H2) .
Sequence following linker is human IgG1 Fc.

| 517 | C-terminal amino | Amino acids 21 to | | LQMILNGINNYKNPKLTAMLTFKFYMPKKATELKHLQCL |
|---|---|---|---|---|
| | acid sequence of | 133 of hIL-2 with | | EEELKPLEEVLNLAQSKNFHLRPRDLISNINVIVLELKG |
| | hIL-2 | R38W mutation | | SETTEMCEYADETATIVEFLNRWITFCQSIISTLT |
| | | (bold & | | |
| | | underlined) | | |
| 518 | C-terminal amino | Amino acids 21 to | | LQMILNGINNYKNPKLTQMLTFKFYMPKKATELKHLQCL |
| | acid sequence of | 133 of hIL-2 with | | EEELKPLEEVLNLAQSKNFHLRPRDLISNINVIVLELKG |
| | hIL-2 | R38Q mutation | | SETTEMCEYADETATIVEFLNRWITFCQSIISTLT |
| | | (bold & | | |
| | | underlined) | | |
| 519 | STIM002- | Nucleic acid | | GATATTGTGATGACTCAGTCTCCACTCTCCCTGCCCGTC |
| | Corrected Light | sequence of | | ACCCCTGGAGAGCCGGCCTCCATCTCCTGCAGGTCTAGT |
| | chain variable | corrected V_{L} of | | CAGAGCCTCCTGCATAGTGATGGATACAACTATTTGGAT |
| | region | STIM002 | | TGGTACCTGCAGAAGCCAGGGCAGTCTCCACAGCTCCTG |
| | | | | ATCTATTTGGGTTCTACTCGGGCCTCCGGGTTCCCTGAC |
| | | | | AGGTTCAGTGGCAGTGGATCAGGCACAGATTTTACACTG |
| | | | | AAAATCAGCAGAGTGGAGGCTGAGGATGTTGGGGTTTAT |
| | | | | TACTGCATGCAAGCTCTACAAACTCCGCTCAGTTTTGGC |
| | | | | CAGGGGACCAAGCTGGAGATCAAA |
| 520 | STIM002- | Nucleic acid | | GATATTGTGATGACTCAGTCTCCACTCTCCCTGCCCGTC |
| | Corrected full | sequence of | | ACCCCTGGAGAGCCGGCCTCCATCTCCTGCAGGTCTAGT |
| | light chain | corrected STIM002 | | CAGAGCCTCCTGCATAGTGATGGATACAACTATTTGGAT |
| | sequence | light chain | | TGGTACCTGCAGAAGCCAGGGCAGTCTCCACAGCTCCTG |
| | | | | ATCTATTTGGGTTCTACTCGGGCCTCCGGGTTCCCTGAC |
| | | | | AGGTTCAGTGGCAGTGGATCAGGCACAGATTTTACACTG |
| | | | | AAAATCAGCAGAGTGGAGGCTGAGGATGTTGGGGTTTAT |
| | | | | TACTGCATGCAAGCTCTACAAACTCCGCTCAGTTTTGGC |
| | | | | CAGGGGACCAAGCTGGAGATCAAAcgtacggtggccgct |
| | | | | ccctccgtgttcatcttcccaccttccgacgagcagctg |
| | | | | aagtccggcaccgcttctgtcgtgtgcctgctgaacaac |
| | | | | ttctacccccgcgaggccaaggtgcagtggaaggtggac |
| | | | | aacgccctgcagtccggcaactcccaggaatccgtgacc |
| | | | | gagcaggactccaaggacagcacctactccctgtcctcc |
| | | | | accctgaccctgtccaaggccgactacgagaagcacaag |
| | | | | gtgtacgcctgcgaagtgacccaccagggcctgtctagc |
| | | | | cccgtgaccaagtctttcaaccggggcgagtgt |
| 521 | STIM003- | Nucleic acid | | GAGGTGCAGCTGGTGGAGTCTGGGGGAGGTGTGGTACGG |
| | Corrected heavy | sequence of | | CCTGGGGGGTCCCTGAGACTCTCCTGTGTAGCCTCTGGA |
| | chain variable | corrected V_{H} of | | GTCACCTTTGATGATTATGGCATGAGCTGGGTCCGCCAA |
| | region | STIM003 | | GCTCCAGGGAAGGGGCTGGAGTGGGTCTCTGGTATTAAT |
| | | | | TGGAATGGTGGCGACACAGATTATTCAGACTCTGTGAAG |
| | | | | GGCCGATTCACCATCTCCAGAGACAACGCCAAGAACTCC |
| | | | | CTGTATCTACAAATGAATAGTCTGAGAGCCGAGGACACG |
| | | | | GCCTTGTATTACTGTGCGAGGGATTTCTATGGTTCGGGG |
| | | | | AGTTATTATCACGTTCCTTTTGACTACTGGGGCCAGGGA |
| | | | | ATCCTGGTCACCGTCTCCTCA |
| 522 | STIM003- | Nucleic acid | | GAGGTGCAGCTGGTGGAGTCTGGGGGAGGTGTGGTACGG |
| | Corrected full | sequence of | | CCTGGGGGGTCCCTGAGACTCTCCTGTGTAGCCTCTGGA |
| | heavy chain | corrected STIM003 | | GTCACCTTTGATGATTATGGCATGAGCTGGGTCCGCCAA |
| | sequence | heavy chain | | GCTCCAGGGAAGGGGCTGGAGTGGGTCTCTGGTATTAAT |
| | | | | TGGAATGGTGGCGACACAGATTATTCAGACTCTGTGAAG |
| | | | | GGCCGATTCACCATCTCCAGAGACAACGCCAAGAACTCC |
| | | | | CTGTATCTACAAATGAATAGTCTGAGAGCCGAGGACACG |
| | | | | GCCTTGTATTACTGTGCGAGGGATTTCTATGGTTCGGGG |
| | | | | AGTTATTATCACGTTCCTTTTGACTACTGGGGCCAGGGA |
| | | | | ATCCTGGTCACCGTCTCCTCAGCCAGCACCAAGGGCCCC |
| | | | | TCTGTGTTCCCTCTGGCCCCTTCCAGCAAGTCCACCTCT |
| | | | | GGCGGAACAGCCGCTCTGGGCTGCCTCGTGAAGGACTAC |
| | | | | TTCCCCGAGCCTGTGACCGTGTCCTGGAACTCTGGCGCT |
| | | | | CTGACCAGCGGAGTGCACACCTTCCCTGCTGTGCTGCAG |
| | | | | TCCTCCGGCCTGTACTCCCTGTCCTCCGTCGTGACCGTG |
| | | | | CCTTCCAGCTCTCTGGGCACCCAGACCTACATCTGCAAC |
| | | | | GTGAACCACAAGCCCTCCAACACCAAGGTGGACAAGAAG |
| | | | | GTGGAACCCAAGTCCTGCGACAAGACCCACACCTGTCCC |
| | | | | CCTTGTCCTGCCCCTGAACTGCTGGGCGGACCTTCCGTG |
| | | | | TTCCTGTTCCCCCCAAAGCCCAAGGACACCCTGATGATC |
| | | | | TCCCGGACCCCCGAAGTGACCTGCGTGGTGGTGGATGTG |
| | | | | TCCCACGAGGACCCTGAAGTGAAGTTCAATTGGTACGTG |
| | | | | GACGGCGTGGAAGTGCACAACGCCAAGACCAAGCCTAGA |
| | | | | GAGGAACAGTACAACTCCACCTACCGGGTGGTGTCCGTG |
| | | | | CTGACCGTGCTGCACCAGGATTGGCTGAACGGCAAAGAG |
| | | | | TACAAGTGCAAGGTGTCCAACAAGGCCCTGCCTGCCCCC |
| | | | | ATCGAAAAGACCATCTCCAAGGCCAAGGGCCAGCCCCGG |
| | | | | GAACCCCAGGTGTACACACTGCCCCCTAGCAGGGACGAG |
| | | | | CTGACCAAGAACCAGGTGTCCCTGACCTGTCTCGTGAAA |
| | | | | GGCTTCTACCCCTCCGATATCGCCGTGGAATGGGAGTCC |
| | | | | AACGGCCAGCCTGAGAACAACTACAAGACCACCCCCCCT |
| | | | | GTGCTGGACTCCGACGGCTCATTCTTCCTGTACAGCAAG |
| | | | | CTGACAGTGGACAAGTCCCGGTGGCAGCAGGGCAACGTG |
| | | | | TTCTCCTGCTCCGTGATGCACGAGGCCCTGCACAACCAC |
| | | | | TACACCCAGAAGTCCCTGTCCCTGAGCCCCGGCAAGTGA |
| | | | | TGA |
| 523 | Human IgG1 | IGHG | Human Heavy Chain | tcctccaagagcacctctgggggcacagcggccctgggc |
| | | 1*03 | Constant Region | tgcctggtcaaggactacttccccgaaccggtgacggtg |
| | | | (IGHG1*03) | tcgtggaactcaggcgccctgaccagcggcgtgcacacc |
| | | | Nucleotide | ttcccggctgtcctacagtcctcaggactctactccctc |
| | | | Sequence | agcagcgtggtgaccgtgccctccagcagcttgggcacc |
| | | | | cagacctacatctgcaacgtgaatcacaagcccagcaac |
| | | | | accaaggtggacaagagagttgagcccaaatcttgtgac |
| | | | | aaaactcacacatgcccaccgtgcccagcacctgaactc |
| | | | | ctggggggaccgtcagtcttcctcttccccccaaaaccc |
| | | | | aaggacaccctcatgatctcccggacccctgaggtcaca |
| | | | | tgcgtggtggtggacgtgagccacgaagaccctgaggtc |
| | | | | aagttcaactggtacgtggacggcgtggaggtgcataat |
| | | | | gccaagacaaagccgcgggaggagcagtacaacagcacg |
| | | | | taccgtgtggtcagcgtcctcaccgtcctgcaccaggac |
| | | | | tggctgaatggcaaggagtacaagtgcaaggtctccaac |
| | | | | aaagccctcccagcccccatcgagaaaaccatctccaaa |
| | | | | gccaaagggcagccccgagaaccacaggtgtacaccctg |
| | | | | cccccatcccgggaggagatgaccaagaaccaggtcagc |
| | | | | ctgacctgcctggtcaaaggcttctatcccagcgacatc |
| | | | | gccgtggagtgggagagcaatgggcagccggagaacaac |
| | | | | tacaagaccacgcctcccgtgctggactccgacggctcc |
| | | | | ttcttcctctatagcaagctcaccgtggacaagagcagg |
| | | | | tggcagcaggggaacgtcttctcatgctccgtgatgcat |
| | | | | gaggctctgcacaaccactacacgcagaagagcctctcc |
| | | | | ctgtccccgggtaaa |
| 524 | constant | | Human Heavy Chain | A S T K G P S V F P L A P S S K S T S G |
| | region | | Constant Region | G T A A L G C L V K D Y F P E P V T V S |
| | | | (IGHG1*03) Protein | W N S G A L T S G V H T F P A V L Q S S |
| | | | Sequence | G L Y S L S S V V T V P S S S L G T Q T |
| | | | | Y I C N V N H K P S N T K V D K R V E P |
| | | | | K S C D K T H T C P P C P A P E L L G G |
| | | | | P S V F L F P P K P K D Ţ L M I S R T P |
| | | | | E V T C V V V D V S H E D P E V K F N W |
| | | | | Y V D G V E V H N A K T K P R E E Q Y N |
| | | | | S T Y R V V S V L T V L H Q D W L N G K |
| | | | | E Y K C K V S N K A L P A P I E K T I S |
| | | | | K A K G Q P R E P Q V Y T L P P S R E E |
| | | | | M T K N Q V S L T C L V K G F Y P S D I |
| | | | | A V E W E S N G Q P E N N Y K T T P P V |
| | | | | L D S D G S F F L Y S K L T V D K S R W |
| | | | | Q Q G N V F S C S V M H E A L H N H Y T |
| | | | | Q K S L S L S P G K |
| 525 | Human IgG1 | IGHG | Human Heavy Chain | tcctccaagagcacctctgggggcacagcggccctgggc |
| | constant | 1*04 | Constant Region | tgcctggtcaaggactacttccccgaaccggtgacggtg |
| | region | | (IGHG1*04) | tcgtggaactcaggcgccctgaccagcggcgtgcacacc |
| | | | Nucleotide | ttcccggctgtcctacagtcctcaggactctactccctc |
| | | | Sequence | agcagcgtggtgaccgtgccctccagcagcttgggcacc |
| | | | | cagacctacatctgcaacgtgaatcacaagcccagcaac |
| | | | | accaaggtggacaagaaagttgagcccaaatcttgtgac |
| | | | | aaaactcacacatgcccaccgtgcccagcacctgaactc |
| | | | | ctggggggaccgtcagtcttcctcttccccccaaaaccc |
| | | | | aaggacaccctcatgatctcccggacccctgaggtcaca |
| | | | | tgcgtggtggtggacgtgagccacgaagaccctgaggtc |
| | | | | aagttcaactggtacgtggacggcgtggaggtgcataat |
| | | | | gccaagacaaagccgcgggaggagcagtacaacagcacg |
| | | | | taccgtgtggtcagcgtcctcaccgtcctgcaccaggac |
| | | | | tggctgaatggcaaggagtacaagtgcaaggtctccaac |
| | | | | aaagccctcccagcccccatcgagaaaaccatctccaaa |
| | | | | gccaaagggcagccccgagaaccacaggtgtacaccctg |
| | | | | cccccatcccgggatgagctgaccaagaaccaggtcagc |
| | | | | ctgacctgcctggtcaaaggcttctatcccagcgacatc |
| | | | | gccgtggagtgggagagcaatgggcagccggagaacaac |
| | | | | tacaagaccacgcctcccgtgctggactccgacggctcc |
| | | | | ttcttcctctacagcaagctcaccgtggacaagagcagg |
| | | | | tggcagcaggggaacatcttctcatgctccgtgatgcat |
| | | | | gaggctctgcacaaccactacacgcagaagagcctctcc |
| | | | | ctgtctccgggtaaa |
| 526 | | | Human Heavy Chain | ASTKGPSVFPLAPSSKSTSGGTAALGCLVKDYFPEPVTV |
| | | | Constant Region | SWNSGALTSGVHTFPAVLQSSGLYSLSSVVTVPSSSLGT |
| | | | (IGHG1*04) Protein | QTYICNVNHKPSNTKVDKKVEPKSCDKTHTCPPCPAPEL |
| | | | Sequence | LGGPSVFLFPPKPKDTLMISRTPEVTCVVVDVSHEDPEV |
| | | | | KFNWYVDGVEVHNAKTKPREEQYNSTYRVVSVLTVLHQD |
| | | | | WLNGKEYKCKVSNKALPAPIEKTISKAKGQPREPQVYTL |
| | | | | PPSRDELTKNQVSLTCLVKGFYPSDIAVEWESNGQPENN |
| | | | | YKTTPPVLDSDGSFFLYSKLTVDKSRWQQGNIFSCSVMH |
| | | | | EALHNHYTQKSLSLSPGK |
| 527 | Human IgG2 | IGHG | Human Heavy Chain | gcctccaccaagggcccatcggtcttccccctggcgccc |
| | constant | 2*01 | Constant Region | tgctccaggagcacctccgagagcacagccgccctgggc |
| | region | & | (IGHG2*01) | tgcctggtcaaggactacttccccgaaccggtgacggtg |
| | | IGHG | Nucleotide | tcgtggaactcaggcgctctgaccagcggcgtgcacacc |
| | | 2*03 | Sequence | ttcccagctgtcctacagtcctcaggactctactccctc |
| | | & | | agcagcgtggtgaccgtgccctccagcaacttcggcacc |
| | | IGHG | | cagacctacacctgcaacgtagatcacaagcccagcaac |
| | | 2*05 | | accaaggtggacaagacagttgagcgcaaatgttgtgtc |
| | | | | gagtgcccaccgtgcccagcaccacctgtggcaggaccg |
| | | | | tcagtcttcctcttccccccaaaacccaaggacaccctc |
| | | | | atgatctcccggacccctgaggtcacgtgcgtggtggtg |
| | | | | gacgtgagccacgaagaccccgaggtccagttcaactgg |
| | | | | tacgtggacggcgtggaggtgcataatgccaagacaaag |
| | | | | ccacgggaggagcagttcaacagcacgttccgtgtggtc |
| | | | | agcgtcctcaccgttgtgcaccaggactggctgaacggc |
| | | | | aaggagtacaagtgcaaggtctccaacaaaggcctocca |
| | | | | gcccccatcgagaaaaccatctccaaaaccaaagggcag |
| | | | | ccccgagaaccacaggtgtacaccctgcccccatcccgg |
| | | | | gaggagatgaccaagaaccaggtcagcctgacctgcctg |
| | | | | gtcaaaggcttctaccccagcgacatcgccgtggagtgg |
| | | | | gagagcaatgggcagccggagaacaactacaagaccaca |
| | | | | cctcccatgctggactccgacggctccttcttcctctac |
| | | | | agcaagctcaccgtggacaagagcaggtggcagcagggg |
| | | | | aacgtcttctcatgctccgtgatgcatgaggctctgcac |
| | | | | aaccactacacgcagaagagcctctccctgtctccgggt |
| | | | | aaa |
| 528 | | | Human Heavy Chain | SWNSGALTSGVHTFPAVLQSSGLYSLSSVVTVPSSNFGT |
| | | | Constant Region | QTYTCNVDHKPSNTKVDKTVERKCCVECPPCPAPPVAGP |
| | | | (IGHG2*01) | SVFLFPPKPKDTLMISRTPEVTCVVVDVSHEDPEVQENW |
| | | | Protein | YVDGVEVHNAKTKPREEQFNSTFRVVSVLTVVHQDWLNG |
| | | | Sequence | KEYKCKVSNKGLPAPIEKTISKTKGQPREPQVYTLPPSR |
| | | | | EEMTKNQVSLTCLVKGFYPSDIAVEWESNGQPENNYKTT |
| | | | | PPMLDSDGSFFLYSKLTVDKSRWQQGNVFSCSVMHEALH |
| | | | | NHYTQKSLSLSPGK |
| 529 | Human IgG2 | IGHG | Human Heavy Chain | GCCTCCACCAAGGGCCCATCGGTCTTCCCCCTGGCGCCC |
| | constant | 2*02 | Constant Region | TGCTCCAGGAGCACCTCCGAGAGCACAGCGGCCCTGGGC |
| | | | (IGHG2*02) | TGCCTGGTCAAGGACTACTTCCCCGAACCGGTGACGGTG |
| | | | Nucleotide | TCGTGGAACTCAGGCGCTCTGACCAGCGGCGTGCACACC |
| | | | Sequence | TTCCCGGCTGTCCTACAGTCCTCAGGACTCTACTCCCTC |
| | | | | AGCAGCGTGGTGACCGTGACCTCCAGCAACTTCGGCACC |
| | | | | CAGACCTACACCTGCAACGTAGATCACAAGCCCAGCAAC |
| | | | | ACCAAGGTGGACAAGACAGTTGAGCGCAAATGTTGTGTC |
| | | | | GAGTGCCCACCGTGCCCAGCACCACCTGTGGCAGGACCG |
| | | | | TCAGTCTTCCTCTTCCCCCCAAAACCCAAGGACACCCTC |
| | | | | ATGATCTCCCGGACCCCTGAGGTCACGTGCGTGGTGGTG |
| | | | | GACGTGAGCCACGAAGACCCCGAGGTCCAGTTCAACTGG |
| | | | | TACGTGGACGGCATGGAGGTGCATAATGCCAAGACAAAG |
| | | | | CCACGGGAGGAGCAGTTCAACAGCACGTTCCGTGTGGTC |
| | | | | AGCGTCCTCACCGTCGTGCACCAGGACTGGCTGAACGGC |
| | | | | AAGGAGTACAAGTGCAAGGTCTCCAACAAAGGCCTCCCA |
| | | | | GCCCCCATCGAGAAAACCATCTCCAAAACCAAAGGGCAG |
| | | | | CCCCGAGAACCACAGGTGTACACCCTGCCCCCATCCCGG |
| | | | | GAGGAGATGACCAAGAACCAGGTCAGCCTGACCTGCCTG |
| | | | | GTCAAAGGCTTCTACCCCAGCGACATCGCCGTGGAGTGG |
| | | | | GAGAGCAATGGGCAGCCGGAGAACAACTACAAGACCACA |
| | | | | CCTCCCATGCTGGACTCCGACGGCTCCTTCTTCCTCTAC |
| | | | | AGCAAGCTCACCGTGGACAAGAGCAGGTGGCAGCAGGGG |
| | | | | AACGTCTTCTCATGCTCCGTGATGCATGAGGCTCTGCAC |
| | | | | AACCACTACACACAGAAGAGCCTCTCCCTGTCTCCGGGT |
| | | | | AAA |
| 530 | region | | Human Heavy Chain | ASTKGPSVFPLAPCSRSTSESTAALGCLVKDYFPEPVTV |
| | | | Constant Region | SWNSGALTSGVHTFPAVLQSSGLYSLSSVVTVTSSNFGT |
| | | | (IGHG2*02) Protein | QTYTCNVDHKPSNTKVDKTVERKCCVECPPCPAPPVAGP |
| | | | Sequence | SVFLFPPKPKDTLMISRTPEVTCVVVDVSHEDPEVQENW |
| | | | | YVDGMEVHNAKTKPREEQFNSTFRVVSVLTVVHQDWING |
| | | | | KEYKCKVSNKGLPAPIEKTISKTKGQPREPQVYTLPPSR |
| | | | | EEMTKNQVSLTCLVKGFYPSDIAVEWESNGQPENNYKTT |
| | | | | PPMLDSDGSFFLYSKLTVDKSRWQQGNVFSCSVMHEALH |
| | | | | NHYTQKSLSLSPGK |
| 531 | Human IgG2 | IGHG | Human Heavy Chain | tgctccaggagcacctccgagagcacagcggccctgggc |
| | constant | 2*04 | Constant Region | tgcctggtcaaggactacttccccgaaccggtgacggtg |
| | | | (IGHG2*04) | tcgtggaactcaggcgctctgaccagcggcgtgcacacc |
| | | | Nucleotide | ttcccagctgtcctacagtcctcaggactctactccctc |
| | | | Sequence | agcagcgtggtgaccgtgccctccagcagcttgggcacc |
| | | | | cagacctacacctgcaacgtagatcacaagcccagcaac |
| | | | | accaaggtggacaagacagttgagcgcaaatgttgtgtc |
| | | | | gagtgcccaccgtgcccagcaccacctgtggcaggaccg |
| | | | | tcagtcttcctcttccccccaaaacccaaggacaccctc |
| | | | | atgatctcccggacccctgaggtcacgtgcgtggtggtg |
| | | | | gacgtgagccacgaagaccccgaggtccagttcaactgg |
| | | | | tacgtggacggcgtggaggtgcataatgccaagacaaag |
| | | | | ccacgggaggagcagttcaacagcacgttccgtgtggtc |
| | | | | agcgtcctcaccgttgtgcaccaggactggctgaacggc |
| | | | | aaggagtacaagtgcaaggtctccaacaaaggcctocca |
| | | | | gcccccatcgagaaaaccatctccaaaaccaaagggcag |
| | | | | ccccgagaaccacaggtgtacaccctgcccccatcccgg |
| | | | | gaggagatgaccaagaaccaggtcagcctgacctgcctg |
| | | | | gtcaaaggcttctaccccagcgacatcgccgtggagtgg |
| | | | | gagagcaatgggcagccggagaacaactacaagaccaca |
| | | | | cctcccatgctggactccgacggctccttcttcctctac |
| | | | | agcaagctcaccgtggacaagagcaggtggcagcagggg |
| | | | | aacgtcttctcatgctccgtgatgcatgaggctctgcac |
| | | | | aaccactacacgcagaagagcctctccctgtctccgggt |
| | | | | aaa |
| 532 | region | | Human Heavy Chain | ASTKGPSVFPLAPCSRSTSESTAALGCLVKDYFPEPVTV |
| | | | Constant Region | SWNSGALTSGVHTFPAVLQSSGLYSLSSVVTVPSSSLGT |
| | | | (IGHG2*04) Protein | QTYTCNVDHKPSNTKVDKTVERKCCVECPPCPAPPVAGP |
| | | | Sequence | SVFLFPPKPKDTLMISRTPEVTCVVVDVSHEDPEVQENW |
| | | | | YVDGVEVHNAKTKPREEQFNSTFRVVSVLTVVHQDWLNG |
| | | | | KEYKCKVSNKGLPAPIEKTISKTKGQPREPQVYTLPPSR |
| | | | | EEMTKNQVSLTCLVKGFYPSDIAVEWESNGQPENNYKTT |
| | | | | PPMLDSDGSFFLYSKLTVDKSRWQQGNVFSCSVMHEALH |
| | | | | NHYTQKSLSLSPGK |
| 533 | Human IgG2 | IGHG | Human Heavy Chain | GCCTCCACCAAGGGCCCATCGGTCTTCCCCCTGGCGCCC |
| | constant | 2*06 | Constant Region | TGCTCCAGGAGCACCTCCGAGAGCACAGCGGCCCTGGGC |
| | region | | (IGHG2*06) | TGCCTGGTCAAGGACTACTTCCCCGAACCGGTGACGGTG |
| | | | Nucleotide | TCGTGGAACTCAGGCGCTCTGACCAGCGGCGTGCACACC |
| | | | Sequence | TTCCCGGCTGTCCTACAGTCCTCAGGACTCTACTCCCTC |
| | | | | AGCAGCGTGGTGACCGTGCCCTCCAGCAACTTCGGCACC |
| | | | | CAGACCTACACCTGCAACGTAGATCACAAGCCCAGCAAC |
| | | | | ACCAAGGTGGACAAGACAGTTGAGCGCAAATGTTGTGTC |
| | | | | GAGTGCCCACCGTGCCCAGCACCACCTGTGGCAGGACCG |
| | | | | TCAGTCTTCCTCTTCCCCCCAAAACCCAAGGACACCCTC |
| | | | | ATGATCTCCCGGACCCCTGAGGTCACGTGCGTGGTGGTG |
| | | | | GACGTGAGCCACGAAGACCCCGAGGTCCAGTTCAACTGG |
| | | | | TACGTGGACGGCGTGGAGGTGCATAATGCCAAGACAAAG |
| | | | | CCACGGGAGGAGCAGTTCAACAGCACGTTCCGTGTGGTC |
| | | | | AGCGTCCTCACCGTCGTGCACCAGGACTGGCTGAACGGC |
| | | | | AAGGAGTACAAGTGCAAGGTCTCCAACAAAGGCCTCCCA |
| | | | | GCCCCCATCGAGAAAACCATCTCCAAAACCAAAGGGCAG |
| | | | | CCCCGAGAACCACAGGTGTACACCCTGCCCCCATCCCGG |
| | | | | GAGGAGATGACCAAGAACCAGGTCAGCCTGACCTGCCTG |
| | | | | GTCAAAGGCTTCTACCCCAGCGACATCTCCGTGGAGTGG |
| | | | | GAGAGCAATGGGCAGCCGGAGAACAACTACAAGACCACA |
| | | | | CCTCCCATGCTGGACTCCGACGGCTCCTTCTTCCTCTAC |
| | | | | AGCAAGCTCACCGTGGACAAGAGCAGGTGGCAGCAGGGG |
| | | | | AACGTCTTCTCATGCTCCGTGATGCATGAGGCTCTGCAC |
| | | | | AACCACTACACACAGAAGAGCCTCTCCCTGTCTCCGGGT |
| | | | | AAA |
| 534 | | | Human Heavy Chain | SWNSGALTSGVHTFPAVLQSSGLYSLSSVVTVPSSNFGT |
| | | | Constant Region | QTYTCNVDHKPSNTKVDKTVERKCCVECPPCPAPPVAGP |
| | | | (IGHG2*06) Protein | SVFLFPPKPKDTLMISRTPEVTCVVVDVSHEDPEVQFNW |
| | | | Sequence | YVDGVEVHNAKTKPREEQFNSTFRVVSVLTVVHQDWLNG |
| | | | | KEYKCKVSNKGLPAPIEKTISKTKGQPREPQVYTLPPSR |
| | | | | EEMTKNQVSLTCLVKGFYPSDISVEWESNGQPENNYKTT |
| | | | | PPMLDSDGSFFLYSKLTVDKSRWQQGNVFSCSVMHEALH |
| | | | | NHYTQKSLSLSPGK |
| 535 | Human CA | IGLC | Cλ Light Chain | GGTCAGCCCAAGGCTGCCCCCTCGGTCACTCTGTTCCCA |
| | constant | 7*03 | Constant Region | CCCTCCTCTGAGGAGCTTCAAGCCAACAAGGCCACACTG |
| | region | | (IGLC7*03) | GTGTGTCTCGTAAGTGACTTCAACCCGGGAGCCGTGACA |
| | | | Nucleotide | GTGGCCTGGAAGGCAGATGGCAGCCCCGTCAAGGTGGGA |
| | | | Sequence | GTGGAGACCACCAAACCCTCCAAACAAAGCAACAACAAG |
| | | | | TATGCGGCCAGCAGCTACCTGAGCCTGACGCCCGAGCAG |
| | | | | TGGAAGTCCCACAGAAGCTACAGCTGCCGGGTCACGCAT |
| | | | | GAAGGGAGCACCGTGGAGAAGACAGTGGCCCCTGCAGAA |
| | | | | TGCTCT |
| 536 | | | Cλ Light Chain | GQPKAAPSVTLFPPSSEELQANKATLVCLVSDENPGAVT |
| | | | Constant Region | VAWKADGSPVKVGVETTKPSKQSNNKYAASSYLSLTPEQ |
| | | | (IGLC7*03) Amino | WKSHRSYSCRVTHEGSTVEKTVAPAECS |
| | | | Acid Sequence | |
| 537 | Human WT | IGHG | WT human IgG1 | gcctccaccaagggcccatcggtcttccccctggcaccc |
| | IgG1 | 1*01 | nucleotide | tcctccaagagcacctctgggggcacagcggccctgggc |
| | constant | & | sequence #2 | tgcctggtcaaggactacttccccgaaccggtgacggtg |
| | region | IGHG | | tcgtggaactcaggcgccctgaccagcggcgtgcacacc |
| | | 1*05 | | ttcccggctgtcctacagtcctcaggactctactccctc |
| | | (IgG | | agcagcgtggtgaccgtgccctccagcagcttgggcacc |
| | | 1) | | cagacctacatctgcaacgtgaatcacaagcccagcaac |
| | | | | accaaggtggacaagaaagttgagcccaaatcttgtgac |
| | | | | aaaactcacacatgcccaccgtgcccagcacctgaactc |
| | | | | ctggggggaccgtcagtcttcctcttccccccaaaaccc |
| | | | | aaggacaccctcatgatctcccggacccctgaggtcaca |
| | | | | tgcgtggtggtggacgtgagccacgaagaccctgaggtc |
| | | | | aagttcaactggtacgtggacggcgtggaggtgcataat |
| | | | | gccaagacaaagccgcgggaggagcagtacaacagcacg |
| | | | | taccgggtggtcagcgtcctcaccgtcctgcaccaggac |
| | | | | tggctgaatggcaaggagtacaagtgcaaggtctccaac |
| | | | | aaagccctcccagcccccatcgagaaaaccatctccaaa |
| | | | | gccaaagggcagccccgagaaccacaggtgtacaccctg |
| | | | | cccccatcccgggatgagctgaccaagaaccaggtcagc |
| | | | | ctgacctgcctggtcaaaggcttctatcccagcgacatc |
| | | | | gccgtggagtgggagagcaatgggcagccggagaacaac |
| | | | | tacaagaccacgcctcccgtgctggactccgacggctcc |
| | | | | ttcttcctctacagcaagctcaccgtggacaagagcagg |
| | | | | tggcagcaggggaacgtcttctcatgctccgtgatgcat |
| | | | | gaggctctgcacaaccactacacgcagaagagcctctcc |
| | | | | ctgtctccgggtaaa |
| 538 | Human CA | IGLC | Cλ Light Chain | GQPKAAPSVTLFPPSSEELQANKATLVCLISDFYPGAVT |
| | constant | 2*01 | Constant Region | VAWKADSSPVKAGVETTTPSKQSNNKYAASSYLSLTPEQ |
| | region | | Amino Acid | WKSHRSYSCQVTHEGSTVEKTVAPTECS |
| | | | Sequence #2- | |
| | | | Encoded by | |
| | | | nucleotide | |
| | | | sequence | |
| | | | version A & B | |

TABLE S3
SEQ ID NOS: 539-562

Sequence

hIgG1 FIT-Ig bispecific 1a

| | | |
|---|---|---|
| Antibody A | anti-ICOS | |
| | STIM003 | |
| Antibody B | anti-PD-L1 | |
| | 84G09 | |
| FIT-Ig | SEQ ID NO: | DIQMTQSPASLSASLGETVTIQCRASEDIYSGLAWFQQKPGKSPQLLIYGASS |
| Construct #1 | 539 | LQDGVPSRFSGSGSGTQYSLKISSMQTEDEGVYFCQQGLKYPPTFGSGTKLEI |
| | | KRTVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNRG |
| | | ECEVQLVESGGGLTQPGKSLKLSCEASGFTFSSFTMHWVRQSPGKGLEWVAFI |
| | | RSGSGIVFYADAVRGRFTISRDNAKNLLFLQMNDLKSEDTAMYYCARRPLGHN |
| | | TFDSWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSN |
| | | TKVDKKVEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPSRDELTKNQVSLTC |
| | | LVKGFYPSDIAVEWESNGQPENNYKTTPPVLDSDGSFFLYSKLTVDKSRWQQG |
| | | NVFSCSVMHEALHNHYTQKSLSLSPGK |
| FIT-Ig | SEQ ID NO: | EVQLVESGGGLVQPGRSLKLSCAASGFTFSDFYMAWVRQAPKKGLEWVASISY |
| Construct #2 | 540 | EGSSTYYGDSVMGRFTISRDNAKSTLYLQMNSLRSEDTATYYCARQREANWED |
| | | WGQGVMVTVSSASTKGPSVFPLAPSSKSTSGGTAALGCLVKDYFPEPVTVSWN |
| | | SGALTSGVHTFPAVLQSSGLYSLSSVVTVPSSSLGTQTYICNVNHKPSNTKVD |
| | | KKV |
| FIT-Ig | SEQ ID NO: | DIVMTQSPSSLAVSPGEKVTMTCKSSQSLYYSGVKENLLAWYQQKPGQSPKLL |
| Construct #3 | 541 | IYYASIRFTGVPDRFTGSGSGTDYTLTITSVQAEDMGQYFCQQGINNPLTFGD |
| | | GTKLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGEC |

hIgG1 FIT-Ig bispecific 1b

| | | |
|---|---|---|
| Antibody A | anti-PD-L1 | |
| | 84G09 | |
| Antibody B | anti-ICOS | |
| | STIM003 | |
| FIT-Ig | SEQ ID NO: | DIVMTQSPSSLAVSPGEKVTMTCKSSQSLYYSGVKENLLAWYQQKPGQSPKLL |
| Construct #1 | 542 | IYYASIRFTGVPDRFTGSGSGTDYTLTITSVQAEDMGQYFCQQGINNPLTFGD |
| | | GTKLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGECEVQLVESGGGLVQPGRSLKLSCAASGFTFSDFYMAWVRQAPKKGL |
| | | EWVASISYEGSSTYYGDSVMGRFTISRDNAKSTLYLQMNSLRSEDTATYYCAR |
| | | QREANWEDWGQGVMVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVNH |
| | | KPSNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTVLH |
| | | QDWLNGKEYKCKVSNKALPAPIEKTISKAKGQPREPQVYTLPPSRDELTKNQV |
| | | SLTCLVKGFYPSDIAVEWESNGQPENNYKTTPPVLDSDGSFFLYSKLTVDKSR |
| | | WQQGNVFSCSVMHEALHNHYTQKSLSLSPGK |
| FIT-Ig | SEQ ID NO: | EVQLVESGGGLTQPGKSLKLSCEASGFTFSSFTMHWVRQSPGKGLEWVAFIRS |
| Construct #2 | 543 | GSGIVFYADAVRGRFTISRDNAKNLLFLQMNDLKSEDTAMYYCARRPLGHNTF |
| | | DSWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTK |
| | | VDKKV |
| FIT-Ig | SEQ ID NO: | DIQMTQSPASLSASLGETVTIQCRASEDIYSGLAWFQQKPGKSPQLLIYGASS |
| Construct #3 | 544 | LQDGVPSRFSGSGSGTQYSLKISSMQTEDEGVYFCQQGLKYPPTFGSGTKLEI |
| | | KRTVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNRG |
| | | EC |

hIgG1 FIT-Ig bispecific 2a

| | | |
|---|---|---|
| Antibody A | anti-ICOS | |
| | STIM001 | |
| Antibody B | anti-PD-L1 | |
| | 1D05 | |
| FIT-Ig | SEQ ID NO: | DIQMTQSPASLSASLGETVTIQCRASEDIYSGLAWFQQKPGKSPQLLIYGASS |
| Construct #1 | 545 | LQDGVPSRFSGSGSGTQYSLKISSMQTEDEGVYFCQQGLKYPPTFGSGTKLEI |
| | | KRTDAAPTVSIFPPSSEQLTSGGASVVCFLNNFYPKDINVKWKIDGSERQNGV |
| | | LNSWTDQDSKDSTYSMSSTLTLTKDEYERHNSYTCEATHKTSTSPIVKSFNRN |
| | | ECEVQLVESGGGLTQPGKSLKLSCEASGFTFSSFTMHWVRQSPGKGLEWVAFI |
| | | RSGSGIVFYADAVRGRFTISRDNAKNLLFLQMNDLKSEDTAMYYCARRPLGHN |
| | | TFDSWGQGTLVTVSSAKTTAPSVYPLAPVCGDTTGSSVTLGCLVKGYFPEPVT |
| | | LTWNSGSLSSGVHTFPAVLQSDLYTLSSSVTVTSSTWPSQSITCNVAHPASST |
| | | KVDKKIEPRGPTIKPCPPCKCPAPNLLGGPSVFIFPPKIKDVLMISLSPIVTC |
| | | VVVDVSEDDPDVQISWFVNNVEVHTAQTQTHREDYNSTLRVVSALPIQHQDWM |
| | | SGKEFKCKVNNKDLPAPIERTISKPKGSVRAPQVYVLPPPEEEMTKKQVTLTC |
| | | MVTDEMPEDIYVEWTNNGKTELNYKNTEPVLDSDGSYFMYSKLRVEKKNWVER |
| | | NSYSCSVVHEGLHNHHTTKSFSRTPGK |
| FIT-Ig | SEQ ID NO: | EVQLVESGGGLVQPGRSLKLSCAASGFTFSDFYMAWVRQAPKKGLEWVASISY |
| Construct #2 | 546 | EGSSTYYGDSVMGRFTISRDNAKSTLYLQMNSLRSEDTATYYCARQREANWED |
| | | WGQGVMVTVSSAKTTAPSVYPLAPVCGDTTGSSVTLGCLVKGYFPEPVTLTWN |
| | | SGSLSSGVHTFPAVLQSDLYTLSSSVTVTSSTWPSQSITCNVAHPASSTKVDK |
| | | KI |
| FIT-Ig | SEQ ID NO: | DIVMTQSPSSLAVSPGEKVTMTCKSSQSLYYSGVKENLLAWYQQKPGQSPKLL |
| Construct #3 | 547 | IYYASIRFTGVPDRFTGSGSGTDYTLTITSVQAEDMGQYFCQQGINNPLTFGD |
| | | GTKLEIKRTDAAPTVSIFPPSSEQLTSGGASVVCFLNNFYPKDINVKWKIDGS |
| | | ERQNGVLNSWTDQDSKDSTYSMSSTLTLTKDEYERHNSYTCEATHKTSTSPIV |
| | | KSFNRNEC |

hIgG1 FIT-Ig bispecific 2b

| | | |
|---|---|---|
| Antibody A | anti-PD-L1 | |
| | 1D05 | |
| Antibody B | anti-ICOS | |
| | STIM001 | |
| FIT-Ig | SEQ ID NO: | DIVMTQSPSSLAVSPGEKVTMTCKSSQSLYYSGVKENLLAWYQQKPGQSPKLL |
| Construct #1 | 548 | IYYASIRFTGVPDRFTGSGSGTDYTLTITSVQAEDMGQYFCQQGINNPLTFGD |
| | | GTKLEIKRTDAAPTVSIFPPSSEQLTSGGASVVCFLNNFYPKDINVKWKIDGS |
| | | ERQNGVLNSWTDQDSKDSTYSMSSTLTLTKDEYERHNSYTCEATHKTSTSPIV |
| | | KSFNRNECEVQLVESGGGLVQPGRSLKLSCAASGFTFSDFYMAWVRQAPKKGL |
| | | EWVASISYEGSSTYYGDSVMGRFTISRDNAKSTLYLQMNSLRSEDTATYYCAR |
| | | QREANWEDWGQGVMVTVSSAKTTAPSVYPLAPVCGDTTGSSVTLGCLVKGYFP |
| | | EPVTLTWNSGSLSSGVHTFPAVLQSDLYTLSSSVTVTSSTWPSQSITCNVAHP |
| | | ASSTKVDKKIEPRGPTIKPCPPCKCPAPNLLGGPSVFIFPPKIKDVLMISLSP |
| | | IVTCVVVDVSEDDPDVQISWFVNNVEVHTAQTQTHREDYNSTLRVVSALPIQH |
| | | QDWMSGKEFKCKVNNKDLPAPIERTISKPKGSVRAPQVYVLPPPEEEMTKKQV |
| | | TLTCMVTDEMPEDIYVEWTNNGKTELNYKNTEPVLDSDGSYFMYSKLRVEKKN |
| | | WVERNSYSCSVVHEGLHNHHTTKSFSRTPGK |
| FIT-Ig | SEQ ID NO: | EVQLVESGGGLTQPGKSLKLSCEASGFTFSSFTMHWVRQSPGKGLEWVAFIRS |
| Construct #2 | 549 | GSGIVFYADAVRGRFTISRDNAKNLLFLQMNDLKSEDTAMYYCARRPLGHNTE |
| | | DSWGQGTLVTVSSAKTTAPSVYPLAPVCGDTTGSSVTLGCLVKGYFPEPVTLT |
| | | WNSGSLSSGVHTFPAVLQSDLYTLSSSVTVISSTWPSQSITCNVAHPASSTKV |
| | | DKKI |
| FIT-Ig | SEQ ID NO: | DIQMTQSPASLSASLGETVTIQCRASEDIYSGLAWFQQKPGKSPQLLIYGASS |
| Construct #3 | 550 | LQDGVPSRFSGSGSGTQYSLKISSMQTEDEGVYFCQQGLKYPPTFGSGTKLEI |
| | | KRTDAAPTVSIFPPSSEQLTSGGASVVCFLNNFYPKDINVKWKIDGSERQNGV |
| | | LNSWTDQDSKDSTYSMSSTLTLTKDEYERHNSYTCEATHKTSTSPIVKSFNRN |
| | | EC |

hIgG1 FIT-Ig bispecific 3a

| | | |
|---|---|---|
| Antibody A | anti-ICOS | |
| | STIM003 | |
| Antibody B | anti-PD-L1 | |
| | 1D05 | |
| FIT-Ig | SEQ ID NO: | DIQMTQSPASLSASLGETVTIQCRASEDIYSGLAWFQQKPGKSPQLLIYGASS |
| Construct #1 | 551 | LQDGVPSRFSGSGSGTQYSLKISSMQTEDEGVYFCQQGLKYPPTFGSGTKLEI |
| | | KRTVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNRG |
| | | ECEVQLVESGGGLTQPGKSLKLSCEASGFTFSSFTMHWVRQSPGKGLEWVAFI |
| | | RSGSGIVFYADAVRGRFTISRDNAKNLLFLQMNDLKSEDTAMYYCARRPLGHN |
| | | TFDSWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSN |
| | | TKVDKKVEPKSCDKTHTCPPNLLGGPSVFIFPPKIKDVLMISLSPIVTCVVVD |
| | | VSEDDPDVQISWFVNNVEVHTAQTQTHREDYNSTLRVVSALPIQHQDWMSGKE |
| | | FKCKVNNKDLPAPIERTISKPKGSVRAPQVYVLPPPEEEMTKKQVTLTCMVTD |
| | | FMPEDIYVEWTNNGKTELNYKNTEPVLDSDGSYFMYSKLRVEKKNWVERNSYS |
| | | CSVVHEGLHNHHTTKSFSRTPGK |
| FIT-Ig | SEQ ID NO: | EVQLVESGGGLVQPGRSLKLSCAASGFTFSDFYMAWVRQAPKKGLEWVASISY |
| Construct #2 | 552 | EGSSTYYGDSVMGRFTISRDNAKSTLYLQMNSLRSEDTATYYCARQREANWED |
| | | WGQGVMVTVSSASTKGPSVFPLAPSSKSTSGGTAALGCLVKDYFPEPVTVSWN |
| | | SGALTSGVHTFPAVLQSSGLYSLSSVVTVPSSSLGTQTYICNVNHKPSNTKVD |
| | | KKV |
| FIT-Ig | SEQ ID NO: | DIVMTQSPSSLAVSPGEKVTMTCKSSQSLYYSGVKENLLAWYQQKPGQSPKLL |
| Construct #3 | 553 | IYYASIRFTGVPDRFTGSGSGTDYTLTITSVQAEDMGQYFCQQGINNPLTFGD |
| | | GTKLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGEC |

hIgG1 FIT-Ig bispecific 3b

| | | |
|---|---|---|
| Antibody A | anti-PD-L1 | |
| | 1D05 | |
| Antibody B | anti-ICOS | |
| | STIM003 | |
| FIT-Ig | SEQ ID NO: | DIVMTQSPSSLAVSPGEKVTMTCKSSQSLYYSGVKENLLAWYQQKPGQSPKLL |
| Construct #1 | 554 | IYYASIRFTGVPDRFTGSGSGTDYTLTITSVQAEDMGQYFCQQGINNPLTFGD |
| | | GTKLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGECEVQLVESGGGLVQPGRSLKLSCAASGFTFSDFYMAWVRQAPKKGL |
| | | EWVASISYEGSSTYYGDSVMGRFTISRDNAKSTLYLQMNSLRSEDTATYYCAR |
| | | QREANWEDWGQGVMVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVNH |
| | | KPSNTKVDKKVEPKSCDKTHTCPPNLLGGPSVFIFPPKIKDVLMISLSPIVTC |
| | | VVVDVSEDDPDVQISWFVNNVEVHTAQTQTHREDYNSTLRVVSALPIQHQDWM |
| | | SGKEFKCKVNNKDLPAPIERTISKPKGSVRAPQVYVLPPPEEEMTKKQVTLTC |
| | | MVTDEMPEDIYVEWTNNGKTELNYKNTEPVLDSDGSYFMYSKLRVEKKNWVER |
| | | NSYSCSVVHEGLHNHHTTKSFSRTPGK |
| FIT-Ig | SEQ ID NO: | EVQLVESGGGLTQPGKSLKLSCEASGFTFSSFTMHWVRQSPGKGLEWVAFIRS |
| Construct #2 | 555 | GSGIVEYADAVRGRFTISRDNAKNLLFLQMNDLKSEDTAMYYCARRPLGHNTF |
| | | DSWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTK |
| | | VDKKV |
| FIT-Ig | SEQ ID NO: | DIQMTQSPASLSASLGETVTIQCRASEDIYSGLAWFQQKPGKSPQLLIYGASS |
| Construct #3 | 556 | LQDGVPSRFSGSGSGTQYSLKISSMQTEDEGVYFCQQGLKYPPTFGSGTKLEI |
| | | KRTVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNRG |
| | | EC |

hIgG1 FIT-Ig bispecific 4a

| | | |
|---|---|---|
| Antibody A | anti-ICOS | |
| | STIM001 | |
| Antibody B | anti-PD-L1 | |
| | 84G09 | |
| FIT-Ig | SEQ ID NO: | DIQMTQSPASLSASLGETVTIQCRASEDIYSGLAWFQQKPGKSPQLLIYGASS |
| Construct #1 | 557 | LQDGVPSRFSGSGSGTQYSLKISSMQTEDEGVYFCQQGLKYPPTFGSGTKLEI |
| | | KRTVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNRG |
| | | ECEVQLVESGGGLTQPGKSLKLSCEASGFTFSSFTMHWVRQSPGKGLEWVAFI |
| | | RSGSGIVFYADAVRGRFTISRDNAKNLLFLQMNDLKSEDTAMYYCARRPLGHN |
| | | TFDSWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSN |
| | | TKVDKKVEPKSCDKTHTCPPNLLGGPSVFIFPPKIKDVLMISLSPIVTCVVVD |
| | | VSEDDPDVQISWFVNNVEVHTAQTQTHREDYNSTLRVVSALPIQHQDWMSGKE |
| | | FKCKVNNKDLPAPIERTISKPKGSVRAPQVYVLPPPEEEMTKKQVTLTCMVTD |
| | | FMPEDIYVEWTNNGKTELNYKNTEPVLDSDGSYFMYSKLRVEKKNWVERNSYS |
| | | CSVVHEGLHNHHTTKSFSRTPGK |
| FIT-Ig | SEQ ID NO: | EVQLVESGGGLVQPGRSLKLSCAASGFTFSDFYMAWVRQAPKKGLEWVASISY |
| Construct #2 | 558 | EGSSTYYGDSVMGRFTISRDNAKSTLYLQMNSLRSEDTATYYCARQREANWED |
| | | WGQGVMVTVSSASTKGPSVFPLAPSSKSTSGGTAALGCLVKDYFPEPVTVSWN |
| | | SGALTSGVHTFPAVLQSSGLYSLSSVVTVPSSSLGTQTYICNVNHKPSNTKVD |
| | | KKV |
| FIT-Ig | SEQ ID NO: | DIVMTQSPSSLAVSPGEKVTMTCKSSQSLYYSGVKENLLAWYQQKPGQSPKLL |
| Construct #3 | 559 | IYYASIRFTGVPDRFTGSGSGTDYTLTITSVQAEDMGQYFCQQGINNPLTFGD |
| | | GTKLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGEC |

hIgG1 FIT-Ig bispecific 4b

| | | |
|---|---|---|
| Antibody A | anti-PD-L1 | |
| | 84G09 | |
| Antibody B | anti-ICOS | |
| | STIM001 | |
| FIT-Ig | SEQ ID NO: | DIVMTQSPSSLAVSPGEKVTMTCKSSQSLYYSGVKENLLAWYQQKPGQSPKLL |
| Construct #1 | 560 | IYYASIRFTGVPDRFTGSGSGTDYTLTITSVQAEDMGQYFCQQGINNPLTFGD |
| | | GTKLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGECEVQLVESGGGLVQPGRSLKLSCAASGFTFSDFYMAWVRQAPKKGL |
| | | EWVASISYEGSSTYYGDSVMGRFTISRDNAKSTLYLQMNSLRSEDTATYYCAR |
| | | QREANWEDWGQGVMVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVNH |
| | | KPSNTKVDKKVEPKSCDKTHTCPPNLLGGPSVFIFPPKIKDVLMISLSPIVTC |
| | | VVVDVSEDDPDVQISWFVNNVEVHTAQTQTHREDYNSTLRVVSALPIQHQDWM |
| | | SGKEFKCKVNNKDLPAPIERTISKPKGSVRAPQVYVLPPPEEEMTKKQVTLTC |
| | | MVTDEMPEDIYVEWTNNGKTELNYKNTEPVLDSDGSYFMYSKLRVEKKNWVER |
| | | NSYSCSVVHEGLHNHHTTKSFSRTPGK |
| FIT-Ig | SEQ ID NO: | EVQLVESGGGLTQPGKSLKLSCEASGFTFSSFTMHWVRQSPGKGLEWVAFIRS |
| Construct #2 | 561 | GSGIVFYADAVRGRFTISRDNAKNLLFLQMNDLKSEDTAMYYCARRPLGHNTF |
| | | DSWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTK |
| | | VDKKV |
| FIT-Ig | SEQ ID NO: | DIQMTQSPASLSASLGETVTIQCRASEDIYSGLAWFQQKPGKSPQLLIYGASS |
| Construct #3 | 562 | LQDGVPSRFSGSGSGTQYSLKISSMQTEDEGVYFCQQGLKYPPTFGSGTKLEI |
| | | KRTVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNRG |
| | | EC |

TABLE S4
Sequences of antibody heavy chain variable regions obtained from additional clones
CDRs are defined according to IMGT.

| CLONE ID | VH_NUCLEOTIDE_SEQUENCE | VH_AMINO_ACID_SEQ | HCDR1 | HCDR2 | HCDR3 |
|---|---|---|---|---|---|
| CL-61091 | CAGGTTCAACTGATGCAGTCTGGAACTGAGGTGAAGAAGCCTGGG | QVQLMQSGTEVKKPGASV | GYTFTTYG | ISAYSGDT | ARSSGWPHHYGMDV |
| | GCCTCAGTGAAGGTCTCCTGCAAGACTTCTGGTTACACCTTTACC | KVSCKTSGYTFTTYGITW | SEQ ID | SEQ ID NO: | SEQ ID NO: 567 |
| | ACCTATGGTATCACTTGGGTGCGACAGGCCCCTGGACAAGGGCTT | VRQAPGQGLEWMGWISAY | NO: 565 | 566 | |
| | GAGTGGATGGGATGGATCAGCGCTTACAGTGGTGACACAGACTAT | SGDTDYAQKFQGRVTVTT | | | |
| | GCACAGAAGTTCCAGGGCAGAGTCACCGTGACAACAGACACATCC | DTSTNTAYMELRSLKSDD | | | |
| | ACGAACACAGCCTACATGGAGTTGAGGAGCCTGAAATCTGACGAC | TAVYYCARSSGWPHHYGM | | | |
| | ACGGCCGTGTATTATTGTGCGAGAAGTAGTGGCTGGCCCCACCAC | DVWGQGTTVTVSS | | | |
| | TACGGTATGGACGTCTGGGGCCAAGGGACCACGGTCACCGTCTCC | SEQ ID NO: 564 | | | |
| | TCAG | | | | |
| | SEQ ID NO: 563 | | | | |
| CL-64536 | CAGGTTCAACTGGTGCAGTCTGGAGGTGAGGTGAAAAAGCCTGGG | QVQLVQSGGEVKKPGASV | GYTFTSYG | ISAYNGNT | ARSTSYYGSGTLYGMDV |
| | GCCTCAGTGAAGGTCTCCTGCAAGGCTTCTGGTTACACCTTTACC | KVSCKASGYTFTSYGESW | SEQ ID | SEQ ID NO: | SEQ ID NO: 570 |
| | AGCTATGGTTTCAGCTGGGTGCGACAGGCCCCTGGACAAGGACTA | VRQAPGQGLEWMGWISAY | NO: 377 | 378 | |
| | GAGTGGATGGGATGGATCAGCGCTTACAATGGTAACACAAACTAT | NGNTNYAQKLQGRVSMTT | | | |
| | GCACAGAAGCTCCAGGGCAGAGTCTCCATGACCACAGACACATCC | DTSTSTAYMELRSLRSDD | | | |
| | ACGAGCACAGCCTACATGGAGCTGAGGAGCTTGAGATCTGACGAC | TAVYFCARSTSYYGSGTL | | | |
| | ACGGCCGTGTATTTCTGTGCGCGATCTACGTCTTACTATGGTTCG | YGMDVWGQGTTVTVSS | | | |
| | GGGACCCTATACGGTATGGACGTCTGGGGCCAAGGGACCACGGTC | SEQ ID NO: 569 | | | |
| | ACCGTCTCCTCAG | | | | |
| | SEQ ID NO: 568 | | | | |
| CL-64837 | CAGGTTCAACTGGTGCAGTCTGGAGGTGAGGTGAAGAAGCCTGGG | QVQLVQSGGEVKKPGASV | GYTFTSYG | ISAYNGNT | ARSTSYYGSGTLYGMDV |
| | GCCTCAGTGAAGGTCTCCTGCAAGGCTTCTGGTTACACCTTTACC | KVSCKASGYTFTSYGFSW | SEQ ID | SEQ ID NO: | SEQ ID NO: 570 |
| | AGCTATGGTTTCAGCTGGGTGCGACAGGCCCCTGGACAAGGACTA | VRQAPGQGLEWMGWISAY | NO: 377 | 378 | |
| | GAGTGGATGGGATGGATCAGCGCTTACAATGGTAACACAAACTAT | NGNTNYAQKLQGRVSMTT | | | |
| | GCACAGAAGCTCCAGGGCAGAGTCTCCATGACCACAGACACATCC | DTSTSTAYMELRSLRSDD | | | |
| | ACGAGCACAGCCTACATGGAGCTGAGGAGCTTGAGATCTGACGAC | TAVYYCARSTSYYGSGTL | | | |
| | ACGGCCGTGTATTACTGTGCGCGATCTACGTCTTACTATGGTTCG | YGMDVWGQGTTVTVSS | | | |
| | GGGACCCTCTACGGTATGGACGTCTGGGGCCAAGGGACCACGGTC | SEQ ID NO: 572 | | | |
| | ACCGTCTCCTCAG | | | | |
| | SEQ ID NO: 571 | | | | |
| CL-64841 | CAGGTTCAACTGGTGCAGTCTGGAGGTGAGGTGAAAAAGCCTGGG | QVQLVQSGGEVKKPGASV | GYTFTSYG | ISAYNGNT | ARSTSYYGSGTLYGMDV |
| | GCCTCAGTGAAGGTCTCCTGCAAGGCTTCTGGTTACACCTTTACC | KVSCKASGYTFTSYGESW | SEQ ID | SEQ ID NO: | SEQ ID NO: 570 |
| | AGCTATGGTTTCAGCTGGGTGCGACAGGCCCCTGGACAAGGACTA | VRQAPGQGLEWMGWISAY | NO: 377 | 378 | |
| | GAGTGGATGGGATGGATCAGCGCTTACAATGGTAACACAAACTAT | NGNTNYAQKLQGRVSMTT | | | |
| | GCACAGAAGCTCCAGGGCAGAGTCTCCATGACCACAGACACATCC | DTSTSTAYMELRSLRSDD | | | |
| | ACGAGCACAGCCTACATGGAGCTGAGGAGCTTGAGATCTGACGAC | TAVYFCARSTSYYGSGTL | | | |
| | ACGGCCGTGTATTTCTGTGCGCGATCTACGTCTTACTATGGTTCG | YGMDVWGQGTTVTVSS | | | |
| | GGGACCCTATACGGTATGGACGTCTGGGGCCAAGGGACCACGGTC | SEQ ID NO: 574 | | | |
| | ACCGTCTCCTCAG | | | | |
| | SEQ ID NO: 573 | | | | |
| CL-64912 | CAGGTTCAACTGGTGCAGTCTGGAGGTGAGGTGAAAAAGCCTCGG | QVQLVQSGGEVKKPRASV | GYTFTSYV | ISGYNGNT | ARSTSYYGAGTLYGMDV |
| | GCCTCAGTGAAGGTCTCCTGCAAGGCTTCTGGTTACACCTTTACC | KVSCKASGYTFTSYVFSW | SEQ ID | SEQ ID NO: | SEQ ID NO: 579 |
| | AGCTATGTGTTCAGCTGGGTGCGACATGCCGCTGGACAAGGACTA | VRHAAGQGLEWMGWISGY | NO: 577 | 578 | |
| | GAGTGGATGGGATGGATCAGCGGTTACAATGGTAACACAAACTAT | NGNTNYAQKLQCGVSMTA | | | |
| | GCACAGAAGCTCCAGTGCGGAGTCTCGATGACCGCAGACACATCC | DTSTSTAYMELRSLRSDD | | | |
| | ACGAGCACAGCCTACATGGAGCTGAGGAGCTTGAGATCTGACGAC | TAVYFCARSTSYYGAGTL | | | |
| | ACGGCCGTGTATTTCTGTGCGCGATCTACGTCTTACTATGGTGCG | YGMDVWGQGTTVTVSS | | | |
| | GGGACCCTATACGGTATGGACGTCTGGGGCCAAGGGACCACGGTC | SEQ ID NO: 576 | | | |
| | ACCGTCTCCTCAG | | | | |
| | SEQ ID NO: 575 | | | | |
| CL-71642 | GAGGTGCAGCTGGTGGAGTCTGGGGGAGGTGTGGTACGGCCTGGG | EVQLVESGGGVVRPGGSL | GFTFDDYG | INWNGGST | AADYYGSGSYYNVPFDY |
| | GGGTCCCTGAGACTCTCCTGTGCAGCCTCTGGATTCACCTTTGAT | RLSCAASGFTFDDYGMSW | SEQ ID | SEQ ID NO: | SEQ ID NO: 584 |
| | GATTATGGCATGAGCTGGGTCCGCCAAGCTCCAGGGAAGGGGCTG | VRQAPGKGLEWVSGINWN | NO: 582 | 583 | |
| | GAGTGGGTCTCTGGTATTAATTGGAATGGTGGTAGCACAGGTTAT | GGSTGYADSVKGRFTISR | | | |
| | GCAGACTCTGTGAAGGGCCGATTCACCATCTCCAGAGACAACGCC | DNAKNSLYLQMNSLRAED | | | |
| | AAGAACTCCCTGTATCTGCAAATGAACAGTCTGAGAGCCGAGGAC | TALYYCAADYYGSGSYYN | | | |
| | ACGGCCTTGTATTACTGTGCGGCCGATTACTATGGTTCGGGGAGT | VPFDYWGQGTLVTVSS | | | |
| | TATTATAACGTCCCCTTTGACTACTGGGGCCAGGGAACCCTGGTC | SEQ ID NO: 581 | | | |
| | ACCGTCTCCTCAG | | | | |
| | SEQ ID NO: 580 | | | | |
| CL-74570 | GAGGTGCAGCTGGTGGAGTCTGGGGGAGGTGTGATACGGCCTGGG | EVQLVESGGGVIRPGGSL | GFTFDDYG | INWIGDNT | ARDYFGSGSYYNVPFDY |
| | GGGTCCCTGAGACTCTCCTGTGCAGCCTCTGGATTCACCTTTGAT | RLSCAASGFTFDDYGMSW | SEQ ID | SEQ ID NO: | SEQ ID NO: 588 |
| | GATTATGGCATGAGCTGGGTCCGCCAAGCTCCAGGGAAGGGGCTG | VRQAPGKGLEWVSGINWI | NO: 582 | 587 | |
| | GAGTGGGTCTCTGGTATTAATTGGATTGGTGATAACACAGATTAT | GDNTDYADSVKGRFTISR | | | |
| | GCAGACTCTGTGAAGGGCCGATTCACCATCTCCAGAGACAACGCC | DNAKNSLYLQMNSLRAED | | | |
| | AAGAACTCCCTATATCTGCAAATGAACAGTCTGAGAGCCGAGGAC | TALYYCARDYFGSGSYYN | | | |
| | ACGGCCTTGTATTACTGTGCGAGAGATTACTTTGGTTCGGGGAGT | VPFDYWGQGTLVTVSS | | | |
| | TATTATAACGTTCCCTTTGACTACTGGGGCCAGGGAACCCTGGTC | SEQ ID NO: 586 | | | |
| | ACCGTCTCCTCAG | | | | |
| | SEQ ID NO: 585 | | | | |

TABLE S5
Sequences of antibody light chain variable regions obtained from additional clones
N terminal E and 5′ nucleotide additions in CL-71642 are shown in bold. These were not recovered in sequencing but were determined to be
present in the sequence by comparison against the related clones as shown in FIG. 11. CDRs are defined according to IMGT.

| CLONE ID | VL_NUCLEOTIDE_SEQUENCE | VL_AMINO_ACID_SEQ | LCDR1 | LCDR2 | LCDR3 |
|---|---|---|---|---|---|
| CL-61091 | GATATTGTGATGACTCAGTCTCCACTCTCCCTGCCCGTCACCCCT | DIVMTQSPLSLPVTPGEPA | QSLLHSNGFNY | LVS | MQALQTPLT |
| | GGAGAGCCGGCCTCCATCTCCTGCAGGTCTAGTCAGAGCCTCCTG | SISCRSSQSLLHSNGFNYF | SEQ ID NO: | SEQ ID | SEQ ID NO: 593 |
| | CATAGTAATGGATTCAACTATTTCGATTGGTACCTGCAGAAGCCA | DWYLQKPGQSPQLLIFLVS | 591 | NO: | |
| | GGACAGTCTCCACAGCTCCTGATCTTTTTGGTTTCTAATCGGGCC | NRASGVPDRFSGSGSGTDF | | 592 | |
| | TCCGGGGTCCCTGACAGGTTCAGTGGCAGTGGATCAGGCACAGAT | TLKISRVEAEDVGIYYCMQ | | | |
| | TTTACACTGAAAATCAGCAGAGTGGAGGCTGAGGATGTTGGGATT | ALQTPLTFGGGTKVEIK | | | |
| | TATTACTGCATGCAAGCTCTACAAACTCCGCTCACTTTCGGCGGA | SEQ ID NO: 590 | | | |
| | GGGACCAAGGTGGAGATCAAAC | | | | |
| | SEQ ID NO: 589 | | | | |
| CL-64536 | GATATTGTGATGACTCAGTCTCCACTCTCCCTGCCCGTCACCCCT | DIVMTQSPLSLPVTPGEPA | QSLLHSNGYNC | LGS | MQALQTPCS |
| | GGAGAGCCGGCCTCCATCTCCTGCAGGTCTAGTCAGAGCCTCCTG | SISCRSSQSLLHSNGYNCL | SEQ ID NO: | SEQ ID | SEQ ID NO: 400 |
| | CATAGTAATGGATACAACTGTTTGGATTGGTACCTGCAGAAGCCA | DWYLQKPGQSPQLLIYLGS | 596 | NO: | |
| | GGGCAGTCTCCACAGCTCCTGATCTATTTGGGTTCTACTCGGGCC | TRASGFPDRFSGSGSGTDF | | 371 | |
| | TCCGGGTTCCCTGACAGGTTCAGTGGCAGTGGATCAGGCACAGAT | TLKISRVEAEDVGVYYCMQ | | | |
| | TTTACACTGAAAATCAGCAGAGTGGAGGCTGAGGATGTTGGGGTT | ALQTPCSFGQGTKLEIK | | | |
| | TATTACTGCATGCAAGCTCTACAAACTCCGTGCAGTTTTGGCCAG | SEQ ID NO: 595 | | | |
| | GGGACCAAGCTGGAGATCAAAC | | | | |
| | SEQ ID NO: 594 | | | | |
| CL-64837 | GATATTGTGATGACTCAGTCTCCACTCTCCCTGCCCGTCACCCCT | DIVMTQSPLSLPVTPGEPA | QSLLHSNGYNC | LGS | MQALQTPCS |
| | GGAGAGCCGGCCTCCATCTCCTGCAGGTCTAGTCAGAGCCTCCTG | SISCRSSQSLLHSNGYNCL | SEQ ID NO: | SEQ ID | SEQ ID NO: 400 |
| | CATAGTAATGGATACAACTGTTTGGATTGGTACCTGCAGAAGCCA | DWYLQKPGQSPQLLIYLGS | 596 | NO: | |
| | GGGCAGTCTCCACAGCTCCTGATCTATTTGGGTTCTACTCGGGCC | TRASGFPDRFSGSGSGTDF | | 371 | |
| | TCCGGGTTCCCTGACAGGTTCAGTGGCAGTGGATCAGGCACAGAT | TLKISRVEAEDVGVYYCMQ | | | |
| | TTTACACTGAAAATCAGCAGAGTGGAGGCTGAGGATGTTGGGGTT | ALQTPCSFGQGTKLEIK | | | |
| | TATTACTGCATGCAAGCTCTACAAACTCCGTGCAGTTTTGGCCAG | SEQ ID NO: 598 | | | |
| | GGGACCAAGCTGGAGATCAAAC | | | | |
| | SEQ ID NO: 597 | | | | |
| CL-64841 | GATATTGTGATGACTCAGTCTCCACTCTCCCTGCCCGTCACCCCT | DIVMTQSPLSLPVTPGEPA | QSLLHSNGYNC | LGS | MQALQTPCS |
| | GGAGAGCCGGCCTCCATCTCCTGCAGGTCTAGTCAGAGCCTCCTG | SISCRSSQSLLHSNGYNCL | SEQ ID NO: | SEQ ID | SEQ ID NO: 400 |
| | CATAGTAATGGATACAACTGTTTGGATTGGTACCTGCAGAAGCCA | DWYLQKPGQSPQLLIYLGS | 596 | NO: | |
| | GGGCAGTCTCCACAGCTCCTGATCTATTTGGGTTCTACTCGGGCC | TRASGFPDRFSGSGSGTDS | | 371 | |
| | TCCGGGTTCCCTGACAGGTTCAGTGGCAGTGGATCAGGCACAGAT | TLKISRVEAEDVGVYYCMQ | | | |
| | TCTACACTGAAAATCAGCAGAGTGGAGGCTGAGGATGTTGGGGTT | ALQTPCSFGQGTKLEIK | | | |
| | TATTACTGCATGCAAGCTCTACAAACTCCGTGCAGTTTTGGCCAG | SEQ ID NO: 600 | | | |
| | GGGACCAAGCTGGAGATCAAAC | | | | |
| | SEQ ID NO: 599 | | | | |
| CL-64912 | GATATTGTGATGACTCAGTCTCCACTCTCCCTGCCCGTCACCCCT | DIVMTQSPLSLPVTPGEPA | QSLLHSNGYNC | LGS | MQALQTPCS |
| | GGAGAGCCGGCCTCCATCTCCTGCAGGTCTAGTCAGAGCCTCCTG | SISCRSSQSLLHSNGYNCL | SEQ ID NO: | SEQ ID | SEQ ID NO: 400 |
| | CATAGTAATGGATACAACTGTTTGGATTGGTACCTGCAGAAGCCA | DWYLQKPGQSPQLLIYLGS | 596 | NO: | |
| | GGGCAGTCTCCACAGCTCCTGATCTATTTGGGTTCTACTCGGGCC | TRASGFPDRFSGSGSGTDE | | 371 | |
| | TCCGGGTTCCCTGACAGGTTCAGTGGCAGTGGATCAGGCACAGAT | TLKISRVEAEDVGVYYCMQ | | | |
| | TTTACACTGAAAATCAGCAGAGTGGAGGCTGAGGATGTTGGGGTT | ALQTPCSFGQGTKLEIK | | | |
| | TATTACTGCATGCAAGCTCTACAAACTCCGTGCAGTTTTGGCCAG | SEQ ID NO: 602 | | | |
| | GGGACCAAGCTGGAGATCAAAC | | | | |
| | SEQ ID NO: 601 | | | | |
| CL-71642 | GAAATTGTGTTGACGCAGTCTCCAGGCACCCTGTCTTTGTCTCCA | EIVLTQSPGTLSLSPGERA | QSVSSSY | GAS | QQYGSSPFT |
| | GGGGAAAGAGCCACCCTCTCCTGCAGGGCCAGTCAGAGTGTTAGC | TLSCRASQSVSSSYLAWYQ | SEQ ID NO: | SEQ ID | SEQ ID NO: 605 |
| | AGCAGCTACTTAGCCTGGTACCAGCAGAAACCTGGCCAGGCTCCC | QKPGQAPRLLIYGASSRAT | 426 | NO: | |
| | AGGCTCCTCATCTATGGTGCATCCAGCAGGGCCACTGGCATCCCA | GIPDRFSGSGSGTDFTLTI | | 413 | |
| | GACAGGTTCAGTGGCAGTGGGTCTGGGACAGACTTCACTCTCACC | SRLEPEDFAVYYCQQYGSS | | | |
| | ATCAGCAGACTGGAGCCTGAAGATTTTGCAGTGTATTACTGTCAG | PFTFGPGTKVDIK | | | |
| | CAGTATGGTAGCTCACCTTTCACTTTCGGCCCTGGGACCAAAGTG | SEQ ID NO: 604 | | | |
| | GATATCAAAC | | | | |
| | SEQ ID NO: 603 | | | | |
| CL-74570 | GAAATTGTGTTGACGCAGTCTCCAGGCACCCTGTCTTTGTCTCCA | EIVLTQSPGTLSLSPGERA | QSVSSSY | GAS | HQYGNSPFT |
| | GGGGAAAGAGCCACCCTCTCCTGCAGGGCCAGTCAGAGTGTTAGC | TLSCRASQSVSSSYLAWYQ | SEQ ID NO: | SEQ ID | SEQ ID NO: 608 |
| | AGCAGCTACTTAGCCTGGTACCAGCAGAAACCTGGCCAGGCTCCC | QKPGQAPRLLIYGASSRAT | 426 | NO: | |
| | AGGCTCCTCATCTATGGTGCATCCAGCAGGGCCACTGGCATCCCA | GIPDRFSGSGSGTDFTLTI | | 413 | |
| | GACAGGTTCAGTGGCAGTGGGTCTGGGACAGACTTCACTCTCACC | SRLEPEDFAVYYCHQYGNS | | | |
| | ATCAGCAGACTGGAACCTGAAGATTTTGCAGTATATTACTGTCAC | PFTFGPGTKVDIK | | | |
| | CAGTATGGTAATTCACCATTCACTTTCGGCCCTGGGACCAAAGTG | SEQ ID NO: 607 | | | |
| | GATATCAAAC | | | | |
| | SEQ ID NO: 606 | | | | |

### CLAUSES

1. A method of treating cancer in a patient, wherein the patient has a PD-L1 negative tumour or a tumour with low PD-L1 expression, comprising administering to the patient a modulator of ICOS.

2. The method of clause 1, comprising administering to the patient a modulator of ICOS and an inhibitor of PD-1.

3. A method of treating cancer in a patient who has previously received treatment for the cancer, wherein the previous treatment for the cancer was administration of a PD-L1 inhibitor and the patient did not respond to the previous treatment or ceased responding to the previous treatment, and wherein the patient has a PD-L1 negative tumour or a tumour with low PD-L1 expression, comprising administering to the patient a modulator of ICOS.

4. The method of clause 3, comprising administering to the patient a modulator of ICOS and an inhibitor of PD-1.

5. The method of any preceding clause, comprising determining the level of PD-L1 expression in a tumour sample from the patient, and if the tumour is PD-L1 negative or a PD-L1 low expression tumour, then administering to the patient an ICOS modulator.

6. The method of clause 5, comprising administering to the patient a modulator of ICOS and an inhibitor of PD-1.

7. The method of any one of clauses 2, 4, and 6, wherein the modulator of ICOS and an inhibitor of PD-L1 are administered simultaneously, separately or sequentially.

8. The method of any preceding clause, wherein the ICOS modulator is an ICOS agonist.

9. The method of any preceding clause, wherein the ICOS agonist is an agonistic anti-ICOS antibody.

10. The method of clause 9, wherein the anti-ICOS antibody is a bispecific antibody that specifically binds ICOS and PD-L1 or specifically binds ICOS and PD-1.

11. The method of clause 9, wherein the bispecific antibody is an ICOS agonist and a PD-L1 antagonist, or an ICOS agonist and a PD-1 antagonist.

12. The method of any preceding clause, wherein the PD-L1 inhibitor is an anti-PD-L1 binding molecule.

13. The method of any preceding clause, wherein the PD-L1 inhibitor is an anti-PD-L1 antibody or an anti-PD-1 antibody.

14. The method of any preceding clause, wherein the PD-L1 inhibitor inhibits the binding of PD-L1 to PD-1.

15. The method of any preceding clause, wherein the PD-L1 inhibitor is an antagonistic anti-PD-L1 antibody or an antagonistic anti-PD-1 antibody.

16. The method of any preceding clause, wherein the tumour cells are PD-L1 negative or exhibit low PD-L1 expression.

17. The method of any preceding clause, wherein the tumour comprises immune cells, and the immune cells are PD-L1 negative or exhibit low PD-L1 expression.

18. The method of clause 17, wherein the tumour cells are PD-L1 negative or exhibit low PD-L1 expression and the immune cells are PD-L1 negative or exhibit low PD-L1 expression.

19. The method of any preceding clause, wherein the cancer is associated with infectious agents.

20. The method of clause 19, wherein the cancer is a virally-induced cancer.

21. The method of clause 20, wherein the virus associated with the virally-induced cancer is selected from HPV (cervical cancer, oropharyngeal cancer), HBV, HCV, and EBV (Burkitts lymphomas, gastric cancer, Hodgkin's lymphoma, other EBV positive B cell lymphomas, nasopharyngeal carcinoma and post-transplant lymphoproliferative disease).

22. The method of clause 21, wherein the cancer is selected from the group consisting of head and neck squamous cell carcinoma, cervical cancer, anogenital cancer and oropharyngeal cancer.

23. The method of any preceding clause, wherein the tumour is HPV (Human papillomavirus) positive.

24. The method of any preceding clause, wherein the patient has undergone a test for an infection, optionally wherein the infection is selected from HPV, HBV, HCV, or EBV infection.

25. The method of clause 24, wherein the patient has undergone a test for HPV infection.

26. The method of any preceding clause, wherein the patient has an HPV infection or has had an HPV infection.

27. The method of any preceding clause, comprising the step of determining the HPV status of the patient and/or determining the HPV status of the tumour.

28. The method of any preceding clause, wherein the tumour cells are PD-L1 negative or exhibit low PD-L1 expression and the tumour is HPV (Human papillomavirus) positive.

29. The method of any preceding clause, wherein the patient has previously been administered a kinase inhibitor.

30. The method of any preceding clause, wherein the patient has previously received surgical treatment for the cancer (for example complete or partial tumour resection) and/or radiotherapy and/or chemotherapy.

31. The method of clause 30, wherein the chemotherapy comprises docetaxel, fluorouracil, cisplatin, paclitaxel and/or nab-paclitaxel.

32. The method of any preceding clause, wherein the cancer is or has been characterised as refractory to PD-L1 inhibitor treatment (for example refractory to anti-PD-L1 antibody or anti-PD-1 antibody monotherapy).

33. The method of clause 32, wherein the cancer is or has been characterised as refractory to PD-L1 inhibitor monotherapy treatment.

34. The method of clause 32, wherein the cancer is or has been characterised as refractory to treatment with a PD-L1 inhibitor as the sole immunotherapy agent.

35. The method of any one of clauses 32 to 34, wherein the cancer is or has been characterised as refractory to treatment with nivolumab.

36. The method of any preceding clause, wherein the patient has previously received treatment for the cancer.

37. The method of clause 36 wherein the previous treatment for the cancer was administration of a PD-L1 inhibitor (for example an anti-PD-L1 antibody or an anti-PD-1 antibody).

38. The method of clause 36, wherein the previous treatment for the cancer was PD-L1 inhibitor monotherapy.

39. The method of clause 36, wherein the previous treatment for the cancer was administration of a PD-L1 inhibitor as the sole immunotherapeutic agent.

40. The method of any preceding clause, wherein the PD-L1 expression status is determined by immunohistochemistry (IHC).

41. The method of clause 40, wherein the IHC is performed on a tumour sample.

42. The method of clause 41, wherein the tumour sample is tumour tissue sample or a sample of tumour cells.

43. The method of any preceding clause, wherein the tumour is a low PD-L1 expressing tumour when 25% or less of the tumour cells express PD-1.

44. The method of any preceding clause, wherein the tumour is a low PD-L1 expressing tumour when less than 20%, less than 15%, less than 10%, less than 5%, less that about 4%, less than about 3%, less than about 2% or less than about 1% of tumour cells express PD-1.

45. The method of any preceding clause, wherein 0% of tumour cells express PD-1.

46. The method of any preceding clause, wherein the tumour is a low PD-L1 expressing tumour when 25% or less of the tumour cells and tumour-associated immune cells express PD-1.

47. The method of any preceding clause, wherein the tumour is a low PD-L1 expressing tumour when less than 20%, less than 15%, less than 10%, less than 5%, less than about 5%, less that about 4%, less than about 3%, less than about 2% or less than about 1% of tumour cells and tumour-associated immune cells express PD-1.

48. The method of any preceding clause, wherein 0% of tumor cells and tumour-associated immune cells express PD-1.

49. The method of any one of clauses 43 to 48, wherein the percentage of PD-L1 expression is determined according to the following formula: (number of PD-L1 positive tumour cells in the tumour tissue sample or sample of tumour cells/total number of tumour cells in the tumour tissue sample or sample of tumour cells)×100.

50. The method of any one of clauses 43 to 48, wherein the percentage of PD-L1 expression is determined according to the following formula: (number of PD-L1 positive tumour cells and number of PD-L1 positive tumour-associated immune cells in the tumour tissue sample or sample of tumour cells/total number of tumour cells and tumour-associated immune in the tumour tissue sample or sample of tumour cells)×100.

51. The method of any preceding clause, wherein the tumour is a CD8+ tumour.

52. The method of clause 51, wherein the CD8 expression status is determined by immunohistochemistry (IHC).

53. The method of clause 51 or clause 52, wherein the at least 50% of the T-cells in the tumour are CD8+.

54. The method of clause 51 or clause 52 or clause 53, wherein the tumour tissue sample or sample of tumour cells comprises at least 190 cells CD8+ T-cells per mm².

55. The method of any preceding clause, wherein the tumour is a ICOS+ tumour.

56. The method of clause 55, wherein the ICOS expression status is determined by immunohistochemistry (IHC).

57. The method of clause 55 or clause 56, wherein the at least 50% of the immune cells in the tumour are ICOS+.

58. The method of any preceding clause, wherein the patient has an increased level of ICOS' immune cells (such as ICOS' regulatory T cells) following treatment with another therapeutic agent.

59. The method of clause 58, wherein the method comprises administering a therapeutic agent to the patient, determining that the patient has an increased level of ICOS' immune cells (such as ICOS' regulatory T cells) following the treatment with said agent, and administering a modulator of ICOS (for example an anti-ICOS antibody such as an agonistic anti-ICOS antibody) to the patient to reduce the level of ICOS' regulatory T cells.

60. The method of clause 58 or clause 59, wherein the therapeutic agent is IL-2 or an immunomodulatory antibody (e.g., anti-PDL-1, anti-PD-1 or anti-CTLA-4).

61. The method of any preceding clause, comprising administering a single dose of the ICOS modulator.

62. The method of any preceding clause, comprising administering a single dose of the ICOS modulator followed by multiple doses of the PD-L1 inhibitor.

63. The method of any preceding clause, wherein the ICOS modulator and the PD-L1 inhibitor are provided in separate compositions for administration.

64. The method of any preceding clause, wherein the ICOS modulator depletes ICOS+ immune cells, e.g. ICOS+ Treg cells.

65. The method of any preceding clause, wherein treatment results in reducing the size of the tumour.

66. The method of any preceding clause, wherein treatment inhibits tumour growth.

67. The method of any preceding clause, wherein treatment results in stable disease.

68. The method of any preceding clause, wherein treatment extends the survival of the patient and/or delays disease progression.

69. The method of any preceding clause, wherein the treatment depletes ICOS+ immune cells (such as ICOS+ regulatory T cells) in the tumour microenvironment.

70. The method of any preceding clause, where treatment increases the CD8+ to ICOS+ immune cell ratio (for example the CD8+ to ICOS+ regulatory T cell ration) in the tumour microenvironment.

71. The method of any preceding clause, wherein the ICOS modulator is an anti-ICOS antibody.

72. The method of clause 71, wherein the anti-ICOS antibody is any of the following antibodies, may comprise the VH and VL domains of any of the following antibodies or may comprise the HCDRs and/or LCDRs of any of the following antibodies:
a. KY1044;
b. anti-ICOS antibodies described in PCT/GB2017/052352 WO2018/029474 or U.S. Pat. No. 9,957,323, the contents of which are incorporated herein by reference (e.g., STIM001, STIM002, STIM002B, STIM003, STIM004, STIM005, STIM006, STIM007, STIM008 or STIM009);
c. anti-ICOS antibodies described in PCT/GB2018/053701 WO2019/122884 the contents of which are incorporated herein by reference (e.g., STIM017, STIM020, STIM021, STIM022, STIM023, STIM039, STIM040, STIM041, STIM042, STIM043, STIM044, STIM050, STIM051, STIM052, STIM053, STIM054, STIM055, STIM056, STIM057, STIM058, STIM059, STIM060, STIM061, STIM062, STIM063, STIM064, STIM065 or STIM066);
d. anti-ICOS/PD-L1 mAb2 bispecific antibodies described in PCT/GB2018/053698 WO2019/122882;
e. vopratelimab;
f. anti-ICOS antibodies described in WO2016/154177 or US2016/0304610 (e.g., 37A10S713, 7F12, 37A10, 35A9, 36E10, 16G10, 37A10S714, 37A10S715, 37A10S716, 37A10S717, 37A10S718, 16G10S71, 16G10S72, 16G10S73, 16G10S83, 35A9S79, 35A9S710 or 35A9S89);
g. anti-ICOS antibodies described in WO2016/120789 or US2016/0215059 (e.g., 422.2 H2L5);
h. antibody C398.4 or a humanised antibody thereof as described in WO2018/187613 or US2018/0289790, e.g., ICOS.33 IgG1f S267E, ICOS.4, ICOS34 G1f, ICOS35 G1f, 17C4, 9D5, 3E8, 1D7a, 1D7b or 2644 (for sequences see WO2018187613 Table 35), e.g., the antibody BMS-986226 in NCT03251924;
i. antibody JMAb 136, “136”, or any other antibody described in WO2010/056804;
j. antibody 314-8, the antibody produced from hybridoma CNCM I-4180, or any other anti-ICOS antibody described in WO2012/131004, WO2014/033327 or US2015/0239978;
k. antibody Icos145-1, the antibody produced by hybridoma CNCM I-4179, or any other antibody described in WO2012/131004, U.S. Pat. No. 9,376,493 or US2016/0264666;
l. antibody MIC-944 (from hybridoma DSMZ 2645), 9F3 (DSMZ 2646) or any other anti-ICOS antibody described in WO99/15553, U.S. Pat. Nos. 7,259,247, 7,132,099, 7,125,551, 7,306,800, 7,722,872, WO05/103086, US8,318.905 or U.S. Pat. No. 8,916,155;
m. anti-ICOS antibodies described in WO98/3821, U.S. Pat. No. 7,932,358B2, US2002/156242, U.S. Pat. Nos. 7,030,225, 7,045,615, 7,279,560, 7,226,909, 7,196,175, 7,932,358, 8,389,690, WO02/070010, U.S. Pat. Nos. 7,438,905, 7,438,905, WO01/87981, U.S. Pat. Nos. 6,803,039, 7,166,283, 7,988,965, WO01/15732, U.S. Pat. No. 7,465,445 or U.S. Pat. No. 7,998,478 (e.g., JMAb-124, JMAb-126, JMAb-127, JMAb-128, JMAb-135, JMAb-136, JMAb-137, JMAb-138, JMAb-139, JMAb-140 or JMAb-141, e.g., JMAb136);
n. anti-ICOS antibodies described in WO2014/08911;
o. anti-ICOS antibodies described in WO2012/174338;
p. anti-ICOS antibodies described in US2016/0145344;
q. anti-ICOS antibodies described in WO2011/020024, US2016/002336, US2016/024211 or U.S. Pat. No. 8,840,889;
r. anti-ICOS antibodies described in U.S. Pat. No. 8,497,244; or
s. antibody clone ISA-3 (eBioscience), clone SP98 (Novus Biologicals), clone 1 G1, clone 3G4 (Abnova Corporation), clone 669222 (R&D Systems), clone TQ09 (Creative Diagnostics), clone 2C7 (Deng et al. Hybridoma Hybridomics 2004), clone ISA-3 (eBioscience) or clone 17G9 (McAdam et al. J Immunol 2000).

73. The method of clause 71, wherein the anti-ICOS antibody is an antibody that binds the extracellular domain of human and/or mouse ICOS, wherein the antibody comprises a VH domain comprising an amino acid sequence having at least 95% sequence identity to the STIM003 VH domain SEQ ID NO: 408 and a VL domain comprising an amino acid sequence having at least 95% sequence identity to the STIM003 VL domain SEQ ID NO: 415.

74. The method of clause 73, wherein the VH domain comprises a set of heavy chain complementarity determining regions (HCDRs) HCDR1, HCDR2 and HCDR3, wherein
a. HCDR1 is the STIM003 HCDR1 having amino acid sequence SEQ ID NO: 405,
b. HCDR2 is the STIM003 HCDR2 having amino acid sequence SEQ ID NO: 406,
c. HCDR3 is the STIM003 HCDR3 having amino acid sequence SEQ ID NO: 407.

75. The method of clause 73 or clause 74, wherein the VL domain comprises a set of light chain complementarity determining regions (LCDRs) LCDR1, LCDR2 and LCDR3, wherein:
a. LCDR1 is the STIM003 LCDR1 having amino acid sequence SEQ ID NO: 412,
b. LCDR2 is the STIM003 LCDR2 having amino acid sequence SEQ ID NO: 413,
c. LCDR3 is the STIM003 LCDR3 having amino acid sequence SEQ ID NO: 414.

76. The method of clause 73, wherein the VH domain amino acid sequence is SEQ ID NO: 408 and/or wherein the VL domain amino acid sequence is SEQ ID NO: 415.

77. The method of clause 71, wherein the anti-ICOS antibody is an antibody that binds the extracellular domain of human and/or mouse ICOS, comprising
an antibody VH domain comprising complementarity determining regions (CDRs) HCDR1, HCDR2 and HCDR3, and
an antibody VL domain comprising complementarity determining regions LCDR1, LCDR2 and LCDR3, wherein
HCDR1 is the HCDR1 of STIM001, STIM002, STIM002-B, STIM003, STIM004, STIM005, STIM006, STIM007, STIM008 or STIM009, or comprises that HCDR1 with 1, 2, 3, 4 or 5 amino acid alterations,
HCDR2 is the HCDR2 of STIM001, STIM002, STIM002-B, STIM003, STIM004, STIM005, STIM006, STIM007, STIM008 or STIM009, or comprises that HCDR2 with 1, 2, 3, 4 or 5 amino acid alterations, and/or
HCDR3 is the HCDR3 of STIM001, STIM002, STIM002-B, STIM003, STIM004, STIM005, STIM006, STIM007, STIM008 or STIM009 or comprises that HCDR3 with 1, 2, 3, 4 or 5 amino acid alterations.

78. The method of clause 77, wherein the antibody heavy chain CDRs are those of STIM001, STIM002, STIM002-B, STIM003, STIM004, STIM005, STIM006, STIM007, STIM008 or STIM009 or comprise the STIM001, STIM002, STIM002-B, STIM003, STIM004, STIM005, STIM006, STIM007, STIM008 or STIM009 heavy chain CDRs with 1, 2, 3, 4 or 5 amino acid alterations.

79. The method of clause 78, wherein the antibody VH domain has the heavy chain CDRs of STIM003.

80. The method of clause 71, wherein the anti-ICOS antibody is an antibody binds the extracellular domain of human and/or mouse ICOS, comprising
an antibody VH domain comprising complementarity determining regions HCDR1, HCDR2 and HCDR3, and
an antibody VL domain comprising complementarity determining regions LCDR1, LCDR2 and LCDR3,
wherein LCDR1 is the LCDR1 of STIM001, STIM002, STIM002-B, STIM003, STIM004 STIM005, STIM006, STIM007, STIM008 or STIM009, or comprises that LCDR1 with 1, 2, 3, 4 or 5 amino acid alterations,
LCDR2 is the LCDR2 of STIM001, STIM002, STIM002-B, STIM003, STIM004, STIM005, STIM006, STIM007, STIM008 or STIM009, or comprises that LCDR2 with 1, 2, 3, 4 or 5 amino acid alterations, and/or
LCDR3 is the LCDR3 of STIM001, STIM002, STIM002-B, STIM003, STIM004, STIM005, STIM006, STIM007, STIM008 or STIM009 or comprises that LCDR3 with 1, 2, 3, 4 or 5 amino acid alterations.

81. The method of any one of clauses 77 to 80, wherein the antibody light chain CDRs are those of STIM001, STIM002, STIM002-B, STIM003, STIM004, STIM005, STIM006, STIM007, STIM008 or STIM009, or comprise the STIM001, STIM002, STIM002-B, STIM003, STIM004, STIM005, STIM006, STIM007, STIM008 or STIM009 light chain CDRs with 1, 2, 3, 4 or 5 amino acid alterations.

82. The method according to clause 81, wherein the antibody VL domain has the light chain CDRs of STIM003.

83. The method according to any of clauses 77 to 82, wherein the antibody comprises VH and/or VL domain framework regions of human germline gene segment sequences.

84. The method according to any of clauses 77 to 83, wherein the antibody comprises a VH domain which
(i) is derived from recombination of a human heavy chain V gene segment, a human heavy chain D gene segment and a human heavy chain J gene segment, wherein
   the V segment is IGHV1-18 (e.g., V1-18*01), IGVH3-20 (e.g. V3-20*d01), IGVH3-11 (e.g, V3-11*01) or IGVH2-5 (e.g., V2-5*10);
   the D gene segment is IGHD6-19 (e.g., IGHD6-19*01), IGHD3-10 (e.g., IGHD3-10*01) or IGHD3-9 (e.g., IGHD3-9*01); and/or
   the J gene segment is IGHJ6 (e.g., IGHJ6*02), IGHJ4 (e.g., IGHJ4*02) or IGHJ3 (e.g., IGHJ3*02), or
(ii) comprises framework regions FR1, FR2, FR3 and FR4, wherein
   FR1 aligns with human germline V gene segment IGHV1-18 (e.g., V1-18*01), IGVH3-20 (e.g. V3-20*d01), IGVH3-11 (e.g, V3-11*01) or IGVH2-5 (e.g., V2-5*10), optionally with 1, 2, 3, 4 or 5 amino acid alterations,
   FR2 aligns with human germline V gene segment IGHV1-18 (e.g., V1-18*01), IGVH3-20 (e.g. V3-20*d01), IGVH3-11 (e.g, V3-11*01) or IGVH2-5 (e.g., V2-5*10), optionally with 1, 2, 3, 4 or 5 amino acid alterations,
   FR3 aligns with human germline V gene segment IGHV1-18 (e.g., V1-18*01), IGVH3-20 (e.g. V3-20*d01), IGVH3-11 (e.g, V3-11*01) or IGVH2-5 (e.g., V2-5*10), optionally with 1, 2, 3, 4 or 5 amino acid alterations, and/or
   FR4 aligns with human germline J gene segment IGJH6 (e.g., JH6*02), IGJH4 (e.g., JH4*02) or IGJH3 (e.g., JH3*02), optionally with 1, 2, 3, 4 or 5 amino acid alterations.

85. The method according to any of clauses 77 to 84, wherein the antibody comprises an antibody VL domain which
(i) is derived from recombination of a human light chain V gene segment and a human light chain J gene segment, wherein
   the V segment is IGKV2-28 (e.g., IGKV2-28*01), IGKV3-20 (e.g., IGKV3-20*01), IGKV1D-39 (e.g., IGKV1D-39*01) or IGKV3-11 (e.g., IGKV3-11*01), and/or
   the J gene segment is IGKJ4 (e.g., IGKJ4*01), IGKJ2 (e.g., IGKJ2*04), IGLJ3 (e.g., IGKJ3*01) or IGKJ1 (e.g., IGKJ1*01); or
(ii) comprises framework regions FR1, FR2, FR3 and FR4, wherein
   FR1 aligns with human germline V gene segment IGKV2-28 (e.g., IGKV2-28*01), IGKV3-20 (e.g., IGKV3-20*01), IGKV1D-39 (e.g., IGKV1D-39*01) or IGKV3-11 (e.g., IGKV3-11*01), optionally with 1, 2, 3, 4 or 5 amino acid alterations,
   FR2 aligns with human germline V gene segment IGKV2-28 (e.g., IGKV2-28*01), IGKV3-20 (e.g., IGKV3-20*01), IGKV1D-39 (e.g., IGKV1D-39*01) or IGKV3-11 (e.g., IGKV3-11*01), optionally with 1, 2, 3, 4 or 5 amino acid alterations,
   FR3 aligns with human germline V gene segment IGKV2-28 (e.g., IGKV2-28*01), IGKV3-20 (e.g., IGKV3-20*01), IGKV1D-39 (e.g., IGKV1D-39*01) or IGKV3-11 (e.g., IGKV3-11*01), optionally with 1, 2, 3, 4 or 5 amino acid alterations, and/or
   FR4 aligns with human germline J gene segment IGKJ4 (e.g., IGKJ4*01), IGKJ2 (e.g., IGKJ2*04), IGKJ3 (e.g., IGKJ3*01) or IGKJ1 (e.g., IGKJ1*01), optionally with 1, 2, 3, 4 or 5 amino acid alterations.

86. The method according to any of clauses 77 to 85, wherein the antibody comprises an antibody VH domain which is the VH domain of STIM001, STIM002, STIM002-B, STIM003, STIM004, STIM005, STIM006, STIM007, STIM008 or STIM009, or which has an amino acid sequence at least 90% identical to the antibody VH domain sequence of STIM001, STIM002, STIM002-B, STIM003, STIM004, STIM005, STIM006, STIM007, STIM008 or STIM009.

87. The method according to any of clauses 77 to 86, wherein the antibody comprises an antibody VL domain which is the VL domain of STIM001, STIM002, STIM002-B, STIM003, STIM004, STIM005, STIM006, STIM007, STIM008 or STIM009, or which has an amino acid sequence at least 90% identical to the antibody VL domain sequence of STIM001, STIM002, STIM002-B, STIM003, STIM004, STIM005, STIM006, STIM007, STIM008 or STIM009.

88. The method according to clause 87, wherein the antibody comprises:
an antibody VH domain which is selected from the VH domain of STIM001, STIM002, STIM002-B, STIM003, STIM004, STIM005, STIM006, STIM007, STIM008 or STIM009, or which has an amino acid sequence at least 90% identical to the antibody VH domain sequence of STIM001, STIM002, STIM002-B, STIM003, STIM004, STIM005, STIM006, STIM007, STIM008 or STIM009, and
an antibody VL domain which is the VL domain of said selected antibody, or which has an amino acid sequence at least 90% identical to the antibody VL domain sequence of said selected antibody.

89. The method according to clause 88, wherein the antibody comprises the STIM003 VH domain and the STIM003 VL domain.

90. The method of any one of clauses 71 or 73 to 89, wherein antibody comprises an antibody constant region.

91. The method according to clause 90, wherein the constant region comprises a human heavy and/or light chain constant region.

92. The method according to clause 90 or clause 91, wherein the constant region is Fc effector positive.

93. The method according to clause 92, wherein the antibody comprises an Fc region that has enhanced ADCC, ADCP and/or CDC function compared with a native human Fc region.

94. The method according to any of clauses 90 to 93, wherein the antibody is an IgG1.

95. The method according to clause 91 or clause 92, wherein the antibody is afucosylated.

96. The method according to any of clauses 71 or 73 to 95 wherein the antibody is conjugated to a cytotoxic drug or pro-drug.

97. The method according to any of clauses 71 or 73 to 96, wherein the antibody is a multispecific antibody.

98. The method according to clause 71, wherein the anti-ICOS antibody is an antibody that binds the extracellular domain of human and mouse ICOS with an affinity (KD) of less than 50 nM as determined by surface plasmon resonance.

99. The method according to clause 98, wherein the antibody binds the extracellular domain of human and mouse ICOS with an affinity (KD) of less than 5 nM as determined by surface plasmon resonance.

100. The method according to clause 98 or clause 99, wherein the KD of binding the extracellular domain of human ICOS is within 10-fold of the KD of binding the extracellular domain of mouse ICOS.

101. The method of clause 71, wherein the anti-ICOS antibody is STIM0003.

102. The method of any preceding clause, wherein the PD-L1 inhibitor is an anti-PD-L1 antibody selected from the group consisting of atezoliumab (Roche), avelumab (Merck), durvalumab/Medi4736 (Medimmune), KN035, CK-301, AUNP12, CA-170, BMS-936559/MDX-1105 (BMS), FAZ-053 M7824, ABBV-368, LY-3300054, GNS-1480, YW243.55.S70, REGN3504 and any of the PD-L1 antibodies disclosed in WO2017/220990, WO2017/034916, WO2017/020291, WO2017/020858, WO2017/020801, WO2016/111645, WO2016/197367, WO2016/061142, WO2016/149201, WO2016/000619, WO2016/160792, WO2016/022630, WO2016/007235, WO2015/179654, WO2015/173267, WO2015/181342, WO2015/109124, WO2015/112805, WO2015/061668, WO2014/159562, WO2014/165082, WO2014/100079, WO2014/055897, WO2013/181634, WO2013/173223, WO2013/079174, WO2012/145493, WO2011/066389, WO2010/077634, WO2010/036959, WO2010/089411 or WO2007/005874.

103. The method of any preceding clause, wherein the PD-L1 inhibitor is an anti-PD-1 antibody selected from the group consisting of pembrolizumab, nivolumab, cemiplimab, JTX-401, spartalizumab (PDR001), camrelizumab (SHR1210), sintilimab (1B1308), tislelizumab (BGB-A317), toripalimab (JS 001), dostarlimab (TSR-042, WBP-285), INCMGA00012 (MGA012), AMP-224 and AMP-514, MEDI-0680/AMP514, PDR001, Lambrolizumab, BMS-936558, REGN2810, BGB-A317, BGB-108, PDR-001, SHR-1210, JS-001, JNJ-63723283, AGEN-2034, PF-06801591, genolimzumab, MGA-012 (INCMGA00012), IBI-308, BCD-100, TSR-042 ANA011, AUNP-12, KD033, MCLA-134, mDX400, muDX400, STI-A1110, AB011, 244C8, 388D4, XCE853, or pidilizumab/CT-011, or from any one of the anti-PD-1 antibodies described in WO2015/112800 & US2015/0203579 (including the antibodies in Tables 1 to 3), U.S. Pat. Nos. 9,394,365, 5,897,862 and 7,488,802, WO2017/087599 (including antibody SSI-361 and SHB-617), WO2017/079112, WO2017/071625 (including deposit C2015132, hybridoma LT004, and antibodies 6F5/6 F5 (Re), 6F5H1 L1 and 6F5 H2L2), WO2017/058859 (including PD1AB-1 to PD1AB-6), WO2017/058115 (including 67D9, c67D9, and hu67D9), WO2017/055547 (including 12819.15384, 12748.15381, 12748.16124, 12865.15377, 12892.15378, 12796.15376, 12777.15382, 12760.15375 and 13112.15380), WO2017/040790 (including AGEN2033w, AGEN2034w, AGEN2046w, AGEN2047w, AGEN2001w and AGEN2002w), WO2017/025051 & WO2017/024515 (including 1.7.3 hAb, 1.49.9 hAb, 1.103.11 hAb, 1.103.11-v2 hAb, 1.139.15 hAb and 1.153.7 hAb), WO2017/025016 & WO2017/024465 (including antibody A to antibody I), WO2017/020858 & WO2017/020291 (including 1.4.1, 1.14.4, 1.20.15 and 1.46.11), WO2017/019896 & WO2015/112900 & US2015/0210769 (including BAP049-hum01 to BAP049-hum16 and BAP049-Clone-A to BAP049-Clone-E), WO2017/019846 (including PD-1 mAb 1 to PD-1 mAb 15), WO2017/016497 (including MHC723, MHC724, MHC725, MHC728, MHC729, m136-M13, m136-M19, m245-M3, m245-M5 and m136-M14), WO2016/201051 (including antibody EH12.2H7, antibody hPD-1 mAb2, antibody hPD-1 mAb7, antibody hPD-1 mAb9, antibody hPD-1 mAb15, or an anti-PD-1 antibody selected from Table 1), WO2016/197497 (including DFPD1-1 to DFPD1-13), WO2016/197367 (including 2.74.15 and 2.74.15.hAb4 to 2.74.15.hAb8), WO2016/196173 (including the antibodies in Table 5, and FIGS. 1-5), WO2016/127179 (including R3A1, R3A2, R4B3, and R3D6), WO2016/077397 (including the antibodies described in Table 1 of Example 9), WO2016/106159 (including the murine antibodies in Table 3 of Example 2 and the humanised antibodies in Tables 7, 8 and 9 of Example 3), WO2016/092419 (including C1, C2, C3, EH12.1, mAb7-G4, mAb15-G4, mAb-AAA, mAb15-AAA), WO2016/068801 (including clone A3 and its variants and the other antibodies described in FIGS. 1 to 4), WO2016/014688 (including 10D1, 4C10, 7D3, 13F1, 15H5, 14A6, 22A5, 6E1, 5A8, 7A4, and 7A4D and the humanised antibodies of Examples 9/10), WO2016/015685 (including 10F8, BA08-1, BA-08-2 and 15H6), WO2015/091911 & WO2015/091910 (including the anti-canine PD-1 antibodies in Examples 2, 3 and 4), WO2015/091914 (including the anti-canine PD-1 antibodies in Table 3), WO2015/085847 (including mAb005, H005-1 to H005-4), WO2015/058573 (including cAB7), WO2015/036394 (including LOPD180), WO2015/035606 (including the antibodies in Table 1 of Example 2, in Tables 14, 15 and 16 of Example 7 and in tables 20, 21 and 22 of Example 11), WO2014/194302 (including GA2, RG1B3, RG1H10, RG2A7, RG2H10, SH-A4, RG4A6, GA1, GB1, GB6, GH1, A2, C7, H7, SH-A4, SH-A9, RG1H11, and RG6B), WO2014/179664 (including 9A2, 10B11, 6E9, APE1922, APE1923, APE1924, APE1950, APE1963 and APE2058), WO2014/206107 (including clone 1, 10, 11, 55, 64, 38, 39, 41 and 48), WO2012/135408 (including h409A11, h409A16, and h409A17), WO2012/145493 (including antibodies 1E3, 1E8, 1H3 and h1H3 Var 1 to h1H3 Var 14), WO2011/110621 (including antibody 949 and the modified versions disclosed in FIGS. 1 to 11), WO2011/110604 (including antibody 948 and the modified versions disclosed in FIGS. 3 to 11), WO2010/089411 (including CNCM deposit number 1-4122, 1-4080 or 1-4081), WO2010/036959 (including the antibodies in Table 1 of Example 1), WO2010/029435 & WO2010/029434 (including clones 2, 10 and 19), WO2008/156712 (including hPD-1.08A, hPD-1.09A, h409A11, h409A16 and h409A17 and the antibodies described in Example 2, Table H, Example 4 and table IV), WO2006/121168 (including clones 17D8, 4H1, 5C4, 4A11, 7D3, 5F4, and 2D3), WO2004/004771 and WO2004/056875 (including PD1-17, PD1-28, PD1-33, PD1-35, PD1-F2 and the Abs described in Table 1).

104. The method of any preceding clause, wherein the ICOS modulator is an IgG1 anti-ICOS antibody and/or the PD-L1 inhibitor is an IgG1 anti-PD-L1 antibody or an IgG1 anti-PD-1 antibody.

105. The method of clause 104, wherein the IgG1 anti-ICOS antibody and/or the IgG1 anti-PD-L1 antibody or anti-PD-1 antibody comprises a human IgG1 constant region comprising amino acid sequence SEQ ID NO: 340.

106. The method of any preceding clause, wherein the cancer is liver cancer, renal cell cancer, head and neck cancer, melanoma, non small cell lung cancer, diffuse large B-cell lymphoma, breast cancer, penile cancer, pancreatic cancer or oesophageal cancer

107. The method of clause 106, wherein the liver cancer is hepatocellular carcinoma.

108. The method of clause 106, wherein the head and neck cancer is metastatic squamous cell carcinoma.

109. The method of clause 106, wherein the breast cancer is triple negative breast cancer.

110. An ICOS modulator for use in the treatment of cancer in a patient, wherein the patient has a PD-L1 negative tumour or a tumour with low PD-L1 expression.

111. An ICOS modulator for use in the treatment of cancer in a patient, wherein the patient has previously received treatment for the cancer and the patient did not respond to the previous treatment or ceased responding to the previous treatment, wherein the previous treatment for the cancer was a PD-L1 inhibitor.

112. The ICOS modulator for use as claimed in clause 110 or clause 111, wherein the ICOS modulator is for use in combination with a PD-L1 inhibitor.

113. The ICOS modulator for use as claimed in any one of clauses 110 to 112, wherein the ICOS modulator is an agonistic anti-ICOS antibody.

114. The ICOS modulator for use as claimed in any one of clauses 110 to 113, wherein the ICOS modulator is a bispecific antibody that is an anti-ICOS agonist and an anti-PD-L1 antagonist or a bispecific antibody that is an anti-ICOS agonist and an anti-PD-1 antagonist.

115. The ICOS modulator for use according to any one clauses 110 to 114, wherein the method is the method of any one of clauses 1 to 109.

116. A combination of an ICOS modulator and a PD-L1 inhibitor for use in the treatment of cancer in a patient, wherein the patient has a PD-L1 negative tumour or a tumour with low PD-L1 expression.

117. A combination of an ICOS modulator and a PD-L1 inhibitor for use in the treatment of cancer in a patient, wherein the patient has previously received treatment for the cancer and the patient did not respond to the previous treatment or ceased responding to the previous treatment, wherein the previous treatment for the cancer was a PD-L1 inhibitor

118. The combination of clause 116 or clause 117, wherein the ICOS modulator is an agonistic anti-ICOS antibody.

119. The combination for use according to any one of clauses 116 to 118, wherein the method is the method of any one of clauses 1 to 109.

120. A modulator of ICOS and an inhibitor of PD-L1 for use in the treatment of cancer in a patient, wherein the patient has a PD-L1 negative cancer or a cancer with low PD-L1 expression.

121. A modulator of ICOS and an inhibitor of PD-L1 for use in the treatment of cancer in a patient, wherein the patient has previously received treatment for the cancer and the patient did not respond to the previous treatment or ceased responding to the previous treatment, wherein the previous treatment for the cancer was a PD-L1 inhibitor

122. The modulator of ICOS and inhibitor of PD-L1 for use according to any one of clauses 120 to 121, wherein the modulator of ICOS and inhibitor of PD-L1 is a bispecific antibody that specifically binds to ICOS and PD-1.

123. The modulator of ICOS and inhibitor of PD-L1 for use according to any one of clauses 120 to 122, wherein the ICOS modulator is an agonistic anti-ICOS antibody.

124. The modulator of ICOS and inhibitor of PD-L1 for use according to any one of clauses 120 to 123, wherein the method is the method of any one of clauses 1 to 109.

125. Use of an ICOS modulator in the manufacture of a medicament for the treatment of cancer in a patient, wherein the patient has a PD-L1 negative tumour or a tumour with low PD-L1 expression.

126. Use of an ICOS modulator in the manufacture of a medicament for the treatment of cancer in a patient, wherein the patient has previously received treatment for the cancer and the patient did not respond to the previous treatment or ceased responding to the previous treatment, wherein the previous treatment for the cancer was a PD-L1 inhibitor, and wherein the patient has a PD-L1 negative tumour or a tumour with low PD-L1 expression.

127. The use of clause 125 or clause 126, wherein the ICOS modulator is an agonistic anti-ICOS antibody.

128. The use of any one of clauses 125 to 127, wherein the ICOS modulator is a bispecific antibody that is an anti-ICOS agonist and an anti-PD-L1 antagonist or a bispecific antibody that is an anti-ICOS agonist and an anti-PD-1 antagonist.

129. The use of any one of clauses 125 to 127, wherein the treatment of cancer in a patient is the treatment by a method of any one of clauses 1 to 109.

130. Use of a combination of an ICOS modulator and a PD-L1 inhibitor in the manufacture of a medicament for the treatment of cancer in a patient, wherein the patient has a PD-L1 negative tumour or a tumour with low PD-L1 expression.

131. Use of a combination of an ICOS modulator and a PD-L1 inhibitor in the manufacture of a medicament for the treatment of cancer in a patient and the patient did not respond to the previous treatment or ceased responding to the previous treatment, wherein the patient has previously received treatment for the cancer, wherein the previous treatment for the cancer was a PD-L1 inhibitor, and wherein the patient has a PD-L1 negative tumour or a tumour with low PD-L1 expression.

132. Use of a modulator of ICOS and an inhibitor of PD-L1 in the manufacture of a medicament for the treatment of cancer in a patient, wherein the patient has a PD-L1 negative cancer or a cancer with low PD-L1 expression.

133. Use of a modulator of ICOS and an inhibitor of PD-L1 in the manufacture of a medicament for the treatment of cancer in a patient, wherein the patient has previously received treatment for the cancer, wherein the previous treatment for the cancer was a PD-L1 inhibitor, and wherein the patient has a PD-L1 negative tumour or a tumour with low PD-L1 expression.

134. The use of any one of clauses 132 to 133, wherein the tumour is HPV (Human papillomavirus) positive.

135. The use of any one of clauses 132 to 133, wherein the patient has, or has had, HPV (Human papillomavirus).

136. The use of any one of clauses 130 to 135, wherein the modulator of ICOS is an agonistic anti-ICOS antibody.

137. The use of any one of clauses 130 to 136, wherein the treatment of cancer in a patient is the treatment by a method of any one of clauses 1 to 109.

1. A method of treating cancer in a patient, wherein the patient has a PD-L1 negative tumour or a tumour with low PD-L1 expression, comprising administering to the patient a modulator of ICOS.

2-9. (canceled)

10. The method of claim 1, wherein the cancer is selected from the group consisting of head and neck squamous cell carcinoma, cervical cancer, anogenital cancer, melanoma, non small cell lung cancer, diffuse large B-cell lymphoma, breast cancer (e.g. triple negative breast cancer), penile cancer, pancreatic cancer, and oropharyngeal cancer.

11. The method of claim 1, wherein the tumour is HPV (Human papillomavirus) positive.

12-17. (canceled)

18. The method of claim 1, comprising administering a single dose of the ICOS modulator optionally followed by multiple doses of the PD-L1 inhibitor.

19-21. (canceled)

22. The method of claim 1, wherein the ICOS modulator is an anti-ICOS antibody comprising a VH domain comprises a set of heavy chain complementarity determining regions (HCDRs) HCDR1, HCDR2 and HCDR3, wherein
a. HCDR1 is the STIM003 HCDR1 having amino acid sequence SEQ ID NO: 405,
b. HCDR2 is the STIM003 HCDR2 having amino acid sequence SEQ ID NO: 406,
c. HCDR3 is the STIM003 HCDR3 having amino acid sequence SEQ ID NO: 407; and/or
wherein the VL domain comprises a set of light chain complementarity determining regions (LCDRs) LCDR1, LCDR2 and LCDR3, wherein:
d. LCDR1 is the STIM003 LCDR1 having amino acid sequence SEQ ID NO: 412,
e. LCDR2 is the STIM003 LCDR2 having amino acid sequence SEQ ID NO: 413,
f. LCDR3 is the STIM003 LCDR3 having amino acid sequence SEQ ID NO: 414.

23. The method of claim 1, wherein the ICOS modulator is an anti-ICOS antibody comprising a VH domain amino acid sequence is SEQ ID NO: 408 and/or wherein the VL domain amino acid sequence is SEQ ID NO: 415.

24-29. (canceled)

30. The method of claim 1, wherein antibody comprises an antibody constant region, optionally wherein the constant region comprises a human heavy and/or light chain constant region, optionally wherein the constant region is Fc effector positive.

31. The method of claim 1, wherein the antibody is a multispecific antibody.

32. (canceled)

33. The method of claim 1, wherein the PD-L1 inhibitor is an anti-PD-L1 antibody selected from the group consisting of atezoliumab (Roche), avelumab (Merck), durvalumab/Medi4736 (Medimmune), KN035, CK-301, AUNP12, CA-170, BMS-936559/MDX-1105 (BMS), FAZ-053 M7824, ABBV-368, LY-3300054, GNS-1480, YW243.55.570, REGN3504 and SP263.

34-36. (canceled)

37. A method of treating cancer in a patient comprising administering to the patient a modulator of ICOS, wherein:
a. the patient has a PD-L1 negative tumour or a tumour with low PD-L1 expression; or
b. the patient has previously received treatment for the cancer and the patient did not respond to the previous treatment or ceased responding to the previous treatment, wherein the previous treatment for the cancer was a PD-L1 inhibitor.

38. The ICOS modulator for use as claimed in claim 37, wherein the ICOS modulator is for use in combination with a PD-L1 inhibitor, optionally wherein the ICOS modulator is an agonistic anti-ICOS antibody, optionally wherein the ICOS modulator is a bispecific antibody that is an anti-ICOS agonist and an anti-PD-L1 antagonist or a bispecific antibody that is an anti-ICOS agonist and an anti-PD-1 antagonist.

39. (canceled)

40. The method of claim 37, wherein the ICOS modulator is an anti-ICOS antibody that binds the extracellular domain of human and/or mouse ICOS, comprising:
an antibody VH domain comprising complementarity determining regions (CDRs) HCDR1, HCDR2 and HCDR3, and
an antibody VL domain comprising complementarity determining regions LCDR1, LCDR2 and LCDR3, wherein:
HCDR1 is the HCDR1 of STIM001, STIM002, STIM002-B, STIM003, STIM004, STIM005, STIM006, STIM007, STIM008 or STIM009, or comprises that HCDR1 with 1, 2, 3, 4 or 5 amino acid alterations,
HCDR2 is the HCDR2 of STIM001, STIM002, STIM002-B, STIM003, STIM004, STIM005, STIM006, STIM007, STIM008 or STIM009, or comprises that HCDR2 with 1, 2, 3, 4 or 5 amino acid alterations, and/or
HCDR3 is the HCDR3 of STIM001, STIM002, STIM002-B, STIM003, STIM004, STIM005, STIM006, STIM007, STIM008 or STIM009 or comprises that HCDR3 with 1, 2, 3, 4 or 5 amino acid alterations; and/or
wherein LCDR1 is the LCDR1 of STIM001, STIM002, STIM002-B, STIM003, STIM004 STIM005, STIM006, STIM007, STIM008 or STIM009, or comprises that LCDR1 with 1, 2, 3, 4 or 5 amino acid alterations,
LCDR2 is the LCDR2 of STIM001, STIM002, STIM002-B, STIM003, STIM004, STIM005, STIM006, STIM007, STIM008 or STIM009, or comprises that LCDR2 with 1, 2, 3, 4 or 5 amino acid alterations, and/or
LCDR3 is the LCDR3 of STIM001, STIM002, STIM002-B, STIM003, STIM004, STIM005, STIM006, STIM007, STIM008 or STIM009 or comprises that LCDR3 with 1, 2, 3, 4 or 5 amino acid alterations.

41. The method of claim 37, wherein the antibody comprises an antibody VH domain which is the VH domain of STIM001, STIM002, STIM002-B, STIM003, STIM004, STIM005, STIM006, STIM007, STIM008 or STIM009, or which has an amino acid sequence at least 90% identical to the antibody VH domain sequence of STIM001, STIM002, STIM002-B, STIM003, STIM004, STIM005, STIM006, STIM007, STIM008 or STIM009; and/or wherein the antibody comprises an antibody VL domain which is the VL domain of STIM001, STIM002, STIM002-B, STIM003, STIM004, STIM005, STIM006, STIM007, STIM008 or STIM009, or which has an amino acid sequence at least 90% identical to the antibody VL domain sequence of STIM001, STIM002, STIM002-B, STIM003, STIM004, STIM005, STIM006, STIM007, STIM008 or STIM009.

42. A method of treating cancer in a patient who has previously received treatment for the cancer, wherein the previous treatment for the cancer was administration of a PD-L1 inhibitor and the patient did not respond to the previous treatment or ceased responding to the previous treatment, and wherein the patient has a PD-L1 negative tumour or a tumour with low PD-L1 expression, comprising administering to the patient a modulator of ICOS.

43. The method of claim 42, wherein the cancer is selected from the group consisting of head and neck squamous cell carcinoma, cervical cancer, anogenital cancer, melanoma, non small cell lung cancer, diffuse large B-cell lymphoma, breast cancer (e.g. triple negative breast cancer), penile cancer, pancreatic cancer, and oropharyngeal cancer.

44. The method of claim 42, wherein the VH domain comprises a set of heavy chain complementarity determining regions (HCDRs) HCDR1, HCDR2 and HCDR3, wherein
a. HCDR1 is the STIM003 HCDR1 having amino acid sequence SEQ ID NO: 405,
b. HCDR2 is the STIM003 HCDR2 having amino acid sequence SEQ ID NO: 406,
c. HCDR3 is the STIM003 HCDR3 having amino acid sequence SEQ ID NO: 407; and/or
wherein the VL domain comprises a set of light chain complementarity determining regions (LCDRs) LCDR1, LCDR2 and LCDR3, wherein:
d. LCDR1 is the STIM003 LCDR1 having amino acid sequence SEQ ID NO: 412,
e. LCDR2 is the STIM003 LCDR2 having amino acid sequence SEQ ID NO: 413,
f. LCDR3 is the STIM003 LCDR3 having amino acid sequence SEQ ID NO: 414.

45. The method of claim 42, wherein the VH domain amino acid sequence is SEQ ID NO: 408 and/or wherein the VL domain amino acid sequence is SEQ ID NO: 415.

46. The method of claim 42, wherein the cancer is selected from the group consisting of head and neck squamous cell carcinoma, cervical cancer, anogenital cancer, melanoma, non small cell lung cancer, diffuse large B-cell lymphoma, breast cancer (e.g. triple negative breast cancer), penile cancer, pancreatic cancer, and oropharyngeal cancer.

47. The method of claim 42, wherein the PD-L1 inhibitor is an anti-PD-L1 antibody selected from the group consisting of atezoliumab (Roche), avelumab (Merck), durvalumab/Medi4736 (Medimmune), KN035, CK-301, AUNP12, CA-170, BMS-936559/MDX-1105 (BMS), FAZ-053 M7824, ABBV-368, LY-3300054, GNS-1480, YW243.55.S70, REGN3504 and SP263.

48. The method of claim 42, wherein the antibody is a multispecific antibody.
